(19) United States
(12) Patent Application Publication
(10) Pub. No.: US 2015/0148311 A1
(43) Pub. Date: May 28, 2015

(54) NON-SYSTEMIC TGR5 AGONISTS

(71) Applicant: Ardelyx, Inc., Fremont, CA (US)

(72) Inventors: Jason G. Lewis, Castro Valley, CA (US); Nicholas Reich, Hillsboro, OR (US); Tao Chen, Palo Alto, CA (US); Jeffrey W. Jacobs, San Mateo, CA (US); Dominique Charmot, Campbell, CA (US); Marc Navre, Belmont, CA (US); Patricia Finn, Oakland, CA (US); Christopher Carreras, Belmont, CA (US); Andrew Spencer, Menlo Park, CA (US)

(21) Appl. No.: 14/310,989

(22) Filed: Jun. 20, 2014

Related U.S. Application Data
(63) Continuation of application No. PCT/US2012/071251, filed on Dec. 21, 2012.
(60) Provisional application No. 61/636,245, filed on Apr. 20, 2012.
(60) Provisional application No. 61/578,814, filed on Dec. 21, 2011.

(51) Int. Cl. C07D 241/42; C07D 403/06; C07D 417/06; A61K 45/06; C07D 413/06; A61K 31/5377; C07F 9/6547; A61K 31/675; A61K 31/498; A61K 31/541

(52) U.S. Cl. CPC C07D 241/42; A61K 31/498; C07D 403/06; C07D 417/06; A61K 31/541; C07D 413/06; A61K 31/5377; C07F 9/6547; A61K 31/675; A61K 45/06
USPC 514/80; 544/353; 514/249; 514/210.18; 544/62; 514/228.2; 544/116; 514/234.8; 544/337

(57) ABSTRACT

Compounds having the following structure (I):

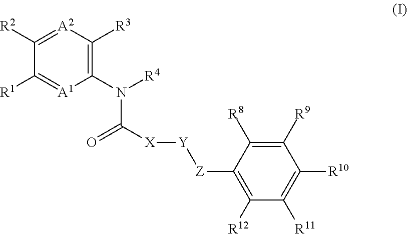

or a stereoisomer, tautomer, pharmaceutically acceptable salt or prodrug thereof, wherein R¹, R², R³, R⁴, R⁸, R⁹, R¹⁰, R¹¹, R¹², A¹, A², X, Y and Z are as defined herein. Uses of such compounds as TGR5 antagonists and for treatment of various indications, including Type II diabetes meletus are also provided.

### CROSS-REFERENCE TO RELATED APPLICATIONS

This application is a continuation of International PCT Application No. PCT/US2012/071251, filed Dec. 21, 2012, now pending, which claims the benefit under 35 U.S.C. §119(e) of U.S. Provisional Patent Application No. 61/578,814 filed Dec. 21, 2011 and U.S. Provisional Patent Application No. 61/636,245 filed Apr. 20, 2012. The foregoing applications are incorporated herein by reference in their entireties.

### BACKGROUND

1. Technical Field

The present invention is generally related to compounds having activity as TGR5 agonists, in particular TGR5 agonists which are not systemically available. The compounds are useful for treatment of any number of TGR5 mediated diseases or conditions, including diabetes.

2. Description of the Related Art

Diabetes mellitus is an ever-increasing threat to human health. For example, in the United States current estimates maintain that about 16 million people suffer from diabetes mellitus. Type II diabetes accounts for approximately 90-95% of diabetes cases, killing about 193,000 U.S. residents each year. Type II diabetes is the seventh leading cause of all deaths. In Western societies, Type II diabetes currently affects 6% of the adult population with world-wide frequency expected to grow by 6% per annum. Although there are certain inheritable traits that may predispose particular individuals to developing Type II diabetes, the driving force behind the current increase in incidence of the disease is the increased sedentary life-style, diet, and obesity now prevalent in developed countries. About 80% of diabetics with Type II diabetes are significantly overweight. Also, an increasing number of young people are developing the disease. Type II diabetes is now internationally recognized as one of the major threats to human health in the 21st century.

Type II diabetes manifests as inability to adequately regulate blood-glucose levels and may be characterized by a defect in insulin secretion or by insulin resistance. Namely, those who suffer from Type II diabetes have too little insulin or cannot use insulin effectively. Insulin resistance refers to the inability of the body tissues to respond properly to endogenous insulin. Insulin resistance develops because of multiple factors, including genetics, obesity, increasing age, and having high blood sugar over long periods of time. Type II diabetes can develop at any age, but most commonly becomes apparent during adulthood. However, the incidence of Type II diabetes in children is rising. In diabetics, glucose levels build up in the blood and urine causing excessive urination, thirst, hunger, and problems with fat and protein metabolism. If left untreated, diabetes mellitus may cause life-threatening complications, including blindness, kidney failure, and heart disease.

Type II diabetes is currently treated at several levels. A first level of therapy is through diet and/or exercise, either alone or in combination with therapeutic agents. Such agents may include insulin or pharmaceuticals that lower blood glucose levels. About 49% of individuals with Type II diabetes require oral medications, about 40% require insulin injections or a combination of insulin injections and oral medications, and 10% use diet and exercise alone.

Traditional therapies include: insulin secretagogues, such as sulphonylureas, which increase insulin production from pancreatic β-cells; glucose-lowering effectors, such as metformin which reduce glucose production from the liver; activators of the peroxisome proliferator-activated receptor γ (PPARγ), such as the thiazolidinediones, which enhance insulin action; and α-glucosidase inhibitors, which interfere with gut glucose production. There are, however, deficiencies associated with currently available treatments. For example sulphonylureas and insulin injections can be associated with hypoglycemic episodes and weight gain. Furthermore, patients often lose responsiveness to sulphonylureas over time. Metformin and α-glucosidase inhibitors often lead to gastrointestinal problems and PPARγ agonists tend to cause increased weight gain and edema.

More recently, new agents have been introduced to the market which prolong or mimic the effects of the naturally-secreted incretin hormones (Neumiller, JAm Pharm Assoc. 49(suppl 1):S16-S29, 2009). Incretins are a group of gastrointestinal hormones that are released from the beta cells of the pancreas when nutrients, especially glucose, are sensed in the gut. The two most important incretin hormones are glucose-dependent insulinotropic polypeptide (GIP) and GLP-1, which stimulate insulin secretion in a glucose-dependent manner and suppress glucagon secretion. However, GLP-1 itself is impractical as a clinical treatment for diabetes as it has a very short half-life in vivo. To address this, incretin-based agents currently available or in regulatory review for the treatment of T2DM are designed to achieve a prolonged incretin-action. For example, the dipeptidyl peptidase-4 inhibitors, such as sitagliptin, inhibit the normally rapid proteolytic breakdown of endogenous incretin hormones. There are also human-derived and synthetic incretin mimetics that are designed to be more stable and/or have a prolonged serum half-life compared to naturally secreted GLP-1, and include agents such as liraglutide and exenatide. In either approach, the goal is to provide a sustained incretin response and thus enhance glucose-dependent insulin secretion. It is the glucose-dependence of the insulin response that provides incretin therapies with low risk of hypoglycemia. In addition, GLP-1 can also delay gastric emptying and otherwise beneficially affect satiety and hence, weigh loss (Neumiller 2009).

Although significant progress has been made, there remains a need in the art for compounds which prolong or mimic the effects of the naturally-secreted incretin hormones such as GLP-1. The present invention fulfills this need and provides further related advantages.

### BRIEF SUMMARY

The present disclosure is directed to compounds having activity as TGR5 agonists and are useful for treatment of any number of TGR5 related diseases or conditions, for example metabolic diseases such as diabetes. The compounds are substantially active in the gastrointestinal (GI) tract to induce TGR5-mediated signaling, with such interaction causing an increase in the secretion of incretins, including GLP-1. In some embodiments, the compounds are designed to be substantially non-permeable or substantially non-bioavailable in the blood stream; that is, such compounds are designed to stimulate the TGR5-mediated release of GLP-1 into the bloodstream but be substantially non-systemic (e.g., systemic exposure levels below their TGR5 EC50) so as to limit their exposure to other internal organs (e.g., gall bladder, liver, heart, brain, etc.).

In accordance with one embodiment, there is provided a compound having the following structure (I):

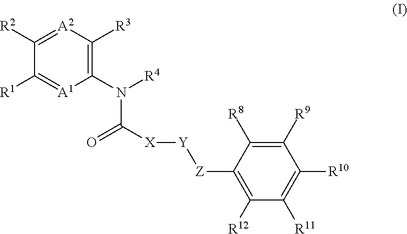

or a stereoisomer, tautomer, pharmaceutically acceptable salt or prodrug thereof, wherein R¹, R², R³, R⁴, R⁵, R⁶, R⁷, R⁸, R⁹, R¹⁰, R¹¹, R¹², A¹, A², X, Y and Z are as defined herein.

Pharmaceutical compositions comprising a compound of structure (I), a pharmaceutically acceptable carrier or adjuvant and optionally one or more additional therapeutically active agents are also provided.

The present disclosure is further directed to a method of treatment for increasing systemic levels of GLP-1, the method comprising administering a compound as disclosed herein, and/or a pharmaceutical composition as disclosed herein, to a mammal in need thereof. Such methods may be used, in particular, to treat various metabolic disorders, including for example diabetes (e.g., Type II diabetes mellitus). In other embodiments, the methods include treatment of gestational diabetes, impaired fasting glucose, impaired glucose tolerance, insulin resistance, hyperglycemia, obesity, metabolic syndrome and/or other diseases and/or conditions.

These and other aspects of the invention will be apparent upon reference to the following detailed description. To this end, various references are set forth herein which describe in more detail certain background information, procedures, compounds and/or compositions, and are each hereby incorporated by reference in their entirety.

### BRIEF DESCRIPTION OF THE DRAWINGS

FIG. 1 illustrates gallbladder emptying after oral administration of Examples 176 and 178.

FIG. 2 illustrates total (t)GLP-1 and (t)PYY levels in mouse plasma following oral dosing of Examples 176 and 178.

### DETAILED DESCRIPTION

### I. Definitions

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The terms below, as used herein, have the following meanings, unless indicated otherwise:

“Amino” refers to the —NH₂ radical.

“Aminocarbonyl” refers to the —C(═O)NH₂ radical.

“Carboxy” refers to the —CO₂H radical′

“Cyano” refers to the —CN radical.

“Hydroxy” or “hydroxyl” refers to the —OH radical.

“Imino” refers to the ═NH radical.

“Nitro” refers to the —NO₂ radical.

“Oxo” or “carbonyl” refers to the ═O radical.

“Thioxo” refers to the ═S radical.

“Guanidinyl” refers to the —NHC(═NH)NH₂ radical.

“Amidinyl” refers to the —C(═NH)NH₂ radical.

“Phosphate” refers to the —OP(═O)(OH)₂ radical.

“Phosphonate” refers to the —P(═O)(OH)₂ radical.

“Phosphinate” refers to the —PH(═O)OH radical.

“Sulfate” refers to the —OS(═O)₂OH radical.

“Sulfonate” or “hydroxysulfonyl” refers to the —S(═O)₂OH radical.

“Sulfinate” refers to the —S(═O)OH radical.

“Sulfonyl” refers to a moiety comprising a —SO₂— group. For example, “alkysulfonyl” or “alkylsulfone” refers to the —SO₂—Ra group, wherein Ra is an alkyl group as defined herein.

“Alkyl” refers to a straight or branched hydrocarbon chain radical consisting solely of carbon and hydrogen atoms, which is saturated or unsaturated (i.e., contains one or more double and/or triple bonds), having from one to seventy carbon atoms (C₁-C₇₀-alkyl), from one to twelve carbon atoms (C₁-C₁₂-alkyl) or one to seven carbon atoms (C₁-C₇-alkyl), and which is attached to the rest of the molecule by a single bond, e.g., methyl, ethyl, n-propyl, 1-methylethyl (iso-propyl), n-butyl, n-pentyl, 1,1-dimethylethyl (t-butyl), 3-methylhexyl, 2-methylhexyl, ethenyl, prop-1-enyl, but-1-enyl, pent-1-enyl, penta-1,4-dienyl, ethynyl, propynyl, butynyl, pentynyl, hexynyl, and the like. Unless stated otherwise specifically in the specification, an alkyl group may be optionally substituted, and an alkyl may optionally comprise one or more ether (—O—), thioether (—S—) or amine (—N<) bonds.

“Alkylene” or “alkylene chain” refers to a straight or branched divalent hydrocarbon chain linking the rest of the molecule to a radical group, consisting solely of carbon and hydrogen, which is saturated or unsaturated (i.e., contains one or more double and/or triple bonds), and having from one to seventy carbon atoms (C₁₋₇₀-alkylene), e.g., methylene, ethylene, propylene, n-butylene, ethenylene, propenylene, n-butenylene, propynylene, n-butynylene, and the like. The alkylene chain is attached to the rest of the molecule through a single or double bond and to the radical group through a single or double bond. The points of attachment of the alkylene chain to the rest of the molecule and to the radical group can be through one carbon or any two carbons within the chain. Unless stated otherwise specifically in the specification, an alkyl group may be optionally substituted, and an alkylene may optionally comprise one or more ether (—O—), thioether (—S—) or amine (—N<) bonds.

“Alkoxy” refers to a radical of the formula —ORₐ where Rₐ is an alkyl radical as defined above containing one to twelve carbon atoms. Unless stated otherwise specifically in the specification, an alkoxy group may be optionally substituted.

“Alkylamino” refers to a radical of the formula —NHRₐ or —NRₐRₐ where each Rₐ is, independently, an alkyl radical as defined above containing one to twelve carbon atoms. Unless stated otherwise specifically in the specification, an alkylamino group may be optionally substituted.

“Alkylaminocarbonyl” refers to the —C(═O)NHRₐ or —C(═O)NRₐRₐ radical, where each Rₐ is, independently, an alkyl radical as defined above containing one to twelve carbon atoms. Unless stated otherwise specifically in the specification, an alkylaminocarbonyl group may be optionally substituted.

“Alkoxyalkyl” refers to a radical of the formula —R_{b}ORₐ where Rₐ is an alkyl radical as defined and where R_{b} is an alkylene radical as defined. Unless stated otherwise specifically in the specification, an alkoxyalkyl group may be optionally substituted as described below.

“Alkylcarbonyl” refers to a radical of the formula —C(═O)Rₐ where Rₐ is an alkyl radical as defined above. Unless stated otherwise specifically in the specification, an alkylcarbonyl group may be optionally substituted as described below.

“Alkoxycarbonyl” refers to a radical of the formula —C(═O)ORₐ where Rₐ is an alkyl radical as defined. Unless stated otherwise specifically in the specification, an alkyloxycarbonyl group may be optionally substituted as described below.

“Alkylcarbonyloxy” refers to a radical of the formula —OC(═O)Rₐ where Rₐ is an alkyl radical as defined above. Unless stated otherwise specifically in the specification, an alkyloxycarbonyl group may be optionally substituted as described below.

“Carboxylalkyl” refers to a radical of the formula —RₐCO₂H where Rₐ is an alkyl radical as defined above. Unless stated otherwise specifically in the specification, a carboxyalkyl group may be optionally substituted as described below.

“Thioalkyl” refers to a radical of the formula —SRₐ where Rₐ is an alkyl radical as defined above containing one to twelve carbon atoms. Unless stated otherwise specifically in the specification, a thioalkyl group may be optionally substituted.

“Aryl” refers to a hydrocarbon ring system radical comprising hydrogen, 6 to 18 carbon atoms and at least one aromatic ring. For purposes of this invention, the aryl radical may be a monocyclic, bicyclic, tricyclic or tetracyclic ring system, which may include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” or the prefix “ar-” (such as in “aralkyl”) is meant to include aryl radicals that are optionally substituted.

“Aralkyl” refers to a radical of the formula —R_{b}—R_{c} where R_{b} is an alkylene chain as defined above and R_{c} is one or more aryl radicals as defined above, for example, benzyl, diphenylmethyl and the like. Unless stated otherwise specifically in the specification, an aralkyl group may be optionally substituted.

“Cycloalkyl” or “carbocyclic ring” refers to a stable non-aromatic monocyclic or polycyclic hydrocarbon radical consisting solely of carbon and hydrogen atoms, which may include fused or bridged ring systems, having from three to fifteen carbon atoms, preferably having from three to ten carbon atoms, and which is saturated or unsaturated and attached to the rest of the molecule by a single bond. A “C₃₋₇-cycloalkyl refers to a cycloalkyl having from 3 to 7 carbon atoms in the cycloalkyl ring. Monocyclic radicals include, for example, cyclopropyl, cyclobutyl, cyclopentyl, cyclohexyl, cycloheptyl, and cyclooctyl. Polycyclic radicals include, for example, adamantyl, norbornyl, decalinyl, 7,7-dimethyl-bicyclo[2.2.1]heptanyl, and the like. Unless otherwise stated specifically in the specification, a cycloalkyl group may be optionally substituted.

“Cycloalkylalkyl” refers to a radical of the formula —R_{b}R_{d} where R_{b} is an alkylene chain as defined above and R_{d} is a cycloalkyl radical as defined above. Unless stated otherwise specifically in the specification, a cycloalkylalkyl group may be optionally substituted.

“Fused” refers to any ring structure described herein which is fused to an existing ring structure in the compounds of the invention. When the fused ring is a heterocyclyl ring or a heteroaryl ring, any carbon atom on the existing ring structure which becomes part of the fused heterocyclyl ring or the fused heteroaryl ring may be replaced with a nitrogen atom.

“Halo” or “halogen” refers to bromo, chloro, fluoro or iodo.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. A “C₃₋₇-haloalkyl refers to a haloalkyl having from 3 to 7 carbon atoms. Unless stated otherwise specifically in the specification, a haloalkyl group may be optionally substituted.

“Heterocyclyl” or “heterocyclic ring” or “heterocycle” refers to a stable 3- to 18-membered non-aromatic ring radical which consists of two to twelve carbon atoms and from one to six heteroatoms selected from the group consisting of nitrogen, oxygen and sulfur. Unless stated otherwise specifically in the specification, the heterocyclyl radical may be a monocyclic, bicyclic, tricyclic or tetracyclic ring system, which may include fused or bridged ring systems; and the nitrogen, carbon or sulfur atoms in the heterocyclyl radical may be optionally oxidized; the nitrogen atom may be optionally quaternized; and the heterocyclyl radical may be partially or fully saturated.

Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group may be optionally substituted.

“N-heterocyclyl” refers to a heterocyclyl radical as defined above containing at least one nitrogen and where the point of attachment of the heterocyclyl radical to the rest of the molecule is through a nitrogen atom in the heterocyclyl radical. Unless stated otherwise specifically in the specification, a N-heterocyclyl group may be optionally substituted.

“Heterocyclylalkyl” refers to a radical of the formula —R_{b}Rₑ, where R_{b} is an alkylene chain as defined above and Rₑ is a heterocyclyl radical as defined above, and if the heterocyclyl is a nitrogen-containing heterocyclyl, the heterocyclyl may be attached to the alkyl radical at the nitrogen atom. Unless stated otherwise specifically in the specification, a heterocyclylalkyl group may be optionally substituted.

“Heteroaryl” refers to a 5- to 14-membered ring system radical comprising hydrogen atoms, one to thirteen carbon atoms, one to six heteroatoms selected from the group consisting of nitrogen, oxygen and sulfur, and at least one aromatic ring. For purposes of this invention, the heteroaryl radical may be a monocyclic, bicyclic, tricyclic or tetracyclic ring system, which may include fused or bridged ring systems; and the nitrogen, carbon or sulfur atoms in the heteroaryl radical may be optionally oxidized; the nitrogen atom may be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in the specification, a heteroaryl group may be optionally substituted.

“N-heteroaryl” refers to a heteroaryl radical as defined above containing at least one nitrogen and where the point of attachment of the heteroaryl radical to the rest of the molecule is through a nitrogen atom in the heteroaryl radical. Unless stated otherwise specifically in the specification, an N-heteroaryl group may be optionally substituted.

“Heteroarylalkyl” refers to a radical of the formula —R_{b}R_{f} where R_{b} is an alkylene chain as defined above and R_{f} is a heteroaryl radical as defined above. Unless stated otherwise specifically in the specification, a heteroarylalkyl group may be optionally substituted.

The term “substituted” used herein means any of the above groups (e.g., alkyl, alkylene, alkoxy, alkylamino, alkylaminocarbonyl, alkoxyalkyl, alkylcarbonyl, alkoxycarbonyl, alkylcarbonyloxy, carboxylalkyl, thioalkyl, aryl, aralkyl, cycloalkyl, cycloalkylalkyl, haloalkyl, heterocyclyl, N-heterocyclyl, heterocyclylalkyl, heteroaryl, N-heteroaryl and/or heteroarylalkyl) wherein at least one hydrogen atom is replaced by a bond to a non-hydrogen atoms such as, but not limited to: a halogen atom such as F, Cl, Br, and I; an oxygen atom in groups such as hydroxyl groups, carboxyl groups, guanidine groups, imidine groups, phosphate groups, phosphinate groups, phosphonate groups, sulfate groups, sulfinate groups, alkoxy groups, ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkoxy, alkylamino, thioalkyl, aryl, aralkyl, cycloalkyl, cycloalkylalkyl, haloalkyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkoxy, alkylamino, thioalkyl, aryl, aralkyl, cycloalkyl, cycloalkylalkyl, haloalkyl, heterocyclyl, N-heterocyclyl, heterocyclylalkyl, heteroaryl, N-heteroaryl and/or heteroarylalkyl group. In addition, each of the foregoing substituents may also be optionally substituted with one or more of the above substituents.

Prodrugs of compounds of structure (I) are included in the scope of the invention. “Prodrug” is meant to indicate a compound that may be converted under physiological conditions or by solvolysis to a biologically active compound of the invention. Thus, the term “prodrug” refers to a metabolic precursor of a compound of the invention that is pharmaceutically acceptable. A prodrug may be inactive when administered to a subject in need thereof, but is converted in vivo to an active compound of the invention. Prodrugs are typically rapidly transformed in vivo to yield the parent compound of the invention, for example, by hydrolysis in blood. The prodrug compound often offers advantages of solubility, tissue compatibility or delayed release in a mammalian organism (see, Bundgaard, H., Design of Prodrugs (1985), pp. 7-9, 21-24 (Elsevier, Amsterdam)). A discussion of prodrugs is provided in Higuchi, T., et al., Pro-drugs as Novel Drug Delivery Systems, A.C.S. Symposium Series, Vol. 14, 1975, and in Bioreversible Carriers in Drug Design, Ed. Edward B. Roche, American Pharmaceutical Association and Pergamon Press, 1987.

The term “prodrug” is also meant to include any covalently bonded carriers, which release the active compound of the invention in vivo when such prodrug is administered to a mammalian subject. Prodrugs of a compound of the invention may be prepared by modifying functional groups present in the compound of the invention in such a way that the modifications are cleaved, either in routine manipulation or in vivo, to the parent compound of the invention. Prodrugs include compounds of the invention wherein a hydroxy, amino or mercapto group is bonded to any group that, when the prodrug of the compound of the invention is administered to a mammalian subject, cleaves to form a free hydroxy, free amino or free mercapto group, respectively. Examples of prodrugs include, but are not limited to, acetate, formate and benzoate derivatives of alcohol or amide derivatives of amine functional groups in the compounds of the invention and the like.

The invention disclosed herein is also meant to encompass all pharmaceutically acceptable compounds of structure (I) being isotopically-labeled by having one or more atoms replaced by an atom having a different atomic mass or mass number. Examples of isotopes that can be incorporated into the disclosed compounds include isotopes of hydrogen, carbon, nitrogen, oxygen, phosphorous, sulfur, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²⁵I, and ¹²⁵I, respectively. These radiolabelled compounds could be useful to help determine or measure the effectiveness of the compounds, by characterizing, for example, the site or mode of action, or binding affinity to pharmacologically important site of action. Certain isotopically-labelled compounds of structure (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds of structure (I) can generally be prepared by conventional techniques known to those skilled in the art or by processes analogous to those described in the Preparations and Examples as set out below using an appropriate isotopically-labeled reagent in place of the non-labeled reagent previously employed.

The invention disclosed herein is also meant to encompass the in vivo metabolic products of the disclosed compounds. Such products may result from, for example, the oxidation, reduction, hydrolysis, amidation, esterification, and the like of the administered compound, primarily due to enzymatic processes. Accordingly, the invention includes compounds produced by a process comprising administering a compound of this invention to a mammal for a period of time sufficient to yield a metabolic product thereof. Such products are typically identified by administering a radiolabelled compound of the invention in a detectable dose to an animal, such as rat, mouse, guinea pig, monkey, or to human, allowing sufficient time for metabolism to occur, and isolating its conversion products from the urine, blood or other biological samples.

“Stable compound” and “stable structure” are meant to indicate a compound that is sufficiently robust to survive isolation to a useful degree of purity from a reaction mixture, and formulation into an efficacious therapeutic agent.

“Mammal” includes humans and both domestic animals such as laboratory animals and household pets (e.g., cats, dogs, swine, cattle, sheep, goats, horses, rabbits), and non-domestic animals such as wildlife and the like.

“Optional” or “optionally” means that the subsequently described event of circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

The present invention includes pharmaceutically acceptable salts of compounds of structure (I). “Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

Often crystallizations produce a solvate of the compound of the invention. As used herein, the term “solvate” refers to an aggregate that comprises one or more molecules of a compound of the invention with one or more molecules of solvent. The solvent may be water, in which case the solvate may be a hydrate. Alternatively, the solvent may be an organic solvent. Thus, the compounds of the present invention may exist as a hydrate, including a monohydrate, dihydrate, hemihydrate, sesquihydrate, trihydrate, tetrahydrate and the like, as well as the corresponding solvated forms. The compound of the invention may be true solvates, while in other cases, the compound of the invention may merely retain adventitious water or be a mixture of water plus some adventitious solvent.

A “co-crystal” of a compound of the invention can also be formed. Co-crystallization can alter the molecular interactions and composition of pharmaceutical materials, and provide unique drug properties. Co-crystals consist of a compound of the invention and a typically stoichiometric amount of a pharmaceutically acceptable co-crystal former. Pharmaceutical co-crystals are nonionic supramolecular complexes and can be used to address physical property issues such as solubility, stability and bioavailability in pharmaceutical development without changing the chemical composition of the compound of the invention.

A “pharmaceutical composition” refers to a formulation of a compound of the invention and a medium generally accepted in the art for the delivery of the biologically active compound to mammals, e.g., humans. Such a medium includes all pharmaceutically acceptable carriers, diluents or excipients therefor.

“Effective amount” or “therapeutically effective amount” refers to that amount of a compound of the invention which, when administered to a mammal, preferably a human, is sufficient to effect treatment, as defined below, of agonizing TGR5 in the mammal, preferably a human. The amount of a compound of the invention which constitutes a “therapeutically effective amount” will vary depending on the compound, the condition and its severity, the manner of administration, and the age of the mammal to be treated, but can be determined routinely by one of ordinary skill in the art having regard to his own knowledge and to this disclosure.

“Treating” or “treatment” as used herein covers the treatment of the disease or condition of interest in a mammal, preferably a human, having the disease or condition of interest, and includes:

(i) preventing the disease or condition from occurring in a mammal, in particular, when such mammal is predisposed to the condition but has not yet been diagnosed as having it;

(ii) inhibiting the disease or condition, i.e., arresting its development;

(iii) relieving the disease or condition, i.e., causing regression of the disease or condition; or

(iv) relieving the symptoms resulting from the disease or condition, i.e., relieving pain without addressing the underlying disease or condition. As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

The compounds of the invention, or their pharmaceutically acceptable salts may contain one or more asymmetric centers and may thus give rise to enantiomers, diastereomers, and other stereoisomeric forms that may be defined, in terms of absolute stereochemistry, as (R)— or (S)— or, as (D)- or (L)- for amino acids. The present invention is meant to include all such possible isomers, as well as their racemic and optically pure forms. Optically active (+) and (−), (R)— and (S)—, or (D)- and (L)- isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centres of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present invention contemplates various stereoisomers and mixtures thereof and includes “enantiomers”, which refers to two stereoisomers whose molecules are nonsuperimposeable mirror images of one another.

A “tautomer” refers to a proton shift from one atom of a molecule to another atom of the same molecule. The present invention includes tautomers of any said compounds.

The chemical naming protocol and structure diagrams used herein are a modified form of the I.U.P.A.C. nomenclature system, using the “IUPAC Naming Plugin” software program (ChemAxon) and/or ChemDraw software Struct=Name Pro 11.0 program (CambridgeSoft). For complex chemical names employed herein, a substituent group is named before the group to which it attaches. For example, cyclopropylethyl comprises an ethyl backbone with cyclopropyl substituent.

### II. Compounds

As noted above, in one embodiment of the present invention, compounds having activity as TGR5 agonists are provided, the compounds having the following structure (I):

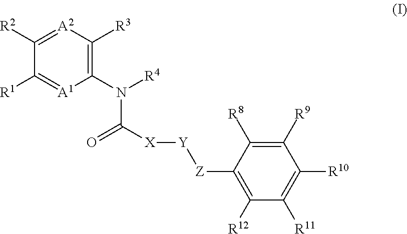

or a stereoisomer, tautomer, pharmaceutically acceptable salt or prodrug thereof, wherein:
X is CR⁵⁰R⁵¹ wherein:
   R⁵⁰ and R⁵¹ are the same or different and independently selected from H and C₁₋₇-alkyl, or
   R⁵⁰ and R⁵¹ taken together with the C atom to which they are attached form a cycloalkyl or heterocyclyl, wherein the cycloalkyl or heterocyclyl are optionally substituted by one or two groups selected from halogen, hydroxy, oxo, C₁₋₇-alkyl, C₁₋₇-haloalkyl, C₁₋₇-alkylcarbonyl, C₁₋₇-alkyloxycarbonyl, C₁₋₇-alkoxy, C₁₋₇-alkoxyalkyl, (R^{a})₂(R^{b})N— and C₁₋₇-alkyl-S(O)₀₋₂—, wherein each R^{a} is independently, at each occurrence, hydrogen or C₁₋₇-alkyl and R^{b} is an electron pair, hydrogen or C₁₋₇-alkyl;
Y is CR⁶⁰R⁶¹, O, NR⁶² or a direct bond, provided that when Y is O, Z is not 0 or S(O)₀₋₂, wherein:
   R⁶⁰ and R⁶¹ are the same or different and independently selected from H and C₁₋₇-alkyl; and
   R⁶² is selected from H, C₁₋₇-alkyl, C₁₋₇-alkylcarbonyl, aminocarbonyl, C₁₋₇-alkylaminocarbonyl, C₁₋₇-alkylsulfone, cycloalkylalkyl, cycloalkyl, aralkyl and aryl, wherein the C₁₋₇-alkyl, C₁₋₇-alkylcarbonyl, aminocarbonyl, C₁₋₇-alkylaminocarbonyl, C₁₋₇-alkylsulfone, cycloalkylalkyl, cycloalkyl, aralkyl and aryl are optionally substituted with one or more substitutents selected from halogen, hydroxy, oxo, C₁₋₇-alkyl, C₁₋₇-haloalkyl, C₁₋₇-alkylcarbonyl, C₁₋₇-alkyloxycarbonyl, C₁₋₇-alkoxy, C₁₋₇-alkoxyalkyl, (Ra)₂(R^{b})N— and C₁₋₇-alkyl-S(O)₀₋₂—, wherein each R^{a} is independently, at each occurrence, hydrogen or C₁₋₇-alkyl and R^{b} is an electron pair, hydrogen or C₁₋₇-alkyl;
or X and Y taken together form a cycloalkyl, heterocyclyl, aryl or heteroaryl, wherein the cycloalkyl, heterocyclyl, aryl or heteroaryl are optionally substituted by one or two groups selected from halogen, hydroxy, oxo, C₁₋₇-alkyl, C₁₋₇-alkylcarbonyl, C₁₋₇-alkyloxycarbonyl, C₁₋₇-alkoxy, C₁₋₇-alkoxyalkyl, (Ra)₂(R^{b})N— and C₁₋₇-alkyl-S(O)₀₋₂—, wherein R^{a} is independently, at each occurrence, hydrogen or C₁₋₇-alkyl and R^{b} is an electron pair, hydrogen or C₁₋₇-alkyl, and provided that when X and Y form phenyl, pyridyl, pyridyl-N-oxide or pyrimidinyl then Z is not 0;
Z is CR⁷⁰R⁷¹, 0, S(O)₀₋₂ or a direct bond, wherein:
   R⁷⁰ and R⁷¹ are the same or different and independently selected from H or C₁₋₇-alkyl;
   or R⁷⁰ and R⁷¹ taken together to form oxo (═O);
   or Z and R⁸ or R¹² taken together form a cycloalkyl or heterocyclyl, wherein the cycloalkyl or heterocyclyl are optionally substituted by one or two groups selected from halogen, hydroxy, oxo, C₁₋₇-alkyl, C₁₋₇-haloalkyl, C₁₋₇-alkylcarbonyl, C₁₋₇-alkyloxycarbonyl, C₁₋₇-alkoxy, C₁₋₇-alkoxyalkyl, (Ra)₂(R^{b})N— and C₁₋₇-alkyl-S(O)₀₋₂—, wherein each R^{a} is independently, at each occurrence, hydrogen or C₁₋₇-alkyl and R^{b} is an electron pair, hydrogen or C₁₋₇-alkyl;
A¹ is CR¹³ or N;
A² is CR¹⁴ or N, wherein:
   R¹³ and R¹⁴ are the same or different and independently selected from: hydrogen, C₁₋₇-alkyl, halogen, C₁₋₇-haloalkyl, cyano, C₁₋₇-alkoxy, amino and —S(O)₀₋₂—C₁₋₇-alkyl;
R¹ and R² are the same or different and independently selected from: hydrogen, C₁₋₇-alkyl, halogen, halogen-C₁₋₇-alkyl, cyano and C₁₋₇-alkoxy;
R³ is selected from: hydrogen, C₁₋₇-alkyl, halogen, C₁₋₇-haloalkyl, C₁₋₇-alkoxy, cyano, C₃₋₇-cycloalkyl, —O—C₃₋₇-cycloalkyl, —O—C₁₋₇-alkyl-C₃₋₇-cycloalkyl, —S(O)₀₋₂—C₁₋₇-alkyl, N-heterocyclyl, five-membered heteroaryl, phenyl and —NR¹⁵R¹⁶, wherein R¹⁵ and R¹⁶ are the same or different and independently selected from hydrogen, C₁₋₇-alkyl and C₃₋₇-cycloalkyl;
R⁴ is selected from: hydrogen, C₁₋₇-alkyl, halogen-C₁₋₇-alkyl, and C₃₋₇-cycloalkyl;
or R³ and R⁴ or R³ and R¹⁴ together are -L¹-(CR¹⁷R¹⁸)ₙ— and form part of a ring, wherein:
   L¹ is selected from: —CR¹⁹R²⁰—, O, S(O)₀₋₂, C═O and NR²¹;
   R¹⁷ and R¹⁸ are the same or different and independently selected from hydrogen and C₁₋₇-alkyl;
   or R¹⁷ and R¹⁸ together with the C atom to which they are attached form an oxo moiety;
   or R¹⁷ or R¹⁸ together with an adjacent R¹⁷, R¹⁸, R¹⁹ or R²⁰ and the C atoms to which they are attached form C═C;
   R¹⁹ and R²⁰ are the same or different and independently selected from: hydrogen, hydroxyl, N(R²¹)₂, C₁₋₇-alkyl, C₁₋₇-alkoxycarbonyl, unsubstituted heterocyclyl, and heterocyclyl substituted by one or two groups selected from halogen, hydroxy and C₁₋₇-alkyl,
   or R¹⁹ and R²⁰ together with the C atom to which they are attached form a cyclopropyl or oxetanyl ring or together form a ═CH₂ or ═CF₂ group; and
   R²¹ is independently, at each occurrence, selected from the group consisting of: hydrogen, C₁₋₇-alkyl, halogen-C₁₋₇-alkyl, C₃₋₇-cycloalkyl and C₃₋₇-cycloalkyl-C₁₋₇-alkyl, wherein C₃₋₇-cycloalkyl is unsubstituted or substituted by carboxyl-C₁₋₇-alkyl or C₁₋₇-alkoxycarbonyl, heterocyclyl, heterocyclyl-C₁₋₇-alkyl, heteroaryl, heteroaryl-C₁₋₇-alkyl, carboxyl-C₁₋₇-alkyl, C₁₋₇-alkoxycarbonyl-C₁₋₇-alkyl, C₁₋₇-alkylcarbonyloxy-C₁₋₇-alkyl, C₁₋₇-alkylsulfonyl, phenyl, wherein phenyl is unsubstituted or substituted by carboxyl-C₁₋₇-alkyl or C₁₋₇-alkoxycarbonyl, phenylcarbonyl, wherein phenyl is unsubstituted or substituted by carboxyl-C₁₋₇-alkyl or C₁₋₇-alkoxycarbonyl, and phenylsulfonyl, wherein phenyl is unsubstituted or substituted by carboxyl-C₁₋₇-alkyl or C₁₋₇-alkoxycarbonyl;
   or R²¹ and a R¹⁷ together are —(CH₂)₃— and form part of a ring;
   or R²¹ together with a pair of R¹² and R¹⁸ are —CH═CH—CH═ and form part of a ring; and
   n is 1, 2 or 3;
R⁸, R⁹, R¹⁰, R¹¹ and R¹² are the same or different and independently selected from: Q, hydrogen, C₁₋₇-alkyl, C₂₋₇-alkenyl, C₂₋₇-alkynyl, halogen, halogen-C₁₋₇-alkyl, C₁₋₇-alkoxy, halogen-C₁₋₇-alkoxy, hydroxy, hydroxy-C₁₋₇-alkoxy, hydroxy-C₁₋₇-alkyl, hydroxy-C₃₋₇-alkenyl, hydroxy-C₃₋₇-alkynyl, cyano, carboxyl, C₁₋₇-alkoxycarbonyl, amino carbonyl, carboxyl-C₁₋₇-alkyl, carboxyl-C₂₋₇-alkenyl, carboxyl-C₂₋₇-alkynyl, C₁₋₇-alkoxycarbonyl-C₁₋₇-alkyl, C₁₋₇-alkoxycarbonyl-C₂₋₇-alkenyl, C₁₋₇-alkoxycarbonyl-C₂₋₇-alkynyl, carboxyl-C₁₋₇-alkoxy, C₁₋₇-alkoxycarbonyl-C₁₋₇-alkoxy, carboxyl-C₁₋₇-alkyl-aminocarbonyl, carboxyl-C₁₋₇-alkyl-(C₁₋₇-alkylamino)-carbonyl, C₁₋₇-alkoxycarbonyl-C₁₋₇-alkyl-aminocarbonyl, C₁₋₇-alkoxycarbonyl-C₁₋₇-alkyl-(C₁₋₇-alkylamino)-carbonyl, carboxyl-C₁₋₇-alkyl-aminocarbonyl-C₁₋₇-alkyl, carboxyl-C₁₋₇-alkyl-(C₁₋₇-alkylamino)-carbonyl-C₁₋₇-alkyl, C₁₋₇-alkoxycarbonyl-C₁₋₇-alkyl-aminocarbonyl-C₁₋₇-alkyl, C₁₋₇-alkoxycarbonyl-C₁₋₇-alkyl-(C₁₋₇-alkylamino)-carbonyl-C₁₋₇-alkyl, hydroxy-C₁₋₇-alkyl-aminocarbonyl, di-(hydroxy-C₁₋₇-alkyl)aminocarbonyl, aminocarbonyl-C₁₋₇-alkyl-amino carbonyl, hydroxysulfonyl-C₁₋₇-alkyl-aminocarbonyl, hydroxysulfonyl-C₁₋₇-alkyl-(C₁₋₇-alkyl-amino)-carbonyl, di-(C₁₋₇-alkoxycarbonyl-C₁₋₇-alkyl)-methylaminocarbonyl, phenyl, wherein phenyl is unsubstituted or substituted by one to three groups selected from halogen, C₁₋₇-alkoxy, carboxyl or C₁₋₇-alkoxycarbonyl, phenyl-carbonyl, wherein phenyl is unsubstituted or substituted by one to three groups selected from halogen, C₁₋₇-alkoxy, carboxyl or C₁₋₇-alkoxycarbonyl, phenyl-aminocarbonyl, wherein phenyl is unsubstituted or substituted by one to three groups selected from halogen, C₁₋₇-alkoxy, carboxyl or C₁₋₇-alkoxycarbonyl, phenyl-C₁₋₇-alkyl, wherein phenyl is unsubstituted or substituted by one to three groups selected from halogen C₁₋₇-alkoxy, carboxyl or C₁₋₇-alkoxycarbonyl, phenyl-C₂₋₇-alkynyl, wherein phenyl is unsubstituted or substituted by one to three groups selected from halogen, C₁₋₇-alkoxy, carboxyl or C₁₋₇-alkoxycarbonyl, heteroaryl, wherein heteroaryl is unsubstituted or substituted by one to three groups selected from halogen C₁₋₇-alkoxy, carboxyl or C₁₋₇-alkoxycarbonyl, heteroaryl-carbonyl, wherein heteroaryl is unsubstituted or substituted by one to three groups selected from halogen, C₁₋₇-alkoxy, carboxyl or C₁₋₇-alkoxycarbonyl, heteroaryl-aminocarbonyl, wherein heteroaryl is unsubstituted or substituted by one to three groups selected from halogen, C₁₋₇-alkoxy, carboxyl or C₁₋₇-alkoxycarbonyl, heteroaryl-C₁₋₇-alkyl, wherein heteroaryl is unsubstituted or substituted by one to three groups selected from halogen, C₁₋₇-alkyl, C₁₋₇-alkoxy, carboxyl or C₁₋₇-alkoxycarbonyl, heteroaryl-C₁₋₇-alkyl-aminocarbonyl, wherein heteroaryl is unsubstituted or substituted by one to three groups selected from halogen, C₁₋₇-alkoxy, carboxyl or C₁₋₇-alkoxycarbonyl, heteroaryl-carbonyl-C₁₋₇-alkyl, wherein heteroaryl is unsubstituted or substituted by one to three groups selected from halogen, C₁₋₇-alkoxy, carboxyl or C₁₋₇-alkoxycarbonyl, and cycloalkyl, wherein cycloalkyl is unsubstituted or substituted by one to three groups selected from halogen, C₁₋₇-alkoxy, carboxyl or C₁₋₇-alkoxycarbonyl;
Q is:

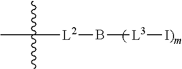

wherein:
   L² and each L³ are either the same or different and independently absent, —O—, —NR⁸⁰—, —S—, —NR⁸⁰C(═O)—, —C(═O)NR⁸⁰—, —NR⁸⁰C(═O)NR⁸⁰—, —SO₂NR⁸⁰—, —NR⁸⁰SO₂—; —C₁₋₇alkylene-, —C₁₋₇alkylene-O—, —O—C₁₋₇alkylene-, —C₁₋₇alkylene-NR⁸⁰—, —NR⁸⁰—C₁₋₇alkylene-, —C₁₋₇alkylene-S—, —S—C₁₋₇alkylene-, —C₁₋₇alkylene-NR⁸⁰C(═O)—, —C(═O)NR⁸⁰C₁₋₇alkylene-, —C₁₋₇alkylene-C(═O)NR⁸⁰—, —NR⁸⁰C(═O)C₁₋₇alkylene-, —C₁₋₇alkylene-NR⁸⁰C(═O)NR⁸⁰—, —NR⁸⁰C(═O)NR⁸⁰C₁₋₇alkylene-, —C₁₋₇alkylene-SO₂NR⁸⁰—, —SO₂NR⁸⁰C₁₋₇alkylene-, —SO₂NR⁸⁰C(═O)—, —C(═O)NR⁸⁰SO₂—, NR⁸⁰SO₂NR⁸⁰C(═O)NR⁸⁰—, —NR⁸⁰C(═O)NR⁸⁰SO₂NR⁸⁰, —OC(═O)NR⁸⁰—, —NR⁸⁰C(═O)O—; —C₁₋₇alkylene-OC(═O)NR⁸⁰—, —NR⁸⁰C(═O)O—C₁₋₇alkylene-; —C₁₋₇alkylene-NR⁸⁰C(═O)O—, —OC(═O)NR⁸⁰—C₁₋₇alkylene-; —SO₂NR⁸⁰C₁₋₇alkylene- or —C₁₋₇alkylene-NR⁸⁰SO₂—;

B is optionally substituted C₁₋₇₀alkyl or C₁₋₇₀alkylene, wherein the C₁₋₇₀alkyl or C₁₋₇₀alkylene is optionally substituted with one or more functional groups selected from hydroxyl, oxo, carboxy, guanidino, amidino, —N(R⁸⁰)₂, —N(R⁸⁰)₃, phosphate, phosphonate, phospinate, sulfate, sulfonate and sulfinate, and wherein the C₁₋₇₀ alkyl or C₁₋₇₀alkylene optionally comprises one or more moieties selected from —NR⁸⁰—, —S—; —O—, —C₃₋₇cycloalkyl-, —C₃₋₇heterocyclyl-, —O₅₋₇heteroaryl-, —O₅₋₇aryl- and —SO₂—;

I is a compound of structure (I);

R⁸⁰ is independently, at each occurrence, hydrogen, C₁₋₇alkyl or —B-(L³-I)ₘ; and

m is an integer ranging from 0 to 10.

In some other embodiments of the foregoing compound, R³ is selected from: hydrogen, C₁₋₇-alkyl, halogen, C₁₋₇-haloalkyl, C₁₋₇-alkoxy, cyano, C₃₋₇-cycloalkyl, —O—C₃₋₇-cycloalkyl, —S(O)₀₋₂—C₁₋₇-alkyl, N-heterocyclyl, five-membered heteroaryl, phenyl and —NR¹⁵R¹⁶, wherein R¹⁵ and R¹⁶ are the same or different and independently selected from hydrogen, C₁₋₇-alkyl and C₃₋₇-cycloalkyl;

In some other embodiments of the foregoing compound, R⁸, R⁹, R¹⁰, R¹¹ and R¹² are the same or different and independently selected from: Q, hydrogen, C₁₋₇-alkyl, C₂₋₇-alkenyl, C₂₋₇-alkynyl, halogen, halogen-C₁₋₇-alkyl, C₁₋₇-alkoxy, halogen-C₁₋₇-alkoxy, hydroxy, hydroxy-C₁₋₇-alkoxy, hydroxy-C₁₋₇-alkyl, hydroxy-C₃₋₇-alkenyl, hydroxy-C₃₋₇-alkynyl, cyano, carboxyl, C₁₋₇-alkoxycarbonyl, amino carbonyl, carboxyl-C₁₋₇-alkyl, carboxyl-C₂₋₇-alkenyl, carboxyl-C₂₋₇-alkynyl, C₁₋₇-alkoxycarbonyl-C₁₋₇-alkyl, C₁₋₇-alkoxycarbonyl-C₂₋₇-alkenyl, C₁₋₇-alkoxycarbonyl-C₂₋₇-alkynyl, carboxyl-C₁₋₇-alkoxy, C₁₋₇-alkoxycarbonyl-C₁₋₇-alkoxy, carboxyl-C₁₋₇-alkyl-aminocarbonyl, carboxyl-C₁₋₇-alkyl-(C₁₋₇-alkylamino)-carbonyl, C₁₋₇-alkoxycarbonyl-C₁₋₇-alkyl-aminocarbonyl, C₁₋₇-alkoxycarbonyl-C₁₋₇-alkyl-(C₁₋₇-alkylamino)-carbonyl, carboxyl-C₁₋₇-alkyl-aminocarbonyl-C₁₋₇-alkyl, carboxyl-C₁₋₇-alkyl-(C₁₋₇-alkylamino)-carbonyl-C₁₋₇-alkyl, C₁₋₇-alkoxycarbonyl-C₁₋₇-alkyl-aminocarbonyl-C₁₋₇-alkyl, C₁₋₇-alkoxycarbonyl-C₁₋₇-alkyl-(C₁₋₇-alkylamino)-carbonyl-C₁₋₇-alkyl, hydroxy-C₁₋₇-alkyl-aminocarbonyl, di-(hydroxy-C₁₋₇-alkyl)aminocarbonyl, aminocarbonyl-C₁₋₇-alkyl-amino carbonyl, hydroxysulfonyl-C₁₋₇-alkyl-aminocarbonyl, hydroxysulfonyl-C₁₋₇-alkyl-(C₁₋₇-alkyl-amino)-carbonyl, di-(C₁₋₇-alkoxycarbonyl-C₁₋₇-alkyl)-methylaminocarbonyl, phenyl, wherein phenyl is unsubstituted or substituted by one to three groups selected from halogen, C₁₋₇-alkoxy, carboxyl or C₁₋₇-alkoxycarbonyl, phenyl-carbonyl, wherein phenyl is unsubstituted or substituted by one to three groups selected from halogen, C₁₋₇-alkoxy, carboxyl or C₁₋₇-alkoxycarbonyl, phenyl-aminocarbonyl, wherein phenyl is unsubstituted or substituted by one to three groups selected from halogen, C₁₋₇-alkoxy, carboxyl or C₁₋₇-alkoxycarbonyl, phenyl-C₁₋₇-alkyl, wherein phenyl is unsubstituted or substituted by one to three groups selected from halogen C₁₋₇-alkoxy, carboxyl or C₁₋₇-alkoxycarbonyl, phenyl-C₂₋₇-alkynyl, wherein phenyl is unsubstituted or substituted by one to three groups selected from halogen, C₁₋₇-alkoxy, carboxyl or C₁₋₇-alkoxycarbonyl, heteroaryl, wherein heteroaryl is unsubstituted or substituted by one to three groups selected from halogen C₁₋₇-alkoxy, carboxyl or C₁₋₇-alkoxycarbonyl, heteroaryl-carbonyl, wherein heteroaryl is unsubstituted or substituted by one to three groups selected from halogen, C₁₋₇-alkoxy, carboxyl or C₁₋₇-alkoxycarbonyl, heteroaryl-aminocarbonyl, wherein heteroaryl is unsubstituted or substituted by one to three groups selected from halogen, C₁₋₇-alkoxy, carboxyl or C₁₋₇-alkoxycarbonyl, heteroaryl-C₁₋₇-alkyl, wherein heteroaryl is unsubstituted or substituted by one to three groups selected from halogen, C₁₋₇-alkyl, C₁₋₇-alkoxy, carboxyl or C₁₋₇-alkoxycarbonyl, heteroaryl-C₁₋₇-alkyl-aminocarbonyl, wherein heteroaryl is unsubstituted or substituted by one to three groups selected from halogen, C₁₋₇-alkoxy, carboxyl or C₁₋₇-alkoxycarbonyl, and heteroaryl-carbonyl-C₁₋₇-alkyl, wherein heteroaryl is unsubstituted or substituted by one to three groups selected from halogen, C₁₋₇-alkoxy, carboxyl or C₁₋₇-alkoxycarbonyl.

As one of skill in the art will appreciate, each of the substituents of compounds as described herein may also be optionally substituted with one or more of the substituents defined above and below.

In some other embodiments of the foregoing compound, X is CR⁵⁰R⁵¹ and the compound has the following structure (II):

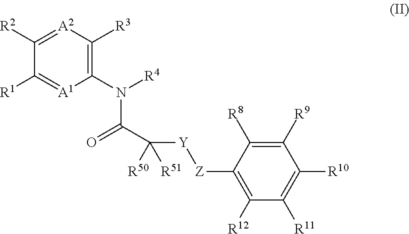

In other embodiments, Y is O and Z is CR⁷⁰R⁷¹ and the compound has the following structure (III):

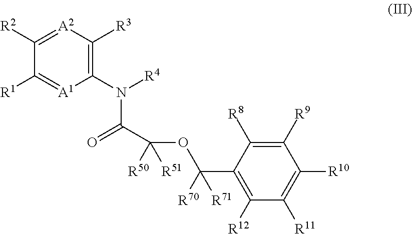

In yet other embodiments, Y is NR⁶² and Z is CR⁷⁰R⁷¹ and the compound has the following structure (IV):

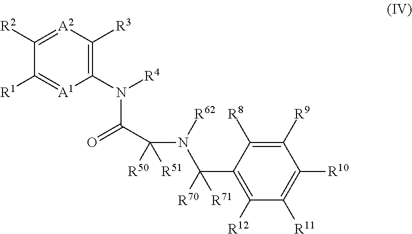

In some other embodiments, Y is CR⁶⁰R⁶¹ and Z is O and the compound has the following structure (V):

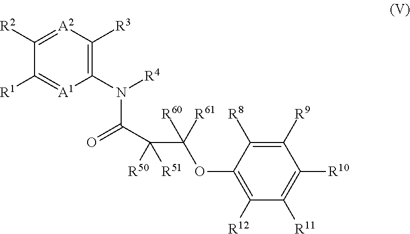

In even other embodiments, R⁵⁰ and R⁵¹ taken together with the C atom to which they are attached form a cycloalkyl or heterocyclyl, wherein the cycloalkyl or heterocyclyl are optionally substituted by one or two groups selected from halogen, hydroxy, oxo, C₁₋₇-alkyl, C₁₋₇-alkylcarbonyl, C₁₋₇-alkyloxycarbonyl, C₁₋₇-alkoxy, C₁₋₇-alkoxyalkyl and C₁₋₇-alkyl-S(O)₀₋₂—, wherein the compound has the following structure (VI) and wherein W represents the cycloalkyl or heterocycly group:

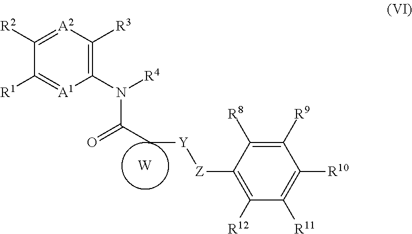

In even other embodiments, Y is O and Z is CR⁷⁰R⁷¹ and the compound has the following structure (VII):

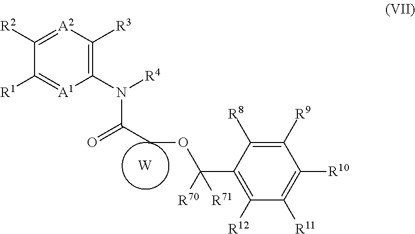

In still other embodiments, Y is NR⁶² and Z is CR⁷⁰R⁷¹ and the compound has the following structure (VIII):

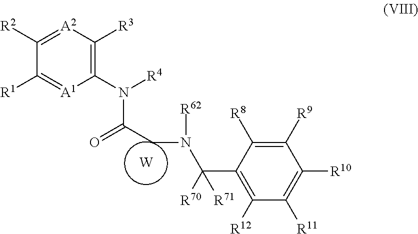

In other embodiments, Y is CR⁶⁰R⁶¹ and Z is O and the compound has the following structure (IX):

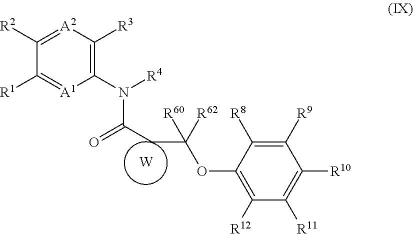

In still other embodiments, the compound has one of the following structures (VIa), (VIb), (VIc), (VId), (VIe), (VIf), (VIg) or (VIh):

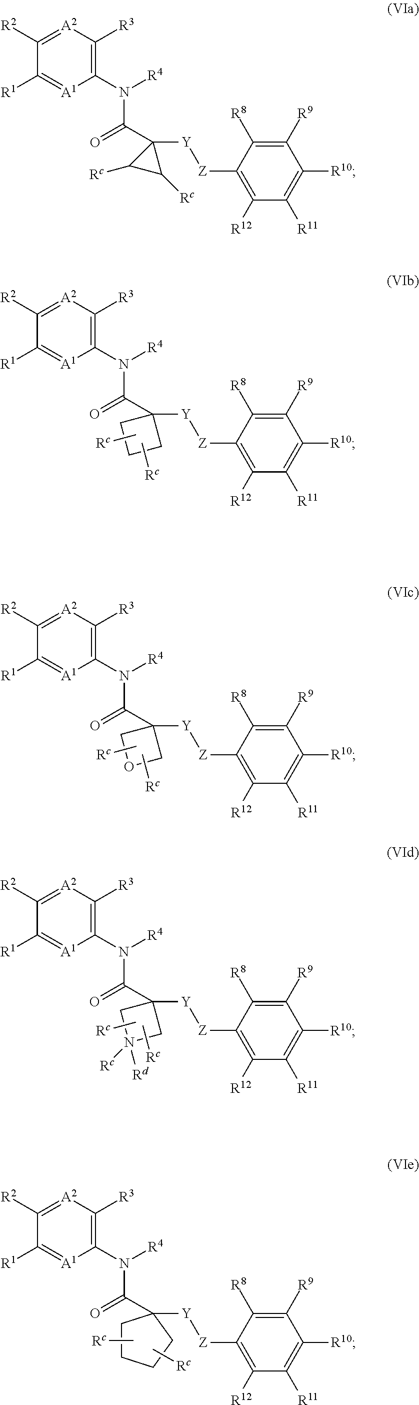

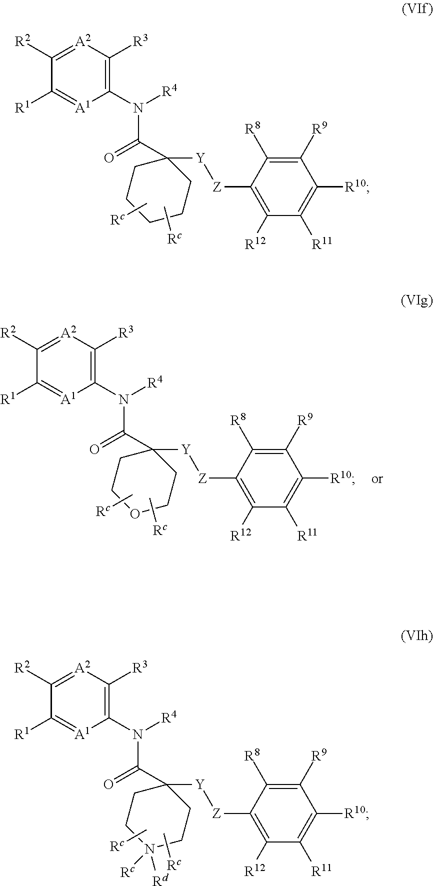

wherein:
R^{c} is independently, at each occurrence, hydrogen, halogen, hydroxy, oxo, C₁₋₇-alkyl, C₁₋₇-alkylcarbonyl, C₁₋₇-alkyloxycarbonyl, C₁₋₇-alkoxy, C₁₋₇-alkoxyalkyl or C₁₋₇-alkyl-S(O)₀₋₂—; and
R^{d} is independently, at each occurrence, an electron pair, hydrogen, C₁₋₇-alkyl, C₁₋₇-alkylcarbonyl, C₁₋₇-alkyloxycarbonyl, C₁₋₇-alkoxyalkyl or C₁₋₇-alkyl-S(O)₀₋₂—. For example, in some embodiments Y is O and Z is CR⁷⁰R⁷¹. In other embodiments Y is NR⁶² and Z is CR⁷⁰R⁷¹, and in other embodiments Y is CR⁶⁰R⁶¹ and Z is O.

In even other embodiments, X and Y taken together form a cycloalkyl, heterocyclyl, aryl or heteroaryl, wherein the cycloalkyl, heterocyclyl, aryl or heteroaryl are optionally substituted by one or two groups selected from halogen, hydroxy, oxo, C₁₋₇-alkyl, C₁₋₇-alkylcarbonyl, C₁₋₇-alkyloxycarbonyl, C₁₋₇-alkoxy, C₁₋₇-alkoxyalkyl, (R^{a})₂(R^{b})N— and C₁₋₇-alkyl-S(O)₀₋₂—, wherein R^{a} is independently, at each occurrence, hydrogen or C₁₋₇-alkyl and R^{b} is an electron pair, hydrogen or C₁₋₇-alkyl, and provided that when X and Y form phenyl, pyridyl, pyridyl-N-oxide or pyrimidinyl then Z is not O, wherein the compound has the following structure (X), and wherein V represents the cycloalkyl, heterocyclyl, aryl or heteroaryl:

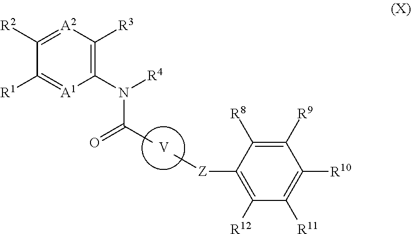

In some embodiments of the compound of structure (X), Z is CR⁷⁰R⁷¹ and the compound has the following structure (XI):

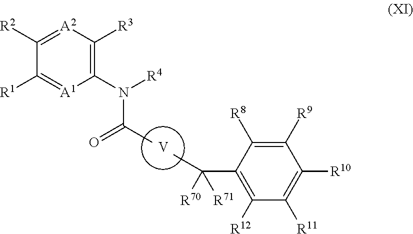

In other embodiments of the compound of structure (X), Z is CR⁷⁰R⁷¹ and R⁷⁰ and R⁷¹ taken together form oxo (═O) and the compound has the following structure (XII):

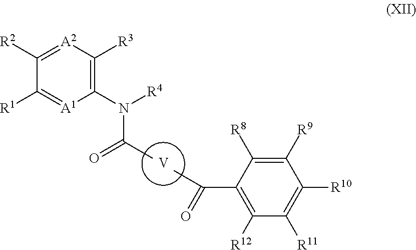

In still other embodiments of the compound of structure (X), Z is O and the compound has the following structure (XIII):

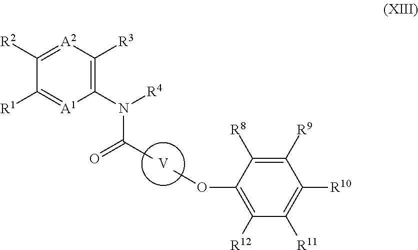

In even more embodiments of the compound of structure (X), Z is S(O)₀₋₂ and the compound has the following structure (XIV):

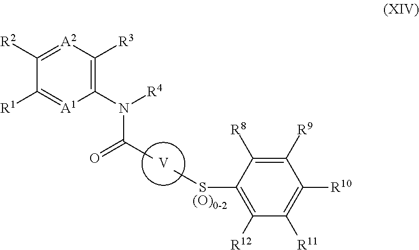

For example, in some embodiments of the compounds of structure (XIV), Z is —SO₂—.

In even more embodiments of the compound of structure (X), the compound has one of the following structures (Xa), (Xb), (Xc), (Xd), (Xe), (Xf), (Xg), (Xh), (Xi), (Xj), (Xk), (Xl), (Xm), (Xn), (Xo), (Xp), (Xq), (Xr) or (Xs):

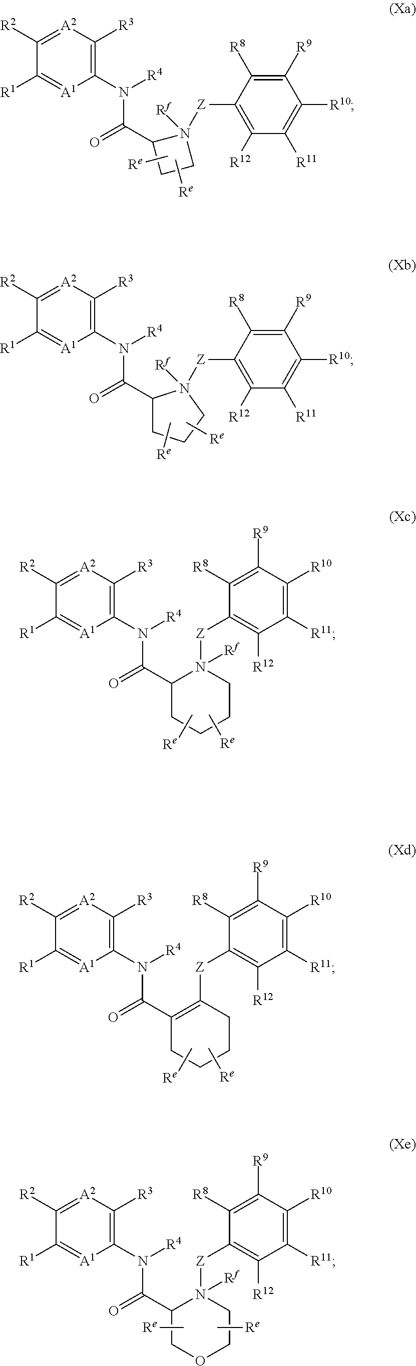

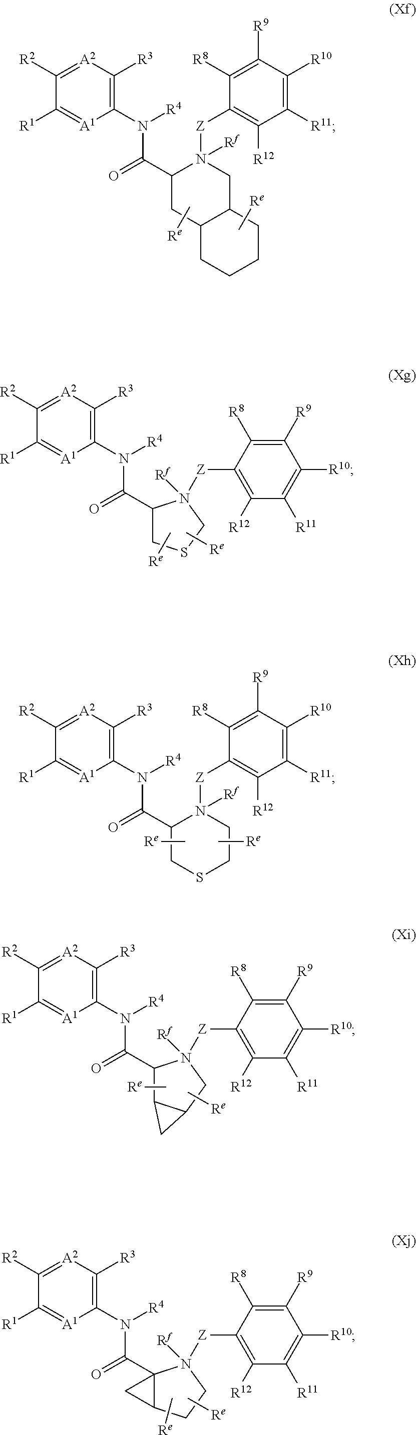

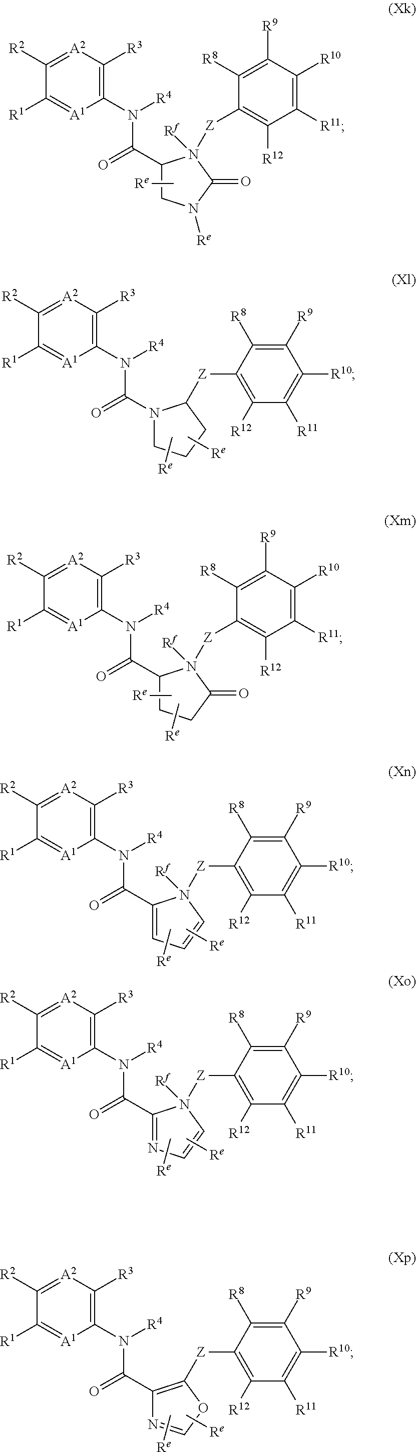

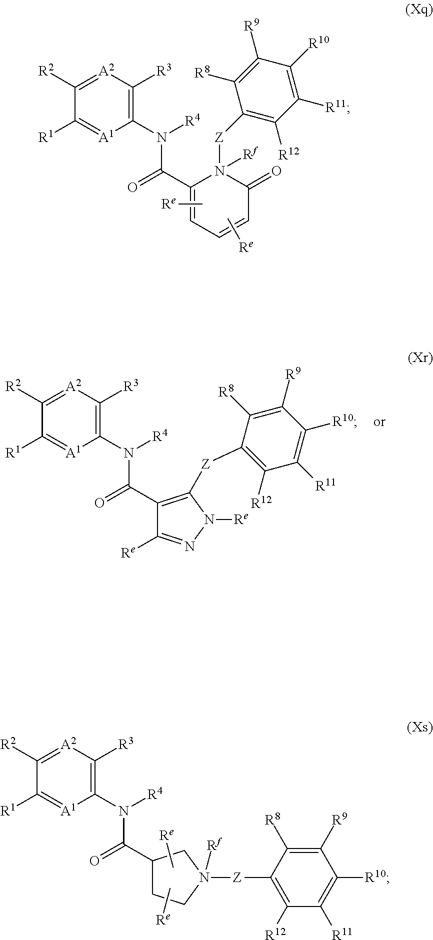

wherein:
R^{e} is independently, at each occurrence, hydrogen, halogen, hydroxy, oxo, C₁₋₇-alkyl, C₁₋₇-haloalkyl, C₁₋₇-alkylcarbonyl, C₁₋₇-alkyloxycarbonyl, C₁₋₇-alkoxy, C₁₋₇-alkoxyalkyl, (R^{a})₂(R^{b})N— and C₁₋₇-alkyl-S(O)₀₋₂—; and
R^{f} is an electron pair, hydrogen or C₁₋₇-alkyl.

For example, in certain embodiments of the foregoing Z is CR⁷⁰R⁷¹. In other embodiments, Z is CR⁷⁰R⁷¹ and R⁷⁰ and R⁷¹ taken together form oxo (═O). In still other embodiments Z is O. In yet more embodiments Z is —S(O)₀₋₂—, for example in some embodiments Z is —SO₂—.

In some other embodiments, Y is absent and Z is O and the compound has the following structure (XV):

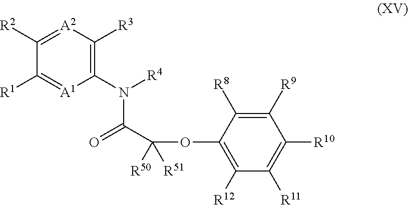

In some embodiments of the compound of structure (XV), R⁵⁰ and R⁵¹ taken together with the C atom to which they are attached form a cycloalkyl or heterocyclyl, wherein the cycloalkyl or heterocyclyl are optionally substituted by one or two groups selected from halogen, hydroxy, oxo, C₁₋₇-alkyl, C₁₋₇-alkylcarbonyl, C₁₋₇-alkyloxycarbonyl, C₁₋₇-alkoxy, C₁₋₇-alkoxyalkyl and C₁₋₇-alkyl-S(O)₀₋₂—, wherein the compound has the following structure (XVI) and wherein W represents the cycloalkyl or heterocyclyl group:

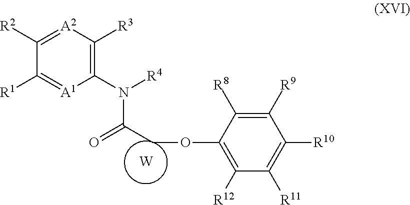

In still other embodiments, A1 and A2 are both CR¹³. For example, in some embodiments of the foregoing R¹³ is hydrogen.

In other embodiments, R³ and R⁴ together are -L-(CR¹⁷R¹⁸)ₙ— and form part of a ring. For example, in some embodiments the compound has the following structure (XVII):

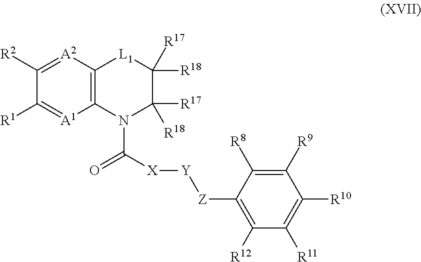

In some embodiments of the compound of structure (XVII), L¹ is —C(═O)—, —S—, —S(O)₂— or —N(R²¹)—. For example, in some embodiments R²¹ is C₃₋₇-cycloalkyl.

In other embodiments of the compound of structure (XVII), the compound has one of the following structures (XVIIa), (XVIIb), (XVIIc) or (XVIId):

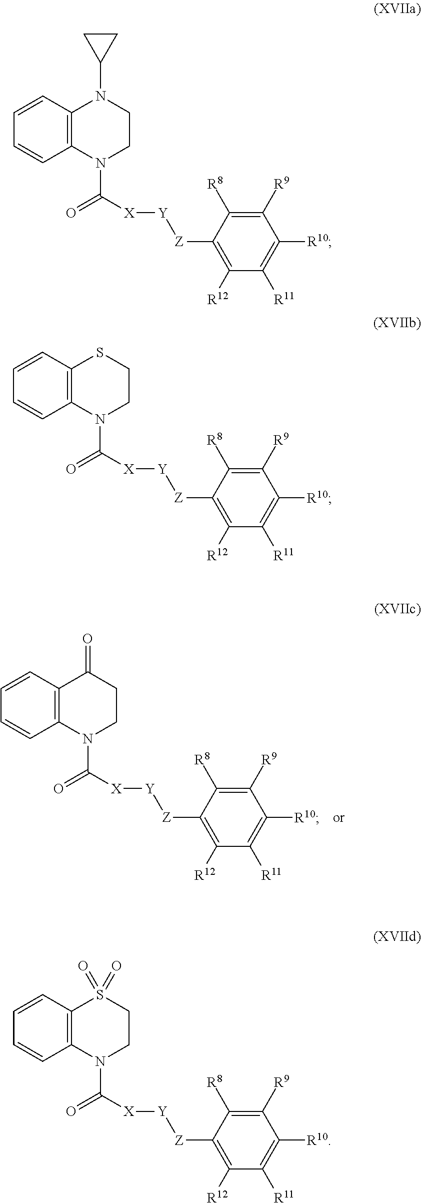

In other embodiments of the compound of structure XVIIa, X is CR⁵⁰R⁵¹. R⁵⁰ and R⁵¹ taken together with the C atom to which they are attached form a cycloalkyl or heterocyclyl, wherein the cycloalkyl or heterocyclyl are optionally substituted by one or two groups selected from halogen, hydroxy, oxo, C₁₋₇-alkyl, C₁₋₇-alkylcarbonyl, C₁₋₇-alkyloxycarbonyl, C₁₋₇-alkoxy, C₁₋₇-alkoxyalkyl and C₁₋₇-alkyl-S(O)₀₋₂—. In still other embodiments, R⁵⁰ and R⁵¹ taken together with the C atom to which they are attached form a cycloalkyl according to structure (VIa), and the compound of structure (XXVIIa) has the following structure (XVIIa-1):

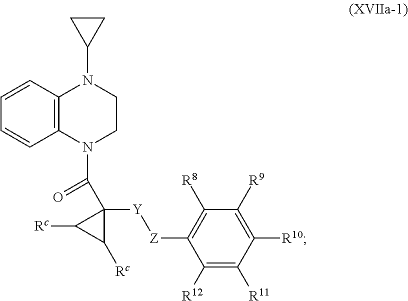

wherein:

R^{c} is independently, at each occurrence, hydrogen, halogen, hydroxy, oxo, C₁₋₇-alkyl, C₁₋₇-alkylcarbonyl, C₁₋₇-alkyloxycarbonyl, C₁₋₇-alkoxy, C₁₋₇-alkoxyalkyl or C₁₋₇-alkyl-S(O)₀₋₂—. In some other embodiments, Re is hydrogen.

For example, in certain embodiments of a compound of formula (XVIIa-1), Y is O and Z is CR⁷⁰R⁷¹. In other embodiments, Y is O and Z is CR⁷⁰R⁷¹. In other embodiments, Y is NR⁶² and Z is CR⁷⁰R⁷¹. In other embodiments, Y is NR⁶² and Z is O. In other embodiments, Y is NR⁶² and Z is S(O)₀₋₂. In other embodiments, Y is CR⁶⁰R⁶¹ and Z is CR⁷⁰R⁷¹. In other embodiments, Y is CR⁶⁰R⁶¹ and Z is O. In other embodiments, Y is CR⁶⁰R⁶¹ and Z is S(O)₀₋₂.

In other embodiments of the compound of structure XVIIa, X and Y taken together form a cycloalkyl, heterocyclyl, aryl or heteroaryl, wherein the cycloalkyl, heterocyclyl, aryl or heteroaryl are optionally substituted by one or two groups selected from halogen, hydroxy, oxo, C₁₋₇-alkyl, C₁₋₇-alkylcarbonyl, C₁₋₇-alkyloxycarbonyl, C₁₋₇-alkoxy, C₁₋₇-alkoxyalkyl, (R^{a})₂(R^{b})N— and C₁₋₇-alkyl-S(O)₀₋₂—, wherein R^{a} is independently, at each occurrence, hydrogen or C₁₋₇-alkyl and R^{b} is an electron pair, hydrogen or C₁₋₇-alkyl, and provided that when X and Y form phenyl, pyridyl, pyridyl-N-oxide or pyrimidinyl then Z is not O. In still other embodiments, X and Y taken together form a heterocyclyl according structure (Xb) or structure (Xg), and the compound of structure (XXVIIa) has the following structure (XVIIa-2) or structure (XVIIa-3), respectively:

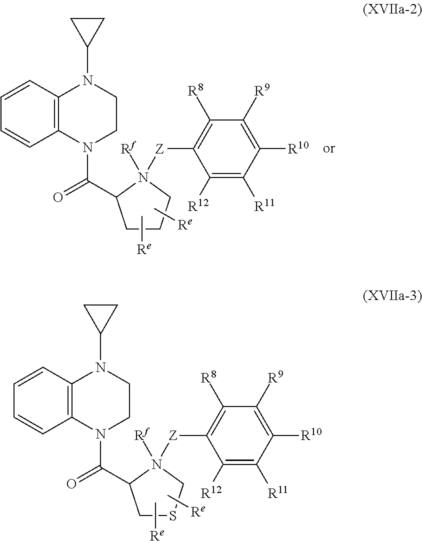

wherein:

R^{e} is independently, at each occurrence, hydrogen, halogen, hydroxy, oxo, C₁₋₇-alkyl, C₁₋₇-haloalkyl, C₁₋₇-alkylcarbonyl, C₁₋₇-alkyloxycarbonyl, C₁₋₇-alkoxy, C₁₋₇-alkoxyalkyl, (R^{a})₂(R^{b})N— and C₁₋₇-alkyl-S(O)₀₋₂—; and R^{f} is an electron pair, hydrogen or

C₁₋₇-alkyl.

For example, in certain embodiments of the foregoing Z is CR⁷⁰R⁷¹. In other embodiments, Z is CR⁷⁰R⁷¹ and R⁷⁰ and R⁷¹ taken together form oxo (═O). In still other embodiments Z is O. In yet more embodiments Z is —S(O)₀₋₂—, for example in some embodiments Z is —SO₂—.

In still other embodiments, the compound has the following structure (XVIII):

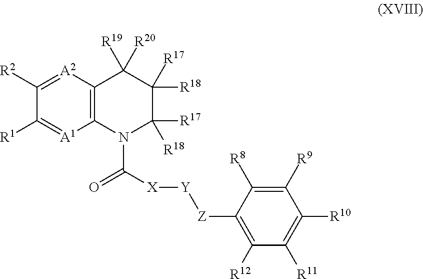

In other embodiments of the compound of structure (XVIII), R²⁰ is N(R²¹)₂. For example, in some embodiments the compound has one of the following structures (XVIIIa), (XVIIIb), (XVIIIc), (XVIIId), (XVIIIe), (XVIIIf), (XVIIIg), (XVIIIh), (XVIIIi), (XVIIIj), (XVIIIk) or (XVIIIl):

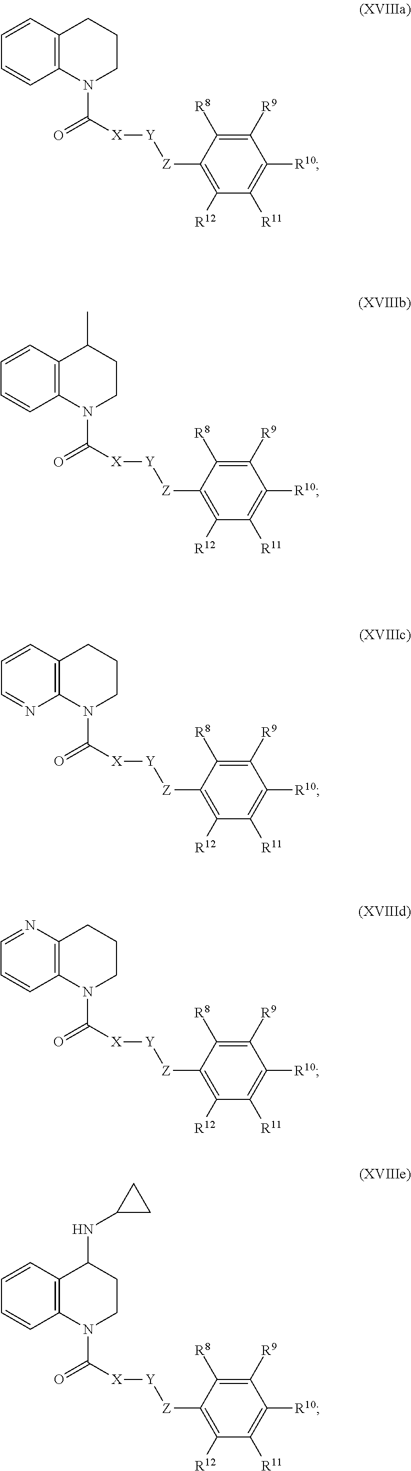

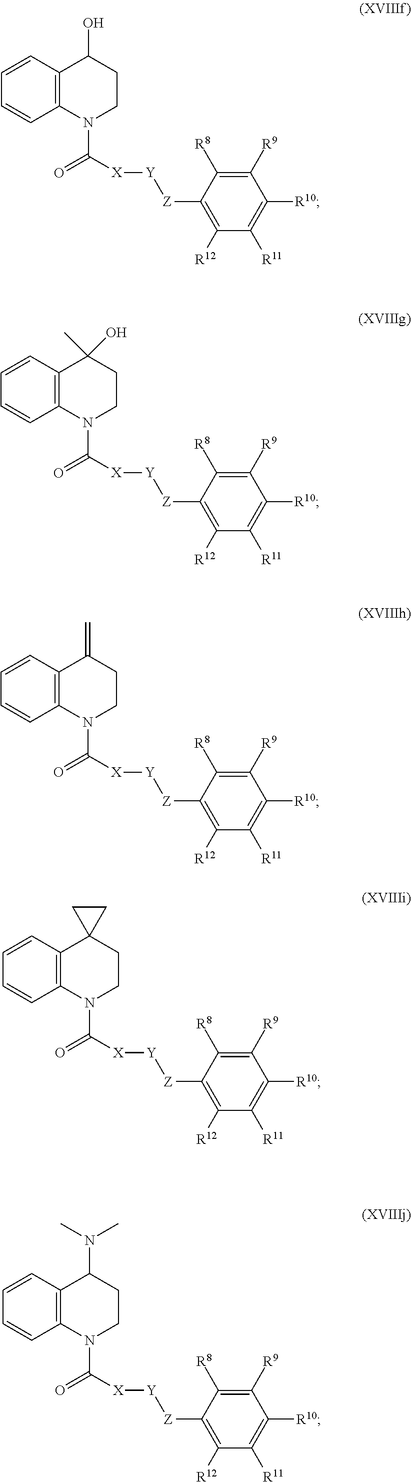

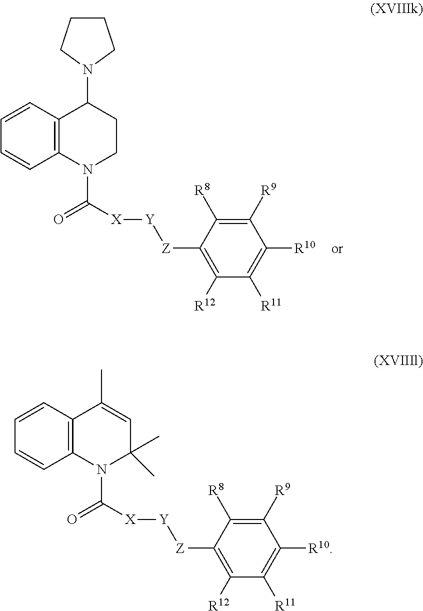

In still other embodiments, A¹ and A² are each independently CH or N and R³ is C₁₋₇-alkoxy, —O—C₃₋₇-cycloalkyl, or —O—C₁₋₇-alkyl-C₃₋₇-cycloalkyl. For example, in some embodiments the compound has one of the following structures (XIXa), (XIXb), (XIXc), (XIXd), (XIXe), (XIXf) or (XIXg):

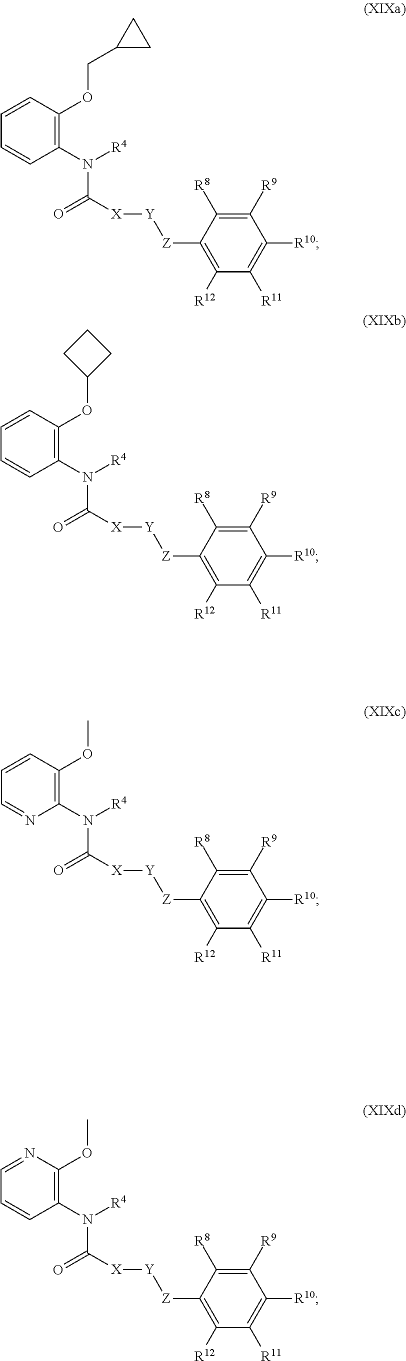

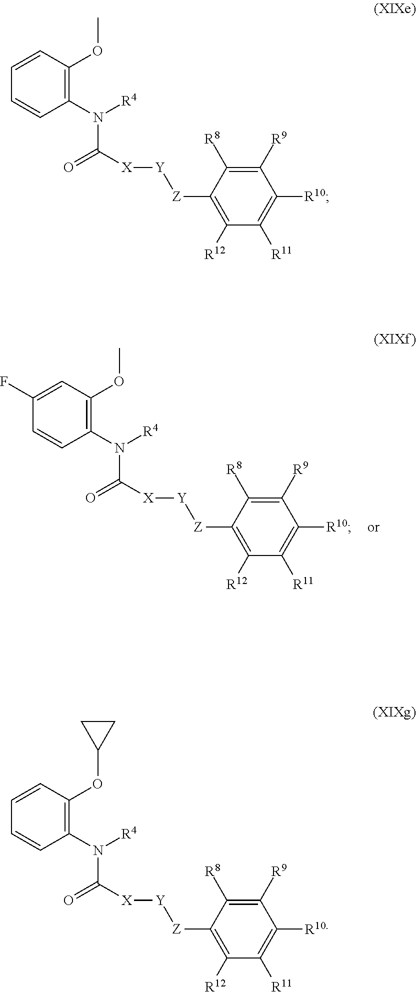

In other embodiments, the compound has the structure (XIXg).

In certain embodiments of the foregoing, X is CR⁵⁰R⁵¹. In other embodiments of the compound of structure (XIXg), R⁵⁰ and R⁵¹ taken together with the C atom to which they are attached form a cycloalkyl or heterocyclyl, wherein the cycloalkyl or heterocyclyl are optionally substituted by one or two groups selected from halogen, hydroxy, oxo, C₁₋₇-alkyl, C₁₋₇-alkylcarbonyl, C₁₋₇-alkyloxycarbonyl, C₁₋₇-alkoxy, C₁₋₇-alkoxyalkyl and C₁₋₇-alkyl-S(O)₀₋₂—, For example, in certain embodiments, R⁵⁰ and R⁵¹ taken together with the C atom to which they are attached form a cyclopropyl.

In still other embodiments, Y is O and Z is CR⁷⁰R⁷¹; Y is NR⁶² and Z is CR⁷⁰R⁷¹; Y is NR⁶² and Z is O; Y is NR⁶² and Z is S(O)₀₋₂; Y is CR⁶⁰R⁶¹ and Z is CR⁷⁰R⁷¹; Y is cR⁶⁰R⁶¹ and Z is O; and Y is CR⁶⁰R⁶¹ and Z is S(O)₀₋₂.

In certain embodiments of the foregoing, X and Y taken together form a cycloalkyl, heterocyclyl, aryl or heteroaryl, wherein the cycloalkyl, heterocyclyl, aryl or heteroaryl are optionally substituted by one or two groups selected from halogen, hydroxy, oxo, C₁₋₇-alkylcarbonyl, C₁₋₇-alkyloxycarbonyl, C₁₋₇-alkoxy, C₁₋₇-alkoxyalkyl, (R^{a})₂(R^{b})N— and C₁₋₇-alkyl-S(O)₀₋₂—, wherein R^{a} is independently, at each occurrence, hydrogen or C₁₋₇-alkyl and R^{b} is an electron pair, hydrogen or C₁₋₇-alkyl, and provided that when X and Y form phenyl, pyridyl, pyridyl-N-oxide or pyrimidinyl then Z is not O. For example, in certain embodiments, X and Y taken together form a heterocyclyl. In still other embodiments, the heterocyclyl is pyrrolidinyl or thiazolidinyl.

For example, in certain embodiments of the foregoing Z is CR⁷⁰R⁷¹. In other embodiments, Z is CR⁷⁰R⁷¹ and R⁷⁰ and R⁷¹ taken together form oxo (═O). In still other embodiments Z is O. In yet more embodiments Z is —S(O)₀₋₂—, for example in some embodiments Z is —SO₂—.

In even more embodiments, A¹ is CR¹³ and A² is CR¹⁴ and wherein R¹³ and R¹⁴ are independently from each other selected from hydrogen, halogen, halogen-C₁₋₇-alkyl and C₁₋₇-alkoxy.

In other embodiments, A¹ is CR¹³ and A² is N, with R¹³ being independently from each other selected from hydrogen, halogen, halogen-C₁₋₇-alkyl and C₁₋₇-alkoxy.

In yet more embodiments, R¹ and R² are independently from each other selected from the group consisting of hydrogen, halogen and halogen-C₁₋₇-alkyl.

In other embodiments, R³ and R⁴ together are -L¹-(CR¹⁷R¹⁸)ₙ— and form part of a ring; wherein

L¹ is selected from —CR¹⁹R²⁰- and —NR²¹—;

R¹⁷ and R¹⁸ are independently from each other selected from hydrogen and C₁₋₇-alkyl;

R¹⁹ and R²⁰ are independently from each other selected from hydrogen, C₁₋₇-alkoxycarbonyl, unsubstituted heterocyclyl and heterocyclyl substituted by one or two groups selected from C₁₋₇-alkyl and halogen;

or R¹⁹ and R²⁰ together with the C atom to which they are attached form a cyclopropyl or oxetanyl ring or together form a ═CH₂ or ═CF₂ group;

R²¹ is selected from hydrogen, C₁₋₇-alkyl, halogen-C₁₋₇-alkyl, C₃₋₇-cycloalkyl and C₃₋₇-cycloalkyl-C₁₋₇-alkyl, wherein C₃₋₇-cycloalkyl is unsubstituted or substituted by carboxyl-C₁₋₇-alkyl or C₁₋₇-alkoxycarbonyl, heterocyclyl, heterocyclyl-C₁₋₇-alkyl, heteroaryl, heteroaryl-C₁₋₇-alkyl, carboxyl-C₁₋₇-alkyl, C₁₋₇-alkoxycarbonyl-C₁₋₇-alkyl, C₁₋₇-alkylcarbonyloxy-C₁₋₇-alkyl, C₁₋₇-alkylsulfonyl, phenyl, wherein phenyl is unsubstituted or substituted by carboxyl-C₁₋₇-alkyl or C₁₋₇-alkoxycarbonyl, phenylcarbonyl, wherein phenyl is unsubstituted or substituted by carboxyl-C₁₋₇-alkyl or C₁₋₇-alkoxycarbonyl, and phenylsulfonyl, wherein phenyl is unsubstituted or substituted by carboxyl-C₁₋₇-alkyl or C₁₋₇-alkoxycarbonyl;

or R²¹ and a R¹⁷ together are —(CH₂)₃— and form part of a ring, or R²¹ together with a pair of R¹⁷ and R¹⁸ are —CH═CH—CH═ and form part of a ring; and

n is 1, 2or 3.

In still other embodiments, the compound has structure (I), wherein:

L¹ is —NR²¹—,

R²¹ is selected from hydrogen, C₁₋₇-alkyl, C₃₋₇-cycloalkyl and C₃₋₇-cycloalkyl-C₁₋₇-alkyl, wherein C₃₋₇-cycloalkyl is unsubstituted or substituted by carboxyl-C₁₋₇-alkyl or C₁₋₇-alkoxycarbonyl, and C₁₋₇-alkylsulfonyl;

R¹⁷ and R¹⁸ are independently from each other selected from hydrogen and methyl; and

n is 2.

In still other embodiments, L¹ is —CH₂—, R¹⁷ and R¹⁸ are independently from each other selected from hydrogen and methyl and n is 2.

In other embodiments, R³ and R¹⁴ together are -L¹-(CR¹⁷R¹⁸)n- and form part of a ring; wherein L¹ is —NR²¹— or —O—, R²¹ is selected from hydrogen, C₁₋₇-alkyl and C₃₋₇-cycloalkyl, R¹⁷ and R¹⁸ are independently from each other selected from hydrogen and methyl, and n is 2.

For example, in some embodiments, L¹ is —O— and the compound has the following structure (XV):

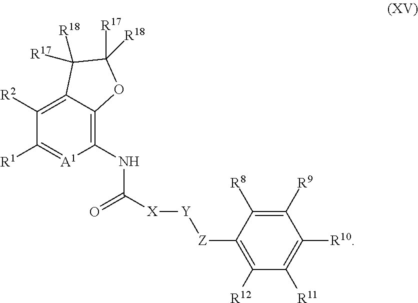

In certain embodiments of the foregoing structure (XV), R¹⁷ and R¹⁸ are hydrogen. In other embodiments, X is CR⁵⁰R⁵¹. In other embodiments of the compound of structure (XV), R⁵⁰ and R⁵¹ taken together with the C atom to which they are attached form a cycloalkyl or heterocyclyl, wherein the cycloalkyl or heterocyclyl are optionally substituted by one or two groups selected from halogen, hydroxy, oxo, C₁₋₇-alkyl, C₁₋₇-alkylcarbonyl, C₁₋₇-alkyloxycarbonyl, C₁₋₇-alkoxy, C₁₋₇-alkoxyalkyl and C₁₋₇-alkyl-S(O)₀₋₂—, For example, in certain embodiments, R⁵⁰ and R⁵¹ taken together with the C atom to which they are attached form a cyclopropyl.

In still other embodiments, Y is O and Z is CR⁷⁰R⁷¹; Y is NR⁶² and Z is CR⁷⁰R⁷¹; Y is NR⁶² and Z is O; Y is NR⁶² and Z is S(O)₀₋₂; Y is CR⁶⁰R⁶¹ and Z is CR⁷⁰R⁷¹; Y is cR⁶⁰R⁶¹ and Z is O; or Y is CR⁶⁰R⁶¹ and Z is S(O)₀₋₂.

In certain embodiments of the foregoing, X and Y taken together form a cycloalkyl, heterocyclyl, aryl or heteroaryl, wherein the cycloalkyl, heterocyclyl, aryl or heteroaryl are optionally substituted by one or two groups selected from halogen, hydroxy, oxo, C₁₋₇-alkyl, C₁₋₇-alkylcarbonyl, C₁₋₇-alkyloxycarbonyl, C₁₋₇-alkoxy, C₁₋₇-alkoxyalkyl, (R^{a})₂ (R^{b})N— and C₁₋₇-alkyl-S(O)₀₋₂—, wherein R^{a} is independently, at each occurrence, hydrogen or C₁₋₇-alkyl and R^{b} is an electron pair, hydrogen or C₁₋₇-alkyl, and provided that when X and Y form phenyl, pyridyl, pyridyl-N-oxide or pyrimidinyl then Z is not O. For example, in certain embodiments, X and Y taken together form a heterocyclyl. In still other embodiments, the heterocyclyl is pyrrolidinyl or thiazolidinyl.

For example, in certain embodiments of the foregoing Z is CR⁷⁰R⁷¹. In other embodiments, Z is CR⁷⁰R⁷¹ and R⁷⁰ and R⁷¹ taken together form oxo (═O). In still other embodiments Z is O. In yet more embodiments Z is —S(O)₀₋₂—, for example in some embodiments Z is —SO₂—.

In other embodiments, R³ is selected from hydrogen, C₁₋₇-alkyl, C₁₋₇-alkoxy, N-heterocyclyl and —NR¹⁵R¹⁶, wherein R¹⁵ and R¹⁶ are independently from each other selected from hydrogen, C₁₋₇-alkyl and C₃₋₇-cycloalkyl, and R⁴ is hydrogen or methyl.

In other embodiments, at least one of R⁸, R⁹, R¹⁰, R¹¹ or R¹² is halogen, C₁₋₇-alkyl, halogen-C₁₋₇-alkyl, C₁₋₇-alkoxy, halogen-C₁₋₇-alkoxy or cyano. For example, in some embodiments the halogen is chloro. In other embodiments, the other ones of R⁸, R⁹, R¹⁰, R¹¹ or R¹² are hydrogen.

In even more embodiments, the compound has one of the following structures (XXa), (XXb), (XXc), (XXd), (XXe), (XXf), (XXg), (XXh), (XXi), (XXj), (XXk) or (XXl):

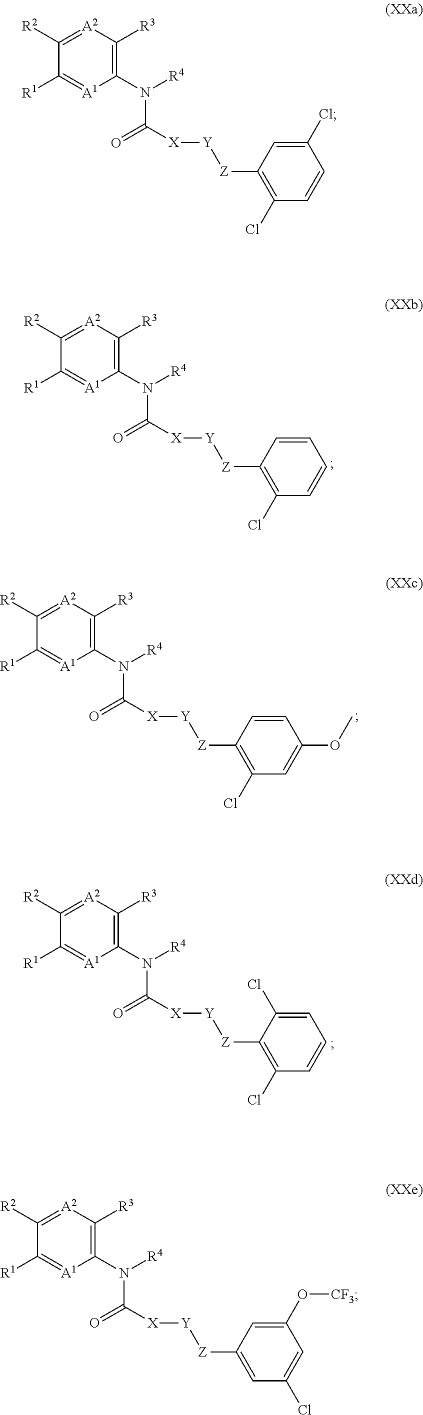

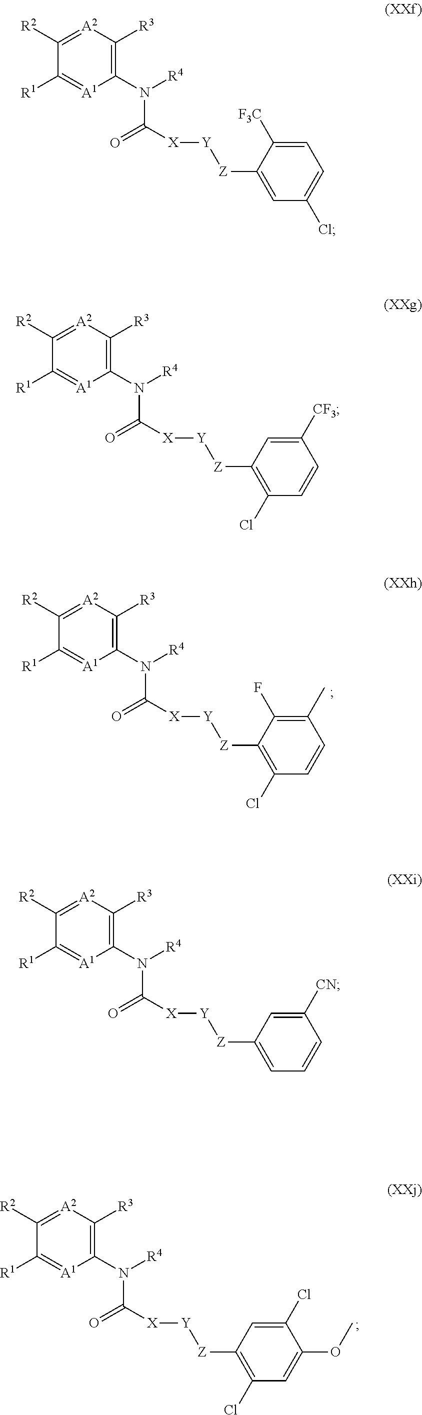

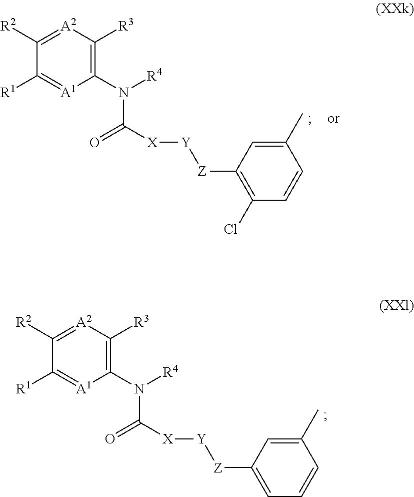

In still other embodiments, at least one of R⁸, R⁹, R¹⁰, R¹¹ or R¹² is Q. For example, in some embodiments R⁹ or R¹⁰ is Q. In other embodiments, the other ones of R⁸, R⁹, R¹⁰, R¹¹ or R¹² are selected from the group consisting of hydrogen, halogen, C₁₋₇-alkyl, halogen-C₁₋₇-alkyl, C₁₋₇-alkoxy, halogen-C₁₋₇-alkoxy and cyano.

In other embodiments, the compound has one of the following structures (XXIa), (XXIb), (XXIc), (XXId), (XXIe), (XXIf), (XXIg), (XXIh), (XXIi), (XXIj), (XXIk) or (XXIl):

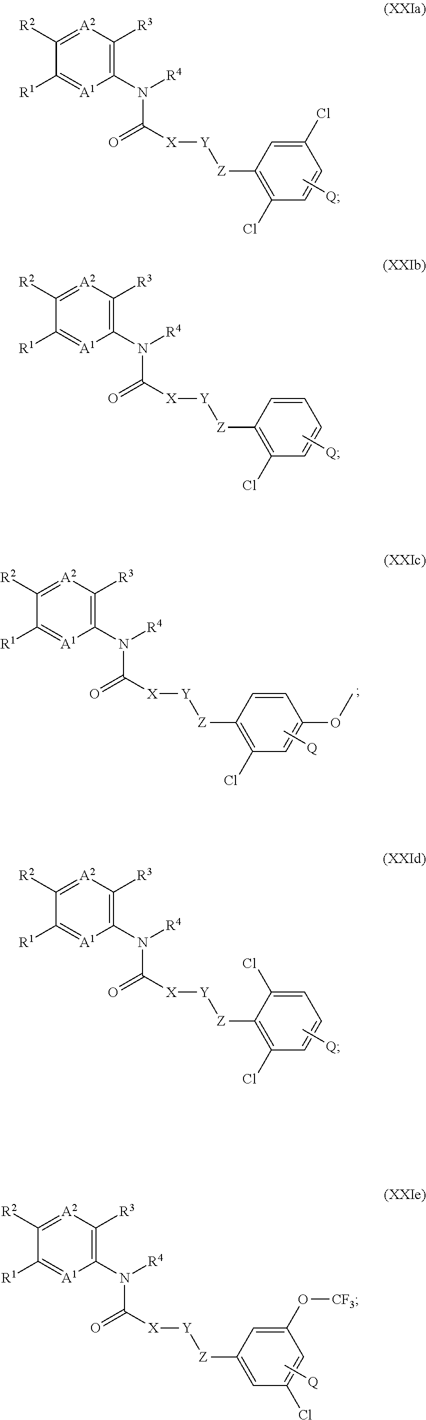

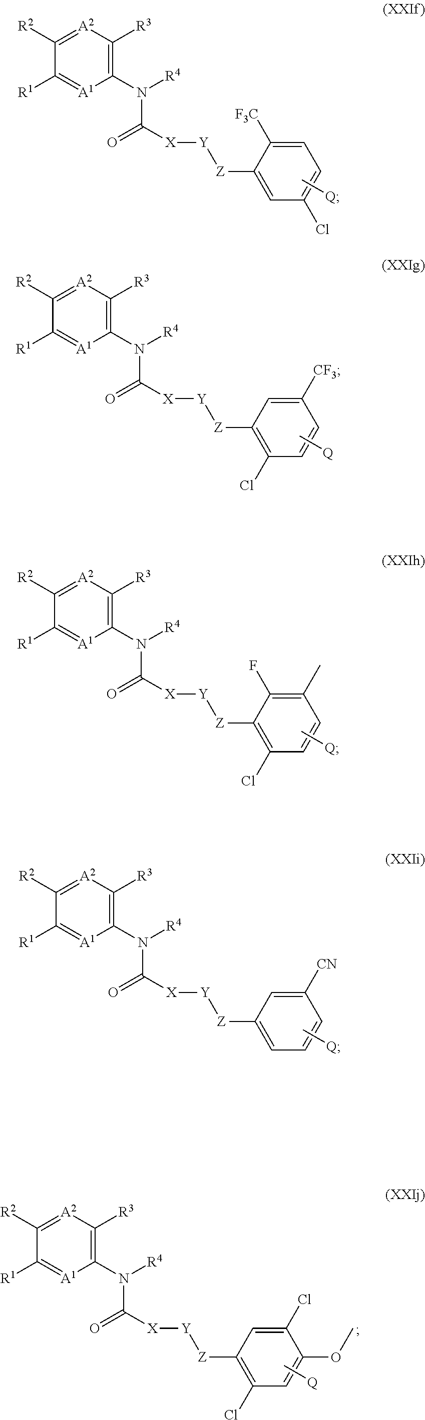

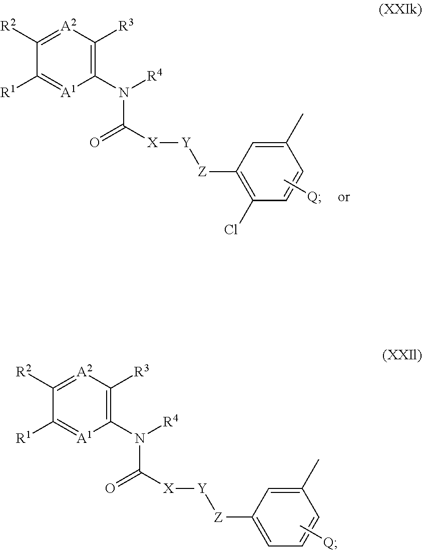

In even more embodiments, L² is —O—, —C₁₋₇ alkylene-; —C₁₋₇alkylene-NR⁸⁰—, —C₁₋₇alkylene-NR⁸⁰C(═O)—, —C₁₋₇alkylene-C(═O)NR⁸⁰— or —C₁₋₇alkylene-NR⁸⁰C(═O)NR⁸⁰—.

In still other embodiments, Q is -L²CR⁸¹R⁸²(CR⁸³R⁸⁴)ₘ₁G, wherein:

R⁸¹, R⁸², R⁸³ and R⁸⁴ are independently, at each occurrence, hydrogen or hydroxyl;

G is —CH₃, —CH₂OH, —CO₂H or -L³-I; and ml is an integer ranging from 1 to 21.

In still other embodiments, G is —CH₃, —CH₂OH, or —CO₂H.

For example, in some embodiments of the foregoing, for each occurrence of R⁸³ and R⁸⁴, one of R⁸³ or R⁸⁴ is hydrogen and the other of R⁸³ or R⁸⁴ is hydroxyl.

In other embodiments, Q has one of the following structures (XXIIa), (XXIIb), (XXIIc), (XXIId), (XXIIe), (XXIIf), (XXIIg), (XXIIh), (XXIIi), (XXIIj), (XXIIk), (XXIIl), (XXIIm), (XXIIn), (XXIIo) or (XXIIp):

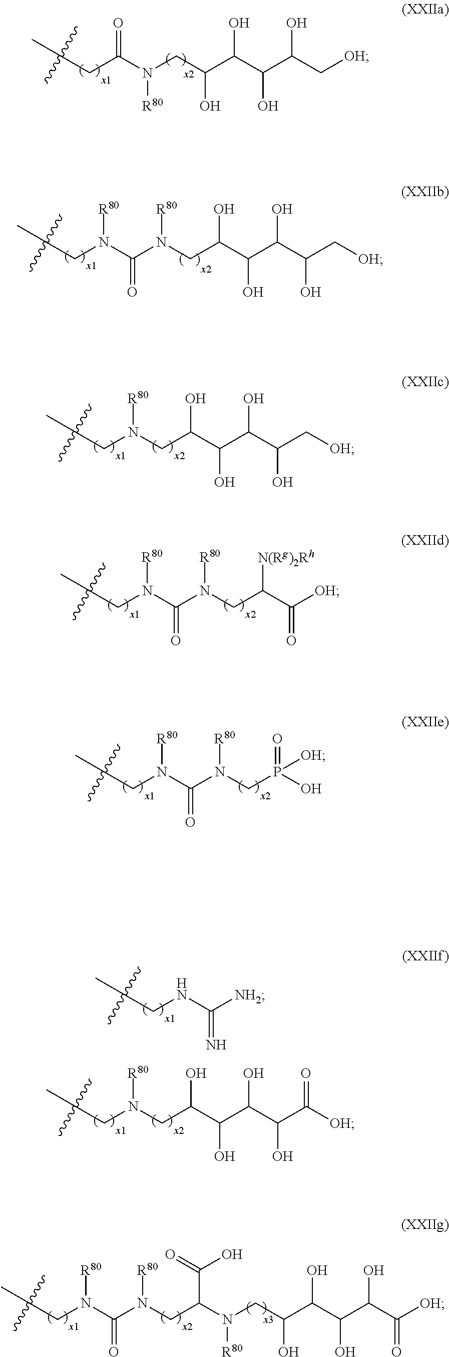

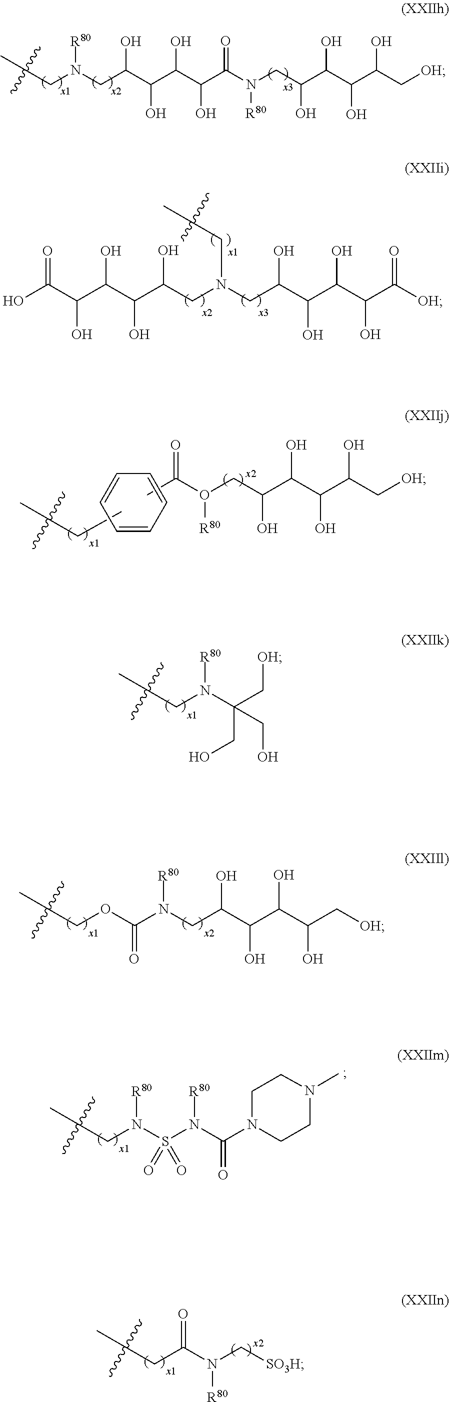

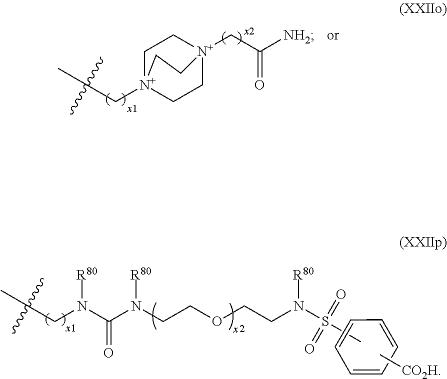

wherein:
R⁸⁰ is hydrogen or C₁₋₇alkyl;
R^{g} is independently, at each occurrence, hydrogen or C₁₋₇alkyl;
R^{h} is an electron pair, hydrogen or C₁₋₇alkyl; and
x1, x2 and x3 are each independently an integer ranging from 1 to 6.

In other embodiments of the foregoing, R⁸⁰ is hydrogen or methyl, and in other embodiments x1 is 2 or 3.

In even more embodiments, Q is -L²[(CH₂)ₘ₂O]ₘ₃(CH₂)ₘ₂R⁸⁶, wherein m2 is 2 or 3, m3 is an integer ranging from 1 to 21 and R⁸⁶ is hydrogen, hydroxyl or L³-I.

In even more embodiments, Q is -L²[(CH₂)ₘ₂O]ₘ₃(CH₂)ₘ₂R⁸⁶, wherein m2 is 2 or 3, m3 is an integer ranging from 1 to 21 and R⁸⁶ is hydrogen or hydroxyl.

In some other embodiments, Q has one of the following structures (XXIIIa), (XXIIIb) or (XXIIIc):

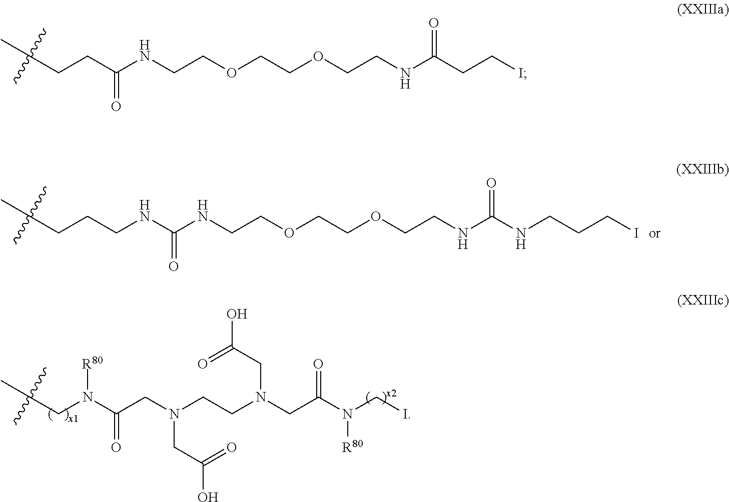

wherein I is a compound of structure (I).

In some embodiments, B has the following structure (XIV):

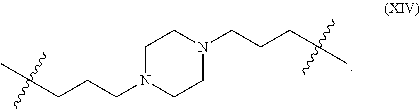 [SMILES: *CCCN1CCN(CCC*)CC1]

In certain embodiments, at least two of R⁸, R⁹, R¹⁰, R¹¹ or R¹² are selected from:

C₁₋₇-alkyl, C₂₋₇-alkenyl, C₂₋₇-alkinyl, halogen, halogen-C₁₋₇-alkyl, C₁₋₇-alkoxy, halogen-C₁₋₇-alkoxy, hydroxy, hydroxy-C₁₋₇-alkoxy, hydroxy-C₁₋₇-alkyl, hydroxy-C₃₋₇-alkenyl, hydroxy-C₃₋₇-alkynyl, cyano, carboxyl, C₁₋₇-alkoxycarbonyl, amino carbonyl, carboxyl-C₁₋₇-alkyl, carboxyl-C₂₋₇-alkenyl, carboxyl-C₂₋₇-alkynyl, C₁₋₇-alkoxycarbonyl-C₁₋₇-alkyl, C₁₋₇-alkoxycarbonyl-C₂₋₇-alkenyl, C₁₋₇-alkoxycarbonyl-C₂₋₇-alkynyl, carboxyl-C₁₋₇-alkoxy, C₁₋₇-alkoxycarbonyl-C₁₋₇-alkoxy, carboxyl-C₁₋₇-alkyl-aminocarbonyl, carboxyl-C₁₋₇-alkyl-(C₁₋₇-alkylamino)-carbonyl, C₁₋₇-alkoxycarbonyl-C₁₋₇-alkyl-aminocarbonyl, C₁₋₇-alkoxycarbonyl-C₁₋₇-alkyl-(C₁₋₇-alkylamino)-carbonyl, carboxyl-C₁₋₇-alkyl-aminocarbonyl-C₁₋₇-alkyl, carboxyl-C₁₋₇-alkyl-(C₁₋₇-alkylamino)-carbonyl-C₁₋₇-alkyl, C₁₋₇-alkoxycarbonyl-C₁₋₇-alkyl-aminocarbonyl-C₁₋₇-alkyl, C₁₋₇-alkoxycarbonyl-C₁₋₇-alkyl-(C₁₋₇-alkylamino)-carbonyl-C₁₋₇-alkyl, hydroxy-C₁₋₇-alkyl-aminocarbonyl, di-(hydroxy-C₁₋₇-alkyl)aminocarbonyl, aminocarbonyl-C₁₋₇-alkyl-amino carbonyl, hydroxysulfonyl-C₁₋₇-alkyl-aminocarbonyl, hydroxysulfonyl-C₁₋₇-alkyl-(C₁₋₇-alkyl-amino)-carbonyl, di-(C₁₋₇-alkoxycarbonyl-C₁₋₇-alkyl)-methylaminocarbonyl,

phenyl, wherein phenyl is unsubstituted or substituted by one to three groups selected from halogen, C₁₋₇-alkoxy, carboxyl and C₁₋₇-alkoxycarbonyl,

phenyl-carbonyl, wherein phenyl is unsubstituted or substituted by one to three groups selected from halogen, C₁₋₇-alkoxy, carboxyl and C₁₋₇-alkoxycarbonyl,

phenyl-aminocarbonyl, wherein phenyl is unsubstituted or substituted by one to three groups selected from halogen, C₁₋₇-alkoxy, carboxyl and C₁₋₇-alkoxycarbonyl,

phenyl-C₁₋₇-alkyl, wherein phenyl is unsubstituted or substituted by one to three groups selected from halogen, C₁₋₇-alkoxy, carboxyl and C₁₋₇-alkoxycarbonyl,

phenyl-C₂₋₇-alkynyl, wherein phenyl is unsubstituted or substituted by one to three groups selected from halogen, C₁₋₇-alkoxy, carboxyl and C₁₋₇-alkoxycarbonyl,

heteroaryl, wherein heteroaryl is unsubstituted or substituted by one to three groups selected from halogen, C₁₋₇-alkoxy, carboxyl and C₁₋₇-alkoxycarbonyl, heteroaryl-carbonyl, wherein heteroaryl is unsubstituted or substituted by one to three groups selected from halogen, C₁₋₇-alkoxy, carboxyl and C₁₋₇-alkoxycarbonyl,

heteroaryl-aminocarbonyl, wherein heteroaryl is unsubstituted or substituted by one to three groups selected from halogen, C₁₋₇-alkoxy, carboxyl and C₁₋₇-alkoxycarbonyl,

heteroaryl-C₁₋₇-alkyl, wherein heteroaryl is unsubstituted or substituted by one to three groups selected from halogen, C₁₋₇-alkyl, C₁₋₇-alkoxy, carboxyl and C₁₋₇-alkoxycarbonyl,

heteroaryl-C₁₋₇-alkyl-aminocarbonyl, wherein heteroaryl is unsubstituted or substituted by one to three groups selected from halogen, C-alkoxy, carboxyl and C₁₋₇-alkoxycarbonyl, and

heteroaryl-carbonyl-C₁₋₇-alkyl, wherein heteroaryl is unsubstituted or substituted by one to three groups selected from halogen, C₁₋₇-alkoxy, carboxyl and C₁₋₇-alkoxycarbonyl,

and the other ones of R⁸, R⁹, R¹⁰, R¹¹ and R¹² are hydrogen.

In even more embodiments, at least two of R⁸, R⁹, R¹⁰, R¹¹ and R¹² are selected from:

halogen, hydroxy, hydroxy-C₁₋₇-alkoxy, hydroxy-C₁₋₇-alkyl, cyano, carboxyl, C₁₋₇-alkoxycarbonyl, amino carbonyl, carboxyl-C₁₋₇-alkoxy, C₁₋₇-alkoxycarbonyl-C₁₋₇-alkoxy, carboxyl-C₁₋₇-alkyl-aminocarbonyl, carboxyl-C₁₋₇-alkyl-(C₁₋₇-alkylamino)-carbonyl, C₁₋₇-alkoxycarbonyl-C₁₋₇-alkyl-aminocarbonyl, hydroxy-C₁₋₇-alkyl-aminocarbonyl, di-(hydroxy-C₁₋₇-alkyl)aminocarbonyl, aminocarbonyl-C₁₋₇-alkyl-amino carbonyl, hydroxysulfonyl-C₁₋₇-alkyl-aminocarbonyl, hydroxysulfonyl-C₁₋₇-alkyl-(C₁₋₇-alkyl-amino)-carbonyl, di-(C₁₋₇-alkoxycarbonyl-C₁₋₇-alkyl)-methylaminocarbonyl,

phenyl-aminocarbonyl, wherein phenyl is unsubstituted or substituted by one to three groups selected from halogen, C₁₋₇-alkoxy, carboxyl and C₁₋₇-alkoxycarbonyl,

heteroaryl-aminocarbonyl, wherein heteroaryl is unsubstituted or substituted by one to three groups selected from halogen, C₁₋₇-alkoxy, carboxyl and C₁₋₇-alkoxycarbonyl,

heteroaryl-C₁₋₇-alkyl, wherein heteroaryl is unsubstituted or substituted by one to three groups selected from halogen, C₁₋₇-alkyl, C₁₋₇-alkoxy, carboxyl and C₁₋₇-alkoxycarbonyl,

heteroaryl-C₁₋₇-alkyl-aminocarbonyl, wherein heteroaryl is unsubstituted or substituted by one to three groups selected from halogen, C₁₋₇-alkoxy, carboxyl and C-alkoxycarbonyl, and

heteroaryl-carbonyl-C₁₋₇-alkyl, wherein heteroaryl is unsubstituted or substituted by one to three groups selected from halogen, C₁₋₇-alkoxy, carboxyl and C₁₋₇-alkoxycarbonyl,

and the other ones of R⁸, R⁹, R¹⁰, R¹¹ and R¹² are hydrogen.

In still other embodiments, at least one of R⁸, R⁹, R¹⁰, R¹¹ and R¹² is Q and at least one of R⁸, R⁹, R¹⁰, R¹¹ and R¹² are selected from:

C₁₋₇-alkyl, C₂₋₇-alkenyl, C₂₋₇-alkinyl, halogen, halogen-C₁₋₇-alkyl, C₁₋₇-alkoxy, halogen-C₁₋₇-alkoxy, hydroxy, hydroxy-C₁₋₇-alkoxy, hydroxy-C₁₋₇-alkyl, hydroxy-C₃₋₇-alkenyl, hydroxy-C₃₋₇-alkynyl, cyano, carboxyl, C₁₋₇-alkoxycarbonyl, amino carbonyl, carboxyl-C₁₋₇-alkyl, carboxyl-C₂₋₇-alkenyl, carboxyl-C₂₋₇-alkynyl, C₁₋₇-alkoxycarbonyl-C₁₋₇-alkyl, C₁₋₇-alkoxycarbonyl-C₂₋₇-alkenyl, C₁₋₇-alkoxycarbonyl-C₂₋₇-alkynyl, carboxyl-C₁₋₇-alkoxy, C₁₋₇-alkoxycarbonyl-C₁₋₇-alkoxy, carboxyl-C₁₋₇-alkyl-aminocarbonyl, carboxyl-C₁₋₇-alkyl-(C₁₋₇-alkylamino)-carbonyl, C₁₋₇-alkoxycarbonyl-C₁₋₇-alkyl-aminocarbonyl, C₁₋₇-alkoxycarbonyl-C₁₋₇-alkyl-(C₁₋₇-alkylamino)-carbonyl, carboxyl-C₁₋₇-alkyl-aminocarbonyl-C₁₋₇-alkyl, carboxyl-C₁₋₇-alkyl-(C₁₋₇-alkylamino)-carbonyl-C₁₋₇-alkyl, C₁₋₇-alkoxycarbonyl-C₁₋₇-alkyl-aminocarbonyl-C₁₋₇-alkyl, C₁₋₇-alkoxycarbonyl-C₁₋₇-alkyl-(C₁₋₇-alkylamino)-carbonyl-C₁₋₇-alkyl, hydroxy-C₁₋₇-alkyl-aminocarbonyl, di-(hydroxy-C₁₋₇-alkyl)aminocarbonyl, aminocarbonyl-C₁₋₇-alkyl-amino carbonyl, hydroxysulfonyl-C₁₋₇-alkyl-aminocarbonyl, hydroxysulfonyl-C₁₋₇-alkyl-(C₁₋₇-alkyl-amino)-carbonyl, di-(C₁₋₇-alkoxycarbonyl-C₁₋₇-alkyl)-methylaminocarbonyl,

phenyl, wherein phenyl is unsubstituted or substituted by one to three groups selected from halogen, C₁₋₇-alkoxy, carboxyl and C₁₋₇-alkoxycarbonyl,

phenyl-carbonyl, wherein phenyl is unsubstituted or substituted by one to three groups selected from halogen, C₁₋₇-alkoxy, carboxyl and C₁₋₇-alkoxycarbonyl,

phenyl-aminocarbonyl, wherein phenyl is unsubstituted or substituted by one to three groups selected from halogen, C₁₋₇-alkoxy, carboxyl and C₁₋₇-alkoxycarbonyl,

phenyl-C₁₋₇-alkyl, wherein phenyl is unsubstituted or substituted by one to three groups selected from halogen, C₁₋₇-alkoxy, carboxyl and C₁₋₇-alkoxycarbonyl,

phenyl-C₂₋₇-alkynyl, wherein phenyl is unsubstituted or substituted by one to three groups selected from halogen, C₁₋₇-alkoxy, carboxyl and C₁₋₇-alkoxycarbonyl,

heteroaryl, wherein heteroaryl is unsubstituted or substituted by one to three groups selected from halogen, C₁₋₇-alkoxy, carboxyl and C₁₋₇-alkoxycarbonyl,

heteroaryl-carbonyl, wherein heteroaryl is unsubstituted or substituted by one to three groups selected from halogen, C₁₋₇-alkoxy, carboxyl and C₁₋₇-alkoxycarbonyl,

heteroaryl-aminocarbonyl, wherein heteroaryl is unsubstituted or substituted by one to three groups selected from halogen, C₁₋₇-alkoxy, carboxyl and C₁₋₇-alkoxycarbonyl,

heteroaryl-C₁₋₇-alkyl, wherein heteroaryl is unsubstituted or substituted by one to three groups selected from halogen, C₁₋₇-alkyl, C₁₋₇-alkoxy, carboxyl and C₁₋₇-alkoxycarbonyl,

heteroaryl-C₁₋₇-alkyl-aminocarbonyl, wherein heteroaryl is unsubstituted or substituted by one to three groups selected from halogen, C-alkoxy, carboxyl and C₁₋₇-alkoxycarbonyl, and

heteroaryl-carbonyl-C₁₋₇-alkyl, wherein heteroaryl is unsubstituted or substituted by one to three groups selected from halogen, C₁₋₇-alkoxy, carboxyl and C₁₋₇-alkoxycarbonyl,

and the other ones of R⁸, R⁹, R¹⁰, R¹¹ and R¹² are hydrogen.

In still more embodiments, at least one of R⁸, R⁹, R¹⁰, R¹¹ and R¹² is Q and at least one of R⁸, R⁹, R¹⁰, R¹¹ and R¹² are selected from:

halogen, hydroxy, hydroxy-C₁₋₇-alkoxy, hydroxy-C₁₋₇-alkyl, cyano, carboxyl, C₁₋₇-alkoxycarbonyl, amino carbonyl, carboxyl-C₁₋₇-alkoxy, C₁₋₇-alkoxycarbonyl-C₁₋₇-alkoxy, carboxyl-C₁₋₇-alkyl-aminocarbonyl, carboxyl-C₁₋₇-alkyl-(C₁₋₇-alkylamino)-carbonyl, C₁₋₇-alkoxycarbonyl-C₁₋₇-alkyl-aminocarbonyl, hydroxy-C₁₋₇-alkyl-aminocarbonyl, di-(hydroxy-C₁₋₇-alkyl)aminocarbonyl, aminocarbonyl-C₁₋₇-alkyl-amino carbonyl, hydroxysulfonyl-C₁₋₇-alkyl-aminocarbonyl, hydroxysulfonyl-C₁₋₇-alkyl-(C₁₋₇-alkyl-amino)-carbonyl, di-(C₁₋₇-alkoxycarbonyl-C₁₋₇-alkyl)-methylaminocarbonyl,

phenyl-aminocarbonyl, wherein phenyl is unsubstituted or substituted by one to three groups selected from halogen, C₁₋₇-alkoxy, carboxyl and C₁₋₇-alkoxycarbonyl,

heteroaryl-aminocarbonyl, wherein heteroaryl is unsubstituted or substituted by one to three groups selected from halogen, C₁₋₇-alkoxy, carboxyl and C₁₋₇-alkoxycarbonyl,

heteroaryl-C₁₋₇-alkyl, wherein heteroaryl is unsubstituted or substituted by one to three groups selected from halogen, C₁₋₇-alkyl, C₁₋₇-alkoxy, carboxyl and C₁₋₇-alkoxycarbonyl,

heteroaryl-C₁₋₇-alkyl-aminocarbonyl, wherein heteroaryl is unsubstituted or substituted by one to three groups selected from halogen, C₁₋₇-alkoxy, carboxyl and C-alkoxycarbonyl, and

heteroaryl-carbonyl-C₁₋₇-alkyl, wherein heteroaryl is unsubstituted or substituted by one to three groups selected from halogen, C₁₋₇-alkoxy, carboxyl and C₁₋₇-alkoxycarbonyl,

and the other ones of R⁸, R⁹, R¹⁰, R¹¹ and R¹² are hydrogen.

In other embodiments, R⁸ and R¹¹ are halogen and R⁹, R¹⁰ and R¹² are hydrogen.

In certain embodiments, the compound is any one of Examples 1-291.

In other embodiments, the disclosure provides a compound which is a TGR5 agonist, wherein the TGR5 agonist stimulates GLP-1 secretion in a mammal and is active in the gastrointestinal tract of the mammal and wherein administration of the TGR5 agonist to the mammal does not induce filling of the gall bladder of the mammal as determined by ultrasound analysis.

In still other embodiments, the disclosure provides a compound which is TGR5 agonist, wherein the TGR5 agonist stimulates GLP-1 secretion in a mammal and is active in the gastrointestinal tract of the mammal and wherein administration of the TGR5 agonist to the mammal does not induce emptying of the gall bladder of the mammal as determined by ultrasound analysis.

In yet embodiments, the disclosure provides a compound which is TGR5 agonist, wherein the TGR5 agonist stimulates GLP-1 secretion in a mammal and is active in the gastrointestinal tract of the mammal and wherein administration of the TGR5 agonist to the mammal does not cause a change in weight of the mammal's gall bladder by more than 400% when compared to administration of a placebo. The change in weight of the mammal's gall bladder can be determined by any number of techniques known in the art. For example, in some embodiments change in weight of the mammal's gall bladder is determined in a mouse model.

In other embodiments of the forgoing, the TGR5 agonist administration does not cause a change in weight of the mammal's gall bladder by more than 300% when compared to administration of a placebo. In other embodiments of the forgoing, the TGR5 agonist administration does not cause a change in weight of the mammal's gall bladder by more than 200% when compared to administration of a placebo. In other embodiments of the forgoing, the TGR5 agonist administration does not cause a change in weight of the mammal's gall bladder by more than 100% when compared to administration of a placebo. In other embodiments of the forgoing, the TGR5 agonist administration does not cause a change in weight of the mammal's gall bladder by more than 50% when compared to administration of a placebo. In other embodiments of the forgoing, the TGR5 agonist administration does not cause a change in weight of the mammal's gall bladder by more than 10% when compared to administration of a placebo.

In some other embodiments, the disclosure provides a compound which is a TGR5 agonist, wherein the TGR5 agonist stimulates GLP-1 secretion in a mammal and is active in the gastrointestinal tract of the mammal and wherein the TGR5 agonist is administered to the mammal, the concentration of the TGR5 agonist in the gall bladder is less than about 100 μM. The amount of the TGR5 agonist in the mammal's gall bladder can be determined by any number of techniques known in the art. For example, in some embodiments the amount of the TGR5 agonist in the mammal's gall bladder is determined in a mouse model.

In still other embodiments, the TGR5 agonist concentration in the gall bladder is less than about 50 μM. In some other embodiments, the TGR5 agonist concentration in the gall bladder is less than about 25 μM. In other embodiments, the TGR5 agonist concentration in the gall bladder is less than about 10 μM. In still other embodiments, the TGR5 agonist concentration in the gall bladder is less than about 5 μM. In still other embodiments, the TGR5 agonist concentration in the gall bladder is less than about 1 μM. In still other embodiments, the TGR5 agonist concentration in the gall bladder is less than about 0.1 μM.

In some embodiments, the compounds have systemic exposure levels below their TGR5 EC50, yet they are still able to elicit a significant increase in plasma GLP-1 levels. For example, in some embodiments the disclosure provides a TGR5 agonist, wherein the TGR5 agonist stimulates GLP-1 secretion in a mammal and is active in the gastrointestinal tract of the mammal and wherein the TGR5 agonist is administered to the mammal, the concentration of the TGR5 agonist in the mammal's plasma is less than the TGR5 EC₅₀ of the TGR5 agonist. For example in some embodiments, the TGR5 agonist concentration in the mammal's plasma is less than about 50 ng/mL. In some other embodiments the TGR5 agonist concentration in the mammal's plasma is less than about 25 ng/mL. In some other embodiments the TGR5 agonist concentration in the mammal's plasma is less than about 10 ng/mL. In some other embodiments the TGR5 agonist concentration in the mammal's plasma is less than about 5 ng/mL. In yet other embodiments the TGR5 agonist concentration in the mammal's plasma is less than about 1 ng/mL.

In some other embodiments of any of the foregoing TGR5 agonists, the TGR5 agonist is not systemically available. In other embodiments of any of the foregoing TGR5 agonists, the TGR5 agonist concentration in the mammal's plasma is less than the TGR5 EC₅₀ of the TGR5 agonist. For example, in some embodiments the TGR5 agonist concentration in the mammal's plasma is less than about 50 ng/mL. In some other embodiments the TGR5 agonist concentration in the mammal's plasma is less than about 25 ng/mL. In some other embodiments the TGR5 agonist concentration in the mammal's plasma is less than about 10 ng/mL. In some other embodiments the TGR5 agonist concentration in the mammal's plasma is less than about 5 ng/mL. In yet other embodiments the TGR5 agonist concentration in the mammal's plasma is less than about 1 ng/mL.

In other embodiments of any of the forgoing TGR5 agonists, the TGR5 agonist does not modulate TGR5-mediated suppression of cytokines. In some other embodiments, the TGR5 agonist does not modulate the ileal bile acid transporter (IBAT). In yet other embodiments, the TGR5 agonist does not modulate the Farnesoid X Receptor (FXR).

In other embodiments of any of the foregoing TGR5 agonists, the TGR5 agonist stimulates PYY secretion.

In some embodiments of the foregoing, the TGR5 agonist is a compound of structure (I).

The compounds described herein are meant to include all racemic mixtures and all individual enantiomers or combinations thereof, whether or not they are specifically depicted herein. Further, the compounds are also intended to include all tautomeric forms, even if not specifically depicted. Tautomers are compounds which result from the formal migration of a hydrogen atom or proton, accompanied by a switch of a single bond and adjacent double bond.

Compounds as described herein may be in the free form or in the form of a salt thereof. In some embodiments, compounds as described herein may be in the form of a pharmaceutically acceptable salt, which are known in the art (Berge et al., J. Pharm. Sci. 1977, 66, 1). Pharmaceutically acceptable salt as used herein includes, for example, salts that have the desired pharmacological activity of the parent compound (salts which retain the biological effectiveness and/or properties of the parent compound and which are not biologically and/or otherwise undesirable). Compounds as described herein having one or more functional groups capable of forming a salt may be, for example, formed as a pharmaceutically acceptable salt. Compounds containing one or more basic functional groups may be capable of forming a pharmaceutically acceptable salt with, for example, a pharmaceutically acceptable organic or inorganic acid. Pharmaceutically acceptable salts may be derived from, for example, and without limitation, acetic acid, adipic acid, alginic acid, aspartic acid, ascorbic acid, benzoic acid, benzenesulfonic acid, butyric acid, cinnamic acid, citric acid, camphoric acid, camphorsulfonic acid, cyclopentanepropionic acid, diethylacetic acid, digluconic acid, dodecylsulfonic acid, ethanesulfonic acid, formic acid, fumaric acid, glucoheptanoic acid, gluconic acid, glycerophosphoric acid, glycolic acid, hemisulfonic acid, heptanoic acid, hexanoic acid, hydrochloric acid, hydrobromic acid, hydriodic acid, 2-hydroxyethanesulfonic acid, isonicotinic acid, lactic acid, malic acid, maleic acid, malonic acid, mandelic acid, methanesulfonic acid, 2-napthalenesulfonic acid, naphthalenedisulphonic acid, p-toluenesulfonic acid, nicotinic acid, nitric acid, oxalic acid, pamoic acid, pectinic acid, 3-phenylpropionic acid, phosphoric acid, picric acid, pimelic acid, pivalic acid, propionic acid, pyruvic acid, salicylic acid, succinic acid, sulfuric acid, sulfamic acid, tartaric acid, thiocyanic acid or undecanoic acid. Compounds containing one or more acidic functional groups may be capable of forming pharmaceutically acceptable salts with a pharmaceutically acceptable base, for example, and without limitation, inorganic bases based on alkaline metals or alkaline earth metals or organic bases such as primary amine compounds, secondary amine compounds, tertiary amine compounds, quaternary amine compounds, substituted amines, naturally occurring substituted amines, cyclic amines or basic ion-exchange resins. Pharmaceutically acceptable salts may be derived from, for example, and without limitation, a hydroxide, carbonate, or bicarbonate of a pharmaceutically acceptable metal cation such as ammonium, sodium, potassium, lithium, calcium, magnesium, iron, zinc, copper, manganese or aluminum, ammonia, benzathine, meglumine, methylamine, dimethylamine, trimethylamine, ethylamine, diethylamine, triethylamine, isopropylamine, tripropylamine, tributylamine, ethanolamine, diethanolamine, 2-dimethylaminoethanol, 2-diethylaminoethanol, dicyclohexylamine, lysine, arginine, histidine, caffeine, hydrabamine, choline, betaine, ethylenediamine, glucosamine, glucamine, methylglucamine, theobromine, purines, piperazine, piperidine, procaine, N-ethylpiperidine, theobromine, tetramethylammonium compounds, tetraethylammonium compounds, pyridine, N,N-dimethylaniline, N-methylpiperidine, morpholine, N-methylmorpholine, N-ethylmorpholine, dicyclohexylamine, dibenzylamine, N,N-dibenzylphenethylamine, 1-ephenamine, N,N′-dibenzylethylenediamine or polyamine resins. In some embodiments, compounds as described herein may contain both acidic and basic groups and may be in the form of inner salts or zwitterions, for example, and without limitation, betaines. Salts as described herein may be prepared by conventional processes known to a person skilled in the art, for example, and without limitation, by reacting the free form with an organic acid or inorganic acid or base, or by anion exchange or cation exchange from other salts. Those skilled in the art will appreciate that preparation of salts may occur in situ during isolation and purification of the compounds or preparation of salts may occur by separately reacting an isolated and purified compound.

Furthermore, all compounds of the invention which exist in free base or acid form can be converted to their pharmaceutically acceptable salts by treatment with the appropriate inorganic or organic base or acid by methods known to one skilled in the art. Salts of the compounds of the invention can be converted to their free base or acid form by standard techniques.

In some embodiments, compounds and all different forms thereof (e.g. free forms, salts, polymorphs, isomeric forms) as described herein may be in the solvent addition form, for example, solvates. Solvates contain either stoichiometric or non-stoichiometric amounts of a solvent in physical association the compound or salt thereof. The solvent may be, for example, and without limitation, a pharmaceutically acceptable solvent. For example, hydrates are formed when the solvent is water or alcoholates are formed when the solvent is an alcohol.

In some embodiments, compounds and all different forms thereof (e.g. free forms, salts, solvates, isomeric forms) as described herein may include crystalline and amorphous forms, for example, polymorphs, pseudopolymorphs, conformational polymorphs, amorphous forms, or a combination thereof. Polymorphs include different crystal packing arrangements of the same elemental composition of a compound. Polymorphs usually have different X-ray diffraction patterns, infrared spectra, melting points, density, hardness, crystal shape, optical and electrical properties, stability and/or solubility. Those skilled in the art will appreciate that various factors including recrystallization solvent, rate of crystallization and storage temperature may cause a single crystal form to dominate.

In some embodiments, compounds and all different forms thereof (e.g. free forms, salts, solvates, polymorphs) as described herein include isomers such as geometrical isomers, optical isomers based on asymmetric carbon, stereoisomers, tautomers, individual enantiomers, individual diastereomers, racemates, diastereomeric mixtures and combinations thereof, and are not limited by the description of the formula illustrated for the sake of convenience.

In some embodiments, pharmaceutical compositions in accordance with this invention may comprise a salt of such a compound, preferably a pharmaceutically or physiologically acceptable salt. Pharmaceutical preparations will typically comprise one or more carriers, excipients or diluents acceptable for the mode of administration of the preparation, be it by injection, inhalation, topical administration, lavage, or other modes suitable for the selected treatment. Suitable carriers, excipients or diluents are those known in the art for use in such modes of administration. Pharmaceutical compositions are described in more detail below.

It is understood that any embodiment of the compounds of structure (I), as set forth above, and any specific substituent set forth herein for R¹, R², R³, R⁴, R⁸, R⁹, R¹⁰, R¹¹, R¹², A¹, A², X, Y and Z group in the compounds of structure (I), as set forth above, may be independently combined with other embodiments and/or substituents of compounds of structure (I) to form embodiments of the inventions not specifically set forth above. In addition, in the event that a list of substitutents is listed for any particular R group in a particular embodiment and/or claim, it is understood that each individual substituent may be deleted from the particular embodiment and/or claim and that the remaining list of substituents will be considered to be within the scope of the invention. It is understood that in the present description, combinations of substituents and/or variables of the depicted formulae are permissible only if such contributions result in stable compounds.

The present disclosure also provides a pharmaceutical composition comprising any one or more of the compounds disclosed herein and a pharmaceutically acceptable carrier as described below.

### III. Preparation of Compounds

Compounds for use in the present invention may be obtained from commercial sources, prepared synthetically, obtained from naturally occurring sources or combinations thereof. Methods of preparing or synthesizing compounds of the present invention will be understood by a person of skill in the art having reference to known chemical synthesis principles.

The following Reaction Schemes I-IV illustrate methods for making compounds of this invention, i.e., compounds of structure (I):

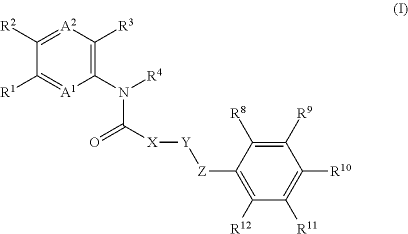

or a stereoisomer, tautomer, pharmaceutically acceptable salt or prodrug thereof, wherein R¹, R², R³, R⁴, R⁸, R⁹, R¹⁰, R¹¹, R¹², A¹, A², X, Y and Z are as defined above. It is understood that one skilled in the art may be able to make these compounds by similar methods or by combining other methods known to one skilled in the art. It is also understood that one skilled in the art would be able to make, in a similar manner as described below, other compounds of structure (I) not specifically illustrated below by using the appropriate starting components and modifying the parameters of the synthesis as needed. In general, starting components may be obtained from sources such as Sigma Aldrich, Lancaster Synthesis, Inc., Maybridge, Matrix Scientific, TCI, and Fluorochem USA, etc. or synthesized according to sources known to those skilled in the art (see, for example, Advanced Organic Chemistry: Reactions, Mechanisms, and Structure, 5th edition (Wiley, December 2000)) or prepared as described in this invention.

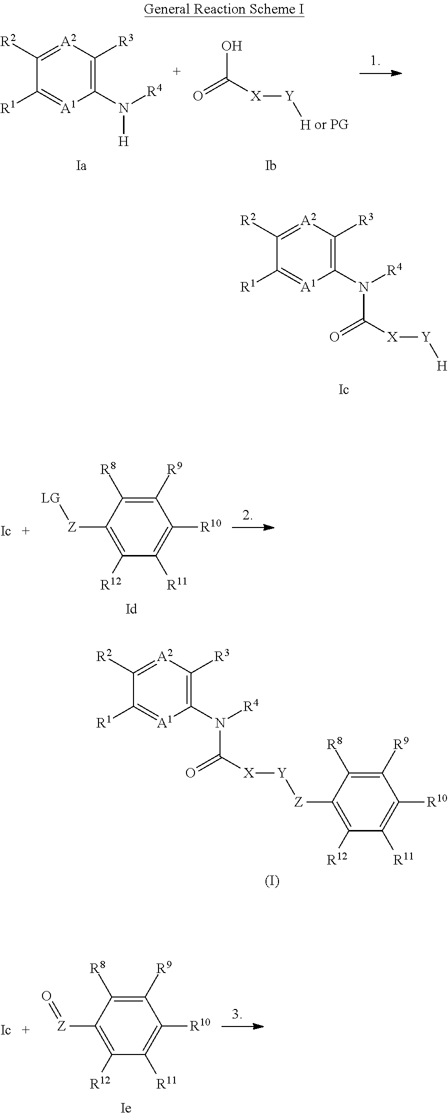

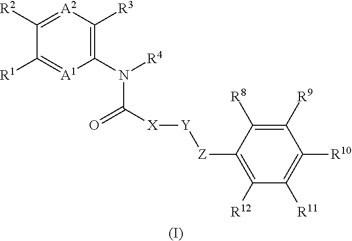

Referring to General Reaction Scheme I, an appropriate aromatic amine of structure Ia can be purchased or prepared according to methods known in the art and combined with an optional carboxyl activation reagent and/or acylation catalyst and a compound of structure Ib containing either a protected or free nucleophile (e.g., Y), to form compounds of structure Ic. Ic may then be reacted with either compounds of structure Id (LG is an appropriate leaving group) or Ie to form various compounds of structure (I). One skilled in the art will recognize that the methods may optionally include deprotection of PG and use of a hydride reducing agent where Ie comprises an arylaldehyde or arylketone.

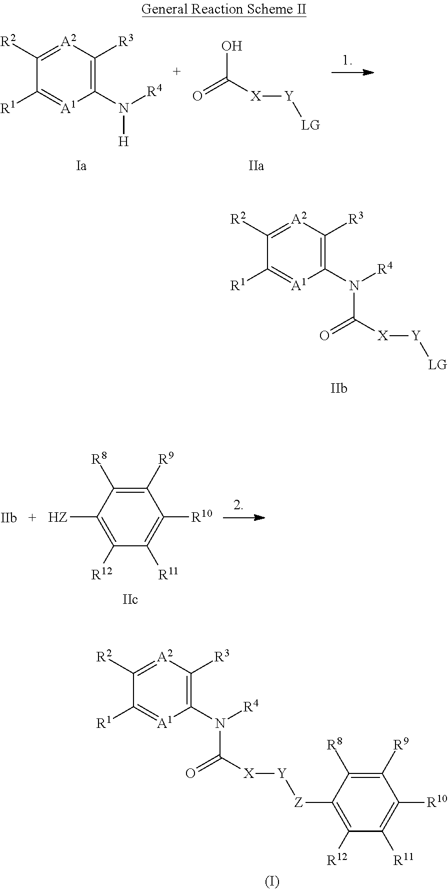

Alternatively, compounds of structure (I) may be prepared according to General Reaction Scheme II, wherein Ia is an appropriate aromatic amine and IIa is a carboxylate containing an electrophylic center Y, LG is a leaving group and Z is a nucleophile. Reaction of Ia with IIa may be performed in the presence of a carboxylate activation reagent, a base and an optional acylation catalyst. IIb can then be combined with a compound of structure IIc in the presence of an appropriate base to form various compounds of structure (I).

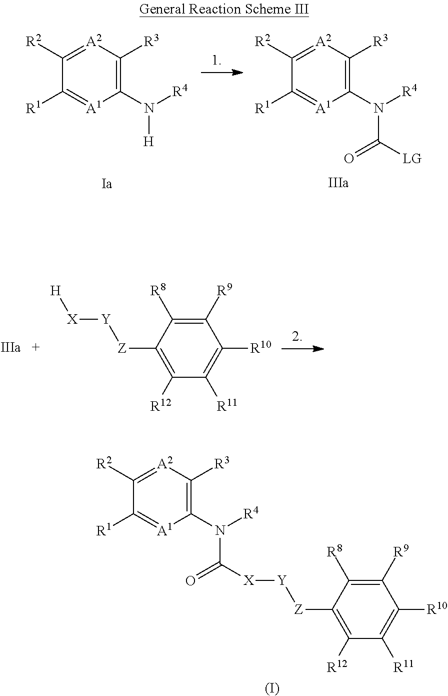

In another embodiment, compounds of structure (I) are prepared according to General Reaction Scheme III, wherein Ia is an appropriate aromatic amine, LG is a leaving group and X is a nucleophile. Reaction of Ia with a phosgene equivalent (LG-CO-LG, wherein LG is a leaving group) and an appropriate base results in compounds of structure IIIa. IIIa is then treated with an appropriate base, and an optional acylation catalyst to produce various compounds of structure (I).

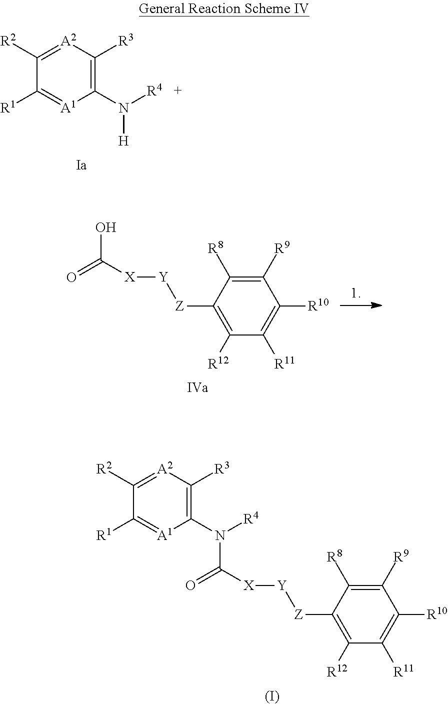

Other various compounds of structure (I) may be prepared according to General Reaction Scheme IV, wherein Ia is an appropriate aromatic amine and IIa is a carboxylate containing aryl group with appropriate linking elements X, Y, Z. Reaction of Ia and IIa in the presence of an appropriate carboxylate activation reagent, a base and an optional acylation catalyst results in various compounds of structure (I).

With regard to General Reaction Schemes I-IV, typical carboxylate activation reagents include DCC, EDCI, HATU, oxalyl chloride and the like. Typical bases include TEA, DIEA, pyridine, K₂CO₃, NaH and the like. Typical acylation catalysts include HOBt, HOAt, 4-dimethylaminopyridine and the like. Typical hydride reducing agents include NaBH₄, NaBH(OAc)₃, NaBH₃CN and the like. Typical phosgene equivalents include phosgene, triphosgene, carbonyldiimidazole, 4-nitrophenylchloroformate and the like.

One skilled in the art will recognize that variations to the order of the steps and reagents discussed in reference to General Reaction Scheme I are possible. Methodologies for preparation of compounds of structure (I) are described in more detail in the following non-limiting exemplary schemes.

It will also be appreciated by those skilled in the art that in the process described herein the functional groups of intermediate compounds may need to be protected by suitable protecting groups. Such functional groups include hydroxy, amino, mercapto and carboxylic acid. Suitable protecting groups for hydroxy include trialkylsilyl or diarylalkylsilyl (for example, t-butyldimethylsilyl, t-butyldiphenylsilyl or trimethylsilyl), tetrahydropyranyl, benzyl, and the like. Suitable protecting groups for amino, amidino and guanidino include t-butoxycarbonyl, benzyloxycarbonyl, and the like. Suitable protecting groups for mercapto include —C(O)—R″ (where R″ is alkyl, aryl or arylalkyl), p-methoxybenzyl, trityl and the like. Suitable protecting groups for carboxylic acid include alkyl, aryl or arylalkyl esters. Protecting groups may be added or removed in accordance with standard techniques, which are known to one skilled in the art and as described herein. The use of protecting groups is described in detail in Green, T. W. and P. G. M. Wutz, Protective Groups in Organic Synthesis (1999), 3rd Ed., Wiley. As one of skill in the art would appreciate, the protecting group may also be a polymer resin such as a Wang resin, Rink resin or a 2-chlorotrityl-chloride resin.

It will also be appreciated by those skilled in the art, although such protected derivatives of compounds of this invention may not possess pharmacological activity as such, they may be administered to a mammal and thereafter metabolized in the body to form compounds of the invention which are pharmacologically active. Such derivatives may therefore be described as “prodrugs”. All prodrugs of compounds of this invention are included within the scope of the invention.

By the methods described above, the representative compounds set forth in Examples 1-291 may be made, as well as by the more detailed procedures disclosed in the Examples.

### IV. TGR5 Methods

As mentioned above, new agents have recently been introduced to the market which prolong or mimic the effects of the naturally-secreted incretin hormones (Neumiller, J Am Pharm Assoc. 49(suppl 1):516-529, 2009). Another approach to initiating an incretin response involves the activation of TGR5, a bile acid sensitive G-protein coupled receptor (GPCR). TGR5 activation induces the secretion of incretins such as GLP-1 from the enteroendocrine L cells of the distal gut, thus providing the benefits of incretin therapy through an alternative mechanism. Activation of TGR5 might therefore be beneficial for the treatment of diabetes, obesity, metabolic syndrome, and related disorders. However, a key challenge remains in discovering how TGR5 agonism could generate a prolonged GLP-1 response, which would be necessary to achieve therapeutic benefit.

Bile acids (BA) are amphipathic molecules which are synthesized in the liver from cholesterol and stored in the gall bladder until secretion into the duodenum to play an important role in the solubilization and absorption of dietary fat and lipid-soluble vitamins. Approx. 99% of BA are absorbed again by passive diffusion and active transport in the terminal ileum and transported back to the liver via the portal vein (enterohepatic circulation). In the liver, BA decrease their own biosynthesis from cholesterol through the activation of the farnesoid X receptor alpha (FXRa) and small heterodimer partner (SHP), leading to the transcriptional repression of cholesterol 7α-hydroxylase, the rate-limiting step of BA biosynthesis from cholesterol. A G protein-coupled receptor responsive to bile acids, called TGR5, was independently identified by two investigators (Maruyama et al., “Identification of membrane-type receptor for bile acids (M-BAR)”Biochem. Biophys. Res. Comm. 298, 714-719, 2002; Kawamata et al., “A G Protein-coupled Receptor Responsive to Bile Acids” J. Biological Chem. 278, No. 11, 9435-9440, 2003), marking the first identification of cell surface receptors for this class of molecules. TGR5, in the literature also termed GPBAR1, M-BAR or BG37, is expressed in inflammation-mediating cells (e.g. macrophages), as well as a number of enteroendocrine derived cells lines such as GLUTtag, STC-1 and NCI-H716. Katsuma and colleagues demonstrated that bile acids could mediate the secretion of GLP-1 via TGR5 in STC-1 cells (Katsuma et al., Biochemical and Biophysical Research Communications 329:386-390, 2005).

TGR5 mRNA and protein have been reported to be expressed in a wide variety of tissues, although agreement on the sites of predominant expression appears to vary depending on the investigating group. It is clear that TGR5 mediates sensing of bile acids in, for example, brown fat, macrophages, gall bladder, and intestinal neurons; however, the function of this signaling is still being elucidated. While TGR5 has been found to be expressed in liver, it is not expressed in hepatocytes, but rather in liver sinusoidal endothelial cells and cholangiocytes (epithelial cells of the bile duct). This has implications for the role of TGR5 in bile acid regulation.

The compounds of the present invention are impermeable but still capable of inducing a TGR5-stimulated GLP-1 response, indicating that the TGR5 receptor may be present on the apical surface of the enteroendocrine L-cell in the GI tract. The development of methods to isolate primary L cells from mouse intestine (Reimann et al., Cell Metabolism 8:532-539, 2008) allowed confirmation that TGR5 was expressed in these GLP-1 secreting cells. In another study, a modestly active agonist of TGR5 was used to demonstrate a role for TGR5 in glucose homeostasis (Thomas et al., Cell Metabolism 10:167-177, 2009). In particular, they demonstrated that oral administration of INT-777 (EC50 of ˜1 μM vs. human TGR5; mouse potency not reported) to wild type mice resulted in an increase of plasma levels of GLP-1. When the experiment was performed in TGR5−/− mice, the response to NT-777 was not observed. Using NT-777 in a chronic diet-induced obesity model in mice, the investigators showed that the TGR5 agonist would improve glucose tolerance, an effect that was lost in the TGR5−/− mice. However, since this systemic TGR5 agonist also has significant effects on energy metabolism in mice due to its effects on brown fat and other tissues, it was unclear what contribution the enhanced GLP-1 expression had on the improvement of diet induced obesity.

TGR5 is also expressed in the gall bladder, and appears to modulate the filling and emptying of this organ. Vassileva and coworkers performed in situ hybridization experiments in TGR5 knockout mice and determined that there is significant TGR5 expression in the epithelial cells of the mouse gall bladder (Vassileva et al., Biochem. J. 398:423-430, 2006). They also demonstrated that TGR5 null mice are resistant to cholesterol gallstone disease when fed a lithogenic diet. In investigating the mechanism of resistance, they noted that the level of phospholipids was reduced in the total bile pool, indicating that the bile had a reduced cholesterol saturation index. They attributed this change to significantly higher hepatic expression levels of genes involved in bile acid synthesis (Cyp7a1 and Cyp27a1), and in hepatocellular uptake (Ntcp1 and Oatp1) in mice on the lithogenic diet, which suggests that the loss of TGR5 function impairs the negative feedback regulation of bile acid synthesis.

TGR5 protein is also expressed in human gallbladder epithelium (Keitel et al., Hepatology 50(3), 861-870, 2009). Keitel and coworkers examined 19 human gall bladder samples and detected TGR5 mRNA and protein in all samples tested. And although TGR5 mRNA was elevated in the presence of gallstones, no such relation was found for TGR5 protein levels. In addition, they found that TGR5 also localized in apical recycling endosomes, indicating that the receptor is regulated through translocation. The authors noted the significance of this finding, as in both cholangiocytes and gallbladder epithelium (which are exposed to millimolar bile acid concentrations) TGR5 is mainly localized in a subapical compartment and only to a smaller extent in the plasma membrane. In contrast, in sinusoidal endothelial cells and Kupffer cells (cells normally exposed to low bile acid concentrations) the receptor was predominantly detected within the plasma membrane.

It has also been reported that TGR5 mediated cAMP elevation can result in fluid and electrolyte secretion via activation and translocation of the cystic fibrosis transmembrane conductance regulator (CFTR). In addition to the presence of TGR5 in gallbladder epithelium, (Lavoie et al., J Physiol 588(17):3295-3305, 2010) demonstrated via PCR and immunohistochemistry that TGR5 is also expressed in gallbladder smooth muscle cells in the mouse. Functionally, they showed that bile acid TGR5 agonists could disrupt gallbladder smooth muscle function ex vivo, and that this disruption did not occur for tissues removed from TGR5−/− mice.

Additional functional confirmation of the role of TGR5 activation in gallbladder function came from the Mangelsdorf group, who used TGR5 knockout mice to demonstrate that TGR5 activation stimulates gallbladder filling (Li et al., Mol Endocrinol, 25(6), 1066-71, 2011). They demonstrated that i.p. injections of TGR5 agonists lithocholic acid (LCA) or NT-777 resulted in an approximately two-fold doubling of gallbladder volume in 30 minutes. The effect was completely blunted in the knockout mice. In further experiments examining direct effects on gallbladder smooth muscle in ex vivo tensiometry experiments, the investigators showed that both LCA and INT-777 markedly relaxed gallbladders from wild-type but not knockout mice, supporting the model that TGR5 acts directly on gallbladder to cause smooth muscle relaxation via induction of secondary messengers.

In aggregate, these studies indicate that TGR5 stimulation elicits gallbladder relaxation most likely via epithelial and/or smooth muscle TGR5 activation. The findings described above suggest that a TGR5 agonist being developed for diabetes should most preferably cause little or no activation of TGR5 in the biliary tree, as evidenced by lack of gallbladder filling during short or long term dosing.

In the small intestine, stimulation of TGR5 on enteroendocrine cells (L cells) by bile acids results in activation of adenylate cyclase (AC), thereby stimulating cAMP production and calcium influx. Increases in intracellular calcium and cAMP both lead to increased secretion of GLP-1 from L cells. Secreted GLP-1 has a number of effects. It augments glucose-dependent insulin release from β cells, it promotes β cell development, and it stimulates afferent nerves. GLP-1 also induces transcription of the insulin gene, thereby replenishing insulin stores. GLP-1 directly stimulates anorectic pathways in the hypothalamus and brain stem, resulting in a reduction in food intake.

While specific activation of TGR5 on the enteroendocrine cells of the GI tract offers distinct benefits to a diabetic population, activation of TGR5 receptors on tissues outside the GI tract, such as macrophages, liver sinusoidal endothelial cells (SECs), cholangiocytes (epithelial cells of the bile duct), and the like, can have unknown effects. For example, Kawamata and coworkers showed that bile acid treatment suppressed cytokine production in rabbit alveolar macrophages and TGR5-expressing monocytic cell line THP-1 (Kawamata, Journal of Biological Chemistry, 278(11):9435-9440, 2003) In macrophages, monocytes and Kupffer cells (liver resident macrophages) TGR5 activation inhibits cytokine release (interleukins (ILs) and tumor necrosis factor (TNF)-α). In liver SECs, TGR5 activation increases endothelial nitric oxide synthase (eNOS) activity, leading to nitric oxide production and vasodilation. Therefore, a preferred TGR5 agonist should ideally be capable of the bile-acid like stimulation of GI-resident L cells from the GI luminal side, but possess minimal to no systemic exposure and thereby avoid or minimize interactions with TGR5 receptors present on macrophages, cholangiocytes, tissues of the gall bladder, and the like. Although the compounds of the present invention are, in certain embodiments, impermeable, they are still capable of inducing a TGR5-stimulated GLP-1 response, indicating that the TGR5 receptor may be present on the apical surface of the enteroendocrine L-cell in the GI tract.

Accordingly, and in some embodiments, the present compounds find utility as TGR5 agonists and may be employed in methods for treating various conditions or diseases, including diabetes. Advantageously, some embodiments include compounds which are substantially non-systemically available. In certain embodiments, such compounds do not modulate filling or emptying of the gall bladder and in some embodiments may be present in the gall bladder in concentrations less than about 10 μM. While not wishing to be bound by theory, Applicants believe that certain functional groups on the compounds may contribute to the non-systemic availability of the compounds. For example, compounds of structure (I) which comprise polar functionality (e.g., a “Q” substituent having hydroxyl, guanidinyl, carboxyl, etc. substitutions) may be particularily useful as non-systemic TGR5 agonists.

In one embodiment the present disclosure provides the use of the disclosed compounds (compounds of structure (I)) as a therapeutically active substance, for example as a therapeutic active substance for the treatment of diseases which are associated with the modulation of TGR5 activity.

In other embodiments, the disclosure is directed to a method for the treatment of diseases which are associated with the modulation of TGR5 activity, wherein the diseases are selected from diabetes, Type II diabetes, gestational diabetes, impaired fasting glucose, impaired glucose tolerance, insulin resistance, hyperglycemia, obesity, metabolic syndrome, ischemia, myocardial infarction, retinopathy, vascular restenosis, hypercholesterolemia, hypertriglyceridemia, dyslipidemia or hyperlipidemia, lipid disorders such as low HDL cholesterol or high LDL cholesterol, high blood pressure, angina pectoris, coronary artery disease, atherosclerosis, cardiac hypertrophy, rheumatoid arthritis, asthma, chronic obstructive pulmonary disease (COPD), psoriasis, ulcerative colitis, Crohn's disease, disorders associated with parenteral nutrition especially during small bowel syndrome, irritable bowel syndrome (IBS), allergy diseases, fatty liver, non-alcoholic fatty liver disease (NAFLD), liver fibrosis, non-alcoholic steatohepatitis (NASH), primary sclerosing cholangitis (PSC), liver cirrhosis, primary biliary cirrhosis (PBC), kidney fibrosis, anorexia nervosa, bulimia nervosa and neurological disorders such as Alzheimer's disease, multiple sclerosis, schizophrenia and impaired cognition, the method comprising administering a therapeutically active amount of a compound of any one of claims 1-68 to a patient in need thereof.

In certain embodiments the disease is diabetes, and in other embodiments the disease is Type II diabetes or gestational diabetes.

The disclosure also provides use of the disclosed compounds (i.e., any compound of structure (I)) for the preparation of medicaments for the treatment of diseases which are associated with the modulation of TGR5 activity. For example, in certain embodiments the use is for the preparation of medicaments for the treatment a disease or condition selected from diabetes, Type II diabetes, gestational diabetes, impaired fasting glucose, impaired glucose tolerance, insulin resistance, hyperglycemia, obesity, metabolic syndrome, ischemia, myocardial infarction, retinopathy, vascular restenosis, hypercholesterolemia, hypertriglyceridemia, dyslipidemia or hyperlipidemia, lipid disorders such as low HDL cholesterol or high LDL cholesterol, high blood pressure, angina pectoris, coronary artery disease, atherosclerosis, cardiac hypertrophy, rheumatoid arthritis, asthma, chronic obstructive pulmonary disease (COPD), psoriasis, ulcerative colitis, Crohn's disease, disorders associated with parenteral nutrition especially during small bowl syndrome, irritable bowl disease (IBS), allergy diseases, fatty liver, liver fibrosis, liver cirrhosis, liver colestasis, primary biliary cirrhosis, primary scleroting cholangitis, kidney fibrosis, anorexia nervosa, bulimia nervosa and neurological disorders such as Alzheimer's disease, multiple sclerosis, schizophrenia and impaired cognition. In even other embodiments the disease is diabetes, and in other embodiments disease is Type II diabetes or gestational diabetes.

In still other embodiments, the disclosure provides a method for treating Type II diabetes mellitus in a patient in need thereof, the method comprising administering to the patient an effective amount of a compound of structure (I) or a pharmaceutical composition comprising the same.

In still other embodiments, the disclosure provides a method for treating inflammation of the GI tract in a patient in need thereof, the method comprising administering to the patient an effective amount of a compound of structure (I) or a pharmaceutical composition comprising the same. In certain embodiments, the use is for the preparation of medicaments for the treatment of a disease or condition selected from ulcerative colitis and Crohn's disease, conditions generally referred to in the aggregate as inflammatory bowel disease (IBD). In IBD, suppression of pro-inflammatory cytokine production within the GI tissues surrounding the lumen of the GI is a desirable attribute. Therefore, a preferred TGR5 agonist for the treatment of IBD should ideally be capable of the bile-acid like stimulation of GI-resident L cells from the GI luminal side as well as macrophages, monocytes and other cells resident in tissues surrounding the GI lumen but possess minimal to no systemic plasma exposure and thereby avoid or minimize interactions with TGR5 receptors present on cholangiocytes, tissues of the gall bladder, and the like.

In still other embodiments, the disclosure provides a method for stimulating GLP-1 secretion in a mammal, the method comprising administering a TGR5 agonist that is active in the gastrointestinal tract of the mammal and wherein the TGR5 agonist administration does not induce the filling of the gall bladder of the mammal as determined by ultrasound analysis.

In yet other embodiments, the disclosure provides a method for stimulating GLP-1 secretion in a mammal, the method comprising administering a TGR5 agonist that is active in the gastrointestinal tract of the patient and wherein the TGR5 agonist administration does not induce the emptying of the gall bladder of the mammal as determined by ultrasound analysis.

In some other embodiments, the disclosure provides a method for stimulating GLP-1 secretion in a mammal, the method comprising administering a TGR5 agonist that is active in the gastrointestinal tract of the patient and wherein the TGR5 agonist administration does not cause a change in weight of the mammal's gall bladder by more than 400% when compared to administration of a placebo. For example, in some embodiments the change in weight of the mammal's gall bladder is determined in a mouse model.

In certain embodiments of the forgoing, the TGR5 agonist administration does not cause a change in weight of the mammal's gall bladder by more than 300% when compared to administration of a placebo. In other embodiments, the TGR5 agonist administration does not cause a change in weight of the mammal's gall bladder by more than 200% when compared to administration of a placebo. In some other embodiments, the TGR5 agonist administration does not cause a change in weight of the mammal's gall bladder by more than 100% when compared to administration of a placebo. In other embodiments, the TGR5 agonist administration does not cause a change in weight of the mammal's gall bladder by more than 50% when compared to administration of a placebo. In certain other embodiments, the TGR5 agonist administration does not cause a change in weight of the mammal's gall bladder by more than 10% when compared to administration of a placebo.

Another embodiment is directed to a method for stimulating GLP-1 secretion in a mammal, the method comprising administering a TGR5 agonist that is active in the gastrointestinal tract of the mammal and wherein the TGR5 agonist concentration in the gall bladder is less than about 100 μM. The concentration of the TGR5 agonist in the gall bladder may be determined by any number of methods known in the art. For example, in some embodiments the TGR5 agonist concentration in the gall bladder is determined in a mouse model.

In other embodiments of the foregoing, the TGR5 agonist concentration in the gall bladder is less than about 50 μM, less than about 25 μM, less than about 10 μM, less than about 5 μM, less than about 1 μM or even less than about 0.1 μM.

In another embodiment, the present disclosure provides a method for stimulating GLP-1 secretion in a mammal, the method comprising administering a TGR5 agonist that is active in the gastrointestinal tract of the mammal and wherein the TGR5 agonist concentration in the mammal's plasma is less than the TGR5 EC₅₀ of the TGR5 agonist. For example, in some embodiments the TGR5 agonist concentration in the mammal's plasma is less than 50 ng/mL. In some other embodiments the TGR5 agonist concentration in the mammal's plasma is less than about 25 ng/mL. In some other embodiments the TGR5 agonist concentration in the mammal's plasma is less than about 10 ng/mL. In some other embodiments the TGR5 agonist concentration in the mammal's plasma is less than about 5 ng/mL. In yet other embodiments the TGR5 agonist concentration in the mammal's plasma is less than about 1 ng/mL.

In still other embodiments the disclosure provides a method for treating Type II diabetes mellitus in a patient in need thereof, the method comprising administering to the patient an effective amount of any of the disclosed TGR5 agonists or a pharmaceutical composition comprising the same. In some embodiments, the pharmaceutical composition comprises an additional therapeutic agent selected from the additional therapeutic agents described above.

In some other embodiments of any of the foregoing methods, the TGR5 agonist is not systemically available. In other embodiments of any of the foregoing TGR5 agonists, the TGR5 agonist concentration in the mammal's plasma is less than the TGR5 EC₅₀ of the TGR5 agonist. For example, in some embodiments the TGR5 agonist concentration in the mammal's plasma is less than about 50 ng/mL. In some other embodiments the TGR5 agonist concentration in the mammal's plasma is less than about 25 ng/mL. In some other embodiments the TGR5 agonist concentration in the mammal's plasma is less than about 10 ng/mL. In some other embodiments the TGR5 agonist concentration in the mammal's plasma is less than about 5 ng/mL. In yet other embodiments the TGR5 agonist concentration in the mammal's plasma is less than about 1 ng/mL.

In other embodiments of any of the forgoing methods, the TGR5 agonist does not modulate TGR5-mediated suppression of cytokines. In some other embodiments, the TGR5 agonist does not modulate the ileal bile acid transporter (IBAT). In yet other embodiments, the TGR5 agonist does not modulate the Farnesoid X Receptor (FXR).

In other embodiments of any of the foregoing methods, the TGR5 agonist stimulates PYY secretion. Enteroendocrine L-cells can be stimulated by nutrients and/or bile acids to co-secrete PYY and GLP-1. PYY plays an integral role in appetite control and energy homeostasis, and thus its co-release with GLP-1 in response to a TGR5 agonist could provide an added beneficial effect.

In other embodiments of any of the foregoing methods, the TGR5 agonist stimulates GLP-2 secretion. Enteroendocrine L-cells can be stimulated by nutrients and/or bile acids to co-secrete GLP-1 and GLP-2. GLP-2 plays an integral role in maintenance of the gastrointestinal mucosal epithelium and thus its co-release with GLP-1 in response to a TGR5 agonist could provide an added beneficial effect in conditions associated with disruption of the gastrointestinal mucosal epithelium. Pharmacological intervention with a GLP-2 agonist reduces the severity of damage in a rodent models of ulcerative colitis (Daniel J. Drucker et al., Am. J. Physiol. Gastrointest. Liver Physiol. 276, G79-G91, 1999 “Human [Gly2]GLP-2 Reduces the Severity of Colonic Injury in a Murine Model of Experimental Colitis” and Marie-Claude L'Heureux et al., J. Pharmacol. Exp. Ther. 306, 347-354, 2003 “Glucagon-Like Peptide-2 and Common Therapeutics in a Murine Model of Ulcerative Colitis”). For example, in certain embodiments the use is for the preparation of medicaments for the treatment a disease or condition selected from ulcerative colitis, Crohn's disease and disorders associated with parenteral nutrition especially during small bowel syndrome.

In certain other embodiments of any of the foregoing methods, the TGR5 agonist is a compound of structure (I).

In mammals such as mice, gallbladder phenotype (e.g. filled or empty) can be assessed surgically, by excising and weighing the gallbladder at a defined interval in an experiment. In humans and other higher mammals, there are also convenient and non-invasive ways to assess gallbladder phenotype. For example, Liddle and coworkers used abdominal ultrasonography to assess gallbladder volumes, wall thickening and the presence of gallstones or other pathology in human subjects taking a cholecystokinin (CCK) receptor antagonist (which blocks gallbladder emptying) (Liddle, J. Clin. Invest. 84:1220-1225, 1989). Such techniques can be used in the present invention to determine if a TGR5 agonist is affecting the filling or emptying of the gallbladder.

### V. Compositions and Administration

For the purposes of administration, the compounds of the present invention may be administered as a raw chemical or may be formulated as pharmaceutical compositions. Pharmaceutical compositions of the present invention comprise a compound of structure (I) and a pharmaceutically acceptable carrier, diluent or excipient. The compound of structure (I) is present in the composition in an amount which is effective to treat a particular disease or condition of interest—that is, in an amount sufficient to agonize TGR5, and preferably with acceptable toxicity to the patient. TGR5 activity of compounds of structure (I) can be determined by one skilled in the art, for example, as described in the Examples below. Appropriate concentrations and dosages can be readily determined by one skilled in the art.

Administration of the compounds of the invention, or their pharmaceutically acceptable salts, in pure form or in an appropriate pharmaceutical composition, can be carried out via any of the accepted modes of administration of agents for serving similar utilities. The pharmaceutical compositions of the invention can be prepared by combining a compound of the invention with an appropriate pharmaceutically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. Typical routes of administering such pharmaceutical compositions include, without limitation, oral, topical, transdermal, inhalation, parenteral, sublingual, buccal, rectal, vaginal, and intranasal. The term parenteral as used herein includes subcutaneous injections, intravenous, intramuscular, intrasternal injection or infusion techniques. Pharmaceutical compositions of the invention are formulated so as to allow the active ingredients contained therein to be bioavailable upon administration of the composition to a patient. Compositions that will be administered to a subject or patient take the form of one or more dosage units, where for example, a tablet may be a single dosage unit, and a container of a compound of the invention in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will, in any event, contain a therapeutically effective amount of a compound of the invention, or a pharmaceutically acceptable salt thereof, for treatment of a disease or condition of interest in accordance with the teachings of this invention.

In some embodiments, the disclosure provides a pharmaceutical composition comprising any of the foregoing compounds (i.e., a compound of structure (I)) and a pharmaceutically acceptable carrier or adjuvant.

In some embodiments, the disclosure provides a pharmaceutical composition comprising any of the foregoing compounds (i.e., a compound of structure (I)), a pharmaceutically acceptable carrier or adjuvant and one or more additional biologically active agents. For example, in some embodiments the one or more additional biologically active agents are selected from dipeptidyl peptidase 4 (DPP-4) inhibitors, biguanidines, sulfonylureas, α-glucosidates inhibitors, thiazolidinediones, incretin mimetics, CB1 antagonists, VPAC2 agonists, glucokinase activators, glucagon receptor antagonists, PEPCK inhibitors, SGLT1 inhibitors, SGLT2 inhibitors, IL-1 receptor antagonists, SIRT1 activators, SPPARMs and 11βHSD1 inhibitors.

In some other embodiments, the one or more additional biologically active agents prolong the TGR5-mediated GLP-1 signal. In other embodiments, the one or more additional biologically active agents are DPP-4 inhibitors. In still other embodiments, the one or more additional biologically active agents are sitagliptin, vildagliptin, saxagliptin, linagliptin, alogliptin, gemigliptin or dutogliptin. In even other embodiments, the one or more additional biologically active agents are selected from the group consisting of metformin or other biguanidine, glyburide or other sulfonyl urea, acarbose or other α-glucosidase inhibitor, rosiglitazone or other thiazolidinedione and exenatide or other incretin mimetic.

In some other embodiments, the present disclosure is directed to a pharmaceutical composition comprising any of TGR5 agonists described herein and a pharmaceutically acceptable carrier or adjuvant. For example, in some further embodiments of the foregoing, the pharmaceutical composition further comprises one or more additional biologically active agents. In some embodiments, the one or more additional biologically active agents are DPP-4 inhibitors. In other embodiments, the one or more additional biologically active agenta are sitagliptin, vildagliptin, saxagliptin, linagliptin, alogliptin, gemigliptin or dutogliptin.

A pharmaceutical composition of the invention may be in the form of a solid or liquid. In one aspect, the carrier(s) are particulate, so that the compositions are, for example, in tablet or powder form. The carrier(s) may be liquid, with the compositions being, for example, an oral syrup, injectable liquid or an aerosol, which is useful in, for example, inhalatory administration.

When intended for oral administration, the pharmaceutical composition is preferably in either solid or liquid form, where semi-solid, semi-liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like form. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent.

When the pharmaceutical composition is in the form of a capsule, for example, a gelatin capsule, it may contain, in addition to materials of the above type, a liquid carrier such as polyethylene glycol or oil.

The pharmaceutical composition may be in the form of a liquid, for example, an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid pharmaceutical compositions of the invention, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid pharmaceutical composition of the invention intended for either parenteral or oral administration should contain an amount of a compound of the invention such that a suitable dosage will be obtained.

The pharmaceutical composition of the invention may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device.

The pharmaceutical composition of the invention may be intended for rectal administration, in the form, for example, of a suppository, which will melt in the rectum and release the drug. The composition for rectal administration may contain an oleaginous base as a suitable nonirritating excipient. Such bases include, without limitation, lanolin, cocoa butter and polyethylene glycol.

The pharmaceutical composition of the invention may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule.

The pharmaceutical composition of the invention in solid or liquid form may include an agent that binds to the compound of the invention and thereby assists in the delivery of the compound. Suitable agents that may act in this capacity include a monoclonal or polyclonal antibody, a protein or a liposome.

The pharmaceutical composition of the invention may consist of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols of compounds of the invention may be delivered in single phase, bi-phasic, or tri-phasic systems in order to deliver the active ingredient(s). Delivery of the aerosol includes the necessary container, activators, valves, subcontainers, and the like, which together may form a kit. One skilled in the art, without undue experimentation may determine preferred aerosols.

The pharmaceutical compositions of the invention may be prepared by methodology well known in the pharmaceutical art. For example, a pharmaceutical composition intended to be administered by injection can be prepared by combining a compound of the invention with sterile, distilled water so as to form a solution. A surfactant may be added to facilitate the formation of a homogeneous solution or suspension. Surfactants are compounds that non-covalently interact with the compound of the invention so as to facilitate dissolution or homogeneous suspension of the compound in the aqueous delivery system.

The compounds of the invention, or their pharmaceutically acceptable salts, are administered in a therapeutically effective amount, which will vary depending upon a variety of factors including the activity of the specific compound employed; the metabolic stability and length of action of the compound; the age, body weight, general health, sex, and diet of the patient; the mode and time of administration; the rate of excretion; the drug combination; the severity of the particular disorder or condition; and the subject undergoing therapy.

Compounds of the invention, or pharmaceutically acceptable derivatives thereof, may also be administered simultaneously with, prior to, or after administration of one or more other therapeutic agents. For example, the compounds of the present invention may be administered with other therapeutically active compounds. Such methods are describe in more detail below. Such combination therapy includes administration of a single pharmaceutical dosage formulation which contains a compound of the invention and one or more additional active agents, as well as administration of the compound of the invention and each active agent in its own separate pharmaceutical dosage formulation. For example, a compound of the invention and the other active agent can be administered to the patient together in a single oral dosage composition such as a tablet or capsule, or each agent administered in separate oral dosage formulations. Where separate dosage formulations are used, the compounds of the invention and one or more additional active agents can be administered at essentially the same time, i.e., concurrently, or at separately staggered times, i.e., sequentially; combination therapy is understood to include all these regimens.

Suitable pharmaceutical compositions may be formulated by means known in the art and their mode of administration and dose determined by the skilled practitioner. For parenteral administration, a compound may be dissolved in sterile water or saline or a pharmaceutically acceptable vehicle used for administration of non-water soluble compounds such as those used for vitamin K. For enteral administration, the compound may be administered in a tablet, capsule or dissolved in liquid form. The tablet or capsule may be enteric coated, or in a formulation for sustained release. Many suitable formulations are known, including, polymeric or protein microparticles encapsulating a compound to be released, ointments, pastes, gels, hydrogels, or solutions which can be used topically or locally to administer a compound. A sustained release patch or implant may be employed to provide release over a prolonged period of time. Many techniques known to one of skill in the art are described in Remington: the Science & Practice of Pharmacy by Alfonso Gennaro, 20^{th} ed., Lippencott Williams & Wilkins, (2000). Formulations for parenteral administration may, for example, contain excipients, polyalkylene glycols such as polyethylene glycol, oils of vegetable origin, or hydrogenated naphthalenes. Biocompatible, biodegradable lactide polymer, lactide/glycolide copolymer, or polyoxyethylene-polyoxypropylene copolymers may be used to control the release of the compounds. Other potentially useful parenteral delivery systems for modulatory compounds include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

Compounds or pharmaceutical compositions in accordance with this invention or for use in this invention may be administered by means of a medical device or appliance such as an implant, graft, prosthesis, stent, etc. Also, implants may be devised which are intended to contain and release such compounds or compositions. An example would be an implant made of a polymeric material adapted to release the compound over a period of time.

It is to be noted that dosage values may vary with the severity of the condition to be alleviated. For any particular subject, specific dosage regimens may be adjusted over time according to the individual need and the professional judgement of the person administering or supervising the administration of the compositions. Dosage ranges set forth herein are exemplary only and do not limit the dosage ranges that may be selected by medical practitioners. The amount of active compound(s) in the composition may vary according to factors such as the disease state, age, sex, and weight of the subject. Dosage regimens may be adjusted to provide the optimum therapeutic response. For example, a single bolus may be administered, several divided doses may be administered over time or the dose may be proportionally reduced or increased as indicated by the exigencies of the therapeutic situation. It may be advantageous to formulate parenteral compositions in dosage unit form for ease of administration and uniformity of dosage.

In general, compounds of the invention should be used without causing substantial toxicity. Toxicity of the compounds of the invention can be determined using standard techniques, for example, by testing in cell cultures or experimental animals and determining the therapeutic index, i.e., the ratio between the LD50 (the dose lethal to 50% of the population) and the LD100 (the dose lethal to 100% of the population). In some circumstances however, such as in severe disease conditions, it may be necessary to administer substantial excesses of the compositions. Some compounds of this invention may be toxic at some concentrations. Titration studies may be used to determine toxic and non-toxic concentrations.

Compounds as described herein may be administered to a subject or patient. As used herein, a “subject” or “patient” may be a human, non-human primate, mammal, rat, mouse, cow, horse, pig, sheep, goat, dog, cat and the like.

Various alternative embodiments and examples of the invention are described herein. These embodiments and examples are illustrative and should not be construed as limiting the scope of the invention.

### EXAMPLES

### Example 1

### 1-(4-cyclopropyl-1,2,3,4-tetrahydroquinoxalin-1-yl)-2-[(2,5-dichlorophenyl)methoxy]-2-methylpropan-1-one

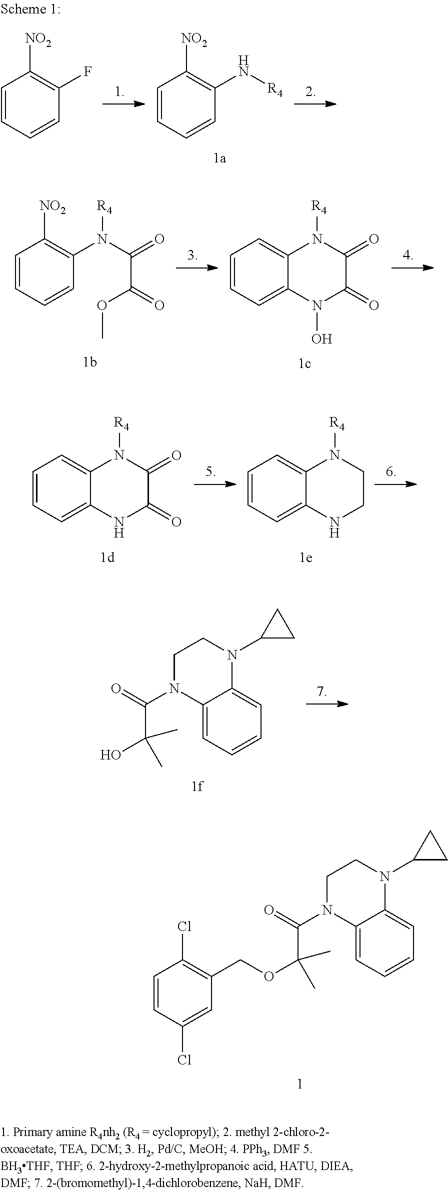

Intermediate 1a: N-cyclopropyl-2-nitroaniline. To cyclopropylamine (100 mL) was added 1-fluoro-2-nitrobenzene (30.0 g, 0.213 mol, 1.00 equiv) drop-wise with stirring. The reaction mixture was stirred overnight at 30° C. then diluted with water (100 mL), extracted with ethyl acetate (2×100 mL) and the organic layers combined. The combined organic extract was washed with brine (3×100 mL) dried over anhydrous sodium sulfate and concentrated under reduced pressure to provide 45 g (crude) N-cyclopropyl-2-nitroaniline as a yellow solid which was used without further purification.

Intermediate 1b: methyl [cyclopropyl(2-nitrophenyl)carbamoyl]formate. To a stirred 0° C. solution of N-cyclopropyl-2-nitroaniline (60 g, 0.337 mol, 1.00 equiv) and triethylamine (97.0 g, 0.959 mmol, 2.85 equiv) in dichloromethane (600 mL) was added methyl 2-chloro-2-oxoacetate (97.0 g, 0.792 mol, 2.35 equiv) drop-wise. The resulting reaction mixture was stirred for 3 h at 0-10° C. then diluted with of water (300 mL) and extracted with dichloromethane (600 mL). The organic phase was washed with of aqueous sodium carbonate (3×200 mL) and brine (2×200 mL), dried over anhydrous sodium sulfate and concentrated under reduced pressure to provide (88 g, 99%) of 1b as red oil. MS (ES, m/z): 265 [M+H]⁺.

Intermediate 1c: 1-cyclopropyl-4-hydroxy-1,2,3,4-tetrahydroquinoxaline-2,3-dione. Hydrogen gas was introduced into a stirred solution of [cyclopropyl(2-nitrophenyl)carbamoyl]formate (45.0 g, 0.170 mol, 1.00 equiv) and palladium on carbon (13 g) in methanol (400 mL). The resulting suspension was stirred for 3 h at 40° C. then solids were removed by filtration. The filter cake was washed with N,N-dimethylformamide, the combined filtrate was concentrated under reduced pressure to provide (31 g, 83%) of 1c as a white solid. MS (ES, m/z): 219 [M+H]⁺.

Intermediate 1d: 1-cyclopropyl-1,2,3,4-tetrahydroquinoxaline-2,3-dione. A stirred solution of 1-cyclopropyl-4-hydroxy-1,2,3,4-tetrahydroquinoxaline-2,3-dione (31.0 g, 0.142 mol, 1.00 equiv) and triphenylphosphine (56.0 g, 0.214 mol, 1.50 equiv) in N,N-dimethylformamide (250 mL) was purged and maintained under an atmosphere of nitrogen. The resulting solution was stirred for 2 h at 135° C. in an oil bath. The reaction mixture was cooled to 0° C. with an ice/water bath. Then diluted with of dichloromethane (300 mL) the solids were collected by filtration to provide (20 g, 70%) of 1d as a brown solid. MS (ES, m/z): 203 [M+H]⁺.

Intermediate 1e: 1-cyclopropyl-1,2,3,4-tetrahydroquinoxaline. To a solution of 1-cyclopropyl-1,2,3,4-tetrahydroquinoxaline-2,3-dione (20.0 g, 0.989 mol, 1.00 equiv) in tetrahydrofuran (100 mL) was added BH₃.THF (250 mL) the resulting solution was stirred for 4 h at 50° C. The reaction mixture was then quenched by addition of aqueous sodium carbonate (100 mL) then concentrated under reduced pressure, diluted with of water (200 mL) and extracted with ethyl acetate (2×200 mL). The combine organic extract was washed with brine (2×200 mL), dried over sodium sulfate and concentrated under reduced pressure to provide crude product residue. The residue was purified by silica gel column chromatography with and eluent gradient of petroleum ether:ethyl acetate (45:1 to 30:1) to furnish (11 g, 64%) of 1e as a white solid. MS (ES, m/z): 175 [M+H]⁺.

Intermediate 1f: 1-(4-cyclopropyl-1,2,3,4-tetrahydroquinoxalin-1-yl)-2-hydroxy-2-methylpropan-1-one. A solution of 1-cyclopropyl-1,2,3,4-tetrahydroquinoxaline (100 mg, 0.57 mmol, 1.0 equiv), 2-hydroxy-2-methylpropanoic acid (66 mg, 0.63 mmol, 1.10 equiv), HATU (262 mg, 0.69 mmol, 1.2 equiv) and DIEA (89 mg, 0.69 mmol, 1.2 equiv) in N,N-dimethylformamide (2 mL) was stirred overnight at room temperature. The resulting solution was diluted with of H₂O (5 mL) and extracted with of ethyl acetate (2×5 mL). The combined organic extract was washed with brine (1×10 mL), dried over anhydrous sodium sulfate and concentrated under reduced pressure. The residue was purified by preparative TLC (ethyl acetate:petroleum ether 1:5) to provide (30 mg, 20%) of 1f as yellow oil. MS (ES, m/z): 261 [M+H]⁺.

Example 1: 1-(4-cyclopropyl-1,2,3,4-tetrahydroquinoxalin-1-yl)-2-[(2,5-dichlorophenyl)methoxy]-2-methylpropan-1-one. To a solution of 1-(4-cyclopropyl-1,2,3,4-tetrahydroquinoxalin-1-yl)-2-hydroxy-2-methylpropan-1-one (30 mg, 0.12 mmol, 1.0 equiv) in N,N-dimethylformamide (2 mL) was added sodium hydride (15 mg, 0.62 mmol, 5.4 equiv) at 0° C. and the reaction mixture was stirred at this temperature for 15 min then 2-(bromomethyl)-1,4-dichlorobenzene (30 mg, 0.13 mmol, 1.1 equiv) was added. The reaction mixture was stirred overnight at room temperature then quenched by the addition of 5 mL of water. The resulting solution was extracted with ethyl acetate (2×5 mL) and the combined organic extract was washed with brine (1×10 mL), dried over anhydrous sodium sulfate and concentrated under reduced pressure. The residue was purified by preparative TLC with ethyl acetate:petroleum ether (1:1). The crude product (20 mg) was purified by preparative HPLC:Column, SunFire Prep-C18, 19*150 mm 5 μm; mobile phase gradient, water 0.05% TFA: CH₃CN (35% to 50% CH₃CN over 10 min; detector Waters 2545 UV detector 254/220 nm) to furnish (2.7 mg, 3%) of the title compound TFA salt as brown oil. MS (ES, m/z): 419 [M+H]⁺. ¹H-NMR (400 MHz, CD₃OD) δ 7.27-7.37 (m, 4H), 7.10-7.12 (m, 1H), 7.03 (t, J=7.6 Hz, 1H), 6.68 (t, J=7.6 Hz, 1H), 4.53 (s, 2H), 4.09 (s, 2H), 3.35-3.38 (m, 2H), 2.34-2.39 (m, 1H), 1.65 (s, 6H), 0.75-0.80 (m, 2H), 0.49 (m, 2H).

### Example 2

### (4-cyclopropyl-3,4-dihydroquinoxalin-1(2H)-yl)((2S,4R)-1-(2,5-dichlorobenzyl)-4-hydroxypyrrolidin-2-yl)methanone

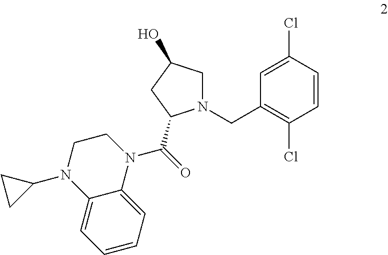 [SMILES: O=C([C@@H]1C[C@@H](O)CN1Cc1cc(Cl)ccc1Cl)N1CCN(C2CC2)c2ccccc21]

Example 2: (4-cyclopropyl-3,4-dihydroquinoxalin-1(2H)-yl)((2S,4R)-1-(2,5-dichlorobenzyl)-4-hydroxypyrrolidin-2-yl)methanone bis TFA salt. Example 2 was prepared using the procedures described in Example 6. MS (ES, m/z): 446 [M+H]⁺. ¹H-NMR (400 MHz, CD₃OD) δ 7.71 (s, 1H), 7.55 (s, 2H), 7.26 (s, 2H), 6.97 (d, J=8 Hz, 1H), 6.79 (m, 1H), 5.05 (t, J=8 Hz, 1H), 4.85-4.75 (m, 1H), 4.60 (m, 1H), 4.52 (m, 1H), 3.99 (m, 1H), 3.81 (m, 1H), 3.55 (m, 1H), 3.40-3.32 (m, 2H), 3.19-3.15 (m, 1H), 2.49 (s, 1H), 2.04-1.94 (m, 2H), 0.92-0.84 (m, 2H), 0.71-0.53 (m, 2H), 0.52 (d, J=8 Hz, 1H).

### Example 3

### (4-cyclopropyl-3,4-dihydroquinoxalin-1(2H)-yl)((2S,4S)-1-(2,5-dichlorobenzyl)-4-fluoropyrrolidin-2-yl)methanone

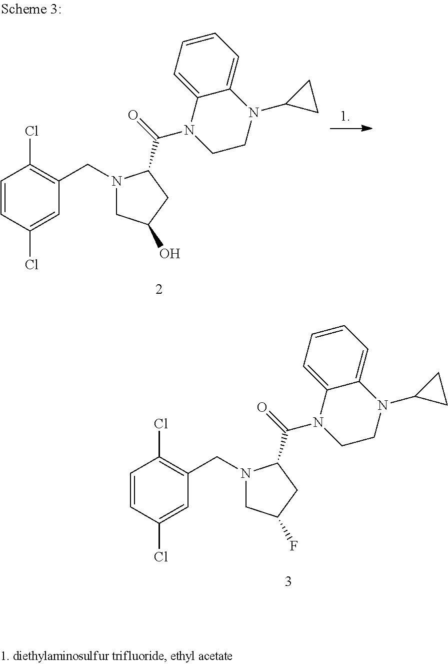 [SMILES: O=C([C@@H]1C[C@@H](O)CN1Cc1cc(Cl)ccc1Cl)N1CCN(C2CC2)c2ccccc21 ; O=C([C@@H]1C[C@H](F)CN1Cc1cc(Cl)ccc1Cl)N1CCN(C2CC2)c2ccccc21]

Example 3: (4-cyclopropyl-3,4-dihydroquinoxalin-1(2H)-yl)((2S,4S)-1-(2,5-dichlorobenzyl)-4-fluoropyrrolidin-2-yl)methanone. To Example 2 (40 mg, 0.090 mmol, 1.0 equiv) in ethyl acetate (6 mL) at 0° C. was added dropwise an ethyl acetate solution of diethylaminosulfur trifluoride (DAST; 36 mg, 0.22 mmol, 2.5 equiv) and the resulting solution was stirred overnight at room temperature. The mixture was diluted with 30 mL of ethyl acetate, washed with 1×20 mL of saturated aqueous sodium bicarbonate and 3×20 mL of brine, dried over sodium sulfate, concentrated and then purified by preparative reverse-phase HPLC to afford 40 mg (100%) of Example 3 bis TFA salt as a grey semi-solid. MS (ES, m/z): 448 [M+H]⁺. ¹H-NMR (400 MHz, CD₃OD) δ 7.81 (s, 1H), 7.49 (s, 2H), 7.27 (s, 2H), 7.05 (s, 1H), 6.81 (s, 1H), 5.44-5.31 (m, 1H), 5.05 (t, J=8 Hz, 1H), 4.57 (m, 1H), 3.99-3.83 (m, 2H), 3.83-3.62 (m, 2H), 3.46-3.40 (m, 1H), 3.27-3.23 (m, 1H), 2.48 (s, 2H), 2.30-2.13 (m, 2H), 0.89 (t, J=4 Hz, 2H), 0.66-0.54 (m, 2H).

### Example 4

### 1-(4-cyclopropyl-3,4-dihydroquinoxalin-1(2H)-yl)-2-(2,5-dichlorophenoxy)ethanone

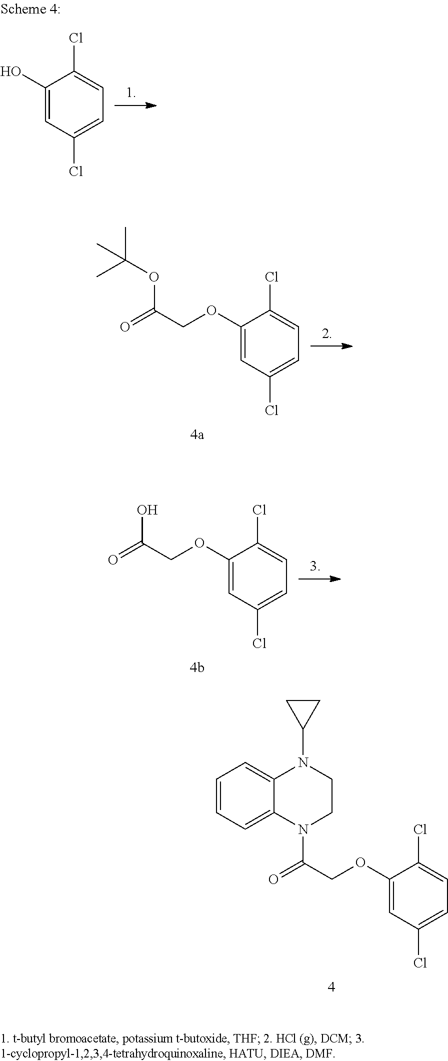 [SMILES: Oc1cc(Cl)ccc1Cl ; CC(C)(C)OC(=O)COc1cc(Cl)ccc1Cl ; O=C(O)COc1cc(Cl)ccc1Cl ; O=C(COc1cc(Cl)ccc1Cl)N1CCN(C2CC2)c2ccccc21]

Intermediate 4a: tert-butyl 2-(2,5-dichlorophenoxyl)acetate. To 2,5-dichlorophenol (300 mg, 1.84 mmol, 1.00 equiv) in THF (10 mL) was added potassium t-butoxide (400 mg, 3.56 mmol, 1.94 equiv) and the mixture was stirred for 20 min. To this was added t-butyl 2-bromoacetate (700 mg, 3.59 mmol, 1.95 equiv) and the reaction was stirred for 1 h at room temperature. The mixture was diluted with 10 mL of water, extracted with 2×20 mL of ethyl acetate, the organic layers combined and then washed with 2×15 mL of brine. The organic layer was dried, concentrated and then purified via silica gel chromatography, eluting with petroleum ether/ethyl acetate (30:1) to afford 300 mg (59%) of intermediate 4a as a colorless solid.

Intermediate 4b: 2-(2,5-dichlorophenoxy)acetic acid. To intermediate 4a (300 mg, 1.08 mmol, 1.00 equiv) in dichloromethane (10 mL) was bubbled hydrogen chloride gas and the solution then stirred for 5 h at 5° C. The reaction was concentrated to afford 350 mg (95%, purity ˜65%) of crude intermediate 4b as a white solid.

Example 4: 1-(4-cyclopropyl-3,4-dihydroquinoxalin-1(2H)-yl)-2-(2,5-dichlorophenoxy)ethanone. To 1-cyclopropyl-1,2,3,4-tetrahydroquinoxaline (200 mg, 1.15 mmol, 1.00 equiv) in DMF (10 mL) was added intermediate 4b (350 mg, 1.58 mmol, 1.40 equiv), HATU (655 mg, 1.72 mmol, 1.50 equiv) and DIEA (222 mg, 1.72 mmol, 1.50 equiv) and the resulting solution stirred for 2 h at 25° C. The reaction was diluted with 50 mL of water, extracted with 3×25 mL of ethyl acetate, the organic layers then combined, washed with 2×25 mL of brine and then dried over anhydrous sodium sulfate. The solution was concentrated and the residue purified via preparative reverse-phase HPLC to afford 76.9 mg of Example 4 TFA salt as a white solid. MS (ES, m/z): 377 [M+H]⁺. ¹H-NMR (400 MHz, DMSO-d₆) δ 7.45 (m, 2H), 7.01 (m, 4H), 6.66 (t, J=6.8 Hz, 1H), 5.13 (s, 2H), 3.74 (s, 2H), 3.36 (s, 2H), 2.45 (m, 1H), 0.84 (d, J=6 Hz, 2H), 0.58 (s, 2H).

### Example 5

### (S)-(4-cyclopropyl-3,4-dihydroquinoxalin-1(2H)-yl)(1-(2,5-dichlorobenzyl)-4,4-difluoropyrrolidin-2-yl)methanone

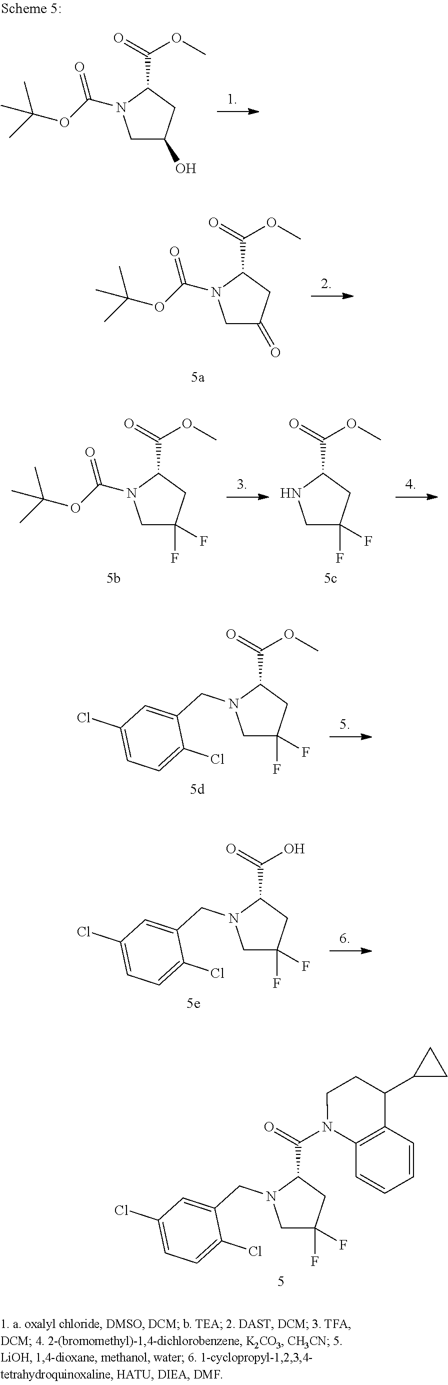

Intermediate 5a: (S)-1-tert-butyl 2-methyl 4-oxopyrrolidine-1,2-dicarboxylate. To a solution of DMSO (1.90 g, 24.3 mmol, 3.00 equiv) in dichloromethane (20 mL) at −78° C. was added oxalyl chloride (1.54 g, 12.1 mmol, 1.50 equiv) and the mixture was stirred for 15 min. To this was added dropwise a solution of 1-tert-butyl 2-methyl(2S,4R)-4-hydroxypyrrolidine-1,2-dicarboxylate (2.00 g, 8.15 mmol, 1.00 equiv) in dichloromethane (8 mL) and the mixture was stirred for 60 min at −78˜−60° C. The solution was allowed to warm to RT and triethylamine (4.90 g, 48.4 mmol, 6.00 equiv) was added. The mixture was then diluted with 50 mL of dichloromethane, washed with 2×30 mL of brine, dried over anhydrous sodium sulfate, concentrated under reduced pressure and then purified via silica gel chromatography (dichloromethane/methanol, 10:1) to afford 1 g (50%) of intermediate 5a as a yellow oil.

Intermediate 5b: (S)-1-tert-butyl 2-methyl 4,4-difluoropyrrolidine-1,2-dicarboxylate. To intermediate 5a (300 mg, 1.23 mmol, 1.00 equiv) in dichloromethane (30 mL) at 0° C. was added dropwise a solution of DAST (1.80 g, 11.2 mmol, 9.00 equiv) in dichloromethane (10 mL) and the resulting solution was stirred overnight at room temperature. The mixture was then washed with 1×30 mL of saturated aqueous sodium bicarbonate and 3×30 mL of brine, the organic layer was dried over anhydrous sodium sulfate and then concentrated under reduced pressure to afford 300 mg (92%) of intermediate 5b as yellow oil.

Intermediate 5c: (S)-methyl 4,4-difluoropyrrolidine-2-carboxylate. To intermediate 5b (300 mg, 1.13 mmol, 1.00 equiv) in dichloromethane (1 mL) was added trifluoroacetic acid (1 mL) and the resulting solution was stirred for 1 h at room temperature. The mixture was then concentrated under reduced pressure to afford 200 mg (crude) of intermediate 5c as a brown oil.

Intermediate 5d: (S)-methyl 1-(2,5-dichlorobenzyl)-4,4-difluoropyrrolidine-2-carboxylate. To intermediate 5c (200 mg, 1.21 mmol, 1.00 equiv) in CH₃CN (5 mL) was added 2-(bromomethyl)-1,4-dichlorobenzene (288 mg, 1.20 mmol, 1.00 equiv) and potassium carbonate (502 mg, 3.63 mmol, 3.00 equiv) and the resulting solution was stirred overnight at room temperature. The mixture was diluted with 50 mL of ethyl acetate, washed with 2×30 mL of brine, the organic layer dried over anhydrous sodium sulfate, concentrated, and then purified via silica gel chromatography (petroleum ether/ethyl acetate, 50:1) to afford 200 mg (51%) of intermediate 5d as a yellow oil.

Intermediate 5e: (S)-1-(2,5-dichlorobenzyl)-4,4-difluoropyrrolidine-2-carboxylic acid. To intermediate 5d (170 mg, 0.52 mmol, 1.0 equiv) in 1,4-dioxane/CH₃OH/H₂O (3:2:1 mL) was added LiOH.H₂O (44.0 mg, 1.05 mmol, 2.00 equiv) and the resulting solution was stirred for 60 min at 80° C. The pH value of the solution was adjusted to 6 with aqueous 2M HCl and the resulting mixture concentrated under reduced pressure to afford 120 mg (74%) of intermediate 5e as yellow oil.

Example 5: (S)-(4-cyclopropyl-3,4-dihydroquinoxalin-1(2H)-yl)(1-(2,5-dichlorobenzyl)-4,4-difluoropyrrolidin-2-yl)methanone. To intermediate 5e (120 mg, 0.39 mmol, 1.0 equiv) in DMF (5 mL) was added 1-cyclopropyl-1,2,3,4-tetrahydroquinoxaline (67.2 mg, 0.39 mmol, 1.00 equiv), HATU (294 mg, 0.77 mmol, 2.0 equiv) and DIEA (96.6 mg, 0.75 mmol, 2.0 equiv) and the resulting solution stirred overnight at room temperature. The mixture was diluted with 30 mL of ethyl acetate, washed with 3×20 mL of brine, and the organic layer dried over sodium sulfate. The crude product was purified by preparative reverse-phase HPLC to afford 20 mg (11%) of Example 5 bis TFA salt as a white solid.

### Example 6

### (4-cyclopropyl-3,4-dihydroquinoxalin-1(2H)-yl)((2S,4S)-1-(2,5-dichlorobenzyl)-4-hydroxypyrrolidin-2-yl)methanone

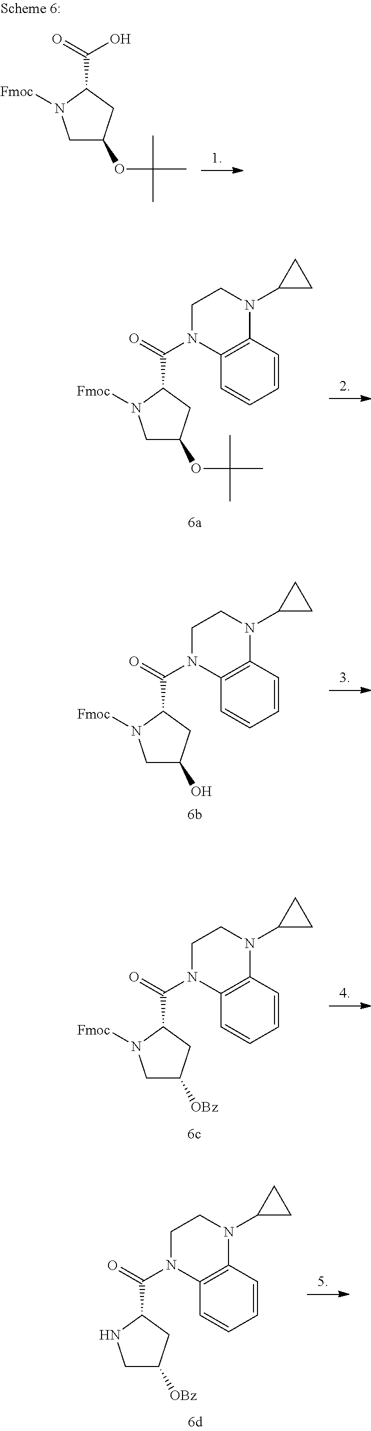

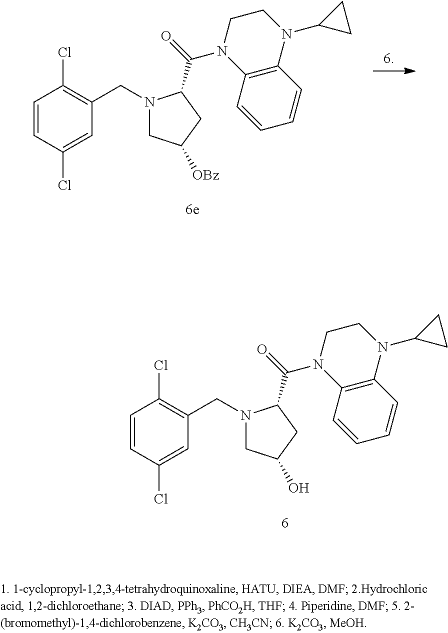

Intermediate 6a: (2S,4R)-(9H-fluoren-9-yl)methyl 4-tert-butoxy-2-(4-cyclopropyl-1,2,3,4-tetrahydroquinoxaline-1-carbonyl)pyrrolidine-1-carboxylate. To 1-cyclopropyl-1,2,3,4-tetrahydroquinoxaline (400 mg, 2.30 mmol, 1.00 equiv) in N,N-dimethylformamide (8 mL) was added (2S,4R)-4-(tert-butoxy)-1-[(9H-fluoren-9-ylmethoxy)carbonyl]pyrrolidine-2-carboxylic acid (940 mg, 2.30 mmol, 1.00 equiv), HATU (1.30 g, 3.42 mmol, 1.50 equiv) and DIEA (444 mg, 3.44 mmol, 1.50 equiv) and the mixture was stirred overnight at room temperature. The resulting solution was diluted with 40 mL of ethyl acetate, washed with 4×30 mL of brine, dried over sodium sulfate, filtered and then concentrated under reduced pressure to afford 1.5 g (crude) of intermediate 6a as a blue solid.

Intermediate 6b: (2S,4R)-(9H-fluoren-9-yl)methyl 2-(4-cyclopropyl-1,2,3,4-tetrahydroquinoxaline-1-carbonyl)-4-hydroxypyrrolidine-1-carboxylate. To intermediate 6a (300 mg, 0.53 mmol, 1.00 equiv) in 1,2-dichloroethane (10 mL) was added concentrated hydrochloric acid (1 mL) and the resulting solution was stirred overnight at room temperature. The pH value of the solution was adjusted to 9 with saturated aqueous sodium carbonate then extracted with 3×20 mL of dichloromethane. The organic layers were combined, washed with 3×20 mL of brine, dried over sodium sulfate, filtered and concentrated under reduced pressure to afford 180 mg (67%) of intermediate 6b as a blue oil.

Intermediate 6c: (2S,4S)-(9H-fluoren-9-yl)methyl 4-(benzoyloxy)-2-(4-cyclopropyl-1,2,3,4-tetrahydroquinoxaline-1-carbonyl)pyrrolidine-1-carboxylate. To intermediate 1b (120 mg, 0.24 mmol, 1.0 equiv) in tetrahydrofuran (8 mL) at 0° C. was added PPh₃ (144 mg, 0.55 mmol, 2.4 equiv) and benzoic acid (72 mg, 0.59 mmol, 2.4 equiv) followed by the dropwise addition of a solution of DIAD (120 mg, 0.59 mmol, 2.4 equiv) in tetrahydrofuran (1 mL). The resulting solution was stirred for 2 h at room temperature then diluted with 40 mL of ethyl acetate, washed with 2×30 mL of brine and then dried over anhydrous sodium sulfate. The mixture was concentrated then applied onto a silica gel column, eluting with petroleum ether/ethyl acetate (5:1) to afford 200 mg (crude) of intermediate 6c as yellow oil.

Intermediate 6d: (3S,5S)-5-(4-cyclopropyl-1,2,3,4-tetrahydroquinoxaline-1-carbonyl)pyrrolidin-3-yl benzoate. To intermediate 1c (200 mg, 0.33 mmol, 1.00 equiv) in DMF (5 mL) was added piperidine (1 mL) and the resulting solution was stirred overnight at room temperature. The mixture was diluted with 30 mL of ethyl acetate, washed with 4×20 mL of brine, dried over sodium sulfate, filtered and then concentrated under reduced pressure to afford 150 mg (crude) intermediate 6d.

Intermediate 6e: (3S,5S)-5-(4-cyclopropyl-1,2,3,4-tetrahydroquinoxaline-1-carbonyl)-1-(2,5-dichlorobenzyl)pyrrolidin-3-yl benzoate. To intermediate 1c (300 mg, 0.77 mmol, 1.0 equiv) in CH₃CN (10 mL) was added 2-(bromomethyl)-1,4-dichlorobenzene (180 mg, 0.75 mmol, 1.0 equiv) and potassium carbonate (300 mg, 2.15 mmol, 3.00 equiv) and the resulting suspension was stirred for 2 h at room temperature. The solids were filtered out and the filtrate was concentrated under reduced pressure to afford 300 mg (71%) of intermediate 6e as a yellow oil.

Example 6: (4-cyclopropyl-3,4-dihydroquinoxalin-1(2H)-yl)((2S,4S)-1-(2,5-dichlorobenzyl)-4-hydroxypyrrolidin-2-yl)methanone. To intermediate 1e (300 mg, 0.54 mmol, 1.0 equiv) in methanol (8 mL) was added potassium carbonate (226 mg, 1.64 mmol, 3.00 equiv.) and the resulting solution was stirred for 60 min at room temperature. The mixture was concentrated, the residue was dissolved in 30 mL of ethyl acetate, washed with 3×20 mL of brine, dried over anhydrous sodium sulfate, then filtered and concentrated under reduced pressure. The crude product (200 mg) was purified by preparative reverse-phase HPLC to afford 30.7 mg of the title compound bis TFA salt as a light yellow solid. MS (ES, m/z): 446 [M+H]⁺. ¹H-NMR (400 MHz, CD₃OD) δ 7.81 (s, 1H), 7.55 (d, J=8 Hz, 2H), 7.27 (m, 2H), 7.07 (d, J=8 Hz, 1H), 6.79 (m, 1H), 4.97 (t, J=8 Hz, 1H), 4.67-4.58 (m, 2H), 4.42 (m, 1H), 4.14 (m, 1H), 3.80-3.72 (m, 1H), 3.63 (m, 1H), 3.49-3.43 (m, 3H), 3.18 (m, 1H), 2.51 (t, J=4 Hz, 1H), 2.38 (m, 1H), 1.72 (d, J=8 Hz, 1H), 0.93-0.87 (m, 2H), 0.75-0.65 (m, 1H), 0.53 (t, J=4 Hz, 1H).

### Example 7

### 1-(4-cyclopropyl-3,4-dihydroquinoxalin-1(2H)-yl)-2-(2,5-dichlorobenzylamino)propan-1-one

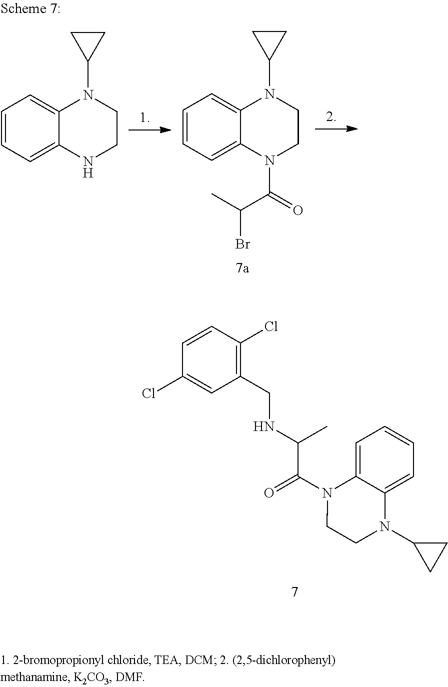 [SMILES: c1ccc2c(c1)NCCN2C1CC1 ; CC(Br)C(=O)N1CCN(C2CC2)c2ccccc21 ; CC(NCc1cc(Cl)ccc1Cl)C(=O)N1CCN(C2CC2)c2ccccc21]

Intermediate 7a: 2-bromo-1-(4-cyclopropyl-3,4-dihydroquinoxalin-1(2H)-yl)propan-1-one. To a solution of 1-cyclopropyl-1,2,3,4-tetrahydroquinoxaline (600 mg, 3.44 mmol, 1.00 equiv) in DCM (20 mL) at 0° C. was added triethylamine (697 mg, 6.89 mmol, 2.00 equiv) followed by the dropwise addition of 2-bromopropanoyl chloride (1.17 g, 6.84 mmol, 2.00 equiv) and the resulting solution was allowed to warm to room temperature and then stirred for 3 h. The mixture was diluted with dichloromethane (50 mL), washed with 2×50 mL of brine, dried over anhydrous sodium sulfate and then concentrated to afford 690 mg (65%) of intermediate 7a as a yellow oil.

Example 7: 1-(4-cyclopropyl-3,4-dihydroquinoxalin-1(2H)-yl)-2-(2,5-dichlorobenzylamino)propan-1-one. To intermediate 7a (600 mg, 1.94 mmol, 1.00 equiv) in DMF (10 mL) was added (2,5-dichlorophenyl)methanamine (341 mg, 1.94 mmol, 1.00 equiv) and potassium carbonate (542 mg, 3.92 mmol, 2.00 equiv) and the reaction was stirred for 3 h at 60° C. The reaction was diluted with 50 mL of ethyl acetate, washed with water (2×50 mL), brine (2×50 mL), dried over anhydrous sodium sulfate and then concentrated. The residue was purified by preparative TLC (petroleum ether/ethyl acetate (4:1)) followed by preparative reverse-phase HPLC to afford 30.2 mg (4%) of Example 7 bis TFA salt as a pink oil. MS (ES, m/z): 404 [M+H]⁺. ¹H-NMR (300 MHz, CD₃OD) δ 7.70 (s, 1H), 7.56-7.47 (m, 2H), 7.29-7.22 (m, 2H), 7.14-7.12 (m, 1H), 7.56-7.47 (m, 2H), 6.81-6.70 (m, 1H), 4.73-4.59 (m, 1H), 4.46-4.30 (m, 3H), 3.83 (s, 1H), 3.54-3.31 (m, 3H), 2.51 (brs, 1H), 1.63 (s, 1H), 1.24 (d, J=6.9 Hz, 2H), 0.96-0.82 (m, 2H), 0.73-0.62 (m, 1H), 0.52-0.48 (m, 3H).

### Example 8

### (4-cyclopropyl-3,4-dihydroquinoxalin-1(2H)-yl)(1-(2,5 dichlorobenzyloxy)cyclopentyl)methanone

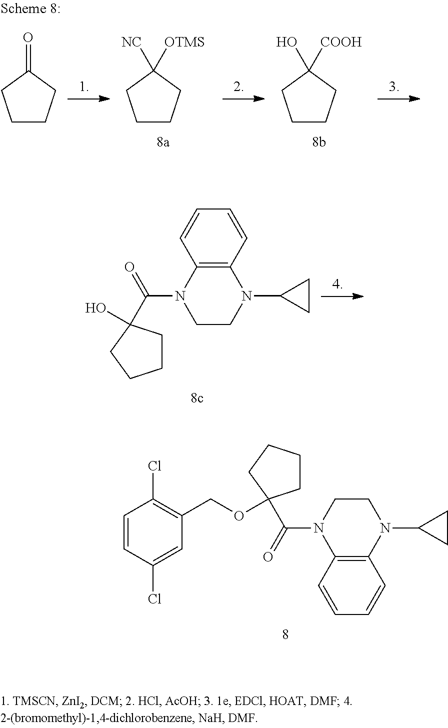

Intermediate 8a 1-(trimethylsilyloxy)cyclopentanecarbonitrile: Cyclopentanone (2 g, 23.78 mmol), TMSCN (3.53 g, 35.66 mmol), and ZnI₂ (890 mg, 2.79 mmol) were dissolved in dichloromethane (20 mL). The resulting solution was stirred for 6 h at room temperature, then diluted with 20 mL of H₂O and extracted with twice with dichloromethane. The combined organic layers were washed with brine. The mixture was dried over anhydrous sodium sulfate and concentrated under reduced pressure to give 8a (4 g, 92%) as brown oil, which was used without further purification.

Intermediate 8b 1-hydroxycyclopentanecarboxylic acid: 8a (3 g, 16.36 mmol, 1.00 equiv) was dissolved in acetic acid (4 mL) and concentrated hydrogen chloride (4 mL). The resulting solution was stirred for 4 h at 80° C. The mixture was then concentrated under reduced pressure to give 8b (2 g, 94%) as a white solid, which was used without further purification.

Intermediate 8c (4-cyclopropyl-3,4-dihydroquinoxalin-1(2H)-yl)(1-hydroxycyclopentyl)methanone: 8b (170 mg, 0.98 mmol), 1-hydroxycyclopentane-1-carboxylic acid (260 mg, 2.00 mmol), EDCI (288 mg, 1.50 mmol), and HOAT (204 mg, 1.50 mmol) were dissolved in N,N-dimethylformamide (3 mL) and stirred overnight at room temperature. The resulting solution was diluted with 10 mL of H₂O and extracted twice with ethyl acetate and the combined organic layers washed with brine. The organic phase was dried over anhydrous sodium sulfate and concentrated under reduced pressure. The resulting residue was purified by flash-column chromatography using ethyl acetate/petroleum ether (1:5) as eluent to give 8c (40 mg, 14%) as a yellow solid. MS (ES, m/z): 287 [M+H]⁺.

Example 8 (4-cyclopropyl-3,4-dihydroquinoxalin-1(2H)-yl)(1-(2,5dichlorobenzyloxy)cyclopentyl)methanone: 8c (30 mg, 0.10 mmol was dissolved in N,N-dimethylformamide (1 mL) and the resulting solution cooled to 0° C. To the stirring solution was added sodium hydride (10 mg, 0.25 mmol) and the resulting mixture was stirred for 10 min at 0° C. A solution of 2-(bromomethyl)-1,4-dichlorobenzene (40 mg, 0.17 mmol) in N,N-dimethylformamide (1 mL) was then added and the resulting solution was stirred for 4 h at room temperature. The crude mixture was purified by preparative HPLC with a C18 silica gel stationary phase using a 6 min gradient CH₃CN:H₂O 0.05% TFA (72:28 to 84:16) and detection by UV at 254 nm to provide the title compound TFA salt (25.7 mg, 55%) as a yellow semi-solid. MS (ES, m/z): 445 [M+1]⁺. ¹H-NMR (300 MHz, CD₃OD) δ 7.25-7.38 (m, 4H), 7.01-7.07 (m, 2H), 6.71 (t, J=7.8 Hz, 1H), 4.44 (s, 2H), 4.01 (s, 2H), 3.33 (m, 2H), 2.33-2.41 (m, 3H), 2.04-2.19 (m, 2H), 1.72-1.82 (m, 4H), 0.76 (m, 2H), 0.52 (m, 2H).

### Example 9

### (4-cyclopropyl-3,4-dihydroquinoxalin-1(2H)-yl)(1-(2,5-dichlorobenzyloxyl)cyclopropyl)methanone

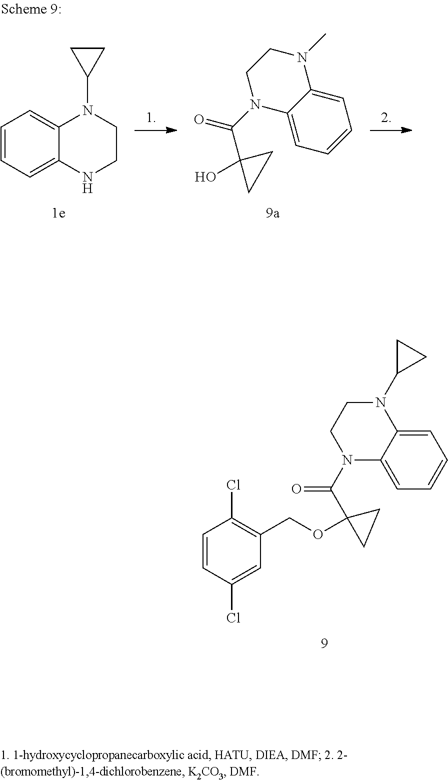 [SMILES: c1ccc2c(c1)NCCN2C1CC1 ; CN1CCN(C(=O)C2(O)CC2)c2ccccc21 ; O=C(N1CCN(C2CC2)c2ccccc21)C1(OCc2cc(Cl)ccc2Cl)CC1]

Intermediate 9a (4-cyclopropyl-3,4-dihydroquinoxalin-1(2H)-yl)(1-hydroxycyclopropyl)methanone: 1-Hydroxycyclopropane-1-carboxylic acid (100 mg, 0.98 mmol), 1-cyclopropyl-1,2,3,4-tetrahydroquinoxaline (100 mg, 0.57 mmol, 1.0 equiv), HATU (262 mg, 0.69 mmol, 1.2 equiv), DIEA (90 mg, 0.70 mmol) was dissolved in N,N-dimethylformamide (2 mL). The resulting solution was stirred overnight at room temperature, then diluted with 10 mL of H₂O and extracted twice with ethyl acetate. The organic layers were combined and washed with brine, then dried over anhydrous sodium sulfate and concentrated under reduced pressure. The residue was purified by preparative TLC with ethyl acetate/petroleum ether (1:1) to give 9a (100 mg, 67%) as a light yellow solid. MS (ES, m/z): 259 [M+1]⁺.

Example 9 (4-cyclopropyl-3,4-dihydroquinoxalin-1(2H)-yl)(1-(2,5-dichlorobenzyloxyl)cyclopropyl)methanone: 9a (85 mg, 0.33 mmol), 2-(bromomethyl)-1,4-dichlorobenzene (85 mg, 0.35 mmol), and potassium carbonate (85 mg, 0.62 mmol) were dissolved in N,N-dimethylformamide (2 mL). The resulting solution was stirred overnight at room temperature, then diluted with 20 mL of H₂O and extracted twice with ethyl acetate. The organic layers were combined and washed with brine, then dried over sodium sulfate and concentrated under reduced pressure. The crude mixture was purified by preparative HPLC with a C18 silica gel stationary phase using a 7 min gradient (CH₃CN:H₂O 0.05% TFA 60:40 to 80:20%) and detection by UV at 254 nm to provide the title compound (25.1 mg, 18%) as the TFA salt. MS (ES, m/z): 417 [M+H]⁺. ¹H-NMR (400 MHz, CD₃OD) δ 7.33 (d, J=8.0 Hz, 1H), 7.27 (d, J=8.4 Hz, 1H), 7.19 (d, J=8.4 Hz, 1H), 7.08 (t, J=8.0 Hz, 1H), 6.99 (d, J=7.6 Hz, 1H), 6.74 (t, J=8.0 Hz, 1H), 6.57 (m, 1H), 4.37 (s, 2H), 3.92 (s, 2H), 3.38-3.41 (m, 2H), 2.25-2.27 (m, 1H), 1.46 (m, 2H), 1.18-1.22 (m, 2H), 0.66-0.67 (m, 2H), 0.19 (m, 2H).

### Example 10

### (S)-(1-(2,5-dichlorobenzyl)pyrrolidin-2-yl)(3,4-dihydroquinolin-1(2H)-yl)methanone

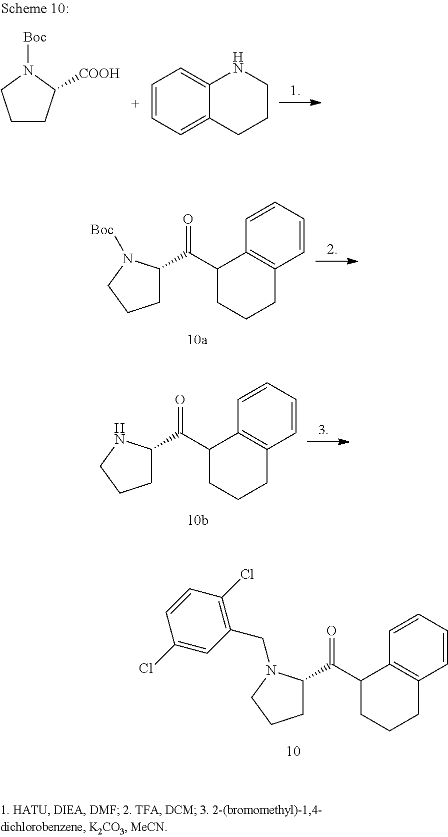

Intermediate 10a (S)-tert-butyl 2-(1,2,3,4-tetrahydroquinoline-1-carbonyl)pyrrolidine-1-carboxylate: (2 S)-1-[Tert-butoxy)carbonyl]pyrrolidine-2-carboxylic acid (500 mg, 2.32 mmol), 1,2,3,4-tetrahydroquinoline (620 mg, 4.65 mmol), HATU (1.77 g, 4.66 mmol), and DIEA (600 mg, 4.64 mmol) were dissolved in N,N-dimethylformamide (5 mL). The resulting solution was stirred for 2 h at room temperature, then quenched by the addition of water. The resulting solution was extracted with thrice with ethyl acetate and the organic layers combined and dried over anhydrous sodium sulfate and concentrated under reduced pressure to give 10a (0.57 g, 74%) as yellow oil, which was used directly without further purification.

Intermediate 10b (S)-(3,4-dihydroquinolin-1(2H)-yl)(pyrrolidin-2-yl)methanone: 10a (500 mg, 1.51 mmol) was dissolved in dichloromethane (10 mL) and trifluoroacetic acid (0.5 mL). The resulting solution was stirred overnight at room temperature, then concentrated under reduced pressure to give 10b (304 mg, 87%) as yellow oil, which was used directly without further purification.

Example 10 (S)-(1-(2,5-dichlorobenzyl)pyrrolidin-2-yl)(3,4-dihydroquinolin-1(2H)-yl)methanone: 10b (200 mg, 0.87 mmol), 2-(bromomethyl)-1,4-dichlorobenzene (208 mg, 0.87 mmol), and potassium carbonate (360 mg, 2.60 mmol) were dissolved in acetonitrile (5 mL) and the resulting solution was stirred overnight at room temperature. The mixture was then diluted with H₂O and extracted trice with ethyl acetate. The organic layers were combined and dried over anhydrous sodium sulfate, then concentrated under reduced pressure. The crude mixture was purified by preparative HPLC with a C18 silica gel stationary phase using a 8 min gradient (CH₃CN:H₂O 0.05% TFA 23:77 to 41:59) and detection by UV at 254 nm to provide the title compound as TFA salt (140 mg, 41%) as a yellow semi-solid. MS (ES, m/z): 389 [M+H]⁺. ¹H-NMR (400 MHz, CD₃OD) δ 7.75 (d, J=26.1 Hz, 1H), 7.54 (d, J=4.5 Hz, 2H), 7.33 (s, 2H), 7.14-7.23 (m, 2H), 4.55-4.72 (m, 2H), 3.79-3.90 (m, 1H), 3.70 (d, J=4.5 Hz, 2H), 3.50 (s, 1H), 3.37-3.42 (m, 1H), 2.84 (d, J=18 Hz, 1H), 2.68 (d, J=3.6 Hz, 1H), 2.21-2.33 (m, 1H), 2.01-2.15 (m, 4H), 1.74 (s, 1H).

### Example 11

### (S)-(1-(2,5-dichlorobenzyl)piperidin-2-yl)(3,4-dihydroquinolin-1(2H)-yl)methanone

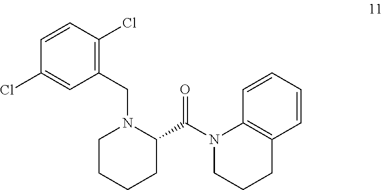 [SMILES: O=C([C@@H]1CCCCN1Cc1cc(Cl)ccc1Cl)N1CCCc2ccccc21]

Example 11: (S)-(1-(2,5-dichlorobenzyl)piperidin-2-yl)(3,4-dihydroquinolin-1(2H)-yl)methanone. 11 was synthesized in an analogous fashion to Example 10, using (S)-1-(tert-butoxycarbonyl)piperidine-2-carboxylic acid in place of (S)-tert-butyl 2-(1,2,3,4-tetrahydroquinoline-1-carbonyl)pyrrolidine-1-carboxylate. Isolated as the TFA salt. MS (ES, m/z): 403 [M+H]⁺. ¹H-NMR (300 MHz, CD₃OD) δ 7.80-7.86 (m, 1H), 7.54-7.61 (m, 2H), 7.37-7.41 (m, 3H), 7.19-7.22 (m, 1H), 4.76-4.81 (m, 1H), 4.66 (d, J=13.2 Hz, 1H), 4.51 (d, J=12.9 Hz, 1H), 4.36-4.40 (m, 1H), 3.46-3.55 (m, 1H), 2.82-2.94 (m, 1H), 2.12-2.19 (m, 1H), 1.88-1.99 (m, 2H), 1.68-1.77 (m, 4H), 1.33 (d, J=6.6 Hz, 1H).

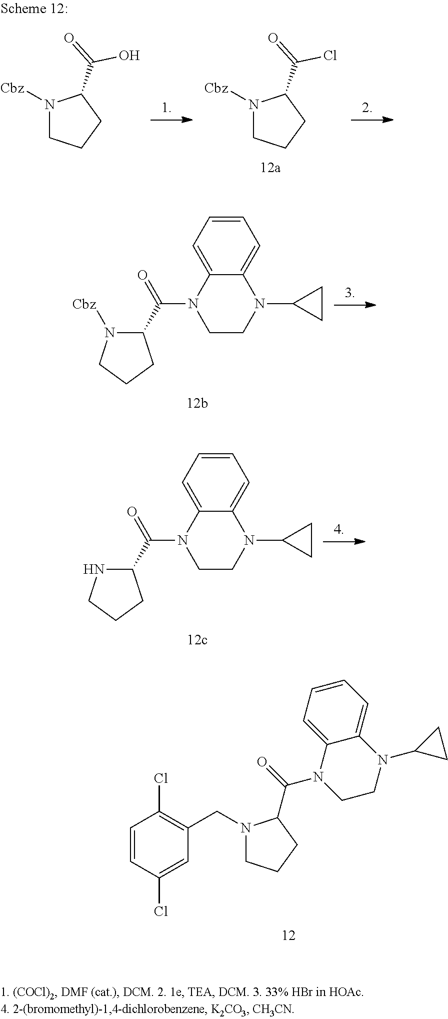 [SMILES: O=C(O)[C@@H]1CCCN1C(=O)OCc1ccccc1 ; O=C(Cl)[C@@H]1CCCN1C(=O)OCc1ccccc1 ; O=C([C@@H]1CCCN1C(=O)OCc1ccccc1)N1CCN(C2CC2)c2ccccc21 ; O=C([C@@H]1CCCN1)N1CCN(C2CC2)c2ccccc21 ; O=C(C1CCCN1Cc1cc(Cl)ccc1Cl)N1CCN(C2CC2)c2ccccc21]

Intermediate 12a: (S)-benzyl 2-(chlorocarbonyl)pyrrolidine-1-carboxylate. To a solution of (S)-1-[(benzyloxy)carbonyl]pyrrolidine-2-carboxylic acid (214 mg, 0.86 mmol, 1.00 equiv) and DMF (cat.) in DCM (10 mL) was added oxalyl chloride (324 mg, 2.55 mmol, 2.97 equiv) dropwise. The reaction mixture was stirred at room temperature for 2 h, and concentrated under reduced pressure to give 250 mg (crude) of (S)-benzyl 2-(chlorocarbonyl)pyrrolidine-1-carboxylate as yellow oil.

Intermediate 12b: (S)-benzyl 2-(4-cyclopropyl-1,2,3,4-tetrahydroquinoxaline-1-carbonyl) pyrrolidine-1-carboxylate. To a solution of 1e (210 mg, 1.21 mmol, 1.00 equiv) in DCM (20 mL), were added (S)-benzyl 2-(chlorocarbonyl)pyrrolidine-1-carboxylate (320 mg, 1.20 mmol, 1.00 equiv) and triethylamine (126 mg, 1.25 mmol, 1.00 equiv). The resulting solution was stirred for 4 h at room temperature. The resulting mixture was concentrated under reduced pressure to provide 400 mg (82%) of (S)-benzyl 2-(4-cyclopropyl-1,2,3,4-tetrahydroquinoxaline-1-carbonyl) pyrrolidine-1-carboxylate as a yellow solid. MS (ES, m/z): 406 [M+H]⁺.

Intermediate 12c: (S)-(4-cyclopropyl-3,4-dihydroquinoxalin-1(2H)-yl)(pyrrolidin-2-yl)methanone. To (S)-benzyl 2-(4-cyclopropyl-1,2,3,4-tetrahydroquinoxaline-1-carbonyl) pyrrolidine-1-carboxylate (300 mg, 0.74 mmol, 1.00 equiv) was added hydrogen bromide (33 wt % solution in glacial acetic acid, 5 mL). The mixture was stirred for 0.5 h at room temperature. The resulting mixture was concentrated under reduced pressure to give 300 mg (crude) of (S)-(4-cyclopropyl-3,4-dihydroquinoxalin-1(2H)-yl)(pyrrolidin-2-yl)methanone as a light yellow solid. MS (ES, m/z): 272 [M+H]⁺.

Example 12: (S)-(4-cyclopropyl-3,4-dihydroquinoxalin-1(2H)-yl)(1-(2,5-dichlorobenzyl) pyrrolidin-2-yl)methanone. To a solution of (S)-(4-cyclopropyl-3,4-dihydroquinoxalin-1(2H)-yl)(pyrrolidin-2-yl)methanone (50 mg, 0.18 mmol, 1.0 equiv) in CH₃CN (2 mL) were added 2-(bromomethyl)-1,4-dichlorobenzene (50 mg, 0.21 mmol, 1.2 equiv) and potassium carbonate (54 mg, 0.39 mmol, 2.0 equiv). The resulting solution was stirred for 2 h at room temperature. The resulting mixture was diluted with ethyl acetate, washed with brine (2×20 mL), dried over anhydrous sodium sulfate and concentrated under reduced pressure. The crude product (80 mg) was purified by Prep-HPLC:Column, SunFire Prep-C18, 19*150 mm 5 um; mobile phase gradient, water 0.05% TFA:CH₃CN (35% to 55% CH₃CN over 10 min; detector, Waters 2545 UV detector 254/220 nm) to provide 50 mg (63%) of the title compound as a white solid. MS (ES, m/z): 430 [M+H]⁺. ¹H-NMR (400 MHz, CD₃OD) δ 7.76 (s, 0.3H), 7.71 (s, 0.7H), 7.57-7.46 (m, 2H), 7.41 (d, J=8.9 Hz, 0.3H), 7.30-7.20 (m, 1.7H), 7.08 (d, J=7.8 Hz, 1H), 6.79 (t, J=7.1 Hz, 0.7H), 6.70-6.61 (m, 0.3H), 4.67-4.44 (m, 2H), 4.03-3.92 (m, 1H), 3.84-3.71 (m, 1H), 3.71-3.53 (m, 2H), 3.52-3.35 (m, 2H), 3.22-3.10 (m, 1H), 2.53-1.74 (m, 5H), 0.99-0.81 (m, 2H), 0.74-0.46 (m, 2H).

### Example 13

### (S)-(2-(4-cyclopropyl-1,2,3,4-tetrahydroquinoxaline-1-carbonyl)piperidin-1-yl)(2,5-dichlorophenyl)methanone

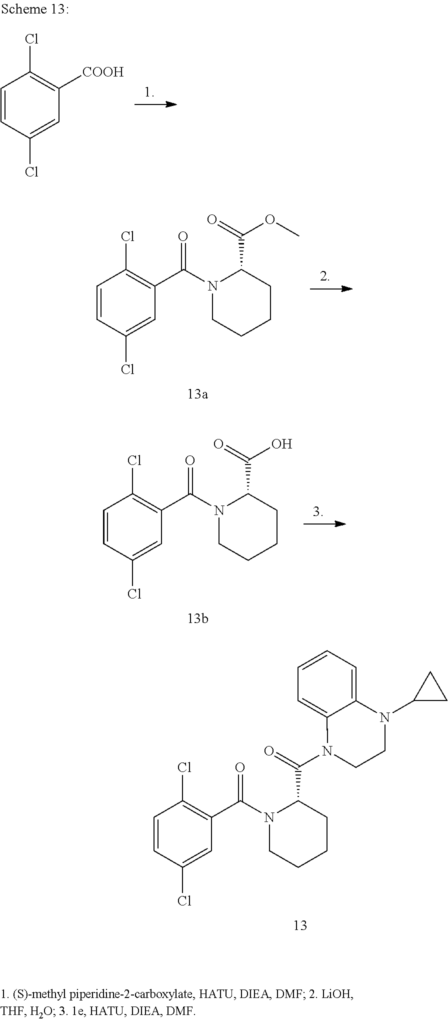 [SMILES: O=C(O)c1cc(Cl)ccc1Cl ; COC(=O)[C@@H]1CCCCN1C(=O)c1cc(Cl)ccc1Cl ; O=C(O)[C@@H]1CCCCN1C(=O)c1cc(Cl)ccc1Cl ; O=C([C@@H]1CCCCN1C(=O)c1cc(Cl)ccc1Cl)N1CCN(C2CC2)c2ccccc21]

Intermediate 13a: (S)-methyl 1-(2,5-dichlorobenzoyl)piperidine-2-carboxylate. To a solution of 2,5-dichlorobenzoic acid (1.00 g, 5.24 mmol, 1.00 equiv) in DMF (10 mL) were added (S)-methyl piperidine-2-carboxylate (750 mg, 5.24 mmol, 1.00 equiv), HATU (4.00 g, 10.5 mmol, 2.00 equiv), DIEA (2.74 g, 21.20 mmol, 4.00 equiv). The resulting solution was stirred overnight at room temperature. The resulting mixture was concentrated under reduced pressure. The residue was purified by flash-column chromatography with petroleum ether/ethyl acetate (1:1) to give 1.45 g (88%) of (S)-methyl 1-(2,5-dichlorobenzoyl)piperidine-2-carboxylate as yellow oil. MS (ES, m/z): 316 [M+H]⁺.

Intermediate 13b: (S)-1-(2,5-dichlorobenzoyl)piperidine-2-carboxylic acid. To a solution of (S)-methyl 1-(2,5-dichlorobenzoyl)piperidine-2-carboxylate (450 mg, 1.42 mmol, 1.00 equiv) in THF/water (10/10 mL) was added LiOH.H₂O (300 mg, 7.15 mmol, 5.00 equiv). The resulting solution was stirred overnight at room temperature. The resulting mixture was concentrated under reduced pressure. The pH of the solution was adjusted to 2-3 with hydrogen chloride (1 M). The resulting solution was extracted with ethyl acetate (3×30 mL). The organic layers were combined, dried over anhydrous sodium sulfate and concentrated under reduced pressure to give 0.4 g (93%) of (S)-1-(2,5-dichlorobenzoyl)piperidine-2-carboxylic acid as light-yellow oil. MS (ES, m/z): 302 [M+H]⁺.

Example 13: (S)-(2-(4-cyclopropyl-1,2,3,4-tetrahydroquinoxaline-1-carbonyl)piperidin-1-yl)(2,5-dichlorophenyl)methanone. To a solution of (S)-1-(2,5-dichlorobenzoyl)piperidine-2-carboxylic acid (130 mg, 0.43 mmol, 1.5 equiv) in DMF (5 mL) were added 1e (50 mg, 0.29 mmol, 1.0 equiv), HATU (218 mg, 0.57 mmol, 2.0 equiv) and DIEA (149 mg, 1.15 mmol, 4.00 equiv). The resulting solution was stirred overnight at room temperature. The resulting mixture was concentrated under reduced pressure. The crude product (100 mg) was purified by Prep-HPLC:Column, SunFire Prep-C18, 19*150 mm Sum; mobile phase gradient, water 0.05% TFA:CH₃CN (75% to 78% CH₃CN over 10 min; detector, Waters 2545 UV detector 254/220 nm) to provide 20 mg (15%) of (S)-(2-(4-cyclopropyl-1,2,3,4-tetrahydroquinoxaline-1-carbonyl)piperidin-1-yl)(2,5-dichlorophenyl)methanone TFA salt as a light yellow solid. MS (ES, m/z): 458 [M+H]⁺. ¹H-NMR (300 MHz, CD₃OD) δ 7.20-7.42 (m, 2H), 6.96-7.17 (m, 3H), 6.65 (t, J=7.5 Hz, 1H), 5.70-5.80 (m, 1H), 4.19-4.75 (m, 2H), 3.21-3.76 (m, 3H), 2.39 (d, J=3.6 Hz, 1H), 1.20-1.75 (m, 7H), 0.70-0.79 (m, 2H), 0.53-0.58 (m, 2H).

### Example 14

### (S)-(4-cyclopropyl-3,4-dihydroquinoxaline-1(2H)-yl)(1-(2,5-dichlorobenzyl)piperidin-2-yl) methanone

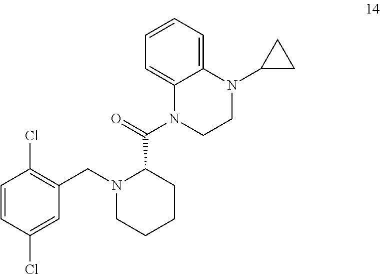 [SMILES: O=C([C@@H]1CCCCN1Cc1cc(Cl)ccc1Cl)N1CCN(C2CC2)c2ccccc21]

Example 14: (S)-(4-cyclopropyl-3,4-dihydroquinoxaline-1(2H)-yl)(1-(2,5-dichlorobenzyl) piperidin-2-yl)methanone. Example 14 was prepared using the procedure described for the preparation of Example 12, except that (S)-1-(t-butoxycarbonyl)piperidine-2-carboxylic acid was used in place of (S)-1-[(benzyloxy)carbonyl]pyrrolidine-2-carboxylic acid. Isolated as the bis TFA salt. MS (ES, m/z): 444 [M+H]⁺. ¹H-NMR (300 MHz, CD₃OD) δ 7.81 (s, 1H), 7.60-7.53 (m, 2H), 7.30-7.27 (m, 3H), 6.87-6.71 (m, 1H), 4.85-4.41 (m, 4H), 3.51-3.25 (m, 5H), 2.56-2.42 (m, 1H), 1.98-1.60 (m, 5H), 1.40-1.20 (m, 1H), 0.97-0.82 (m, 2H), 0.66-0.45 (m, 2H).

### Example 15

### (R)-(4-cyclopropyl-3,4-dihydroquinoxalin-1(2H)-yl)(1-(2,5-dichlorobenzyl)pyrrolidin-2-yl)methanone

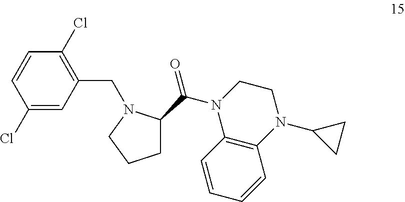 [SMILES: O=C([C@H]1CCCN1Cc1cc(Cl)ccc1Cl)N1CCN(C2CC2)c2ccccc21]

Example 15: (R)-(4-cyclopropyl-3,4-dihydroquinoxalin-1(2H)-yl)(1-(2,5-dichlorobenzyl) pyrrolidin-2-yl)methanone. Example 15 was prepared using the procedure described for the preparation of Example 12 except that (R)-1-(t-butoxycarbonyl)pyrrolidine-2-carboxylic acid was used in place of (S)-1-[(benzyloxy)carbonyl]pyrrolidine-2-carboxylic acid. Isolated as the bis TFA salt. MS (ES, m/z): 430 [M+H]⁺. ¹H-NMR (300 MHz, CD₃OD) δ 7.70-7.73 (m, 1H), 7.50-7.54 (m, 2H), 7.24-7.27 (m, 2H), 7.07-7.10 (m, 1H), 6.79-6.81 (m, 1H), 4.58 (dd, J=25, 13 Hz, 2H), 3.96-4.01 (m, 1H), 3.37-3.84 (m, 5H), 3.13-3.18 (m, 1H), 1.75-2.58 (m, 5H), 0.53-0.92 (m, 4H).

### Example 16

### (R)-(4-cyclopropyl-3,4-dihydroquinoxaline-1(2H)-yl)(1-(2,5-dichlorobenzyl)piperidin-2-yl) methanone

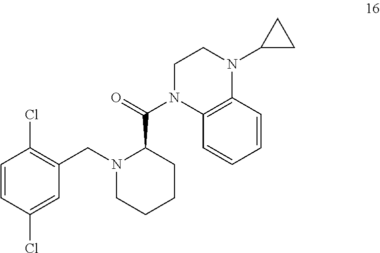 [SMILES: O=C([C@H]1CCCCN1Cc1cc(Cl)ccc1Cl)N1CCN(C2CC2)c2ccccc21]

Example 16: (R)-(4-cyclopropyl-3,4-dihydroquinoxaline-1 (2H)-yl)(1-(2,5-dichlorobenzyl) piperidin-2-yl) methanone. Example 16 was prepared using the procedure described for the preparation of Example 12, except that (R)-1-(t-butoxycarbonyl)piperidine-2-carboxylic acid was used in place of (S)-1-[(benzyloxy)carbonyl]pyrrolidine-2-carboxylic acid. Isolated as the bis TFA salt. MS (ES, m/z): 444 [M+H]⁺. ¹H-NMR (400 MHz, CD₃OD) δ 7.77-7.79 (m, 1H), 7.53-7.58 (m, 2H), 7.23-7.31 (m, 3H), 6.75-6.90 (m, 1H), 4.49-4.54 (m, 1H), 4.34-4.39 (m, 3H), 3.47-3.56 (m, 4H), 3.12-3.31 (m, 1H), 2.51 (m, 1H), 1.68-1.78 (m, 5H), 1.25-1.40 (m, 1H), 0.88-0.91 (m, 2H), 0.62-0.65.

### Example 17

### (S)-(4-cyclopropyl-3,4-dihydroquinoxaline-1(2H)-yl)(1-(2,5-dichlorobenzyl)azetidin-2-yl) methanone

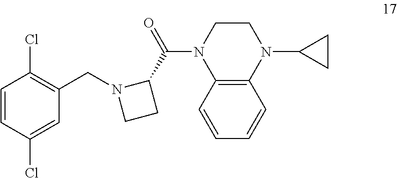 [SMILES: O=C([C@@H]1CCN1Cc1cc(Cl)ccc1Cl)N1CCN(C2CC2)c2ccccc21]

Example 17: (S)-(4-cyclopropyl-3,4-dihydroquinoxaline-1(2H)-yl)(1-(2,5-dichlorobenzyl) azetidin-2-yl)methanone. Example 17 was prepared using the procedure described for the preparation of Example 12 except that (S)-1-(t-butoxycarbonyl)azetidine-2-carboxylic acid was used in place of (S)-1-[(benzyloxy)carbonyl]pyrrolidine-2-carboxylic acid. Isolated as the bis TFA salt. MS (ES, m/z): 416 [M+H]⁺. ¹H-NMR (400 MHz, CD₃OD) δ 7.61 (s, 1H), 7.54 (m, 2H), 7.28-7.24 (m, 2H), 6.94 (d, J=8 Hz, 1H), 6.80 (m, 1H), 5.60 (t, J=9 Hz, 1H), 4.53 (dd, J=51, 14 Hz, 2H), 4.18 (m, 1H), 3.95 (m, 1H), 3.83-3.74 (m, 2H), 3.44-3.38 (m, 1H), 3.20-3.14 (m, 1H), 2.51 (s, 1H), 2.43-2.36 (m, 2H), 0.93 (d, J=6 Hz, 2H), 0.68-0.60 (m, 2H).

### Example 18

### (S)-(4-cyclopropyl-3,4-dihydroquinoxaline-1(2H)-yl)(1-(2,5-dichlorophenyl)sulfonyl)pyrrolidin-2-yl)methanone

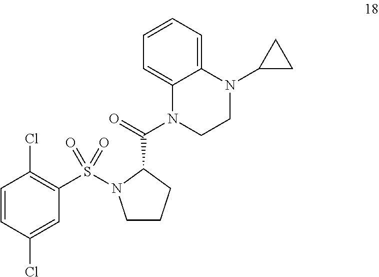 [SMILES: O=C([C@@H]1CCCN1S(=O)(=O)c1cc(Cl)ccc1Cl)N1CCN(C2CC2)c2ccccc21]

Example 18: (S)-(4-cyclopropyl-3,4-dihydroquinoxaline-1(2H)-yl)(1-(2,5-dichlorophenyl) sulfonyl)pyrrolidin-2-yl)methanone. To a solution of 2,5-dichlorobenzene-1-sulfonyl chloride (200 mg, 0.81 mmol, 1.00 equiv) in DCM (10 mL) were added 12c (220 mg, 0.81 mmol, 1.00 equiv) and triethylamine (180 mg, 1.78 mmol, 2.18 equiv). The resulting solution was stirred overnight at room temperature. The resulting mixture was concentrated under reduced pressure. The crude product (320 mg) was purified by Prep-HPLC:Column, SunFire Prep-C18, 19*150 mm Sum; mobile phase gradient, water 0.05% TFA:CH₃CN (46% to 61% CH₃CN over 7 min; detector, Waters 2545 UV detector 254/220 nm) to provide 191.8 mg (49%) of the title compound TFA salt as a brown solid. MS (ES, m/z): 480 [M+H]⁺. ¹H-NMR (300 MHz, CD₃OD) δ 7.72 (s, 1H), 7.54-7.53 (m, 2H), 7.25-7.16 (m, 2H), 6.98 (d, J=7.5 Hz, 1H), 6.68-6.62 (m, 1H), 5.09-4.96 (m, 1H), 4.30-4.19 (m, 1H), 3.70-3.64 (m, 1H), 3.50-3.35 (m, 4H), 2.53 (s, 1H), 2.18-2.01 (m, 3H), 1.88-1.74 (m, 1H), 0.88-0.84 (m, 2H), 0.72-0.62 (m, 2H).

### Example 19

### (S)-(4-cyclopropyl-3,4-dihydroquinoxaline-1 (2H)-yl)(1-(2,5-dichlorobenzyl)-4,4-dimethylpyrrolidin-2-yl)methanone

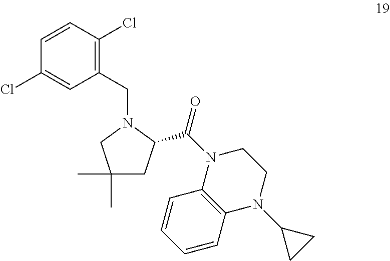 [SMILES: CC1(C)C[C@@H](C(=O)N2CCN(C3CC3)c3ccccc32)N(Cc2cc(Cl)ccc2Cl)C1]

Example 19: (S)-(4-cyclopropyl-3,4-dihydroquinoxaline-1 (2H)-yl)(1-(2,5-dichlorobenzyl)-4,4-dimethylpyrrolidin-2-yl)methanone. Example 19 was prepared using the procedure described for the preparation of Example 12 except that (S)-1-(t-butoxycarbonyl)-4,4-dimethylpyrrolidine-2-carboxylic acid was used in place of (S)-1-[(benzyloxy) carbonyl]pyrrolidine-2-carboxylic acid. Isolated as the bis TFA salt. MS (ES, m/z): 458 [M+H]⁺. ¹H-NMR (300 MHz, CD₃OD) δ 7.77 (m, 1H), 7.53 (m, 2H), 7.27 (m, 2H), 7.05 (m, 1H), 6.80 (m, 1H), 4.97 (m, 1H), 4.59 (m, 2H), 3.70 (m, 2H), 3.56 (m, 1H), 3.42 (m, 2H), 3.05 (m, 1H), 2.51 (m, 1H), 2.13 (m, 1H), 1.75 (m, 1H), 1.17 (m, 6H), 0.93 (m, 2H), 0.68 (m, 1H), 0.55 (m, 1H).

### Example 20

### (S)-(4-cyclopropyl-3,4-dihydroquinoxaline-1(2H)-yl)(1-(2,5-dichlorobenzyl)-2-methylpyrrolidin-2-yl)methanone

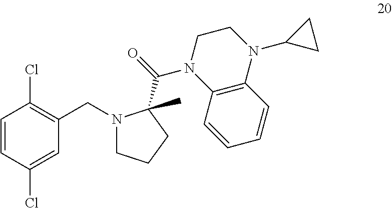 [SMILES: C[C@@]1(C(=O)N2CCN(C3CC3)c3ccccc32)CCCN1Cc1cc(Cl)ccc1Cl]

Example 20: (S)-(4-cyclopropyl-3,4-dihydroquinoxaline-1 (2H)-yl)(1-(2,5-dichlorobenzyl)-2-methylpyrrolidin-2-yl)methanone. Example 20 was prepared using the procedure described for the preparation of Example 12 except that (S)-1-((benzyloxy)carbonyl)-2-methylpyrrolidine-2-carboxylic acid was used in place of (S)-1-[(benzyloxy) carbonyl]pyrrolidine-2-carboxylic acid. Isolated as the bis TFA salt. MS (ES, m/z): 444 [M+H]⁺. ¹H-NMR (300 MHz, CD₃OD) δ 7.96 (s, 1H), 7.59-7.51 (m, 2H), 7.38 (s, 1H), 7.28-7.23 (m, 2H), 6.82-6.76 (m, 1H), 4.72-4.62 (m, 1H), 4.41-4.02 (m, 2H), 3.80-3.32 (m, 5H), 2.53-2.14 (m, 4H), 2.10-1.95 (m, 1H), 1.80-1.38 (m, 3H), 0.95-0.84 (m, 2H), 0.72-0.50 (m, 2H).

### Example 21

### (R)-(4-cyclopropyl-3,4-dihydroquinoxaline-1(2H)-yl)(3-(2,5-dichlorobenzyl)thiazolidin-4-yl)methanone

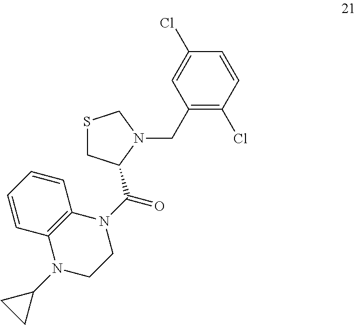 [SMILES: O=C([C@@H]1CSCN1Cc1cc(Cl)ccc1Cl)N1CCN(C2CC2)c2ccccc21]

Example 21: (R)-(4-cyclopropyl-3,4-dihydroquinoxaline-1(2H)-yl)(3-(2,5-dichlorobenzyl) thiazolidin-4-yl)methanone. Example 21 was prepared using the procedure described for the preparation of Example 12 except that (R)-3-(t-butoxycarbonyl)thiazolidine-4-carboxylic acid was used in place of (S)-1-[(benzyloxy)carbonyl]pyrrolidine-2-carboxylic acid. Isolated as the bis TFA salt. MS (ES, m/z): 448 [M+H]⁺. ¹H-NMR (300 MHz, CD₃OD) δ 7.40-7.03 (m, 6H), 6.70-6.63 (m, 1H), 4.74 (bs, 1H), 4.11 (d, J=9.9 Hz, 2H), 3.98-3.76 (m, 3H), 3.60-3.36 (m, 4H), 3.22-3.09 (m, 1H), 2.49-2.40 (m, 1H), 0.85-0.81 (m, 2H), 0.61-0.49 (m, 2H).

### Example 22

### (R)-(4-cyclopropyl-3,4-dihydroquinoxaline-1(2H)-yl)(4-(2,5-dichlorobenzyl)thiomorpholin-3-yl)methanone

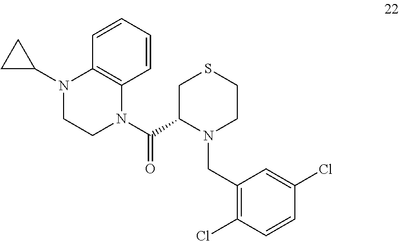 [SMILES: O=C([C@@H]1CSCCN1Cc1cc(Cl)ccc1Cl)N1CCN(C2CC2)c2ccccc21]

Example 22: (R)-(4-cyclopropyl-3,4-dihydroquinoxaline-1(2H)-yl)(4-(2,5-dichlorobenzyl)thiomorpholin-3-yl)methanone. Example 22 was prepared using the procedure described for the preparation of Example 12 except that (R)-4-(t-butoxycarbonyl)thiomorpholine-3-carboxylic acid was used in place of (S)-1-[(benzyloxy) carbonyl]pyrrolidine-2-carboxylic acid. MS (ES, m/z): 462 [M+H]⁺. ¹H-NMR (400 MHz, CDCl₃) δ 7.51 (s, 1H), 7.28-7.25 (m, 2H), 7.19-7.15 (m, 3H), 6.76-6.72 (m, 1H), 4.21-4.10 (m, 2H), 3.88-3.77 (m, 1H), 3.70-3.62 (m, 1H), 3.61-3.48 (m, 1H), 3.47-3.32 (m, 3H), 2.94-2.60 (m, 2H), 2.53-2.36 (m, 3H), 1.60-1.54 (m, 1H), 0.91-0.80 (m, 2H), 0.68-0.50 (m, 2H).

### Example 23

### (S)-(4-cyclopropyl-3,4-dihydroquinoxaline-1(2H)-yl)(4-(2,5-dichlorobenzyl)morpholin-3-yl)methanone

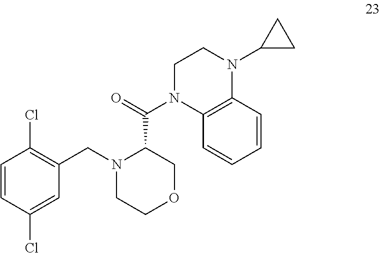 [SMILES: O=C([C@@H]1COCCN1Cc1cc(Cl)ccc1Cl)N1CCN(C2CC2)c2ccccc21]

Example 23: (S)-(4-cyclopropyl-3,4-dihydroquinoxaline-1(2H)-yl)(1-(2,5-dichlorobenzyl)-2-methylpyrrolidin-2-yl)methanone. Example 23 was prepared using the procedure described for the preparation of Example 12 except that (S)-4-(t-butoxycarbonyl)morpholine-3-carboxylic acid was used in place of (S)-1-[(benzyloxy) carbonyl]pyrrolidine-2-carboxylic acid. Isolated as the bis TFA salt. MS (ES, m/z): 446 [M+H]⁺. ¹H-NMR (400 MHz, CDCl₃) δ 7.71 (s, 1H), 7.43-7.35 (m, 2H), 7.21 (s, 2H), 6.72 (s, 2H), 4.66-4.62 (m, 2H), 4.47 (s, 1H), 4.39-4.34 (m, 1H), 4.10-3.30 (m, 8H), 3.02 (s, 1H), 2.48-2.45 (m, 1H), 0.95-0.82 (m, 2H), 0.74-0.67 (m, 1H), 0.55-0.46 (m, 1H).

### Example 24

### (4-cyclopropyl-3,4-dihydroquinoxaline-1(2H)-yl)((2S)-3-(2,5-dichlorobenzyl)-3-azabicyclo[3.1.0]hexan-2-yl)methanone

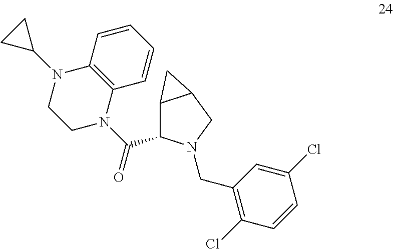 [SMILES: O=C([C@@H]1C2CC2CN1Cc1cc(Cl)ccc1Cl)N1CCN(C2CC2)c2ccccc21]

Example 24: (4-cyclopropyl-3,4-dihydroquinoxaline-1(2H)-yl)((2S)-3-(2,5-dichlorobenzyl)-3-azabicyclo[3.1.0]hexan-2-yl)methanone. Example 24 was prepared using the procedure described for the preparation of Example 12 except that (2S)-3-((benzyloxy)carbonyl)-3-azabicyclo[3.1.0]hexane-2-carboxylic acid (prepared from commercial (2S)-3-azabicyclo[3.1.0]hexane-2-carboxylic acid by the action of benzyl chloroformate under typical Schotten-Baumann conditions) was used in place of (S)-1-[(benzyloxy) carbonyl]pyrrolidine-2-carboxylic acid. MS (ES, m/z): 442 [M+H]⁺. ¹H-NMR (300 MHz, CD₃OD) δ 7.78-7.69 (m, 1H), 7.57-7.43 (m, 2H), 7.31-7.20 (m, 2.7H), 7.11-7.04 (m, 0.3H), 6.88-6.78 (m, 0.7H), 6.65-6.56 (m, 0.3H), 5.25 (d, J=4.5 Hz, 1H), 4.72-4.54 (m, 2H), 4.37-4.29 (m, 0.7H), 4.08-3.97 (m, 0.3H), 3.95-3.24 (m, 5H), 2.57-2.28 (m, 1.3H), 2.08-1.70 (m, 0.3H), 1.81-1.70 (m, 0.7H), 1.43-1.32 (m, 0.7H), 1.01-0.42 (m, 6H).

### Example 25

### (S)-5-(4-cyclopropyl-1,2,3,4-tetrahydroquinoxaline-1-carbonyl)-1-(2,5-dichlorobenzyl)pyrrolidin-2-one

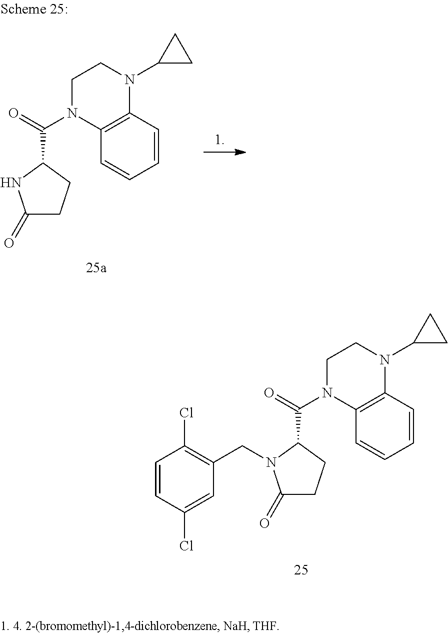 [SMILES: O=C1CC[C@@H](C(=O)N2CCN(C3CC3)c3ccccc32)N1 ; O=C([C@@H]1CCC(=O)N1Cc1cc(Cl)ccc1Cl)N1CCN(C2CC2)c2ccccc21]

Intermediate 25a: (S)-5-(4-cyclopropyl-1,2,3,4-tetrahydroquinoxaline-1-carbonyl)-pyrrolidin-2-one. Intermediate 25a was prepared using the procedure described for the preparation of Intermediate 12c except that (S)-1-(t-butoxycarbonyl)-5-oxopyrrolidine-2-carboxylic acid was used in place of (S)-1-[(benzyloxy) carbonyl]pyrrolidine-2-carboxylic acid.

Example 25: (S)-5-(4-cyclopropyl-1,2,3,4-tetrahydroquinoxaline-1-carbonyl)-1-(2,5-dichlorobenzyl)pyrrolidin-2-one. To a mixture of (S)-5-(4-cyclopropyl-1,2,3,4-tetrahydroquinoxaline-1-carbonyl)-pyrrolidin-2-one 25a (80 mg, 0.28 mmol, 1.0 equiv) in THF (5 mL) was added sodium hydride (33 mg, 0.82 mmol, 3.00 equiv, 60%), followed by addition of 2-(bromomethyl)-1,4-dichlorobenzene (67 mg, 0.28 mmol, 1.00 equiv). The resulting solution was stirred for 2 h at room temperature. The resulting solution was diluted with ethyl acetate (10 mL), washed with brine (2×10 mL) and concentrated under reduced pressure. The crude product (50 mg) was purified by Prep-HPLC:Column, SunFire Prep-C18, 19*150 mm Sum; mobile phase gradient, water 0.05% TFA:CH₃CN (56% to 70% CH₃CN over 10 min; detector, Waters 2545 UV detector 254 and 220 nm) to provide 20 mg (16%) of the title compound TFA salt as a white solid. MS (ES, m/z): 444 [M+H]⁺. ¹H-NMR (300 MHz, CD₃OD) δ 7.18 (m, 5H), 6.67 (m, 1H), 6.52 (m, 1H), 4.65 (m, 2H), 4.09 (m, 1H), 3.89 (m, 1H), 3.32 (m, 3H), 2.52 (m, 1H), 2.38 (m, 2H), 2.13 (m, 1H), 1.98 (m, 1H), 0.76 (m, 2H), 0.51 (m, 2H).

### Example 26

### (4-cyclopropyl-3,4-dihydroquinoxalin-1(2H)-yl)(1-((2,5-dichlorobenzyl)amino)cyclopropyl)methanone

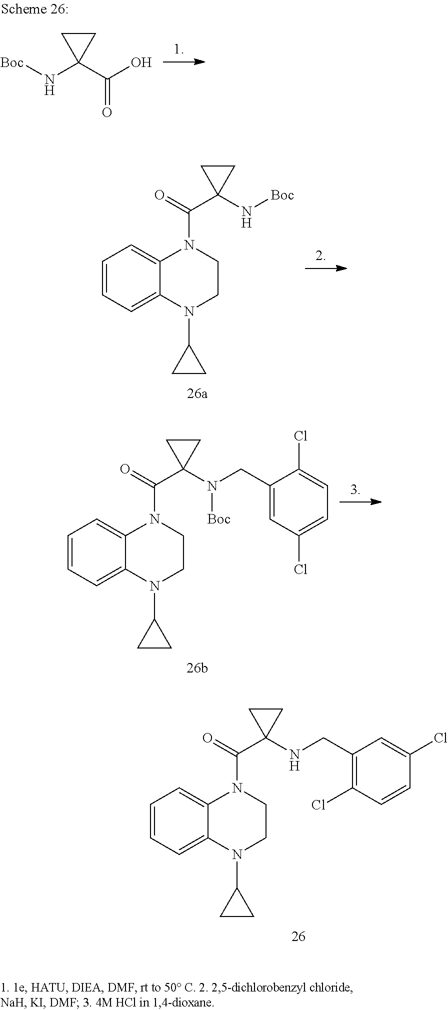 [SMILES: CC(C)(C)OC(=O)NC1(C(=O)O)CC1 ; CC(C)(C)OC(=O)NC1(C(=O)N2CCN(C3CC3)c3ccccc32)CC1 ; CC(C)(C)OC(=O)N(Cc1cc(Cl)ccc1Cl)C1(C(=O)N2CCN(C3CC3)c3ccccc32)CC1 ; O=C(N1CCN(C2CC2)c2ccccc21)C1(NCc2cc(Cl)ccc2Cl)CC1]

Intermediate 26a: t-Butyl (1-(4-cyclopropyl-1,2,3,4-tetrahydroquinoxaline-1-carbonyl)cyclopropyl)carbamate. To a mixture of Boc-1-aminocyclopropane-1-carboxylic acid (40 mg, 0.20 mmol, 1 equiv) and 1e (34.6 mg, 0.20 mmol, 1 equiv) in DMF (0.5 mL) were added DIEA (173 uL, 10 mmol, 5 equiv) and HATU (90.8 mg, 0.24 mmol, 1.2 equiv). The mixture was stirred at room temperature for 1 hr and at 50° C. overnight. The mixture was diluted with ethyl acetate, washed with H₂O (2×) and brine (1×), dried over anhydrous sodium sulfate, and concentrated under reduced pressure. The residue was purified by flash-column chromatography to give 40 mg (56%) t-Butyl(1-(4-cyclopropyl-1,2,3,4-tetrahydroquinoxaline-1-carbonyl)cyclopropyl)carbamate as a clear syrup. MS (ES, m/z): 357.9 [M+H]⁺.

Intermediate 26b: t-Butyl(1-(4-cyclopropyl-1,2,3,4-tetrahydroquinoxaline-1-carbonyl)cyclopropyl)(2,5-dichlorobenzyl)carbamate. To a solution of t-Butyl(1-(4-cyclopropyl-1,2,3,4-tetrahydroquinoxaline-1-carbonyl)cyclopropyl)carbamate (83.7 mg, 0.234 mmol, 1.00 equiv) in DMF (1 mL) at 0° C. was added 60% sodium hydride (103 mg, 0.258 mmol, 1.1 equiv). The mixture was stirred at room temperature for 30 minutes and cooled to 0° C. To the mixture was added a solution of 2,5-dichlorobenzyl chloride (49 uL, 0.35 mmol, 1.3 equiv) in DMF (0.4 mL) and KI (cat.). The mixture was stirred at room temperature for 2 h and 45° C. for 1 h. The reaction was quenched with water, extracted with ethyl acetate. The organic layer was washed with brine (1×), dried over anhydrous sodium sulfate, concentrated under reduced pressure, and purified by flash-column chromatography to give 82 mg (68%) t-Butyl(1-(4-cyclopropyl-1,2,3,4-tetrahydroquinoxaline-1-carbonyl)cyclopropyl)(2,5-dichlorobenzyl)carbamate as a yellow syrup. MS (ES, m/z): 515.9 [M+H]⁺.

Example 26: (4-cyclopropyl-3,4-dihydroquinoxalin-1(2H)-yl)(1-((2,5-dichlorobenzyl)amino)cyclopropyl)methanone. To t-Butyl(1-(4-cyclopropyl-1,2,3,4-tetrahydroquinoxaline-1-carbonyl)cyclopropyl)(2,5-dichlorobenzyl)carbamate (82 mg, 0.16 mmol) was added hydrochloric acid (4 M in 1,4-dioxane). The mixture was stirred at room temperature for 1 hour and concentrated. The residue was diluted with ethyl acetate, washed with saturated aqueous NaHCO₃ (1×) and brine (1×), dried over anhydrous sodium sulfate, concentrated under reduced pressure, and purified by flash-column chromatography to give 32 mg (48%) (4-cyclopropyl-3,4-dihydroquinoxalin-1(2H)-yl)(1-((2,5-dichlorobenzyl)amino)cyclopropyl)methanone as a clear syrup. MS (ES, m/z): 416 [M+H]⁺. ¹H-NMR (400 MHz, CD₃OD) δ 7.33 (dd, J=7.9, 1.5 Hz, 1H), 7.26 (d, J=8.5 Hz, 1H), 7.20 (dd, J=8.3, 1.4 Hz, 1H), 7.17 (dd, J=8.5, 2.6 Hz, 1H), 7.12 (ddd, J=8.4, 7.3, 1.5 Hz, 1H), 6.87 (s, 1H), 6.78-6.71 (m, 1H), 3.89 (t, J=5.8 Hz, 2H), 3.61 (s, 2H), 3.44 (t, J=5.9 Hz, 2H), 2.46-2.35 (m, 1H), 1.46-1.38 (m, 2H), 0.95 (q, J=4.3 Hz, 2H), 0.82-0.74 (m, 2H), 0.49-0.41 (m, 2H).

### Example 27

### (4-cyclopropyl-3,4-dihydroquinoxalin-1(2H)-yl)(1-((2,5-dichlorobenzyl)(methyl)amino)cyclopropyl)methanone

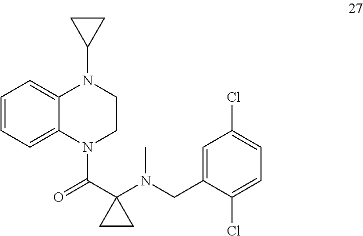 [SMILES: CN(Cc1cc(Cl)ccc1Cl)C1(C(=O)N2CCN(C3CC3)c3ccccc32)CC1]

Example 27: (4-cyclopropyl-3,4-dihydroquinoxalin-1(2H)-yl)(1-((2,5-dichlorobenzyl)(methyl)amino)cyclopropyl)methanone. To a mixture of (4-cyclopropyl-3,4-dihydroquinoxalin-1(2H)-yl)(1-((2,5-dichlorobenzyl)amino)cyclopropyl)methanone (14.5 mg, 0.035 mmol) in DMF (0.2 mL) were added iodomethane (14.4 uL, 0.23 mmol) and K₂CO₃ (12.8 mg, 0.093 mmol). The mixture was stirred at 50° C. for 64 hours and purified by Prep-HPLC to give 8 mg (4-cyclopropyl-3,4-dihydroquinoxalin-1(2H)-yl)(1-((2,5-dichlorobenzyl)(methyl)amino)cyclopropyl)methanone bis TFA salt as a yellow syrup. MS (ES, m/z): 430 [M+H]⁺. ¹H-NMR (400 MHz, CD₃OD) δ 7.35-7.29 (m, 2H), 7.27-7.22 (m, 2H), 7.18 (d, J=2.5 Hz, 1H), 7.05 (ddd, J=8.6, 7.3, 1.5 Hz, 1H), 6.70-6.63 (m, 1H), 3.99 (t, J=5.4 Hz, 2H), 3.80 (s, 2H), 3.41 (t, J=5.6 Hz, 2H), 2.49-2.33 (m, 1H), 2.19 (s, 3H), 1.29 (dd, J=7.6, 5.3 Hz, 2H), 1.15 (dd, J=7.7, 5.2 Hz, 2H), 0.88-0.77 (m, 2H), 0.64-0.53 (m, 2H).

### Example 28

### 2-(2-chlorobenzyl)pyrrolidin-1-yl)(4-cyclopropyl-3,4-dihydroquinoxalin-1(2H)-yl)methanone

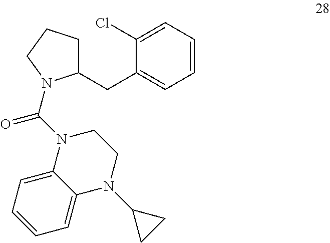 [SMILES: O=C(N1CCN(C2CC2)c2ccccc21)N1CCCC1Cc1ccccc1Cl]

Example 28: 2-(2-chlorobenzyl)pyrrolidin-1-yl)(4-cyclopropyl-3,4-dihydroquinoxalin-1(2H)-yl)methanone. To a solution of triphosgene (22.7 mg, 0.077 mmol, 1 equiv) in DCM (1.3 mL) at 0° C. was added a solution of 1e (40 mg, 0.23 mmol, 3 equiv) and triethylamine (40 uL, 0.29 mmol, 3.7 equiv) in DCM (1 mL). The mixture was stirred at room temperature for 2 h. To the mixture were added 2-(2-chloro-benzyl)-pyrrolidine (54 mg, 0.276 mmol, 3.6 equiv) and triethylamine (42 uL, 0.299 mmol, 3.9 equiv). The mixture was stirred at room temperature for 1 h, concentrated under reduced pressure, and purified by flash-column chromatography to give 62.8 mg (69%) as yellow syrup. MS (ES, m/z): 396 [M+H]⁺. ¹H-NMR (400 MHz, CDCl₃) δ 7.33 (dd, J=7.4, 1.8 Hz, 1H), 7.28-7.20 (m, 1H), 7.17-7.12 (m, 3H), 6.94 (dd, J=12.1, 4.6 Hz, 2H), 6.68 (td, J=7.5, 1.4 Hz, 1H), 4.35-4.26 (m, 1H), 4.20-4.06 (m, 1H), 3.49-3.31 (m, 3H), 3.32-3.05 (m, 3H), 2.80 (dd, J=12.9, 9.5 Hz, 1H), 2.47-2.31 (m, 1H), 1.82-1.74 (m, 2H), 1.70-1.56 (m, 2H), 0.86-0.76 (m, 2H), 0.76-0.66 (m, 1H), 0.57-0.43 (m, 1H).

### Example 29

### (4-cyclopropyl-3,4-dihydroquinoxalin-1(2H)-yl)(2-(2,5-dichlorophenoxy)cyclohex-1-enyl)methanone

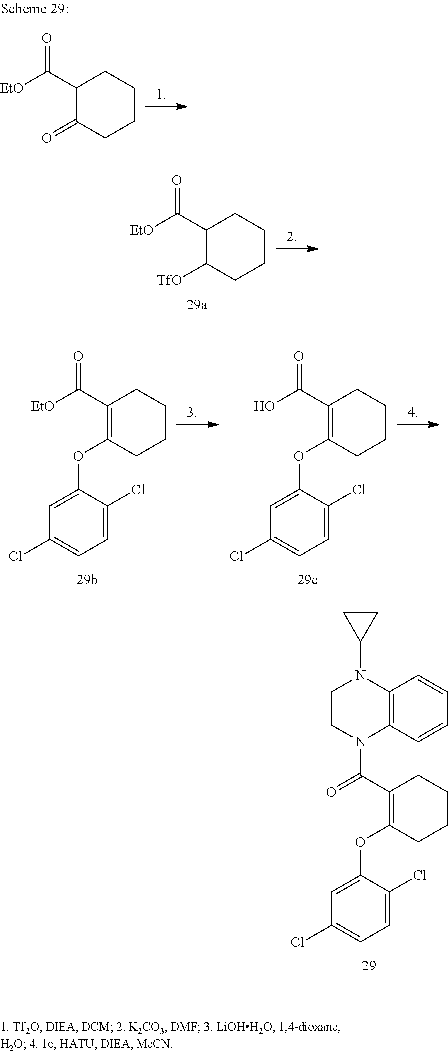 [SMILES: CCOC(=O)C1CCCCC1=O ; CCOC(=O)C1CCCCC1OS(=O)(=O)C(F)(F)F ; CCOC(=O)C1=C(Oc2cc(Cl)ccc2Cl)CCCC1 ; O=C(O)C1=C(Oc2cc(Cl)ccc2Cl)CCCC1 ; O=C(C1=C(Oc2cc(Cl)ccc2Cl)CCCC1)N1CCN(C2CC2)c2ccccc21]

Intermediate 29a: ethyl 2-(trifluoromethylsulfonyloxy)cyclohex-1-enecarboxylate. Ethyl 2-oxocyclohexanecarboxylate (170 mg, 1.00 mmol) and DIEA (417 μL, 2.40 mmol) were dissolved in DCM (2 mL) and cooled to −78° C. To the stirring solution was added dropwise trifluoromethanesulfonic anhydride (202 μL, 1.20 mmol), then the resulting solution was allowed to warm to room temperature and stirred for 16 h. The solution was then diluted with DCM and washed with 1M aqueous HCl and the solvent removed under reduced pressure. The crude residue was purified by flash column chromatography using a gradient of hexanes:EtOAc (9:1 to 1:1) to give 29a (231 mg, 76%) as a clear oil. ¹H-NMR (400 MHz, CDCl₃) δ 4.23 (q, J=7.1 Hz, 2H), 2.51-2.40 (m, 2H), 2.40-2.31 (m, 2H), 1.81-1.70 (m, 2H), 1.70-1.57 (m, 2H), 1.28 (t, J=7.1 Hz, 3H).

Intermediate 29b: ethyl 2-(2,5-dichlorophenoxy)cyclohex-1-enecarboxylate. 29a (174 mg, 0.576 mmol), K₂CO₃ (279 mg, 2.02 mmol), and DMF (2 mL) were combined and the resulting suspension stirred at 120° C. for 2 h. The suspension was diluted with MeOH and filtered, then the solvent removed under reduced pressure. The crude residue was purified by flash column chromatography using a gradient of hexanes:EtOAc (95:5 to 75:25) to give 29b (108 mg, 59%). ¹H-NMR (400 MHz, CDCl₃, mixture of rotamers) δ 7.29 (d, J=8.5 Hz, 1H), 7.19 (d, J=2.4 Hz, 0.33H), 7.00 (dd, J=8.5, 2.4 Hz, 0.33H), 6.94 (dd, J=8.5, 2.3 Hz, 0.67H), 6.85 (d, J=2.3 Hz, 0.67H), 4.18 (m, 0.67H), 4.07 (q, J=7.1 Hz, 0.33H), 2.46 (m, 2H), 2.22 (m, 2H), 2.19-1.92 (m, 2H), 1.80-1.55 (m, 4H), 1.26 (t, J=7.1 Hz, 1H), 1.08 (t, J=7.1 Hz, 2H).

Intermediate 29c: 2-(2,5-dichlorophenoxy)cyclohex-1-enecarboxylic acid. 29b (108 mg, 0.343 mmol) and LiOH.H₂O (115 mg, 2.74 mmol) were dissolved in EtOH (2 mL) and H₂O (1 mL) and stirred at 80° C. for 1 h. The solvent was removed under reduced pressure and the resulting residue dissolved in DCM and washed with 5% aqueous HCl, then the solvent removed to give 29c (23 mg, 23%) as a clear oil. ¹H NMR (400 MHz, CDCL1₃, mixture of rotamers) δ 7.34 (d, J=8.6 Hz, 0.67H), 7.30 (d, J=8.6 Hz, 0.33H), 7.19 (d, J=2.4 Hz, 0.33H), 7.09 (dd, J=8.6, 2.4 Hz, 0.67H), 7.04 (d, J=2.4 Hz, 0.33H), 7.03-6.99 (m, 0.67H), 2.47 (t, J=6.1 Hz, 1.34H), 2.14 (m, 2.68H), 1.79-1.59 (m, 4.02H).

Example 29: (4-cyclopropyl-3,4-dihydroquinoxalin-1(2H)-yl)(2-(2,5-dichlorophenoxy)cyclohex-1-enyl)methanone. 29c (23 mg, 0.080 mmol), le (17 mg, 0.096), HATU (34 mg, 0.088 mmol), and DIEA (56 μL, 0.32 mmol) were dissolved in MeCN (1 mL). The solution was stirred at room temperature for 1 h, then a single crystal of DMAP added and the solution stirred and additional 2 h at room temperature. The solution was then heated for 2 h at 60° C., then purified by preparative HPLC with a C18 silica gel stationary phase using a gradient of H₂O 0.05% TFA:CH₃CN 0.05% TFA (50:50 to 5:95) and detection by UV at 254 nm to give the title compound (3.2 mg, 9%) TFA salt as a yellow powder. MS (ES, m/z): 443 [M+1]⁺. ¹H-NMR (400 MHz, CD₃OD) δ 7.29 (d, J=8.4 Hz, 1H), 7.11 (t, J=7.1 Hz, 1H), 7.03-6.84 (m, 4H), 6.69 (t, J=7.1 Hz, 1H), 4.63 (s, 1H), 3.02 (s, 1H), 2.66 (s, 1H), 2.43 (s, 1H), 2.11 (s, 1H), 1.84-1.57 (m, 4H), 1.36-1.27 (m, 2H), 0.94-0.83 (m, 2H), 0.73-0.38 (m, 4H).

### Example 30

### (S)-(2H-benzo[b][1,4]thiazin-4(3H)-yl)(1-(2,5-dichlorobenzyl)pyrrolidin-2-yl)methanone

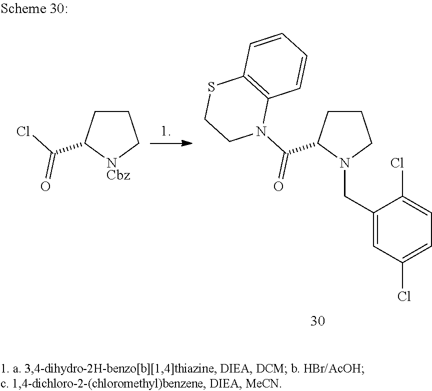 [SMILES: O=C(Cl)[C@@H]1CCCN1C(=O)OCc1ccccc1 ; O=C([C@@H]1CCCN1Cc1cc(Cl)ccc1Cl)N1CCSc2ccccc21]

Example 30: (S)-(2H-benzo[b][1,4]thiazin-4(3H)-yl)(1-(2,5-dichlorobenzyl)pyrrolidin-2-yl)methanone. 3,4-dihydro-2H-benzo[b][1,4]thiazine (67 mg, 0.44 mmol) and DIEA (209 μL, 1.21 mmol) were dissolved in DCM (1 mL) and cooled to 0° C. To the stirring solution was added dropwise, a solution of (S)-benzyl 2-(chlorocarbonyl)pyrrolidine-1-carboxylate (108 mg, 0.402 mmol) in DCM (1 mL). The resulting solution was then allowed to warm to room temperature and stirred for 1 h. The solvent was removed under reduced pressure and then further dried in vacuo. The crude residue was dissolved in 33 wt % hydrobromic acid in acetic acid solution and left at room temperature for 1 h, then triturated with Et₂O and the solvent decanted to give an oil. The crude oil was dissolved in MeCN, to which 1,4-dichloro-2-(chloromethyl)benzene (94 mg, 0.48 mmol) and DIEA (349 μL, 2.01 mmol). The solution was stirred at room temperature for 16 h, then purified by flash column chromatography using a gradient of hexanes:EtOAc (9:1 to 3:2) to give the title compound (72 mg, 44%). MS (ES, m/z): 407 [M+1]⁺. ¹H NMR (400 MHz, CDCl₃) δ 7.34 (d, J=8.6 Hz, 0.67H), 7.30 (d, J=8.6 Hz, 0.33H), 7.19 (d, J=2.4 Hz, 0.33H), 7.09 (dd, J=8.6, 2.4 Hz, 0.67H), 7.04 (d, J=2.4 Hz, 0.33H), 7.03-6.99 (m, 0.67H), 2.47 (t, J=6.1 Hz, 1.34H), 2.14 (m, 2.68H), 1.79-1.59 (m, 4.02H).

### Example 31

### 1-(4-cyclopropyl-1,2,3,4-tetrahydroquinoxalin-1-yl)-3-(2,5-dichlorophenoxy)-2,2-dimethylpropan-1-one

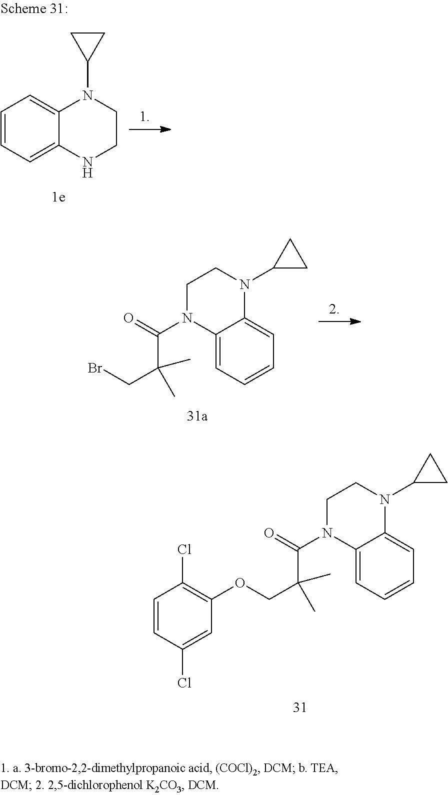 [SMILES: c1ccc2c(c1)NCCN2C1CC1 ; CC(C)(CBr)C(=O)N1CCN(C2CC2)c2ccccc21 ; CC(C)(COc1cc(Cl)ccc1Cl)C(=O)N1CCN(C2CC2)c2ccccc21]

Intermediate 31a: 3-bromo-1-(4-cyclopropyl-1,2,3,4-tetrahydroquinoxalin-1-yl)-2,2-dimethylpropan-1-one. To a stirred 0° C. solution of 3-bromo-2,2-dimethylpropanoic acid (120 mg, 0.660 mmol, 1.16 equiv) in dichloromethane (10 mL) was added oxalyl chloride (2.0 mL) drop-wise. The resulting solution was stirred for 2 h at room temperature then concentrated under reduced pressure to provide a residue of the acid chloride used in the next step without additional purification. The acid chloride residue in dichloromethane (10 mL) was added to a stirred 0° C. solution of 1-cyclopropyl-1,2,3,4-tetrahydroquinoxaline (100 mg, 0.57 mmol, 1.0 equiv) and triethylamine (87 mg, 0.86 mmol, 1.5 equiv) in dichloromethane (10 mL). The resulting solution was stirred for 4 h at room temperature then diluted with H₂O (20 mL) and extracted with dichloromethane (3×20 mL). The combined organic extract was washed with brine (2×20 mL) and dried over anhydrous sodium sulfate then concentrated under reduced pressure. The resulting residue was purified by silica gel chromatography using an eluent of ethyl acetate/petroleum ether (1:5) to provide the product (150 mg, 77%) as pink oil. MS (ES, m/z): 337 [M+H]⁺.

Example 31: 1-(4-cyclopropyl-1,2,3,4-tetrahydroquinoxalin-1-yl)-3-(2,5-dichlorophenoxy)-2,2-dimethylpropan-1-one. A solution of 2,5-dichlorophenol (60 mg, 0.37 mmol, 1.0 equiv), 3-bromo-1-(4-cyclopropyl-1,2,3,4-tetrahydroquinoxalin-1-yl)-2,2-dimethylpropan-1-one (80 mg, 0.24 mmol, 0.64 equiv) and potassium carbonate (64 mg, 0.46 mmol, 1.26 equiv) in N,N-dimethylformamide (4 mL) was stirred for 5 h at 50° C. The resulting solution was diluted with H₂O (10 mL) and extracted with ethyl acetate (3×20 mL). The combined organic extract was washed with brine (1×20 mL) and dried over anhydrous sodium sulfate then concentrated under reduced pressure. The crude product (100 mg) was purified by preparative HPLC with a C₁₈ silica gel stationary phase using a 40 min gradient (H₂O 0.05% TFA:CH₃CN 0.05% TFA 95:5 to 0:100%) and detection by UV at 254 nm to provide TFA salt of the title compound (53 mg, 34%) as a yellow solid. MS (ES, m/z): 419 [M+H]⁺. ¹H-NMR (300 MHz, CD₃OD) δ 7.35 (d, J=8.4 Hz, 1H), 7.20-7.23 (m, 1H), 7.08-7.14 (m, 2H), 6.91-6.95 (m, 1H), 6.67-6.76 (m, 2H), 3.90 (s, 3H), 3.83 (t, J=5.7 Hz, 2H), 3.43 (t, J=6.0 Hz, 2H), 2.31-2.35 (m, 1H), 1.40 (s, 3H), 0.70-0.76 (m, 2H), 0.33-0.38 (m, 2H).

### Example 32

### 1-cyclopropyl-4-[[1-(2,5-dichlorophenoxymethyl)cyclopropyl]carbonyl]-1,2,3,4-tetrahydroquinoxaline

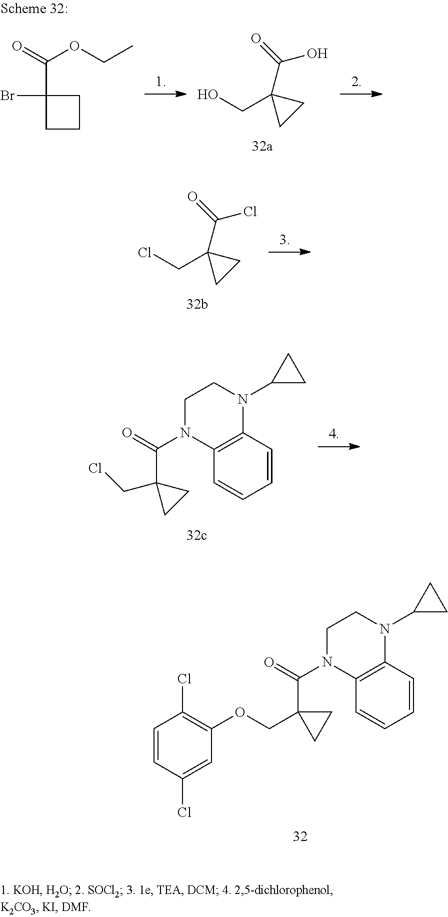

Intermediate 32a: 1-(hydroxymethyl)cyclopropane-1-carboxylic acid. A solution of potassium hydroxide (1.90 g, 33.9 mmol, 2.00 equiv) and ethyl 1-bromocyclobutane-1-carboxylate (3.50 g, 16.9 mmol, 1.00 equiv) in water (60 mL) was stirred overnight at 30° C. The reaction mixture was cooled on ice and the pH value of the solution was adjusted to 1 with concentrated HCl, then concentrated under reduced pressure. The resulting residue was dissolved in methanol (50 mL), solids were removed by filtration, and the filtrate was concentrated under reduced pressure. The resulting residue was applied onto a silica gel column and eluted with a dichloromethane/methanol mobile phase gradient (100:1 to 20:1) to provide 1.8 g (92%) of the product as a white solid.

Intermediate 32b: 1-(chloromethyl)cyclopropane-1-carbonyl chloride. 1-(hydroxymethyl)cyclopropane-1-carboxylic acid (650 mg, 5.60 mmol, 1.00 equiv) was dissolved in thionyl chloride (8 mL) and stirred for 5 h at 80° C. in an oil bath. The resulting reaction mixture was concentrated under reduced pressure to provide 680 mg (79%) of the product as a light yellow oil.

Intermediate 32c: 1-[[1-(chloromethyl)cyclopropyl]carbonyl]-4-cyclopropyl-1,2,3,4-tetrahydroquinoxaline. To a stirred solution of 1-cyclopropyl-1,2,3,4-tetrahydroquinoxaline (850 mg, 4.88 mmol, 1.10 equiv) and triethylamine (900 mg, 8.89 mmol, 2.00 equiv) in dichloromethane (10 mL) was added dropwise a solution of 1-(chloromethyl)cyclopropane-1-carbonyl chloride (680 mg, 4.44 mmol, 1.00 equiv) in dichloromethane (2 mL). The resulting reaction mixture was stirred overnight at room temperature and then concentrated under reduced pressure. The resulting residue was applied to a silica gel column and eluted with a mobile phase gradient of ethyl acetate/petroleum ether (1:15-1:1) to provide 500 mg (39%) of the product as a light yellow oil.

Example 32: 1-cyclopropyl-4-[[1-(2,5-dichlorophenoxymethyl)cyclopropyl]carbonyl]-1,2,3,4-tetrahydroquinoxaline. A solution of 2,5-dichlorophenol (60 mg, 0.37 mmol, 1.2 equiv), 1-[[1-(chloromethyl)cyclopropyl]carbonyl]-4-cyclopropyl-1,2,3,4-tetrahydroquinoxaline (89.2 mg, 0.31 mmol, 1.0 equiv), potassium carbonate (85.6 mg, 0.62 mmol, 2.0 equiv) and KI (5.0 mg, 0.03 mmol, 0.10 equiv) in N,N-dimethylformamide (3.0 mL) was stirred overnight at 65° C. in an oil bath. Solids were removed from the reaction mixture by filtration and the filtrate concentrated under reduced pressure. The crude product (50 mg) was purified by preparative HPLC with the following conditions: Column, SunFire preparative C18, 19*150 mm 5 nm; Mobile phase gradient, water containing 0.05% TFA:CH₃CN (30:70 to 15:85 over 10 min then to 100% over 1 min); Detector, Waters 2545 UV detector at 254 and 220 nm to provide 12.4 mg (10%) of the title compound trifluoroacetate salt as a white solid. MS (ES, m/z): 417 [M+H]⁺; ¹H-NMR (300 MHz, CD₃OD) δ 7.31-7.41 (m, 2H), 7.06-7.14 (m, 2H), 6.90-6.94 (m, 1H), 6.68-6.73 (m, 1H), 6.60-6.61 (m, 1H), 3.86-3.90 (m, 2H), 3.68 (s, 2H), 3.32-3.39 (m, 2H), 2.20-2.29 (m, 1H), 1.36-1.40 (m, 2H), 0.96-0.99 (m, 2H), 0.63-0.69 (m, 2H), 0.16-0.21 (m, 2H).

### Example 33

### 3-[2,5-dichloro-4-([1-[(4-cyclopropyl-1,2,3,4-tetrahydroquinoxalin-1-yl)carbonyl]cyclopropyl]methoxy)phenyl]propanoic acid

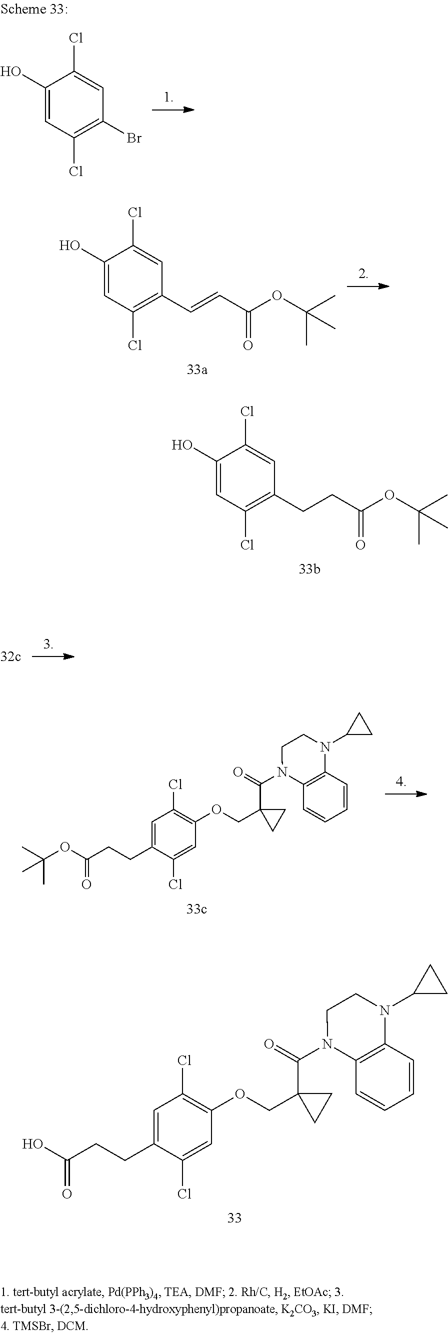

Intermediate 33a: tert-butyl(2E)-3-(2,5-dichloro-4-hydroxyphenyl)prop-2-enoate. A stirred solution of 4-bromo-2,5-dichlorophenol (10.0 g, 41.3 mmol, 1.00 equiv), tert-butyl prop-2-enoate (5.00 g, 39.0 mmol, 1.00 equiv), triethylamine (8.30 g, 82.0 mmol, 2.00 equiv) and Pd(PPh₃)₄ (2.00 g, 1.73 mmol, 0.05 equiv) was purged and maintained under an inert atmosphere of nitrogen then heated overnight at 110° C. in an oil bath. The resulting reaction mixture was diluted with 200 mL of dichloromethane washed with brine (2×100 mL) and the combined organic phase concentrated under reduced pressure. The resulting residue was applied to a silica gel column and eluted with ethyl acetate/petroleum ether (1:10) to provide 8 g (67%) of the product as a yellow solid.

Intermediate 33b: tert-butyl 3-(2,5-dichloro-4-hydroxyphenyl)propanoate. To a stirred solution of tert-butyl(2E)-3-(2,5-dichloro-4-hydroxyphenyl)prop-2-enoate (8.0 g, 27.7 mmol, 1.00 equiv) and Rhodium on Carbon (8.0 g) in ethyl acetate (50 mL) was introduced hydrogen gas. The resulting reaction mixture was stirred overnight at 25° C. solids were removed by Titration and the Titrate concentrated under reduced pressure. The residue was applied onto a silica gel column with and eluted with a mobile phase of ethyl acetate/petroleum ether (1:10) to provide 7 g (87%) of the product as a white solid. MS (ES, m/z): 289 [M−H]⁻; ¹H-NMR (300 MHz, CDCl₃) δ 7.20 (s, 1H), 5.69 (d, J=18 Hz, 1H), 2.92 (m, 2H), 2.52 (m, 2H), 1.43 (s, 9H).

Intermediate 33c: tert-butyl 3-[2,5-dichloro-4-([1-[(4-cyclopropyl-1,2,3,4-tetrahydroquinoxalin-1-yl)carbonyl]cyclopropyl]methoxy)phenyl]propanoate. A solution of tert-butyl 3-(2,5-dichloro-4-hydroxyphenyl)propanoate (539 mg, 1.85 mmol, 1.20 equiv), 1-[[1-(chloromethyl)cyclopropyl]carbonyl]-4-cyclopropyl-1,2,3,4-tetrahydroquinoxaline (450 mg, 1.55 mmol, 1.00 equiv), potassium carbonate (426 mg, 3.08 mmol, 2.00 equiv), KI (24.9 mg, 0.15 mmol, 0.10 equiv) in N,N-dimethylformamide (8 mL) was stirred overnight at 70° C. in an oil bath. The reaction mixture was diluted with water (20 mL) and extracted with ethyl acetate (4×20 mL) and the organic layers combined. The combined organic phase was washed with brine (2×20 mL), dried over anhydrous sodium sulfate and concentrated under reduced pressure. The residue was applied to a silica gel column and eluted with a mobile phase of ethyl acetate/petroleum ether (1:20-1:1) to provide 710 mg (84%) of the product as a yellow solid.

Example 33: 3-[2,5-dichloro-4-([1-[(4-cyclopropyl-1,2,3,4-tetrahydro-quinoxalin-1-yl)carbonyl]cyclopropyl]methoxy)phenyl]propanoic acid. To a stirred 0° C. solution of tert-butyl 3-[2,5-dichloro-4-([1-[(4-cyclopropyl-1,2,3,4-tetrahydroquinoxalin-1-yl)carbonyl]cyclopropyl]methoxy)phenyl]propanoate (120 mg, 0.22 mmol, 1.00 equiv) in dichloromethane (5.0 mL) was added TMSBr (4.0 mL) dropwise. The resulting reaction mixture was allowed to warm to room temperature, stirred for 1.5 h, then was quenched by the addition of dichloromethane/methanol (10:1). The resulting mixture was concentrated under reduced pressure, the crude residue (100 mg) was purified by Preparative HPLC under the following conditions: Column, SunFire preparative C18, 19*150 mm 5 μm; Mobile phase gradient, water containing 0.05% TFA:CH₃CN (26:74 to 9:91 over 6 min then to 100% over 1 min); Detector, Waters 2545 UV detector at 254 and 220 nm. This resulted in 13.5 mg (13%) of the title compound trifluoroacetate salt as a white solid. MS (ES, m/z): 489 [M+H]⁺; ¹H-NMR (300 MHz, CD₃OD) δ 7.35-7.39 (m, 2H), 7.04-7.14 (m, 2H), 6.67-6.73 (m, 1H), 6.58 (s, 1H), 3.85-3.89 (m, 2H), 3.67 (s, 2H), 3.36-3.40 (m, 2H), 2.90-2.95 (m, 2H), 2.54-2.59 (m, 2H), 2.19-2.23 (m, 1H), 1.37-1.40 (m, 2H), 0.94-0.98 (m, 2H), 0.63-0.69 (m, 2H), 0.11-0.16 (m, 2H).

### Example 34

### 3-[2,5-dichloro-4-([1-[(4-cyclopropyl-1,2,3,4-tetrahydroquinoxalin-1-yl)carbonyl]cyclopropyl]methoxy)phenyl]-N-methyl-N-[(2S,3R,4R,5R)-2,3,4,5,6-pentahydroxyhexyl]propanamide

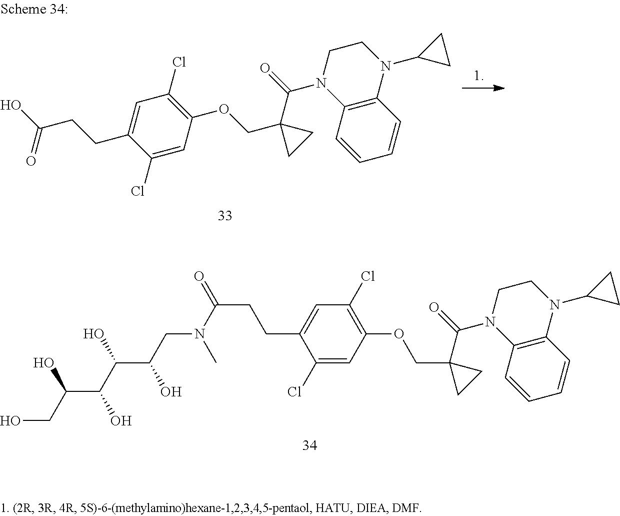 [SMILES: O=C(O)CCc1cc(Cl)c(OCC2(C(=O)N3CCN(C4CC4)c4ccccc43)CC2)cc1Cl ; CN(C[C@H](O)[C@@H](O)[C@H](O)[C@H](O)CO)C(=O)CCc1cc(Cl)c(OCC2(C(=O)N3CCN(C4CC4)c4ccccc43)CC2)cc1Cl]

Example 34: 1-Cyclopropyl-4-([1-[(isoquinolin-5-yloxy)methyl]cyclopropyl]carbonyl)-1,2,3,4-tetrahydroquinoxaline. A solution of 3-[2,5-dichloro-4-([1-[(4-cyclopropyl-1,2,3,4-tetrahydroquinoxalin-1-yl)carbonyl]cyclopropyl]methoxy)phenyl]-propanoic acid (200 mg, 0.41 mmol, 1.0 equiv), HATU (232 mg, 0.61 mmol, 1.5 equiv), DIEA (78.8 mg, 0.61 mmol, 1.50 equiv) and (2R,3R,4R,5S)-6-(methylamino)hexane-1,2,3,4,5-pentol (119.1 mg, 0.61 mmol, 1.50 equiv) in N,N-dimethylformamide (10 mL) was stirred overnight at room temperature. The resulting reaction mixture was diluted with brine (30 mL) and extracted with ethyl acetate (4×30 mL) and the organic layers combined. The combined organic phase was washed with brine (20 mL), dried over sodium sulfate and concentrated under reduced pressure. The crude product (200 mg) was purified by preparative HPLC under the following conditions: Column, SunFire preparative C18, 19*150 mm 5 nm; Mobile phase gradient, water containing 0.05% TFA: CH₃CN (56:44 to 38:62 over 6 min then to 100% over 1 min); Detector, Waters 2545 UV detector at 254 and 220 nm to provide 55.3 mg (20%) of the title compound trifluoroacetate salt as a white solid. MS (ES, m/z): 666 [M+H]⁺; ¹H-NMR (300 MHz, CD₃OD) δ 7.36-7.40 (m, 2H), 7.06-7.11 (m, 2H), 6.68-6.70 (m, 1H), 6.60-6.61 (m, 1H), 3.60-4.00 (m, 11H), 3.31-3.38 (m, 3H), 3.01-3.31 (m, 2H), 2.87-2.97 (m, 4H), 2.63-2.81 (m, 2H), 2.24 (s, 1H), 1.37 (m, 2H), 0.95-0.99 (m, 2H), 0.64-0.69 (m, 2H), 0.16 (m, 2H).

### Example 35

### ((S)-1-(2,5-dichlorobenzyl)pyrrolidin-2-yl)(4-methyl-3,4-dihydroquinolin-1(2H)-yl)methanone

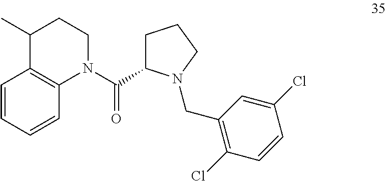 [SMILES: CC1CCN(C(=O)[C@@H]2CCCN2Cc2cc(Cl)ccc2Cl)c2ccccc21]

Example 35: ((S)-1-(2,5-dichlorobenzyl)pyrrolidin-2-yl)(4-methyl-3,4-dihydroquinolin-1(2H)-yl)methanone. 35 was synthesized in an analogous fashion to Example 10, using 4-methyl-1,2,3,4-tetrahydroquinoline in place of 1,2,3,4-tetrahydroquinoline. Isolated as the TFA salt. MS (ES, m/z): 403 [M+H]⁺.

### Example 36

### 1-(2,5-dichlorobenzyloxy)-N-(2-methoxyphenyl)-N-methylcyclopropanecarboxamide

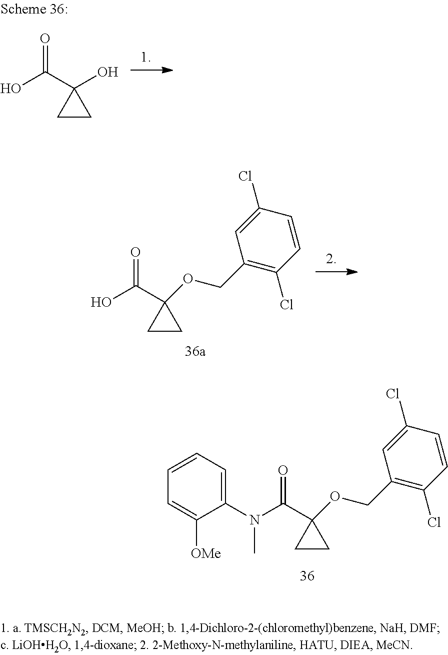 [SMILES: O=C(O)C1(O)CC1 ; O=C(O)C1(OCc2cc(Cl)ccc2Cl)CC1 ; COc1ccccc1N(C)C(=O)C1(OCc2cc(Cl)ccc2Cl)CC1]

Intermediate 36a: 1-(2,5-dichlorobenzyloxy)cyclopropanecarboxylic acid. 1-hydroxycyclopropanecarboxylic acid (204 mg, 2.00 mmol) was dissolved in DCM (2.5 mL) and MeOH (0.5 mL). To the stirring solution was added 2.0 M TMSCH₂N₂ in Et₂O (1.1 mL, 2.2 mmol) dropwise, the resulting solution stirred for 10 min at room temperature. The solvent was removed under reduced pressure and the resulting residue dissolved in DMF (1 mL), followed by addition of 60% NaH in mineral oil dispersion (120 mg) and the resulting suspension stirred for 5 min at room temperature. 1,4-Dichloro-2-(chloromethyl)benzene (583 mg, 3.00 mmol) was added and the suspension stirred at room temperature for 16 h. The suspension was then quenched with 5% aq. HCl and extracted with EtOAc. The organic layer was washed with H₂O and brine, then dried over Na₂SO₄ and the solvent removed under reduced pressure. The crude residue was purified by flash column chromatography using a gradient of hexanes:EtOAc (100:0 to 80:20). The resulting oil was dissolved in H₂O (2 mL) and 1,4-dioxane (4 mL), then LiOH.H₂O (133 mg, 3.18 mmol) added and the solution stirred for 1 h at room temperature, then 1 h at 50° C. The solution was concentrated under reduced pressure, then diluted with 5% aq. HCl and extracted with EtOAc. The organic layer was then washed with brine and dried over Na₂SO₄, then the solvent removed under reduced pressure to give 36a (99 mg, 19%) as a white solid. ¹H NMR (400 MHz, CDCl₃) 7.52 (d, J=2.5 Hz, 1H), 7.29-7.27 (m, 1H), 7.22-7.19 (m, 1H), 4.77 (s, 3H), 1.53-1.48 (m, 2H), 1.41-1.36 (m, 2H).

Example 36: 1-(2,5-dichlorobenzyloxy)-N-(2-methoxyphenyl)-N-methylcyclopropanecarboxamide. Intermediate 36a (24 mg, 0.092 mmol), 2-methoxy-N-methylaniline (16 mg, 0.11 mmol), HATU (38 mg, 0.10 mmol), and DIEA (64 μL, 0.37 mmol) were all combined in MeCN (1 mL) and stirred for 24 h at room temperature. The solution was then purified by preparative HPLC with a C18 silica gel stationary phase using a gradient of H₂O 0.05% TFA: CH3CN 0.05% TFA (70:30 to 5:95) and detection by UV at 254 nm to give the title compound (18 mg, 52%) as a white solid. MS (ES, m/z): 380 [M+1]⁺. ¹H-NMR (400 MHz, CDCl₃) δ 7.30-7.22 (m, 1H), 7.22-7.13 (m, 2H), 7.09 (d, J=8.3 Hz, 1H), 6.88 (t, J=7.5 Hz, 1H), 6.68 (d, J=8.5 Hz, 1H), 6.40 (s, 1H), 4.49 (d, J=13.6 Hz, 1H), 4.31 (d, J=14.0 Hz, 1H), 3.43 (s, 3H), 3.21 (s, 3H), 1.58-1.46 (m, 1H), 1.36-1.24 (m, 2H), 1.09-0.96 (m, 1H), 0.92-0.81 (m, 1H).

### Example 37

### 1-(2,5-dichlorobenzyloxy)-N-(3-methoxypyridin-2-yl)-N-methylcyclopropanecarboxamide

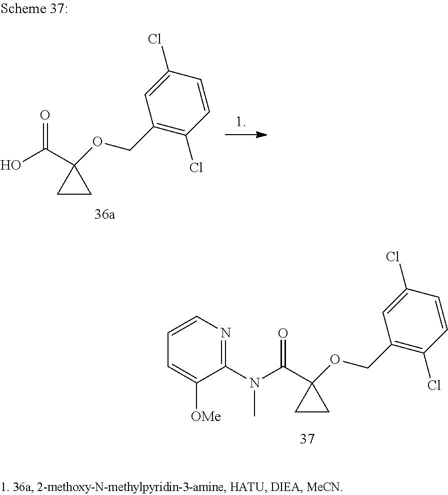 [SMILES: O=C(O)C1(OCc2cc(Cl)ccc2Cl)CC1 ; COc1cccnc1N(C)C(=O)C1(OCc2cc(Cl)ccc2Cl)CC1]

Example 37: 1-(2,5-dichlorobenzyloxy)-N-(3-methoxypyridin-2-yl)-N-methylcyclopropanecarboxamide. The title compound was prepared in the same manner as Example 36, using 2-methoxy-N-methylpyridin-3-amine in place of 2-methoxy-N-methylaniline to give 37 (8%) as the TFA salt. MS (ES, m/z): 381 [M+1]⁻¹. ¹H-NMR (400 MHz, CDCl₃) δ 7.94 (dd, J=4.9, 1.4 Hz, 1H), 7.24 (dd, J=8.2, 4.9 Hz, 1H), 7.18 (d, J=8.5 Hz, 1H), 7.10 (td, J=8.8, 2.0 Hz, 2H), 6.39 (s, 1H), 4.40 (s, 2H), 3.64 (s, 3H), 3.30 (s, 3H), 1.52 (s, 2H), 1.02 (s, 2H).

### Example 38

### 1-(2,5-dichlorobenzylamino)-N-(2-methoxyphenyl)-N-methylcyclopropanecarboxamide

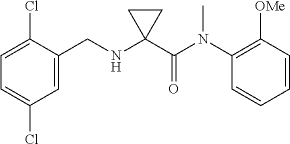 [SMILES: COc1ccccc1N(C)C(=O)C1(NCc2cc(Cl)ccc2Cl)CC1]

Example 38: 1-(2,5-dichlorobenzylamino)-N-(2-methoxyphenyl)-N-methylcyclopropanecarboxamide. The title compound was prepared in the same manner as 27, using 2-methoxy-N-methylaniline in place of 1e. Isolated as the TFA salt. MS (ES, m/z): 378 [M+1]⁺. ¹H-NMR (400 MHz, CDCl₃) δ 7.44-7.40 (m, 1H), 7.39 (d, J=2.2 Hz, 1H), 7.37-7.33 (m, 1H), 7.31-7.27 (m, 1H), 7.14-7.11 (m, 1H), 7.06-6.99 (m, 2H), 4.23 (s, 2H), 3.83 (s, 3H), 3.21 (s, 3H), 1.41 (s, 2H), 1.03 (s, 1H), 0.97-0.85 (m, 1H).

### Example 39

### (S)—N-(2-(cyclopropylmethoxy)phenyl)-1-(2,5-dichlorobenzyl)-N-methylpyrrolidine-2-carboxamide

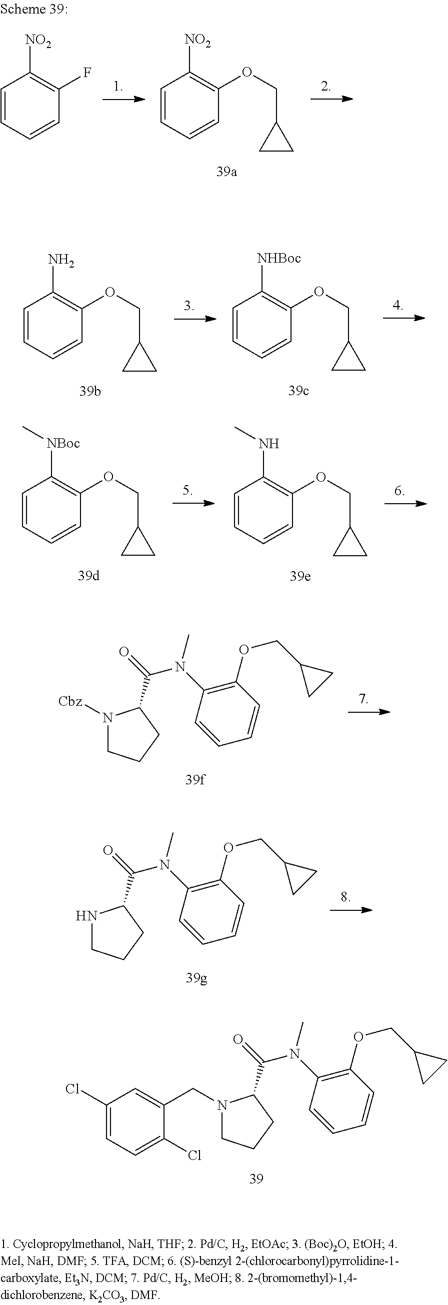

Intermediate 39a: 1-(cyclopropylmethoxy)-2-nitrobenzene. Cyclopropylmethanol (2.55 g, 35.4 mmol) was dissolved in tetrahydrofuran (50 mL). NaH 60% dispersion in mineral oil (1.70 g) was added t to the stirring solution in several batches at 0° C. and the mixture was stirred for 1 h. 1-Fluoro-2-nitrobenzene (5.00 g, 35.4 mmol) was then added and the resulting mixture stirred for 2 h at 80° C. The reaction was then quenched by the addition of 30 mL of water and then extracted thrice with ethyl acetate. The organic layers were combined and dried over anhydrous Na₂SO₄ and concentrated under reduced pressure to give 39a (6.5 g, 95%) as a brown oil. MS (ES, m/z): 193 [M]⁺.

Intermediate 39b: 2-(cyclopropylmethoxy)aniline. 39a (6.50 g, 33.6 mmol) and palladium on carbon (6.5 g) was dissolved in ethyl acetate (50 mL). The suspension was stirred overnight at room temperature under an atmosphere of H₂. The suspension was filtered and the filtrate concentrated under reduced pressure to give 39b (5.68 g), as a red oil, which was used without further purification. MS (ES, m/z): 164 [M+H]⁺.

Intermediate 39c: tert-butyl N-[2-(cyclopropylmethoxy)phenyl]carbamate. 39b (5.68 g, 34.8 mmol) was dissolved in ethanol (50 mL), followed by addition of di-tert-butyl dicarbonate (9.12 g, 41.8 mmol) in several batches. The resulting solution was stirred for 3 h at room temperature, then the solvent removed under reduced pressure to give 39c (9 g, 98%) as a red oil.

Intermediate 39d: tert-butyl N-[2-(cyclopropylmethoxy)phenyl]-N-methylcarbamate. 39d (9.00 g, 34.2 mmol) was dissolved in N,N-dimethylformamide (150 mL), followed by the addition of NaH 60% dispersion in mineral oil (2.1 g) in several batches at 0° C. The mixture was stirred for 1 h at room temperature, then iodomethane (9.70 g, 68.3 mmol) added and the resulting solution stirred overnight at room temperature. The solvent was then removed under reduced pressure and the resulting residue dissolved in ethyl acetate and washed with H₂O and brine. The organic layer was then dried over anhydrous sodium sulfate and concentrated under reduced pressure and the residue purified by normal-phase flash column chromatography, using ethyl acetate:petroleum ether (1:50) as eluent to give 39d 8.0 g (84%) as a red oil.

Intermediate 39e: 2-(cyclopropylmethoxy)-N-methylaniline. 39d (2.00 g, 7.21 mmol) was dissolved in dichloromethane (3 mL) and trifluoroacetic acid (3 mL). The was stirred for 1 h at room temperature, then the solvent removed under reduced pressure. The resulting residue was dissolved in DCM and washed with saturated aqueous NaHCO₃. The aqueous layer was then extracted thrice with DCM and the organic layers combined and dried over anhydrous sodium sulfate solvent removed under reduced pressure to give 39e (870 mg, 68%) as a red oil. MS (ES, m/z): 178 [M+H]⁺.

Intermediate 39f: Benzyl(2S)-2-(carbonochloridoyl)pyrrolidine-1-carboxylate (260 mg, 0.970 mmol) and triethylamine (202 mg, 2.00 mmol) were dissolved in DCM (4 mL). To this solution was added 39e (177 mg, 1.00 mmol) and the resulting solution stirred for 3 h at room temperature. The reaction was then quenched by the addition of 10 mL of water and the mixture extracted thrice with DCM and the organic layers combined, dried over anhydrous sodium sulfate, and concentrated under reduced pressure to give 39f (400 mg, 99%) as a yellow oil. MS (ES, m/z): 409 [M+H]⁺.

Intermediate 39g: (2 S)—N-[2-(cyclopropylmethoxy)phenyl]-N-methylpyrrolidine-2-carboxamide. 39f (380 mg, 0.93 mmol) and palladium on carbone (400 mg) was added to methanol (5 mL). The resulting suspension was stirred for 2 h at room temperature under an atmosphere of H₂. The suspension was filtered and the filtrate concentrated under reduced pressure to give 39g (200 mg, 78%) as a colorless oil. MS (ES, m/z): 275 [M+H]⁺.

Example 39: (S)—N-(2-(cyclopropylmethoxy)phenyl)-1-(2,5-dichlorobenzyl)-N-methylpyrrolidine-2-carboxamide. 39g (200 mg, 0.73 mmol), 2-(bromomethyl)-1,4-dichlorobenzene (176 mg, 0.73 mmol), and potassium carbonate (203 mg, 1.47 mmol) were dissolved in N,N-dimethylformamide (4 mL). The resulting solution was stirred overnight at room temperature, then diluted with 20 mL of water and extracted thrice with ethyl acetate. The organic layers were combined, dried over anhydrous sodium sulfate, and the solvent removed under reduced pressure. The crude residue was purified by normal-phase flash column chromatography, using ethyl acetate:petroleum ether (1:8) to give Example 39 (100 mg, 32%) as a yellow oil. MS (ES, m/z): 433 [M+H]⁺. ¹H-NMR (300 MHz, CD₃OD, ppm): 7.46 (d, J=2.4 Hz, 0.6H), 7.34 (d, J=2.7 Hz, 0.4H), 7.15-6.97 (m, 3.6H), 6.88-6.78 (m, 1.6H), 6.67-6.61 (m, 0.8H), 3.71-3.59 (m, 3H), 3.41 (d, J=14.6 Hz, 0.4H), 3.22 (d, J=14.6 Hz, 0.6H), 3.02-2.96 (m, 2.2H), 2.93-2.80 (m, 2H), 2.72 (m, 0.6H), 2.13 (m, 0.4H), 1.87 (m, 0.6H), 1.73-1.52 (m, 2.6H), 1.52-1.35 (m, 1.4H), 1.10-0.84 (1.2H), 0.42-0.32 (m, 0.8H), 0.32-0.18 (m, 1.2H), 0.14:0.06.

### Example 40

### N-(2-(cyclopropylmethoxy)phenyl)-1-(2,5-dichlorobenzyloxy)-N-methylcyclopropanecarboxamide

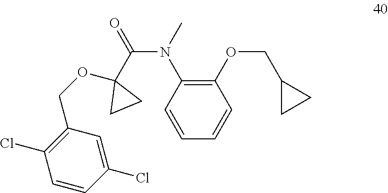 [SMILES: CN(C(=O)C1(OCc2cc(Cl)ccc2Cl)CC1)c1ccccc1OCC1CC1]

Example 40: N-(2-(cyclopropylmethoxy)phenyl)-1-(2,5-dichlorobenzyloxy)-N-methylcyclopropanecarboxamide. 40 was synthesized in an analogous fashion to Example 36, using 39e in place of 2-Methoxy-N-methylaniline. MS (ES, m/z): 420 [M+H]⁺. ¹H-NMR (300 MHz, CD₃OD) δ 7.30-7.18 (m, 4H), 6.92 (t, J=7.5 Hz, 1H), 6.75 (d, J=7.8 Hz, 1H), 6.43 (s, 1H), 4.44 (dd, J=13.8, 26.7 Hz, 2H), 3.56-3.50 (m, 1H), 3.32-3.22 (m, 4H), 1.51-1.50 (m, 1H), 1.49-1.46 (m, 1H), 1.32-1.04 (m, 2H), 0.95-0.89 (m, 1H), 0.55-0.52 (m, 2H), 0.24-0.16 (m, 2H).

### Example 41

### 1-cyclopropyl-4-([1-[(2,5-dichlorophenyl)methoxy]cyclobutyl]carbonyl)-1,2,3,4-tetrahydroquinoxaline

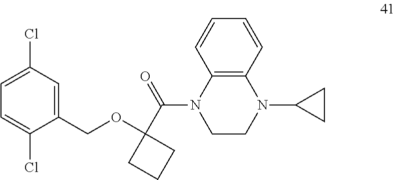 [SMILES: O=C(N1CCN(C2CC2)c2ccccc21)C1(OCc2cc(Cl)ccc2Cl)CCC1]

Example 41: 1-cyclopropyl-4-([1-[(2,5-dichlorophenyl)methoxy]-cyclobutyl]carbonyl)-1,2,3,4-tetrahydroquinoxaline. 41 was synthesized in an analogous fashion to Example 8, using cyclobutanone in place of cyclopentanone. Isolated as the TFA salt. MS (ES, m/z): 431 [M+H]⁺. ¹H-NMR (400 MHz, CD₃OD) δ 6.56-7.55 (m, 7H), 4.02-4.46 (m, 2H), 3.86 (t, J=5.6 Hz, 2H), 3.36 (t, J=5.6 Hz, 2H), 2.75-2.81 (m, 2H), 2.38 (m, 3H), 1.82-2.19 (m, 2H), 0.04-0.92 (m, 4H).

### Example 42

### (1-(5-chloro-2-(trifluoromethyl)benzyloxy)cyclopropyl)(4-cyclopropyl-3,4-dihydroquinoxalin-1(2H)-yl)methanone

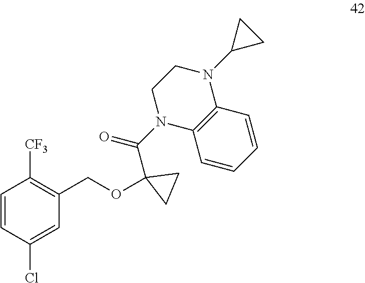 [SMILES: O=C(N1CCN(C2CC2)c2ccccc21)C1(OCc2cc(Cl)ccc2C(F)(F)F)CC1]

Example 42: (1-(5-chloro-2-(trifluoromethyl)benzyloxy)cyclopropyl)(4-cyclopropyl-3,4-dihydroquinoxalin-1(2H)-yl)methanone. Example 42 was synthesized in an analogous fashion to Example 9, using 2-(bromomethyl)-4-chloro-1-(trifluoromethyl)benzene in place of 2-(bromomethyl)-1,4-dichlorobenzene. Isolated as the TFA salt. MS (ES, m/z): 451 [M+H]⁺. ¹H-NMR (300 MHz, CD₃OD) δ 7.53 (d, J=9 Hz, 1H), 7.32 (m, 2H), 7.07 (m, 1H), 6.95 (d, J=9 Hz, 1H), 6.75 (m, 1H), 6.59 (s, 1H), 4.42 (s, 2H), 3.85 (t, J=6 Hz, 2H), 3.33 (t, J=6 Hz, 2H), 2.24-2.17 (m, 1H), 1.45 (m, 2H), 1.16-1.12 (m, 2H), 0.61-0.56 (m, 2H), 0.01 (m, 2H).

### Example 43

### 1-[(1-[[2-chloro-5-(trifluoromethyl)phenyl]methoxy]cyclopropyl)carbonyl]-4-cyclopropyl-1,2,3,4-tetrahydroquinoxaline

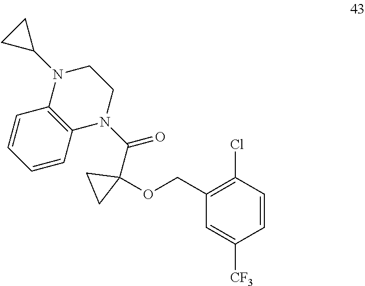 [SMILES: O=C(N1CCN(C2CC2)c2ccccc21)C1(OCc2cc(C(F)(F)F)ccc2Cl)CC1]

Example 43: (1-(5-chloro-2-(trifluoromethyl)benzyloxy)cyclopropyl)(4-cyclopropyl-3,4-dihydroquinoxalin-1(2H)-yl)methanone. 43 was synthesized in an analogous fashion to Example 9, using 2-(bromomethyl)-1-chloro-4-(trifluoromethyl)benzene in place of 2-(bromomethyl)-1,4-dichlorobenzene. Isolated as the TFA salt. MS (ES, m/z): 451 [M+H]⁺. ¹H-NMR (400 MHz, CD₃OD) δ 7.64 (s, 2H), 7.33 (d, J=4 Hz, 1H), 7.01-6.92 (m, 3H), 6.72 (t, J=8 Hz, 1H), 4.45 (s, 2H), 3.93 (t, J=4 Hz, 2H), 3.41 (t, J=4 Hz, 2H), 2.27-2.25 (m, 1H), 1.54-1.48 (m, 2H), 1.31-1.21 (m, 2H), 0.65 (m, 2H).

### Example 44

### (4-cyclopropyl-3,4-dihydroquinoxalin-1(2H)-yl)(1-(2,6-dichlorobenzyloxyl)cyclopropyl)methanone

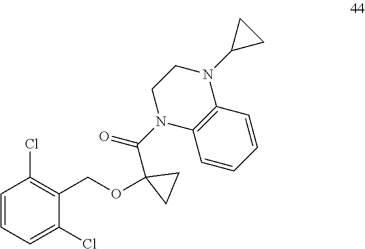 [SMILES: O=C(N1CCN(C2CC2)c2ccccc21)C1(OCc2c(Cl)cccc2Cl)CC1]

Example 44: (4-cyclopropyl-3,4-dihydroquinoxalin-1(2H)-yl)(1-(2,6-dichlorobenzyloxyl)cyclopropyl)methanone. Example 44 was synthesized in an analogous fashion to Example 9, using 2-(bromomethyl)-1,3-dichlorobenzene in place of 2-(bromomethyl)-1,4-dichlorobenzene. Isolated as the TFA salt. MS (ES, m/z): 417 [M+H]⁺. ¹H-NMR (300 MHz, CD₃OD) δ 7.41-7.32 (m, 3H), 7.29 (m, 1H), 7.28-7.22 (m, 1H), 7.05-6.99 (m, 1H), 6.68-6.62 (m, 1H), 4.90 (s, 2H), 4.10 (t, J=6 Hz, 2H), 3.41 (t, J=6 Hz, 2H), 2.43-2.38 (m, 1H), 1.20 (s, 4H), 0.86-0.81 (m, 2H), 0.65-0.60 (m, 2H).

### Example 45

### 3-((1-(4-cyclopropyl-1,2,3,4-tetrahydroquinoxaline-1-carbonyl)cyclopropoxy)methyl)benzonitrile

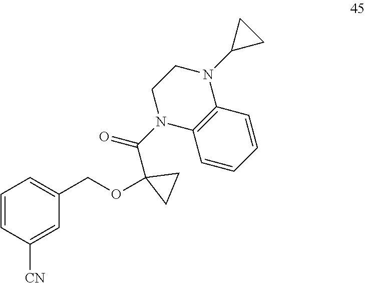 [SMILES: N#Cc1cccc(COC2(C(=O)N3CCN(C4CC4)c4ccccc43)CC2)c1]

Example 45: 3-((1-(4-cyclopropyl-1,2,3,4-tetrahydroquinoxaline-1-carbonyl)cyclopropoxy)methyl)benzonitrile. Example 45 was synthesized in an analogous fashion to Example 9, using 3-(bromomethyl)benzonitrile in place of 2-(bromomethyl)-1,4-dichlorobenzene. Isolated as the TFA salt. MS (ES, m/z): 374 [M+H]⁺. ¹H-NMR (400 MHz, CD₃OD) δ 7.55 (d, J=8 Hz, 1H), 7.37 (t, J=8 Hz, 2H), 7.23 (s, 1H), 7.18-7.10 (m, 2H), 6.92 (m, 1H), 6.77-6.73 (m, 1H), 4.41 (s, 2H), 3.94 (t, J=4 Hz, 2H), 3.40 (t, J=8 Hz, 2H), 2.34-2.29 (m, 1H), 1.44 (m, 2H), 1.19-1.17 (m, 2H), 0.73-0.65 (m, 2H).

### Example 46

### (S)-(1-(5-chloro-2-(trifluoromethyl)benzyl)pyrrolidin-2-yl)(4-cyclopropyl-3,4-dihydroquinoxaline-1(2H)-yl)methanone

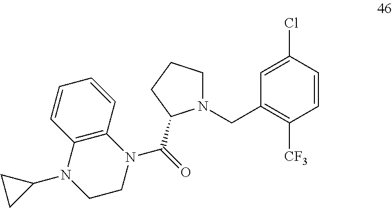 [SMILES: O=C([C@@H]1CCCN1Cc1cc(Cl)ccc1C(F)(F)F)N1CCN(C2CC2)c2ccccc21]

Example 46: (S)-(1-(5-chloro-2-(trifluoromethyl)benzyl)pyrrolidin-2-yl)(4-cyclopropyl-3,4-dihydroquinoxaline-1(2H)-yl)methanone. Example 46 was prepared using the procedure described for the preparation of Example 12 except that 2-(bromomethyl)-4-chloro-1-(trifluoromethyl)benzene was used in place of 2-(bromomethyl)-1,4-dichlorobenzene. Isolated as the bis TFA salt. MS (ES, m/z): 464 [M+H]⁺. ¹H-NMR (300 MHz, CD₃OD) δ 8.01 (s, 1H), 7.69-7.81 (m, 2H), 7.24 (s, 2H), 7.04-7.07 (m, 1H), 6.78-6.81 (m, 1H), 4.56-4.87 (m, 2H), 3.99 (m, 1H), 3.17-3.77 (m, 6H), 2.47 (t, J=4.8 Hz, 1H), 1.83-2.18 (m, 4H), 0.87-0.91 (m, 2H), 0.50-0.66 (m, 2H).

### Example 47

### (S)(4-cyclopropyl-3,4-dihydroquinoxaline-1(2H)-yl)(1-(2,6-dichlorobenzyl)pyrrolidin-2-yl) methanone

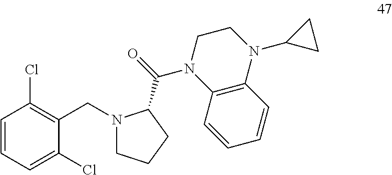 [SMILES: O=C([C@@H]1CCCN1Cc1c(Cl)cccc1Cl)N1CCN(C2CC2)c2ccccc21]

Example 47: (S)(4-cyclopropyl-3,4-dihydroquinoxaline-1(2H)-yl)(1-(2,6-dichlorobenzyl) pyrrolidin-2-yl) methanone. Example 47 was prepared using the procedure described for the preparation of Example 12 except that 2-(bromomethyl)-1,3-dichlorobenzene was used in place of 2-(bromomethyl)-1,4-dichlorobenzene. MS (ES, m/z): 430 [M+H]⁺. ¹H-NMR (300 MHz, CD₃OD) δ 7.50-7.56 (m, 3H), 7.12 (m, 2H), 7.10-7.12 (m, 1H), 6.60-6.81 (m, 1H), 4.98 (m, 1H), 4.78 (m, 1H), 3.33-3.85 (m, 6H), 3.12-3.14 (m, 1H), 1.90-2.51 (m, 5H), 0.88-0.92 (m, 2H), 0.56-0.69 (m, 2H).

### Example 48

### 3-(2,5-dichloro-4-(((S)-2-(4-cyclopropyl-1,2,3,4-tetrahydroquinoxaline-1carbonyl)pyrrolidin-1-yl)methyl)phenyl)-N-methyl-N-((2S,3R,4R,5R)-2,3,4,5,6-pentahydroxyhexyl)propanamide

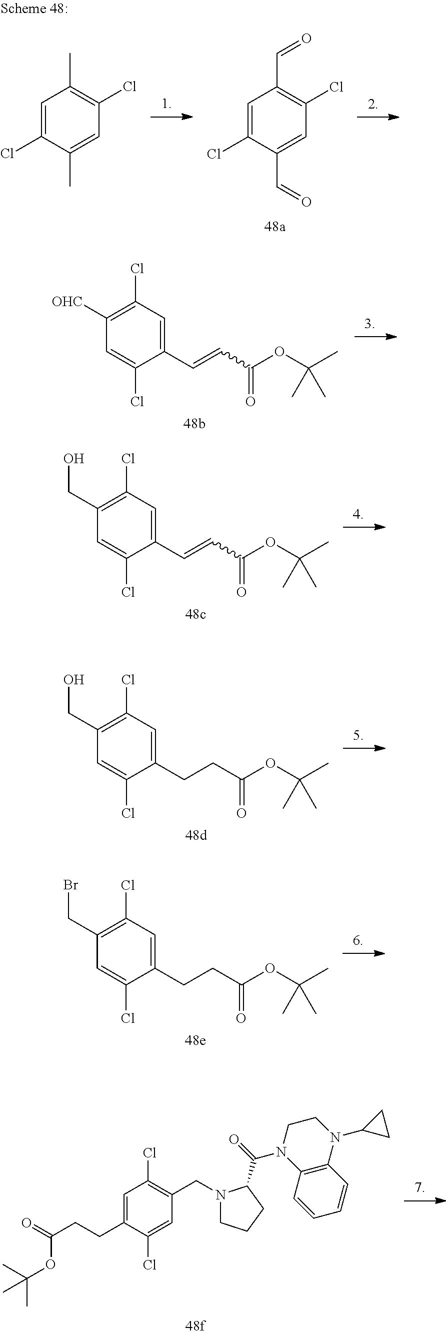

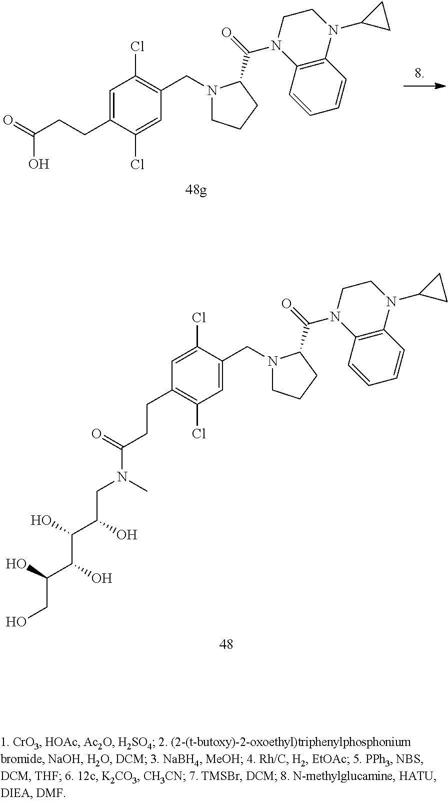 [SMILES: O=C(O)CCc1cc(Cl)c(CN2CCC[C@H]2C(=O)N2CCN(C3CC3)c3ccccc32)cc1Cl ; CN(C[C@H](O)[C@@H](O)[C@H](O)[C@H](O)CO)C(=O)CCc1cc(Cl)c(CN2CCC[C@H]2C(=O)N2CCN(C3CC3)c3ccccc32)cc1Cl]

Intermediate 48a: 2,5-Dichloroterephthalaldehyde. To a mixture of acetic acid (300 g, 5.00 mol, 25.0 equiv), acetic anhydride (600 g, 5.88 mol, 29.4 equiv) and sulfuric acid (90.0 g, 899 mmol, 4.50 equiv, 98%) at 0-10° C. was added 1,4-dichloro-2,5-dimethylbenzene (35.0 g, 200 mmol, 1.00 equiv), and followed by addition of chromium trioxide (60.0 g, 600 mmol, 3.00 equiv) in several batches over 2 h. The resulting solution was stirred for 4 h at room temperature. and then quenched by the addition of 2000 mL of crushed ice. The resulting solution was extracted with 3×1000 mL of ethyl acetate and the organic layers were combined and concentrated under reduced pressure to give a solid. The solid was added to a mixture of ethanol (300 mL), water (300 mL), and sulfuric acid (30 mL), and the mixture was heated to reflux for 3 h and then cooled. The solids were collected by filtration. The solid was purified by column with ethyl acetate/petroleum ether (1:30) to give 12 g (30%) of 2,5-dichloroterephthalaldehyde as a white solid.

Intermediate 48b: tert-Butyl 3-(2,5-dichloro-4-formylphenyl)acrylate. To a solution of 2,5-dichloroterephthalaldehyde (10.0 g, 49.3 mmol, 1.00 equiv) in dichloromethane (200 mL) at 0° C. was added (2-(tert-butoxy)-2-oxoethyl)triphenylphosphanium bromide (15.9 g, 34.8 mmol, 0.70 equiv), and followed by addition of a solution of sodium hydroxide (9.9 g, 0.25 mol, 5.00 equiv) in water (14.5 mL) dropwise with stirring. The resulting solution was stirred for 1 h at 0° C. The resulting mixture was concentratedconcentrated under reduced pressure. The residue was purified by column with ethyl acetate/petroleum ether (1:200˜1:30) to give 9.8 g (66%) of tert-butyl 3-(2,5-dichloro-4-formylphenyl)acrylate as a white solid.

Intermediate 48c: tert-butyl 3-[2,5-dichloro-4-(hydroxymethyl)-phenyl]acrylate. To a solution of tert-butyl 3-(2,5-dichloro-4-formylphenyl)prop-2-enoate (6.9 g, 22.9 mmol, 1.00 equiv) in methanol (100 mL) was added NaBH₄ (1.60 g, 42.3 mmol, 2.00 equiv). The resulting solution was stirred for 1 h at room temperature.

The reaction was then quenched by the addition of 10 mL of water and concentrated under reduced pressure. The resulting mixture was diluted with 100 mL of brine, extracted with ethyl acetate (2×200 mL). The organic layers were combined, dried over sodium sulfate and concentrated under reduced pressure. The residue was purified by column with ethyl acetate/petroleum ether (1:200-1:30 to provide 6.3 g (91%) of tert-butyl 3-[2,5-dichloro-4-(hydroxymethyl)phenyl]acrylate as colorless oil.

Intermediate 48d: tert-butyl 3-[2,5-dichloro-4-(hydroxymethyl)phenyl]-propanoate. To a solution of tert-butyl 3-[2,5-dichloro-4-(hydroxymethyl)phenyl]acrylate (600 mg, 1.00 equiv) in ethyl acetate (20 mL) was added Rh/C (600 mg). The resulting solution was stirred overnight under H₂ at room temperature. The solids were filtered out. The resulting mixture was concentrated under reduced pressure. The residue was applied onto a silica gel column with ethyl acetate/petroleum ether (1:30) to give 500 mg (82%) of tert-butyl 3-[2,5-dichloro-4-(hydroxymethyl)phenyl]propanoate as light brown oil. ¹H NMR (300 MHz, DMSO) δ 7.52 (s, 1H), 7.40 (s, 1H), 5.51 (t, J=5.7 Hz, 1H), 4.51 (d, J=5.7 Hz, 2H), 2.90 (t, J=7.4 Hz, 2H), 2.59-2.51 (m, 2H), 1.37 (s, 9H).

Intermediate 48e: tert-butyl 3-[4-(bromomethyl)-2,5-dichlorophenyl]-propanoate. To a solution of tert-butyl 3-[2,5-dichloro-4-(hydroxymethyl)phenyl]propanoate (800 mg, 2.62 mmol, 1.00 equiv) in dichloromethane/tetrahydrofuran (5/5 mL) at 0° C. were added NBS (1034 mg, 5.81 mmol, 2.00 equiv) and PPh₃ (888 mg, 3.39 mmol, 1.20 equiv). The resulting solution was stirred for 2 h at room temperature. The resulting solution was diluted with ethyl acetate (30 mL), washed with brine (2×20 mL), dried over sodium sulfate, and concentrated under reduced pressure. The residue was applied onto a silica gel column with ethyl acetate/petroleum ether (1:200˜1:20) to give 600 mg (62%) of tert-butyl 3-[4-(bromomethyl)-2,5-dichlorophenyl]propanoate as a white solid.

Intermediate 48f: (S)-tert-butyl 3-(2,5-dichloro-4-((2-(4-cyclopropyl-1,2,3,4-tetrahydroquinoxaline-1-carbonyl)pyrrolidin-1-yl)methyl)phenyl)propanoate. To a solution of tert-butyl 3-[4-(bromomethyl)-2,5-dichlorophenyl]propanoate (100 mg, 0.27 mmol, 1.00 equiv) in CH₃CN (2 mL) were added 12c (110 mg, 0.41 mmol, 1.50 equiv) and potassium carbonate (75 mg, 0.54 mmol, 2.00 equiv). The resulting solution was stirred overnight at room temperature. The resulting mixture was concentrated under reduced pressure. The residue was purified by preparative TLC with ethyl acetate/petroleum ether (1:3) to give 50 mg (33%) of (S)-tert-butyl 3-(2,5-dichloro-4-((2-(4-cyclopropyl-1,2,3,4-tetrahydroquinoxaline-1-carbonyl)pyrrolidin-1-yl)methyl)phenyl)propanoate as light brown oil. MS (ES, m/z): 558 [M+H]⁺.

Intermediate 48g: (S)-3-(2,5-dichloro-4-((2-(4-cyclopropyl-1,2,3,4-tetrahydroquinoxaline-1-carbonyl)pyrrolidin-1-yl)methyl)phenyl)propanoic acid. To a solution of (S)-tert-butyl 3-(2,5-dichloro-4-((2-(4-cyclopropyl-1,2,3,4-tetrahydroquinoxaline-1-carbonyl)pyrrolidin-1-yl)methyl)phenyl)propanoate (50 mg, 0.090 mmol, 1.00 equiv) in dichloromethane (2 mL) was added TMSBr (1 mL). The resulting solution was stirred for 2 h at room temperature. The resulting mixture was concentrated under reduced pressure. The residue was dissolved in 20 mL of ethyl acetate, washed with brine (2×10 mL), and concentrated under reduced pressure to provide 50 mg (crude) of (S)-3-(2,5-dichloro-4-((2-(4-cyclopropyl-1,2,3,4-tetrahydroquinoxaline-1-carbonyl)pyrrolidin-1-yl)methyl)phenyl)propanoic acid as light brown oil

Example 48: 3-(2,5-dichloro-4-(((S)-2-(4-cyclopropyl-1,2,3,4-tetrahydro-quinoxaline-1carbonyl)pyrrolidin-1-yl)methyl)phenyl)-N-methyl-N-((2S,3R,4R,5R)-2,3,4,5,6-pentahydroxyhexyl)propanamide. To a solution of (S)-3-(2,5-dichloro-4-((2-(4-cyclopropyl-1,2,3,4-tetrahydroquinoxaline-1-carbonyl)pyrrolidin-1-yl)methyl)phenyl)-propanoic acid (50 mg, 0.10 mmol, 1.0 equiv) in DMF (2 mL) were added (2R,3R,4R,5S)-6-(methylamino)hexane-1,2,3,4,5-pentol (26 mg, 0.13 mmol, 1.5 equiv), HATU (50 mg, 0.13 mmol, 1.50 equiv), and DIEA (23 mg, 0.18 mmol, 2.0 equiv). The resulting solution was stirred overnight at room temperature. The solids were filtered out. The crude product (50 mg) was purified by Prep-HPLC: Column, SunFire Prep-C18, 19*150 mm Sum; mobile phase gradient, water 0.05% TFA: CH₃CN (38% to 50% CH₃CN over 8 min; detector, Waters 2545 UV detector 254/220 nm) to provide 8.7 mg (13%) of 3-(2,5-dichloro-4-(((S)-2-(4-cyclopropyl-1,2,3,4-tetrahydroquinoxaline-1carbonyl)pyrrolidin-1-yl)methyl)phenyl)-N-methyl-N-((2S,3R,4R,5R)-2,3,4,5,6-pentahydroxyhexyl)propanamide bis TFA salt as a white solid. MS (ES, m/z): 679 [M+H]⁺. ¹H-NMR (300 MHz, CD₃OD) δ 7.72 (dd, J=11.8, 5.2 Hz, 1H), 7.56 (s, 1H), 7.41-7.19 (m, 2H), 7.15-7.01 (m, 1H), 6.86-6.75 (m, 0.6H), 6.72-6.59 (m, 0.4H), 4.53 (dd, J=34.6, 14.2 Hz, 2H), 4.24-4.06 (m, 0.8H), 4.04-3.89 (m, 1.2H), 3.87-3.56 (m, 7H), 3.54-3.36 (m, 3H), 3.25-3.16 (m, 1H), 3.16-2.89 (m, 5H), 2.86-2.64 (m, 2H), 2.63-2.22 (m, 2H), 2.19-1.83 (m, 3H), 1.81-1.56 (m, 1.2H), 1.44-1.23 (m, 0.8H), 1.02-0.80 (m, 2H), 0.75-0.42 (m, 2H).

### Example 49

### (2S)—N-(2-cyclobutoxyphenyl)-1-[(2,5-dichlorophenyl)methyl]-N-methylpyrrolidine-2-carboxamide

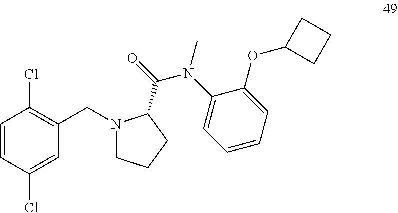 [SMILES: CN(C(=O)[C@@H]1CCCN1Cc1cc(Cl)ccc1Cl)c1ccccc1OC1CCC1]

Example 49: (2S)—N-(2-cyclobutoxyphenyl)-1-[(2,5-dichlorophenyl)methyl]-N-methylpyrrolidine-2-carboxamide. 49 was synthesized in an analogous fashion to Example 39, using cyclobutanol in place of cyclopropylmethanol. MS (ES, m/z): 433 [M+H]⁺. ¹H-NMR (400 MHz, DMSO-d₆) δ 7.63 (s, 1H), 7.26-7.46 (m, 4H), 6.84-7.00 (m, 2H), 4.70-4.72 (m, 1H), 3.53-3.80 (m, 2H), 3.16-3.32 (m, 1H), 3.01-3.16 (m, 4H), 2.37-2.51 (m, 3H), 1.60-1.82 (m, 8H).

### Example 50

### (4-cyclopropyl-3,4-dihydroquinoxalin-1(2H)-yl)(3-(2,5-dichlorobenzyloxyl)azetidin-3-yl)methanone

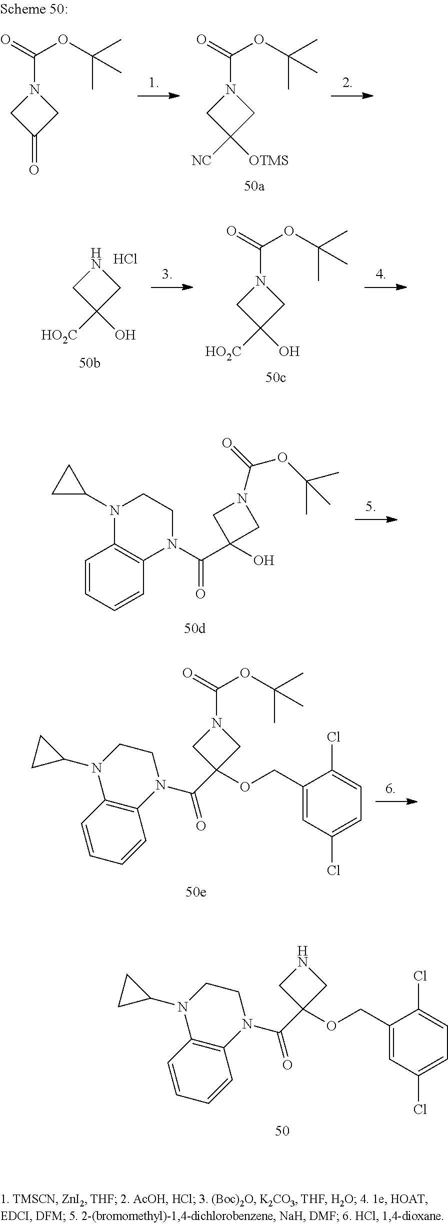

Intermediate 50a: tert-butyl 3-cyano-3-(trimethylsilyloxy)azetidine-1-carboxylate. tert-Butyl 3-oxoazetidine-1-carboxylate (10.0 g, 58.4 mmol), trimethylsilanecarbonitrile (8.68 g, 87.5 mmol, and diiodozinc (1.86 g, 5.83 mmol) were dissolved in tetrahydrofuran (100 mL). The resulting solution was stirred overnight at room temperature, then concentrated under reduced pressure. The residue was dissolved in 300 mL of ethyl acetate and washed twice with 5% sodium bicarbonate and once with H₂O. The organic layer was dried over anhydrous sodium sulfate and solvent removed under reduced pressure to give the title compound (11.8 g, 75%) as yellow oil, which was used without further purification.

Intermediate 50b: 3-hydroxyazetidine-3-carboxylic acid hydrochloride. 50a (11.8 g, 43.6 mmol) was added to acetic acid (20 mL), then concentrated hydrogen chloride (20 mL) was added dropwise with stirring at 0° C. The resulting solution was stirred for 4 h at 110° C., then the solvent removed under reduced pressure to give the title compound (6.6 g, 98%) as a yellow solid. MS (ES, m/z): 118 [M+H]⁺.

Intermediate 50c: 1-(tert-butoxycarbonyl)-3-hydroxyazetidine-3-carboxylic acid. 50b (6.06 g, 43.0 mmol) was dissolved in tetrahydrofuran/H₂O (60/60 mL), followed by the addition of potassium carbonate (18.0 g, 129 mmol) at 0° C. and di-tert-butyl dicarbonate (10.3 g, 47.2 mmol. The resulting mixture was stirred overnight at room temperature, then concentrated under reduced pressure. The pH value of the solution was adjusted to between 3 and 4 with aqueous HCl (3 M). The resulting solution was extracted four times with ethyl acetate and the organic layers combined and dried over anhydrous sodium sulfate and concentrated under reduced pressure. The crude residue was re-crystallized from petroleum ether/ethyl acetate (10/1) to give the title compound (4 g, 43%) as a white solid.

Intermediate 50d: tert-butyl 3-(4-cyclopropyl-1,2,3,4-tetrahydroquinoxaline-1-carbonyl)-3-hydroxyazetidine-1-carboxylate. 50c (900 mg, 4.14 mmol), le (150 mg, 0.86 mmol, 1.00 equiv), EDCI (248 mg, 1.29 mmol), and HOAT (176 mg, 1.29 mmol) were dissolved in N,N-dimethylformamide (3 mL) and the resulting solution was stirred for 4 h at room temperature. The solution was then diluted with of ethyl acetate (20 mL) and washed thrice with brine, dried over sodium sulfate, and the solvent removed under reduced pressure. The crude residue was purified by normal-phase flash column chromatography, using a gradient of ethyl acetate:petroleum ether (1:50 to 5:1) to give the title compound (150 mg, 47%) as a brown solid. MS (ES, m/z): 374 [M+H]⁺.

Intermediate 50e: tert-butyl 3-(4-cyclopropyl-1,2,3,4-tetrahydroquinoxaline-1-carbonyl)-3-(2,5-dichlorobenzyloxy)azetidine-1-carboxylate. 50d (160 mg, 0.43 mmol) was dissolved in N,N-dimethylformamide (5 mL). To the stirring solution was added 60% dispersion of sodium hydride in mineral oil (34.2 mg) in several batches at 0° C. The mixture was stirred for 20 min at 0° C., then 2-(bromomethyl)-1,4-dichlorobenzene (124 mg, 0.52 mmol) was added and the resulting mixture was stirred for 1 h at room temperature. The mixture was diluted with ethyl acetate (10 mL) and washed thrice with brine, then dried over anhydrous sodium sulfate and the solvent removed under reduced pressure. The crude residue was purified by preparative HPLC with a C18 silica gel stationary phase using a 10 min gradient (CH₃CN: H₂O 0.05% TFA 50:50 to 70:30) and detection by UV at 254 nm to provide the title compound TFA salt (18.1 mg, 8%) as a white solid. ¹H-NMR (400 MHz, CD₃OD, ppm): 7.61-7.59 (m, 1H), 7.35-7.15 (m, 2H), 7.09-6.90 (m, 2H), 6.85-6.73 (m, 2H), 4.58-4.17 (m, 4H), 4.10-4.06 (m, 1H), 3.88-3.81 (m, 2H), 3.77-3.73 (m, 1H), 3.50-3.42 (m, 2H), 2.50-2.35 (m, 1H), 1.46 (s, 9H), 0.81-0.72 (m, 2H), 0.57-0.50 (m, 1H), 0.25-0.11 (m, 1H). MS (ES, m/z): 532 [M+H]⁺.

Example 50: (4-cyclopropyl-3,4-dihydroquinoxalin-1(2H)-yl)(3-(2,5-dichlorobenzyloxyl)azetidin-3-yl)methanone. 50e (100 mg, 0.19 mmol) was dissolved in 1,4-1,4-dioxane (1.5 mL), then concentrated HCl (0.5 mL) was added at 0° C. The resulting solution was stirred for 1 h at room temperature, then the pH value of the solution was adjusted to 9 with sodium carbonate. The resulting solution was extracted thrice with ethyl acetate and the organic layers combined and washed with brine. The organic layer was dried over anhydrous sodium sulfate and solvent removed under reduced pressure. The crude residue was purified by preparative HPLC with a C18 silica gel stationary phase using a 10 min gradient (CH₃CN: H₂O 0.05% TFA 52:48 to 100:0) and detection by UV at 254 nm to provide the title compound bis TFA salt (9.6 mg, 12%) as a white solid. MS (ES, m/z): 432 [M+H]⁺. ¹H-NMR (400 MHz, CD₃OD) δ 7.62-7.56 (m, 1H), 7.40-6.93 (m, 5H), 6.73-6.69 (m, 1H), 4.58-4.36 (m, 2H), 4.22-4.11 (m, 2H), 3.87-3.65 (m, 4H), 3.50-3.41 (m, 2H), 2.40-2.30 (m, 1H), 0.81-0.72 (m, 2H), 0.60-0.40 (m, 1H), 0.30-0.20 (m, 1H).

### Example 51

### (4-cyclopropyl-3,4-dihydroquinoxalin-1(2H)-yl)(3-(2,5-dichlorobenzyloxy)-1-methylazetidin-3-yl)methanone

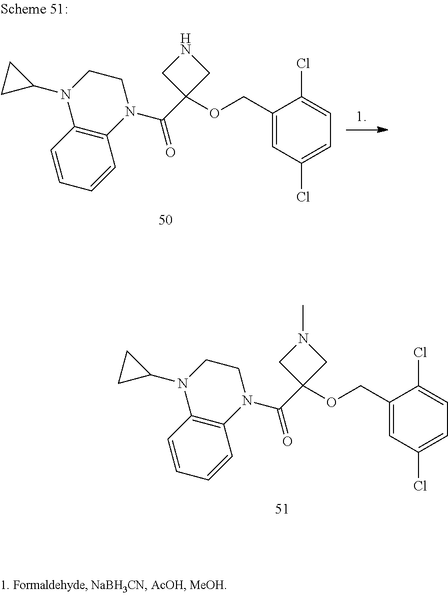 [SMILES: O=C(N1CCN(C2CC2)c2ccccc21)C1(OCc2cc(Cl)ccc2Cl)CNC1 ; CN1CC(OCc2cc(Cl)ccc2Cl)(C(=O)N2CCN(C3CC3)c3ccccc32)C1]

Example 51: (4-cyclopropyl-3,4-dihydroquinoxalin-1(2H)-yl)(3-(2,5-dichlorobenzyloxy)-1-methylazetidin-3-yl)methanone. 50 (50 mg, 0.12 mmol) and acetic acid (70 mg, 1.17 mmol) were dissolved in methanol (2 mL), followed by addition of 37% aqueous formaldehyde (30 mg, 0.37 mmol). The mixture was stirred for 1 h at room temperature, then NaBH₃CN (21 mg, 0.33 mmol was added and the solution was stirred for 1 h at room temperature. The solvent was removed under reduced pressure, then the residue dissolved in dichloromethane (10 mL) and washed with washed saturated aqueous NaHCO₃. The organic phase was dried over anhydrous sodium sulfate and the solvent removed under reduced pressure. The crude residue was purified by preparative HPLC with a C18 silica gel stationary phase using a 10 min gradient (CH₃CN: H₂O 0.05% TFA 44:56 to 100:0) and detection by UV at 254 nm to provide the title compound bis TFA salt (19.6 mg, 38%) as a brown solid. MS (ES, m/z): 446 [M+H]⁺. ¹H-NMR (300 MHz, CD₃OD) δ 7.73-7.58 (m, 0.6H), 7.48-7.26 (m, 2H), 7.25-7.14 (m, 1H), 7.12-6.96 (m, 1.7H), 6.92 (s, 0.7H), 6.75 (t, J=7.4 Hz, 1H), 4.65 (s, 1H), 4.44 (s, 2H), 3.89 (t, J=5.9 Hz, 1.4H), 3.69 (s, 0.6H), 3.45 (t, J=6.1 Hz, 1.4H), 3.11-2.86 (m, 3.6H), 2.48-2.23 (m, 1H), 0.90-0.78 (m, 0.6H), 0.79-0.65 (m, 1.4H), 0.63-0.49 (m, 0.6H), 0.24 (s, 1.4H).

### Example 52

### (4-cyclopropyl-3,4-dihydroquinoxalin-1(2H)-yl)(3-(2,5-dichlorobenzyloxy)-1-ethylazetidin-3-yl)methanone

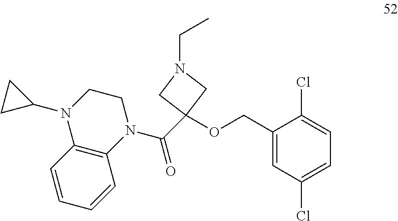 [SMILES: CCN1CC(OCc2cc(Cl)ccc2Cl)(C(=O)N2CCN(C3CC3)c3ccccc32)C1]

Example 52: (4-cyclopropyl-3,4-dihydroquinoxalin-1(2H)-yl)(3-(2,5-dichlorobenzyloxy)-1-ethylazetidin-3-yl)methanone. 52 can be synthesized in a similar manner as 51, substituting acetaldehyde for formaldehyde. Isolated as a bis-TFA salt. MS (ES, m/z): 460 [M+H]⁺; ¹H-NMR (300 MHz, CD₃OD) δ 7.71-7.59 (m, 0.6H), 7.48-7.14 (m, 3H), 7.14-6.93 (m, 1.7H), 6.92-6.65 (m, 1.7H), 4.74-4.09 (m, 5H), 3.89 (t, J=5.7 Hz, 1.3H), 3.71 (s, 0.7H), 3.51-3.24 (m, 2H), 2.46-2.23 (m, 1H), 1.37-1.13 (m, 3H), 0.91-0.77 (m, 0.7H), 0.70 (d, J=5.3 Hz, 1.3H), 0.55 (s, 0.7H), 0.19 (s, 1.3H).

### Example 53

### (4-cyclopropyl-3,4-dihydroquinoxalin-1(2H)-yl)(3-(2,5-dichlorobenzyloxy)-1-isopropylazetidin-3-yl)methanone

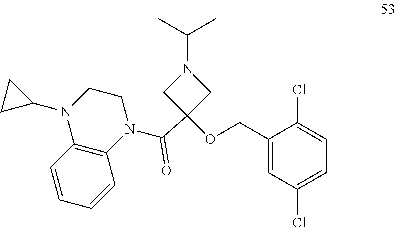 [SMILES: CC(C)N1CC(OCc2cc(Cl)ccc2Cl)(C(=O)N2CCN(C3CC3)c3ccccc32)C1]

Example 53: (4-cyclopropyl-3,4-dihydroquinoxalin-1(2H)-yl)(3-(2,5-dichlorobenzyloxy)-1-isopropylazetidin-3-yl)methanone. 53 can be synthesized in a similar manner as 51, substituting acetone for formaldehyde. Isolated as a bis TFA salt. MS (ES, m/z): 474 [M+H]⁺. ¹H-NMR (300 MHz, CD₃OD) δ 7.73-7.60 (m, 0.6H), 7.48-7.14 (m, 3H), 7.13-6.90 (m, 1.7H), 6.88-6.60 (m, 1.7H), 4.79-4.51 (m, 3H), 4.51-4.20 (m, 3H), 3.89 (s, 1.3H), 3.72 (s, 0.7H), 3.56-3.39 (m, 2H), 2.48-2.20 (m, 1H), 1.43-1.15 (m, 6H), 0.92-0.77 (m, 0.7H), 0.67 (d, J=5.5 Hz, 1.3H), 0.56 (s, 0.7H), 0.14 (s, 1.3H).

### Example 54

### 1-(3-(4-cyclopropyl-1,2,3,4-tetrahydroquinoxaline-1-carbonyl)-3-(2,5-dichlorobenzyloxyl)azetidin-1-yl)ethanone

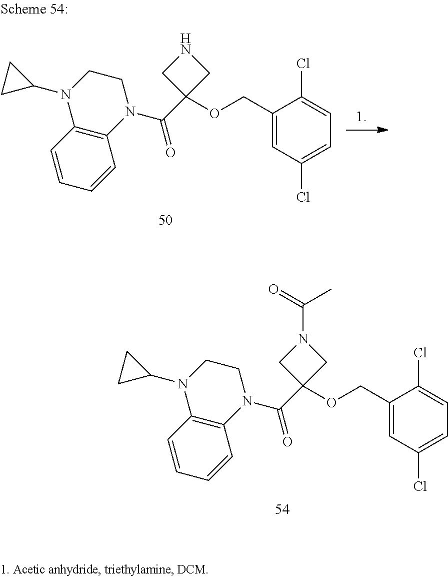 [SMILES: O=C(N1CCN(C2CC2)c2ccccc21)C1(OCc2cc(Cl)ccc2Cl)CNC1 ; CC(=O)N1CC(OCc2cc(Cl)ccc2Cl)(C(=O)N2CCN(C3CC3)c3ccccc32)C1]

Example 54: 1-(3-(4-cyclopropyl-1,2,3,4-tetrahydroquinoxaline-1-carbonyl)-3-(2,5-dichlorobenzyloxyl)azetidin-1-yl)ethanone. 50 (70 mg, 0.16 mmol), acetic anhydride (18 mg, 0.18 mmol), and triethylamine (49 mg, 0.48 mmol) were dissolved in DCM (2 mL) and stirred for 1 h at room temperature. The solution was then diluted with DCM and washed with brine, then dried over anhydrous sodium sulfate and the solvent removed under reduced pressure. The crude residue was purified by preparative HPLC with a C18 silica gel stationary phase using a 7 min gradient (CH₃CN: H₂O 0.03% NH₄OH 51:49 to 68:32) and detection by UV at 254 nm to provide the title compound (14.5 mg, 19%) as an off white solid. MS (ES, m/z): 474 [M+H]⁺. ¹H-NMR (300 MHz, CD₃OD) δ 7.45-7.32 (m, 1H), 7.31-7.12 (m, 2H), 7.06-6.82 (m, 2H), 6.60-6.52 (m, 2H), 4.57-4.39 (m, 2H), 4.39-4.16 (m, 2H), 4.16-4.03 (m, 1H), 3.73-3.53 (m, 3H), 3.34-3.21 (m, 2H), 2.18-2.11 (m, 1H), 1.73-1.68 (m, 3H), 0.73-0.60 (m, 1H), 0.48-0.33 (m, 1H), 0.20-0.10 (m, 1H).

### Example 55

### (4-cyclopropyl-3,4-dihydroquinoxalin-1(2H)-yl)(3-(2,5-dichlorobenzyloxy)-1-(methylsulfonyl)azetidin-3-yl)methanone

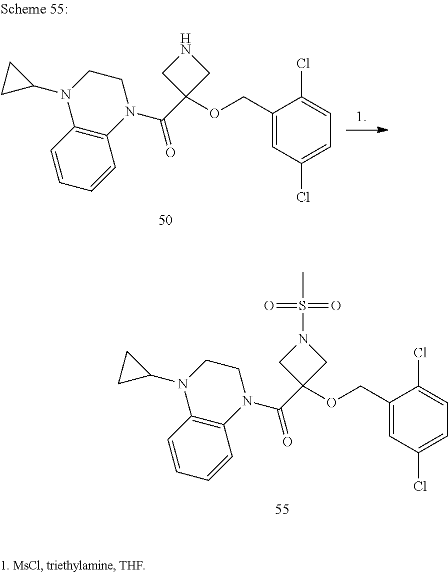 [SMILES: O=C(N1CCN(C2CC2)c2ccccc21)C1(OCc2cc(Cl)ccc2Cl)CNC1 ; CS(=O)(=O)N1CC(OCc2cc(Cl)ccc2Cl)(C(=O)N2CCN(C3CC3)c3ccccc32)C1]

Example 55: (4-cyclopropyl-3,4-dihydroquinoxalin-1(2H)-yl)(3-(2,5-dichlorobenzyloxy)-1-(methylsulfonyl)azetidin-3-yl)methanone. 50 (50 mg, 0.12 mmol) and triethylamine (35 mg, 0.35 mmol) were dissolved in tetrahydrofuran (3 mL), followed by the addition of methanesulfonyl chloride (16 mg, 0.14 mmol) dropwise with stirring at 0° C. The resulting solution was stirred for 10 min at 0° C. and for an additional 1 h at room temperature. The solution was then diluted with saturated aqueous sodium bicarbonate and extracted thrice with ethyl acetate and the organic layers combined and dried over anhydrous sodium sulfate and then the solvent removed under reduced pressure. The crude residue was purified by preparative HPLC with a C18 silica gel stationary phase using a 7 min gradient (CH₃CN: H₂O 0.03% NH₄OH 51:49 to 68:32) and detection by UV at 254 nm to provide the title compound TFA salt (8.9 mg, 15%) as a white solid. MS (ES, m/z): 510 [M+H]⁺. ¹H-NMR (400 MHz, CD₃OD) δ 7.75-7.62 (m, 1H), 7.37-7.04 (m, 4H), 6.90-6.74 (m, 2H), 4.62-4.26 (m, 5H), 3.97-3.74 (m, 3H), 3.50-3.34 (m, 2H), 3.20-2.96 (m, 3H), 2.40-2.34 (m, 1H), 0.83-0.72 (m, 1H), 0.72-0.57 (m, 1H), 0.57-0.50 (m, 1H), 0.20-0.10 (m, 1H).

### Example 56

### (4-cyclopropyl-3,4-dihydroquinoxalin-1(2H)-yl)(1-(2,5-dichlorobenzyl)amino)cyclobutyl)methanone

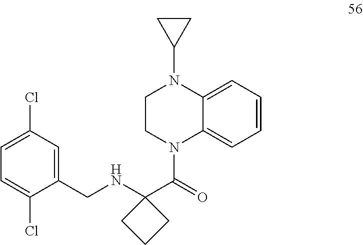 [SMILES: O=C(N1CCN(C2CC2)c2ccccc21)C1(NCc2cc(Cl)ccc2Cl)CCC1]

Example 56: (4-cyclopropyl-3,4-dihydroquinoxalin-1(2H)-yl)(1-(2,5-dichlorobenzyl)amino)cyclobutyl)methanone. Example 56 was prepared using the procedure described for the preparation of Example 26 except that 1-((t-butoxycarbonyl)amino)cyclobutanecarboxylic acid was used in place of Boc-1-aminocyclopropane-1-carboxylic acid. Isolated as the bis TFA salt. MS (ES, m/z): 430 [M+H]⁺. ¹H-NMR (400 MHz, CD₃OD) δ 7.60 (d, J=2.5 Hz, 1H), 7.48 (d, J=8.6 Hz, 1H), 7.44-7.36 (m, 2H), 7.24 (dd, J=8.3, 1.4 Hz, 1H), 7.19-7.11 (m, 1H), 6.73 (td, J=7.9, 1.4 Hz, 1H), 4.11 (s, 2H), 3.90 (t, J=5.5 Hz, 2H), 3.47 (t, J=5.8 Hz, 2H), 2.83-2.68 (m, 2H), 2.49-2.33 (m, 3H), 2.17-2.00 (m, 1H), 1.88-1.68 (m, 1H), 0.91-0.78 (m, 2H), 0.62-0.49 (m, 2H).

### Example 57

### (4-cyclopropyl-3,4-dihydroquinoxalin-1(2H)-yl)(1-(2,5-dichlorobenzyl)pyrrolidin-3-yl) methanone

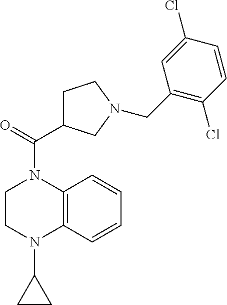 [SMILES: O=C(C1CCN(Cc2cc(Cl)ccc2Cl)C1)N1CCN(C2CC2)c2ccccc21]

Example 57: (4-cyclopropyl-3,4-dihydroquinoxalin-1(2H)-yl)(1-(2,5-dichlorobenzyl)pyrrolidin-3-yl) methanone. Example 57 was prepared using the procedure described for the preparation of Example 12 except that 1-(t-butoxycarbonyl) pyrrolidine-3-carboxylic acid was used in place of (S)-1-[(benzyloxy)carbonyl]pyrrolidine-2-carboxylic acid. Isolated as the bis TFA salt. MS (ES, m/z): 430 [M+H]⁺. ¹H-NMR (400 MHz, CD₃OD) δ 7.72 (s, 1H), 7.61-7.47 (m, 2H), 7.22 (dd, J=8.3, 1.5 Hz, 1H), 7.18 (t, J=7.6 Hz, 1H), 7.08 (d, J=7.6 Hz, 1H), 6.75 (t, J=7.2 Hz, 1H), 4.59 (s, 2H), 4.11-3.35 (m, 9H), 2.59-2.36 (m, 1H), 2.30-1.91 (m, 2H), 0.95-0.78 (m, 2H), 0.57 (s, 2H).

### Example 58

### (4-cyclopropyl-3,4-dihydroquinoxalin-1(2H)-yl)2-(3-methylbenzyl)pyrrolidin-1-yl)methanone

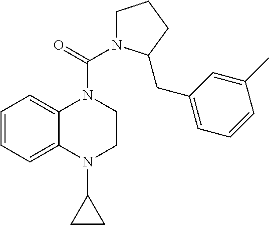 [SMILES: Cc1cccc(CC2CCCN2C(=O)N2CCN(C3CC3)c3ccccc32)c1]

Example 58: (4-cyclopropyl-3,4-dihydroquinoxalin-1(2H)-yl)2-(3-methylbenzyl)pyrrolidin-1-yl)methanone. Example 58 was prepared using the procedure described for the preparation of Example 28, except that 2-(3-methylbenzyl)pyrrolidine was used in place of 2-(2-chlorobenzyl)pyrrolidine. MS (ES, m/z): 376 [M+H]⁺; ¹H-NMR (400 MHz, CDCl₃) δ 7.21-7.10 (m, 2H), 7.05-6.88 (m, 5H), 6.71-6.60 (m, 1H), 4.25-4.07 (m, 2H), 3.53-3.34 (m, 2H), 3.33-3.20 (m, 2H), 3.15-3.06 (m, 2H), 2.52 (dd, J=12.4, 9.9 Hz, 1H), 2.45-2.36 (m, 1H), 2.30 (s, 3H), 1.93-1.45 (m, 4H), 0.87-0.76 (m, 2H), 0.73 (dd, J=9.5, 3.9 Hz, 1H), 0.51 (dd, J=10.0, 3.4 Hz, 1H).

### Example 59

### 3-(4-chloro-3-(((1-(4-cyclopropyl-1,2,3,4-tetrahydroquinoxaline-1-carbonyl)cyclopropyl)amino)methyl)phenyl)propanoic acid

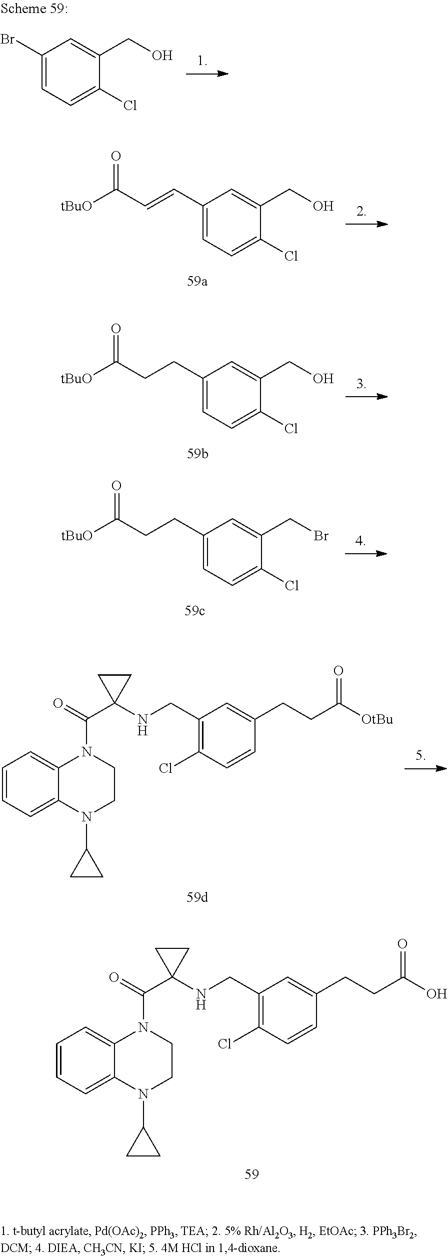 [SMILES: OCc1cc(Br)ccc1Cl ; CC(C)(C)OC(=O)/C=C/c1ccc(Cl)c(CO)c1 ; CC(C)(C)OC(=O)CCc1ccc(Cl)c(CO)c1 ; CC(C)(C)OC(=O)CCc1ccc(Cl)c(CBr)c1 ; CC(C)(C)OC(=O)CCc1ccc(Cl)c(CNC2(C(=O)N3CCN(C4CC4)c4ccccc43)CC2)c1 ; O=C(O)CCc1ccc(Cl)c(CNC2(C(=O)N3CCN(C4CC4)c4ccccc43)CC2)c1]

Intermediate 59a: t-Butyl 3-(4-chloro-3-(hydroxymethyl)phenyl)acrylate. To a mixture of (5-bromo-2-chlorophenyl)methanol (1.0 g, 4.51 mmol, 1 equiv.) and t-butyl acrylate (1.86 mL) in TEA (7.6 mL) were added palladium acetate (51 mg, 0.23 mmol, 0.05 equiv.) and PPh₃ (118 mg, 0.451 mmol, 0.1 equiv.). The mixture was stirred under N₂ at 90° C. overnight. The reaction mixture was concentrated under reduced pressure and purified by flash-column chromatography to give 1.03 g (85%) of t-butyl 3-(4-chloro-3-(hydroxymethyl)phenyl)acrylate as a clear syrup.

Intermediate 59b: t-butyl 3-(4-chloro-3-(hydroxymethyl)phenyl)propanoate. To a solution of t-butyl 3-(4-chloro-3-(hydroxymethyl)phenyl)acrylate (1.03 g, 3.84 mmol) in ethyl acetate (20 mL) was added Rh/Al₂O₃ (5%, 300 mg). The mixture was stirred at room temperature under H₂ for 3 h. More Rh/Al₂O₃ (5%, 150 mg) was added and the mixture was stirred at room temperature under H₂ overnight. The mixture was filtered and the filtrate was concentrated under reduced pressure to give 1g (96%) of t-butyl 3-(4-chloro-3-(hydroxymethyl)phenyl)propanoate as a clear syrup.

Intermediate 59c: t-butyl 3-(3-(bromomethyl)-4-chlorophenyl)propanoate. To a solution of t-butyl 3-(4-chloro-3-(hydroxymethyl)phenyl)propanoate (460 mg, 1.7 mmol, 1 equiv.) in DCM (12 mL) was added dibromo triphenylphosphorane (863 mg, 2.0 mmol, 1.2 equiv). The mixture was stirred at room temperature for 30 minutes, quenched with water, extracted with ether. The organic layer was washed with brine, dried over anhydrous sodium sulfate, concentrated under reduced pressure, and purified by flash-column chromatography to give 263 mg (46%) of t-butyl 3-(3-(bromomethyl)-4-chlorophenyl)propanoate as clear oil.

Intermediate 59d: t-butyl 3-(4-chloro-3-(((1-(4-cyclopropyl-1,2,3,4-tetrahydroquinoxaline-1-carbonyl)cyclopropyl)amino)methyl)phenyl)propanoate. To a mixture of (1-aminocyclopropyl)(4-cyclopropyl-3,4-dihydroquinoxalin-1-(2H)-yl)methanone HCl salt (26.4 mg, 0.08 mmol, 1 equiv), prepared form 26a by treating it with 4 M hydrochloric acid in 1,4-dioxane) and t-butyl 3-(3-(bromomethyl)-4-chlorophenyl)propanoate (32 mg, 0.096 mmol, 1.2 equiv) in acetonitrile (0.3 mL) were added DIEA (55.7 μL, 0.32 mmol, 4 equiv.) and KI (cat.). The mixture was stirred at 50° C. overnight, concentrated under reduced pressure, and purified by flash-column chromatography to give 31 mg (76%) of t-butyl 3-(4-chloro-3-(((1-(4-cyclopropyl-1,2,3,4-tetrahydroquinoxaline-1-carbonyl)cyclo-propyl)amino)methyl)phenyl)propanoate as yellow syrup. MS (ES, m/z): 510 [M+H]⁺.

Example 59: 3-(4-chloro-3-(((1-(4-cyclopropyl-1,2,3,4-tetrahydroquinoxaline-1-carbonyl)cyclopropyl)amino)methyl)phenyl)propanoic acid. To t-butyl 3-(4-chloro-3-(((1-(4-cyclopropyl-1,2,3,4-tetrahydroquinoxaline-1-carbonyl)cyclopropyl)amino)methyl)phenyl) propanoate (31 mg, 0.06 mmol) was added 4 M hydrochloric acid in 1,4-dioxane (1 mL). The mixture was stirred at room temperature for 3 h and concentrated to give 32 mg (crude) of 3-(4-chloro-3-(((1-(4-cyclopropyl-1,2,3,4-tetrahydroquinoxaline-1-carbonyl)cyclopropyl)-amino)methyl)phenyl)propanoic acid as a red solid. Some of the red solid (8 mg) was purified by preparative HPLC to give 5.9 mg of 3-(4-chloro-3-(((1-(4-cyclopropyl-1,2,3,4-tetrahydroquinoxaline-1-carbonyl)cyclopropyl)amino) methyl)phenyl)propanoic acid bis TFA salt as a yellow solid. MS (ES, m/z): 454 [M+H]⁺. ¹H-NMR (400 MHz, CDCl₃) δ 7.34-7.26 (m, 2H), 7.26-7.14 (m, 3H), 7.02 (d, J=2.0 Hz, 1H), 6.76 (td, J=7.6, 1.4 Hz, 1H), 4.07 (s, 2H), 3.89 (t, J=5.8 Hz, 2H), 3.43 (t, J=5.8 Hz, 2H), 2.86 (t, J=7.6 Hz, 2H), 2.57 (t, J=7.6 Hz, 2H), 2.48-2.36 (m, 1H), 1.39 (dd, J=7.8, 5.3 Hz, 2H), 1.22 (dd, J=7.8, 5.3 Hz, 2H), 0.89-0.77 (m, 2H), 0.57-0.44 (m, 2H).

### Example 60

### 3-(4-chloro-3-(((1-(4-cyclopropyl-1,2,3,4-tetrahydroquinoxaline-1-carbonyl)cyclopropyl)amino)methyl)phenyl)-N-methyl-N-((2S,3R,4R,5R)-2,3,4,5,6-pentahydroxyhexyl)propanamide

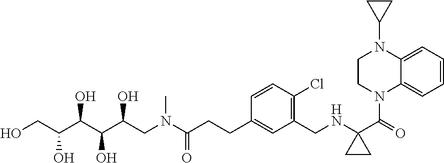 [SMILES: CN(C[C@H](O)[C@@H](O)[C@H](O)[C@H](O)CO)C(=O)CCc1ccc(Cl)c(CNC2(C(=O)N3CCN(C4CC4)c4ccccc43)CC2)c1]

Example 60: 3-(4-chloro-3-(((1-(4-cyclopropyl-1,2,3,4-tetrahydroquinoxaline-1-carbonyl)cyclopropyl)amino)methyl)phenyl)-N-methyl-N-((2S,3R,4R,5R)-2,3,4,5,6-penta-hydroxyhexyl)propanamide. To a mixture of Example 59 HCl salt (8.2 mg, 0.016 mmol, 1 equiv.) and N-methyl glucamine (3.9 mg, 0.02 mmol) in DMF (0.1 mL) were added HATU (7.6 mg, 0.02 mmol) and DIEA (17 uL, 0.1 mmol). The mixture was stirred at room temperature for 1 h and purified by preparative HPLC to give 7.4 mg (54%) of 3-(4-chloro-3-(((1-(4-cyclopropyl-1,2,3,4-tetrahydroquinoxaline-1-carbonyl)cyclopropyl)amino)methyl)-phenyl)-N-methyl-N-((2S,3R,4R,5R)-2,3,4,5,6-pentahydroxyhexyl)propanamide bis TFA salt as an off-white solid. MS (ES, m/z): 631 [M+H]⁺. ¹H-NMR (400 MHz, CD₃OD) δ 7.34-7.26 (m, 2H), 7.26-7.20 (m, 2H), 7.20-7.14 (m, 1H), 7.09 (dd, J=12.2, 1.9 Hz, 1H), 6.76 (ddd, J=9.2, 3.4, 1.7 Hz, 1H), 4.11 (d, J=8.8 Hz, 2H), 3.99-3.84 (m, 3H), 3.81-3.54 (m, 6H), 3.48-3.34 (m, 3H), 3.09 (s, 1.4H), 2.96 (s, 1.6H), 2.87 (t, J=7.5 Hz, 2H), 2.84-2.70 (m, 1H), 2.67 (t, J=7.5 Hz, 1H), 2.49-2.36 (m, 1H), 1.44-1.33 (m, 2H), 1.28-1.20 (m, 2H), 0.83 (dt, J=6.6, 1.6 Hz, 2H), 0.57-0.45 (m, 2H).

### Example 61

### (4-cyclopropyl-3,4-dihydroquinoxalin-1(2H)-yl)(1-(2,5-dichlorobenzyl)-1H-pyrrol-2-yl)methanone

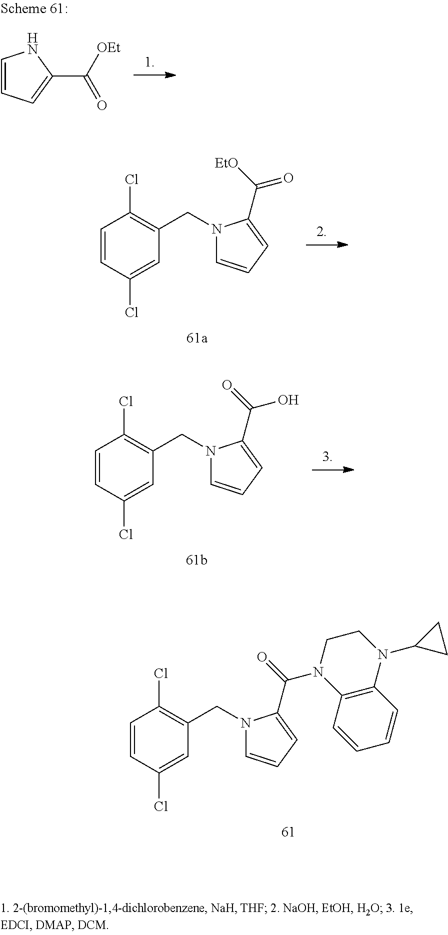 [SMILES: CCOC(=O)c1ccc[nH]1 ; CCOC(=O)c1cccn1Cc1cc(Cl)ccc1Cl ; O=C(O)c1cccn1Cc1cc(Cl)ccc1Cl ; O=C(c1cccn1Cc1cc(Cl)ccc1Cl)N1CCN(C2CC2)c2ccccc21]

Intermediate 61a: ethyl 1-(2,5-dichlorobenzyl)-1H-pyrrole-2-carboxylate. 2-(Bromomethyl)-1,4-dichlorobenzene (517 mg, 2.15 mmol) and ethyl 1H-pyrrole-2-carboxylate (300 mg, 2.16 mmol) were dissolved in tetrahydrofuran (20 mL). To the stirring solution was added sodium hydride dispersion in mineral oil (174 mg, 4.35 mmol) in several batches at 0-5° C. The resulting suspension was stirred overnight at room temperature then quenched by the addition of 5 mL of methanol. The solvent was removed under reduced pressure and the resulting residue purified by preparative TLC (ethyl acetate/petroleum ether 1:10) to give 61a (270 mg, 42%) as a white solid. MS (ES, m/z): 298 [M+H]⁺.

Intermediate 61b: 1-(2,5-dichlorobenzyl)-1H-pyrrole-2-carboxylic acid. 61a (200 mg, 0.67 mmol) and sodium hydroxide (539 mg, 13.47 mmol) were dissolved in ethanol/H₂O (8/4 mL) and stirred for 3 h at 85° C. The mixture was concentrated under reduced pressure and then diluted with 50 mL of dichloromethane. The pH value of the solution was adjusted to 3-4 with aqueous HCl (1 M). The organic layer was then washed twice with brine and dried over sodium sulfate, then solvent removed under reduced pressure to give 61b (150 mg, 83%) as a yellow solid, which was used directly without further purification.

Example 61: (4-cyclopropyl-3,4-dihydroquinoxalin-1(2H)-yl)(1-(2,5-dichlorobenzyl)-1H-pyrrol-2-yl)methanone. 61b (80 mg, 0.30 mmol, 1e (47 mg, 0.27 mmol), EDCI (77 mg, 0.40 mmol), and 4-dimethylaminopyridine (49 mg, 0.40 mmol) where dissolved in dichloromethane (3 mL). The resulting solution was stirred for 4 h at room temperature, then diluted with 20 mL of dichloromethane. The resulting mixture was washed twice with brine and dried over sodium sulfate, then concentrated under reduced pressure. The crude residue was purified by preparative HPLC with a C18 silica gel stationary phase using a 6 min gradient (CH₃CN: H₂O 0.05% TFA 32:68 to 50:50) and detection by UV at 254 nm to provide the title compound (23.7 mg, 19%) bis TFA salt as an off-white solid. MS (ES, m/z): 426 [M+H]⁺. ¹H-NMR (300 MHz, CD₃OD) δ 7.41 (d, J=8.7 Hz, 1H), 7.29 (dd, J=8.7, 2.4 Hz, 1H), 7.23 (d, J=8.4 Hz, 1H), 6.77-6.51 (m, 2H), 6.23 (d, J=3.9 Hz, 1H), 6.14 (t, J=3.3 Hz, 1H), 5.45 (s, 2H), 3.90 (t, J=5.7 Hz, 1H), 2.45-2.41 (m, 1H), 0.90-0.84 (m, 2H), 0.66-0.61 (m, 2H).

### Example 62

### (4-cyclopropyl-3,4-dihydroquinoxalin-1(2H)-yl)(1-(2,5-dichlorobenzyl)-1H-imidazol-2-yl)methanone

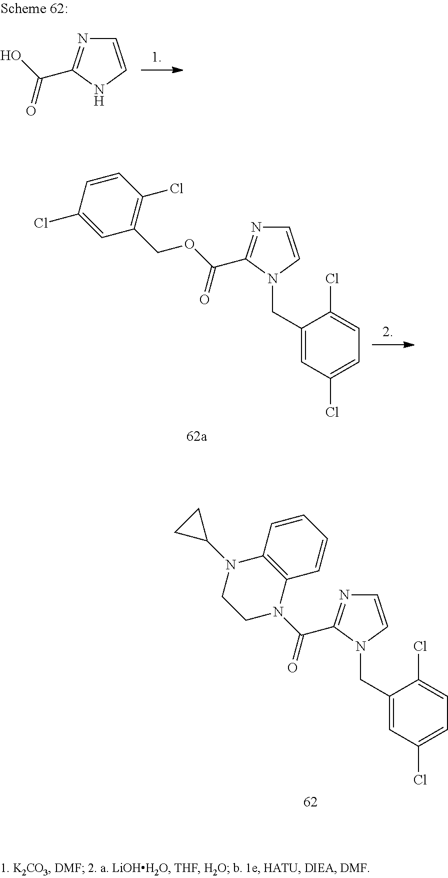 [SMILES: O=C(O)c1ncc[nH]1 ; O=C(OCc1cc(Cl)ccc1Cl)c1nccn1Cc1cc(Cl)ccc1Cl ; O=C(c1nccn1Cc1cc(Cl)ccc1Cl)N1CCN(C2CC2)c2ccccc21]

Intermediate 62a: 2,5-dichlorobenzyl 1-(2,5-dichlorobenzyl)-1H-imidazole-2-carboxylate. 1H-Imidazole-2-carboxylic acid (100 mg, 0.892 mmol), 1,4-dichloro-2-(chloromethyl)benzene (382 mg, 1.96 mmol), and K₂CO₃ (370 mg, 2.68 mmol) were combined in DMF. The suspension was stirred at 100° C. for 1 h, then added to 5% aqueous HCl and extracted with EtOAc. The organic phase was washed with saturated aqueous NaHCO₃, H₂O, and brine, then dried over Na₂SO₄ and the solvent removed under reduced pressure. The crude residue was purified by flash-column chromatography using a gradient of DCM:MeOH (100:0 to 98:2) to give 62a (290 mg, 76%) as a yellow oil.

Example 62: (4-cyclopropyl-3,4-dihydroquinoxalin-1(2H)-yl)(1-(2,5-dichlorobenzyl)-1H-imidazol-2-yl)methanone. 62a (290 mg, 0.674 mmol) and LiOH.H₂O (113 mg, 2.70 mmol) were dissolved in THF (3 mL) and H₂O (2 mL) and stirred at room temperature for 3 h. The solvent was removed under reduced pressure and the residue dissolved in EtOAc and MeOH and filtered, then solvent removed under reduced pressure. The crude residue was then dissolved in DMF, to which 1e (19 mg, 0.11 mmol), HATU (42 mg, 0.11 mmol), and DIEA (80 μL, 0.461 mmol) were added. The solution was stirred at room temperature for 1 h, then purified by preparative HPLC with a C18 silica gel stationary phase using a gradient of H₂O 0.05% TFA: CH₃CN 0.05% TFA (30:70 to 5:95) and detection by UV at 254 nm to give the title compound (23 mg, 5%) as the bis TFA salt. MS (ES, m/z): 427 [M+1]⁺. ¹H-NMR (400 MHz, CDCl₃) δ 7.40 (d, J=8.6 Hz, 1H), 7.37-7.29 (m, 2H), 7.24-7.17 (m, 2H), 7.11-7.02 (m, 2H), 6.78-6.33 (m, 2H), 5.41 (s, 2H), 4.02 (t, J=5.2 Hz, 2H), 3.50 (s, 2H), 2.48 (s, 1H), 0.87-0.81 (m, 2H), 0.68-0.62 (m, 2H).

### Example 63

### 1-cyclopropyl-4-([6-[(2,5-dichlorophenyl)methoxy]pyridin-2-yl]carbonyl)-1,2,3,4-tetrahydroquinoxaline

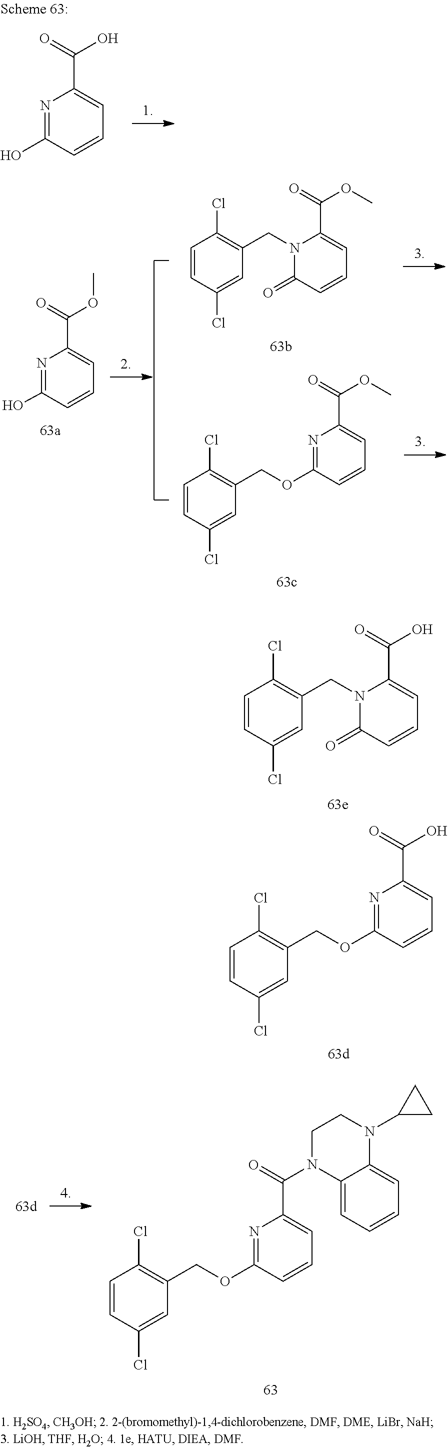

Intermediate 63a: methyl 6-hydroxypyridine-2-carboxylate. A solution of 6-hydroxypyridine-2-carboxylic acid (5.00 g, 35.94 mmol, 1.00 equiv) in methanol (100 mL) and sulfuric acid (20 mL). was stirred overnight at 65° C. The resulting reaction mixture was concentrated under reduced pressure, diluted with water (200 mL) and the solid precipitate was collected by filtration and washed with water and aqueous NaHCO₃. The filter cake was dissolved in ethyl acetate (20 mL) dried over anhydrous sodium sulfate and concentrated under reduced pressure to provide 2 g (36%) of the product as a white solid.

Intermediate 63b and 63c: methyl 1-(2,5-dichlorobenzyl)-6-oxo-1,6-dihydropyridine-2-carboxylate and methyl 6-(2,5-dichlorobenzyloxy)picolinate respectively. To a solution of methyl 6-hydroxypyridine-2-carboxylate (300 mg, 1.96 mmol, 1.00 equiv) in DMF (1 mL) and DME (3 mL) was added of sodium hydride (90 mg, 2.25 mmol, 1.15 equiv, 60%) at 0° C. followed by LiBr (339 mg, 3.90 mmol, 1.99 equiv) after a few minutes. The mixture was stirred for 15 min at room temperature then 2-(bromomethyl)-1,4-dichlorobenzene (900 mg, 3.75 mmol, 1.91 equiv) was added. The resulting solution was stirred overnight at 65° C. then quenched by the addition of 2 mL of H₂O. The resulting solution was extracted with ethyl acetate (2×10 mL) the organic layers combined and concentrated under reduced pressure. The resulting residue was applied onto a silica gel column and eluted with ethyl acetate/petroleum ether (1:2) to furnish 80 mg (13%) of the product 63b as a light yellow solid and 150 mg (25%) of the product 63c as a light yellow solid.

Intermediate 63d: 6-[(2,5-dichlorophenyl)methoxy]pyridine-2-carboxylic acid. A solution of methyl 6-[(2,5-dichlorophenyl)methoxy]pyridine-2-carboxylate 63c (150 mg, 0.48 mmol, 1.00 equiv), LiOH (10 mg, 0.42 mmol, 1.00 equiv) in tetrahydrofuran/H₂O (2:1 mL). The resulting solution was stirred for 2 h at room temperature then diluted with water (10 mL) and extracted with ethyl acetate (2×10 mL). The combined organic layers were dried over anhydrous sodium sulfate and concentrated under reduced pressure to provide 110 mg (77%) of the product as a light yellow solid.

Example 63: 1-cyclopropyl-4-([6-[(2,5-dichlorophenyl)methoxy]pyridin-2-yl]carbonyl)-1,2,3,4-tetrahydroquinoxaline. A solution of 6-[(2,5-dichlorophenyl)methoxy]-pyridine-2-carboxylic acid (110 mg, 0.37 mmol, 1.50 equiv), 1-cyclopropyl-1,2,3,4-tetrahydroquinoxaline (43 mg, 0.25 mmol, 1.00 equiv), HATU (187 mg, 0.49 mmol, 2.00 equiv), DIEA (64 mg, 0.50 mmol, 2.00 equiv) in N,N-dimethylformamide (2 mL) was stirred for 2 h at 40° C. The resulting solution was diluted with of ethyl acetate (20 mL), washed with brine (2×10 mL), dried over anhydrous sodium sulfate and concentrated under reduced pressure. The crude product (100 mg) was purified by preparative HPLC with the following conditions: Column, SunFire Preparative C18, 19*150 mm 5 μm; Mobile phase gradient, water containing 0.05% TFA: CH₃CN (48:52 to 25:75 over 6 min then up to 100% over 1 min); Detector, Waters 2545 UV detector at 254 and 220 nm. This resulted in 18 mg (16%) of the title compound ditrifluoroacetate salt as a yellow semi-solid. MS (ES, m/z): 454 [M+H]⁺. ¹H-NMR (300 MHz, CD₃OD) δ 7.80 (m, 1H), 7.37 (m, 2H), 7.30 (m, 2H), 7.20 (d, J=8.1 Hz, 1H), 6.95 (m, 2H), 6.40 (d, J=1.8 Hz, 2H), 3.94 (s, 1H), 3.49 (s, 2H), 2.45 (m, 1H), 1.19 (m, 3H), 0.87 (m, 2H), 0.69 (m, 2H).

### Example 64

### 1-cyclopropyl-4-([6-[(2,5-dichlorophenyl)methoxy]pyridin-2-yl]carbonyl)-1,2,3,4-tetrahydroquinoxaline

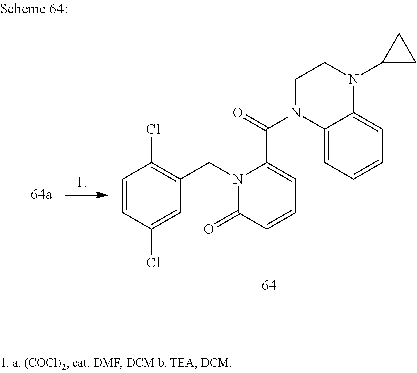 [SMILES: O=C(c1cccc(=O)n1Cc1cc(Cl)ccc1Cl)N1CCN(C2CC2)c2ccccc21]

Intermediate 64a: 1-[(2,5-dichlorophenyl)methyl]-6-oxo-1,6-dihydropyridine-2-carboxylic acid. A solution of methyl 1-[(2,5-dichlorophenyl)methyl]-6-oxo-1,6-dihydropyridine-2-carboxylate 63b (80 mg, 0.26 mmol, 1.00 equiv), LiOH (5 mg, 0.21 mmol, 0.81 equiv) in tetrahydrofuran: water (2:1 mL) was stirred for 2 h at room temperature then diluted with of water (5 mL). The resulting solution was extracted with ethyl acetate (2×10 mL) and the organic layers combined, dried over anhydrous sodium sulfate and concentrated under reduced pressure to give 50 mg (65%) of the product as a light yellow solid.

Example 64: 6-[(4-cyclopropyl-1,2,3,4-tetrahydroquinoxalin-1-yl)carbonyl]-1-[(2,5-dichlorophenyl)methyl]-1,2-dihydropyridin-2-one. To a solution of 1-[(2,5-dichlorophenyl)methyl]-6-oxo-1,6-dihydropyridine-2-carboxylic acid (50 mg, 0.17 mmol, 1.00 equiv) N,N-dimethylformamide (a catalytic amount), in dichloromethane (10 mL) was added oxalyl dichloride (1 mL). The resulting solution was stirred for 1 h at room temperature. The resulting mixture was concentrated under reduced pressure to provide 60 mg of the crude 1-[(2,5-dichlorophenyl)methyl]-6-oxo-1,6-dihydropyridine-2-carbonyl chloride as a yellow oil used without further purification. To a stirred at 0° C. solution of 1-cyclopropyl-1,2,3,4-tetrahydroquinoxaline (30 mg, 0.17 mmol, 1.00 equiv), triethylamine (0.5 mL) in dichloromethane (5 mL) was added 1-[(2,5-dichlorophenyl)methyl]-6-oxo-1,6-dihydropyridine-2-carbonyl chloride (60 mg, 1.00 equiv) in DCM. The resulting solution was stirred for 2 h at room temperature, concentrated under reduced pressure and the crude product (50 mg) was purified by preparative HPLC with the following conditions: Column, SunFire Preparative C18, 19*150 mm 5 μm; Mobile phase gradient, water containing 0.05% TFA: CH₃CN (65:35 to 48:52% over 10 min then to 100% in 1 min); Detector, Waters 2545 UV detector at 254 and 220 nm. This resulted in 13 mg (17%) of title compound trifluoroacetate salt as a yellow solid. MS (ES, m/z): 454 [M+H]⁺; ¹H-NMR (400 MHz, CD₃OD) δ 7.53 (s, 1H), 7.41 (6, J=6.6 Hz, 1H), 7.19 (m, 2H), 7.08 (m, 1H), 6.65 (m, 2H), 6.50 (m, 2H), 6.36 (s, 1H), 5.41 (m, 2H), 3.95 (s, 2H), 3.50 (s, 2H), 2.42 (s, 1H), 0.88 (d, J=3.6 Hz, 2H), 0.63 (s, 2H).

### Example 65

### 1-cyclopropyl-4-[[5-(2,5-dichlorophenoxy)-1,3-dimethyl-1H-pyrazol-4-yl]carbonyl]-1,2,3,4-tetrahydroquinoxaline

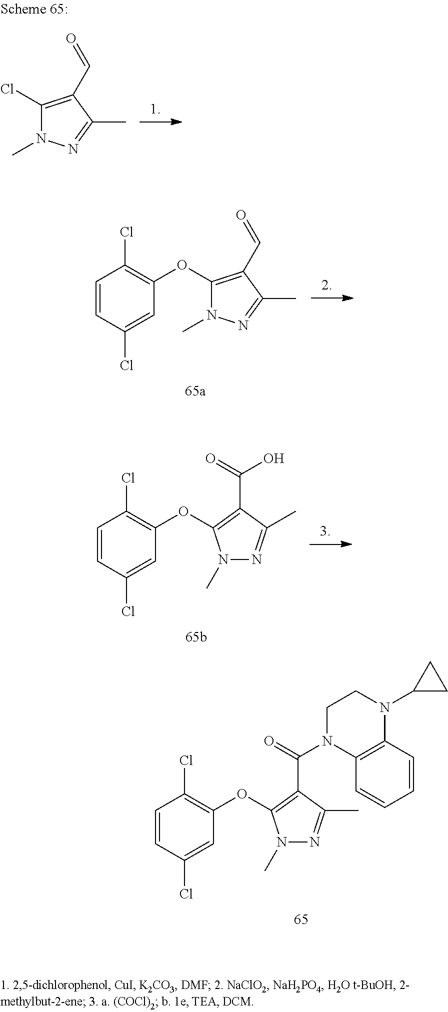

Intermediate 65a: 5-(2,5-dichlorophenoxy)-1,3-dimethyl-1H-pyrazole-4-carbaldehyde. A solution of 2,5-dichlorophenol (200 mg, 1.23 mmol, 1.95 equiv), 5-chloro-1,3-dimethyl-1H-pyrazole-4-carbaldehyde (100 mg, 0.63 mmol, 1.00 equiv), potassium carbonate (350 mg, 2.53 mmol, 4.02 equiv), Cu (25 mg, 0.39 mmol, 0.62 equiv), CuI (25 mg, 0.13 mmol, 0.21 equiv) in N,N-dimethylformamide (4 mL) was stirred overnight at 100° C. in an oil bath. The resulting reaction mixture was diluted with H₂O (20 mL) and extracted with ethyl acetate (3×20 mL) the combined organic layers were washed with water (20 mL) and brine (20 mL), dried over anhydrous sodium sulfate and concentrated under reduced pressure to furnish 120 mg (67%) of the product as brown oil.

Intermediate 65b: 5-(2,5-dichlorophenoxy)-1,3-dimethyl-1H-pyrazole-4-carboxylic acid. A solution of 5-(2,5-dichlorophenoxy)-1,3-dimethyl-1H-pyrazole-4-carbaldehyde (120 mg, 0.42 mmol, 1.00 equiv) NaH₂PO₄ (420 mg, 3.50 mmol, 8.32 equiv), NaClO₂ (360 mg, 4.00 mmol, 9.50 equiv) in tert-Butanol (6 mL), H₂O (6 mL) and 2-methylbut-2-ene (1 mL) was stirred overnight at room temperature. The resulting reaction mixture was diluted with H₂O (10 mL), extracted with ethyl acetate (3×20 mL) and the organic layers combined washed with brine (1×20 mL), dried over sodium sulfate and concentrated under reduced pressure to provide 110 mg (87%) of the product as a colorless oil.

Example 65: 1-cyclopropyl-4-[[5-(2,5-dichlorophenoxy)-1,3-dimethyl-1H-pyrazol-4-yl]carbonyl]-1,2,3,4-tetrahydroquinoxaline. To a stirred solution of 5-(2,5-dichlorophenoxy)-1,3-dimethyl-1H-pyrazole-4-carboxylic acid (100 mg, 0.33 mmol, 1.00 equiv) in dichloromethane (10 mL) was added oxalyl dichloride (10 mL) dropwise. The reaction mixture was stirred for 2 h at room temperature then concentrated under reduced pressure. The crude residue was dissolved in dichloromethane (5 mL) and added to a stirred 0° C. solution of 1-cyclopropyl-1,2,3,4-tetrahydroquinoxaline (80 mg, 0.46 mmol, 1.40 equiv), triethylamine (70 mg, 0.69 mmol, 2.00 equiv) in dichloromethane (10 mL). The resulting reaction mixture was allowed to warm to room temperature and stirred for 4 h then was diluted with H₂O (10 mL) and extracted with dichloromethane (3×10 mL) and the combined organic layers washed with brine (10 mL), dried over anhydrous sodium sulfate and concentrated under reduced pressure. The crude product (60 mg) was purified by Flash-Preparative-HPLC with the following conditions: Column, C18 silica gel; Mobile phase gradient CH₃CN in H₂O (containing 0.05% TFA) 5%-100% over 40 min; Detector, UV at 254 nm. This resulted in 16.1 mg (11%) of title compound trifluoroacetate salt as a light yellow solid. MS (ES, m/z): 457 [M+H]⁺. ¹H-NMR (300 MHz, CD₃OD) δ 7.44 (d, J=8.1 Hz, 1H), 7.10-7.18 (m, 2H), 6.96-7.04 (m, 1H), 6.79 (d, J=2.4 Hz, 1H), 6.66 (d, J=7.2 Hz, 1H), 6.51 (t, J=7.2 Hz, 1H), 3.78 (s, 2H), 3.56 (s, 3H), 3.19 (t, J=5.1 Hz, 2H), 2.31-2.38 (m, 1H), 2.15 (s, 3H), 0.73-0.83 (m, 2H), 0.53 (m, 2H).

### Example 66

### 1-cyclopropyl-4-([5-[(2,5-dichlorophenyl)methyl]-1,3-oxazol-4-yl]carbonyl)-1,2,3,4-tetrahydroquinoxaline

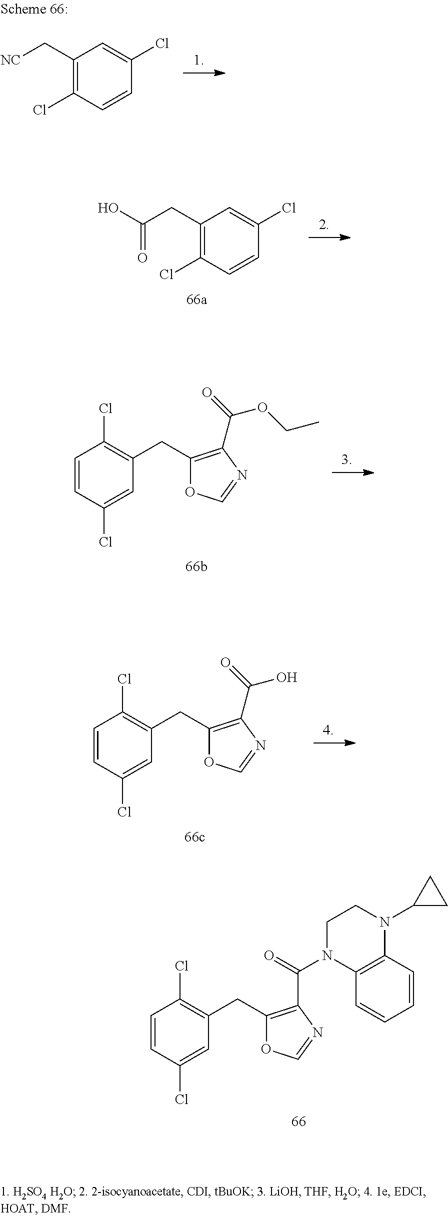 [SMILES: N#CCc1cc(Cl)ccc1Cl ; O=C(O)Cc1cc(Cl)ccc1Cl ; CCOC(=O)c1ncoc1Cc1cc(Cl)ccc1Cl ; O=C(O)c1ncoc1Cc1cc(Cl)ccc1Cl ; O=C(c1ncoc1Cc1cc(Cl)ccc1Cl)N1CCN(C2CC2)c2ccccc21]

Intermediate 66a: 2-(2,5-dichlorophenyl)acetic acid. To a stirred solution of 2-(2,5-dichlorophenyl)acetonitrile (700 mg, 3.76 mmol, 1.00 equiv) in water (6 mL) was added sulfuric acid (8 mL) dropwise. The resulting solution was stirred for 3 h at 110° C. in an oil bath, diluted with H₂O (100 mL), extracted with dichloromethane (3×50 mL) and the combined organic layers washed with brine (3×100 mL), dried over anhydrous sodium sulfate and concentrated under reduced pressure to provide 700 mg (91%) of the product as a white solid.

Intermediate 66b: ethyl 5-[(2,5-dichlorophenyl)methyl]-1,3-oxazole-4-carboxylate. A stirred solution of 2-(2,5-dichlorophenyl)acetic acid (1 g, 4.88 mmol, 1.00 equiv) and (2-ethoxy-2-oxoethyl)(methylidyne)azanium (560 mg, 4.91 mmol, 1.01 equiv), in N,N-dimethylformamide (10 mL) was treated with CDI (800 mg, 4.93 mmol, 1.01 equiv) followed by t-BuOK (55 mg, 0.49 mmol, 0.10 equiv). The resulting reaction mixture was stirred overnight at room temperature then diluted with H₂O (30 mL) and extracted with ethyl acetate (3×30 mL) and the organic layers combined. The combined organic layer was washed with brine (2×30 mL), dried over anhydrous sodium sulfate and concentrated under reduced pressure. The residue was applied onto a silica gel column and eluted with ethyl acetate/petroleum ether (1:5) to furnish 540 mg (37%) of the product as a yellow oil.

Intermediate 66c: 5-[(2,5-dichlorophenyl)methyl]-1,3-oxazole-4-carboxylic acid. A stirred solution of ethyl 5-[(2,5-dichlorophenyl)methyl]-1,3-oxazole-4-carboxylate (200 mg, 0.67 mmol, 1.00 equiv) and LiOH (50 mg, 2.09 mmol, 3.13 equiv) in tetrahydrofuran/H₂O (50/20 mL) was stirred overnight at 80° C. in an oil bath. The pH value of the resulting reaction mixture was adjusted to 3 with 1 M HCl extracted with ethyl acetate (3×20 mL) and the organic layers combined. The combined organic phase was washed with brine (20 mL), dried over anhydrous sodium sulfate and concentrated under reduced pressure to provide 130 mg (72%) of the product as a white solid.

Example 66: 1-cyclopropyl-4-([5-[(2,5-dichlorophenyl)methyl]-1,3-oxazol-4-yl]carbonyl)-1,2,3,4-tetrahydroquinoxaline. A solution of 5-[(2,5-dichlorophenyl)methyl]-1,3-oxazole-4-carboxylic acid (100 mg, 0.37 mmol, 1.00 equiv), 1-cyclopropyl-1,2,3,4-tetrahydroquinoxaline (60 mg, 0.34 mmol, 0.94 equiv), EDCI (75 mg, 0.39 mmol, 1.06 equiv) and HOAT (55 mg, 0.40 mmol, 1.10 equiv) in N,N-dimethylformamide (4 mL) was stirred overnight at room temperature. The reaction mixture was diluted with H₂O (20 mL), extracted with ethyl acetate (3×20 mL) and the organic layers combined. The combined organic phase was washed with brine (1×20 mL), dried over anhydrous sodium sulfate and concentrated under reduced pressure to provide crude product (150 mg) which was purified by preparative HPLC with the following conditions: Column, SunFire Preparative C18, 19*150 mm 5 μm; Mobile phase gradient, water containing 0.05% TFA: CH₃CN (48:52 to 32:68 over 10 min then to 100.0% in 1 min); Detector, Waters 2545 UV detector at 254 and 220 nm to provide 77.8 mg (49%) of title compound trifluoroacetate salt as a brown solid. MS (ES, m/z): 428 [M+H]⁺. ¹H-NMR (300 MHz, CDCl₃) δ 7.30 (s, 1H), 7.02 (m, 2H), 6.59 (t, J=6.9 Hz, 1H), 5.49 (s, 1H), 4.16 (s, 1H), 4.07 (t, J=5.7 Hz, 2H), 3.47 (t, J=5.7 Hz, 2H), 2.41-2.48 (m, 1H), 0.81-0.87 (m, 2H), 0.62-0.67 (m, 2H).

### Example 67

### (4-cyclopropyl-3,4-dihydroquinoxalin-1(2H)-yl)((2S)-1-(1-(2,5-dichlorophenyl)ethyl)pyrrolidin-2-yl)methanone

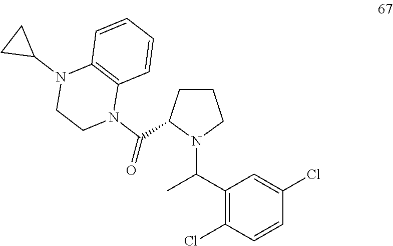 [SMILES: CC(c1cc(Cl)ccc1Cl)N1CCC[C@H]1C(=O)N1CCN(C2CC2)c2ccccc21]

Example 67: (4-cyclopropyl-3,4-dihydroquinoxalin-1(2H)-yl)((2S)-1-(1-(2,5-dichlorophenyl)ethyl)pyrrolidin-2-yl)methanone. Example 67 was prepared using the procedure described for the preparation of Example 12, except that 1-(2,5-dichlorophenyl)-ethyl methanesulfonate (prepared from 1-(2,5-dichlorophenyl)ethanol by standard mesylation methods) was used in place of 2-(bromomethyl)-1,4-dichlorobenzene. Two isomers were separated by preparative HPLC. Isomer 1: MS (ES, m/z): 444 [M+H]⁺. ¹H-NMR (400 MHz, CD₃OD) δ 7.67-7.59 (m, 1H), 7.49 (d, J=8.6 Hz, 0.2H), 7.42 (dd, J=8.6, 2.5 Hz, 0.8H), 7.39-7.34 (m, 0.3H), 7.32 (d, J=8.6 Hz, 0.7H), 7.28-7.15 (m, 1.7H), 7.08-6.98 (m, 0.3H), 6.77-6.67 (m, 1.5H), 6.63-6.53 (m, 0.5H), 5.21-5.07 (m, 0.3H), 5.05-4.92 (m, 0.7H), 4.58 (dd, J=10.6, 3.2 Hz, 1H), 4.08-3.90 (m, 2H), 3.74-3.38 (m, 2H), 3.26-3.18 (m, 1H), 2.90-2.78 (m, 1H), 2.63-1.98 (m, 5H), 1.80 (d, J=7.0 Hz, 0.6H), 1.64 (d, J=6.8 Hz, 2.4H), 0.97-0.80 (m, 2H), 0.69-0.49 (m, 2H). Isomer II δ 7.89-7.71 (m, 1H), 7.68-7.46 (m, 2.4H), 7.27 (d, J=4.2 Hz, 1.6H), 7.19-7.04 (m, 1H), 6.88-6.77 (m, 0.7H), 6.75-6.64 (m, 0.3H), 5.34-5.19 (m, 0.3H), 5.16-5.03 (m, 0.7H), 4.80-4.74 (m, 1H), 4.01-3.38 (m, 5H), 3.22-3.08 (m, 1H), 2.59-2.40 (m, 1H), 2.34-1.87 (m, 4H), 1.74 (d, J=6.8 Hz, 0.9H), 1.56 (d, J=6.9 Hz, 2.1H), 0.89 (dd, J=6.5, 2.0 Hz, 2H), 0.72-0.46 (m, 2H).

### Example 68

### (1-((5-(3-aminopropyl)-2-chlorobenzyl)amino)cyclopropyl)(4-cyclopropyl-3,4-dihydroquinoxalin-1(2H)-yl)methanone

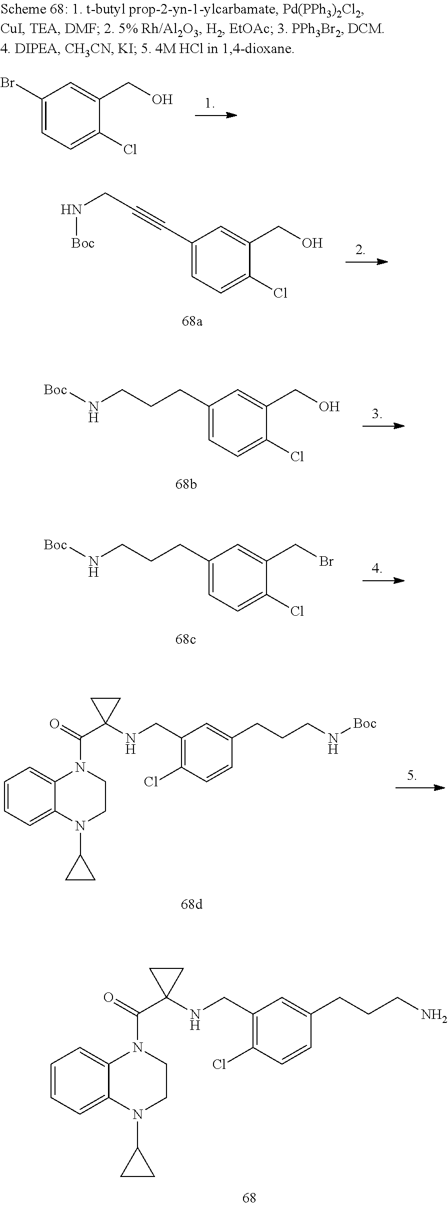 [SMILES: OCc1cc(Br)ccc1Cl ; CC(C)(C)OC(=O)NCC#Cc1ccc(Cl)c(CO)c1 ; CC(C)(C)OC(=O)NCCCc1ccc(Cl)c(CO)c1 ; CC(C)(C)OC(=O)NCCCc1ccc(Cl)c(CBr)c1 ; CC(C)(C)OC(=O)NCCCc1ccc(Cl)c(CNC2(C(=O)N3CCN(C4CC4)c4ccccc43)CC2)c1 ; NCCCc1ccc(Cl)c(CNC2(C(=O)N3CCN(C4CC4)c4ccccc43)CC2)c1]

Intermediate 68a: t-Butyl(3-(4-chloro-3-(hydroxymethyl)phenyl)prop-2-yn-1-yl)cabamate. To a mixture of (5-bromo-2-chlorophenyl)methanol (1.00 g, 4.51 mmol), t-butyl prop-2-yn-1-ylcabamate (0.84 g, 5.4 mmol) and TEA (5.2 mL) in DMF (3.2 mL) were added Pd(PPh₃)₂Cl₂ (158 mg, 0.226 mmol) and CuI (86 mg, 0.45 mmol). The mixture was stirred under N₂ at 50° C. overnight. The reaction mixture was diluted with EtOAc, washed with water (2×) and brine (1×), dried over anhydrous sodium sulfate, concentrated under reduced pressure and purified by flash-column chromatography to give 477 mg (36%) of 68a as a pale yellow syrup.

Intermediate 68b: t-butyl(3-(4-chloro-3-(hydroxymethyl)phenyl)propyl)-carbamate. To a solution of t-butyl(3-(4-chloro-3-(hydroxymethyl)phenyl)prop-2-yn-1-yl)cabamate (477 mg, 1.61 mmol) in ethyl acetate (15 mL) was added Rh/Al₂O₃ (5%, 160 mg). The mixture was stirred at room temperature under H₂ for 6 h. More Rh/A1 (5%, 160 mg) was added and the mixture was stirred at room temperature under an atmosphere of H₂ overnight. The mixture was filtered and the filtrate was concentrated under reduced pressure to give 463 mg (96%) of 68b as a brown syrup.

Intermediate 68c: t-butyl(3-(3-(bromomethyl)-4-chlorophenyl)propyl)-carbamate. To a solution of t-butyl(3-(4-chloro-3-(hydroxymethyl)phenyl)propyl)carbamate (190 mg, 0.63 mmol) in DCM (4.5 mL) was added dibromo triphenylphosphorane (295 mg, 0.7 mmol). The mixture was stirred at room temperature for 30 minutes, quenched with water, and extracted with ether. The organic layer was washed with brine (1×), dried with anhydrous sodium sulfate, concentrated under reduced pressure, and purified by flash-column chromatography to give 45 mg (20%) of 68c as a yellow syrup.

Intermediate 68d: t-butyl(3-(4-chloro-3-(((1-(4-cyclopropyl-1,2,3,4-tetra-hydroquinoxaline-1-carbonyl)cyclopropyl)amino)methyl)phenyl)propyl)carbamate. To a mixture of (1-aminocyclopropyl)(4-cyclopropyl-3,4-dihydroquinoxalin-1-(2H)-yl)methanone HCl salt (20 mg, 0.060 mmol, prepared from 26a by treating it with 4 M hydrochloric acid in 1,4-dioxane) and t-butyl(3-(3-(bromomethyl)-4-chlorophenyl)propyl)carbamate (22 mg, 0.06 mmol) in acetonitrile (0.25 mL) were added DIPEA (43 uL, 0.25 mmol) and KI (cat.). The mixture was stirred at 50° C. overnight, concentrated under reduced pressure, and purified by flash-column chromatography to give 21 mg (64%) of 68d as a yellow syrup. MS (ES, m/z): 539 [M+H]⁺.

Example 68: (1-((5-(3-aminopropyl)-2-chlorobenzyl)amino)cyclopropyl)(4-cyclopropyl-3,4-dihydroquinoxalin-1(2H)-yl)methanone. To t-butyl(3-(4-chloro-3-(((1-(4-cyclopropyl-1,2,3,4-tetrahydroquinoxaline-1-carbonyl)cyclopropyl)amino)methyl)phenyl)-propyl)carbamate (21 mg, 0.039 mmol) was added a 4 M hydrochloric acid solution in 1,4-dioxane (1 mL). The mixture was stirred at room temperature for 30 min and concentrated under reduced pressure to give 21 mg (crude) of (1-((5-(3-aminopropyl)-2-chlorobenzyl)-amino)cyclopropyl)(4-cyclopropyl-3,4-dihydroquinoxalin-1(2H)-yl)methanon as a red solid. Some of the red solid (4 mg) was purified by preparative HPLC to give 3 mg of the title compound TFA salt as a yellow solid. MS (ES, m/z): 439 [M+H]⁺. ¹H-NMR (400 MHz, CDCl₃) δ 7.30 (dd, J=7.9, 1.4 Hz, 1H), 7.26 (d, J=8.1 Hz, 1H), 7.20 (dd, J=8.3, 1.4 Hz, 1H), 7.16-7.07 (m, 2H), 6.87 (d, J=1.8 Hz, 1H), 6.73 (td, J=7.7, 1.4 Hz, 1H), 3.88 (t, J=5.6 Hz, 2H), 3.76 (s, 2H), 3.40 (t, J=5.8 Hz, 2H), 2.90 (t, J=8.0 Hz, 2H), 2.62 (t, J=8.0 Hz, 2H), 2.47-2.35 (m, 1H), 1.99-1.81 (m, 2H), 1.40 (q, J=4.6 Hz, 2H), 1.01 (q, J=4.6 Hz, 2H), 0.84-0.72 (m, 2H), 0.53-0.37 (m, 2H).

### Example 69

### 3-(3-(4-chloro-3-(((1-(4-cyclopropyl-1,2,3,4-tetrahydroquinoxaline-1-carbonyl)cyclopropyl)amino)methyl)phenyl)propyl)-1-methyl-1-((2S,3R,4R,5R)-2,3,4,5,6-pentahydroxyhexyl)urea

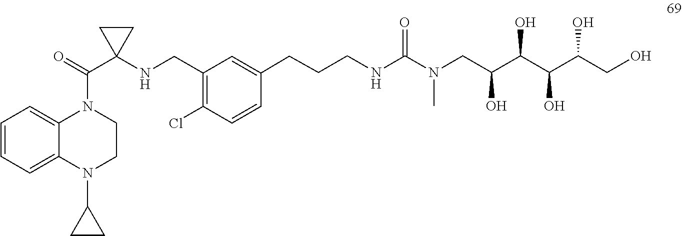 [SMILES: CN(C[C@H](O)[C@@H](O)[C@H](O)[C@H](O)CO)C(=O)NCCCc1ccc(Cl)c(CNC2(C(=O)N3CCN(C4CC4)c4ccccc43)CC2)c1]

Example 69: 3-(3-(4-chloro-3-(((1-(4-cyclopropyl-1,2,3,4-tetrahydro-quinoxaline-1-carbonyl)cyclopropyl)amino)methyl)phenyl)propyl)-1-methyl-1-((2S,3R,4R,5R)-2,3,4,5,6-pentahydroxyhexyl)urea. To a mixture of Example 68 (16.2 mg, 0.037 mmol) in THF (0.2 mL) was added N,N′-disuccinimidyl carbonate (10.4 mg, 0.041 mmol). The mixture was stirred at room temperature for 1 h. To the mixture was added N-methyl-D-glucamine (10.8 mg, 0.055 mmol). The reaction mixture was stirred at 60° C. for 4 h and more N,N′-disuccinimidyl carbonate (10.4 mg, 0.041 mmol) was added. The mixture was stirred at 60° C. overnight, concentrated under reduced pressure, and purified by preparative HPLC to give 8.8 mg (27%) of the title compound TFA salt as a yellow syrup. MS (ES, m/z): 660 [M+H]⁺. ¹H-NMR (400 MHz, CD₃OD) δ 7.39 (d, J=8.1 Hz, 1H), 7.30-7.23 (m, 4H), 7.22-7.15 (m, 1H), 6.76 (td, J=7.5, 1.4 Hz, 1H), 4.37 (s, 2H), 3.99-3.87 (m, 3H), 3.81-3.59 (m, 5H), 3.50-3.41 (m, 3H), 3.40-3.33 (m, 1H), 3.14 (t, J=6.9 Hz, 2H), 2.95 (s, 3H), 2.65 (t, J=7.6 Hz, 2H), 2.49-2.38 (m, 1H), 1.86-1.73 (m, 2H), 1.44-1.32 (m, 4H), 0.92-0.77 (m, 2H), 0.63-0.50 (m, 2H).

### Example 70

### 3-(4-chloro-3-(((1-(4-cyclopropyl-1,2,3,4-tetrahydroquinoxaline-1-carbonyl)cyclopropyl)amino)methyl)phenyl)-N-((2S,3R,4R,5R)-2,3,4,5,6-pentahydroxyhexyl)propanamide

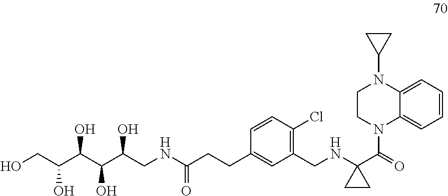 [SMILES: O=C(CCc1ccc(Cl)c(CNC2(C(=O)N3CCN(C4CC4)c4ccccc43)CC2)c1)NC[C@H](O)[C@@H](O)[C@H](O)[C@H](O)CO]

Example 70: 3-(4-chloro-3-(((1-(4-cyclopropyl-1,2,3,4-tetrahydroquinoxaline-1-carbonyl)cyclopropyl)amino)methyl)phenyl)-N-((2S,3R,4R,5R)-2,3,4,5,6-pentahydroxy-hexyl)propanamide. Example 70 was prepared using the procedure described for the preparation of Example 60, except that D-glutamine was used in place of N-methyl-D-glucamine MS (ES, m/z): 617 [M+H]⁺.

### Example 71

### 3-(4-chloro-3-(((1-(4-cyclopropyl-1,2,3,4-tetrahydroquinoxaline-1-carbonyl)cyclopropyl)amino)methyl)phenyl)-N-methyl-N-((2S,3R,4S,5R)-2,3,4,5,6-pentahydroxyhexyl)propanamide

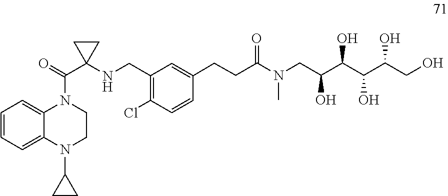 [SMILES: CN(C[C@H](O)[C@@H](O)[C@@H](O)[C@H](O)CO)C(=O)CCc1ccc(Cl)c(CNC2(C(=O)N3CCN(C4CC4)c4ccccc43)CC2)c1]

Example 71: 3-(4-chloro-3-(((1-(4-cyclopropyl-1,2,3,4-tetrahydroquinoxaline-1-carbonyl)cyclopropyl)amino)methyl)phenyl)-N-methyl-N-((2S,3R,4S,5R)-2,3,4,5,6-penta-hydroxyhexyl)propanamide. Example 71 was prepared using the procedure described for the preparation of Example 60, except that 1-Deoxy-1-(methylamino)-D-galactitol was used in place of N-methyl-D-glucamine MS (ES, m/z): 631 [M+1-1]⁺.

### Example 72

### N-(2-cyclobutoxyphenyl)-1-(2,5-dichlorobenzyloxy)-N-methylcyclopropanecarboxamide

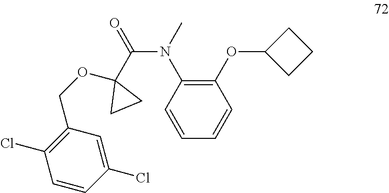 [SMILES: CN(C(=O)C1(OCc2cc(Cl)ccc2Cl)CC1)c1ccccc1OC1CCC1]

Example 72: N-(2-cyclobutoxyphenyl)-1-(2,5-dichlorobenzyloxy)-N-methylcyclopropanecarboxamide. Example 72 was synthesized in an analogous fashion to Example 36 using 2-cyclobutoxy-N-methylaniline (which was made in an analogous fashion to 39e, substituting cyclobutanol for cyclopropylmethanol) in place of 2-methoxy-N-methylaniline. MS (ES, m/z): 420 [M+1-1]⁺.

### Example 73

### 1-cyclopropyl-4-[[5-(2,5-dichlorophenoxy)-1,3-dimethyl-1H-pyrazol-4-yl]carbonyl]-1,2,3,4-tetrahydroquinoxaline

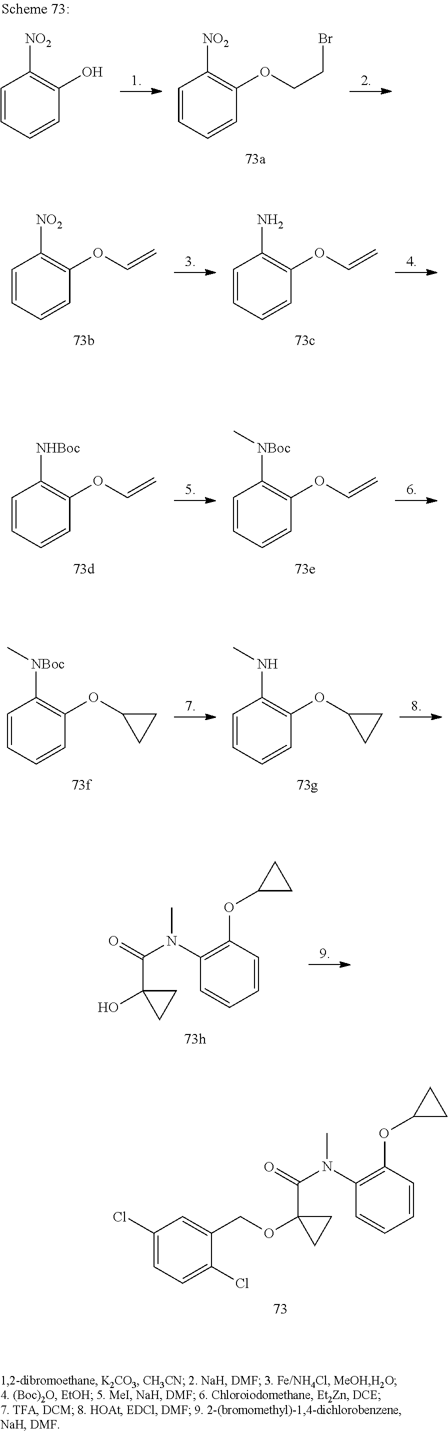

Intermediate 73a 1-(2-bromoethoxy)-2-nitrobenzene: A solution of 2-nitrophenol (1.00 g, 7.19 mmol, 1.00 equiv) 1,2-dibromoethane (4.00 g, 21.3 mmol, 3.00 equiv), potassium carbonate (1.90 g, 13.8 mmol, 2.00 equiv) in CH₃CN (30 mL) was stirred for 3 h at 90° C. The resulting reaction mixture was concentrated under reduced pressure, dissolved in of ethyl acetate (200 mL) and washed with brine (3×50 mL). The combined organic phase was dried over anhydrous sodium sulfate and concentrated under reduced pressure. This resulted in 1 g (57%) of 73a as a green oil.

Intermediate 73b 1-(ethenyloxy)-2-nitrobenzene: To a stirred 0-5° C. solution of 1-(2-bromoethoxy)-2-nitrobenzene (550 mg, 2.24 mmol, 1.00 equiv) in DMF (6 mL) was added sodium hydride (180 mg, 4.50 mmol, 2.00 equiv) in portions. The resulting reaction mixture was stirred overnight at room temperature, diluted with of ethyl acetate (50 mL) and washed with brine (3×20 mL), dried over anhydrous sodium sulfate, and concentrated under reduced pressure to provide 200 mg (54%) of 73b as a yellow oil.

Intermediate 73c 2-(ethenyloxy)aniline: To a 60° C. solution of 1-(ethenyloxy)-2-nitrobenzene (300 mg, 1.82 mmol, 1.00 equiv) in methanol (10 mL) was added a solution of ammonium chloride (970 mg, 18.1 mmol, 10.0 equiv) in water (3 mL) followed by the addition of iron powder (1 g, 17.91 mmol, 10.00 equiv) in portions. The resulting reaction mixture was stirred for 2 h, solids were removed by filtration and the filtrate concentrated under reduced pressure. The residue was dissolved in 20 mL of ethyl acetate, washed with brine (2×20 mL), the organic phase dried over anhydrous sodium sulfate, and concentrated under reduced pressure to provide 200 mg (81%) of 73c as a brown oil.

Intermediate 73d tert-butyl N-[2-(ethenyloxy)phenyl]carbamate: A solution of 2-(ethenyloxy)aniline (230 mg, 1.70 mmol, 1.00 equiv) in ethanol (2 mL) and di-tert-butyl dicarbonate (446 mg, 2.04 mmol, 1.20 equiv) was stirred overnight at room temperature. The resulting reaction mixture was concentrated under reduced pressure and the residue was purified by silica gel chromatography eluting with ethyl acetate/petroleum ether (1:200) to provide 200 mg (50%) of 73d as a yellow oil.

Intermediate 73e tert-butyl N-[2-(ethenyloxy)phenyl]-N-methylcarbamate: To a stirred 0° C. solution of tert-butyl N-[2-(ethenyloxy)phenyl]carbamate (190 mg, 0.81 mmol, 1.00 equiv) in DMF (2 mL) was added sodium hydride (49 mg, 1.2 mmol, 1.5 equiv) in several batches. The reaction mixture was stirred for 0.5 h at 0° C. and iodomethane (230 mg, 1.62 mmol, 2.00 equiv) was added dropwise with stirring. The resulting reaction mixture was allowed to warm to room temperature and stirred for 0.5 h then diluted with 50 mL of ethyl acetate. The resulting organic solution was washed with brine (3×20 mL) dried over anhydrous sodium sulfate, and concentrated under reduced pressure to provide 150 mg (75%) of 73e as a brown oil.

Intermediate 73f tert-butyl N-(2-cyclopropoxyphenyl)-N-methylcarbamate: To a stirred 0° C. solution of tert-butyl N-[2-(ethenyloxy)phenyl]-N-methylcarbamate (150 mg, 0.60 mmol, 1.00 equiv) in 1,2-dichloroethane (10 mL) was added chloro(iodo)methane (382 mg, 2.17 mmol, 3.60 equiv) followed by dropwise addition of diethylzinc (1.5 mL, 2.40 equiv, 1.0 M). The resulting reaction mixture was allowed to warm to 25° C., stirred overnight, then quenched by the addition of 20 mL of aqueous NH₄Cl. The resulting reaction mixture was extracted with dichloromethane (2×20 mL) and the combined organic layers were dried over anhydrous sodium sulfate and concentrated under reduced pressure to provide 150 mg of 73f as a brown oil used without further purification.

Intermediate 73g 2-cyclopropoxy-N-methylaniline: A stirred solution of tert-butyl N-(2-cyclopropoxyphenyl)-N-methylcarbamate (30 mg, 0.11 mmol, 1.0 equiv) in 1,4-dioxane (1.5 mL) and concentrated hydrogen chloride (0.5 mL) was stirred for 1 h at 25° C. The pH value of the reaction mixture was adjusted to 9 with sodium carbonate then extracted with ethyl acetate (2×50). The combined organic layers were dried over anhydrous sodium sulfate and concentrated under reduced pressure to provide 10 mg of 73g as a brown oil, which was used without further purification.

Intermediate 73h N-(2-cyclopropoxyphenyl)-1-hydroxy-N-methylcyclopropane-1-carboxamide: A stirred solution of 1-hydroxycyclopropane-1-carboxylic acid (100 mg, 0.98 mmol, 1.00 equiv), 2-cyclopropoxy-N-methylaniline (176 mg, 1.08 mmol, 1.10 equiv), EDCI (283 mg, 1.48 mmol, 1.50 equiv) and HOAt (200 mg, 1.47 mmol, 1.50 equiv) in DMF (2 mL) was stirred overnight at room temperature. The resulting reaction mixture was diluted with ethyl acetate (50 mL) washed with brine (4×20 mL), dried over anhydrous sodium sulfate, and concentrated under reduced pressure to provide 80 mg (33%) of 73h as a white solid. MS (ES, m/z): 248 [M+H]⁺.

Example 73 1-cyclopropyl-4-[[5-(2,5-dichlorophenoxy)-1,3-dimethyl-1H-pyrazol-4-yl]carbonyl]-1,2,3,4-tetrahydroquinoxaline: Example 73 was prepared as described for example 8, substituting 73h for 8c. MS (ES, m/z): 406 [M+H]⁺; ¹H-NMR (300 MHz, CD₃OD) δ 7.26-7.10 (m, 5H), 6.89-6.84 (m, 1H), 6.40 (m, 2H), 4.43-4.39 (m, 1H), 4.32-4.27 (m, 1H), 3.10 (s, 3H), 1.40-1.36 (m, 1H), 1.30-1.10 (m, 1H), 1.10-0.90 (m, 1H), 0.89-0.88 (m, 1H), 0.67-0.50 (m, 3H), 0.35-0.32 (m, 1H).

### Example 74

### 1[[(2S)-1-[[3-chloro-5-(trifluoromethoxy)phenyl]methyl]pyrrolidin-2-yl]carbonyl]-4-cyclopropyl-1,2,3,4-tetrahydroquinoxaline

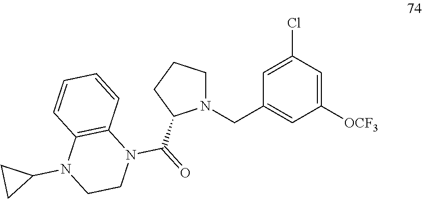 [SMILES: O=C([C@@H]1CCCN1Cc1cc(Cl)cc(OC(F)(F)F)c1)N1CCN(C2CC2)c2ccccc21]

Example 74 1-[[(2S)-1-[[3-chloro-5-(trifluoromethoxy)phenyl]methyl]-pyrrolidin-2-yl]carbonyl]-4-cyclopropyl-1,2,3,4-tetrahydroquinoxaline: Example 74 was prepared as described for example 12, substituting 1-(bromomethyl)-3-chloro-5-(trifluoromethoxy)benzene for 2-(bromomethyl)-1,4-dichlorobenzene. This resulted in 33.4 mg (31%) of the title compound as colorless oil. MS (ES, m/z): 480 [M+H]⁺; ¹H-NMR (400 MHz, CD₃OD) δ 7.20 (s, 1H), 7.15-7.18 (m, 4H), 6.90 (s, 1H), 6.70-6.72 (m, 1H), 4.88 (s, 1H), 4.60 (m, 1H), 3.88 (m, 1H), 3.66-3.69 (m, 1H), 3.32-3.50 (m, 3H), 3.03-3.16 (m, 1H), 2.37-2.45 (m, 2H), 1.81-1.95 (m, 4H), 0.82-0.84 (m, 2H), 0.51 (s, 2H).

### Example 75

### 3-[2,5-dichloro-4-([1-[(4-cyclopropyl-1,2,3,4-tetrahydroquinoxalin-1-yl)carbonyl]cyclopropyl]methoxy)phenyl]-N-[(2S,3R,4R,5R)-2,3,4,5,6-pentahydroxyhexyl]propanamide

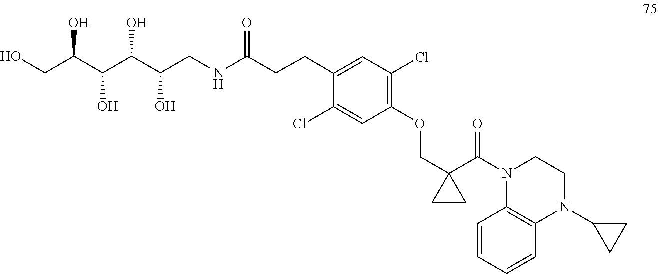 [SMILES: O=C(CCc1cc(Cl)c(OCC2(C(=O)N3CCN(C4CC4)c4ccccc43)CC2)cc1Cl)NC[C@H](O)[C@@H](O)[C@H](O)[C@H](O)CO]

Example 75 3-[2,5-dichloro-4-([1-[(4-cyclopropyl-1,2,3,4-tetrahydro-quinoxalin-1-yl)carbonyl]cyclopropyl]methoxy)phenyl]-N-[(2S,3R,4R,5R)-2,3,4,5,6-pentahydroxyhexyl]propanamide: Example 75 was prepared as described for example 34 substituting (2R,3R,4R,5S)-6-aminohexane-1,2,3,4,5-pentol for (2R,3R,4R,5 S)-6-(methylamino)hexane-1,2,3,4,5-pentaol. This resulted in 83.3 mg (31%) of the title compound trifluoroacetate salt as an off-white solid. MS (ES, m/z): 652 [M+H]⁺; ¹H-NMR (300 MHz, CD₃OD) δ 7.38-7.42 (d, J=7.8 Hz, 1H), 7.33 (s, 1H), 7.08-7.11 (m, 2H), 6.70-6.73 (m, 1H), 6.61 (s, 1H), 3.61-3.90 (m, 11H), 3.37-3.44 (m, 3H), 2.92-2.96 (m, 2H), 2.46-2.48 (m, 2H), 2.23-2.25 (m, 1H), 1.35-1.37 (m, 2H), 0.95-0.99 (m, 2H), 0.64-0.68 (m, 2H), 0.17-0.18 (m, 2H).

### Example 76

### 1-cyclopropyl-4-[[(2S,4R)-1-[(2,5-dichlorophenyl)methyl]-4-methoxypyrrolidin-2-yl]carbonyl]-1,2,3,4-tetrahydroquinoxaline

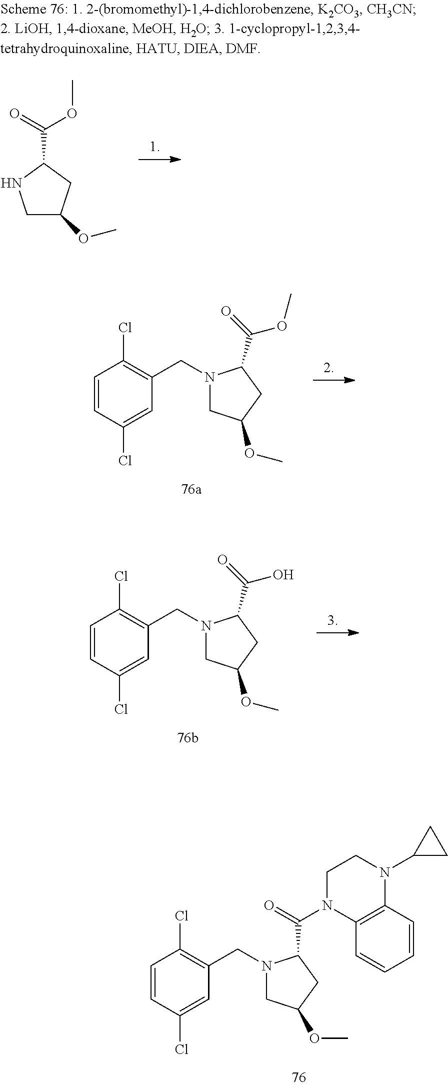 [SMILES: COC(=O)[C@@H]1C[C@@H](OC)CN1 ; COC(=O)[C@@H]1C[C@@H](OC)CN1Cc1cc(Cl)ccc1Cl ; CO[C@@H]1C[C@@H](C(=O)O)N(Cc2cc(Cl)ccc2Cl)C1 ; CO[C@@H]1C[C@@H](C(=O)N2CCN(C3CC3)c3ccccc32)N(Cc2cc(Cl)ccc2Cl)C1]

Intermediate 76a methyl(2S,4R)-1-[(2,5-dichlorophenyl)methyl]-4-methoxypyrrolidine-2-carboxylate: A solution of methyl(2S,4R)-4-methoxypyrrolidine-2-carboxylate (150 mg, 0.94 mmol, 1.00 equiv), 2-(bromomethyl)-1,4-dichlorobenzene (243 mg, 1.01 mmol, 1.07 equiv), and potassium carbonate (390 mg, 2.82 mmol, 2.99 equiv) in CH₃CN (5 mL) was stirred overnight at room temperature. The resulting solution was diluted with 30 ml of ethyl acetate then washed with 2×20 mL of brine, dried over sodium sulfate, and concentrated under reduced pressure. The residue was applied onto a silica gel column and eluted with a mobile phase of petroleum ether/ethyl acetate (20:1) to provide 260 mg (87%) of 76a as a colorless oil. MS (ES, m/z): 318 [M+H]⁺. ¹H-NMR (300 MHz, CDCl₃) δ 7.52-7.49 (m, 1H), 7.27-7.19 (m, 1H), 7.15-7.11 (m, 1H), 4.02-3.91 (m, 2H), 3.82-3.77 (m, 1H), 3.67 (s, 3H), 3.60 (t, J=7.8 Hz, 1H), 3.38-3.32 (m, 1H), 3.26 (s, 3H), 2.54-2.49 (m, 1H), 2.19-2.15 (m, 2H).

Intermediate 76b (2S,4R)-1-[(2,5-dichlorophenyl)methyl]-4-methoxypyrrolidine-2-carboxylic acid: A solution of methyl (2S,4R)-1-[(2,5-dichlorophenyl)methyl]-4-methoxypyrrolidine-2-carboxylate (260 mg, 0.82 mmol, 1.00 equiv) in 1,4-dioxane/MeOH/H₂O (6 mL) was added lithium hydroxide (69 mg, 1.6 mmol, 2.0 equiv). The resulting solution was stirred for 1 h at 80° C. in an oil bath. The pH value of the solution was adjusted to 6 with hydrogen chloride (2 M). The resulting mixture was concentrated under reduced pressure to furnish 300 mg (crude) of 76b as a colorless oil, which was used without further purification. MS (ES, m/z): 304 [M+H]⁺.

Example 76 1-cyclopropyl-4-[[(2S,4R)-1-[(2,5-dichlorophenyl)methyl]-4-methoxypyrrolidin-2-yl]carbonyl]-1,2,3,4-tetrahydroquinoxaline: A solution of (2S,4R)-1-[(2,5-dichlorophenyl)methyl]-4-methoxypyrrolidine-2-carboxylic acid (104 mg, 0.34 mmol, 1.00 equiv), 1-cyclopropyl-1,2,3,4-tetrahydroquinoxaline (60 mg, 0.34 mmol, 1.0 equiv), HATU (262 mg, 0.69 mmol, 2.00 equiv), and DIEA (89 mg, 0.69 mmol, 2.0 equiv) in DMF (3 mL) was stirred overnight at room temperature. The resulting reaction mixture was diluted with ethyl acetate (30 mL). The resulting mixture was washed with brine (3×20 mL), dried over anhydrous sodium sulfate, and concentrated under reduced pressure. The crude product (100 mg) was purified by preparative HPLC with the following conditions: Column, SunFire preparative C18, 19*150 mm 5 nm; Column, SunFire preparative C18, 19*150 mm 5 nm; mobile phase gradient, water containing 0.05% TFA: CH₃CN (40% CH₃CN up to 56% in 6 min); detector, Waters 2545 UV detector at 254 and 220 nm to provide 44.2 mg (28%) of the title compound bis-trifluoroacetate salt as a white solid. MS (ES, m/z): 460 [M+H]⁺; ¹H-NMR (300 MHz, CD₃OD) δ 7.69 (s, 1H), 7.47 (s, 2H), 7.23 (d, J=3 Hz, 2H), 6.98 (d, J=9 Hz, 1H), 6.76 (m, 1H), 4.83 (m, 1H), 4.68 (d, J=12 Hz, 1H), 4.55 (d, J=15 Hz, 1H), 4.09 (s, 1H), 3.96 (t, J=6 Hz, 1H), 3.79 (d, J=6 Hz, 1H), 3.58-3.49 (m, 2H), 3.43-3.33 (m, 2H), 3.27 (s, 3H), 3.14 (t, J=6 Hz, 1H), 2.44 (t, J=3 Hz, 2H), 2.12-2.07 (m, 1H), 1.86 (m, 1H), 0.88-0.84 (m, 2H), 0.66-0.63 (m, 1H), 0.50-0.46 (m, 1H).

### Example 77

### 1-cyclopropyl-4-[[(2S,4S)-1-[(2,5-dichlorophenyl)methyl]-4-methoxypyrrolidin-2-yl]carbonyl]-1,2,3,4-tetrahydroquinoxaline

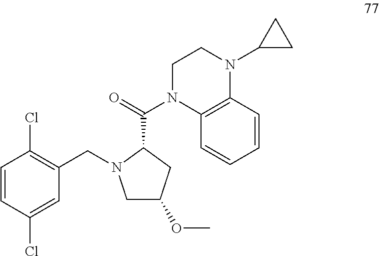 [SMILES: CO[C@H]1C[C@@H](C(=O)N2CCN(C3CC3)c3ccccc32)N(Cc2cc(Cl)ccc2Cl)C1]

Example 77 1-cyclopropyl-4-[[(2S,4S)-1-[(2,5-dichlorophenyl)methyl]-4-methoxypyrrolidin-2-yl]carbonyl]-1,2,3,4-tetrahydroquinoxaline: Example 77 was prepared as described for example 76, substituting (2S,4S)-1-[(2,5-dichlorophenyl)methyl]-4-methoxypyrrolidine-2-carboxylate for methyl (2S,4R)-1-[(2,5-dichlorophenyl)methyl]-4-methoxypyrrolidine-2-carboxylate. This resulted in 55.7 mg (35%) of the title compound bis-trifluoroacetate salt as a white solid. MS (ES, m/z): 460 [M+H]⁺; ¹H-NMR (400 MHz, CD₃OD) δ 7.79 (s, 1H), 7.54 (s, 2H), 7.28 (d, J=8 Hz, 2H), 7.06 (d, J=8 Hz, 1H), 6.80 (m, 1H), 4.95 (m, 1H), 4.67-4.56 (m, 2H), 4.07 (d, J=12 Hz, 2H), 3.87 (d, J=12 Hz, 1H), 3.47-3.41 (m, 3H), 3.28 (s, 3H), 3.24-3.15 (m, 1H), 2.52 (m, 2H), 1.92 (m, 1H), 0.94-0.89 (m, 2H), 0.70 (m, 1H), 0.51 (m, 1H).

### Example 78

### 1-cyclopropyl-4-[[(2S,4R)-1-[(2,5-dichlorophenyl)methyl]-4-fluoropyrrolidin-2-yl]carbonyl]-1,2,3,4-tetrahydroquinoxaline

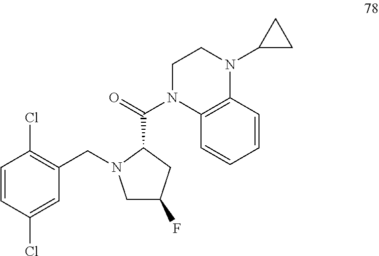 [SMILES: O=C([C@@H]1C[C@@H](F)CN1Cc1cc(Cl)ccc1Cl)N1CCN(C2CC2)c2ccccc21]

Example 78 1-cyclopropyl-4-[[(2S,4R)-1-[(2,5-dichlorophenyl)methyl]-4-fluoropyrrolidin-2-yl]carbonyl]-1,2,3,4-tetrahydroquinoxaline: Example 78 was prepared as described for example 76, substituting methyl (2S,4R)-4-fluoropyrrolidine-2-carboxylate for methyl (2S,4R)-1-[(2,5-dichlorophenyl)methyl]-4-methoxypyrrolidine-2-carboxylate. This resulted in 19.3 mg (13%) of the title compound bis-trifluoroacetate salt as a blue solid. MS (ES, m/z): 448 [M+H]⁺; ¹H-NMR (400 MHz, CD₃OD) δ 7.71 (s, 1H), 7.49 (s, 2H), 7.27 (d, J=4 Hz, 2H), 7.06 (d, J=8 Hz, 1H), 6.80 (m, 1H), 5.44-5.30 (m, 1H), 5.07-5.03 (m, 1H), 4.68-4.52 (m, 1H), 3.95-3.63 (m, 4H), 3.46-3.40 (m, 2H), 3.27-3.23 (m, 1H), 2.48 (s, 1H), 2.28-2.23 (m, 2H), 0.92-0.86 (m, 2H), 0.66-0.54 (m, 2H).

### Example 79

### 1-cyclopropyl-4-([4-[(2,5-dichlorophenyl)methoxy]oxan-4-yl]carbonyl)-1,2,3,4-tetrahydroquinoxaline

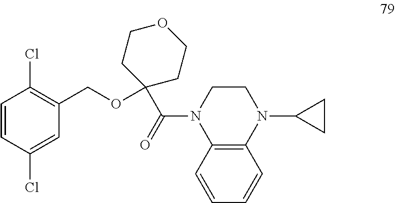 [SMILES: O=C(N1CCN(C2CC2)c2ccccc21)C1(OCc2cc(Cl)ccc2Cl)CCOCC1]

Example 79 1-cyclopropyl-4-([4-[(2,5-dichlorophenyl)methoxy]oxan-4-yl]carbonyl)-1,2,3,4-tetrahydroquinoxaline: Example 79 was prepared as described for example 8, substituting oxan-4-one for cyclopentanone. This resulted in 4.8 mg (4%) of the title compound trifluoroacetate salt as a white solid. MS (ES, m/z): 461 [M+H]⁺; ¹H-NMR (400 MHz, CD₃OD) δ 7.30-7.40 (m, 4H), 7.02-7.13 (m, 2H), 6.66-6.70 (m, 1H), 4.55 (s, 2H), 4.07 (s, 2H), 3.78-3.87 (m, 4H), 2.12-2.38 (m, 5H), 0.76-0.80 (m, 2H), 0.50 (s, 2H).

### Example 80

### 3-[2,5-dichloro-4-([1-[(4-cyclopropyl-1,2,3,4-tetrahydroquinoxalin-1-yl)carbonyl]cyclopropoxyl]methyl)phenyl]-N-methyl-N-[(2S,3R,4R,5R)-2,3,4,5,6-pentahydroxyhexyl]propanamide

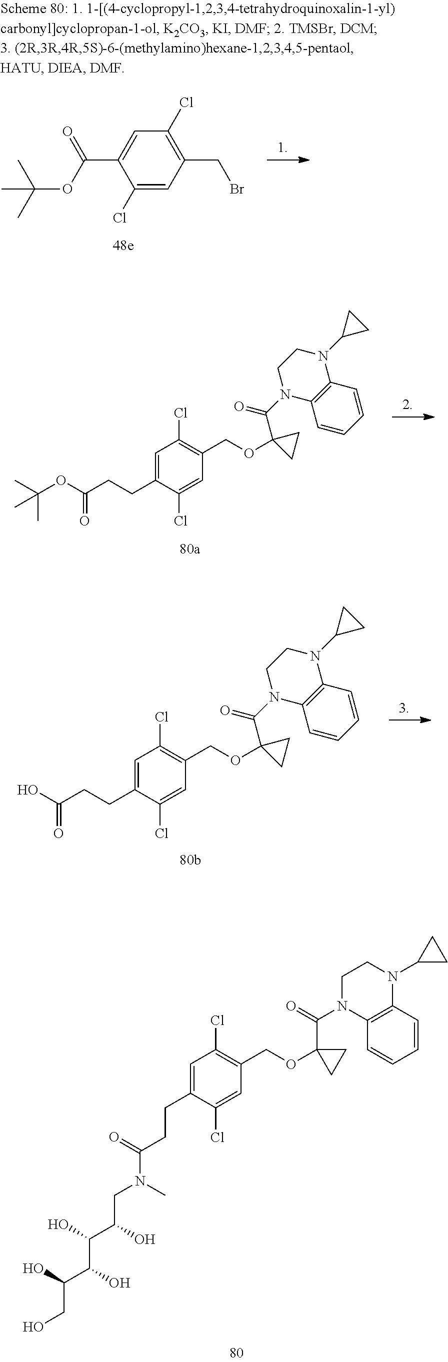 [SMILES: CC(C)(C)OC(=O)c1cc(Cl)c(CBr)cc1Cl ; CC(C)(C)OC(=O)CCc1cc(Cl)c(COC2(C(=O)N3CCN(C4CC4)c4ccccc43)CC2)cc1Cl ; O=C(O)CCc1cc(Cl)c(COC2(C(=O)N3CCN(C4CC4)c4ccccc43)CC2)cc1Cl ; CN(C[C@H](O)[C@@H](O)[C@H](O)[C@H](O)CO)C(=O)CCc1cc(Cl)c(COC2(C(=O)N3CCN(C4CC4)c4ccccc43)CC2)cc1Cl]

Intermediate 80a 3-[2,5-dichloro-4-([1-[(4-cyclopropyl-1,2,3,4-tetrahydro-quinoxalin-1-yl)carbonyl]cyclopropoxy]methyl)phenyl]propanoate: A stirred solution of tert-butyl 3-[4-(bromomethyl)-2,5-dichlorophenyl]propanoate (200 mg, 0.54 mmol, 1.00 equiv), 1-[(4-cyclopropyl-1,2,3,4-tetrahydroquinoxalin-1-yl)carbonyl]cyclopropan-1-ol, 9a (140 mg, 0.54 mmol, 1.00 equiv), potassium carbonate (150 mg, 1.09 mmol, 2.00 equiv), and KI (18 mg, 0.11 mmol, 0.20 equiv) dissolved in DMF (2 mL) in a sealed tube was stirred overnight at 30° C. in an oil bath. The resulting reaction mixture was concentrated under reduced pressure and purified by preparative TLC, with a mobile phase of petroleum ether/ethyl acetate (5:1) to provide 130 mg (44%) of 80a as a light-yellow oil.

Intermediate 80b 3-[2,5-dichloro-4-([1-[(4-cyclopropyl-1,2,3,4-tetrahydro-quinoxalin-1-yl)carbonyl]cyclopropoxy]methyl)phenyl]propanoic acid: To a stirred solution of tert-butyl 3-[2,5-dichloro-4-([1-[(4-cyclopropyl-1,2,3,4-tetrahydroquinoxalin-1-yl)carbonyl]cyclopropoxy]methyl)phenyl]propanoate (130 mg, 0.24 mmol, 1.00 equiv) in dichloromethane (2 mL) was added TMSBr (2 mL). The resulting reaction mixture was stirred for 2 h at room temperature, then concentrated under reduced pressure, then diluted with H₂O (50 mL). The resulting mixture was extracted with ethyl acetate (3×30 mL) and the organic layers combined, dried over sodium sulfate, and concentrated under reduced pressure to provide 100 mg (86%) of 80b as a light yellow solid.

Example 80 3-[2,5-dichloro-4-([1-[(4-cyclopropyl-1,2,3,4-tetrahydro-quinoxalin-1-yl)carbonyl]cyclopropoxy]methyl)phenyl]-N-methyl-N-[(2S,3R,4R,5R)-2,3,4,5,6-pentahydroxyhexyl]propanamide: A solution of 3-[2,5-dichloro-4-([1-[(4-cyclopropyl-1,2,3,4-tetrahydroquinoxalin-1-yl)carbonyl]cyclopropoxy]methyl)phenyl]-propanoic acid (100 mg, 0.20 mmol, 1.00 equiv), (2R,3R,4R,5S)-6-(methylamino)hexane-1,2,3,4,5-pentol (60 mg, 0.31 mmol, 1.50 equiv), HATU (117 mg, 0.31 mmol, 1.50 equiv), and DIEA (53 mg, 0.41 mmol, 2.00 equiv) in DMF (2 mL) was stirred overnight at room temperature. The resulting reaction mixture was concentrated under reduced pressure and the crude product residue (150 mg) was purified by preparative HPLC with the following conditions: Column, SunFire preparative C18, 19*150 mm 5 μm; mobile phase gradient, water containing 0.05% TFA: CH₃CN (38.0% CH₃CN to 56.0% over 6 min); Detector, Waters 2545 UV detector at 254 and 220 nm to provide 95 mg (70%) of the title compound trifluoroacetate salt as an off-white solid. MS (ES, m/z): 666 [M+H]⁺; ¹H-NMR (300 MHz, CD₃OD) δ 7.27 (dd, J=4.8 Hz, 2H), 6.99-7.04 (m, 2H), 6.50-6.68 (m, 2H), 4.32 (s, 2H), 3.87-3.94 (m, 3H), 3.55-3.75 (m, 3H), 3.27-3.36 (m, 3H), 2.89-3.06 (m, 5H), 2.60-2.63 (m, 2H), 2.24 (s, 1H), 1.39 (s, 2H), 1.14-1.17 (m, 2H), 0.64-0.66 (m, 2H), 0.17 (s, 2H).

### Example 81

### 1-cyclopropyl-4-[[(4R)-3-[(2,5-dichlorophenyl)methyl]-2,2-dimethyl-1,3-thiazolidin-4-yl]carbonyl]-1,2,3,4-tetrahydroquinoxaline

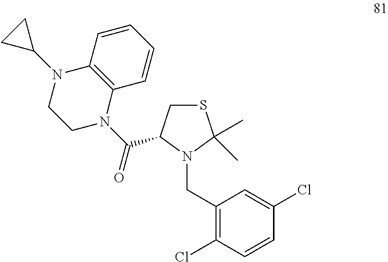 [SMILES: CC1(C)SC[C@@H](C(=O)N2CCN(C3CC3)c3ccccc32)N1Cc1cc(Cl)ccc1Cl]

Example 81 (S)-(4-cyclopropyl-3,4-dihydroquinoxaline-1(2H)-yl)(1-(2,5-dichlorobenzyl)-2-methylpyrrolidin-2-yl)methanone: Example 81 was prepared using the procedure described for the preparation of example 12, except that (4R)-2,2-dimethyl-1,3-thiazolidine-4-carboxylic acid was used in place of (S)-1-[(benzyloxy) carbonyl]pyrrolidine-2-carboxylic acid isolated as the bis TFA salt, a white solid. MS (ES, m/z): 476 [M+H]⁺. ¹H-NMR (300 MHz, CD₃OD) δ 7.39-7.20 (m, 2H), 7.17-7.11 (m, 4H), 6.66 (br s, 1H), 4.21 (s, 1H), 3.51 (br s, 1H), 3.28 (br s, 1H), 3.04 (br s, 2H), 2.25 (s, 1H), 2.00-1.26 (m, 6H), 0.71 (br s, 2H), 0.34-0.07 (br s, 2H).

### Example 82

### (2S)—N-(2-cyclopropoxyphenyl)-1-[(2,5-dichlorophenyl)methyl]-N-methylpyrrolidine-2-carboxamide

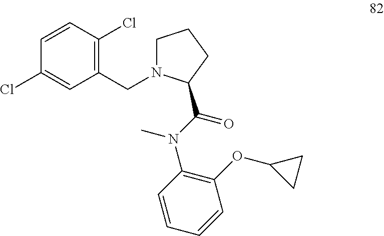 [SMILES: CN(C(=O)[C@@H]1CCCN1Cc1cc(Cl)ccc1Cl)c1ccccc1OC1CC1]

Example 82 (2S)—N-(2-cyclopropoxyphenyl)-1-[(2,5-dichlorophenyl)methyl]-N-methylpyrrolidine-2-carboxamide: Example 82 was prepared as described for example 12 substituting 2-cyclopropoxy-N-methylaniline 73g for 1-cyclopropyl-1,2,3,4-tetrahydro-quinoxaline. This resulted in 24 mg (33%) of the title compound trifluoroacetate salt as an off-white solid. LC-MS-(ES, m/z): 467 [M+H]⁺; ¹H-NMR (300 MHz, CD₃OD) δ 7.31 (d, J=7.8 Hz, 1H), 7.09 (s, 1H), 7.05 (d, J=3.6 Hz, 1H), 6.71-6.66 (m, 3H), 4.33 (s, 2H), 4.39-4.86 (m, 2H), 3.36-3.34 (m, 2H), 2.31-2.24 (m, 1H), 1.41-1.38 (m, 2H), 1.21-1.11 (m, 2H).

### Example 83

### 3-(2,5-dichloro-4-(((1-(4-cyclopropyl-1,2,3,4-tetrahydroquinoxaline-1-carbonyl)cyclopropyl)amino)methyl)phenyl)propanoic acid

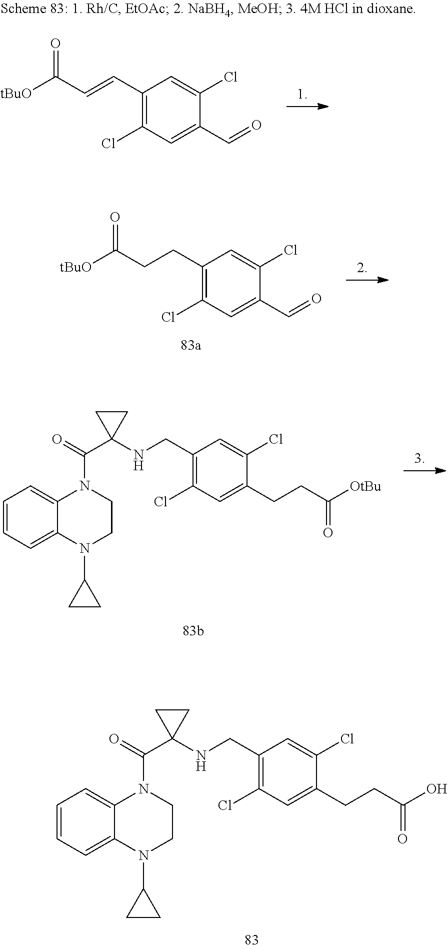 [SMILES: CC(C)(C)OC(=O)/C=C/c1cc(Cl)c(C=O)cc1Cl ; CC(C)(C)OC(=O)CCc1cc(Cl)c(C=O)cc1Cl ; CC(C)(C)OC(=O)CCc1cc(Cl)c(CNC2(C(=O)N3CCN(C4CC4)c4ccccc43)CC2)cc1Cl ; O=C(O)CCc1cc(Cl)c(CNC2(C(=O)N3CCN(C4CC4)c4ccccc43)CC2)cc1Cl]

Intermediate 83a: tert-butyl 3-(2,5-dichloro-4-formylphenyl)propanoate: Hydrogen gas was introduced to a stirred solution of tert-butyl(2E)-3-(2,5-dichloro-4-formylphenyl)prop-2-enoate (3 g, 9.96 mmol, 1.00 equiv) and 30% Rh/C (1.0g) in ethylacetate (30 mL). The resulting solution was stirred for 5 h at room temperature under a hydrogen atmosphere then the solids were removed by filtration and the filtrate was concentrated under reduced pressure. The resulting residue was purified by silica gel column chromatography using petroleum ether/ethyl acetate (30:1) as the eluent to provide 2.5 g (83%) of 83a as a colorless solid. MS (ES, m/z): (400 MHz, DMSOd₆): δ 10.21 (s, 1H), 7.81 (s, 1H), 7.63 (s, 1H), 2.92-2.99 (m, 2H), 2.55-2.62 (m, 2H), 1.36 (s, 9H).

Intermediate 83b: t-Butyl 3-(2,5-dichloro-4-(((1-(4-cyclopropyl-1,2,3,4-tetrahydroquinoxaline-1-carbonyl)cyclopropyl)amino)methyl)phenyl)propanoate. A mixture of (1-aminocyclopropyl)(4-cyclopropyl-3,4-dihydroquinoxalin-1-(2H)-yl)methanone free base (10.8 mg, 0.04 mmol), prepared form 26a by treating it with 4 M hydrochloric acid in 1,4-dioxane and then washed with saturated aqueous NaHCO₃, and t-butyl 3-(2,5-dichloro-4-formylphenyl)propanoate (12.5 mg, 0.04 mmol) in methanol (0.16 mL) was stirred at rt for 1 h. The mixture was cooled to 0° C. and to the mixture was added NaBH₄ (3.2 mg, 0.08 mmol). The resulting mixture was stirred at 0° C. for 15 minutes and at room temperature for 5 minutes. The addition of NaBH₄ was repeated three more times. The reaction mixture was quenched with 1M aqueous NaOH, extracted with EtOAc (3×). The combined organic layers were washed with brine (1×), dried over sodium sulfate, concentrated and purified by column to give 18 mg of 83b as yellow syrup.

Example 83 3-(2,5-Dichloro-4-(((1-(4-cyclopropyl-1,2,3,4-tetrahydroquinoxaline-1-carbonyl)cyclopropyl)amino)methyl)phenyl)propanoic acid: To t-butyl 3-(2,5-dichloro-4-(((1-(4-cyclopropyl-1,2,3,4-tetrahydroquinoxaline-1-carbonyl)cyclopropyl)amino)methyl)phenyl) propanoate (21 mg, 0.039 mmol) was added 4 M hydrochloric acid in dioxane (2 mL). The mixture was stirred at room temperature for 2 h and concentrated to give 14 mg (crude) of 3-(2,5-dichloro-4-(((1-(4-cyclopropyl-1,2,3,4-tetrahydroquinoxaline-1-carbonyl)cyclopropyl)amino)methyl)phenyl)propanoic acid as a solid. Some of the solid (4.7 mg) was purified by pre-HPLC to give 2.5 mg of 3-(2,5-dichloro-4-(((1-(4-cyclopropyl-1,2,3,4-tetrahydroquinoxaline-1-carbonyl)cyclopropyl)-amino)methyl)phenyl)propanoic acid TFA salt as a yellow solid. ¹H-NMR (400 MHz, CD₃OD) δ 7.37 (s, 1H), 7.29 (dd, J=7.9, 1.5 Hz, 1H), 7.23 (dd, J=8.3, 1.4 Hz, 1H), 7.15 (ddd, J=8.4, 7.3, 1.5 Hz, 1H), 7.10 (s, 1H), 6.78-6.72 (m, 1H), 3.94-3.83 (m, 4H), 3.44 (t, J=5.8 Hz, 2H), 2.97 (t, J=7.6 Hz, 2H), 2.58 (t, J=7.6 Hz, 2H), 2.45-2.37 (m, 1H), 1.38 (q, J=5.0 Hz, 2H), 1.11 (dd, J=7.6, 5.0 Hz, 2H), 0.86-0.77 (m, 2H), 0.54-0.45 (m, 2H). MS (ES, m/z): 488 [M+H]⁺.

### Example 84

### 1-[[(2S)-1-[[2-chloro-5-(trifluoromethyl)phenyl]methyl]pyrrolidin-2-yl]carbonyl]-4-cyclopropyl-1,2,3,4-tetrahydroquinoxaline

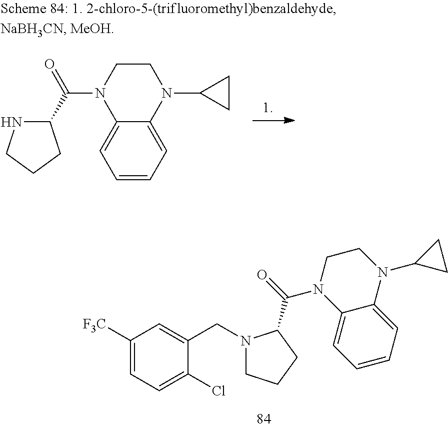 [SMILES: O=C([C@@H]1CCCN1)N1CCN(C2CC2)c2ccccc21 ; O=C([C@@H]1CCCN1Cc1cc(C(F)(F)F)ccc1Cl)N1CCN(C2CC2)c2ccccc21]

Example 84 1-[[(2S)-1-[[2-chloro-5-(trifluoromethyl)phenyl]methyl]-pyrrolidin-2-yl]carbonyl]-4-cyclopropyl-1,2,3,4-tetrahydroquinoxaline: To a stirred solution of 1-cyclopropyl-4-[[(2S)-pyrrolidin-2-yl]carbonyl]-1,2,3,4-tetrahydroquinoxaline (50 mg, 0.18 mmol, 1.00 equiv), 2-chloro-5-(trifluoromethyl)benzaldehyde (50 mg, 0.24 mmol, 1.30 equiv) in dichloromethane (4 mL) was added NaBH₃CN (50 mg, 0.80 mmol, 4.32 equiv). The resulting reaction mixture was stirred overnight at room temperature then quenched by the addition of 10 mL of water. The resulting solution was extracted with ethyl acetate (3×10 mL) and the combined organic layers washed with brine (10 mL), dried over anhydrous sodium sulfate and concentrated under reduced pressure. The crude residue (60 mg) was purified by preparative HPLC with the following conditions: Column, SunFire preparative C18, 19*150 mm 5 μm; mobile phase gradient, water containing 0.05% TFA:CH₃CN (30.0% CH₃CN up to 47.0% in 0 min); Detector, Waters 2545 UV detector at 254 and 220 nm to provide 11.1 mg (13%) of the title compound bis-trifluoroacetate salt as a yellow solid. MS (ES, m/z): 463 [M+H]⁺; ¹H-NMR (300 MHz, CD₃OD) δ 8.03 (s, 1H), 7.68-7.80 (m, 2H), 7.05-7.23 (m, 3H), 6.56-6.81 (m, 1H), 4.57-4.92 (m, 3H), 3.96-4.08 (m, 1H), 3.13-3.63 (m, 6H), 2.06-2.45 (m, 5H), 0.51-0.87 (m, 4H).

### Example 85

### 1-([1-[(2,5-dichlorophenyl)methoxy]cyclopropyl]carbonyl)-1,2,3,4-tetrahydro-1,8-naphthyridine

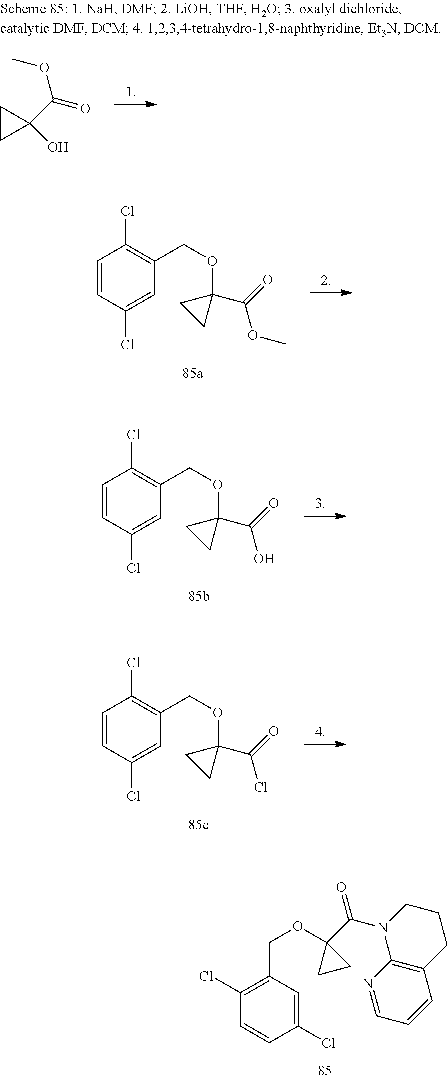

Intermediate 85a Methyl 1-[(2,5-dichlorophenyl)methoxy]cyclopropane-1-carboxylate: To a stirred 0° C. solution of methyl 1-hydroxycyclopropane-1-carboxylate (116 mg, 1.00 mmol, 1.00 equiv) in DMF (4 mL) was added sodium hydride (60 mg, 1.50 mmol, 1.50 equiv, 60% in mineral oil) in several batches. The resulting reaction mixture was stirred for 0.5 h at 0° C., then 2-(bromomethyl)-1,4-dichlorobenzene (238 mg, 0.99 mmol, 0.99 equiv) was added. The resulting reaction mixture was stirred for 1 h at room temperature and quenched by the addition of water (20 mL). The resulting solution was extracted with ethyl acetate (3×20 mL) and the combined organic layers washed with brine (20 mL), dried over anhydrous sodium sulfate, and concentrated under reduced pressure to provide 270 mg (98%) of 85a as a yellow oil.

Intermediate 85b 1-[(2,5-dichlorophenyl)methoxy]cyclopropane-1-carboxylic acid: To a stirred solution of methyl 1-[(2,5-dichlorophenyl)methoxy]cyclopropane-1-carboxylate (270 mg, 0.98 mmol, 1.00 equiv) in tetrahydrofuran (5 mL) and H₂O (2 mL) was added LiOH (240 mg, 10.02 mmol, 10.21 equiv), in portions. The resulting solution was stirred overnight at 30° C. in an oil bath. The pH value of the reaction mixture was adjusted to 5-6 with hydrogen chloride (2.0 M) then extracted with ethyl acetate (3×20 mL) and the combined organic layers dried over anhydrous sodium sulfate and concentrated under reduced pressure to provide 250 mg (98%) of 85b as a white solid.

Intermediate 85c 1-[(2,5-dichlorophenyl)methoxy]cyclopropane-1-carbonyl chloride: To a stirred solution of 1-[(2,5-dichlorophenyl)methoxy]cyclopropane-1-carboxylic acid (100 mg, 0.38 mmol, 1.00 equiv) in dichloromethane (4 mL) containing a catalytic amount of DMF was added oxalic dichloride (145 mg, 1.14 mmol, 3.00 equiv) dropwise. The resulting reaction mixture was stirred for 1 h at room temperature was concentrated under reduced pressure to provide 100 mg (93%) of 85c as a yellow solid.

Example 85 1-([1-[(2,5-dichlorophenyl)methoxy]cyclopropyl]carbonyl)-1,2,3,4-tetrahydro-1,8-naphthyridine: To a stirred solution of 1-[(2,5-dichlorophenyl)methoxy]cyclopropane-1-carbonyl chloride (100 mg, 0.36 mmol, 1.00 equiv), 1,2,3,4-tetrahydro-1,8-naphthyridine (51 mg, 0.38 mmol, 1.06 equiv), and dichloromethane (4 mL) was added triethylamine (77 mg, 0.76 mmol, 2.13 equiv) dropwise with stirring. The resulting reaction mixture was stirred for 3 h at room temperature, then quenched by the addition of water (10 mL), extracted with dichloromethane (3×20 mL). The combined organic layers were dried over anhydrous sodium sulfate and concentrated under reduced pressure. The crude residue (100 mg) was purified by preparative HPLC with the following conditions: Column, SunFire preparative C18, 19*150 mm 5 μm; mobile phase gradient, water containing 0.05% TFA: CH₃CN (38.0% CH₃CN to 56.0% over 6 min); Detector, Waters 2545 UV detector at 254 and 220 nm to provide 18.2 mg (13%) of the title compound trifluoroacetate salt as a yellow solid. MS (ES, m/z): 377 [M+H]⁺; ¹H-NMR (300 MHz, CD₃OD) δ 8.26-8.24 (m, 1H), 7.95-7.92 (m, 1H), 7.43-7.38 (m, 1H), 7.28-7.25 (m, 1H), 7.18-7.14 (m, 1H), 7.07 (s, 1H), 4.59 (s, 2H), 4.15 (t, J=6.0 Hz, 2H), 2.82 (t, J=6.3 Hz, 2H), 2.04-1.96 (m, 2H), 1.50-1.39 (m, 2H), 1.32-1.29 (m, 2H).

### Example 86

### 1-([1-[(2,5-dichlorophenyl)methoxy]cyclopropyl]carbonyl)-1,2,3,4-tetrahydro-1,5-naphthyridine

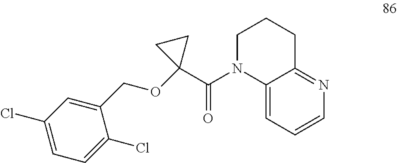 [SMILES: O=C(N1CCCc2ncccc21)C1(OCc2cc(Cl)ccc2Cl)CC1]

Example 86 1-([1-[(2,5-dichlorophenyl)methoxy]cyclopropyl]carbonyl)-1,2,3,4-tetrahydro-1,5-naphthyridine: Example 86 was prepared as described for example 85 substituting 1,2,3,4-tetrahydro-1,5-naphthyridine for 1,2,3,4-tetrahydro-1,8-naphthyridine to provide 47.2 mg (33%) of the title compound trifluoroacetate salt as a white solid. MS (ES, m/z): 377 [M+H]⁺; ¹H-NMR (300 MHz, CD₃OD) δ 8.51-8.54 (m, 1H), 8.35-8.38 (m, 1H), 7.65-7.70 (m, 1H), 7.17-7.32 (m, 3H), 4.60 (s, 2H), 4.07-4.11 (m, 2H), 3.05 (t, J=6.9 Hz, 2H), 2.04-2.12 (m, 2H), 1.30-1.45 (m, 4H).

### Example 87

### 3-(2,5-dichloro-4-[[(2S)-2-[(4-cyclopropyl-1,2,3,4-tetrahydroquinoxalin-1-yl)carbonyl]pyrrolidin-1-yl]methyl]phenyl)-N-[(2S,3R,4R,5R)-2,3,4,5,6-pentahydroxyhexyl]propanamide

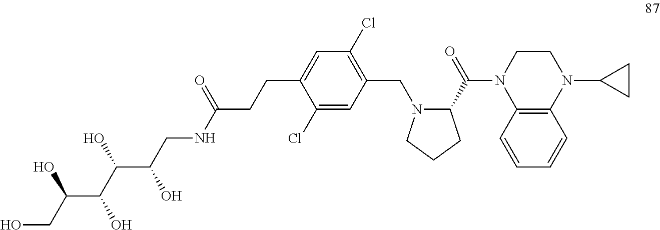 [SMILES: O=C(CCc1cc(Cl)c(CN2CCC[C@H]2C(=O)N2CCN(C3CC3)c3ccccc32)cc1Cl)NC[C@H](O)[C@@H](O)[C@H](O)[C@H](O)CO]

Example 87 3-(2,5-dichloro-4-[[(2S)-2-[(4-cyclopropyl-1,2,3,4-tetrahydroquinoxalin-1-yl)carbonyl]pyrrolidin-1-yl]methyl]phenyl)-N-[(2S,3R,4R,5R)-2,3,4,5,6-pentahydroxyhexyl]propanamide: Example 87 was prepared as described for example 48, substituting (2R,3R,4R,5S)-6-aminohexane-1,2,3,4,5-pentol for (2R,3R,4R,5S)-6-(methylamino)hexane-1,2,3,4,5-pentaol to provide 30 mg (23%) of the title compound bis-trifluoroacetate salt as a white solid. MS (ES, m/z): 665 [M+H]⁺; ¹H-NMR (300 MHz, CD₃OD) δ 7.52 (s, 1H), 7.27 (s, 2H), 7.11 (d, J=7.5 Hz, 1H), 5.50 (s, 1H), 4.58 (m, 2H), 4.23 (m, 1H), 3.65 (m, 4H), 3.42 (m, 2H), 3.15 (d, J=7.2 Hz, 2H), 2.60 (m, 3H), 2.07 (m, 3H), 0.88 (m, 2H), 0.67 (m, 2H).

### Example 88

### 3-[2,5-dichloro-4-([1-[(4-cyclopropyl-1,2,3,4-tetrahydroquinoxalin-1-yl)carbonyl]cyclopropoxy]methyl)phenyl]-N-[(2S,3R,4R,5R)-2,3,4,5,6-pentahydroxyhexyl]propanamide

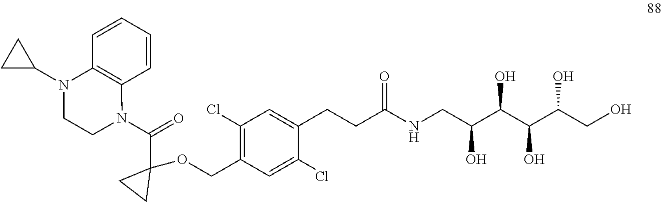 [SMILES: O=C(CCc1cc(Cl)c(COC2(C(=O)N3CCN(C4CC4)c4ccccc43)CC2)cc1Cl)NC[C@H](O)[C@@H](O)[C@H](O)[C@H](O)CO]

Example 88 3-[2,5-dichloro-4-([1-[(4-cyclopropyl-1,2,3,4-tetrahydroquinoxalin-1-yl)carbonyl]cyclopropoxy]methyl)phenyl]-N-[(2S,3R,4R,5R)-2,3,4,5,6-pentahydroxyhexyl]propanamide: Example 88 was prepared as described for example 80, substituting (2R,3R,4R,5S)-6-aminohexane-1,2,3,4,5-pentol for (2R,3R,4R,5S)-6-(methylamino)hexane-1,2,3,4,5-pentaol to provide 84.6 mg (40%) of the title compound trifluoroacetate salt as a pink solid. MS (ES, m/z): 652 [M+H]⁺; ¹H-NMR (300 MHz, CD₃OD) δ 7.27-7.32 (m, 2H), 7.01-7.07 (m, 2H), 6.72 (t, J=8.4 Hz, 2H), 4.36 (s, 2H), 3.92 (t, J=5.4 Hz, 2H), 3.32-3.81 (m, 9H), 2.96 (t, J=7.5 Hz, 2H), 2.46-2.51 (m, 2H), 2.27 (t, J=3.3 Hz, 1H), 1.43 (s, 2H), 1.19 (dd, J=7.5 Hz, 2H), 0.68 (t, J=8.1 Hz, 2H), 0.20 (s, 2H).

### Example 89

### (S)-(4-cyclopropyl-3,4-dihydroquinoxalin-1(2H)-yl)(1-(2,5-dichloro-4-methoxybenzyl)pyrrolidin-2-yl)methanone

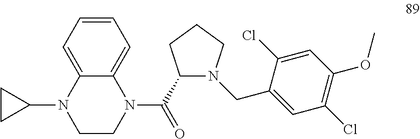 [SMILES: COc1cc(Cl)c(CN2CCC[C@H]2C(=O)N2CCN(C3CC3)c3ccccc32)cc1Cl]

Example 89 (4-cyclopropyl-3,4-dihydroquinoxalin-1(2H)-yl)(1-(1-(2,5-dichlorophenyl)ethoxy)cyclopropyl)methanone: Example 89 was prepared as described for example 84 substituting 92a for 2-chloro-5-(trifluoromethyl)benzaldehyde to provide 89 as the bis-trifluoroacetate salt. MS (ES, m/z): 460 [M+H]⁺.

### Example 90

### (4-cyclopropyl-3,4-dihydroquinoxalin-1(2H)-yl)(3-(2,5-dichlorobenzylamino)oxetan-3-yl)methanone

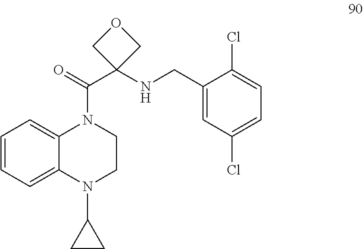 [SMILES: O=C(N1CCN(C2CC2)c2ccccc21)C1(NCc2cc(Cl)ccc2Cl)COC1]

Example 90 (4-cyclopropyl-3,4-dihydroquinoxalin-1(2H)-yl)(3-(2,5-dichlorobenzylamino)oxetan-3-yl)methanone: Example 90 was prepared as described for example 26 substituting 3-(tert-butoxycarbonylamino)oxetane-3-carboxylic acid for 1-(tert-butoxycarbonylamino)cyclopropanecarboxylic acid to provide 90. MS (ES, m/z): 432 [M+H]⁺; ¹H-NMR (400 MHz, CD₃OD) δ 7.48 (s, 2H), 7.36 (d, J=8.5 Hz, 1H), 7.25 (dd, J=8.5, 2.6 Hz, 1H), 7.18 (d, J=8.0 Hz, 1H), 7.08 (s, 1H), 6.67 (t, J=8.2 Hz, 1H), 5.08 (s, 2H), 4.66 (s, 2H), 3.95-3.59 (m, 4H), 3.39 (s, 2H), 2.48-2.32 (m, 1H), 0.87-0.74 (m, 2H), 0.50 (s, 2H).

### Example 91

### 3-(2,5-dichloro-4-((1-(4-cyclopropyl-1,2,3,4-tetrahydroquinoxaline-1-carbonyl)cyclopropylamino)methyl)phenyl)-N-methyl-N-((2S,3R,4R,5R)-2,3,4,5,6-pentahydroxyhexyl)propanamide

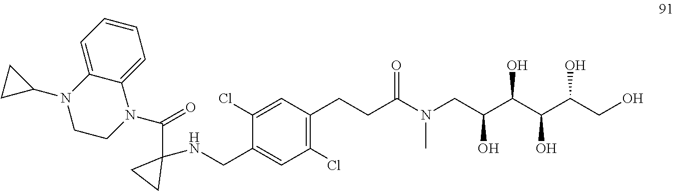 [SMILES: CN(C[C@H](O)[C@@H](O)[C@H](O)[C@H](O)CO)C(=O)CCc1cc(Cl)c(CNC2(C(=O)N3CCN(C4CC4)c4ccccc43)CC2)cc1Cl]

Example 91 (4-cyclopropyl-3,4-dihydroquinoxalin-1(2H)-yl)(3-(2,5-dichlorobenzylamino)oxetan-3-yl)methanone: Example 91 was prepared as described for example 80 substituting 83 for 80b to provide 91 as the TFA salt. ¹H-NMR (400 MHz, CD₃OD) δ 7.40 (d, J=2.7 Hz, 1H), 7.29 (dd, J=7.9, 1.4 Hz, 1H), 7.26-7.21 (m, 1H), 7.19-7.11 (m, 2H), 6.78-6.72 (m, 1H), 3.99-3.85 (m, 5H), 3.77 (dd, J=11.0, 3.4 Hz, 1H), 3.73-3.57 (m, 5H), 3.45 (t, J=5.8 Hz, 2H), 3.42-3.32 (m, 1H), 3.09 (s, 1.5H), 2.99 (t, J=7.8 Hz, 2H), 2.96 (s, 1.5H), 2.91-2.72 (m, 1H), 2.71-2.64 (m, 1H), 2.47-2.37 (m, 1H), 1.37 (q, J=5.1 Hz, 2H), 1.18-1.09 (m, 2H), 0.88-0.79 (m, 2H), 0.56-0.47 (m, 2H). MS (ES, m/z): 665 [M+H]⁺.

### Example 92

### (1-(4-(3-aminopropyl)-2,5-dichlorobenzylamino)cyclopropyl)(4-cyclopropyl-3,4-dihydroquinoxalin-1(2H)-yl)methanone

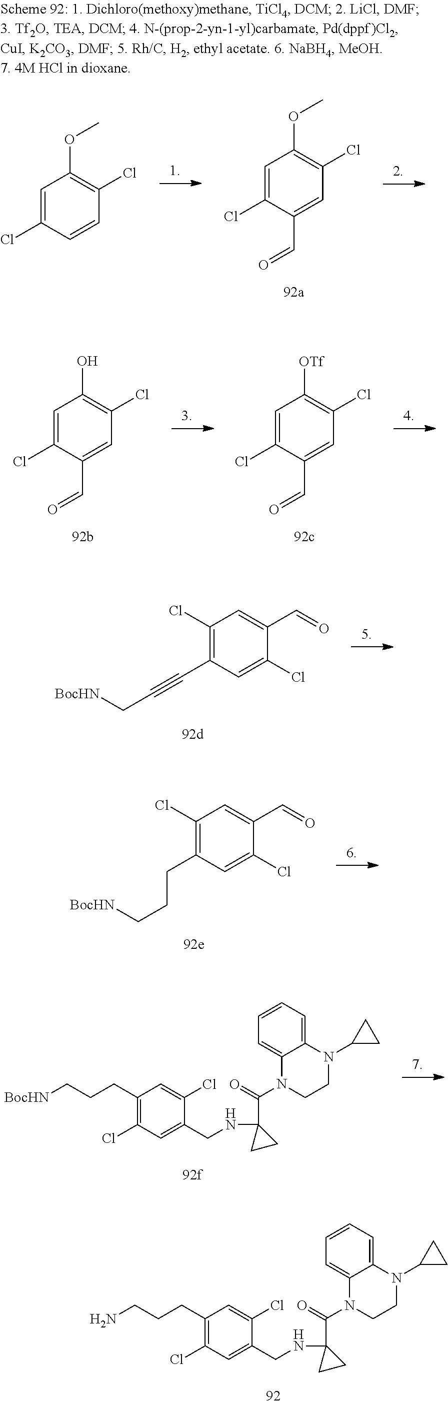

Intermediate 92a 2,5-dichloro-4-methoxybenzaldehyde: To a stirred 0° C. solution of 1,4-dichloro-2-methoxybenzene (25.0 g, 141.2 mmol, 1.00 equiv) and TiCl₄ (30.9 mL) in dichloromethane (300 mL) was added dichloro(methoxy)methane (16.2 g, 140.9 mmol, 1.00 equiv) dropwise. The resulting reaction mixture was stirred for 2 h at 60° C. then quenched by the addition of water/ice. The pH value of the solution was adjusted to 1.0 with concentrated HCl extracted with ethyl acetate (4×500 mL) and the combined organic layers washed with brine (2×500 mL), dried over anhydrous sodium sulfate and concentrated under reduced pressure to provide 31.0 g (crude) of 92a as a yellow solid.

Intermediate 92b 2,5-dichloro-4-hydroxybenzaldehyde: A solution of 2,5-dichloro-4-methoxybenzaldehyde (14.0 g, 68.3 mmol, 1.00 equiv), LiCl (11.6 g, 274 mmol, 4.00 equiv) in DMF (150 mL) under an inert atmosphere of nitrogen was stirred overnight at 140° C. in an oil bath. The reaction mixture was then quenched by the addition of water/ice and the pH value of the solution was adjusted to 1-2 with concentrated HCl. The resulting solution was extracted with ethylacetate (3×400 mL) and the combined organic layers were dried over anhydrous sodium sulfate and concentrated under reduced pressure. The resulting residue was purified using silica gel column chromatography with a ethyl acetate/petroleum ether (1:10-1:5) gradient to provide 10.0 g (77%) of 92b as a light yellow solid. (300 Hz, DMSOd₆): δ 11.99 (s, 1H), 10.08 (s, 1H), 7.81 (s, 1H), 7.09 (s, 1H).

Intermediate 92c 2,5-dichloro-4-formylphenyl trifluoromethanesulfonate: To a stirred 0° C. solution of 2,5-dichloro-4-hydroxybenzaldehyde (3.0 g, 15.71 mmol, 1.00 equiv) and triethylamine (3.2 g, 31.62 mmol, 2.00 equiv) in dichloromethane (50 mL) was added a solution of trifluoromethanesulfonic anhydride (6.8 g, 24.10 mmol, 1.50 equiv) in dichloromethane (10 mL) dropwise. The resulting reaction mixture was stirred for 30 min at room temperature then washed with brine (2×30 mL). The organic layer was dried over anhydrous sodium sulfate and concentrated under reduced pressure. Purification by silica gel column chromatography with an eluent gradient of ethyl acetate/petroleum ether (1:50-1:10) provided 3.0 g (59%) of 92c as a white solid. ¹H-NMR (300 Hz, DMSOd₆): 10.22 (s, 1H), 8.14-8.15 (m, 2H).

Intermediate 92d tert-butyl N-[3-(2,5-dichloro-4-formylphenyl)prop-2-yn-1-yl]carbamate: A solution of 2,5-dichloro-4-formylphenyl trifluoromethanesulfonate (5.0 g, 15.48 mmol, 1.00 equiv), tert-butyl N-(prop-2-yn-1-yl)carbamate (2.4 g, 15.46 mmol, 1.00 equiv), potassium carbonate (4.1 g, 29.7 mmol, 2.00 equiv), Pd(dppf)Cl₂ (1.2 g, 1.64 mmol, 0.10 equiv) and CuI (290 mg, 1.52 mmol, 0.10 equiv) in DMF (45.0 mL) was stirred overnight at room temperature under an inert N₂ atmosphere. The resulting reaction mixture was diluted with water (150 mL) extracted with ethyl acetate (3×150 mL) and the combined organic layers were washed with brine, dried over anhydrous sodium sulfate and concentrated under reduced pressure. Purification of the residue by silica gel column chromatography with an eluent gradient of ethyl acetate/petroleum ether (1:15-1:10) provided 2.0 g (39%) of 92d as a light yellow solid.

Intermediate 92e tert-butyl N-[3-(2,5-dichloro-4-formylphenyl)propyl]-carbamate: A solution of Rh/C (1.5 g), tert-butyl N-[3-(2,5-dichloro-4-formylphenyl)prop-2-yn-1-yl]carbamate (3.0 g, 9.14 mmol, 1.00 equiv) in ethyl acetate (45 mL) was stirred overnight under a hydrogen atmosphere at room temperature. Solids were removed from the reaction mixture and the filtrate was concentrated under reduced pressure. Purification of the resulting residue by silica gel column chromatography with an eluent gradient of ethyl acetate/petroleum ether (1:20-1:10) resulted in 2.4 g (79%) of 92e as a white solid. ¹H-NMR (300 Hz, DMSOd₆): 10.20 (s, 1H), 7.81 (s, 1H), 7.68 (s, 1H), 6.90-6.94 (m, 1H), 2.93-2.99 (m, 2H), 2.71-2.76 (m, 2H), 1.66-1.73 (m, 2H), 1.37 (s, 9H).

Example 92 3-(3-(2,5-dichloro-4-((1-(4-cyclopropyl-1,2,3,4-tetrahydro-quinoxaline-1-carbonyl)cyclopropylamino)methyl)phenyl)propyl)-1-methyl-1-((2S,3R,4R,5R)-2,3,4,5,6-pentahydroxyhexyl)urea: Example 92 was prepared as described for example 83 substituting tert-butyl N-[3-(2,5-dichloro-4-formylphenyl)propyl]carbamate 92e for 83a tert-butyl 3-(2,5-dichloro-4-formylphenyl)propanoate to provide 92 as the TFA salt. ¹H-NMR (400 MHz, CD₃OD) δ 7.34-7.29 (m, 2H), 7.22 (dd, J=8.3, 1.4 Hz, 1H), 7.16-7.10 (m, 1H), 6.99 (s, 1H), 6.74 (td, J=7.7, 1.4 Hz, 1H), 3.90 (t, J=5.8 Hz, 2H), 3.70 (s, 2H), 3.45 (t, J=5.8 Hz, 2H), 2.95 (t, J=8.0 Hz, 2H), 2.77 (t, J=8.0 Hz, 2H), 2.47-2.36 (m, 1H), 1.97-1.85 (m, 2H), 1.40 (q, J=4.5 Hz, 2H), 1.00 (q, J=4.5 Hz, 2H), 0.83-0.75 (m, 2H), 0.51-0.41 (m, 2H). MS (ES, m/z): 473 [M+H]⁺.

### Example 93

### 3-(3-(2,5-dichloro-4-((1-(4-cyclopropyl-1,2,3,4-tetrahydroquinoxaline-1-carbonyl)cyclopropylamino)methyl)phenyl)propyl)-1-methyl-1-((2S,3R,4R,5R)-2,3,4,5,6-pentahydroxyhexyl)urea

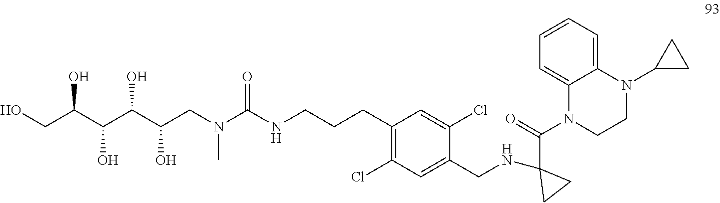 [SMILES: CN(C[C@H](O)[C@@H](O)[C@H](O)[C@H](O)CO)C(=O)NCCCc1cc(Cl)c(CNC2(C(=O)N3CCN(C4CC4)c4ccccc43)CC2)cc1Cl]

Example 93 3-(3-(2,5-dichloro-4-((1-(4-cyclopropyl-1,2,3,4-tetrahydro-quinoxaline-1-carbonyl)cyclopropylamino)methyl)phenyl)propyl)-1-methyl-1-((2S,3R,4R,5R)-2,3,4,5,6-pentahydroxyhexyl)urea: Example 93 was prepared as described for example 69 substituting 92 for 68 to provide 93 as the TFA salt. MS (ES, m/z): 694 [M+H]⁺; ¹H-NMR (400 MHz, CD₃OD) δ 7.36 (s, 1H), 7.29 (dd, J=7.9, 1.4 Hz, 1H), 7.23 (dd, J=8.3, 1.4 Hz, 1H), 7.19-7.12 (m, 1H), 7.10 (s, 1H), 6.75 (td, J=7.6, 1.4 Hz, 1H), 3.98-3.86 (m, 5H), 3.77 (dd, J=10.9, 3.2 Hz, 1H), 3.73-3.57 (m, 4H), 3.49-3.41 (m, 3H), 3.35 (d, J=8.0 Hz, 1H), 3.18 (t, J=6.9 Hz, 2H), 2.94 (s, 3H), 2.77-2.66 (m, 2H), 2.46-2.37 (m, 1H), 1.82-1.72 (m, 2H), 1.38 (q, J=5.0 Hz, 2H), 1.13 (q, J=5.1 Hz, 2H), 0.86-0.78 (m, 2H), 0.55-0.46 (m, 2H).

### Example 94

### (4-cyclopropyl-3,4-dihydroquinoxalin-1(2H)-yl)(1-(1-(2,5-dichlorophenyl)ethoxy)cyclopropyl)methanone

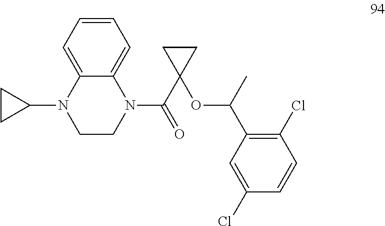 [SMILES: CC(OC1(C(=O)N2CCN(C3CC3)c3ccccc32)CC1)c1cc(Cl)ccc1Cl]

Example 94 (4-cyclopropyl-3,4-dihydroquinoxalin-1(2H)-yl)(1-(1-(2,5-dichlorophenyl)ethoxy)cyclopropyl)methanone was prepared as described for example 9 substituting 1-(2,5-dichlorophenyl)ethyl methanesulfonate for 2-(bromomethyl)-1,4-dichlorobenzene to provide 94 as the TFA salt. MS (ES, m/z): 431 [M+H]⁺.

### Example 95

### N,N′-(2,2′-(ethane-1,2-diylbis(oxy))bis(ethane-2,1-diyl))bis(3-(2,5-dichloro-4-((1-(4-cyclopropyl-1,2,3,4-tetrahydroquinoxaline-1-carbonyl)cyclopropylamino)methyl)phenyl)propanamide)

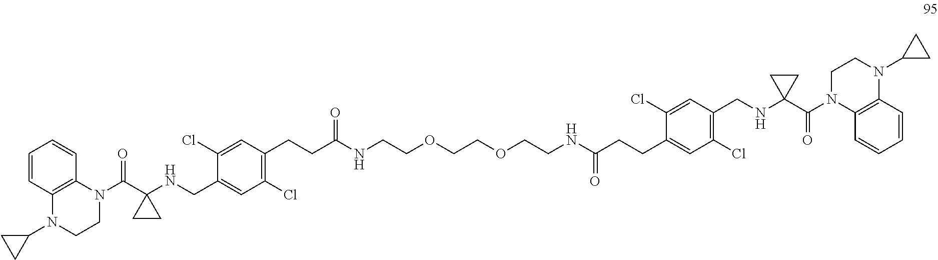 [SMILES: O=C(CCc1cc(Cl)c(CNC2(C(=O)N3CCN(C4CC4)c4ccccc43)CC2)cc1Cl)NCCOCCOCCNC(=O)CCc1cc(Cl)c(CNC2(C(=O)N3CCN(C4CC4)c4ccccc43)CC2)cc1Cl]

Example 95 N,N′-(2,2′-(ethane-1,2-diylbis(oxy))bis(ethane-2,1-diyl))bis(3-(2,5-dichloro-4-((1-(4-cyclopropyl-1,2,3,4-tetrahydroquinoxaline-1-carbonyl)cyclopropyl-amino)methyl)-phenyl)propanamide): Example 95 was prepared as described for example 91 using example 83 as the starting material and one half of an equivalent of 2,2′-(ethane-1,2-diylbis(oxy))-diethanamine in place of N-methyl-D-glucamine to provide 95 as the TFA salt. MS (ES, m/z): 1089 [M+I-1]⁺.

### Example 96

### N-(2-cyclopropoxyphenyl)-1-(2,5-dichloro-4-(3-(3-((2S,3R,4R,5R)-2,3,4,5,6-pentahydroxyhexyl)ureido)propyl)benzyloxy)-N-methylcyclopropanecarboxamide

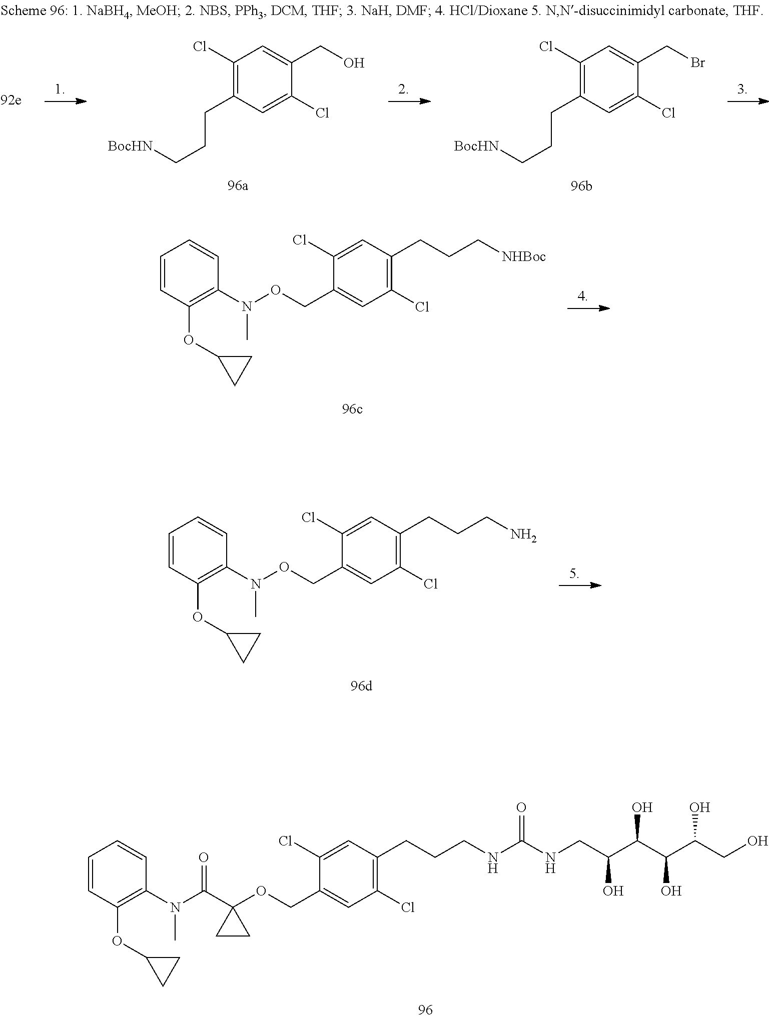 [SMILES: CC(C)(C)OC(=O)NCCCc1cc(Cl)c(CO)cc1Cl ; CC(C)(C)OC(=O)NCCCc1cc(Cl)c(CBr)cc1Cl ; CN(OCc1cc(Cl)c(CCCNC(=O)OC(C)(C)C)cc1Cl)c1ccccc1OC1CC1 ; CN(OCc1cc(Cl)c(CCCN)cc1Cl)c1ccccc1OC1CC1 ; CN(C(=O)C1(OCc2cc(Cl)c(CCCNC(=O)NC[C@H](O)[C@@H](O)[C@H](O)[C@H](O)CO)cc2Cl)CC1)c1ccccc1OC1CC1]

Intermediate 96d 1-(4-(3-aminopropyl)-2,5-dichlorobenzyloxy)-N-(2-cyclopropoxyphenyl)-N-methylcyclopropanecarboxamide: Intermediate 96d was prepared as described for intermediate 80b substituting N-(2-cyclopropoxyphenyl)-1-hydroxy-N-methylcyclopropane-1-carboxamide (73h) for 1-[(4-cyclopropyl-1,2,3,4-tetrahydroquinoxalin-1-yl)carbonyl]cyclopropan-1-ol in step 1 to provide intermediate 96d.

Example 96 N-(2-cyclopropoxyphenyl)-1-(2,5-dichloro-4-(3-(3-((2S,3R,4R,5R)-2,3,4,5,6-pentahydroxyhexyl)ureido)propyl)benzyloxy)-N-methylcyclopropanecarboxamide: Example 96 was prepared as described for example 69 substituting (2R,3R,4R,5S)-6-aminohexane-1,2,3,4,5-pentol for (2R,3R,4R,5 S)-6-(methylamino)hexane-1,2,3,4,5-pentaol to provide the title compound. MS (ES, m/z): 670 [M+H]⁺.

### Example 97

### 3-(2,5-dichloro-4-4(R)-4-(4-cyclopropyl-1,2,3,4-tetrahydroquinoxaline-1-carbonyl)thiazolidin-3-yl)methyl)phenyl)-N-((2S,3R,4R,5R)-2,3,4,5,6-pentahydroxyhexyl)propanamide

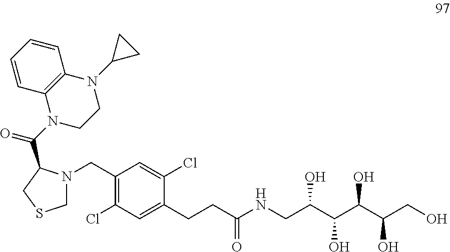 [SMILES: O=C(CCc1cc(Cl)c(CN2CSC[C@H]2C(=O)N2CCN(C3CC3)c3ccccc32)cc1Cl)NC[C@H](O)[C@@H](O)[C@@H](O)[C@H](O)CO]

Example 97 3-(2,5-dichloro-4-(((R)-4-(4-cyclopropyl-1,2,3,4-tetrahydroquinoxaline-1-carbonyl)thiazolidin-3-yl)methyl)phenyl)-N-((2S,3R,4R,5R)-2,3,4,5,6-pentahydroxyhexyl)propanamide: Example 97 was prepared as described for example 48 substituting (R)-(4-cyclopropyl-3,4-dihydroquinoxalin-1(2H)-yl)(thiazolidin-4-yl)methanone for (S)-(4-cyclopropyl-3,4-dihydroquinoxalin-1(2H)-yl)(pyrrolidin-2-yl)methanone in step 6 and (2R,3R,4R,5S)-6-aminohexane-1,2,3,4,5-pentol for (2R,3R,4R,5S)-6-(methylamino)hexane-1,2,3,4,5-pentol in step 8 to provide 97 as the bis TFA salt. MS (ES, m/z): 683 [M+H]⁺.

### Example 98

### 3-(2,5-dichloro-4-4(R)-4-(4-cyclopropyl-1,2,3,4-tetrahydroquinoxaline-1-carbonyl)thiazolidin-3-yl)methyl)phenyl)-N-methyl-N-((2S,3R,4S,5R)-2,3,4,5,6-pentahydroxyhexyl)propanamide

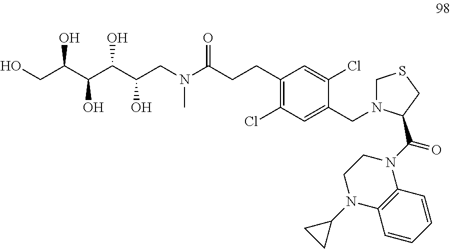 [SMILES: CN(C[C@H](O)[C@@H](O)[C@@H](O)[C@H](O)CO)C(=O)CCc1cc(Cl)c(CN2CSC[C@H]2C(=O)N2CCN(C3CC3)c3ccccc32)cc1Cl]

Example 98 3-(2,5-dichloro-4-(((R)-4-(4-cyclopropyl-1,2,3,4-tetrahydroquinoxaline-1-carbonyl)thiazolidin-3-yl)methyl)phenyl)-N-methyl-N-((2S,3R,4S,5R)-2,3,4,5,6-pentahydroxyhexyl)propanamide: Example 98 was prepared as described for example 48 substituting (R)-(4-cyclopropyl-3,4-dihydroquinoxalin-1(2H)-yl)(thiazolidin-4-yl)methanone for (S)-(4-cyclopropyl-3,4-dihydroquinoxalin-1(2H)-yl)(pyrrolidin-2-yl)methanone in step 6 and (2R,3S,4R,5S)-6-(methylamino)hexane-1,2,3,4,5-pentaol for (2R,3R,4R,5 S)-6-(methylamino)hexane-1,2,3,4,5-pentol in step 8 to provide 98 as the bis TFA salt. MS (ES, m/z): 697 [M+H]⁺.

### Examples 99-158

Compounds 99-158 are prepared from commercial or known starting materials according to the general methods described in Examples 1-98 and methods known to those skilled in the art.

| No. | Structure | Name |
|---|---|---|
| 99 | 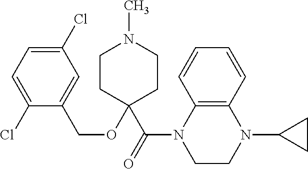 [SMILES: CN1CCC(OCc2cc(Cl)ccc2Cl)(C(=O)N2CCN(C3CC3)c3ccccc32)CC1] | 1-cyclopropyl-4-({4-[(2,5- dichlorophenyl)methoxy]- yl]-1-methylpiperidin-4- yl}carbonyl)-1,2,3,4- tetrahydroquinoxaline |
| 100 | 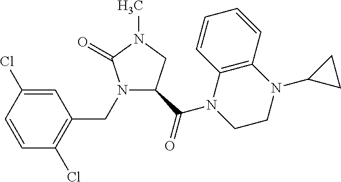 [SMILES: CN1C[C@@H](C(=O)N2CCN(C3CC3)c3ccccc32)N(Cc2cc(Cl)ccc2Cl)C1=O] | (4S)-4-[(4-cyclopropyl-1,2,3,4- tetrahydroquioxalin-1- yl)carbonyl]-3-[(2,5- dichlorophenyl)methyl]- 1-methylimidazolidin- 2-one |
| 101 | 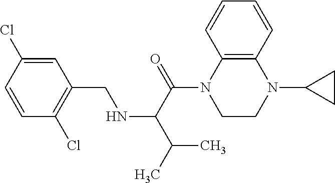 [SMILES: CC(C)C(NCc1cc(Cl)ccc1Cl)C(=O)N1CCN(C2CC2)c2ccccc21] | (2S)-1-(4-cyclopropyl-1,2,3,4- tetrahydroquinoxalin-1- yl)-2-{[(2,5- dichlorophenyl)methyl] amino}-3-methylbutan- 1-one |
| 102 | 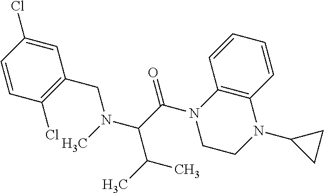 [SMILES: CC(C)C(C(=O)N1CCN(C2CC2)c2ccccc21)N(C)Cc1cc(Cl)ccc1Cl] | (2S)-1-(4-cyclopropyl-1,2,3,4- tetrahydroquinoxalin-1- yl)-2-{[(2,5- dichlorophenyl)methyl] (methyl)amino}-3- methylbutan-1-one |
| 103 | 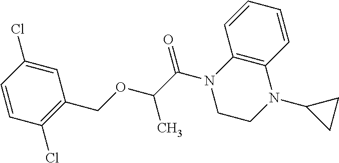 [SMILES: CC(OCc1cc(Cl)ccc1Cl)C(=O)N1CCN(C2CC2)c2ccccc21] | (2R)-1-(4-cycloproyl-1,2,3,4- tetrahydroquinoxalin-1- yl)-2-[(2,5- dichlorophenyl)methoxy] propan-1-one |
| 104 | 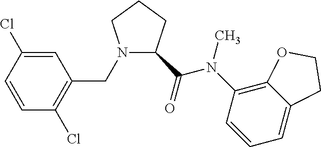 [SMILES: CN(C(=O)[C@@H]1CCCN1Cc1cc(Cl)ccc1Cl)c1cccc2c1OCC2] | (2S)-1-[(2,5- dichlorophenyl)methyl]- N-(2,3-dihydro-1- benzofuran-7-yl)-N- methylpyrrolidine-2- carboxamide |
| 105 | 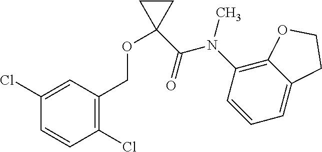 [SMILES: CN(C(=O)C1(OCc2cc(Cl)ccc2Cl)CC1)c1cccc2c1OCC2] | 1-[(2,5- dichlorophenyl)methoxy]- N-(2,3-dihydro-1- benzofuran-7-yl)-N- methylcyclopropane-1- carboxamide |
| 106 | 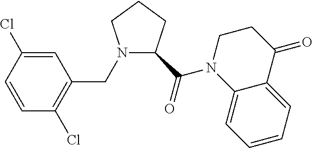 [SMILES: O=C1CCN(C(=O)[C@@H]2CCCN2Cc2cc(Cl)ccc2Cl)c2ccccc21] | 1-{[(2S)-1-[(2,5- dichlorophenyl)methyl] pyrrolidin-2- yl]carbonyl}-1,2,3,4- tetrahydroquinolin-4- one |
| 107 | 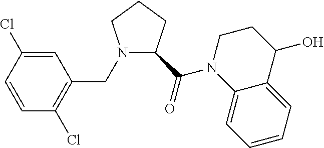 [SMILES: O=C([C@@H]1CCCN1Cc1cc(Cl)ccc1Cl)N1CCC(O)c2ccccc21] | 1-{[(2S)-1-[(2,5- dichlorophenyl)methyl] pyrrolidin-2- yl]carbonyl}-1,2,3,4- tetrahydroquinolin-4-ol |
| 108 | 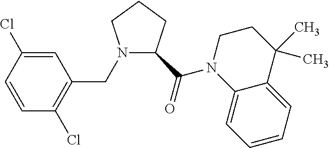 [SMILES: CC1(C)CCN(C(=O)[C@@H]2CCCN2Cc2cc(Cl)ccc2Cl)c2ccccc21] | 1-{[(2S)-1-[(2,5- dichlorophenyl)methyl] pyrrolidin-2- yl]carbonyl}-4-methyl-1,2,3,4- tetrahydroquinolin-4-ol |
| 109 | 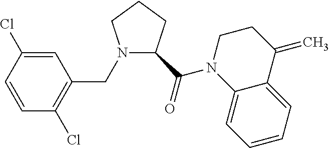 [SMILES: C=C1CCN(C(=O)[C@@H]2CCCN2Cc2cc(Cl)ccc2Cl)c2ccccc21] | 1-{[(2S)-1-[(2,5- dichlorophenyl)methyl] pyrrolidin-2-yl]carbonyl}-4- methylidene-1,2,3,4- tetrahydroquinoline |
| 110 | 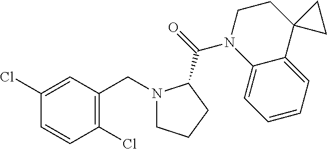 [SMILES: O=C([C@@H]1CCCN1Cc1cc(Cl)ccc1Cl)N1CCC2(CC2)c2ccccc21] | 1′-{[(2S)-1-[(2,5- dichlorophenyl)methyl] pyrrolidin-2- yl]carbonyl}-2′,3′- dihydro-1′H- spiro[cyclopropane- 1,4′-quinoline] |
| 111 | 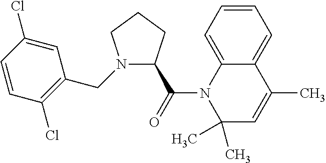 [SMILES: CC1=CC(C)(C)N(C(=O)[C@@H]2CCCN2Cc2cc(Cl)ccc2Cl)c2ccccc21] | 1-{[(2S)-1-[(2,5- dichlorophenyl)methyl] pyrrolidin-2- yl]carbonyl}-2,2,4- trimethyl-1,2- dihydroquinoline |
| 112 | 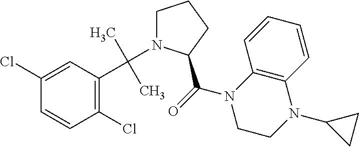 [SMILES: CC(C)(c1cc(Cl)ccc1Cl)N1CCC[C@H]1C(=O)N1CCN(C2CC2)c2ccccc21] | 1-cyclopropyl-4- {[(2S)-1-[2-(2,5- dichlorophenyl)propan- 2-yl]pyrrolidin-2- yl]carbonyl}-1,2,3,4- tetrahydroquinoxaline |
| 113 | 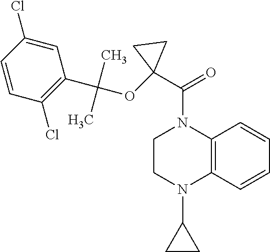 [SMILES: CC(C)(OC1(C(=O)N2CCN(C3CC3)c3ccccc32)CC1)c1cc(Cl)ccc1Cl] | 1-cyclopropyl-4-[(1-{[2-(2,5- dichlorophenyl)propan-2- yl]oxy}cyclopropyl)car- bonyl]-1,2,3,4- tetrahydroquinoxaline |
| 114 | 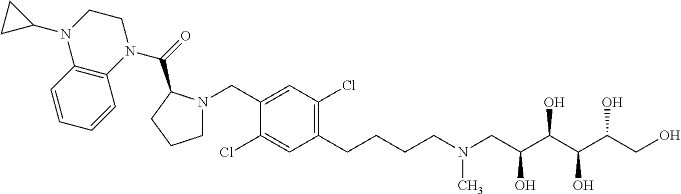 [SMILES: CN(CCCCc1cc(Cl)c(CN2CCC[C@H]2C(=O)N2CCN(C3CC3)c3ccccc32)cc1Cl)C[C@H](O)[C@@H](O)[C@H](O)[C@H](O)CO] | (2R,3R,4R,5S)-6-{[4- (2,5-dichloro-4-{[(2S)- 2-[(4-cyclopropyl-1,2,3,4- tetrahydroquinoxalin-1- yl)carbonyl]pyrrolidin-1- yl]methyl}phenyl)butyl] (methyl)amino}hexane- 1,2,3,4,5-pentol |
| 115 | 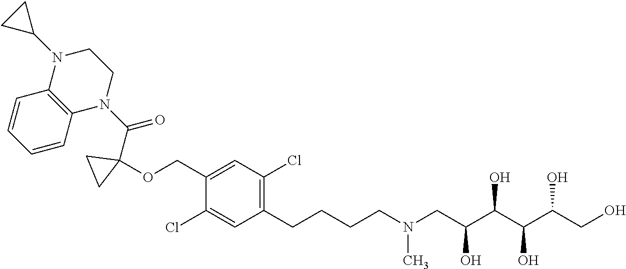 [SMILES: CN(CCCCc1cc(Cl)c(COC2(C(=O)N3CCN(C4CC4)c4ccccc43)CC2)cc1Cl)C[C@H](O)[C@@H](O)[C@H](O)[C@H](O)CO] | (2R,3R,4R,5S)-6-({4- [2,5-dichloro-4-({1-[(4- cyclopropyl-1,2,3,4- tetrahydroquinoxalin-1- yl)carbonyl]cyclopro- poxy}methyl)phenyl]butyl} (methyl)amino)hexane- 1,2,3,4,5-pentol |
| 116 | 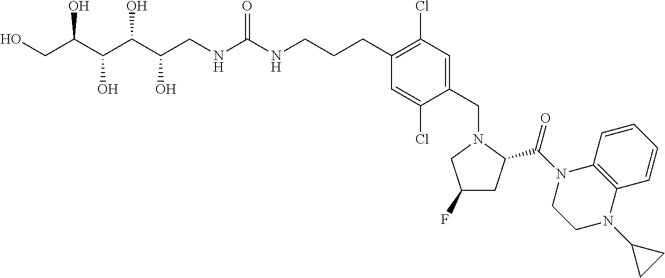 [SMILES: O=C(NCCCc1cc(Cl)c(CN2C[C@H](F)C[C@H]2C(=O)N2CCN(C3CC3)c3ccccc32)cc1Cl)NC[C@H](O)[C@@H](O)[C@H](O)[C@H](O)CO] | 1-[3-(2,5-dichloro-4- {[(2S)-2-[(4- cyclopropyl-1,2,3,4- tetrahydroquinoxalin-1- yl)carbonyl]pyrrolidin-1- yl]methyl}phenyl)pro- pyl]-3-[(2S,3R,4R,5R)- 2,3,4,5,6- pentahydroxyhexyl]urea |
| 117 | 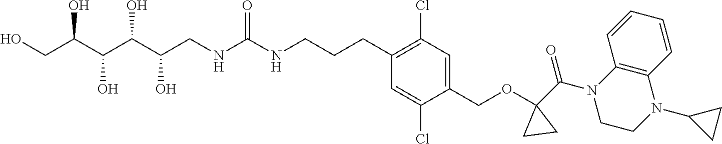 [SMILES: O=C(NCCCc1cc(Cl)c(COC2(C(=O)N3CCN(C4CC4)c4ccccc43)CC2)cc1Cl)NC[C@H](O)[C@@H](O)[C@H](O)[C@H](O)CO] | 1-{3-[2,5-dichloro-4- ({1-[(4-cyclopropyl-1,2,3,4- tetrahydroquinoxalin-1- yl)carbonyl]cyclopro- poxy}methyl)phenyl]pro- pyl}-3-[(2S,3R,4R,5R)- 2,3,4,5,6- pentahydroxyhexyl]urea |
| 118 | 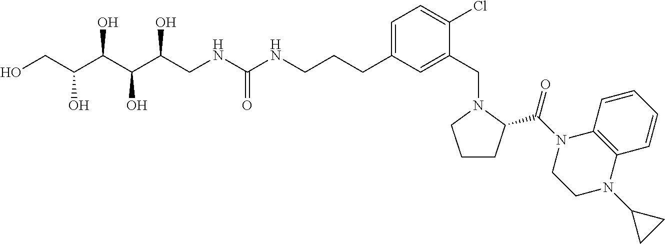 [SMILES: O=C(NCCCc1ccc(Cl)c(CN2CCC[C@H]2C(=O)N2CCN(C3CC3)c3ccccc32)c1)NC[C@H](O)[C@@H](O)[C@H](O)[C@H](O)CO] | 1-[3-(4-chloro-3- {[(2S)-2-[(4- cyclopropyl-1,2,3,4- tetrahydroquinoxalin-1- yl)carbonyl]pyrrolidin-1- yl]methyl}phenyl)pro- pyl]-3-[(2S,3R,4R,5R)- 2,3,4,5,6- pentahydroxyhexyl]urea |
| 119 | 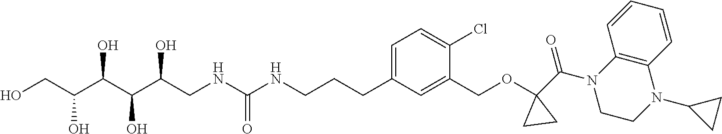 [SMILES: O=C(NCCCc1ccc(Cl)c(COC2(C(=O)N3CCN(C4CC4)c4ccccc43)CC2)c1)NC[C@H](O)[C@@H](O)[C@H](O)[C@H](O)CO] | 1-{3-[4-chloro-3-({1- [(4-cyclopropyl-1,2,3,4- tetrahydroquinoxalin-1- yl)carbonyl]cyclopro- poxy}methyl)phenyl]pro- pyl}-3-[(2S,3R,4R,5R)- 2,3,4,5,6- pentahydroxyhexyl]urea |
| 120 | 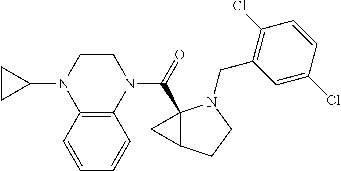 [SMILES: O=C(N1CCN(C2CC2)c2ccccc21)[C@]12CC1CCN2Cc1cc(Cl)ccc1Cl] | 1-cyclopropyl-4- {[(2S)-2-[(2,5- dichlorophenyl)methyl]-2- azabicyclo[3.1.0]hexan- 1-yl]carbonyl}-1,2,3,4- tetrahydroquinoxaline |
| 121 | 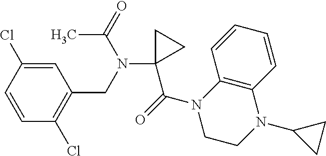 [SMILES: CC(=O)N(Cc1cc(Cl)ccc1Cl)C1(C(=O)N2CCN(C3CC3)c3ccccc32)CC1] | N-{1-[(4-cyclopropyl-1,2,3,4- tetrahydroquinoxalin-1- yl)carbonyl]cyclopropyl}- N-[(2,5-dichlorophenyl)methyl] acetamide |
| 122 | 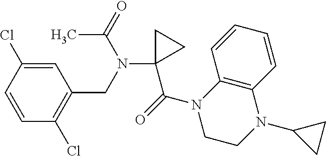 [SMILES: CC(=O)N(Cc1cc(Cl)ccc1Cl)C1(C(=O)N2CCN(C3CC3)c3ccccc32)CC1] | 1-{1-[(4-cyclopropyl-1,2,3,4- tetrahydroquinoxalin-1- yl)carbonyl]cyclopropyl}- 1-[(2,5-dichlorophenyl)methyl] urea |
| 123 | 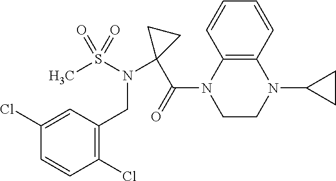 [SMILES: CS(=O)(=O)N(Cc1cc(Cl)ccc1Cl)C1(C(=O)N2CCN(C3CC3)c3ccccc32)CC1] | N-{1-[(4-cyclopropyl-1,2,3,4- tetrahydroquinoxalin-1- yl)carbonyl]cyclopropyl}- N-[(2,5-dichlorophenyl)methyl] methanesulfonamide |
| 124 | 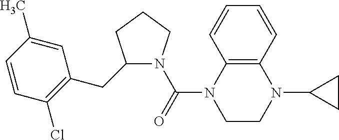 [SMILES: Cc1ccc(Cl)c(CC2CCCN2C(=O)N2CCN(C3CC3)c3ccccc32)c1] | 1-({2-[(2-chloro-5- methylphenyl)methyl] pyrrolidin-1-yl}carbonyl)-4- cyclopropyl-1,2,3,4- tetrahydroquinoxaline |
| 125 | 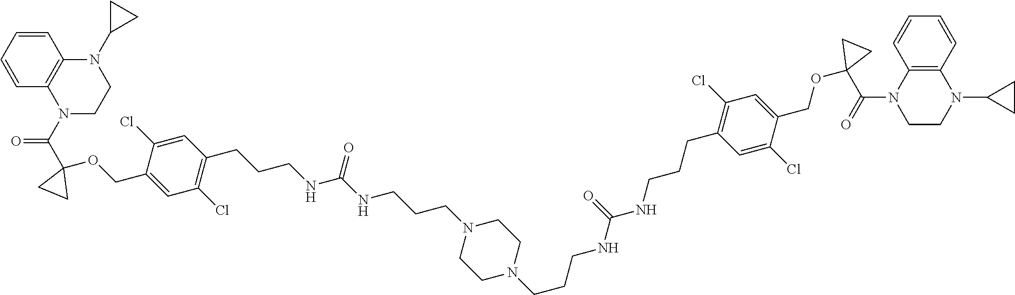 [SMILES: O=C(NCCCc1cc(Cl)c(COC2(C(=O)N3CCN(C4CC4)c4ccccc43)CC2)cc1Cl)NCCCN1CCN(CCCNC(=O)NCCCc2cc(Cl)c(COC3(C(=O)N4CCN(C5CC5)c5ccccc54)CC3)cc2Cl)CC1] | 3-{3-[2,5-dichloro-4- ({1-[(4-cyclopropyl-1,2,3,4- tetrahydroquinoxalin-1- yl)carbonyl]cyclopropoxy} methyl)phenyl]pro- pyl}-1-[3-(4-{3-[({3- [2,5-dichloro-4-({1-[(4- cyclopropyl-1,2,3,4- tetrahydroquinoxalin-1- yl)carbonyl]cyclopropoxy} methyl)phenyl]propyl} carbamoyl)amino] propyl}piperazin-1- yl)propyl]urea |
| 126 | 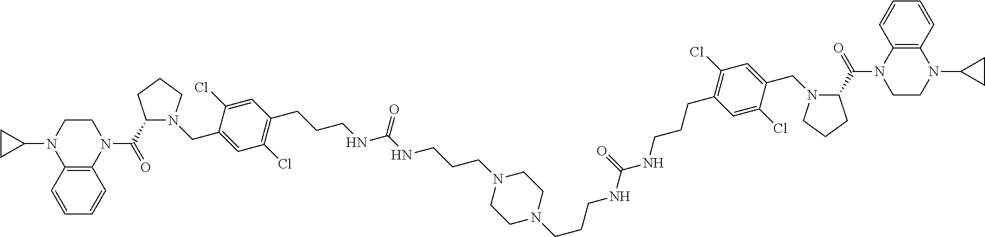 [SMILES: O=C(NCCCc1cc(Cl)c(CN2CCC[C@H]2C(=O)N2CCN(C3CC3)c3ccccc32)cc1Cl)NCCCN1CCN(CCCNC(=O)NCCCc2cc(Cl)c(CN3CCC[C@H]3C(=O)N3CCN(C4CC4)c4ccccc43)cc2Cl)CC1] | 3-[3-(2,5-dichloro-4- {[(2S)-2-[(4-cyclopropyl- 1,2,3,4- tetrahydroquinoxalin-1- yl)carbonyl]pyrrolidin-1- yl]methyl}phenyl)pro- pyl]-1-(3-{4-[3-({[3- (2,5-dichloro-4-{[(2S)- 2-[(4-cyclopropyl-1,2,3,4- tetrahydroquinoxalin-1- yl)carbonyl]pyrrolidin-1- yl]methyl}phenyl)pro- pyl]carbamoyl}amino)pro- pyl]piperazin-1-yl}propyl)urea |
| 127 | 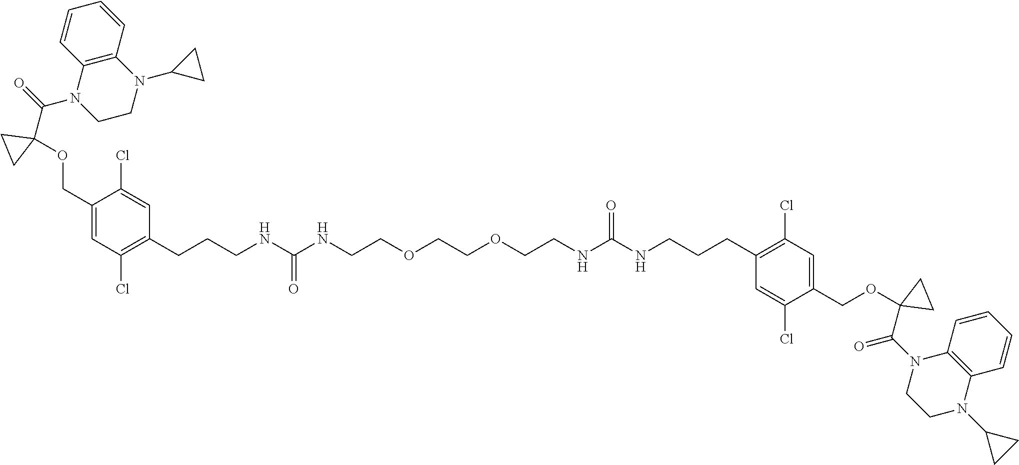 [SMILES: O=C(NCCCc1cc(Cl)c(COC2(C(=O)N3CCN(C4CC4)c4ccccc43)CC2)cc1Cl)NCCOCCOCCNC(=O)NCCCc1cc(Cl)c(COC2(C(=O)N3CCN(C4CC4)c4ccccc43)CC2)cc1Cl] | 3-{3-[2,5-dichloro-4- ({1-[(4-cyclopropyl-1,2,3,4- tetrahydroquinoxalin-1- yl)carbonyl]cyclopropoxy} methyl)phenyl]propyl}- 1-[2-(2-{2-[({3- [2,5-dichloro-4-({1-[(4- cyclopropyl-1,2,3,4- tetrahydroquinoxalin-1- yl)carbonyl]cyclopropoxy} methyl)phenyl]pro- pyl}carbamoyl)amino]eth- oxy}ethoxy)ethyl]urea |
| 128 | 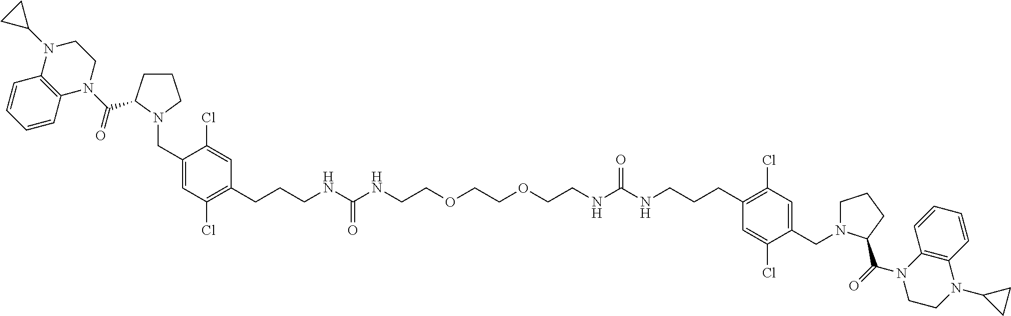 [SMILES: O=C(NCCCc1cc(Cl)c(CN2CCC[C@H]2C(=O)N2CCN(C3CC3)c3ccccc32)cc1Cl)NCCOCCOCCNC(=O)NCCCc1cc(Cl)c(CN2CCC[C@H]2C(=O)N2CCN(C3CC3)c3ccccc32)cc1Cl] | 3-[3-(2,5-dichloro-4- {[(2S)-2-[(4- cyclopropyl-1,2,3,4- tetrahydroquinoxalin-1- yl)carbonyl]pyrrolidin-1- yl]methyl}phenyl)pro- pyl]-1-(2-{2-[2-({[3- (2,5-dichloro-4-{[(2S)- 2-[(4-cyclopropyl-1,2,3,4- tetrahydroquinoxalin-1- yl)carbonyl]pyrrolidin-1- yl]methyl}phenyl)pro- pyl]carbamoyl}amino)eth- oxy]ethoxy}ethyl)urea |
| 129 | 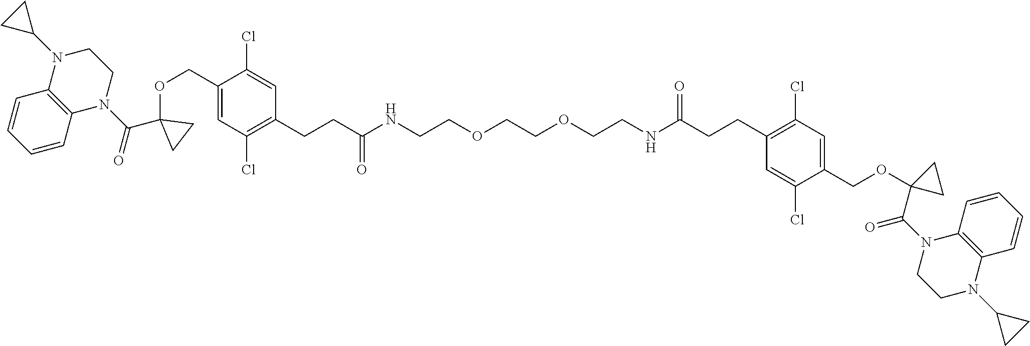 [SMILES: O=C(CCc1cc(Cl)c(COC2(C(=O)N3CCN(C4CC4)c4ccccc43)CC2)cc1Cl)NCCOCCOCCNC(=O)CCc1cc(Cl)c(COC2(C(=O)N3CCN(C4CC4)c4ccccc43)CC2)cc1Cl] | 3-[2,5-dichloro-4-({1- [(4-cyclopropyl-1,2,3,4- tetrahydroquinoxalin-1- yl)carbonyl]cyclopropoxy} methyl)phenyl]-N- {2-[2-(2-{3-[2,5- dichloro-4-({1-[(4- cyclopropyl-1,2,3,4- tetrahydroquinoxalin-1- yl)carbonyl]cyclopropoxy} methyl)phenyl]propana- mido}ethoxy)ethoxy] ethyl}propanamide |
| 130 | 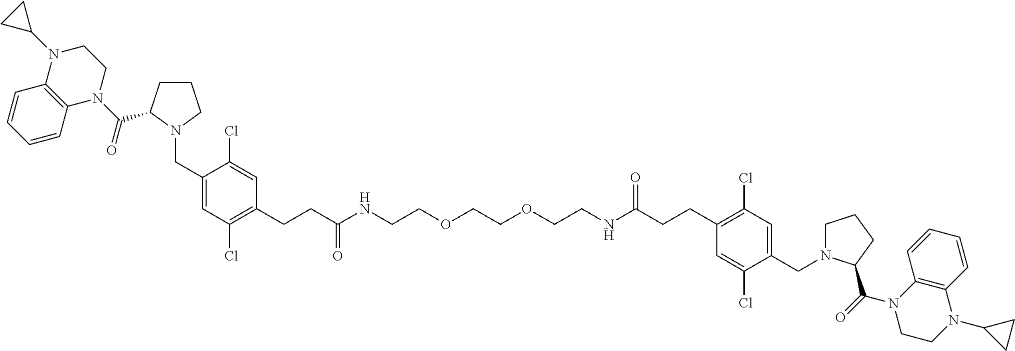 [SMILES: O=C(CCc1cc(Cl)c(CN2CCC[C@H]2C(=O)N2CCN(C3CC3)c3ccccc32)cc1Cl)NCCOCCOCCNC(=O)CCc1cc(Cl)c(CN2CCC[C@H]2C(=O)N2CCN(C3CC3)c3ccccc32)cc1Cl] | 3-(2,5-dichloro-4- {[(2S)-2-[(4- cyclopropyl-1,2,3,4- tetrahydroquinoxalin-1- yl)carbonyl]pyrrolidin- 1-yl]methyl}phenyl)- N-[2-(2-{2-[3-(2,5- dichloro-4-{[(2S)-2- [(4-cyclopropyl-1,2,3,4- tetrahydroquinoxalin-1- yl)carbonyl]pyrrolidin-1- yl]methyl}phenyl)propana- mido]ethoxy}ethoxy) ethyl]propanamide |
| 131 | 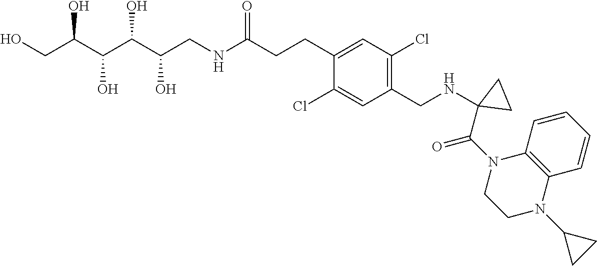 [SMILES: O=C(CCc1cc(Cl)c(CNC2(C(=O)N3CCN(C4CC4)c4ccccc43)CC2)cc1Cl)NC[C@H](O)[C@@H](O)[C@H](O)[C@H](O)CO] | 3-{2,5-dichloro-4-[({1- [(4-cyclopropyl-1,2,3,4- tetrahydroquinoxalin-1- yl)carbonyl]cyclopropyl} amino)methyl]phenyl}- N-[(2S,3R,4R,5R)- 2,3,4,5,6-pentahydroxyhexyl]- propanamide |
| 132 | 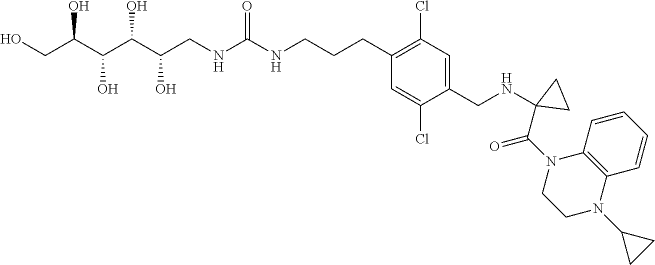 [SMILES: O=C(NCCCc1cc(Cl)c(CNC2(C(=O)N3CCN(C4CC4)c4ccccc43)CC2)cc1Cl)NC[C@H](O)[C@@H](O)[C@H](O)[C@H](O)CO] | 1-(3-{2,5-dichloro-4- [({1-[(4-cyclopropyl-1,2,3,4- tetrahydroquinoxalin-1- yl)carbonyl]cyclopropyl} amino)methyl]phenyl} propyl)-3-[(2S,3R,4R,R)- 2,3,4,5,6- pentahydroxyhexyl]urea |
| 133 | 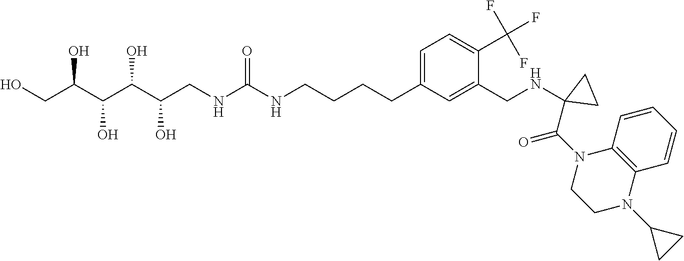 [SMILES: O=C(NCCCCc1ccc(C(F)(F)F)c(CNC2(C(=O)N3CCN(C4CC4)c4ccccc43)CC2)c1)NC[C@H](O)[C@@H](O)[C@H](O)[C@H](O)CO] | 1-(4-{3-[({1-[(4- cyclopropyl-1,2,3,4- tetrahydroquinoxalin-1- yl)carbonyl]cyclopropyl} amino)methyl]-4- (trifluoromethyl)phenyl} butyl)-3-[(2S,3R,4R,5R)- 2,3,4,5,6- pentahydroxyhexyl]urea |
| 134 | 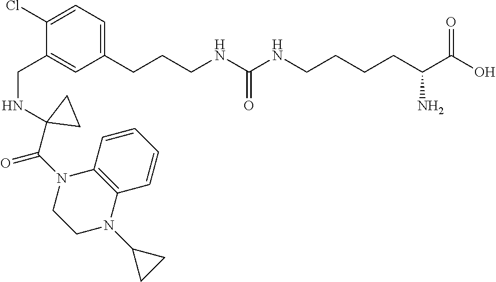 [SMILES: N[C@H](CCCCNC(=O)NCCCc1ccc(Cl)c(CNC2(C(=O)N3CCN(C4CC4)c4ccccc43)CC2)c1)C(=O)O] | (2R)-2-amino-6-{[(3- {4-chloro-3-[({1-[(4- cyclopropyl-1,2,3,4- tetrahydroquinoxalin-1- yl)carbonyl]cyclopropyl} amino)methyl]phenyl} propyl)carbamoyl]amino} hexanoic acid |
| 135 | 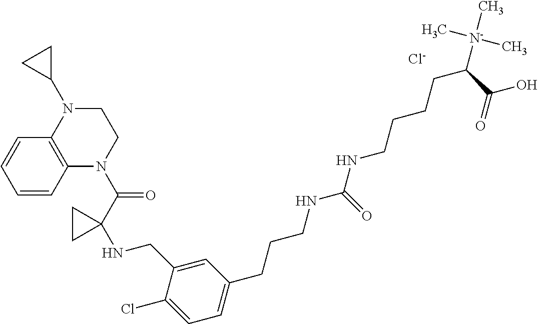 | [(1R)-1-carboxy-5- {[(3-{4-chloro-3-[({1- [(4-cyclopropyl-1,2,3,4- tetrahydroquinoxalin-1- yl)carbonyl]cyclopropyl} amino)methyl]phenyl} propyl)carbamoyl]ami- no}pentyl]trimethylaza- nium |
| 136 | 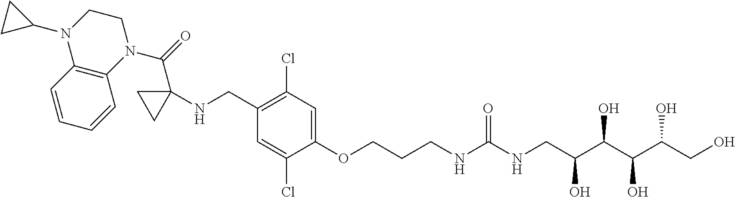 [SMILES: O=C(NCCCOc1cc(Cl)c(CNC2(C(=O)N3CCN(C4CC4)c4ccccc43)CC2)cc1Cl)NC[C@H](O)[C@@H](O)[C@H](O)[C@H](O)CO] | 1-(3-(2,5-dichloro-4- ((1-(4-cyclopropyl-1,2,3,4- tetrahydroquinoxaline-1- carbonyl)cyclopropyla- mino)methyl)phenoxy) propyl)-3-((2S,3R,4R,5R)- 2,3,4,5,6- pentahydroxyhexyl)urea |
| 137 | 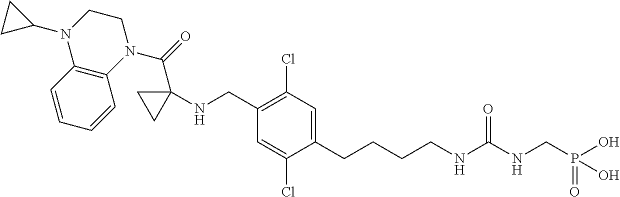 [SMILES: O=C(NCCCCc1cc(Cl)c(CNC2(C(=O)N3CCN(C4CC4)c4ccccc43)CC2)cc1Cl)NCP(=O)(O)O] | ({[(4-{2,5-dichloro-4- [({1-[(4-cyclopropyl-1,2,3,4- tetrahydroquinoxalin-1- yl)carbonyl]cyclopropyl} amino)methyl]phenyl} butyl)carbamoyl]amino} methyl)phosphonic acid |
| 138 | 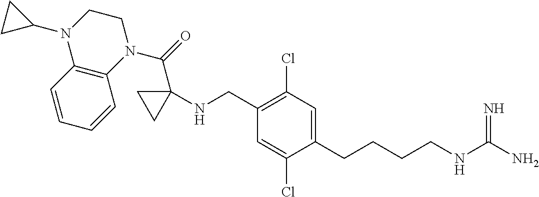 [SMILES: N=C(N)NCCCCc1cc(Cl)c(CNC2(C(=O)N3CCN(C4CC4)c4ccccc43)CC2)cc1Cl] | 1-{4-[2,5-dichloro-4- ({1-[(4-cyclopropyl-1,2,3,4- tetrahydroquinoxalin-1- yl)carbonyl]cyclopropoxy} methyl)phenyl]butyl} guanidine |
| 139 | 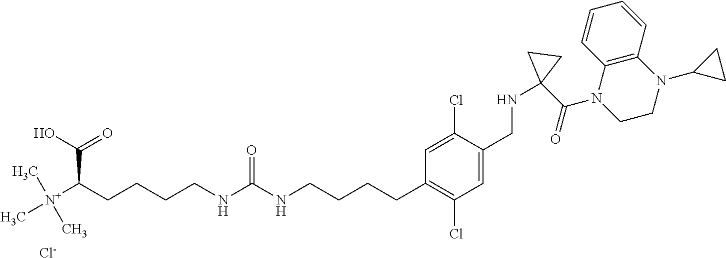 [SMILES: C[N+](C)(C)[C@H](CCCCNC(=O)NCCCCc1cc(Cl)c(CNC2(C(=O)N3CCN(C4CC4)c4ccccc43)CC2)cc1Cl)C(=O)O ; [Cl-]] | [(1R)-1-carboxy-5- {[(4-{2,5-dichloro-4- [({1-[(4-cyclopropyl-1,2,3,4- tetrahydroquinoxalin-1- yl)carbonyl]cyclopropyl} amino)methyl]phenyl} butyl)carbamoyl]amino} pentyl]trimethylazanium |
| 140 | 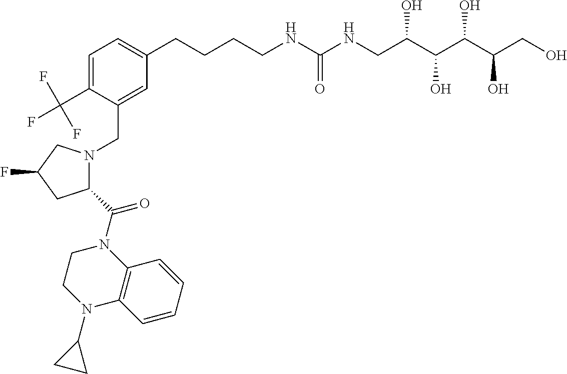 [SMILES: O=C(NCCCCc1ccc(C(F)(F)F)c(CN2C[C@H](F)C[C@H]2C(=O)N2CCN(C3CC3)c3ccccc32)c1)NC[C@H](O)[C@@H](O)[C@H](O)[C@H](O)CO] | 1-[4-(3-{[(2S,4R)-2- [(4-cyclopropyl-1,2,3,4- tetrahydroquinoxalin-1- yl)carbonyl]-4- fluoropyrrolidin-1- yl]methyl}-4- (trifluoromethyl)phenyl) butyl]-3-[(2S,3R,4R,5R)- 2,3,4,5,6- pentahydroxyhexyl]urea |
| 141 | 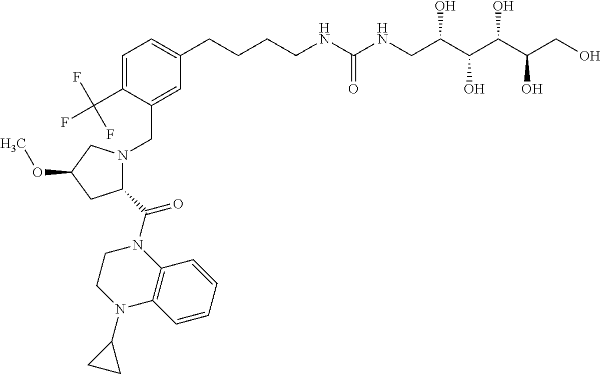 [SMILES: CO[C@@H]1C[C@@H](C(=O)N2CCN(C3CC3)c3ccccc32)N(Cc2cc(CCCCNC(=O)NC[C@H](O)[C@@H](O)[C@H](O)[C@H](O)CO)ccc2C(F)(F)F)C1] | 1-[4-(3-{[(2S,4R)-2- [(4-cyclopropyl-1,2,3,4- tetrahydroquinoxalin-1- yl)carbonyl]-4- methoxypyrrolidin-1- yl]methyl}-4- (trifluoromethyl)phenyl) butyl]-3-[(2S,3R,4R,5R)- 2,3,4,5,6- pentahydroxyhexyl]urea |
| 142 | 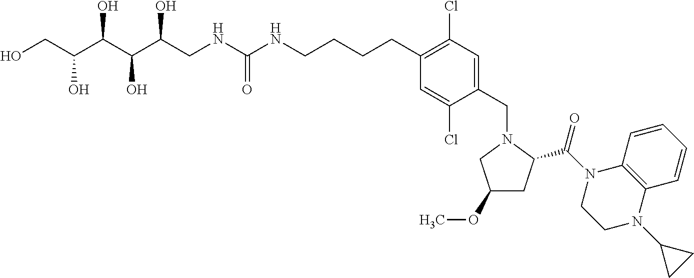 [SMILES: CO[C@@H]1C[C@@H](C(=O)N2CCN(C3CC3)c3ccccc32)N(Cc2cc(Cl)c(CCCCNC(=O)NC[C@H](O)[C@@H](O)[C@H](O)[C@H](O)CO)cc2Cl)C1] | 1-[4-(2,5-dichloro-4- {[(2S,4R)-2-[(4- cyclopropyl-1,2,3,4- tetrahydroquinoxalin-1- yl)carbonyl]-4- methoxypyrrolidin-1- yl]methyl}phenyl)butyl]- 3-[(2S,3R,4R,5R)- 2,3,4,5,6- pentahydroxyhexyl]urea |
| 143 | 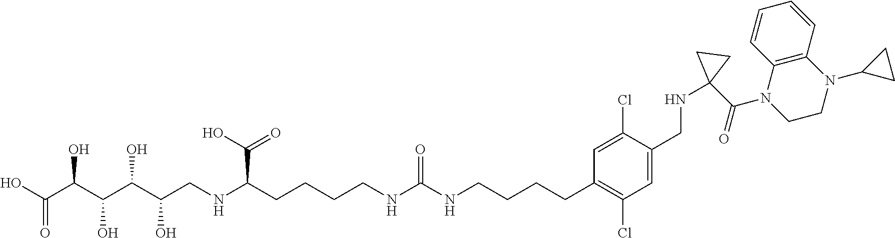 [SMILES: O=C(NCCCCc1cc(Cl)c(CNC2(C(=O)N3CCN(C4CC4)c4ccccc43)CC2)cc1Cl)NCCCC[C@@H](NC[C@H](O)[C@@H](O)[C@H](O)[C@H](O)C(=O)O)C(=O)O] | (2S,3S,4R,5S)-6- {[(1R)-1-carboxy-5- {[(4-{2,5-dichloro-4- [({1-[(4-cyclopropyl-1,2,3,4- tetrahydroquinoxalin-1- yl)carbonyl]cyclopropyl} amino)methyl]phenyl} butyl)carbamoyl]amino} pentyl]amino}-2,3,4,5- tetrahydroxyhexanoic acid |
| 144 | 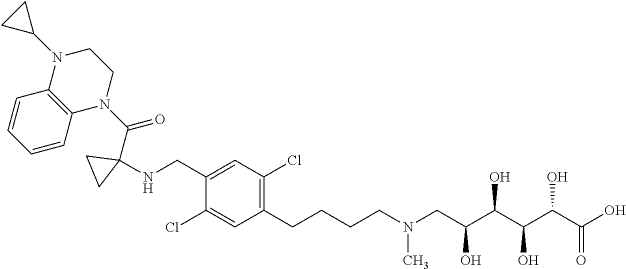 [SMILES: CN(CCCCc1cc(Cl)c(CNC2(C(=O)N3CCN(C4CC4)c4ccccc43)CC2)cc1Cl)C[C@H](O)[C@@H](O)[C@H](O)[C@H](O)C(=O)O] | (2S,3S,4R,5S)-6-[(4- {2,5-dichloro-4-[({1- [(4-cyclopropyl-1,2,3,4- tetrahydroquinoxalin-1- yl)carbonyl]cyclopropyl} amino)methyl]phenyl} butyl)(methyl)amino]- 2,3,4,5-tetrahydroxyhexanoic acid |
| 145 | 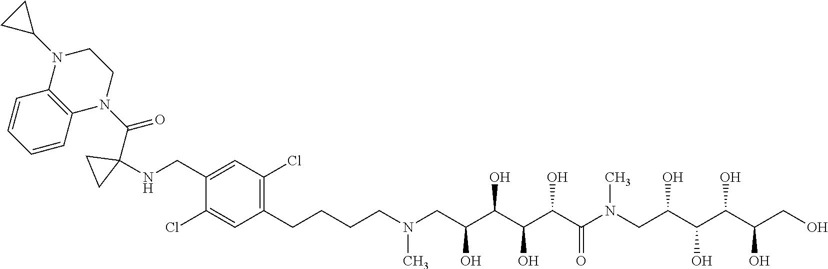 [SMILES: CN(CCCCc1cc(Cl)c(CNC2(C(=O)N3CCN(C4CC4)c4ccccc43)CC2)cc1Cl)C[C@H](O)[C@@H](O)[C@H](O)[C@H](O)C(=O)N(C)C[C@H](O)[C@@H](O)[C@H](O)[C@H](O)CO] | (2S,3S,4R,5S)-6-[(4- {2,5-dichloro-4-[({1- [(4-cyclopropyl-1,2,3,4- tetrahydroquinoxalin-1- yl)carbonyl]cyclopropyl} amino)methyl]phenyl} butyl)(methyl)amino]- 2,3,4,5-tetrahydroxy-N- methyl-N-[(2S,3R,4R,5R)- 2,3,4,5,6-pentahydroxyhexyl]- hexanamide |
| 146 | 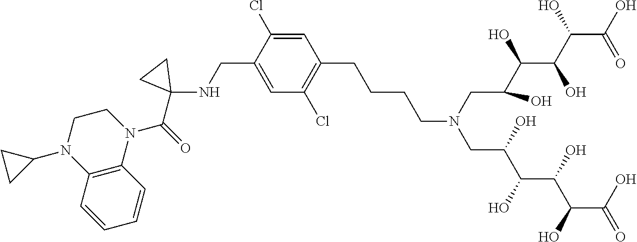 [SMILES: O=C(O)[C@@H](O)[C@@H](O)[C@H](O)[C@@H](O)CN(CCCCc1cc(Cl)c(CNC2(C(=O)N3CCN(C4CC4)c4ccccc43)CC2)cc1Cl)C[C@H](O)[C@@H](O)[C@H](O)[C@H](O)C(=O)O] | (2S,3S,4R,5S)-6- {[(2S,3R,4S,5S)-5- carboxy-2,3,4,5- tetrahydroxypentyl](4- {2,5-dichloro-4-[({1- [(4-cyclopropyl-1,2,3,4- tetrahydroquinoxalin-1- yl)carbonyl]cyclopropyl} amino)methyl]phenyl} butyl)amino}-2,3,4,5- tetrahydroxyhexanoic acid |
| 147 | 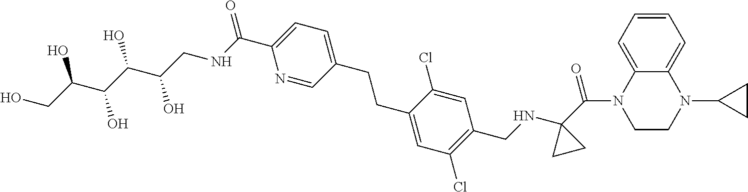 [SMILES: O=C(NC[C@H](O)[C@@H](O)[C@H](O)[C@H](O)CO)c1ccc(CCc2cc(Cl)c(CNC3(C(=O)N4CCN(C5CC5)c5ccccc54)CC3)cc2Cl)cn1] | 5-(2-{2,5-dichloro-4- [({1-[(4-cyclopropyl-1,2,3,4- tetrahydroquinoxalin-1- yl)carbonyl]cyclopropyl} amino)methyl]phenyl} ethyl)-N-[(2S,3R,4R,5R)- 2,3,4,5,6-pentahydroxyhexyl] pyridine-2-carboxamide |
| 148 | 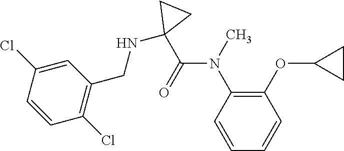 [SMILES: CN(C(=O)C1(NCc2cc(Cl)ccc2Cl)CC1)c1ccccc1OC1CC1] | N-(2-cyclopropoxyphenyl)- 1-{[(2,5- dichlorophenyl)methyl] amino}-N- methylcyclopropane-1- carboxamide |
| 149 | 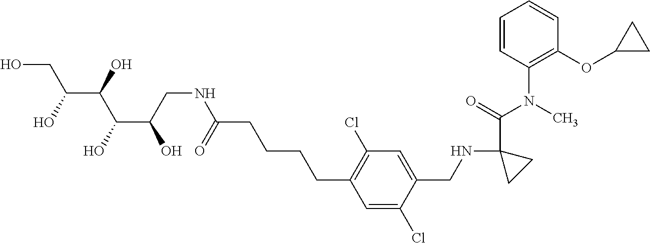 [SMILES: CN(C(=O)C1(NCc2cc(Cl)c(CCCCC(=O)NC[C@@H](O)[C@H](O)[C@H](O)[C@H](O)CO)cc2Cl)CC1)c1ccccc1OC1CC1] | N-(2-cyclopropoxyphenyl)- 1-({[2,5-dichloro-4-(4- {[(2S,3R,4R,5R)- 2,3,4,5,6- pentahydroxyhexyl]carba- moyl}butyl)phenyl] methyl}amino)-N- methylcyclopropane-1- carboxamide |
| 150 | 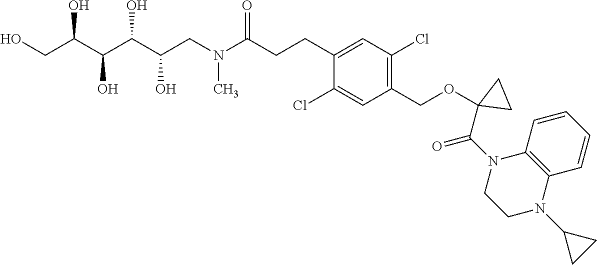 [SMILES: CN(C[C@H](O)[C@@H](O)[C@@H](O)[C@H](O)CO)C(=O)CCc1cc(Cl)c(COC2(C(=O)N3CCN(C4CC4)c4ccccc43)CC2)cc1Cl] | 3-[2,5-dichloro-4-({1- [(4-cyclopropyl-1,2,3,4- tetrahydroquinoxalin-1- yl)carbonyl]cyclopropoxy} methyl)phenyl]-N- methyl-N-[(2S,3R,4S,5R)- 2,3,4,5,6-pentahydroxyhexyl]- propanamide |
| 151 | 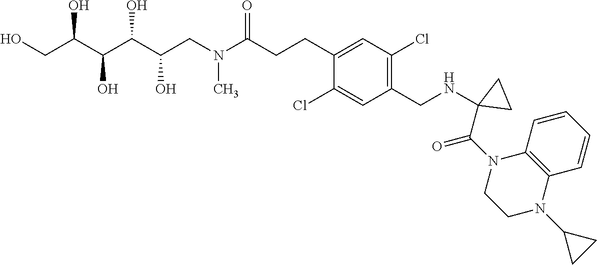 [SMILES: CN(C[C@H](O)[C@@H](O)[C@@H](O)[C@H](O)CO)C(=O)CCc1cc(Cl)c(CNC2(C(=O)N3CCN(C4CC4)c4ccccc43)CC2)cc1Cl] | 3-{2,5-dichloro-4-[({1- [(4-cyclopropyl-1,2,3,4- tetrahydroquinoxalin-1- yl)carbonyl]cyclopropyl} amino)methyl]phenyl}- N-methyl-N-[(2S,3R,4S,5R)- 2,3,4,5,6-pentahydroxyhexyl]- propanamide |
| 152 | 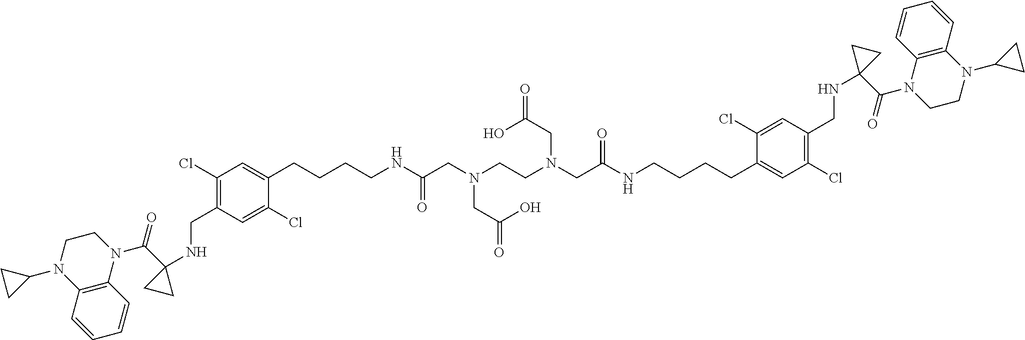 [SMILES: O=C(O)CN(CCN(CC(=O)O)CC(=O)NCCCCc1cc(Cl)c(CNC2(C(=O)N3CCN(C4CC4)c4ccccc43)CC2)cc1Cl)CC(=O)NCCCCc1cc(Cl)c(CNC2(C(=O)N3CCN(C4CC4)c4ccccc43)CC2)cc1Cl] | 2-({2-[(carboxymethyl)({[(4- {2,5-dichloro-4-[({1- [(4-cyclopropyl-1,2,3,4- tetrahydroquinoxalin-1- yl)carbonyl]cyclopropyl} amino)methyl]phenyl} butyl)carbamoyl]meth- yl})amino]ethyl}({[(4- {2,5-dichloro-4-[({1- [(4-cyclopropyl-1,2,3,4- tetrahydroquinoxalin-1- yl)carbonyl]cyclopropyl} amino)methyl]phenyl} butyl)carbamoyl]meth- yl})amino)acetic acid |
| 153 | 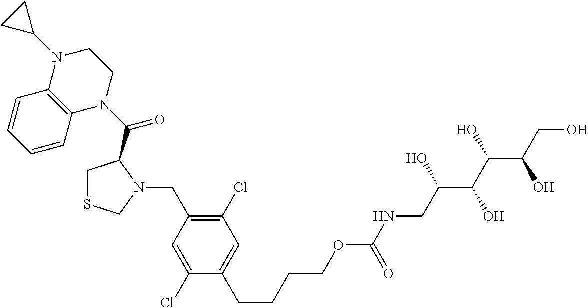 [SMILES: O=C(NC[C@H](O)[C@@H](O)[C@H](O)[C@H](O)CO)OCCCCc1cc(Cl)c(CN2CSC[C@H]2C(=O)N2CCN(C3CC3)c3ccccc32)cc1Cl] | 4-(2,5-dichloro-4-(((R)- 4-(4-cyclopropyl-1,2,3,4- tetrahydroquinoxaline- 1-carbonyl)thiazolidin-3- yl)methyl)phenyl)butyl (2S,3R,4R,5R)-2,3,4,5,6- pentahydroxyhexylcarbamate |
| 154 | 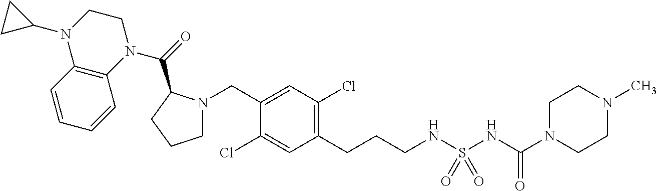 [SMILES: CN1CCN(C(=O)NS(=O)(=O)NCCCc2cc(Cl)c(CN3CCC[C@H]3C(=O)N3CCN(C4CC4)c4ccccc43)cc2Cl)CC1] | (S)-N-(N-(3-(2,5- dichloro-4-((2-(4- cyclopropyl-1,2,3,4- tetrahydroquinoxaline- 1-carbonyl)pyrrolidin-1- yl)methyl)phenyl)propyl) sulfamoyl)-4- methylpiperazine-1- carboxamide |
| 155 | 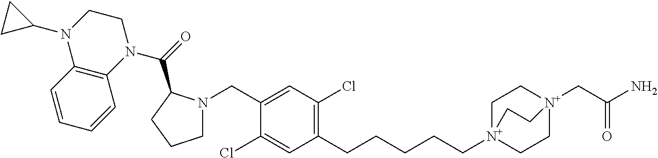 [SMILES: NC(=O)C[N+]12CC[N+](CCCCCc3cc(Cl)c(CN4CCC[C@H]4C(=O)N4CCN(C5CC5)c5ccccc54)cc3Cl)(CC1)CC2] | (S)-1-(2-amino-2- oxoethyl)-4-(5-(2,5- dichloro-4-((2-(4- cyclopropyl-1,2,3,4- tetrahydroquinoxaline- 1-carbonyl)pyrrolidin-1- yl)methyl)phenyl)pentyl)-1,4- diazoniabicyclo[2.2.2]octane |
| 156 | 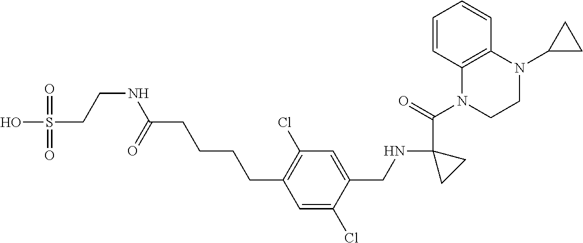 [SMILES: O=C(CCCCc1cc(Cl)c(CNC2(C(=O)N3CCN(C4CC4)c4ccccc43)CC2)cc1Cl)NCCS(=O)(=O)O] | 2-(5-(2,5-dichloro-4- ((1-(4-cyclopropyl-1,2,3,4- tetrahydrquinoxaline-1- carbonyl)cyclopropyl- amino)methyl)phenyl) pentanamido)ethane- sulfonic acid |
| 157 | 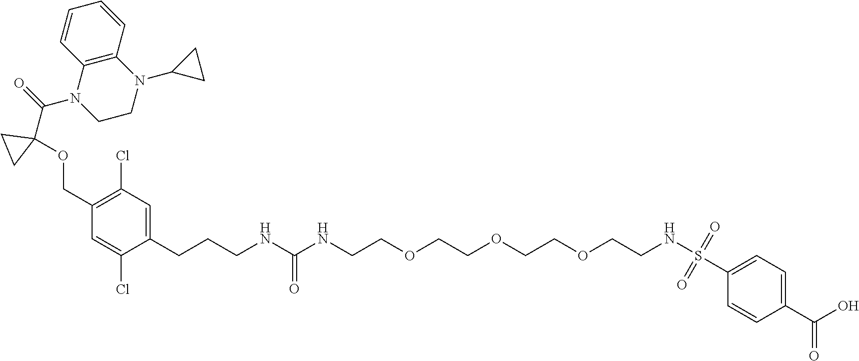 [SMILES: O=C(NCCCc1cc(Cl)c(COC2(C(=O)N3CCN(C4CC4)c4ccccc43)CC2)cc1Cl)NCCOCCOCCOCCNS(=O)(=O)c1ccc(C(=O)O)cc1] | 4-(N-(17-(2,5-dichloro- 4-((1-(4-cyclopropyl-1,2,3,4- tetrahydroquinoxaline-1- carbonyl)cyclopropoxy) methyl)phenyl)-13- oxo-3,6,9-trioxa-12,14- diazaheptadecyl)sulfamoyl) benzoic acid |
| 158 | 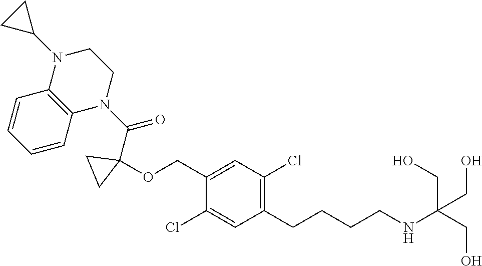 [SMILES: O=C(N1CCN(C2CC2)c2ccccc21)C1(OCc2cc(Cl)c(CCCCNC(CO)(CO)CO)cc2Cl)CC1] | (4-cyclopropyl-3,4- dihydroquinoxalin-1(2H)- yl)(1-(2,5-dichloro-4-(4-(1,3- dihydroxy-2- (hydroxymethyl)propan-2- ylamino)butyl)benzyl- oxy)cyclopropyl) methanone |

### Example 159

### (R)-(4-cyclopropyl-3,4-dihydroquinoxalin-1(2H)-yl)(3-(2,5-dichloro-4-(hydroxymethyl)benzyl)thiazolidin-4-yl)methanone

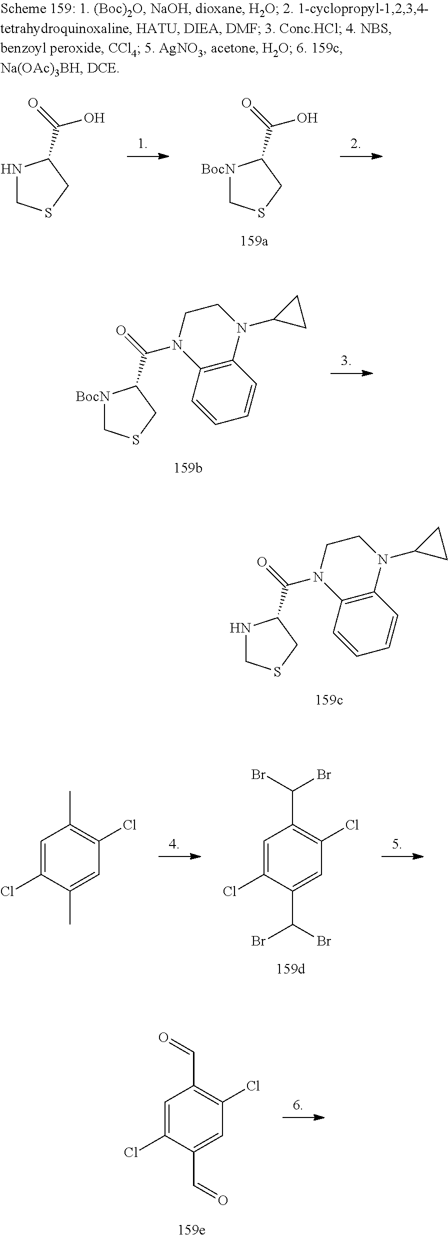

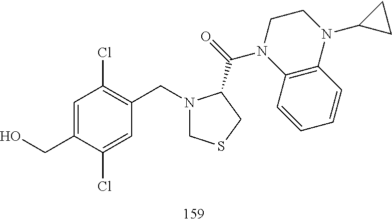 [SMILES: O=C([C@@H]1CSCN1Cc1cc(Cl)c(CO)cc1Cl)N1CCN(C2CC2)c2ccccc21]

Intermediate 159a (4R)-3-[(tert-butoxy)carbonyl]-1,3-thiazolidine-4-carboxylic acid. To a solution of (4R)-1,3-thiazolidine-4-carboxylic acid (9 g, 67.58 mmol, 1.00 equiv) in dioxane (100 mL) was added sodium hydroxide (8.1 g, 202.5 mmol, 3.00 equiv) in water (350 mL) and then (Boc)₂O (22 g, 100.8 mmol, 1.49 equiv). The resulting solution was stirred overnight at room temperature. The pH value of the solution was adjusted to 4 with hydrogen chloride (1 mol/L) and was then extracted with ethyl acetate (3×250 mL). The combined organic layers were washed with brine (2×500 mL), dried over anhydrous sodium sulfate and concentrated under vacuum to afford 15 g (95%) of 159a as a white solid with was used without further purification.

Intermediate 159b (tert-butyl(4R)-4-[(4-cyclopropyl-1,2,3,4-tetrahydroquinoxalin-1-yl)carbonyl]-1,3-thiazolidine-3-carboxylate. A solution of (4R)-3-[(tert-butoxy)carbonyl]-1,3-thiazolidine-4-carboxylic acid (8.0 g, 34.29 mmol, 1.00 equiv), 1-cyclopropyl-1,2,3,4-tetrahydroquinoxaline (6 g, 34.43 mmol, 1.00 equiv), HATU (17 g, 44.71 mmol, 1.30 equiv), and DIEA (6.7 g, 51.84 mmol, 1.51 equiv) was stirred in DMF (80 mL) overnight. The resulting solution was diluted with H₂O (500 mL), extracted with ethyl acetate (2×250 mL) and the combined organic layers were washed with brine (2×500 mL), dried over anhydrous sodium sulfate, and concentrated under vacuum. The residue was purified by silica gel column chromatography with an eluent gradient of petroleum ether/ethyl acetate (20:1 to 10:1) to furnish 159b (12 g, 90%) as a yellow oil.

Intermediate 159c 1-cyclopropyl-4-[[(4R)-1,3-thiazolidin-4-yl]carbonyl]-1,2,3,4-tetrahydroquinoxaline. To a solution of tert-butyl(4R)-4-[(4-cyclopropyl-1,2,3,4-tetrahydroquinoxalin-1-yl)carbonyl]-1,3-thiazolidine-3-carboxylate (10 g, 25.67 mmol, 1.00 equiv) in 1,4-dioxane (150 mL) was added concentrated HCl (50 mL). The resulting solution was stirred for 1 h at room temperature, then the pH value of the solution was adjusted to 6-7 with aqueous sodium hydroxide and the resulting solution was extracted with ethyl acetate (2×300 mL). The organic layers were combined, washed with brine (3×500 mL), dried over anhydrous sodium sulfate and concentrated under vacuum. The residue was purified by silica gel column chromatography with an eluent gradient of ethyl acetate/petroleum ether (1:10 to 1:4) to furnish 159c (4.98 g, 67%) as light yellow oil. MS (ES, m/z): 290 [M+H]⁺. ¹H-NMR (400 MHz, CDCl₃): 7.28-7.11 (m, 3H), 6.74-6.70 (m, 1H), 4.45-4.43 (d, J=9.6 Hz, 1H), 4.14-4.00 (m, 3H), 3.80-3.77 (m, 1H), 3.44-3.41 (t, J=5, 6, 6 Hz, 2H), 2.97-2.93 (t, J=9.6, 6.8 Hz, 1H), 2.74-2.69 (t, J=9.6, 9.2 Hz, 2H), 2.48-2.44 (m, 1H), 0.88-0.84 (m, 2H), 0.69-0.60 (m, 2H).

Intermediate 159d: 1,4-dichloro-2,5-bis(dibromomethyl)benzene. To a solution of 1,4-dichloro-2,5-dimethylbenzene (5 g, 28.56 mmol, 1.00 equiv) in CCl₄ (50 mL) was added NBS (25.4 g, 142.71 mmol, 5.00 equiv) and benzoyl peroxide (490 mg, 2.02 mmol, 0.07 equiv) and the resulting solution was stirred overnight at 80° C. in an oil bath. The solids were filtered out, the filter cake was washed with 4×100 mL of ethyl acetate, and the organic layers were combined, washed with 2×100 mL of water, 1×150 mL of saturated Na₂S₂O₃ and 1×150 mL of brine. The mixture was dried over anhydrous sodium sulfate and concentrated under vacuum to give 15 g of intermediate 159d as a light yellow solid, which was used without further purification.

Intermediate 159e: 2,5-dichlorobenzene-1,4-dicarbaldehyde. To a solution of intermediate 159d (15 g, 30.57 mmol, 1.00 equiv) in acetone (100 mL) was added a solution of AgNO₃ (21.8 g, 128.31 mmol, 4.20 equiv) in water (30 mL) dropwise with stirring at 65° C. The resulting solution was stirred for 2 h at 65° C. in an oil bath. The resulting solution was diluted with 500 mL of ethyl acetate. The resulting mixture was washed with 1×100 mL of water, 1×120 mL of hydrogen chloride (1N), 1×100 mL of NaHCO₃ (sat.) and 1×100 mL of brine. The mixture was dried over anhydrous sodium sulfate and concentrated under vacuum. The residue was applied onto a silica gel column with ethyl acetate/petroleum ether (1:20˜EA) to afford 2.5 g (40%) of intermediate 159e as a light yellow solid.

Example 159: (R)-(4-cyclopropyl-3,4-dihydroquinoxalin-1(2H)-yl)(3-(2,5-dichloro-4-(hydroxymethyl)benzyl)thiazolidin-4-yl)methanone: To a solution of intermediate 159e (2.4 g, 11.82 mmol, 1.20 equiv) in 1,2-dichloroethane (60.0 mL) was added 159c (3.0 g, 10.37 mmol, 1.00 equiv) and the mixture was stirred for 1 h. To this was added NaBH(OAc)₃ (8.8 g, 41.53 mmol, 4.00 equiv) in several batches and the resulting solution was stirred overnight. The reaction was then quenched by the addition of 100 mL of water and extracted with 3×100 mL of dichloromethane. The combined organic layers were washed with 1×100 mL of brine, dried over anhydrous sodium sulfate, and concentrated under vacuum. The residue was applied onto a silica gel column with ethyl acetate/petroleum ether (1:20-1:8) to afford 2.1 g (42%) of Example 159 as a white solid.

### Example 160

### (R)-(3-(4-(5-aminopentyl)-2,5-dichlorobenzyl)thiazolidin-4-yl)(4-cyclopropyl-3,4-dihydroquinoxalin-1(2H)-yl)methanone

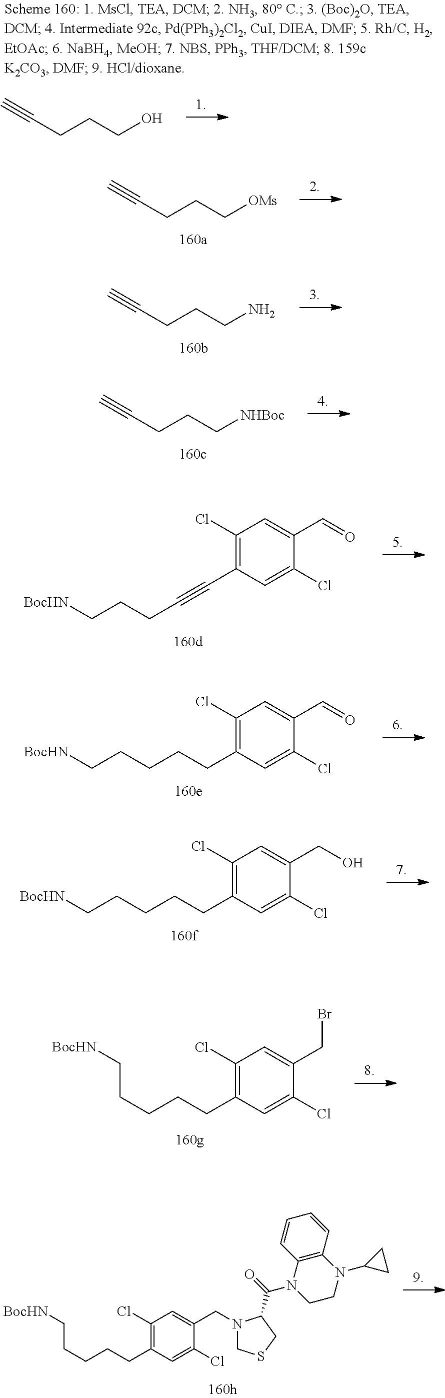

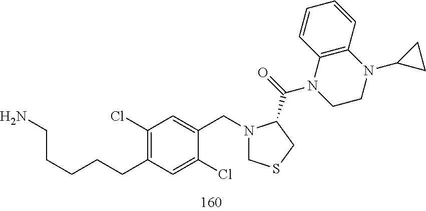 [SMILES: NCCCCCc1cc(Cl)c(CN2CSC[C@H]2C(=O)N2CCN(C3CC3)c3ccccc32)cc1Cl]

Intermediate 160a: pent-4-ynyl methanesulfonate. To pent-4-yn-1-ol (10 g, 118.88 mmol, 1.00 equiv) in DCM (150 mL) at 0° C. was added TEA (18.04 g, 178.28 mmol, 1.50 equiv) followed by the drop-wise addition of a solution of methanesulfonyl chloride (16.36 g, 142.82 mmol, 1.20 equiv) in DCM (50 mL) and the resulting solution was stirred for 1.5 h. The reaction was quenched by the addition of water (100 mL) and then extracted with 2×100 mL of dichloromethane. The organic layers were combined, washed with 1×200 mL of sodium bicarbonate (sat.), 1×250 mL of brine, dried over anhydrous sodium sulfate and then concentrated to afford 18.05 g (94%) of intermediate 160a as a brown oil.

Intermediate 160b: pent-4-yn-1-amine To intermediate 160a (11.4 g, 70.28 mmol, 1.00 equiv) in a 250 mL high-pressure sealable tube was added liquid ammonia (60 mL), the tube was sealed, and the mixture was stirred overnight at 80° C. The reaction mixture was then cooled to 0° C. and the tube opened, the contents diluted with 150 mL of ether, the mixture was filtered and then the filtrate was concentrated under vacuum to afford 4.91 g (84%) of intermediate 160b as brown oil.

Intermediate 160c: tert-butyl pent-4-ynylcarbamate. To intermediate 160b (4.91 g, 59.06 mmol, 1.00 equiv) in DCM (40 mL) at 0° C. was added TEA (8.95 g, 88.45 mmol, 1.50 equiv) followed by the drop-wise addition of a solution of di-tert-butyl dicarbonate (12.88 g, 59.02 mmol, 1.00 equiv) in DCM (20 mL). The resulting solution was allowed to warm to RT and then stirred overnight at room temperature. The mixture was concentrated under vacuum and then purified via silica gel chromatography (ethyl acetate/petroleum ether 1:50-1:40) to afford 4.59 g (43%) of intermediate 160c as a light yellow oil.

Intermediate 160d: tert-butyl 5-(2,5-dichloro-4-formylphenyl)pent-4-ynylcarbamate. To intermediate 92c (7.71 g, 23.86 mmol, 1.00 equiv) in DMF (100 mL) was added intermediate 160c (4.59 g, 25.05 mmol, 1.05 equiv), Pd(PPh₃)₂Cl₂ (1.67 g, 2.38 mmol, 0.10 equiv), CuI (450 mg, 2.36 mmol, 0.10 equiv) and DIEA (6.61 g, 51.15 mmol, 2.00 equiv) and the resulting solution was stirred overnight. The mixture was diluted with 500 mL of ethyl acetate, washed with 3×200 mL of brine and the organic layer was dried over sodium sulfate and then concentrated under vacuum. The residue was purified via silica gel chromatography (petroleum ether/ethyl acetate. 50:1˜10:1) to afford 3.7 g (44%) of intermediate 160d as a brown syrup.

Intermediate 160e: tert-butyl 5-(2,5-dichloro-4-formylphenyl)pentylcarbamate. To intermediate 160d (3.21 g, 9.01 mmol, 1.00 equiv) in ethyl acetate (90 mL) was added Rh/C (3.60 g) and the suspension stirred under a hydrogen atmosphere overnight. The solids were filtered out and the filtrate was concentrated under vacuum to afford 3.1 g (95%) of intermediate 160e as a brown oil.

Intermediate 160f: tert-butyl 5-(2,5-dichloro-4-(hydroxymethyl)phenyl)pentylcarbamate. To intermediate 160e (3.1 g, 8.60 mmol, 1.00 equiv) in methanol (100 mL) at 0° C. was added portion-wise NaBH₄ (810 mg, 21.41 mmol, 2.49 equiv) over 30 min. The resulting mixture was stirred for 1 h at 0° C., then quenched by the addition of 50 mL of water. The mixture was concentrated under vacuum to remove the organic solvents, then extracted with 3×100 mL of dichloromethane. The organic layers were combined, washed with 3×100 mL of brine, dried over anhydrous sodium sulfate and then concentrated to afford 2.70 g (87%) of intermediate 160f as light yellow oil.

Intermediate 160g: tert-butyl 5-(4-(bromomethyl)-2,5-dichlorophenyl)pentylcarbamate. To intermediate 160f (250 mg, 0.69 mmol, 1.00 equiv) in DCM/THF (2/2 mL) at 0° C. was added NBS (235 mg, 1.32 mmol, 1.90 equiv) followed by the batch-wise addition of triphenylphosphine (373 mg, 1.42 mmol, 1.50 equiv). The reaction was allowed to warm to RT and then stirred for 1 h. The resulting mixture was concentrated under vacuum and the residue purified via silica gel chromatography (ethyl acetate/petroleum ether, 1:50) to afford 173 mg (59%) of intermediate 160g as light yellow oil.

Intermediate 160h: (R)-tert-butyl 5-(2,5-dichloro-4-((4-(4-cyclopropyl-1,2,3,4-tetrahydroquinoxaline-1-carbonyl)thiazolidin-3-yl)methyl)phenyl)pentylcarbamate. To intermediate 160g (3 g, 7.06 mmol, 1.00 equiv) in DMF (10 mL) was added intermediate 159c (2 g, 6.91 mmol, 1.00 equiv) and potassium carbonate (2 g, 14.47 mmol, 2.00 equiv) and the reaction was stirred overnight. The mixture was diluted with 20 mL of water, extracted with 3×30 mL of ethyl acetate, the organic layers combined, washed with 1×50 mL of brine and then dried. The solution was concentrated under vacuum and the residue was purified via silica gel chromatography (ethyl acetate/petroleum ether, 1:20) to afford 1.7 g (38%) of intermediate 160h as a brown solid.

Example 160: (R)-(3-(4-(5-aminopentyl)-2,5-dichlorobenzyl)thiazolidin-4-yl)(4-cyclopropyl-3,4-dihydroquinoxalin-1(2H)-yl)methanone. To intermediate 160h (1.7 g, 2.68 mmol, 1.00 equiv) was added 1 M HCl in dioxne (5 mL) and the resulting solution was stirred for 1 h. The resulting mixture was concentrated under vacuum, diluted with 30 mL of ethyl acetate, and washed with 3×10 mL of aqueous sodium carbonate. The organic layer was dried over sodium sulfate and concentrated under vacuum to afford 1.4 g (98%) of example 160 as a yellow solid. LCMS (ES, m/z): 533 [M+1]⁺. ¹H-NMR (300 MHz, CDCl₃, ppm): 7.49 (s, 1H), 7.21 (s, 1H), 4.74 (s, 2H), 4.46˜4.40 (m, 1H), 3.13 (s, 2H), 2.72˜2.67 (m, 2H), 1.87 (s, 1H), 1.68˜1.53 (m, 4H), 1.51 (s, 9H), 1.4˜61.36 (m, 2H).

### Example 161

### (R)-2-((2,5-dichloro-4-((4-(4-cyclopropyl-1,2,3,4-tetrahydroquinoxaline-1-carbonyl)thiazolidin-3-yl)methyl)benzyl)thio)-1-methyl-1H-imidazole-5-carboxylic acid

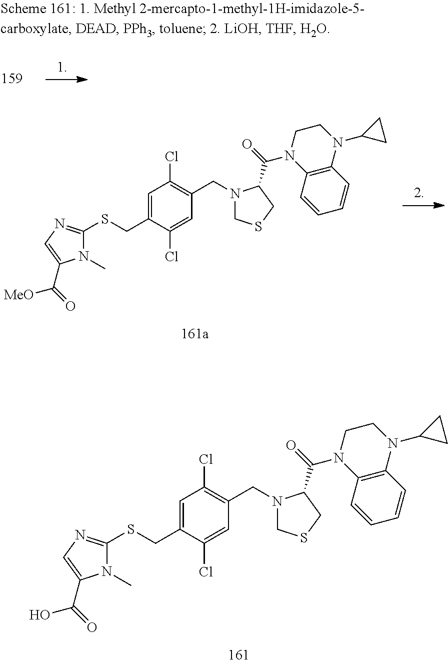 [SMILES: COC(=O)c1cnc(SCc2cc(Cl)c(CN3CSC[C@H]3C(=O)N3CCN(C4CC4)c4ccccc43)cc2Cl)n1C ; Cn1c(C(=O)O)cnc1SCc1cc(Cl)c(CN2CSC[C@H]2C(=O)N2CCN(C3CC3)c3ccccc32)cc1Cl]

Intermediate 161a: (R)-methyl 2-((2,5-dichloro-4-((4-(4-cyclopropyl-1,2,3,4-tetrahydroquinoxaline-1-carbonyl)thiazolidin-3-yl)methyl)benzyl)thio)-1-methyl-1H-imidazole-5-carboxylate. To a mixture of example 159 (60 mg, 0.125 mmol, 1 equiv), methyl 2-mercapto-1-methyl-1H-imidazole-5-carboxylate (30.2 mg, 0.176 mmol, 1.4 equiv) and PPh₃ (46.2 mg, 0.176 mmol, 1.4 equiv) in toluene (0.35 mL) at 0° C. was added dropwise diethyl azodicarboxylate (40% wt in toluene, 80 μL, 0.176 mmol 1.4 equiv). The mixture was stirred at rt for 3 h, concentrated, and then purified by column to give 79 mg (100%) of intermediate 161a as a white solid.

Example 161: (R)-2-((2,5-dichloro-4-((4-(4-cyclopropyl-1,2,3,4-tetrahydroquinoxaline-1-carbonyl)thiazolidin-3-yl)methyl)benzyl)thio)-1-methyl-1H-imidazole-5-carboxylic acid. To a mixture of intermediate 161a (79 mg, 0.125 mmol, 1 equiv) in THF (0.4 mL) and water (0.2 mL) was added LiOH.H₂O (26.2 mg, 0.625 mmol, 5 equiv) and the reaction was stirred overnight. The mixture was concentrated, diluted with H₂O (0.3 mL), acidified by 1M HCl to pH=3, and then extracted with EtOAc. The organic layer was washed with brine (1×), dried and concentrated to give 51 mg (66%) of example 161 as a white solid. LCMS (ES, m/z): 618.10 [M+H]⁺.

### Example 162

### (R)-(4-cyclopropyl-3,4-dihydroquinoxalin-1(2H)-yl)(3-(2,5-dichloro-4-hydroxybenzyl)thiazolidin-4-yl)methanone

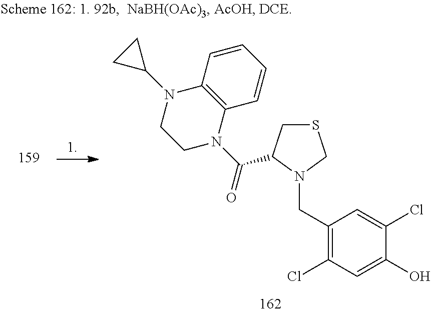

Example 162: (R)-(4-cyclopropyl-3,4-dihydroquinoxalin-1(2H)-yl)(3-(2,5-dichloro-4-hydroxybenzyl)thiazolidin-4-yl)methanone. To a solution of 159c (200 mg, 0.691 mmol), 92b (132 mg, 0.691 mmol) and AcOH (40 μL, 0.69 mmol) in DCE (3 mL) was added NaBH(OAc)₃ (234 mg, 1.11 mmol) and the resulting mixture stirred for 16 h. The excess NaBH(OAc)₃ was quenched with 1M aqueous HCl, and the mixture then extracted with DCM. The organic layer was dried over Na₂SO₄, filtered, and then the solvent removed under reduced pressure. The resulting residue was purified by flash column chromatography, using 10 to 50% EtOAc in hexanes as eluent to give Example 162 as a white powder (140 mg, 44%). MS (ES, m/z): 464.16 [M+H]′.

### Example 163

### (R)-5-(2,5-dichloro-4-((4-(4-cyclopropyl-1,2,3,4-tetrahydroquinoxaline-1-carbonyl)thiazolidin-3-yl)methyl)phenyl)pentanoic acid

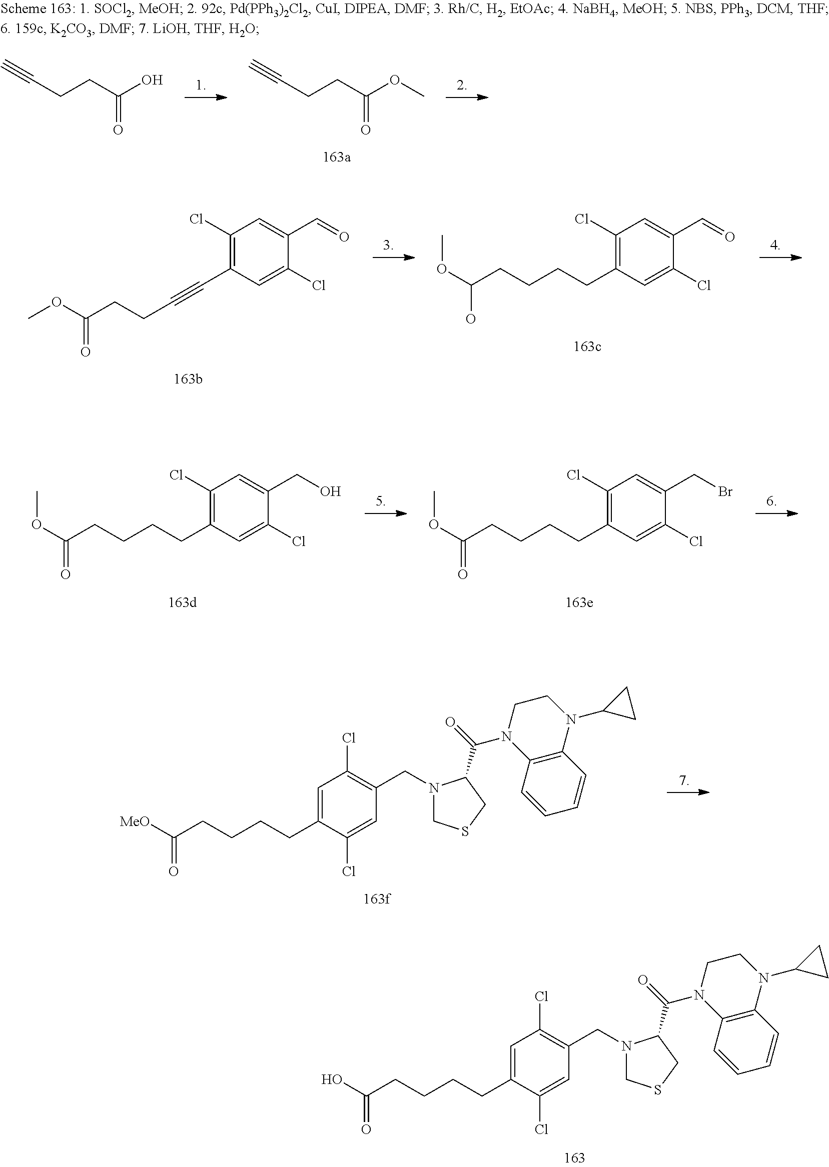

Intermediate 163a: methyl pent-4-ynoate. To a mixture of pent-4-ynoic acid (5 g, 50.97 mmol, 1.00 equiv) in methanol (250 mL) at 0° C. was added thionyl chloride (4.45 mL) dropwise and the resulting solution was stirred overnight. The mixture was diluted with 800 mL of dichloromethane, washed with 2×500 mL of water. dried over anhydrous sodium sulfate and then concentrated to give 5.9 g (crude) of intermediate 163a as light yellow oil

Intermediate 163b: methyl 5-(2,5-dichloro-4-formylphenyl)pent-4-ynoate. To a mixture of 92c (6.0 g, 18.57 mmol, 1.00 equiv), intermediate 163a (2.50 g, 22.30 mmol, 1.20 equiv) and DIEA (4.79 g, 37.06 mmol, 2.00 equiv) in DMF (45 mL) was added Pd(PPh₃)₂Cl₂ (1.30 g, 1.85 mmol, 0.10 equiv) and Cut (354 mg, 1.86 mmol, 0.10 equiv) and the resulting solution was stirred overnight. The mixture was diluted with 300 mL of ethyl acetate, washed with 2×200 mL of water and 2×200 mL of brine, the organic layer dried over anhydrous sodium sulfate and then concentrated under vacuum. The residue was applied onto a silica gel column with petroleum ether/ethyl acetate (40:1) to afford 3.05 g (58%) of intermediate 163b as a light yellow solid

Intermediate 163c: methyl 5-(2,5-dichloro-4-formylphenyl)pentanoate: To a mixture of intermediate 163b (3.05 g, 10.70 mmol, 1.00 equiv) in ethyl acetate (100 mL) was added Rh/C (3.23 g) and the suspension was stirred under a H₂ atmosphere overnight. The solids were filtered out and the filtrate concentrated to provide 2.52 g (81%) of intermediate 163c as brown oil.

Intermediate 163d: methyl 5-[2,5-dichloro-4-(hydroxymethyl)phenyl]pentanoate. To a solution of intermediate 163c (2.52 g, 8.72 mmol, 1.00 equiv) in methanol (40 mL) at 0° C. was added NaBH₄ (660 mg, 17.45 mmol, 2.00 equiv) in several batches over 1 h. The reaction was stirred for 1 h at 0-5° C. and then quenched by the addition of 50 mL of water/ice. The mixture was concentrated under vacuum to remove the organic solvents and then extracted with 3×50 mL of DCM. The organic layers were combined, washed with 1×100 mL of brine, dried over anhydrous sodium sulfate and then concentrated to afford 2.42 g (95%) of intermediate 163d as an off-white solid.

Intermediate 163e: methyl 5-(4-(bromomethyl)-2,5-dichlorophenyl)pentanoate. To a mixture of intermediate 163d (200 mg, 0.686 mmol, 1 equiv) in DCM (1.3 mL) and THF (1.3 mL) at 0° C. was added NBS (269 mg, 1.51 mmol, 2.2 equiv) and PPh₃ (234 mg, 0.892 mmol, 1.3 equiv) and the mixture was stirred for 1 h. The reaction was quenched with brine, extracted with EtOAc, the organic layer was dried, concentrated, and purified by column to give 227 mg (93%) of intermediate 163e as clear oil.

Intermediate 163f: (R)-methyl 5-(2,5-dichloro-4-((4-(4-cyclopropyl-1,2,3,4-tetrahydroquinoxaline-1-carbonyl)thiazolidin-3-yl)methyl)phenyl)pentanoate. To a mixture of 159c (20.3 mg, 0.07 mmol, 1 equiv) in DMF (0.3 mL) was added intermediate 163e (27.3 mg, 0.077 mmol, 1.1 equiv) and K₂CO₃ (19.4 mg, 0.14 mmol, 2 equiv) and the mixture stirred for 3 h and then heated to 60° C. and stirred overnight. The mixture was diluted with EtOAc, washed with H₂O (2 x) and brine (1 x), the organic layer was dried, concentrated, and purified by column to give 12.6 mg (32%) of intermediate 163f as clear syrup.

Example 163: (R)-5-(2,5-dichloro-4-((4-(4-cyclopropyl-1,2,3,4-tetrahydroquinoxaline-1-carbonyl)thiazolidin-3-yl)methyl)phenyl)pentanoic acid. To a mixture of intermediate 163f (10.8 mg, 0.0193 mmol, 1 equiv) in THF (0.12 mL) and water (0.06 mL) was added LiOH.H₂O (1.6 mg, 0.0385 mmol, 2 equiv) and the reaction was stirred for 6 h. The mixture was acidified with 1M HCl (42 mL), concentrated and then lyophilized to give 11.4 mg of Example 163 as a white solid. LCMS (ES, m/z): 548.09 [M+h]⁺.

### Example 164

### (4-cyclopropyl-3,4-dihydroquinoxalin-1(2H)-yl)(1-(5-iodo-2-(trifluoromethyl)benzylamino)cyclopropyl)methanone

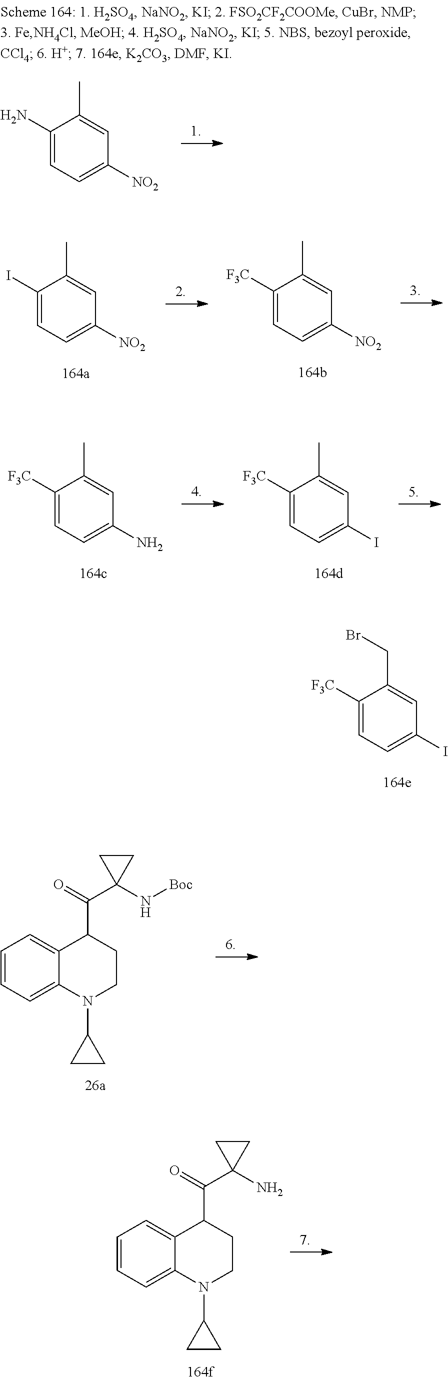

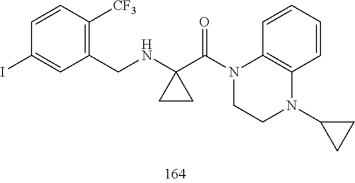 [SMILES: O=C(N1CCN(C2CC2)c2ccccc21)C1(NCc2cc(I)ccc2C(F)(F)F)CC1]

Intermediate 164a: 1-iodo-2-methyl-4-nitrobenzene. To 2-methyl-4-nitroaniline (20.0 g, 131.45 mmol, 1.00 equiv) in H₂O/acetone (80/50 mL) at 0-5° C. was added conc. H₂SO₄ (27.1 g, 276.53 mmol, 2.10 equiv) followed by the drop-wise addition of a solution of NaNO₂ (10.0 g, 144.93 mmol, 1.10 equiv) in water (20 mL) and the resulting solution was stirred for 1 h. To this was added drop-wise a solution of KI (30.6 g, 184.34 mmol, 1.40 equiv) in water (20 mL) and the reaction allowed to warm to RT and then stirred for an additional 2 h. The mixture was diluted with 500 mL of ethyl acetate, washed with 2×200 mL of water, 3×200 mL of aqueous Na₂SO₃, dried over anhydrous sodium sulfate and concentrated. The residue was purified via silica gel chromatography (ethyl acetate/petroleum ether, 1:1000) to afford 21.7 g (63%) of intermediate 164a as a white solid.

Intermediate 164b: 2-methyl-4-nitro-1-(trifluoromethyl)benzene. To intermediate 164a (21.9 g, 83.26 mmol, 1.00 equiv) in NMP (150 mL) was added methyl 2,2-difluoro-2-(fluorosulfonyl)acetate (23.73 g, 123.52 mmol, 1.50 equiv) and CuBr (1.45 g, 10.11 mmol, 0.12 equiv) and the mixture was stirred at 120° C. overnight. The mixture was diluted with 500 mL of ethyl acetate, washed with 3×200 mL of brine, dried over anhydrous sodium sulfate and concentrated. The residue was purified via silica gel chromatography (ethyl acetate/petroleum ether, 1:100) to afford 15.0 g (88%) of intermediate 164b as a yellow oil.

Intermediate 164c: 3-methyl-4-(trifluoromethyl)aniline. To intermediate 164b (15.0 g, 73.12 mmol, 1.00 equiv) in methanol/H₂O (100/25 mL) was added elemental Fe (15.0 g, 267.86 mmol, 3.66 equiv) and NH₄Cl (15.0 g, 280.43 mmol, 3.83 equiv) and the reaction was stirred at 60° C. for 3 h. The mixture was filtered and the filtrate concentrated, diluted with 200 mL of ethyl acetate, washed with 2×100 mL of brine, dried over sodium sulfate and then concentrated to afford 8.0 g (62%) of intermediate 164c as a yellow oil.

Intermediate 164d: 4-iodo-2-methyl-1-(trifluoromethyl)benzene. To intermediate 164c (6.0 g, 34.26 mmol, 1.00 equiv) in water (50 mL) at 0° C. was added sulfuric acid (7.06 g, 71.98 mmol, 2.10 equiv) followed by the drop-wise addition of a solution of NaNO₂ (2.60 g, 37.68 mmol, 1.10 equiv) in water (40 mL) and the mixture was stirred for 1 h. To this was added drop-wise a solution of KI (7.97 g, 48.01 mmol, 1.40 equiv) in water (40 mL) and the reaction was allowed to warm to RT and stirred for 1 h. The mixture was diluted with 200 mL of ethyl acetate, washed with 2×200 mL of Brine, 1×200 mL of aqueous Na₂SO₃, dried over anhydrous sodium sulfate and concentrated. The residue was purified via silica gel chromatography (petroleum ether/ethyl acetate, 100:1) to afford 8.2 g (85%) of intermediate 164d as a yellow oil.

Intermediate 164e: 2-(bromomethyl)-4-iodo-1-(trifluoromethyl)benzene. To intermediate 164d (3.5 g, 12.24 mmol, 1.00 equiv) in CCl₄ (40 mL) at 60° C. was added benzoyl peroxide (1.7 g, 7.02 mmol, 0.57 equiv) followed by the batch-wise addition of NBS (2.37 g, 13.32 mmol, 1.09 equiv) and the reaction stirred at reflux overnight. The solids were filtered out and the filtrate concentrated to afford 1.6 g (36%) of intermediate 164e as a red oil.

Intermediate 164f: (1-aminocyclopropyl)(4-cyclopropyl-3,4-dihydroquinoxalin-1(2H)-yl)methanone. Intermediate 164f was prepared from intermediate 26a using the procedures described in Example 59.

Example 164: (4-cyclopropyl-3,4-dihydroquinoxalin-1(2H)-yl)(1-(5-iodo-2-(trifluoromethyl)benzylamino)cyclopropyl)methanone. To intermediate 164e (364 mg, 1.00 mmol, 1.00 equiv) in DMF (5 mL) was added intermediate 164f (257 mg, 1.00 mmol, 1.00 equiv), potassium carbonate (208 mg, 1.50 mmol, 1.50 equiv) and KI (166 mg, 1.00 mmol, 1.00 equiv) and the reaction was stirred overnight. The mixture was diluted with 20 mL of ethyl acetate, washed with 2×20 mL of brine, dried over anhydrous sodium sulfate, concentrated and then purified via silica gel chromatography (petroleum ether/ethyl acetate, 5:1) to afford Example 164 of a purity suitable for use in the next step. A 300 mg aliquot was further purified via reverse phase (C18) Prep-HPLC to afford 150 mg (28%) of the title compound as an off-white solid. LCMS (ES, m/z): 542 [M+1]⁺. ¹H NMR (400 MHz, CD₃OD, ppm): 8.33-7.78 (m, 1H), 7.70-7.22 (m, 5H), 7.07-6.78 (m, 1H), 4.11-3.82 (m, 4H), 3.45-3.09 (m, 2H), 2.44 (s, 1H), 1.41 (s, 2H), 1.04 (s, 2H), 0.83 (d, J=6.9 Hz, 2H), 048 (s, 2H).

### Example 165

### (4-Cyclopropyl-3,4-dihydroquinoxalin-1(2H)-yl)(1-((2,5-dichloro-4-hydroxybenzyl)amino)cyclopropyl)methanone

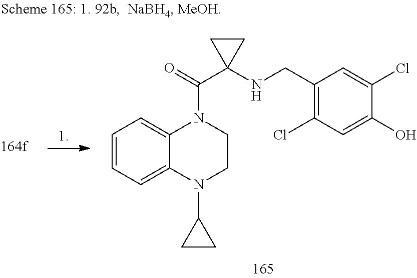 [SMILES: O=C(N1CCN(C2CC2)c2ccccc21)C1(NCc2cc(Cl)c(O)cc2Cl)CC1]

Example 165: (4-cyclopropyl-3,4-dihydroquinoxalin-1(2H)-yl)(1-((2,5-dichloro-4-hydroxybenzyl)amino)cyclopropyl)methanone bis-TFA salt. To a mixture of intermediate 164f (50.6 mg, 0.197 mmol, 1.0 equiv) in methanol (0.8 mL) was added 92b (37.6 mg, 0.197 mmol, 1.0 equiv). The mixture was stirred at room temperature for 1.5 h and cooled to 0° C. To the mixture was added acetic acid (11.3 μL, 0.197 mmol, 1.0 equiv), followed by addition of sodium borohydride (11.9 mg, 0.315 mmol, 1.6 equiv). The mixture was stirred at 0° C. for 20 minutes and purified by preparative. HPLC to give the title compound (74.7 mg, 57%) bis-TFA salt as a pale yellow solid. MS (ES, m/z): 432.03 [M+H]⁺, ¹H NMR (400 MHz, CD₃OD) δ 7.27 (td, J=8.4, 1.4 Hz, 2H), 7.20 (s, 1H), 7.19-7.13 (m, 1H), 6.96 (s, 1H), 6.75 (td, J=7.6, 1.3 Hz, 1H), 4.04 (s, 2H), 3.89 (t, J=5.8 Hz, 2H), 3.44 (t, J=5.8 Hz, 2H), 2.50-2.36 (m, 1H), 1.35 (dd, J=7.8, 5.6 Hz, 2H), 1.24 (dd, J=7.6, 5.7 Hz, 2H), 0.91-0.79 (m, 2H), 0.61-0.53 (m, 2H).

### Example 166

### 1-(4-cyclopropyl-1,2,3,4-tetrahydroquinoxaline-1-carbonyl)cyclopropyl 2,5-dichlorobenzoate

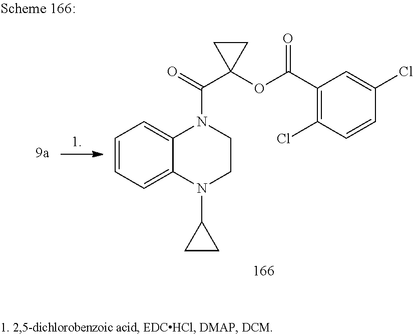 [SMILES: O=C(OC1(C(=O)N2CCN(C3CC3)c3ccccc32)CC1)c1cc(Cl)ccc1Cl]

Example 166: 1-(4-cyclopropyl-1,2,3,4-tetrahydroquinoxaline-1-carbonyl)cyclopropyl 2,5-dichlorobenzoate TFA salt. To a mixture of intermediate 9a (28.3 mg, 0.11 mmol, 1.0 equiv) in DCM (0.4 mL) were added 2,5-dichlorobenzoic acid (42 mg, 0.22 mmol, 2.0 equiv) and DMAP (27 mg, 0.22 mmol, 2.0 equiv). The mixture was cooled to 0° C. and then EDC.HCl (42 mg, 0.22 mmol, 2.0 equiv) was added. The mixture was stirred at room temperature over weekend, concentrated, and purified by preparative. HPLC to give the title compound (36.4 mg, 61%) TFA salt as a yellow solid. MS (ES, m/z): 431.05 [M+H]⁺, ¹H NMR (400 MHz, CD₃OD) δ 7.44 (dd, J=8.6, 2.5 Hz, 1H), 7.39 (d, J=8.6 Hz, 1H), 7.27 (dd, J=7.9, 1.4 Hz, 1H), 7.13-7.07 (m, 1H), 6.90 (dd, J=8.3, 1.2 Hz, 1H), 6.77 (td, J=7.6, 1.3 Hz, 1H), 6.54 (s, 1H), 3.77 (s, 2H), 3.34 (t, J=5.7 Hz, 2H), 2.31-2.20 (m, 1H), 1.82-1.73 (m, 2H), 1.31-1.23 (m, 2H), 0.61 (dd, J=6.5, 1.8 Hz, 2H), 0.05 (s, 2H).

### Example 167

### (4-cyclopropyl-3,4-dihydroquinoxalin-1(2H)-yl)(1-((2,5-dichloro-3-hydroxybenzyl)amino)cyclopropyl)methanone

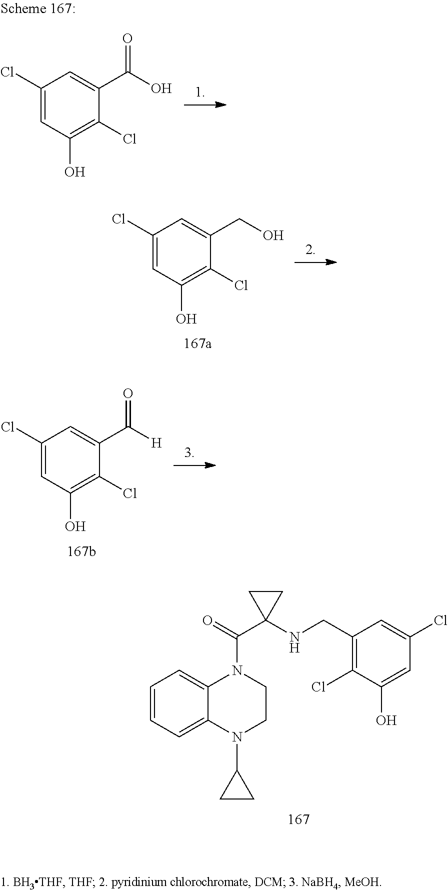 [SMILES: O=C(O)c1cc(Cl)cc(O)c1Cl ; OCc1cc(Cl)cc(O)c1Cl ; O=Cc1cc(Cl)cc(O)c1Cl ; O=C(N1CCN(C2CC2)c2ccccc21)C1(NCc2cc(Cl)cc(O)c2Cl)CC1]

Intermediate 167a: 2,5-dichloro-3-(hydroxymethyl)phenol. To a mixture of 2,5-dichloro-3-hydroxybenzoic acid (1.02 g, 4.93 mmol, 1.00 equiv) in THF (6.7 mL) at 0° C. was added borane-tetrahydrofuran complex solution (1M, 14.8 mL, 14.8 mmol, 3.00 equiv) dropwise. The mixture was stirred at 80° C. overnight. The mixture was cooled to room temperature, quenched with 2N HCl, and extracted with ethyl acetate. The organic layer was washed with 2N HCl (1×), H₂O (1×), and brine (1×), dried, concentrated, and purified by column to give 0.435 g (46%) of 2,5-dichloro-3-(hydroxymethyl)phenol as a yellow solid.

Intermediate 167b: 2,5-dichloro-3-hydroxybenzaldehyde. To a mixture of intermediate 2a (350 mg, 1.81 mmol, 1.00 equiv) in DCM (4 mL) at room temperature was added pyridinium chlorochromate (437 mg, 2.03 mmol, 1.12 equiv). The mixture was stirred at room temperature for 5h, concentrated, and purified by column to give 179 mg (52%) of 2,5-dichloro-3-hydroxybenzaldehyde as a white solid. ¹H NMR (400 MHz, CDCl₃) δ 10.34 (s, 1H), 7.49 (s, 1H), 7.28 (s, 1H), 6.07 (s, 1H).

Example 167: (4-cyclopropyl-3,4-dihydroquinoxalin-1(2H)-yl)(1-((2,5-dichloro-3-hydroxybenzyl)amino)cyclopropyl)methanone bis TFA salt. Example 167 was prepared using the procedures described in Example 165. MS (ES, m/z): 432.11 [M+H]⁺. ¹H NMR (400 MHz, CD₃OD) δ 7.30 (dd, J=7.9, 1.3 Hz, 1H), 7.24 (dd, J=8.3, 1.2 Hz, 1H), 7.19-7.13 (m, 1H), 6.89 (d, J=2.4 Hz, 1H), 6.79-6.73 (m, 1H), 6.69 (d, J=2.3 Hz, 1H), 4.04 (s, 2H), 3.89 (t, J=5.7 Hz, 2H), 3.44 (t, J=5.8 Hz, 2H), 2.46-2.39 (m, 1H), 1.37 (dd, J=7.8, 5.4 Hz, 2H), 1.21 (dd, J=7.8, 5.4 Hz, 2H), 0.86-0.79 (m, 2H), 0.58-0.49 (m, 2H).

### Example 168

### (4-cyclopropyl-3,4-dihydroquinoxalin-1(2H)-yl)(1-((3,6-dichloro-2-hydroxybenzyl)amino)cyclopropyl)methanone

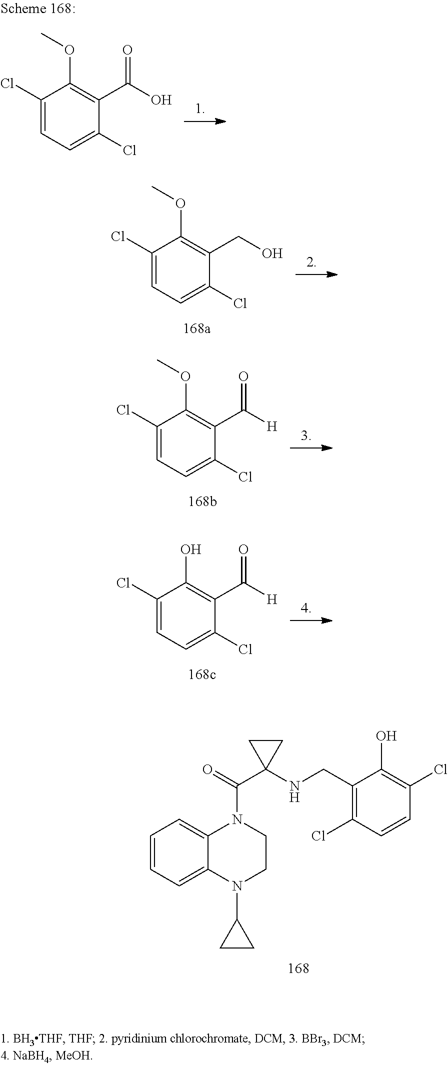

Intermediate 168a: (3,6-dichloro-2-methoxyphenyl)methanol. To a mixture of 3,6-dichloro-2-methoxybenzoic acid (1.0 g, 4.52 mmol, 1.00 equiv) in THF (6 mL) at 0° C. was added borane-tetrahydrofuran complex solution (1M, 9 mL, 9.0 mmol, 2.00 equiv) dropwise. The mixture was stirred at 80° C. overnight. The mixture was cooled to room temperature, quenched with 2N HCl, and extracted with ethyl acetate. The organic layer was washed with 2N HCl (1×), H₂O (1×), and brine (1×), dried, and concentrated to give 0.866 g (92%) of (3,6-dichloro-2-methoxyphenyl)methanol as a white solid.

Intermediate 168b: 3,6-dichloro-2-methoxybenzaldehyde. To a mixture of intermediate 168a (94.3 mg, 0.456 mmol, 1.0 equiv) in DCM (1 mL) at room temperature was added pyridinium chlorochromate (118 mg, 0.547 mmol, 1.2 equiv). The mixture was stirred at room temperature overnight, concentrated, and purified by column to give 85 mg (91%) of 3,6-dichloro-2-methoxybenzaldehyde as a white solid. ¹H NMR (400 MHz, CDCl₃) δ 10.42 (s, 1H), 7.51 (dd, J=8.7, 0.5 Hz, 1H), 7.19 (d, J=8.7 Hz, 1H), 3.96 (s, 3H).

Intermediate 168c: 3,6-dichloro-2-hydroxybenzaldehyde. To a mixture of intermediate 1b (52.6 mg, 0.255 mmol, 1.0 equiv) in DCM (3 mL) at 0° C. was added boron tribromide solution (1M, 0.77 mL, 0.77 mmol, 3 equiv). The mixture was stirred at room temperature overnight and at 45° C. for 3 h. The resulting mixture was cooled to room temperature, quenched with sat. aqu. NaHCO₃, and extracted with ethyl acetate. The organic layer was dried and concentrated to give 47 mg (96%) of 3,6-dichloro-2-hydroxybenzaldehyde as a yellow solid. ¹H NMR (400 MHz, CDCl₃) δ 12.44 (s, 1H), 10.40 (s, 1H), 7.61-7.43 (m, 1H), 7.01-6.85 (m, 1H).

Example 168: (4-cyclopropyl-3,4-dihydroquinoxalin-1(2H)-yl)(1-((3,6-dichloro-2-hydroxybenzyl)amino)cyclopropyl)methanone bis TFA salt. Example 168 was prepared using the procedures described in Example 165 substituting 167b in place of 92b. MS (ES, m/z): 432.15 [M+H]⁺. ¹H NMR (400 MHz, CD₃OD) δ 7.36 (d, J=8.7 Hz, 1H), 7.30-7.24 (m, 2H), 7.19-7.12 (m, 1H), 6.98 (d, J=8.7 Hz, 1H), 6.77-6.70 (m, 1H), 4.46 (s, 2H), 3.94 (t, J=5.7 Hz, 2H), 3.47 (t, J=5.7 Hz, 2H), 2.51-2.40 (m, 1H), 1.45-1.26 (m, 4H), 0.91-0.83 (m, 2H), 0.67-0.58 (m, 2H).

### Example 169

### (R)-(4-cyclopropyl-3,4-dihydroquinoxalin-1(2H)-yl)(3-(2,5-dichloro-3-hydroxybenzyl)thiazolidin-4-yl)methanone

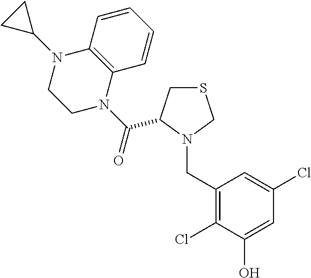

Example 169: (R)-(4-cyclopropyl-3,4-dihydroquinoxalin-1(2H)-yl)(3-(2,5-dichloro-3-hydroxybenzyl)thiazolidin-4-yl)methanone bis TFA salt. Example 169 was prepared using the procedures described in Example 162 substituting 167b in place of 92b. MS (ES, m/z): 464.10 [M+H]⁺. ¹H NMR (400 MHz, CD₃OD) δ 7.20 (dd, J=8.3, 1.3 Hz, 1H), 7.18-6.98 (m, 2H), 6.87 (s, 1H), 6.76 (s, 1H), 6.67 (t, J=7.1 Hz, 1H), 4.77 (s, 1H), 4.27 (d, J=9.6 Hz, 1H), 4.15-4.03 (m, 1H), 4.02-3.87 (m, 2H), 3.87-3.73 (m, 1H), 3.73-3.57 (m, 1H), 3.47-3.33 (m, 2H), 3.14 (s, 2H), 2.44 (s, 1H), 0.90-0.73 (m, 2H), 0.67-0.46 (m, 2H).

### Example 170

### (R)-(4-cyclopropyl-3,4-dihydroquinoxalin-1(2H)-yl)(3-(3,6-dichloro-2-hydroxybenzyl)thiazolidin-4-yl)methanone

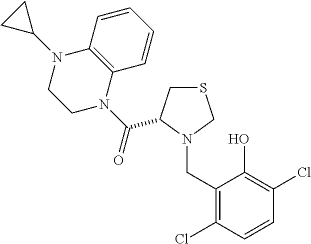

Example 170: (R)-(4-cyclopropyl-3,4-dihydroquinoxalin-1(2H)-yl)(3-(3,6-dichloro-2-hydroxybenzyl)thiazolidin-4-yl)methanone bis TFA salt. Example 170 was prepared using the procedures described in Example 162 substituting 168c in place of 92b. MS (ES, m/z): 464.06 [M+H]⁺. ¹H NMR (400 MHz, CD₃OD) δ 7.34 (d, J=8.2 Hz, 1H), 7.25-7.12 (m, 2H), 7.08-6.97 (m, 1H), 6.96-6.86 (m, 1H), 6.77-6.62 (m, 1H), 4.84-4.75 (m, 1H), 4.49 (d, J=10.0 Hz, 1H), 4.42-4.27 (m, 2H), 4.26-4.15 (m, 1H), 3.94-3.80 (m, 1H), 3.77-3.68 (m, 1H), 3.48-3.39 (m, 1H), 3.38-3.33 (m, 1H), 3.23-3.09 (m, 1H), 3.03-2.89 (m, 1H), 2.46 (s, 1H), 0.92-0.78 (m, 2H), 0.72-0.47 (m, 2H).

### Example 171

### ((4-cyclopropyl-3,4-dihydroquinoxalin-1(2H)-yl)(1-((2,5-dichloro-4-(4-(methyl((2S,3R,4R,5R)-2,3,4,5,6-pentahydroxyhexyl)amino)butyl)phenoxy)methyl)cyclopropyl)methanone

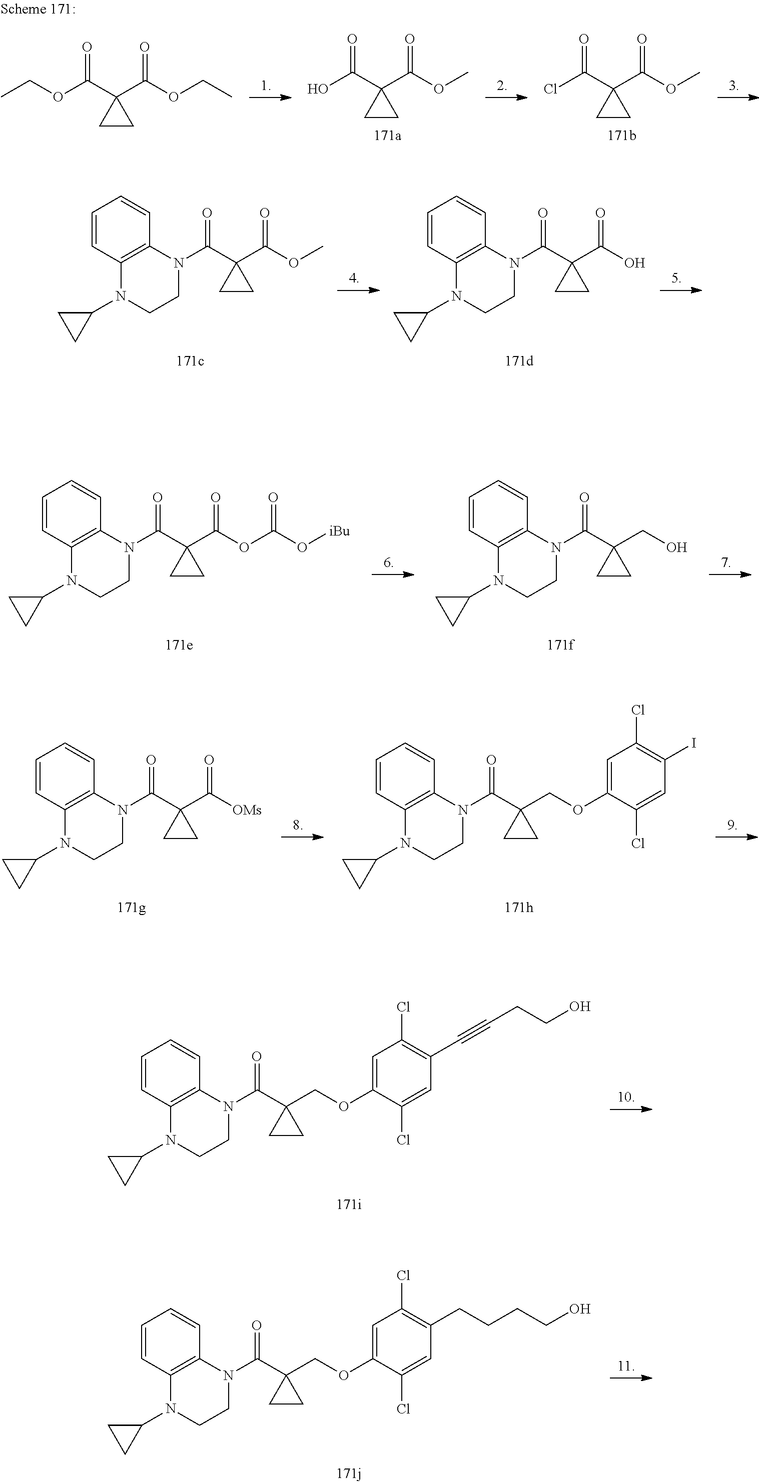

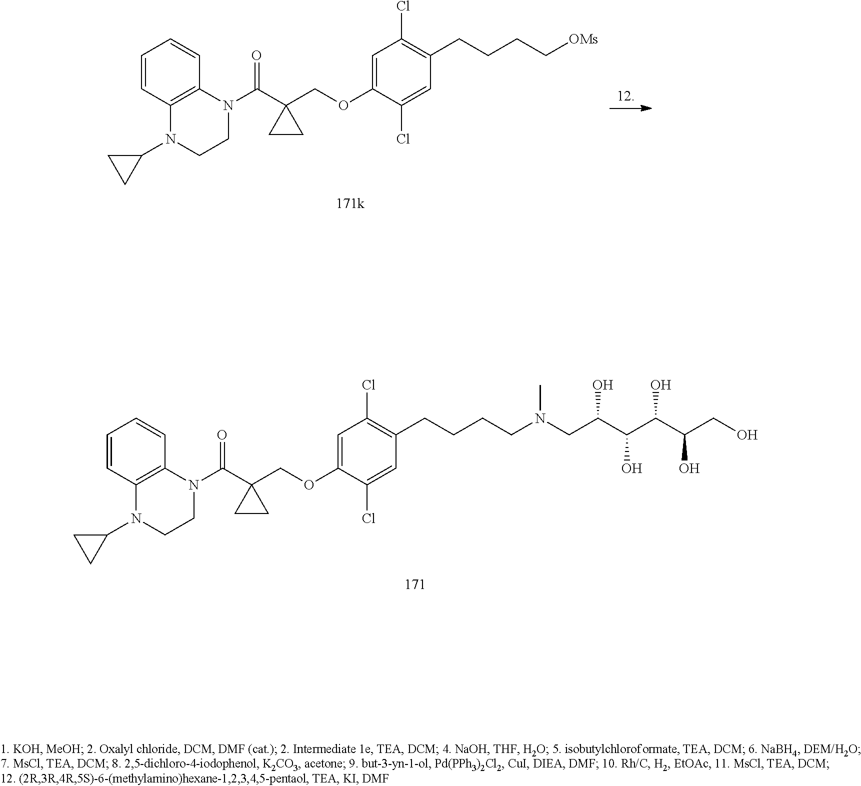 [SMILES: CS(=O)(=O)OCCCCc1cc(Cl)c(OCC2(C(=O)N3CCN(C4CC4)c4ccccc43)CC2)cc1Cl ; CN(CCCCc1cc(Cl)c(OCC2(C(=O)N3CCN(C4CC4)c4ccccc43)CC2)cc1Cl)C[C@H](O)[C@@H](O)[C@H](O)[C@H](O)CO]

Intermediate 171a: 1-(methoxycarbonyl)cyclopropanecarboxylic acid. To 1,1-diethyl cyclopropane-1,1-dicarboxylate (15.0 g, 80.56 mmol, 1.00 equiv) in methanol (90 mL) at 0° C. was added batch-wise potassium hydroxide (6.3 g, 112.28 mmol, 1.40 equiv) and the resulting solution allowed to warm to RT and stirred for 2 h. The mixture was concentrated under vacuum, diluted with 100 mL of water and then washed with 1×50 mL of ethyl acetate. The pH value of the aqueous solution was adjusted to 3-4 with conc. HCl, extracted with 3×50 mL of ethyl acetate, the organic layers combined and then washed with brine. The organic layer was dried over anhydrous sodium sulfate and concentrated to afford 9.2 g (79%) of intermediate 171a as a colorless liquid.

Intermediate 171b: methyl 1-(chlorocarbonyl)cyclopropanecarboxylate. To intermediate 171a (3.6 g, 24.98 mmol, 1.00 equiv) in DCM (15.0 mL) at 0° C. was added DMF (150 mg, 2.05 mmol, 0.10 equiv) followed by the drop-wise addition of oxalyl dichloride (4.3 g, 33.88 mmol, 1.50 equiv) and the resulting solution was allowed to warm to RT and then stirred for 1.5 h. The mixture was concentrated under vacuum to afford 3.7 g (91%) of intermediate 171b as a yellow oil, which was used without further purification.

Intermediate 171c: methyl 1-(4-cyclopropyl-1,2,3,4-tetrahydroquinoxaline-1-carbonyl)cyclopropanecarboxylate. To intermediate 1e (4.0 g, 22.96 mmol, 1.00 equiv) in dichloromethane (40.0 mL) at 0° C. was added TEA (3.5 g, 34.59 mmol, 1.50 equiv) followed by the drop-wise addition of a solution of intermediate 171b (3.7 g, 22.76 mmol, 1.00 equiv) in DCM (5.0 mL), and the resulting solution was allowed to warm to RT and then stirred for 0.5 h. The mixture was diluted with 45 mL of DCM, washed with 1×50 mL of brine, dried over anhydrous sodium sulfate and then concentrated under vacuum. The residue was purified via silica gel chromatography (ethyl acetate/petroleum ether, 1:20-1:5) to afford 6.5 g (94%) of intermediate 171c as a yellow oil.

Intermediate 171d: 1-(4-cyclopropyl-1,2,3,4-tetrahydroquinoxaline-1-carbonyl)cyclopropanecarboxylic acid. To intermediate 171c (6.5 g, 21.64 mmol, 1.00 equiv) in 1:1 THF/H₂O (50 mL) at 0° C. was added sodium hydroxide (1.7 g, 42.50 mmol, 2.00 equiv) and the resulting solution was allowed to warm to RT and then stirred overnight. The solution was adjusted to pH 2-3 with aqueous 1 M HCl and then extracted with 3×25 mL of ethyl acetate. The organic layers were combined, washed with 1×30 mL of brine, dried over anhydrous sodium sulfate and then concentrated to afford 6.0 g (97%) of intermediate 171d as a light yellow solid.

Intermediate 171e: 1-(4-cyclopropyl-1,2,3,4-tetrahydroquinoxaline-1-carbonyl)cyclopropanecarboxylic (isobutyl carbonic) anhydride. To intermediate 171d (7.0 g, 24.45 mmol, 1.00 equiv) in DCM (80.0 mL) at 0° C. was added TEA (3.6 g, 35.58 mmol, 1.50 equiv) followed by the drop-wise addition of 2-methylpropyl chloroformate (3.9 g, 28.56 mmol, 1.05 equiv) and the resulting solution was allowed to warm to RT and then stirred for 0.5 h. The mixture was diluted with 80 mL of DCM, washed with 1×50 mL of brine, dried over anhydrous sodium sulfate and then concentrated under vacuum. The residue was purified via silica gel chromatography (ethyl acetate/petroleum ether, 1:10-1:5) to afford 10.0 g (crude) of intermediate 171e as a light yellow oil.

Intermediate 171f: (4-cyclopropyl-3,4-dihydroquinoxalin-1(2H)-yl)(1-(hydroxymethyl)cyclopropyl)methanone. To intermediate 171e (10 g, 25.88 mmol, 1.00 equiv) in ethylene glycol dimethyl ether (250 mL) at −30° C. was added drop-wise a solution of NaBH₄ (2.0 g, 52.87 mmol, 2.00 equiv) in water (12.0 mL) and the resulting solution was allowed to warm to 0° C. and then stirred for 2 h. The solution was adjusted to pH 2-4 with aqueous 1 M HCl, diluted with 100 mL of water and then extracted with 3×200 mL of ethyl acetate. The organic layers were combined, dried (anhydrous sodium sulfate), concentrated and then purified via silica gel chromatography (ethyl acetate/petroleum ether, 1:10-1:1) to afford 2.5 g (35%) of intermediate 171f as a purple solid.

Intermediate 171g: (1-(4-cyclopropyl-1,2,3,4-tetrahydroquinoxaline-1-carbonyl)cyclopropyl)methyl methanesulfonate. To intermediate 171f (2.3 g, 8.45 mmol, 1.00 equiv) in DCM (45.0 mL) at 0° C. was added TEA (1.3 g, 12.85 mmol, 1.50 equiv) followed by the drop-wise addition of MsCl (1.1 g, 9.60 mmol, 1.10 equiv) and the resulting solution was allowed to warm to RT and stirred for 0.5 h. The mixture was washed with 1×30 mL of brine, dried over anhydrous sodium sulfate, concentrated and then purified via silica gel chromatography (ethyl acetate/petroleum ether, 1:20-1:1) to afford 2.7 g (91%) of intermediate 171g as a light yellow solid.

Intermediate 171h: (4-cyclopropyl-3,4-dihydroquinoxalin-1(2H)-yl)(1-((2,5-dichloro-4-iodophenoxy)methyl)cyclopropyl)methanone. To intermediate 171g (1.5 g, 4.28 mmol, 1.00 equiv) in acetone (70.0 mL) was added 2,5-dichloro-4-iodophenol (1.86 g, 6.44 mmol, 1.50 equiv) and potassium carbonate (1.18 g, 8.54 mmol, 2.00 equiv) and the reaction was stirred at 55° C. overnight. The mixture was filtered, the filtrate concentrated and the residue purified via silica gel chromatography (ethyl acetate/petroleum ether, 1:10-1:2) to afford 1.7 g (73%) of intermediate 171h as a white solid.

Intermediate 171i: (4-cyclopropyl-3,4-dihydroquinoxalin-1(2H)-yl)(1-((2,5-dichloro-4-(4-hydroxybut-1-ynyl)phenoxy)methyl)cyclopropyl)methanone. To intermediate 171h (160 mg, 0.29 mmol, 1.00 equiv) in DMF (5.0 mL) was added but-3-yn-1-ol (20.1 mg, 0.29 mmol, 1.00 equiv), DIEA (77.5 mg, 0.60 mmol, 2.00 equiv), Pd(PPh₃)₂Cl₂ (21.1 mg, 0.03 mmol, 0.10 equiv) and CuI (5.7 mg, 0.03 mmol, 0.10 equiv) and the mixture was stirred for 2 h at RT. The reaction was diluted with 20 mL of water, extracted with 3×30 mL of ethyl acetate and the organic layers combined, washed with 1×50 mL of brine and then dried over anhydrous sodium sulfate. The solution was concentrated and the residue purified via silica gel chromatography (ethyl acetate/petroleum ether, 1:10-1:2) to afford 0.18 g of intermediate 171i as a red oil.

Intermediate 171j: (4-cyclopropyl-3,4-dihydroquinoxalin-1(2H)-yl)(1-((2,5-dichloro-4-(4-hydroxybutyl)phenoxy)methyl)cyclopropyl)methanone. To intermediate 171i (300 mg, 0.62 mmol) in ethyl acetate (15.0 mL) was added Rh/C (350 mg) and the resulting suspension was stirred under a hydrogen atmosphere overnight. The mixture was diluted with 20.0 mL of methanol, filtered and the filtrate concentrated to afford 0.30 g (99%) of intermediate 171j as a red oil.

Intermediate 171k: 4-(2,5-dichloro-4-((1-(4-cyclopropyl-1,2,3,4-tetrahydroquinoxaline-1-carbonyl)cyclopropyl)methoxy)phenyl)butyl methanesulfonate. To intermediate 171j (250 mg, 0.51 mmol, 1.00 equiv) in DCM (10.0 mL) at 0° C. was added TEA (77.4 mg, 0.76 mmol, 1.50 equiv) followed by the drop-wise addition of MsCl (70.2 mg, 0.61 mmol, 1.20 equiv) and the resulting solution allowed to warm to RT and then stirred for 0.5 h. The mixture was diluted with 20.0 mL of DCM, washed with 1×20 mL of brine, dried over anhydrous sodium sulfate and then concentrated to afford 260 mg (90%) of intermediate 171k as a purple oil.

Example 171: (4-cyclopropyl-3,4-dihydroquinoxalin-1(2H)-yl)(1-((2,5-dichloro-4-(4-(methyl((2S,3R,4R,5R)-2,3,4,5,6-pentahydroxyhexyl)amino)butyl)phenoxy)methyl)-cyclopropyl)methanone. To intermediate 171k (320 mg, 0.56 mmol, 1.00 equiv) in DMF (7.0 mL) was added (2R,3R,4R,5S)-6-(methylamino)hexane-1,2,3,4,5-pentaol (160 mg, 0.82 mmol, 1.50 equiv), TEA (85.0 mg, 0.84 mmol, 1.50 equiv) and KI (93.6 mg, 0.56 mmol, 1.00 equiv) and the reaction was stirred overnight at 75° C. The mixture was then filtered, concentrated and the residue purified by reverse-phase (C18) prep-HPLC to afford 24.6 mg (7%) of Example 171 trifluoroacetic acid salt as an off-white solid. LCMS (ES, m/z): 666 [M+H]⁺. ¹H NMR (300 MHz, CD₃OD, ppm): 7.41-7.43 (d, J=7.5 Hz, 1H), 7.35 (s, 1H), 7.10-7.12 (m, 2H), 6.54-6.72 (m, 2H), 4.10-4.18 (m, 1H), 3.88-3.92 (m, 2H), 3.81-3.83 (m, 2H), 3.66-3.79 (m, 5H), 3.41-3.433 (m, 2H), 3.26-3.29 (m, 4H), 2.92-2.94 (m, 2H), 2.72-2.77 (m, 2H), 2.28 (m, 1H), 1.68-1.70 (m, 4H), 1.35-1.38 (m, 2H), 0.96-1.00 (m, 2H), 0.67-0.69 (m, 2H), 0.19-0.21 (m, 2H).

### Example 172

### 5-(3-((1-(4-cyclopropyl-1,2,3,4-tetrahydroquinoxaline-1-carbonyl)cyclopropylamino)methyl)-4-(trifluoromethyl)phenyl)-N-methyl-N-((2S,3R,4R,5R)-2,3,4,5,6-pentahydroxyhexyl)pentanamide

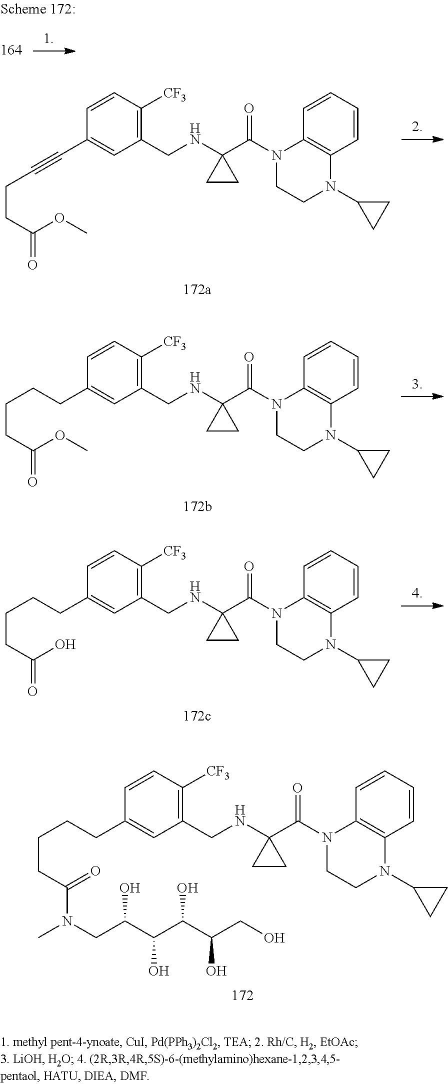 [SMILES: COC(=O)CCC#Cc1ccc(C(F)(F)F)c(CNC2(C(=O)N3CCN(C4CC4)c4ccccc43)CC2)c1 ; COC(=O)CCCCc1ccc(C(F)(F)F)c(CNC2(C(=O)N3CCN(C4CC4)c4ccccc43)CC2)c1 ; O=C(O)CCCCc1ccc(C(F)(F)F)c(CNC2(C(=O)N3CCN(C4CC4)c4ccccc43)CC2)c1 ; CN(C[C@H](O)[C@@H](O)[C@H](O)[C@H](O)CO)C(=O)CCCCc1ccc(C(F)(F)F)c(CNC2(C(=O)N3CCN(C4CC4)c4ccccc43)CC2)c1]

Intermediate 172a: methyl 5-(3-((1-(4-cyclopropyl-1,2,3,4-tetrahydroquinoxaline-1-carbonyl)cyclopropylamino)methyl)-4-(trifluoromethyl)phenyl)pent-4-ynoate. To Example 164 (500 mg, 0.92 mmol, 1.00 equiv) in DMF (6 mL) was added methyl pent-4-ynoate (155 mg, 1.38 mmol, 1.50 equiv), Pd(PPh₃)₂Cl₂ (324 mg, 0.46 mmol, 0.50 equiv), CuI (175 mg, 0.92 mmol, 0.99 equiv) and triethylamine (186 mg, 1.84 mmol, 1.99 equiv) and the reaction was stirred for 2 h. The mixture was diluted with 50 mL of ethyl acetate, washed with 2×50 mL of brine, dried over anhydrous sodium sulfate and concentrated under vacuum. The residue was purified via silica gel chromatography (ethyl acetate/petroleum ether, 1:5) to afford 489 mg (100%) of intermediate 172a as a yellow oil.

Intermediate 172b: methyl 5-(3-((1-(4-cyclopropyl-1,2,3,4-tetrahydroquinoxaline-1-carbonyl)cyclopropylamino)methyl)-4-(trifluoromethyl)phenyl)pentanoate. To intermediate 172a (489 mg, 0.93 mmol, 1.00 equiv) in ethyl acetate (40 mL) was added Rh/C (734 mg) and the suspension was stirred under a hydrogen atmosphere at 30° C. for 2 days. The solids were filtered out and the filtrate concentrated to afford 511 mg of intermediate 172b as a yellow oil.

Intermediate 172c: 5-(3-((1-(4-cyclopropyl-1,2,3,4-tetrahydroquinoxaline-1-carbonyl)cyclopropylamino)methyl)-4-(trifluoromethyl)phenyl)pentanoic acid. To intermediate 172b (511 mg, 0.96 mmol, 1.00 equiv) in THF/H₂O (20/10 mL) was added LiOH.H₂O (406 mg, 9.68 mmol, 10.02 equiv) and the reaction was stirred for 3 h. The solution was adjusted to pH 3-4 with aqueous 6 M HCl and then extracted with 2×50 mL of ethyl acetate. The organic layers were combined, washed with 2×50 mL of brine, dried over anhydrous sodium sulfate and then concentrated to afford 420 mg (84%) of intermediate 172cas a brown oil.

Example 172: 5-(3-((1-(4-cyclopropyl-1,2,3,4-tetrahydroquinoxaline-1-carbonyl)cyclopropylamino)methyl)-4-(trifluoromethyl)phenyl)-N-methyl-N-((2S,3R,4R,5R)-2,3,4,5,6-pentahydroxyhexyl)pentanamide. To intermediate 172c (220 mg, 0.43 mmol, 1.00 equiv) in DMF (6 mL) was added (2R,3R,4R,5S)-6-(methylamino)hexane-1,2,3,4,5-pentol (84 mg, 0.43 mmol, 1.00 equiv), HATU (195 mg, 0.51 mmol, 1.20 equiv) and DIEA (66 mg, 0.51 mmol, 1.20 equiv) and the reaction stirred for 1 h. The mixture was diluted with 50 mL of ethyl acetate, washed with 2×50 mL of brine, dried over anhydrous sodium sulfate, concentrated and then purified by reverse-phase (C18) Prep-HPLC to afford 67.2 mg (23%) of Example 172 as a green solid. LCMS (ES, m/z): 693 [M+H]⁺. ¹H NMR (300 MHz, CD₃OD, ppm): 7.58 (d, J=8.1 Hz, 1H), 7.34-7.21 (m, 4H), 7.96 (d, J=6.3 Hz, 1H), 6.80 (d, J=7.2, 1H), 4.03-3.92 (m, 3H), 3.90 (d, J=5.7 Hz, 2H), 3.77-3.65 (m, 7H), 3.43 (d, J=6.0 Hz, 3H), 3.18-3.15 (m, 2H), 3.01-2.98 (m, 2H), 2.67-2.56 (m, 3H), 2.46-2.42 (m, 2H), 1.65 (s, 4H), 1.43-1.39 (m, 2H), 1.18-1.14 (m, 2H), 0.82-0.80 (m, 2H), 0.46 (s, 2H).

### Example 173

### 2-({4-[bis(2-hydroxyethyl)aminol-6-{[5-(2,5-dichloro-4-{1(4R)-4-[(4-cyclopropyl-1,2,3,4-tetrahydroquinoxalin-1-yl)carbonyl]-1,3-thiazolidin-3-yl]methyl}phenyl)pentyl]amino}-1,3,5-triazin-2-yl}(2-hydroxyethyl)amino)ethan-1-ol

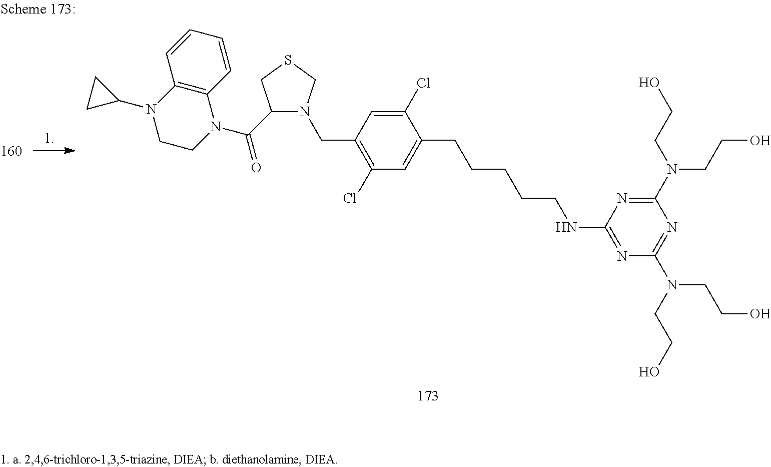 [SMILES: O=C(C1CSCN1Cc1cc(Cl)c(CCCCCNc2nc(N(CCO)CCO)nc(N(CCO)CCO)n2)cc1Cl)N1CCN(C2CC2)c2ccccc21]

Example 173: 2-({4-[bis(2-hydroxyethyl)amino]-6-{[5-(2,5-dichloro-4-{[(4R)-4-[(4-cyclopropyl-1,2,3,4-tetrahydroquinoxalin-1-yl)carbonyl]-1,3-thiazolidin-3-yl]methyl}phenyl)pentyl]amino}-1,3,5-triazin-2-yl}(2-hydroxyethyl)amino)ethan-1-ol. To 2,4,6-trichloro-1,3,5-triazine (17 mg, 0.094 mmol) in THF (0.5 mL) at 0° C. was added a 0° C. solution of 160 (50 mg, 0.094 mmol) in THF (0.5 mL) followed by DIEA (48 μl, 0.28 mmol) and the solution stirred for 30 min at 0° C. The mixture was allowed to warm to room temperature and then stirred and additional 30 min. The solvent was then removed and the resulting residue dissolved in DMF (1 mL), then DIEA (48 μl, 0.28 mmol) and diethanolamine (39 mg, 0.28 mmol) were added, and the resulting mixture was stirred at 60° C. for 6 h. The mixture was then diluted with H₂O, acidified with TFA, and then purified by preparative HPLC with a C18 silica gel stationary phase using a gradient of H₂O 0.05% TFA: CH₃CN 0.05% TFA (70:30 to 5:95) and detection by UV at 254 nm to give the title compound (23 mg, 21%) tri-TFA salt. MS (ES, m/z): 818.26 [M+H]⁺; ¹H NMR (400 MHz, CD₃OD) δ 7.30-6.98 (m, 5H), 6.64 (t, J=7.6 Hz, 1H), 4.76-4.62 (m, 1H), 4.10 (d, J=9.3 Hz, 2H), 3.98-3.61 (m, 12H), 3.60-3.33 (m, 6H), 3.27-3.05 (m, 8H), 2.72 (t, J=7.5 Hz, 2H), 2.46-2.31 (m, 1H), 1.72-1.59 (m, 4H), 1.49-1.39 (m, 2H), 0.81 (d, J=6.5 Hz, 2H), 0.61-0.41 (m, 2H).

### Example 174

### 2-(3-(2,5-dichloro-4-(((R)-4-(4-cyclopropyl-1,2,3,4-tetrahydroquinoxaline-1-carbonyl)thiazolidin-3-yl)methyl)phenyl)propyl)-N1,N3-bis((2R,3S,4S,5S)-2,3,4,5,6-pentahydroxyhexyl)malonamide

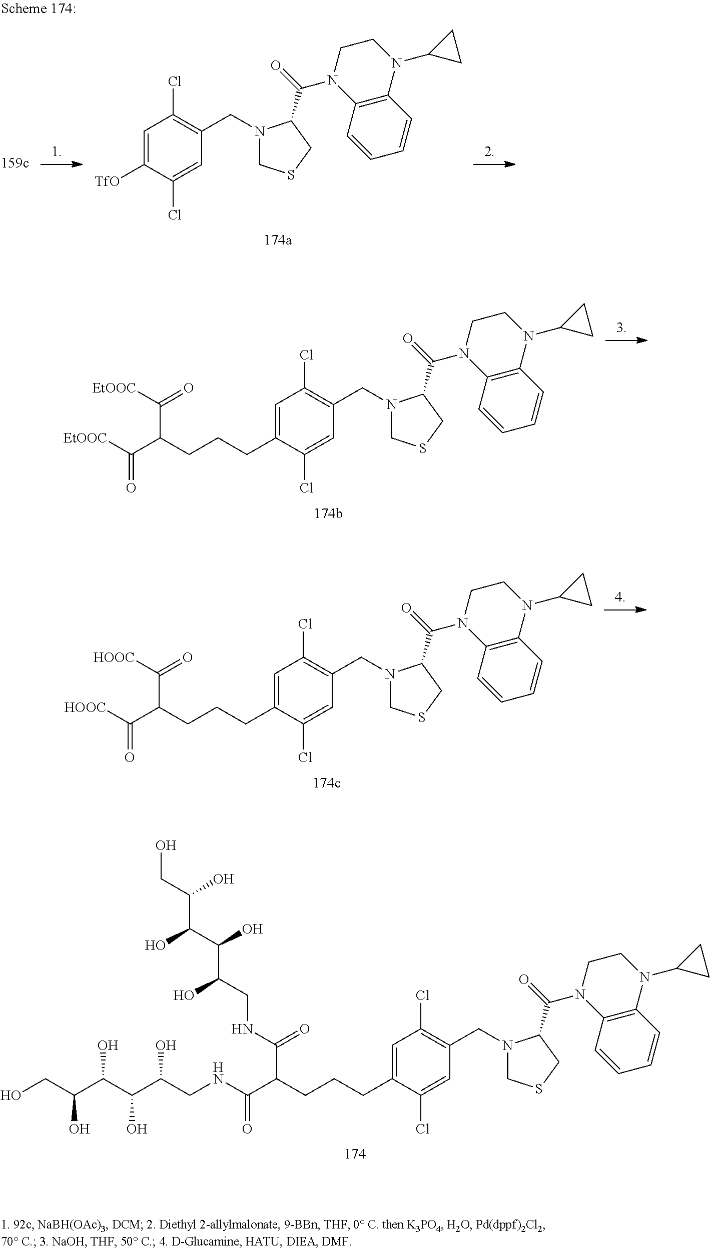 [SMILES: O=C([C@@H]1CSCN1Cc1cc(Cl)c(OS(=O)(=O)C(F)(F)F)cc1Cl)N1CCN(C2CC2)c2ccccc21 ; CCOC(=O)C(=O)C(CCCc1cc(Cl)c(CN2CSC[C@H]2C(=O)N2CCN(C3CC3)c3ccccc32)cc1Cl)C(=O)C(=O)OCC ; O=C(O)C(=O)C(CCCc1cc(Cl)c(CN2CSC[C@H]2C(=O)N2CCN(C3CC3)c3ccccc32)cc1Cl)C(=O)C(=O)O ; O=C(NC[C@@H](O)[C@H](O)[C@@H](O)[C@@H](O)CO)C(CCCc1cc(Cl)c(CN2CSC[C@H]2C(=O)N2CCN(C3CC3)c3ccccc32)cc1Cl)C(=O)NC[C@@H](O)[C@H](O)[C@@H](O)[C@@H](O)CO]

Intermediate 174a (R)-2,5-dichloro-4-((4-(4-cyclopropyl-1,2,3,4-tetrahydroquinoxaline-1-carbonyl)thiazolidin-3-yl)methyl)phenyl trifluoromethanesulfonate: To a solution of 159c (335 mg, 1.03 mmol, 1.2 equiv) and 92c, (250 mg, 0.87 mmol, 1.0 equiv) in dichloromethane (1.7 mL) was added NaBH(OAc)₃ (275 mg, 1.29 mmol, 1.5 equiv). The resulting solution was stirred at room temperature for 18 hours and then quenched with aqueous NaHCO₃, diluted with dichloromethane (50 mL) and washed with water (4×50 mL) and brine (50 mL). The combined organic layers were dried over anhydrous sodium sulfate and concentrated under vacuum. The residue was purified by silica gel column chromatography with an eluent gradient of hexane:ethyl acetate (100:1 to 5:1) to furnish 174a (324 mg, 64%). MS (ES, m/z): 596.0 [M+H]⁺.

Intermediate 174b (R)-diethyl 3-(3-(2,5-dichloro-4-((4-(4-cyclopropyl-1,2,3,4-tetrahydroquinoxaline-1-carbonyl)thiazolidin-3-yl)methyl)phenyl)propyl)-2,4-dioxopentanedioate: To diethyl 2-allylmalonate (200 mg, 1 mmol, 1.0 equiv) in dry tetrahydrofuran (0.6 mL) at 0° C. was added dropwise a solution of 9-BBn (0.5M in THF, 2 mL, 1.0 equiv) over 3 minutes. The ice-bath was removed and the reaction mixture stirred overnight. Aqueous K₃PO₄ (636 mg in 0.7 mL H₂O, 3.0 equiv) was added dropwise. Half of the resulting solution (1.3 mL, 2.8 equiv) was added to (R)-2,5-dichloro-4-((4-(4-cyclopropyl-1,2,3,4-tetrahydroquinoxaline-1-carbonyl)thiazolidin-3-yl)methyl)phenyl trifluoromethanesulfonate (108 mg, 0.18 mmol, 1.0 equiv), the mixture was purged with N₂ (3×N₂/vacuum cycles), and then Pd(dppf)Cl₂ (13.2 mg, 0.09 equiv) was added. The mixture was again purged with N₂ (3×N₂/vacuum cycles) and then heated to 70° C. under an inert atmosphere. After 2 hours additional alkyl borate solution (0.5 mL, 0.15 mmol, 1.0 equiv) and Pd(dppf)Cl₂ (2.0 mg, 0.013 mmol) were added. After an additional hour the reaction mixture was cooled, diluted with ethyl acetate (50 mL) and washed with water (3×50 mL) and brine (50 mL). The combined organic layers were dried over anhydrous sodium sulfate and concentrated under vacuum. The residue was purified by silica gel column chromatography with an eluent gradient of hexane:ethyl acetate (100:1 to 5:1) to furnish 174b (50 mg, 43%). MS (ES, m/z): 704.3 [M+H]⁺.

Intermediate 174c (R)-3-(3-(2,5-dichloro-4-((4-(4-cyclopropyl-1,2,3,4-tetrahydroquinoxaline-1-carbonyl)thiazolidin-3-yl)methyl)phenyl)propyl)-2,4-dioxopentanedioic acid: To a solution of (R)-diethyl 3-(3-(2,5-dichloro-4-((4-(4-cyclopropyl-1,2,3,4-tetrahydroquinoxaline-1-carbonyl)thiazolidin-3-yl)methyl)phenyl)propyl)-2,4-dioxopentanedioate (50 mg, 0.077 mmol, 1.0 equiv) in tetrahydrofuran (0.5 mL) was added aqueous NaOH (3M, 0.39 mmol, 0.128 mL, 5.0 equiv) and the resulting mixture was stirred vigorously overnight at 50° C. The reaction was diluted with ethyl acetate (5 mL) and water (5 mL) and the pH was adjusted to 3 with aqueous HCl (1M). The aqueous layer was extracted with ethyl acetate (3×5 mL) and the combined organic layers washed with brine (10 mL). The resulting mixture was dried over anhydrous sodium sulfate and concentrated over vacuum to afford 174c (30 mg, 60%) which was used without further purification. MS (ES, m/z): 648.2 [M+H]⁺.

Example 174 (2-(3-(2,5-dichloro-4-(((R)-4-(4-cyclopropyl-1,2,3,4-tetrahydroquinoxaline-1-carbonyl)thiazolidin-3-yl)methyl)phenyl)propyl)-N1,N3-bis((2R,3 S,4S,5S)-2,3,4,5,6-pentahydroxyhexyl)malonamide TFA salt. To a solution of 174c (30 mg, 0.05 mmol, 1.0 equiv), D-Glucamine (20 mg, 0.11 mmol, 2.2 equiv) and DIEA (12.9 mg, 0.1 mmol, 2.0 equiv) in DMF (0.5 mL) was added dropwise HATU (38.2 mg, 0.1 mmol, 2.0 equiv) in DMF (0.5 mL) and the reaction mixture was stirred at room temperature for 5 minutes. The crude solution was diluted with DMF:H₂O (1:1) to 4 mL, acidified with TFA, and purified by preparative HPLC with a C18 silica gel stationary phase using a gradient of H₂O 0.05% TFA: CH₃CN 0.05% TFA (90:10 to 10:90) over 30 min and detection by UV at 254 nm to give 7.7 mg (13%) of the title compound as a white solid. MS (ES, m/z): 918.5 [M+H]⁺.

### Example 175

### 2-((2,5-dichloro-4-(((R)-4-(4-cyclopropyl-1,2,3,4-tetrahydroquinoxaline-1-carbonyl)thiazolidin-3-yl)methyl)benzyl)thio)-1-methyl-N-((2S,3R,4R,5R)-2,3,4,5,6-pentahydroxyhexyl)-1H-imidazole-5-carboxamide

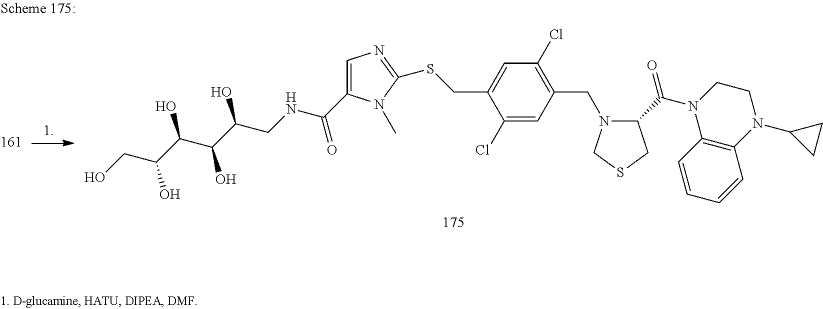 [SMILES: Cn1c(C(=O)NC[C@H](O)[C@@H](O)[C@H](O)[C@H](O)CO)cnc1SCc1cc(Cl)c(CN2CSC[C@H]2C(=O)N2CCN(C3CC3)c3ccccc32)cc1Cl]

Example 175: 2-((2,5-dichloro-4-(((R)-4-(4-cyclopropyl-1,2,3,4-tetrahydroquinoxaline-1-carbonyl)thiazolidin-3-yl)methyl)benzyl)thio)-1-methyl-N-((2 S,3R,4R,5R)-2,3,4,5,6-pentahydroxyhexyl)-1H-imidazole-5-carboxamide. To a mixture of example 161 (23.4 mg, 0.038 mmol, 1 equiv) and D-glucamine (8.2 mg, 0.0454 mmol, 1.2 equiv) in DMF (0.2 mL) were added HATU (17.3 mg, 0.045 mmol, 1.2 equiv) and DIPEA (33 uL, 0.19 mmol, 5 equiv). The mixture was stirred at rt for 30 minutes and then purified by prep-HPLC to give 13.3 mg (31%) of example 175 bis TFA salt as a white solid. ¹H NMR (400 MHz, CD₃OD) δ 7.95 (s, 1H), 7.20 (dd, J=8.3, 1.3 Hz, 2H), 7.17-7.13 (m, 1H), 7.13-6.99 (m, 2H), 6.64 (td, J=7.7, 1.2 Hz, 1H), 4.81-4.69 (m, 1H), 4.35 (s, 2H), 4.20-4.08 (m, 1H), 4.05 (d, J=9.8 Hz, 1H), 3.98-3.89 (m, 1H), 3.88-3.81 (m, 1H), 3.82-3.75 (m, 6H), 3.74-3.69 (m, 1H), 3.68 (d, J=1.9 Hz, 1H), 3.67-3.62 (m, 1H), 3.62-3.59 (m, 1H), 3.59-3.55 (m, 1H), 3.52-3.33 (m, 5H), 3.18-3.01 (m, 1H), 2.48-2.38 (m, 1H), 0.90-0.76 (m, 2H), 0.65-0.38 (m, 2H). LCMS (ES, m/z): 781.24 [M+H]⁻.

### Example 176

### 4-(2,5-dichloro-4-{[(4R)-4-[(4-cyclopropyl-1,2,3,4-tetrahydroquinoxalin-1-yl)carbonyl]-1,3-thiazolidin-3-yl]methyl}phenoxymethyl)-N-[(2S,3R,4R,5R)-2,3,4,5,6-pentahydroxyhexyl]benzamide

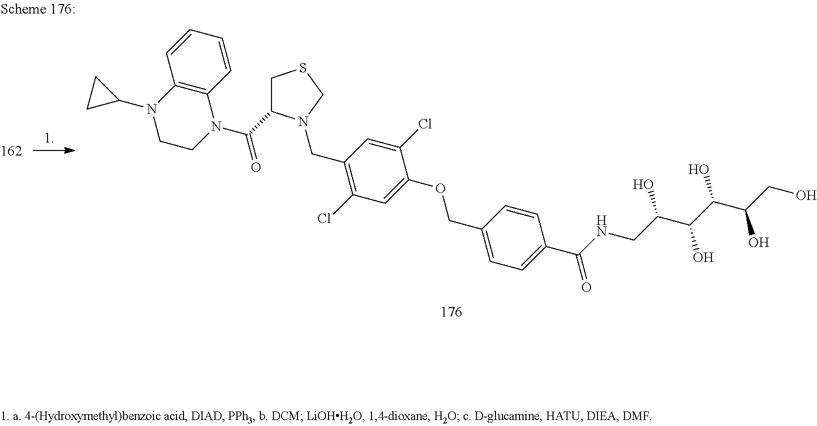 [SMILES: O=C(NC[C@H](O)[C@@H](O)[C@H](O)[C@H](O)CO)c1ccc(COc2cc(Cl)c(CN3CSC[C@H]3C(=O)N3CCN(C4CC4)c4ccccc43)cc2Cl)cc1]

Example 176: 4-(2,5-dichloro-4-{[(4R)-4-[(4-cyclopropyl-1,2,3,4-tetrahydroquinoxalin-1-yl)carbonyl]-1,3-thiazolidin-3-yl]methyl}phenoxymethyl)-N-[(2S,3R,4R,5R)-2,3,4,5,6-pentahydroxyhexyl]benzamide. To Example 162 (172 mg, 0.370 mmol), methyl 4-(hydroxymethyl)benzoic acid (77 mg, 0.46 mmol), and triphenyphosphine (121 mg, 0.46 mmol) in DCM (3 mL) at 0° C. was added diisopropyl azodicarboxylate (91 μL, 0.46 mmol) and the mixture was allowed to warm to room temperature and then stirred for 16 h. The solvent was removed and the residue dissolved in a mixture of H₂O (5 mL) and 1,4-dioxane (25 mL). To this was added LiOH.H₂O (62 mg, 1.5 mmol) and the mixture stirred at room temperature for 2 h. The solvent was removed, the residue dissolved in DCM and then washed with 1M aqueous HCl, dried over Na₂SO₄, then filtered and concentrated. To a portion of the crude residue (˜0.123 mmol), D-glucamine (42 mg, 0.23 mmol), and DIEA (128 μL, 0.740 mmol) in DMF (2 mL) was added HATU (70 mg, 0.19 mmol) and the reaction stirred for 1 h. The mixture was diluted with H₂O, acidified with TFA, and then purified by preparative HPLC with a C18 silica gel stationary phase using a gradient of H₂O 0.05% TFA:CH₃CN 0.05% TFA (70:30 to 5:95) and detection by UV at 254 nm to give the title compound (21 mg, 17%) as a bis-TFA salt. MS (ES, m/z): 761.34 [M+H]⁺; ¹H NMR (400 MHz, CD₃OD) δ 7.88 (d, J=8.0 Hz, 2H), 7.58 (d, J=7.1 Hz, 2H), 7.25 (s, 1H), 7.20 (dd, J=8.3, 1.3 Hz, 1H), 7.17-7.08 (m, 2H), 7.08-6.99 (m, 1H), 6.67 (t, J=7.2 Hz, 1H), 5.25 (s, 2H), 4.80-4.72 (m, 1H), 4.29 (d, J=9.9 Hz, 1H), 4.14-3.86 (m, 4H), 3.86-3.76 (m, 3H), 3.76-3.61 (m, 5H), 3.61-3.51 (m, 1H), 3.47 (dd, J=13.7, 7.3 Hz, 1H), 3.43-3.35 (m, 1H), 3.21-3.09 (m, 2H), 2.44 (s, 1H), 0.80 (s, 2H), 0.56 (s, 2H).

### Example 177

### 1-(4-(2,5-dichloro-4-(((R)-4-(4-cyclopropyl-1,2,3,4-tetrahydroquinoxaline-1-carbonyl)thiazolidin-3-yl)methyl)phenyl)butyl)-3-((2R,3S,4S,5S)-2,3,4,5,6-pentahydroxyhexyl)urea

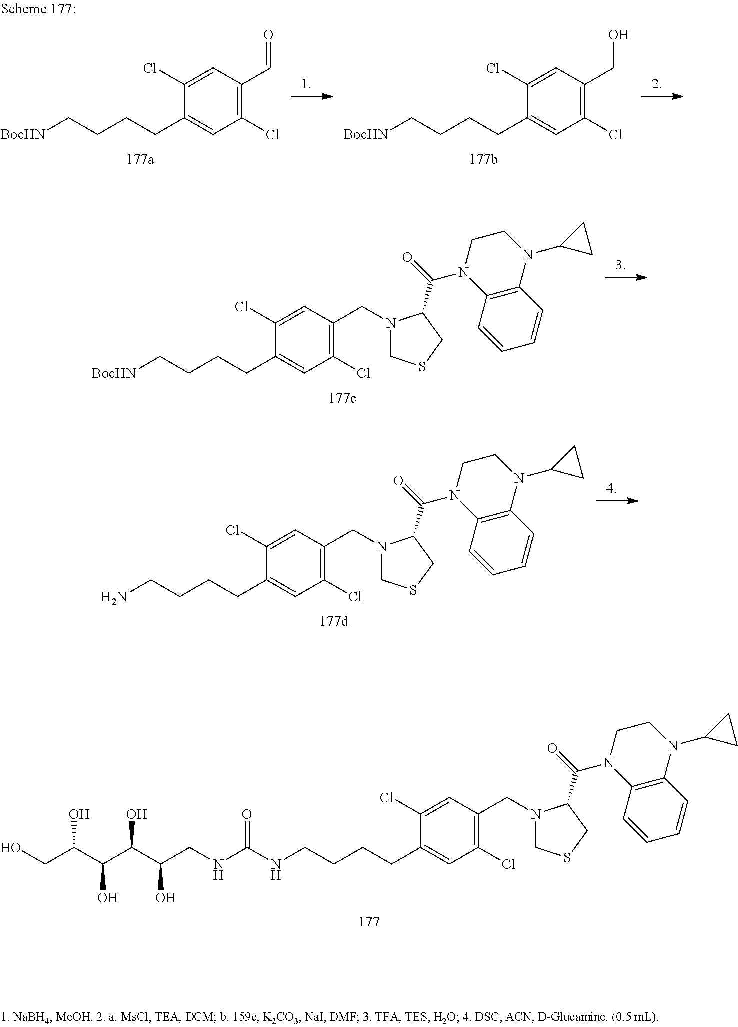 [SMILES: CC(C)(C)OC(=O)NCCCCc1cc(Cl)c(C=O)cc1Cl ; CC(C)(C)OC(=O)NCCCCc1cc(Cl)c(CO)cc1Cl ; CC(C)(C)OC(=O)NCCCCc1cc(Cl)c(CN2CSC[C@H]2C(=O)N2CCN(C3CC3)c3ccccc32)cc1Cl ; NCCCCc1cc(Cl)c(CN2CSC[C@H]2C(=O)N2CCN(C3CC3)c3ccccc32)cc1Cl ; O=C(NCCCCc1cc(Cl)c(CN2CSC[C@H]2C(=O)N2CCN(C3CC3)c3ccccc32)cc1Cl)NC[C@@H](O)[C@H](O)[C@@H](O)[C@@H](O)CO]

Intermediate 177a: tert-butyl 4-(2,5-dichloro-4-formylphenyl)butylcarbamate. Tert-butyl 4-(2,5-dichloro-4-formylphenyl)butylcarbamate was prepared using the procedures described in the synthesis of intermediate 160d, substituting but-3-yn-1-amine for pent-4-yn-1-amine, to afford 177a (2.11g) as a brown oil.

Intermediate 177b: tert-butyl N-[4-[2,5-dichloro-4-(hydroxymethyl)phenyl]butyl]carbamate. To tert-butyl N-[4-(2,5-dichloro-4-formylphenyl)butyl]carbamate (2.11 g, 6.09 mmol, 1.00 equiv) in methanol (30 mL) at 0-5° C. was added NaBH₄ (460 mg, 12.16 mmol, 2.00 equiv) in several batches over a period of 1 h. The reaction was stirred for 1 h at 0-5° C. and then quenched by the addition of 50 mL of water. The resulting mixture was concentrated under vacuum and extracted with dichloromethane (3×100 mL). The combined organic layers were washed with brine (3×100 mL), dried over anhydrous sodium sulfate and concentrated under vacuum to afford 1.70 g (80%) of 177b as brown oil. ¹H-NMR (300 MHz, CDCl₃): 7.45 (s, 1H), 7.17 (s, 1H), 4.69 (s, 2H), 4.69 (s, 0.64H), 3.13-3.11 (m, 2H), 2.70-2.65 (m, 2H), 1.93 (s, 0.6H), 1.62-1.45 (m, 4H), 1.41 (s, 8.9H).

Intermediate 177c: (R)-tert-butyl 4-(2,5-dichloro-4-((4-(4-cyclopropyl-1,2,3,4-tetrahydroquinoxaline-1-carbonyl)thiazolidin-3-yl)methyl)phenyl)butylcarbamate. To a solution of tert-butyl N-[4-[2,5-dichloro-4-(hydroxymethyl)phenyl]butyl]carbamate (150 mg, 0.43 mmol, 1.0 equiv) and triethylamine (87 mg, 0.86 mmol, 2.0 equiv) in dichloromethane (0.8 mL) at 0° C. was added methanesulfonyl chloride (49 mg, 0.43 mmol, 1.0 equiv) and the reaction stirred for 20 minutes. The solvent was removed, the residue dissolved in DMF (0.5 mL), and the mixture was then added to a solution of 159c (124 mg, 0.43 mmol, 1.0 equiv) and K₂CO₃ (65 mg, 0.47 mmol, 1.1 equiv) in DMF (0.5 mL). NaI (6.4 mg, 0.1 equiv) was then added and the solution heated to 70° C. overnight. The reaction was diluted with ethyl acetate (40 mL), washed with water (3×30 mL) and brine (30 mL), and dried over anhydrous sodium sulfate. The solution was concentrated by vacuum and the residue purified by silica gel column chromatography with an eluent gradient of hexane:ethyl acetate (100:1 to 4:1) to furnish 177c (32.9 mg, 12%) as a yellow oil. MS (ES, m/z): 619.1 [M+H]⁺.

Intermediate 177d: (R)-(3-(4-(4-aminobutyl)-2,5-dichlorobenzyl)thiazolidin-4-yl)(4-cyclopropyl-3,4-dihydroquinoxalin-1(2H)-yl)methanone. To (R)-tert-butyl 4-(2,5-dichloro-4-((4-(4-cyclopropyl-1,2,3,4-tetrahydroquinoxaline-1-carbonyl)thiazolidin-3-yl)methyl)phenyl)butylcarbamate (33 mg, 0.05 mmol, 1.0 equiv) was added a solution of trifluoroacetic acid:triethylsilane:water (95:5:5) and the reaction stirred for 5 minutes. The mixture was quenched with aqueous NaHCO₃ (˜20 mL), extracted with dichloromethane (2×20 mL) and the organic layer was concentrated to afford 177d (30.5 mg, 100%) which was used without further purification. MS (ES, m/z): 519.1 [M+H]⁺.

Example 177: 1-(4-(2,5-dichloro-4-(((R)-4-(4-cyclopropyl-1,2,3,4-tetrahydroquinoxaline-1-carbonyl)thiazolidin-3-yl)methyl)phenyl)butyl)-3-((2R,3S,4S,5S)-2,3,4,5,6-pentahydroxyhexyl)urea. To a solution of 177d (30.5 mg, 0.6 mmol, 1.0 equiv) in acetonitrile (0.5 mL) was added N—N′-disuccinimidyl carbonate (16.5 mg, 0.65 mmol, 1.1 equiv). After 30 minutes, D-glucamine (16 mg, 0.09 mmol, 1.5 equiv) and DMF (0.3 mL) were added and the reaction mixture was stirred at 80° C. for 90 minutes. The resulting mixture was diluted to 4 mL with acetonitrile:water (1:1), acidified with TFA, and then purified by preparative HPLC with a C18 silica gel stationary phase using a gradient of H₂O 0.05% TFA: CH₃CN 0.05% TFA (90:10 to 5:95) and detection by UV at 254 nm to give the title compound (16.7 mg, 29%) as the TFA salt. MS (ES, m/z): 726.3 [M+H]⁺.

### Example 178

### 5-(2,5-dichloro-4-(((R)-4-(4-cyclopropyl-1,2,3,4-tetrahydroquinoxaline-1-carbonyl)thiazolidin-3-yl)methyl)phenyl)-N-((2S,3R,4R,5R)-2,3,4,5,6-pentahydroxyhexyl)pentanamide

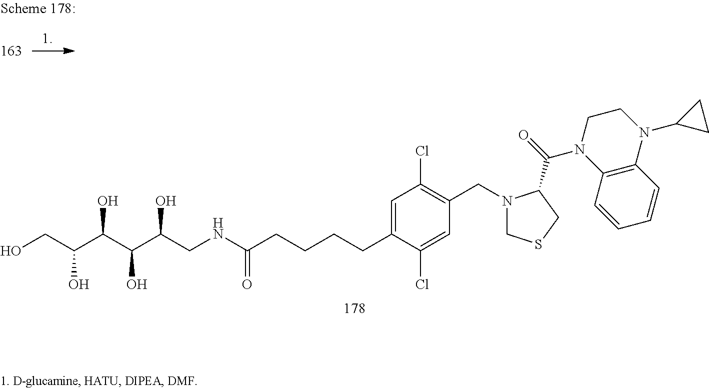 [SMILES: O=C(CCCCc1cc(Cl)c(CN2CSC[C@H]2C(=O)N2CCN(C3CC3)c3ccccc32)cc1Cl)NC[C@H](O)[C@@H](O)[C@H](O)[C@H](O)CO]

Example 178: 5-(2,5-dichloro-4-(((R)-4-(4-cyclopropyl-1,2,3,4-tetrahydroquinoxaline-1-carbonyl)thiazolidin-3-yl)methyl)phenyl)-N-((2S,3R,4R,5R)-2,3,4,5,6-pentahydroxyhexyl)pentanamide. To a mixture of intermediate 163 (11.4 mg, 0.0193 mmol, 1 equiv) and D-glucamine (4.2 mg, 0.0232 mmol, 1.2 equiv) in DMF (0.14 mL) was added HATU (8.8 mg, 0.232 mmol, 1.2 equiv) and DIPEA (13.4 μL, 0.77 mmol, 4 equiv). The mixture was stirred at rt for 1 h and then purified by prep-HPLC to give 9.3 mg (52%) of Example 178 as a white solid. ¹H-NMR (400 MHz, CD₃OD, ppm): δ 7.63 (s, 0.4H), 7.41 (s, 0.6H), 7.32 (s, 0.7H), 7.20 (dd, J=8.3, 1.4 Hz, 1.3H), 7.17-7.01 (m, 2H), 6.73-6.59 (m, 1H), 4.86-4.65 (m, 1H), 4.23 (d, J=10.3 Hz, 1H), 4.17-3.86 (m, 3H), 3.84-3.80 (m, 1H), 3.79 (d, J=3.3 Hz, 1H), 3.76 (d, J=3.5 Hz, 1H), 3.72 (dd, J=4.5, 2.1 Hz, 1H), 3.71-3.66 (m, 1H), 3.66-3.63 (m, 1H), 3.63-3.58 (m, 1H), 3.47 (d, J=4.7 Hz, 0.4H), 3.43 (d, J=4.7 Hz, 0.6H), 3.39 (dd, J=11.3, 5.8 Hz, 1H), 3.36-3.32 (m, 2H), 3.24 (dd, J=13.8, 7.4 Hz, 1H), 3.19-3.04 (m, 2H), 2.73 (t, J=7.2 Hz, 2H), 2.49-2.34 (m, 1H), 2.33-2.15 (m, 2H), 1.75-1.51 (m, 4H), 0.89-0.74 (m, 2H), 0.67-0.40 (m, 2H). LCMS (ES, m/z): 711.24 [M+H]⁺.

### Example 179

### (4-cyclopropyl-3,4-dihydroquinoxalin-1(2H)-yl)(3-(2,5-dichlorobenzyloxyl)oxetan-3-yl)methanone

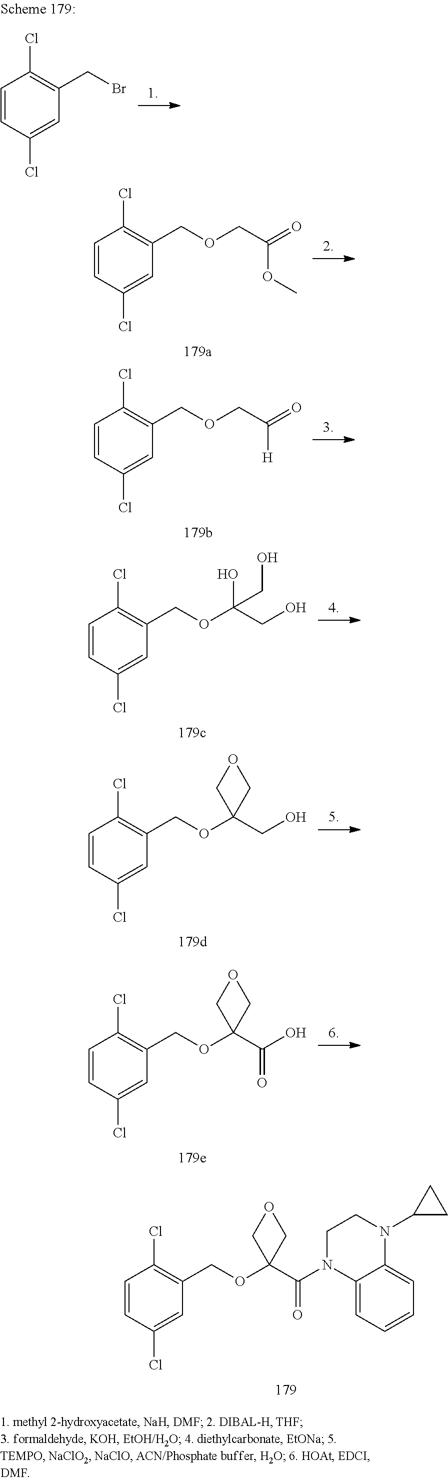

Intermediate 179a: methyl 2-(2,5-dichlorobenzyloxyl)acetate. To 2-(bromomethyl)-1,4-dichlorobenzene (5 g, 20.84 mmol, 1.00 equiv) in DMF (30 mL) was added methyl 2-hydroxyacetate (1.89 g, 20.98 mmol, 1.01 equiv) and the solution cooled to 0° C. To this was added in portions sodium hydride (1.0 g, 41.67 mmol, 2.00 equiv), and the resulting solution was allowed to warm to RT and then stirred overnight. The reaction was quenched by the addition of 50 mL of water, the resulting solution was extracted with 3×50 mL of ethyl acetate and the organic layers were combined and then washed with 1×50 mL of brine. The organic layer was dried over anhydrous sodium sulfate and concentrated under vacuum to afford 5.2 g (80%) of intermediate 179a as yellow oil.

Intermediate 179b: 2-(2,5-dichlorobenzyloxyl)acetaldehyde. To intermediate 179a (2 g, 8.03 mmol, 1.00 equiv) in THF (10 mL) at −60° C. was added drop-wise DIBAL-H (25% w/w in Hexane; 6.87 g, 12.10 mmol, 1.50 equiv) and the resulting solution was stirred for 3 h. The reaction was then quenched by the addition of water (20 mL), the pH value of the solution was adjusted to 5 with 1 M aqueous HCl and the resulting solution was extracted with 3×20 mL of ethyl acetate. The organic layers were combined, dried over anhydrous sodium sulfate and then concentrated under vacuum to afford 1.72 g (98%) of intermediate 179b as a yellow oil.

Intermediate 179c: 2-(2,5-dichlorobenzyloxy)-2-(hydroxymethyl)propane-1,3-diol. To intermediate 179b (1.72 g, 7.85 mmol, 1.00 equiv) in 1:1 ethanol/H₂O (10 mL) was added formaldehyde (40% in water; 5.91 g, 78.73 mmol, 10.00 equiv) followed by the drop-wise addition of a solution of potassium hydroxide (442 mg, 7.88 mmol, 1.00 equiv) in 1:1 ethanol/H₂O (5 mL) and the resulting solution was stirred for 3 h. The mixture was concentrated under vacuum, diluted with water (10 mL) and then extracted with 3×20 mL of ethyl acetate. The organic layers were combined, dried over anhydrous sodium sulfate, concentrated and then purified by silica gel chromatography (dichloromethane/methanol 50/1) to afford 700 mg (32%) of intermediate 179c as a white solid.

Intermediate 179d: (3-(2,5-dichlorobenzyloxyl)oxetan-3-yl)methanol. To intermediate 179c (340 mg, 1.21 mmol, 1.00 equiv) was added diethyl carbonate (215 mg, 1.82 mmol, 1.50 equiv) and sodium ethoxide (160 mg, 2.35 mmol, 0.20 equiv) and the resulting solution was stirred for 1 h at 140° C. and then for an additional 1 h at 190° C. The reaction mixture was cooled to RT, quenched by the addition of water (10 mL) and the resulting solution was extracted with 3×20 mL of ethyl acetate. The organic layers were combined, dried over anhydrous sodium sulfate, concentrated and then purified by preparative TLC (dichloromethane/methanol, 25/1) to afford 100 mg (31%) of intermediate 179d as yellow oil.

Intermediate 179e: 3-(2,5-dichlorobenzyloxy)oxetane-3-carboxylic acid. To intermediate 179d (150 mg, 0.57 mmol, 1.00 equiv) in ACN/phosphate buffer (7/3.5 mL) was added TEMPO (8.9 mg, 0.06 mmol, 0.10 equiv), NaClO₂ (129 mg, 1.43 mmol, 2.51 equiv), NaClO (11% in water; 19 mg, 0.03 mmol, 0.05 equiv) and the resulting solution was stirred for 20 h at 77° C. The pH value of the solution was adjusted to 5-6 with aqueous 1 M HCl and the resulting solution was extracted with 3×20 mL of ethyl acetate. The organic layers were combined, dried over anhydrous sodium sulfate and then concentrated under vacuum to afford 130 mg (82%) of intermediate 179e as a yellow oil.

Example 179: (4-cyclopropyl-3,4-dihydroquinoxalin-1(2H)-yl)(3-(2,5-dichlorobenzyloxyl)oxetan-3-yl)methanone. To intermediate 1e (130 mg, 0.47 mmol, 1.00 equiv) in DMF (3 mL) was added 1-cyclopropyl-1,2,3,4-tetrahydroquinoxaline (82 mg, 0.47 mmol, 1.00 equiv), HOAt (128 mg, 0.94 mmol, 2.00 equiv) and EDC-HCl (180 mg, 0.94 mmol, 2.00 equiv) and the resulting solution was stirred for 2 h. The reaction was quenched by the addition of water (10 mL) and then extracted with 3×20 mL of ethyl acetate. The organic layers were combined, washed with 1×10 mL of brine, dried over anhydrous sodium sulfate, concentrated and the crude product (100 mg) was purified by reverse-phase (C18) Prep-HPLC to afford 30 mg (15%) of Example 179 trifluoroacetic acid salt as an off-white solid. LCMS (ES, m/z): 433 [M+H]⁺, ¹H-NMR (300 MHz, CDCl3, ppm): 7.52-7.27 (m, 1H), 7.26-7.05 (m, 5H), 6.87-6.50 (m, 1H), 5.20-5.17 (m, 1H), 4.83-4.77 (m, 2H), 4.52 (s, 3H), 3.95-3.61 (m, 2H), 3.47-3.37 (m, 2H), 2.37 (s, 1H), 0.88-0.79 (m, 2H), 0.68-0.60 (m, 1H), 0.42 (s, 1H).

### Example 180

### (4-cyclopropyl-3,4-dihydroquinoxalin-1(2H)-yl)(1-((2,5-dichlorophenylthio)methyl)cyclopropyl)methanone

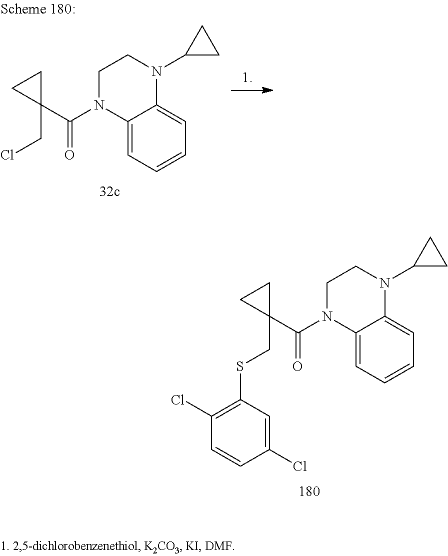 [SMILES: O=C(N1CCN(C2CC2)c2ccccc21)C1(CCl)CC1 ; O=C(N1CCN(C2CC2)c2ccccc21)C1(CSc2cc(Cl)ccc2Cl)CC1]

Example 180: (4-cyclopropyl-3,4-dihydroquinoxalin-1(2H)-yl)(1-((2,5-dichloro-phenylthio)methyl)cyclopropyl)methanone. To intermediate 32c (30 mg, 0.10 mmol, 1.00 equiv) in DMF (2.0 mL) was added 2,5-dichlorobenzenethiol (27.7 mg, 0.15 mmol, 1.00 equiv), potassium carbonate (27.6 mg, 0.20 mmol, 2.00 equiv), and KI (1.7 mg, 0.01 mmol, 0.10 equiv) and the resulting solution was stirred overnight. The reaction was quenched by the addition of water (5 mL) and the resulting solution extracted with 3×5.0 mL of ethyl acetate, the organic layers were combined, dried over anhydrous sodium sulfate and then concentrated under vacuum. The crude product was purified by reverse-phase (C18) Prep-HPLC to afford 7.8 mg (17%) of Example 180 as a light yellow solid. LCMS (ES, m/z): 433[M+H]⁺; ¹H-NMR (300 Hz, CD3OD, ppm): 7.30-7.34 (m, 2H), 7.05-7.16 (m, 3H), 6.74-6.80 (m, 1H), 6.69-6.74 (m, 1H), 3.83-3.87 (m, 2H), 3.43-3.46 (m, 2H), 2.70 (s, 2H), 2.20-2.27 (m, 1H), 1.43-1.45 (m, 2H), 0.70-0.90 (m, 2H), 0.67-0.69 (m, 2H), 0.20-0.30 (m, 2H).

### Example 181

### (R)-(3-(4-(5-guanidinylpentyl)-2,5-dichlorobenzyl)thiazolidin-4-yl)(4-cyclopropyl-3,4-dihydroquinoxalin-1(2H)-yl)methanone

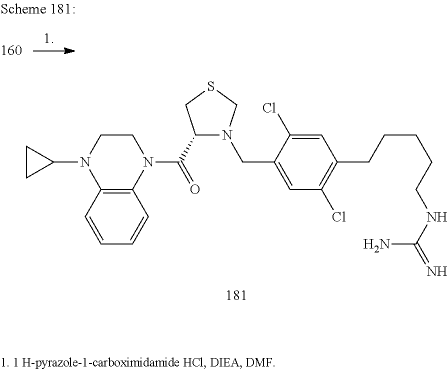 [SMILES: N=C(N)NCCCCCc1cc(Cl)c(CN2CSC[C@H]2C(=O)N2CCN(C3CC3)c3ccccc32)cc1Cl]

Example 181: (R)-(3-(4-(5-guanidinylpentyl)-2,5-dichlorobenzyl)thiazolidin-4-yl)(4-cyclopropyl-3,4-dihydroquinoxalin-1(2H)-yl)methanone. To intermediate 160 (70 mg, 0.13 mmol, 1.00 equiv) in DMF (3 mL) at 5° C. was added 1H-pyrazole-1-carboximidamide hydrochloride (38.7 mg, 0.26 mmol, 2.00 equiv) and DIEA (101.8 mg, 0.79 mmol, 6.00 equiv) and the reaction was allowed to warm to RT and then stirred overnight. The reaction was diluted with 10 mL of H₂O, extracted with 2×15 mL of ethyl acetate and then the organic layers were combined, washed with 2×30 mL of sodium chloride and dried over anhydrous sodium sulfate. The solution was concentrated and the residue was purified via reverse-phase (C18) Prep-HPLC to afford 32.7 mg (43%) of Example 181 tri-trifluoroacetate as a white solid. LCMS (ES, m/z): 575 [M+H]⁺. ¹H-NMR (400 MHz, CD₃OD, ppm): 7.36-7.20 (m, 2H), 7.17 (m, 1H), 7.12-7.07 (m, 2H), 6.68-6.65 (m, 1H), 4.93-4.56 (s, 1H), 4.11-4.09 (m, 2H), 3.88-3.71 (m, 3H), 3.50-3.42 (m, 1H), 3.40-3.46 (m, 3H), 3.33-3.32 (m, 1H), 3.24-3.21 (m, 2H), 3.19-3.12 (m, 1H), 2.76-2.68 (m, 2H), 2.44 (m, 1H), 1.71-1.62 (m, 4H), 1.50-1.42 (m, 2H), 0.84-0.82 (m, 2H), 0.60-0.50 (m, 2H).

### Example 182

### 4-(2,5-dichloro-4-(((R)-4-(4-cyclopropyl-1,2,3,4-tetrahydroquinoxaline-1-carbonyl)thiazolidin-3-yl)methyl)benzyloxy)-N-((2S,3R,4R,5R)-2,3,4,5,6-pentahydroxyhexyl)benzamide

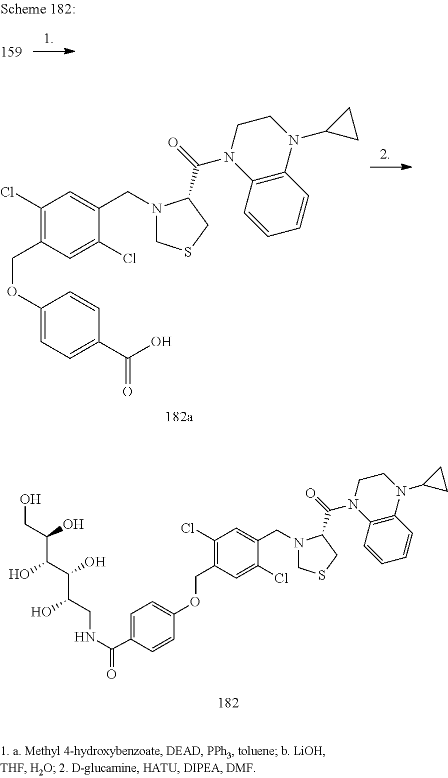 [SMILES: O=C(O)c1ccc(OCc2cc(Cl)c(CN3CSC[C@H]3C(=O)N3CCN(C4CC4)c4ccccc43)cc2Cl)cc1 ; O=C(NC[C@H](O)[C@@H](O)[C@H](O)[C@H](O)CO)c1ccc(OCc2cc(Cl)c(CN3CSC[C@H]3C(=O)N3CCN(C4CC4)c4ccccc43)cc2Cl)cc1]

Intermediate 182a: (R)-4-(2,5-dichloro-4-((4-(4-cyclopropyl-1,2,3,4-tetrahydroquinoxaline-1-carbonyl)thiazolidin-3-yl)methyl)benzyloxy)benzoic acid. Intermediate 182a was prepared using the procedures described for the preparation of example 161 except that methyl 4-hydroxybenzoate was used in place of methyl 2-mercapto-1-methyl-1H-imidazole-5-carboxylate.

Example 182: 4-(2,5-dichloro-4-(((R)-4-(4-cyclopropyl-1,2,3,4-tetrahydroquinoxaline-1-carbonyl)thiazolidin-3-yl)methyl)benzyloxy)-N-((2S,3R,4R,5R)-2,3,4,5,6-pentahydroxyhexyl)benzamide. Example 182 was prepared using the procedures described for the preparation of example 175 to provide the title compound bis TFA salt as a white solid. (ES, m/z): 761 [M+H]⁺, (400 MHz, CD₃OD, ppm): 7.85-7.87 (m, 2H), 7.65-7.67 (m, 1H), 7.59 (s, 1H), 7.12-7.34 (m, 5H), 6.69 (s, 1H), 5.23 (s, 2H), 3.63-4.31 (m, 18H), 3.19-3.33 (m, 2H), 2.45 (s, 1H), 0.83-0.84 (m, 2H), 0.56 (m, 2H).

### Example 183

### (1-(2-chloro-5-cyclopropylbenzylamino)cyclopropyl)(4-cyclopropyl-3,4-dihydroquinoxalin-1(2H)-yl)methanone

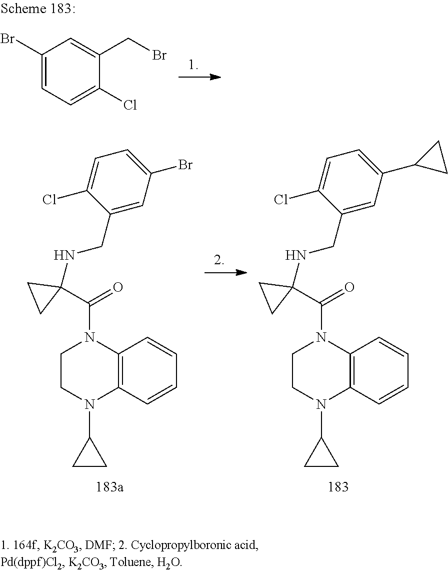

Intermediate 183a: (1-(5-bromo-2-chlorobenzylamino)cyclopropyl)(4-cyclopropyl-3,4-dihydroquinoxalin-1(2H)-yl)methanone. To a solution of intermediate 164f (100 mg, 0.39 mmol, 1.00 equiv) in DMF (3 mL) was added 4-bromo-2-(bromomethyl)-1-chlorobenzene (128.4 mg, 0.45 mmol, 1.16 equiv) and potassium carbonate (107.4 mg, 0.78 mmol, 2.00 equiv) and the reaction was stirred overnight. The mixture was diluted with 10 mL of H₂O, extracted with 25 mL of ethyl acetate, and the organic layer was washed with brine (2×25 mL), dried over anhydrous sodium sulfate and concentrated to afford 200 mg crude 183a as brown oil.

Example 183 (1-(2-chloro-5-cyclopropylbenzylamino)cyclopropyl)(4-cyclopropyl-3,4-dihydroquinoxalin-1(2H)-yl)methanone. To 183a (160 mg, 0.35 mmol, 1.00 equiv) in toluene/H₂O (2/0.2 mL) was added cyclopropylboronic acid (90 mg, 1.05 mmol, 3.00 equiv), Pd(dppf)Cl₂ (25 mg, 0.03 mmol, 0.10 equiv) and potassium carbonate (145 mg, 1.05 mmol, 3.00 equiv) and the reaction was stirred at 80° C. overnight. The mixture was then concentrated under vacuum and the crude product (200 mg) purified by reverse-phase (C18) prep-HPLC to afford 31.9 mg (22%) of Example 183 as a brown solid. (ES, m/z): 422 [M+H]⁺ (300 MHz, CD₃OD, ppm): 7.28 (d, J=6.6 Hz, 1H), 7.26-7.12 (m, 3H), 6.94 (d, J=2.4 Hz, 1H), 6.72-6.62 (m, 1H), 6.62 (s, 1H), 3.86-3.79 (m, 4H), 3.40-3.36 (m, 2H), 2.37 (m, 1H), 1.79 (m, 1H), 1.37-1.35 (m, 2H), 1.07-1.05 (m, 2H), 0.94-0.91 (m, 2H), 0.76-0.74 (m, 2H), 0.60-0.56 (m, 2H), 0.43-0.41 (m, 2H).

### Examples 184-291

Table 1 illustrates the method of preparation for examples 184-291, which were prepared using commercial or known starting materials according to the general methods described in examples 1-98 and 159-183 and methods generally known to those skilled in the art.

General synthetic method A: addition of an amine nucleophile intermediate, for example 160 or the like, with a reactive di or trihaloaromatic or heteroaromatic ring, such as 2,4,6-trichloro-1,3,5-triazine, followed by sequential reaction with one or more amine nucleophiles to provide the example compound. The synthesis of Example 173 is a typical procedure.

General synthetic method B: hydroboration of an alkene followed by Suzuki coupling to a halide or triflate intermediate, for example intermediate 174d or the like. This method allows incorporation of one or more reactive functional groups and permits further functionality to be appended. The synthesis of Example 174 is a typical procedure.

General synthetic method C: reductive amination of a dialdehyde to provide an alcohol intermediate such as 159. Mitsunobu alkylation allows additional functionality to be appended to the free alcohol. The synthesis of Example 182 is a typical procedure.

General synthetic method D: reductive amination with a hydroxyaldehyde to provide a phenol intermediate such as example 162. Mitsunobu alkylation allows additional functionality to be appended. The synthesis of Example 176 is a typical procedure.

General synthetic method E: the synthesis of alkyamine, alkylcarboxylate, and alcohol intermediates such as 160, 178g and 171j by Sonogashira coupling to an arylhalide or triflate followed by reduction of the alkyne is demonstrated in several examples. Typical functional group transformations allow functionality to be appended to these intermediates through ureas, amides amines, guanidines and sulfonamides. The synthesis of examples 171, 172, 177, 178, and 181, are typical procedures.

TABLE 1

| Example | Structure | Method | Mass | Mass |
|---|---|---|---|---|
| 184 | 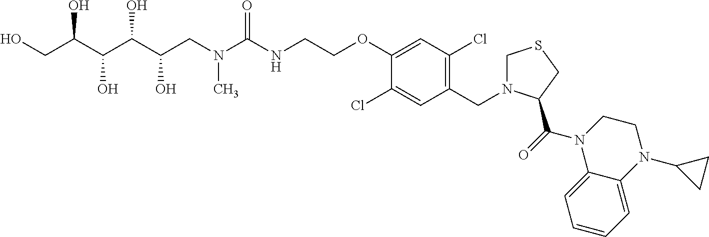 [SMILES: CN(C[C@H](O)[C@@H](O)[C@H](O)[C@H](O)CO)C(=O)NCCOc1cc(Cl)c(CN2CSC[C@H]2C(=O)N2CCN(C3CC3)c3ccccc32)cc1Cl] | D | 727.22 | 728.32 [M +H]+ |
| 185 | 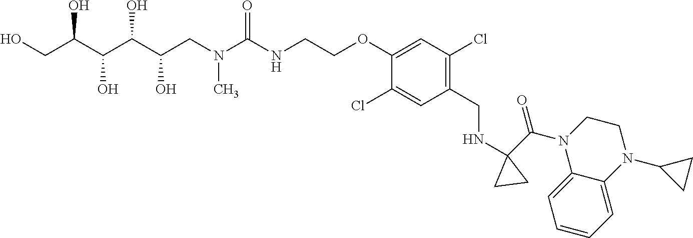 [SMILES: CN(C[C@H](O)[C@@H](O)[C@H](O)[C@H](O)CO)C(=O)NCCOc1cc(Cl)c(CNC2(C(=O)N3CCN(C4CC4)c4ccccc43)CC2)cc1Cl] | D | 695.25 | 696.27 [M +H]+ |
| 186 | 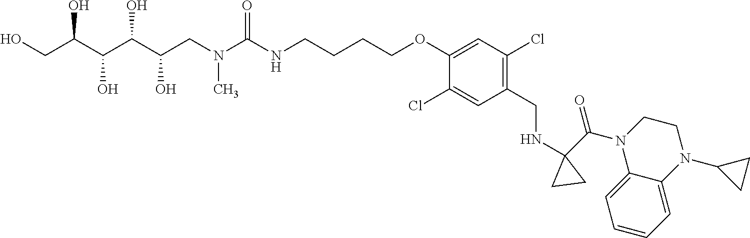 [SMILES: CN(C[C@H](O)[C@@H](O)[C@H](O)[C@H](O)CO)C(=O)NCCCCOc1cc(Cl)c(CNC2(C(=O)N3CCN(C4CC4)c4ccccc43)CC2)cc1Cl] | D | 723.28 | 724.20 [M +H]+ |
| 187 | 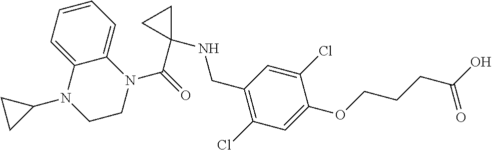 [SMILES: O=C(O)CCCOc1cc(Cl)c(CNC2(C(=O)N3CCN(C4CC4)c4ccccc43)CC2)cc1Cl] | D | 517.15 | 517.99 [M +H]+ |
| 188 | 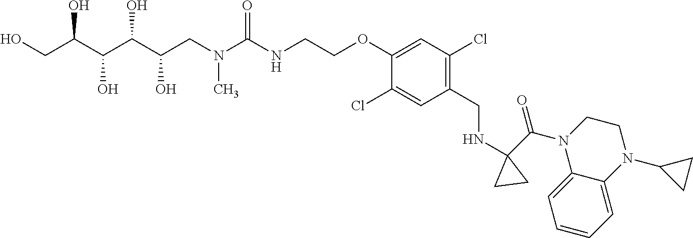 [SMILES: CN(C[C@H](O)[C@@H](O)[C@H](O)[C@H](O)CO)C(=O)NCCOc1cc(Cl)c(CNC2(C(=O)N3CCN(C4CC4)c4ccccc43)CC2)cc1Cl] | D | 694.25 | 695.22 [M +H]+ |
| 189 | 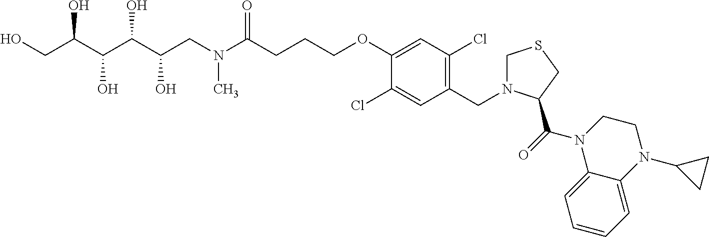 [SMILES: CN(C[C@H](O)[C@@H](O)[C@H](O)[C@H](O)CO)C(=O)CCCOc1cc(Cl)c(CN2CSC[C@H]2C(=O)N2CCN(C3CC3)c3ccccc32)cc1Cl] | D | 726.23 | 727.30 [M +H]+ |
| 190 | 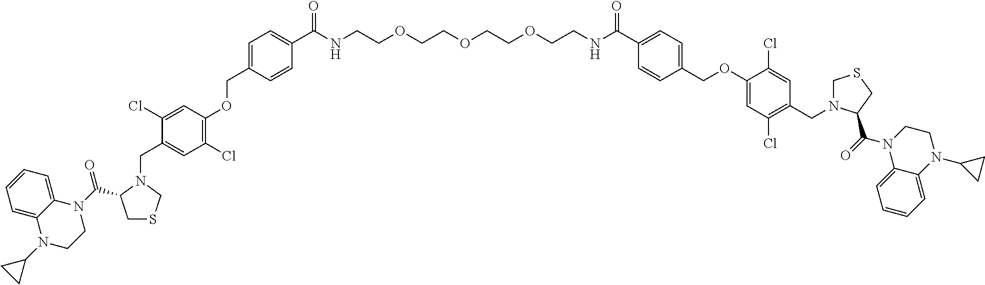 [SMILES: O=C(NCCOCCOCCOCCNC(=O)c1ccc(COc2cc(Cl)c(CN3CSC[C@H]3C(=O)N3CCN(C4CC4)c4ccccc43)cc2Cl)cc1)c1ccc(COc2cc(Cl)c(CN3CSC[C@@H]3C(=O)N3CCN(C4CC4)c4ccccc43)cc2Cl)cc1] | D | 1350.38 | 1351.38 [M +H]+ |
| 191 | 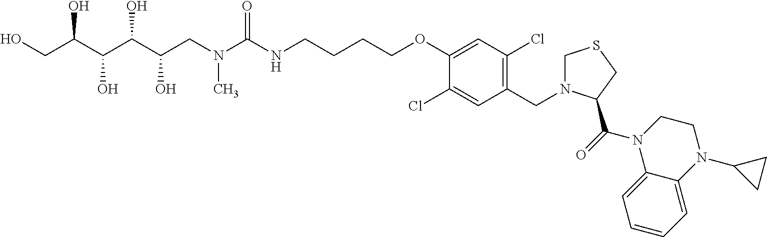 [SMILES: CN(C[C@H](O)[C@@H](O)[C@H](O)[C@H](O)CO)C(=O)NCCCCOc1cc(Cl)c(CN2CSC[C@H]2C(=O)N2CCN(C3CC3)c3ccccc32)cc1Cl] | D | 755.25 | 756.30 [M +H]+ |
| 192 | 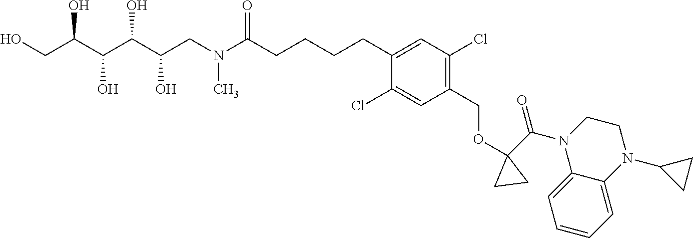 [SMILES: CN(C[C@H](O)[C@@H](O)[C@H](O)[C@H](O)CO)C(=O)CCCCc1cc(Cl)c(COC2(C(=O)N3CCN(C4CC4)c4ccccc43)CC2)cc1Cl] | E | 693.26 | 694.20 [M +H]+ |
| 193 | 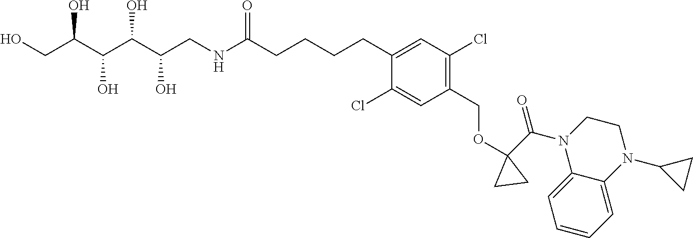 [SMILES: O=C(CCCCc1cc(Cl)c(COC2(C(=O)N3CCN(C4CC4)c4ccccc43)CC2)cc1Cl)NC[C@H](O)[C@@H](O)[C@H](O)[C@H](O)CO] | E | 679.24 | 680.29 [M +H]+ |
| 194 | 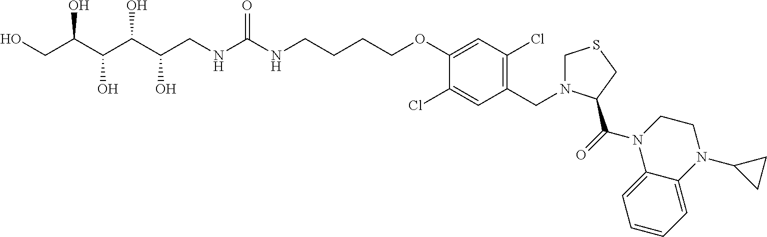 [SMILES: O=C(NCCCCOc1cc(Cl)c(CN2CSC[C@H]2C(=O)N2CCN(C3CC3)c3ccccc32)cc1Cl)NC[C@H](O)[C@@H](O)[C@H](O)[C@H](O)CO] | D | 741.24 | 742.17 [M +H]+ |
| 195 | 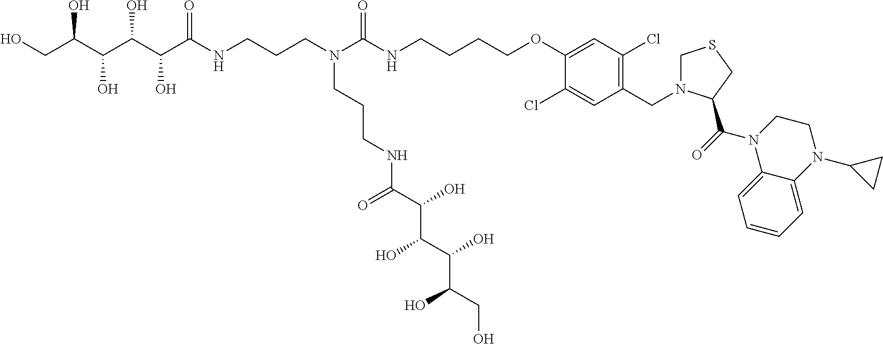 [SMILES: O=C(NCCCN(CCCNC(=O)[C@H](O)[C@@H](O)[C@H](O)[C@H](O)CO)C(=O)NCCCCOc1cc(Cl)c(CN2CSC[C@H]2C(=O)N2CCN(C3CC3)c3ccccc32)cc1Cl)[C@H](O)[C@@H](O)[C@H](O)[C@H](O)CO] | D | 1047.38 | 1048.34 [M +H]+ |
| 196 | 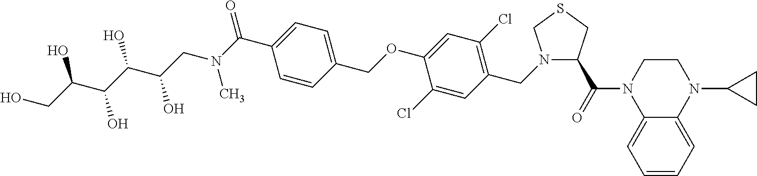 [SMILES: CN(C[C@H](O)[C@@H](O)[C@H](O)[C@H](O)CO)C(=O)c1ccc(COc2cc(Cl)c(CN3CSC[C@H]3C(=O)N3CCN(C4CC4)c4ccccc43)cc2Cl)cc1] | D | 774.23 | 775.26 [M +H]+ |
| 197 | 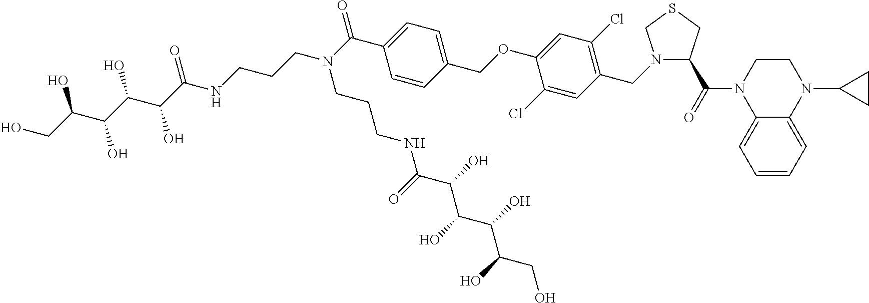 [SMILES: O=C(NCCCN(CCCNC(=O)[C@H](O)[C@@H](O)[C@H](O)[C@H](O)CO)C(=O)c1ccc(COc2cc(Cl)c(CN3CSC[C@H]3C(=O)N3CCN(C4CC4)c4ccccc43)cc2Cl)cc1)[C@H](O)[C@@H](O)[C@H](O)[C@H](O)CO] | D | 1066.35 | 1067.46 [M +H]+ |
| 198 | 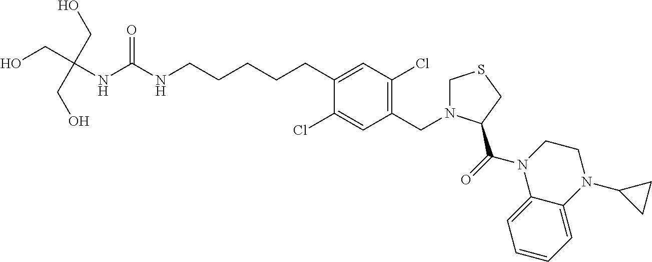 [SMILES: O=C(NCCCCCc1cc(Cl)c(CN2CSC[C@H]2C(=O)N2CCN(C3CC3)c3ccccc32)cc1Cl)NC(CO)(CO)CO] | E | 679.24 | 680.33 [M +H]+ |
| 199 | 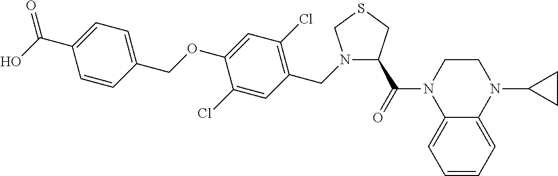 [SMILES: O=C(O)c1ccc(COc2cc(Cl)c(CN3CSC[C@H]3C(=O)N3CCN(C4CC4)c4ccccc43)cc2Cl)cc1] | D | 597.13 | 598.09 [M +H]+ |
| 200 | 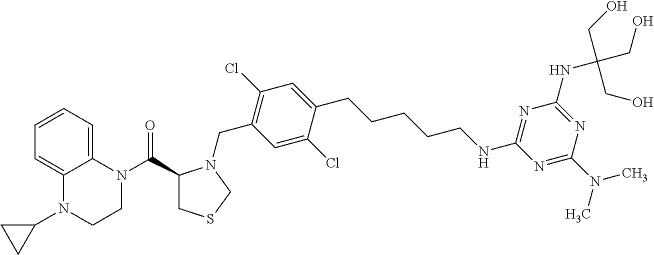 [SMILES: CN(C)c1nc(NCCCCCc2cc(Cl)c(CN3CSC[C@H]3C(=O)N3CCN(C4CC4)c4ccccc43)cc2Cl)nc(NC(CO)(CO)CO)n1] | A | 773.30 | 774.19 [M +H]+ |
| 201 | 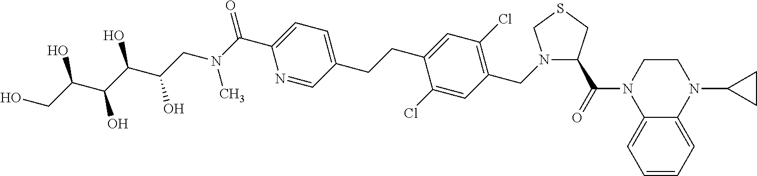 [SMILES: CN(C[C@H](O)[C@H](O)[C@@H](O)[C@H](O)CO)C(=O)c1ccc(CCc2cc(Cl)c(CN3CSC[C@H]3C(=O)N3CCN(C4CC4)c4ccccc43)cc2Cl)cn1] | E | 773.24 | 774.22 [M +H]+ |
| 202 | 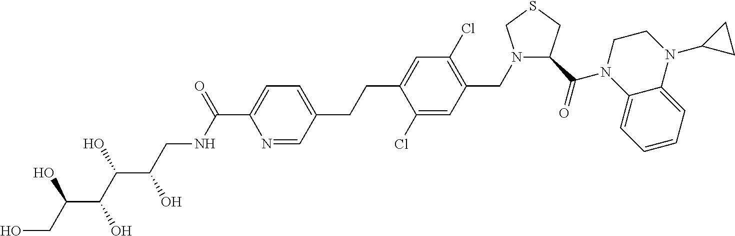 [SMILES: O=C(NC[C@H](O)[C@@H](O)[C@H](O)[C@H](O)CO)c1ccc(CCc2cc(Cl)c(CN3CSC[C@H]3C(=O)N3CCN(C4CC4)c4ccccc43)cc2Cl)cn1] | E | 759.23 | 760.28 [M +H]+ |
| 203 | 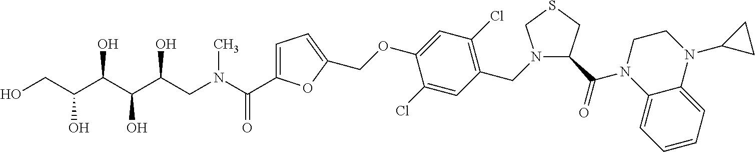 [SMILES: CN(C[C@H](O)[C@@H](O)[C@H](O)[C@H](O)CO)C(=O)c1ccc(COc2cc(Cl)c(CN3CSC[C@H]3C(=O)N3CCN(C4CC4)c4ccccc43)cc2Cl)o1] | D | 764.21 | 765.19 [M +H]+ |
| 204 | 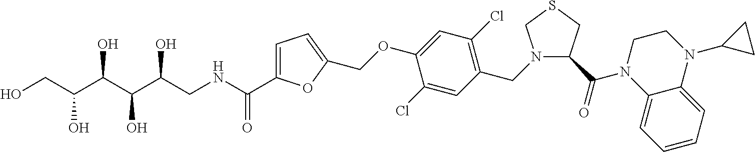 [SMILES: O=C(NC[C@H](O)[C@@H](O)[C@H](O)[C@H](O)CO)c1ccc(COc2cc(Cl)c(CN3CSC[C@H]3C(=O)N3CCN(C4CC4)c4ccccc43)cc2Cl)o1] | D | 750.19 | 751.28 [M +H]+ |
| 205 | 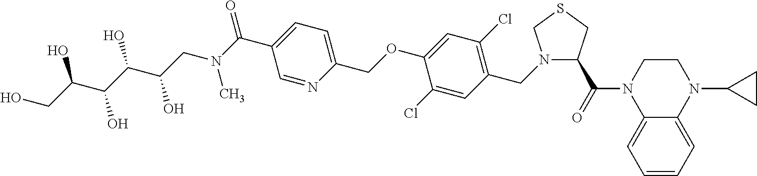 [SMILES: CN(C[C@H](O)[C@@H](O)[C@H](O)[C@H](O)CO)C(=O)c1ccc(COc2cc(Cl)c(CN3CSC[C@H]3C(=O)N3CCN(C4CC4)c4ccccc43)cc2Cl)nc1] | D | 775.22 | 776.29 [M +H]+ |
| 206 | 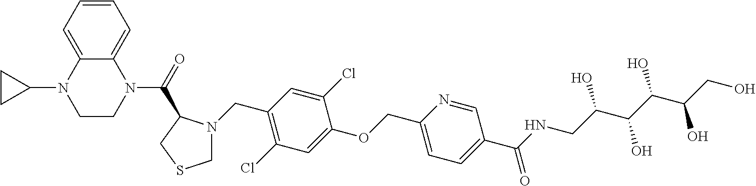 [SMILES: O=C(NC[C@H](O)[C@@H](O)[C@H](O)[C@H](O)CO)c1ccc(COc2cc(Cl)c(CN3CSC[C@H]3C(=O)N3CCN(C4CC4)c4ccccc43)cc2Cl)nc1] | D | 761.21 | 762.27 [M +H]+ |
| 207 | 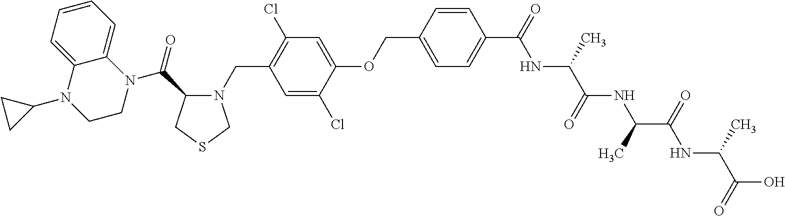 [SMILES: C[C@@H](NC(=O)[C@@H](C)NC(=O)[C@@H](C)NC(=O)c1ccc(COc2cc(Cl)c(CN3CSC[C@H]3C(=O)N3CCN(C4CC4)c4ccccc43)cc2Cl)cc1)C(=O)O] | D | 810.24 | 811.56 [M +H]+ |
| 208 | 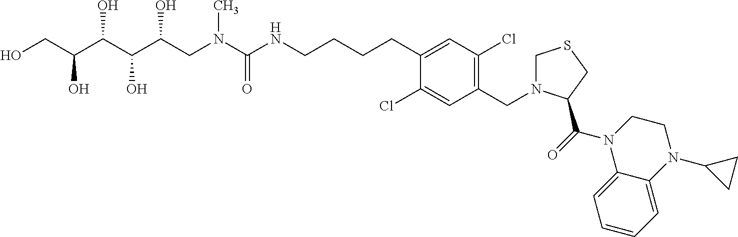 [SMILES: CN(C[C@@H](O)[C@H](O)[C@@H](O)[C@@H](O)CO)C(=O)NCCCCc1cc(Cl)c(CN2CSC[C@H]2C(=O)N2CCN(C3CC3)c3ccccc32)cc1Cl] | E | 739.26 | 740.51 [M +H]+ |
| 209 | 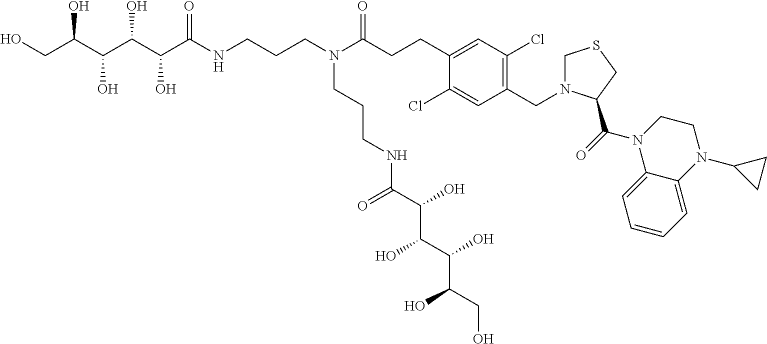 [SMILES: O=C(NCCCN(CCCNC(=O)[C@H](O)[C@@H](O)[C@H](O)[C@H](O)CO)C(=O)CCc1cc(Cl)c(CN2CSC[C@H]2C(=O)N2CCN(C3CC3)c3ccccc32)cc1Cl)[C@H](O)[C@@H](O)[C@H](O)[C@H](O)CO] | Example 48 | 988.34 | 989.27 [M +H]+ |
| 210 | 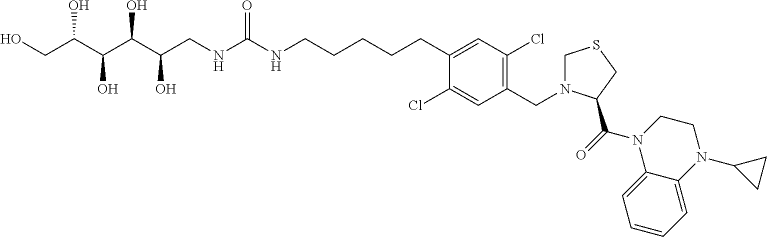 [SMILES: O=C(NCCCCCc1cc(Cl)c(CN2CSC[C@H]2C(=O)N2CCN(C3CC3)c3ccccc32)cc1Cl)NC[C@@H](O)[C@H](O)[C@@H](O)[C@@H](O)CO] | E | 739.26 | 740.15 [M +H]+ |
| 211 | 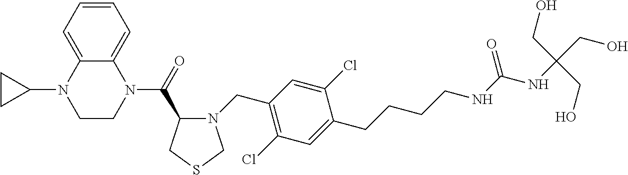 [SMILES: O=C(NCCCCc1cc(Cl)c(CN2CSC[C@H]2C(=O)N2CCN(C3CC3)c3ccccc32)cc1Cl)NC(CO)(CO)CO] | E | 665.22 | 666.21 [M +H]+ |
| 212 | 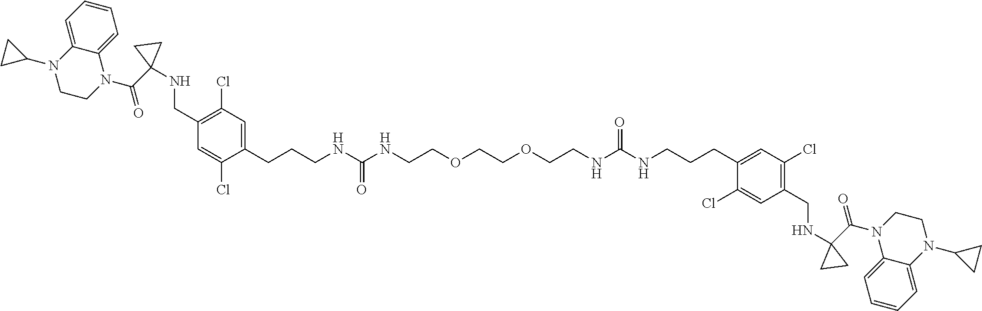 [SMILES: O=C(NCCCc1cc(Cl)c(CNC2(C(=O)N3CCN(C4CC4)c4ccccc43)CC2)cc1Cl)NCCOCCOCCNC(=O)NCCCc1cc(Cl)c(CNC2(C(=O)N3CCN(C4CC4)c4ccccc43)CC2)cc1Cl] | Example 93 | 1144.44 | 1147.29 [M +H]+ |
| 213 | 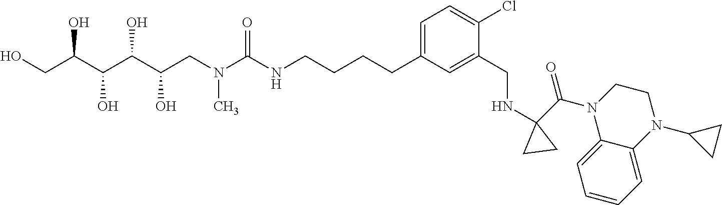 [SMILES: CN(C[C@H](O)[C@@H](O)[C@H](O)[C@H](O)CO)C(=O)NCCCCc1ccc(Cl)c(CNC2(C(=O)N3CCN(C4CC4)c4ccccc43)CC2)c1] | E | 673.32 | 674.31 [M +H]+ |
| 214 | 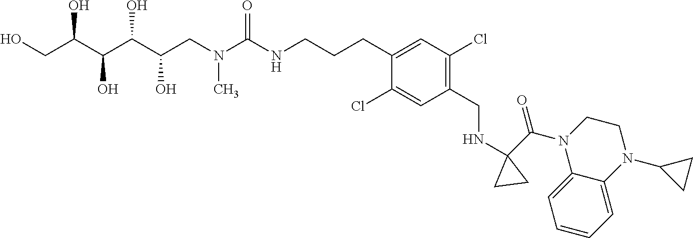 [SMILES: CN(C[C@H](O)[C@@H](O)[C@@H](O)[C@H](O)CO)C(=O)NCCCc1cc(Cl)c(CNC2(C(=O)N3CCN(C4CC4)c4ccccc43)CC2)cc1Cl] | E | 693.27 | 694.29 [M +H]+ |
| 215 | 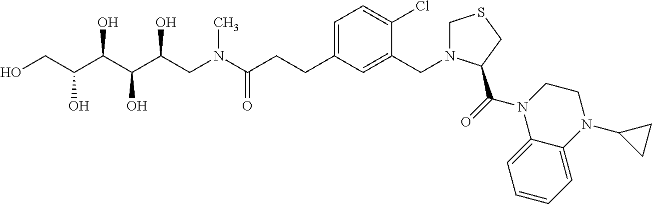 [SMILES: CN(C[C@H](O)[C@@H](O)[C@H](O)[C@H](O)CO)C(=O)CCc1ccc(Cl)c(CN2CSC[C@H]2C(=O)N2CCN(C3CC3)c3ccccc32)c1] | E | 662.25 | 663.17 [M +H]+ |
| 216 | 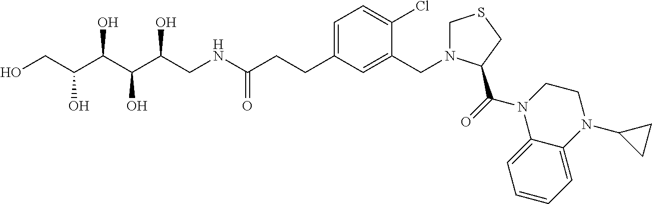 [SMILES: O=C(CCc1ccc(Cl)c(CN2CSC[C@H]2C(=O)N2CCN(C3CC3)c3ccccc32)c1)NC[C@H](O)[C@@H](O)[C@H](O)[C@H](O)CO] | Example 60 | 648.24 | 649.20 [M +H]+ |
| 217 | 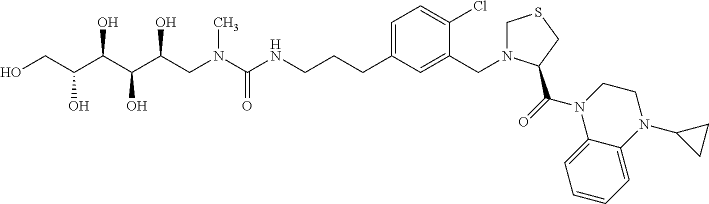 [SMILES: CN(C[C@H](O)[C@@H](O)[C@H](O)[C@H](O)CO)C(=O)NCCCc1ccc(Cl)c(CN2CSC[C@H]2C(=O)N2CCN(C3CC3)c3ccccc32)c1] | Example 69 | 691.28 | 692.25 [M +H]+ |
| 218 | 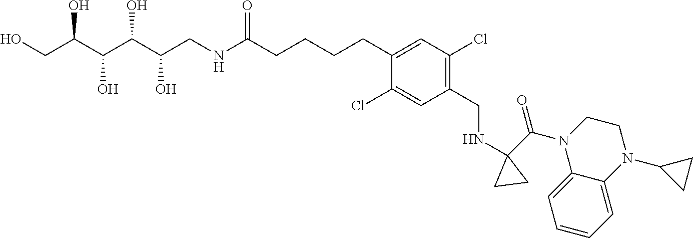 [SMILES: O=C(CCCCc1cc(Cl)c(CNC2(C(=O)N3CCN(C4CC4)c4ccccc43)CC2)cc1Cl)NC[C@H](O)[C@@H](O)[C@H](O)[C@H](O)CO] | E | 678.26 | 679.31 [M +H]+ |
| 219 | 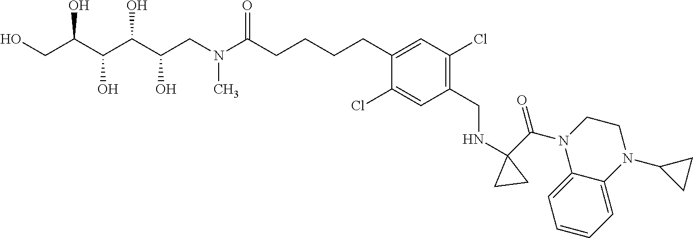 [SMILES: CN(C[C@H](O)[C@@H](O)[C@H](O)[C@H](O)CO)C(=O)CCCCc1cc(Cl)c(CNC2(C(=O)N3CCN(C4CC4)c4ccccc43)CC2)cc1Cl] | E | 692.27 | 693.30 [M +H]+ |
| 220 | 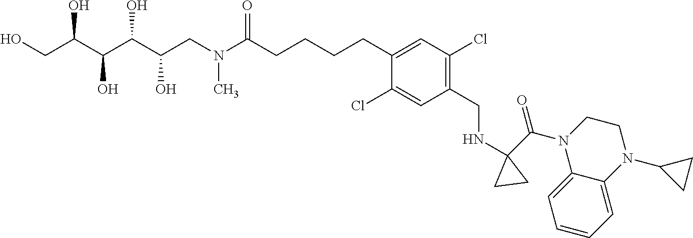 [SMILES: CN(C[C@H](O)[C@@H](O)[C@@H](O)[C@H](O)CO)C(=O)CCCCc1cc(Cl)c(CNC2(C(=O)N3CCN(C4CC4)c4ccccc43)CC2)cc1Cl] | E | 692.27 | 693.32 [M +H]+ |
| 221 | 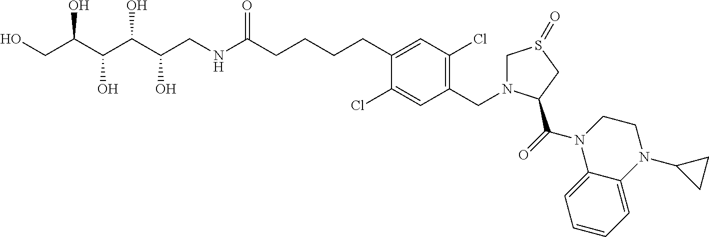 [SMILES: O=C(CCCCc1cc(Cl)c(CN2CS(=O)C[C@H]2C(=O)N2CCN(C3CC3)c3ccccc32)cc1Cl)NC[C@H](O)[C@@H](O)[C@H](O)[C@H](O)CO] | Isolated as a side product from the synthesis of Example 178 | 726.23 | 727.18 [M +H]+ |
| 222 | 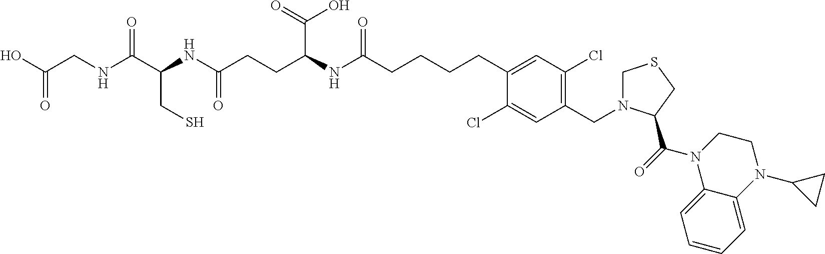 [SMILES: O=C(O)CNC(=O)[C@H](CS)NC(=O)CC[C@H](NC(=O)CCCCc1cc(Cl)c(CN2CSC[C@H]2C(=O)N2CCN(C3CC3)c3ccccc32)cc1Cl)C(=O)O] | E | 836.22 | 837.34 [M +H]+ |
| 223 | 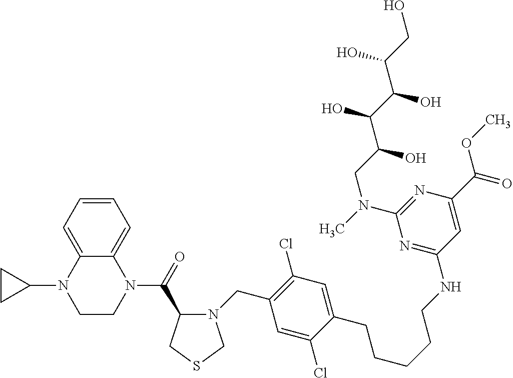 [SMILES: COC(=O)c1cc(NCCCCCc2cc(Cl)c(CN3CSC[C@H]3C(=O)N3CCN(C4CC4)c4ccccc43)cc2Cl)nc(N(C)C[C@H](O)[C@@H](O)[C@H](O)[C@H](O)CO)n1] | A | 861.31 | 862.33 [M +H]+ |
| 224 | 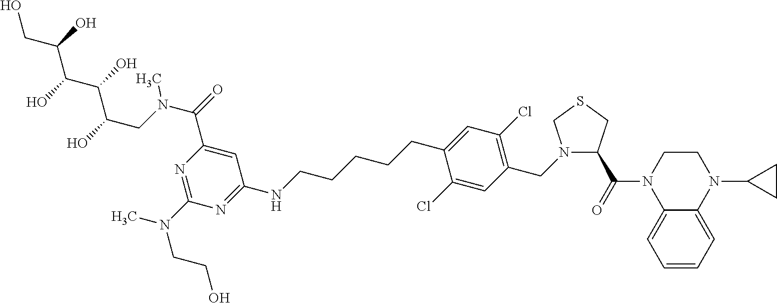 [SMILES: CN(C[C@H](O)[C@@H](O)[C@H](O)[C@H](O)CO)C(=O)c1cc(NCCCCCc2cc(Cl)c(CN3CSC[C@H]3C(=O)N3CCN(C4CC4)c4ccccc43)cc2Cl)nc(N(C)CCO)n1] | A | 904.35 | 905.29 [M +H]+ |
| 225 | 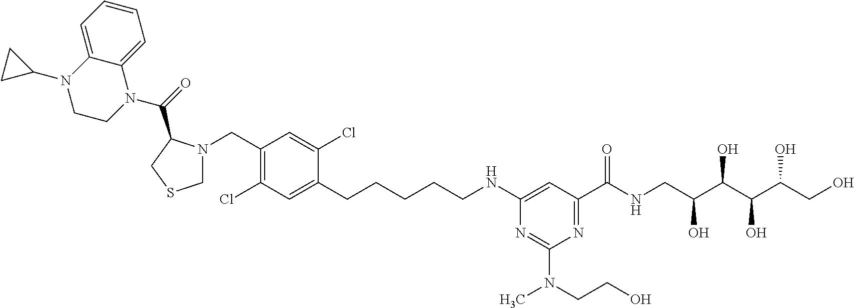 [SMILES: CN(CCO)c1nc(NCCCCCc2cc(Cl)c(CN3CSC[C@H]3C(=O)N3CCN(C4CC4)c4ccccc43)cc2Cl)cc(C(=O)NC[C@H](O)[C@@H](O)[C@H](O)[C@H](O)CO)n1] | A | 890.33 | 891.25 [M +H]+ |
| 226 | 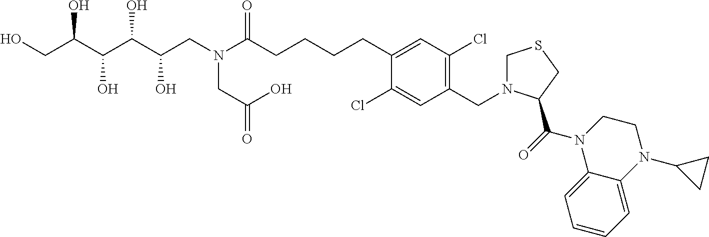 [SMILES: O=C(O)CN(C[C@H](O)[C@@H](O)[C@H](O)[C@H](O)CO)C(=O)CCCCc1cc(Cl)c(CN2CSC[C@H]2C(=O)N2CCN(C3CC3)c3ccccc32)cc1Cl] | E | 768.24 | 769.25 [M +H]+ |
| 227 | 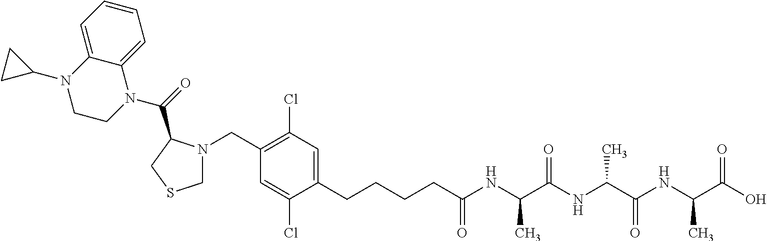 [SMILES: C[C@@H](NC(=O)[C@@H](C)NC(=O)[C@@H](C)NC(=O)CCCCc1cc(Cl)c(CN2CSC[C@H]2C(=O)N2CCN(C3CC3)c3ccccc32)cc1Cl)C(=O)O] | E | 760.26 | 761.31 [M +H]+ |
| 228 | 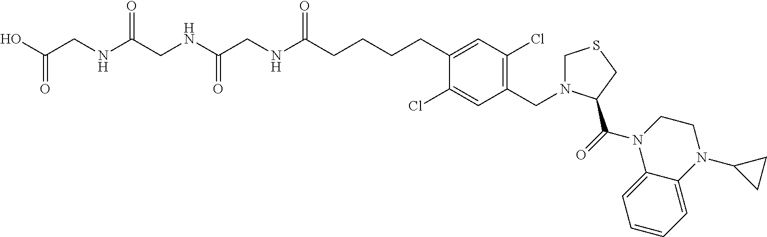 [SMILES: O=C(O)CNC(=O)CNC(=O)CNC(=O)CCCCc1cc(Cl)c(CN2CSC[C@H]2C(=O)N2CCN(C3CC3)c3ccccc32)cc1Cl] | E | 718.21 | 719.33 [M +H]+ |
| 229 | 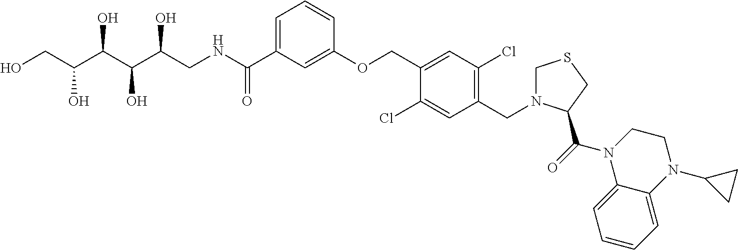 [SMILES: O=C(NC[C@H](O)[C@@H](O)[C@H](O)[C@H](O)CO)c1cccc(OCc2cc(Cl)c(CN3CSC[C@H]3C(=O)N3CCN(C4CC4)c4ccccc43)cc2Cl)c1] | D | 760.21 | 761.15 [M +H]+ |
| 230 | 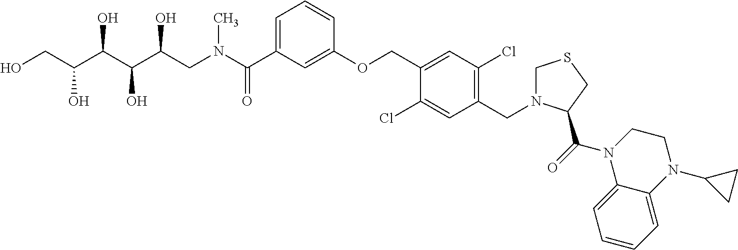 [SMILES: CN(C[C@H](O)[C@@H](O)[C@H](O)[C@H](O)CO)C(=O)c1cccc(OCc2cc(Cl)c(CN3CSC[C@H]3C(=O)N3CCN(C4CC4)c4ccccc43)cc2Cl)c1] | D | 774.23 | 775.22 [M +H]+ |
| 231 | 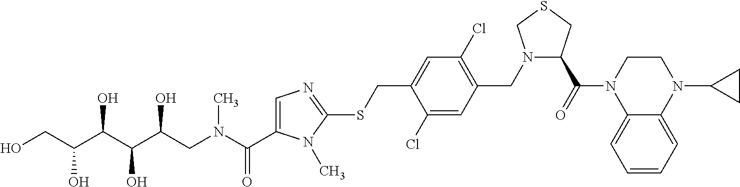 [SMILES: CN(C[C@H](O)[C@@H](O)[C@H](O)[C@H](O)CO)C(=O)c1cnc(SCc2cc(Cl)c(CN3CSC[C@H]3C(=O)N3CCN(C4CC4)c4ccccc43)cc2Cl)n1C] | C | 794.21 | 795.23 [M +H]+ |
| 232 | 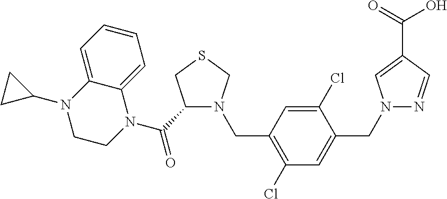 [SMILES: O=C(O)c1cnn(Cc2cc(Cl)c(CN3CSC[C@H]3C(=O)N3CCN(C4CC4)c4ccccc43)cc2Cl)c1] | C | 571.12 | 572.29 [M +H]+ |
| 233 | 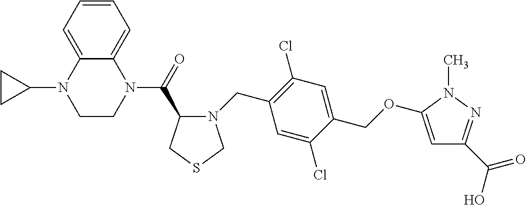 [SMILES: Cn1nc(C(=O)O)cc1OCc1cc(Cl)c(CN2CSC[C@H]2C(=O)N2CCN(C3CC3)c3ccccc32)cc1Cl] | C | 601.13 | 602.08 [M +H]+ |
| 234 | 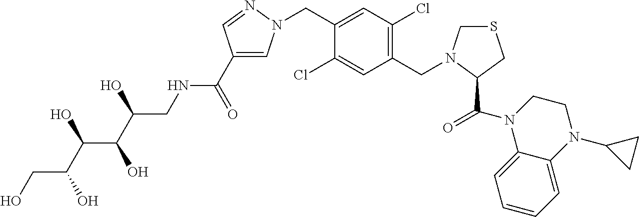 [SMILES: O=C(NC[C@H](O)[C@@H](O)[C@H](O)[C@H](O)CO)c1cnn(Cc2cc(Cl)c(CN3CSC[C@H]3C(=O)N3CCN(C4CC4)c4ccccc43)cc2Cl)c1] | C | 734.21 | 735.31 [M +H]+ |
| 235 | 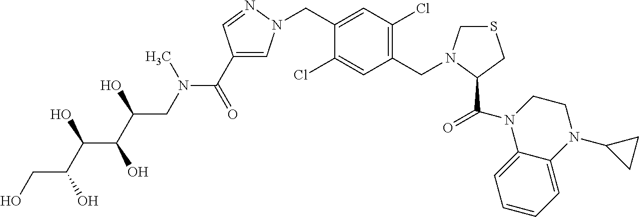 [SMILES: CN(C[C@H](O)[C@@H](O)[C@H](O)[C@H](O)CO)C(=O)c1cnn(Cc2cc(Cl)c(CN3CSC[C@H]3C(=O)N3CCN(C4CC4)c4ccccc43)cc2Cl)c1] | C | 748.22 | 749.35 [M +H]+ |
| 236 | 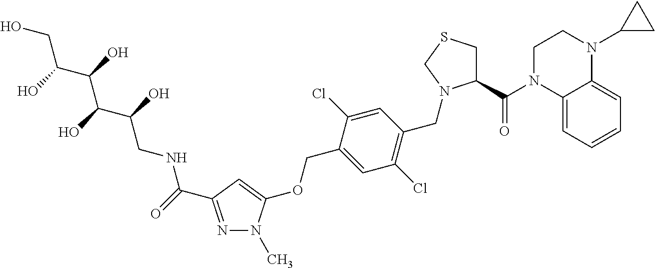 [SMILES: Cn1nc(C(=O)NC[C@H](O)[C@@H](O)[C@H](O)[C@H](O)CO)cc1OCc1cc(Cl)c(CN2CSC[C@H]2C(=O)N2CCN(C3CC3)c3ccccc32)cc1Cl] | C | 764.22 | 765.28 [M +H]+ |
| 237 | 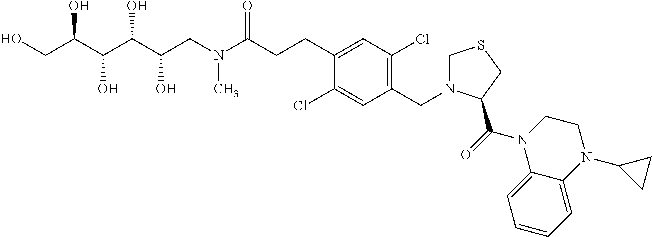 [SMILES: CN(C[C@H](O)[C@@H](O)[C@H](O)[C@H](O)CO)C(=O)CCc1cc(Cl)c(CN2CSC[C@H]2C(=O)N2CCN(C3CC3)c3ccccc32)cc1Cl] | Example 48 | 696.22 | 697.29 [M +H]+ |
| 238 | 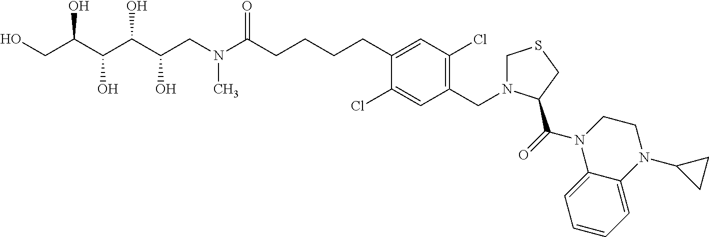 [SMILES: CN(C[C@H](O)[C@@H](O)[C@H](O)[C@H](O)CO)C(=O)CCCCc1cc(Cl)c(CN2CSC[C@H]2C(=O)N2CCN(C3CC3)c3ccccc32)cc1Cl] | E | 724.25 | 725.33 [M +H]+ |
| 239 | 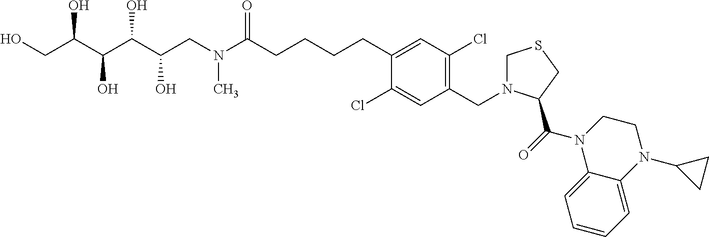 [SMILES: CN(C[C@H](O)[C@@H](O)[C@@H](O)[C@H](O)CO)C(=O)CCCCc1cc(Cl)c(CN2CSC[C@H]2C(=O)N2CCN(C3CC3)c3ccccc32)cc1Cl] | E | 724.25 | 725.25 [M +H]+ |
| 240 | 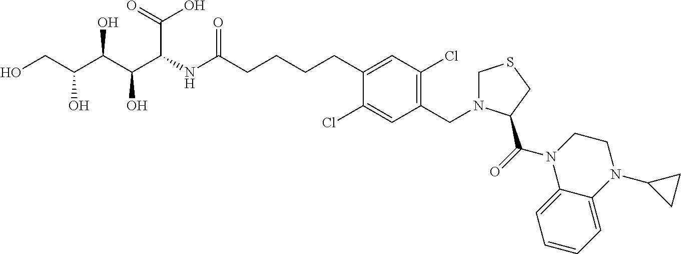 [SMILES: O=C(CCCCc1cc(Cl)c(CN2CSC[C@H]2C(=O)N2CCN(C3CC3)c3ccccc32)cc1Cl)N[C@@H](C(=O)O)[C@@H](O)[C@H](O)[C@H](O)CO] | E | 724.21 | 725.25 [M +H]+ |
| 241 | 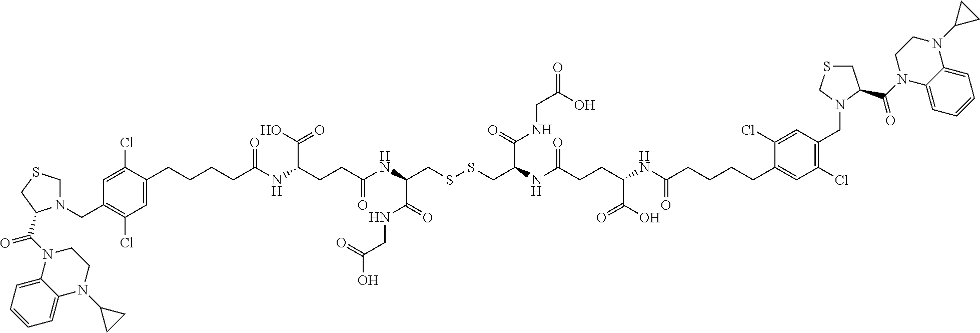 [SMILES: O=C(O)CNC(=O)[C@H](CSSC[C@H](NC(=O)CC[C@H](NC(=O)CCCCc1cc(Cl)c(CN2CSC[C@H]2C(=O)N2CCN(C3CC3)c3ccccc32)cc1Cl)C(=O)O)C(=O)NCC(=O)O)NC(=O)CC[C@H](NC(=O)CCCCc1cc(Cl)c(CN2CSC[C@H]2C(=O)N2CCN(C3CC3)c3ccccc32)cc1Cl)C(=O)O] | E | 1670.42 | 1674.95 [M +H]+ |
| 242 | 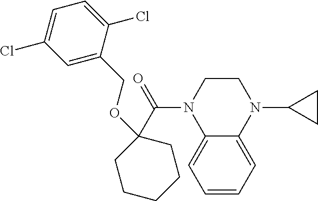 [SMILES: O=C(N1CCN(C2CC2)c2ccccc21)C1(OCc2cc(Cl)ccc2Cl)CCCCC1] | Example 8 | 458.15 | 459 [M +H]+ |
| 243 | 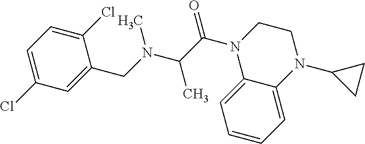 [SMILES: CC(C(=O)N1CCN(C2CC2)c2ccccc21)N(C)Cc1cc(Cl)ccc1Cl] | Example 7 | 417.14 | 418 [M +H]⁺ |
| 244 | 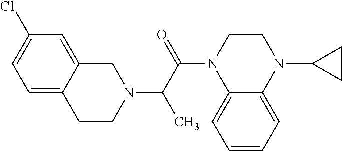 [SMILES: CC(C(=O)N1CCN(C2CC2)c2ccccc21)N1CCc2ccc(Cl)cc2C1] | Example 7 | 395.18 | 396.2 [M +H]⁺ |
| 245 | 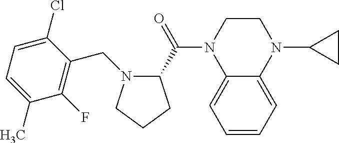 [SMILES: Cc1ccc(Cl)c(CN2CCC[C@H]2C(=O)N2CCN(C3CC3)c3ccccc32)c1F] | Example 12 | 427.18 | 428 [M +H]⁺ |
| 246 | 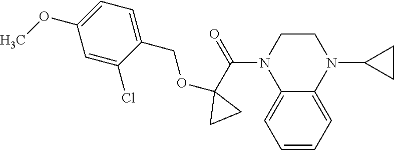 [SMILES: COc1ccc(COC2(C(=O)N3CCN(C4CC4)c4ccccc43)CC2)c(Cl)c1] | Example 9 | 412.16 | 413 [M +H]⁺ |
| 247 | 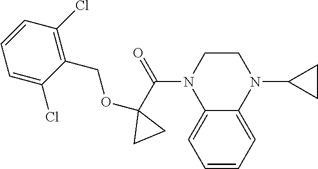 [SMILES: O=C(N1CCN(C2CC2)c2ccccc21)C1(OCc2c(Cl)cccc2Cl)CC1] | Example 9 | 416.11 | 417 [M +H]⁺ |
| 248 | 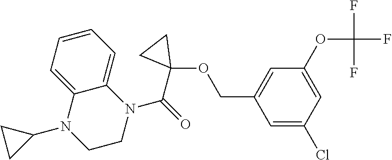 [SMILES: O=C(N1CCN(C2CC2)c2ccccc21)C1(OCc2cc(Cl)cc(OC(F)(F)F)c2)CC1] | Example 9 | 466.13 | 467.2 [M +H]⁺ |
| 249 | 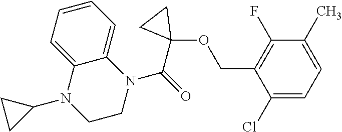 [SMILES: Cc1ccc(Cl)c(COC2(C(=O)N3CCN(C4CC4)c4ccccc43)CC2)c1F] | Example 9 | 414.15 | 415.2 [M +H]⁺ |
| 250 | 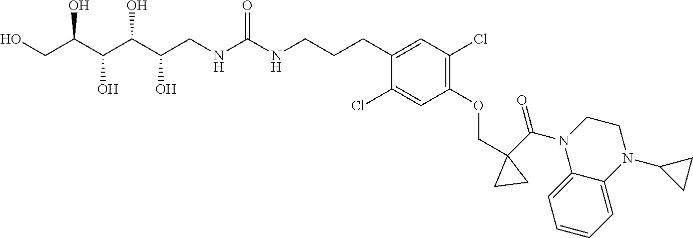 [SMILES: O=C(NCCCc1cc(Cl)c(OCC2(C(=O)N3CCN(C4CC4)c4ccccc43)CC2)cc1Cl)NC[C@H](O)[C@@H](O)[C@H](O)[C@H](O)CO] | E | 680.24 | 681.1 [M +H]⁺ |
| 251 | 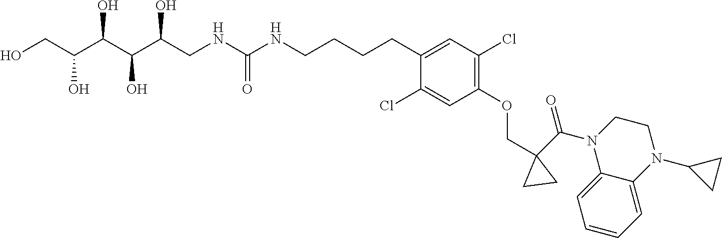 [SMILES: O=C(NCCCCc1cc(Cl)c(OCC2(C(=O)N3CCN(C4CC4)c4ccccc43)CC2)cc1Cl)NC[C@H](O)[C@@H](O)[C@H](O)[C@H](O)CO] | E | 694.25 | 695.1 [M +H]⁺ |
| 252 | 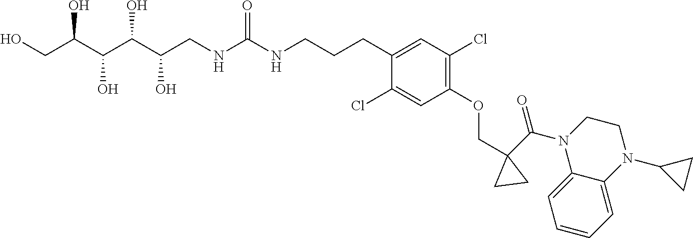 [SMILES: O=C(NCCCc1cc(Cl)c(OCC2(C(=O)N3CCN(C4CC4)c4ccccc43)CC2)cc1Cl)NC[C@H](O)[C@@H](O)[C@H](O)[C@H](O)CO] | E | 679.24 | 680.1 [M +H]⁺ |
| 253 | 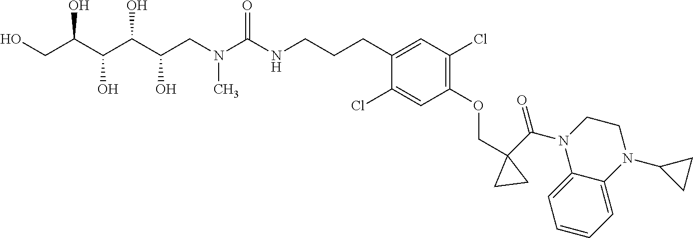 [SMILES: CN(C[C@H](O)[C@@H](O)[C@H](O)[C@H](O)CO)C(=O)NCCCc1cc(Cl)c(OCC2(C(=O)N3CCN(C4CC4)c4ccccc43)CC2)cc1Cl] | E | 693.26 | 693.4 [M +H]⁺ |
| 254 | 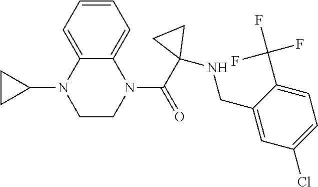 [SMILES: O=C(N1CCN(C2CC2)c2ccccc21)C1(NCc2cc(Cl)ccc2C(F)(F)F)CC1] | Example 26 | 449.15 | 450 [M +H]⁺ |
| 255 | 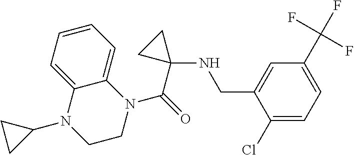 [SMILES: O=C(N1CCN(C2CC2)c2ccccc21)C1(NCc2cc(C(F)(F)F)ccc2Cl)CC1] | Example 26 | 449.15 | 450 [M +H]⁺ |
| 256 | 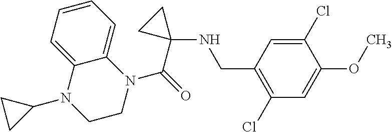 [SMILES: COc1cc(Cl)c(CNC2(C(=O)N3CCN(C4CC4)c4ccccc43)CC2)cc1Cl] | Example 26 | 445.13 | 446 [M +H]⁺ |
| 257 | 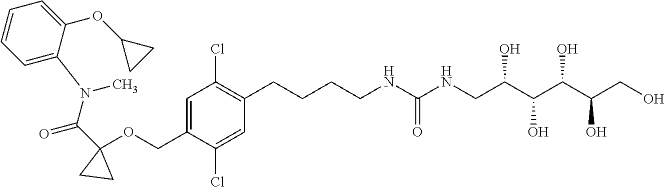 [SMILES: CN(C(=O)C1(OCc2cc(Cl)c(CCCCNC(=O)NC[C@H](O)[C@@H](O)[C@H](O)[C@H](O)CO)cc2Cl)CC1)c1ccccc1OC1CC1] | E | 683.24 | 684 [M +H]⁺ |
| 258 | 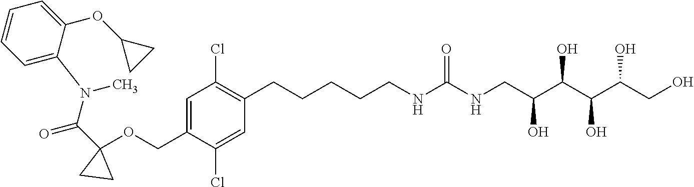 [SMILES: CN(C(=O)C1(OCc2cc(Cl)c(CCCCCNC(=O)NC[C@H](O)[C@@H](O)[C@H](O)[C@H](O)CO)cc2Cl)CC1)c1ccccc1OC1CC1] | E | 697.25 | 698 [M +H]⁺ |
| 259 | 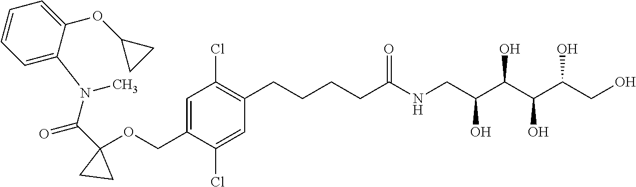 [SMILES: CN(C(=O)C1(OCc2cc(Cl)c(CCCCC(=O)NC[C@H](O)[C@@H](O)[C@H](O)[C@H](O)CO)cc2Cl)CC1)c1ccccc1OC1CC1] | E | 668.23 | 669 [M +H]⁺ |
| 260 | 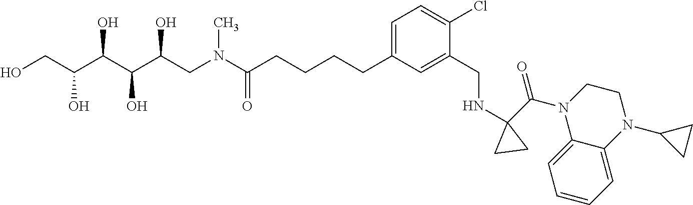 [SMILES: CN(C[C@H](O)[C@@H](O)[C@H](O)[C@H](O)CO)C(=O)CCCCc1ccc(Cl)c(CNC2(C(=O)N3CCN(C4CC4)c4ccccc43)CC2)c1] | E | 658.31 | 659.1 [M +H]⁺ |
| 261 | 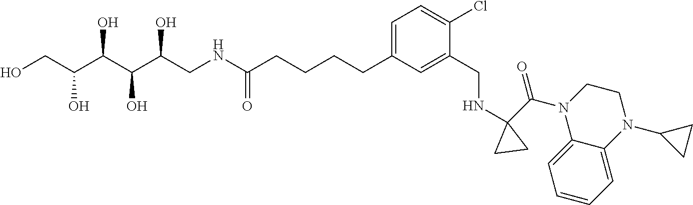 [SMILES: O=C(CCCCc1ccc(Cl)c(CNC2(C(=O)N3CCN(C4CC4)c4ccccc43)CC2)c1)NC[C@H](O)[C@@H](O)[C@H](O)[C@H](O)CO] | E | 644.30 | 645.5 [M +H]⁺ |
| 262 | 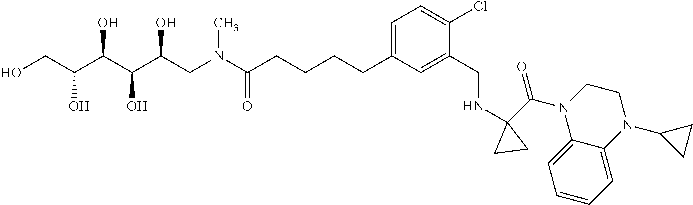 [SMILES: CN(C[C@H](O)[C@@H](O)[C@H](O)[C@H](O)CO)C(=O)CCCCc1ccc(Cl)c(CNC2(C(=O)N3CCN(C4CC4)c4ccccc43)CC2)c1] | E | 659.30 | 660.5 [M +H]⁺ |
| 263 | 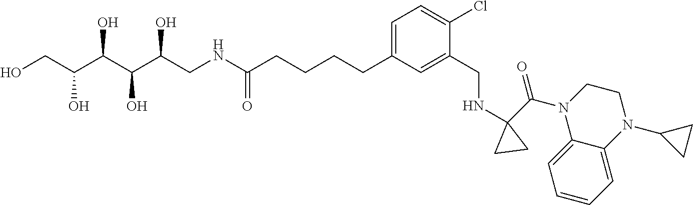 [SMILES: O=C(CCCCc1ccc(Cl)c(CNC2(C(=O)N3CCN(C4CC4)c4ccccc43)CC2)c1)NC[C@H](O)[C@@H](O)[C@H](O)[C@H](O)CO] | E | 645.28 | 646.1 [M +H]⁺ |
| 264 | 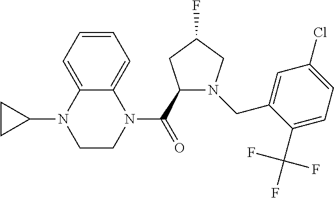 [SMILES: O=C([C@H]1C[C@H](F)CN1Cc1cc(Cl)ccc1C(F)(F)F)N1CCN(C2CC2)c2ccccc21] | Example 3 | 481.15 | 482.0 [M +H]⁺ |
| 265 | 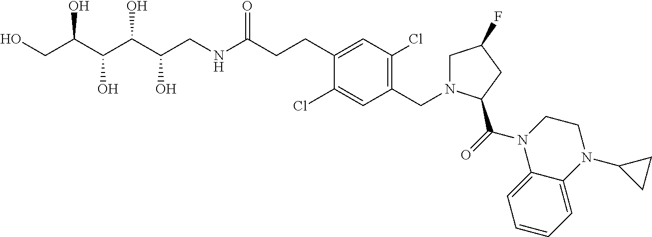 [SMILES: O=C(CCc1cc(Cl)c(CN2C[C@@H](F)C[C@H]2C(=O)N2CCN(C3CC3)c3ccccc32)cc1Cl)NC[C@H](O)[C@@H](O)[C@H](O)[C@H](O)CO] | Example 48 | 682.23 | 683.2 [M +H]⁺ |
| 266 | 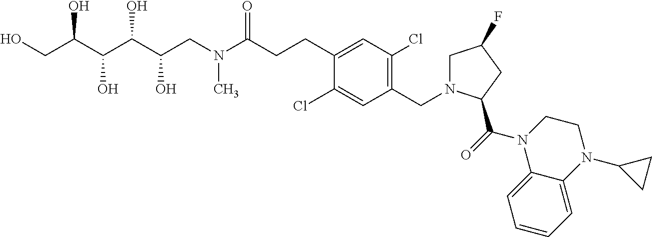 [SMILES: CN(C[C@H](O)[C@@H](O)[C@H](O)[C@H](O)CO)C(=O)CCc1cc(Cl)c(CN2C[C@@H](F)C[C@H]2C(=O)N2CCN(C3CC3)c3ccccc32)cc1Cl] | Example 48 | 696.25 | 697.4 [M +H]⁺ |
| 267 | 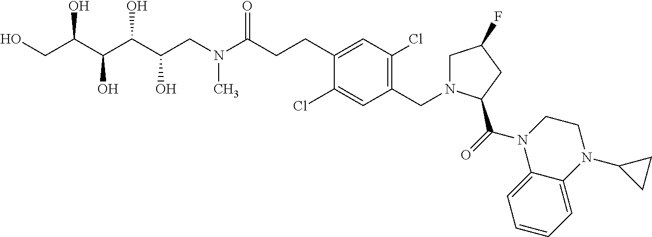 [SMILES: CN(C[C@H](O)[C@@H](O)[C@@H](O)[C@H](O)CO)C(=O)CCc1cc(Cl)c(CN2C[C@@H](F)C[C@H]2C(=O)N2CCN(C3CC3)c3ccccc32)cc1Cl] | Example 48 | 696.25 | 697.2 [M +H]⁺ |
| 268 | 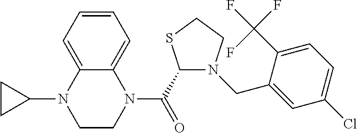 [SMILES: O=C([C@@H]1SCCN1Cc1cc(Cl)ccc1C(F)(F)F)N1CCN(C2CC2)c2ccccc21] | Example 21 | 481.12 | 504 [M +Na]⁺ |
| 269 | 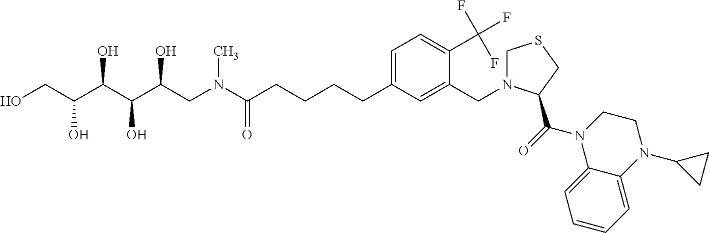 [SMILES: CN(C[C@H](O)[C@@H](O)[C@H](O)[C@H](O)CO)C(=O)CCCCc1ccc(C(F)(F)F)c(CN2CSC[C@H]2C(=O)N2CCN(C3CC3)c3ccccc32)c1] | Example 172 | 724.31 | 725.5 [M +H]⁺ |
| 270 | 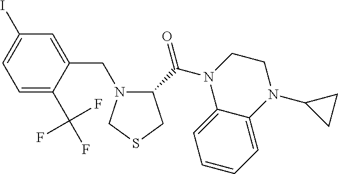 [SMILES: O=C([C@@H]1CSCN1Cc1cc(I)ccc1C(F)(F)F)N1CCN(C2CC2)c2ccccc21] | Example 164 | 573.06 | 574 [M +H]⁺ |
| 271 | 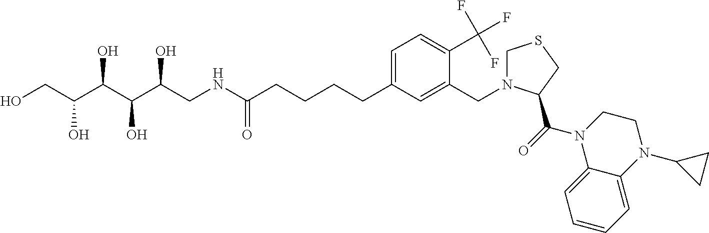 [SMILES: O=C(CCCCc1ccc(C(F)(F)F)c(CN2CSC[C@H]2C(=O)N2CCN(C3CC3)c3ccccc32)c1)NC[C@H](O)[C@@H](O)[C@H](O)[C@H](O)CO] | E | 710.30 | 711.5 [M +H]⁺ |
| 272 | 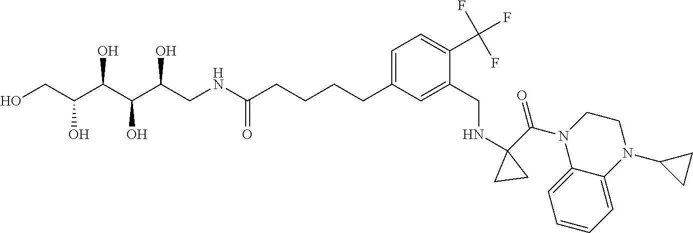 [SMILES: O=C(CCCCc1ccc(C(F)(F)F)c(CNC2(C(=O)N3CCN(C4CC4)c4ccccc43)CC2)c1)NC[C@H](O)[C@@H](O)[C@H](O)[C@H](O)CO] | E | 678.32 | 679 [M +H]⁺ |
| 273 | 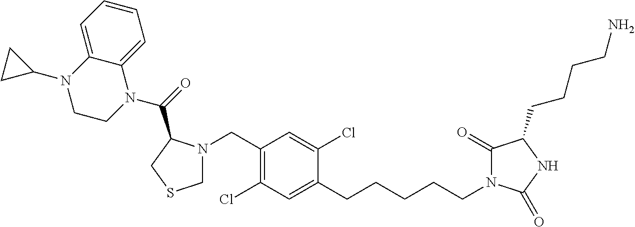 [SMILES: NCCCC[C@@H]1NC(=O)N(CCCCCc2cc(Cl)c(CN3CSC[C@H]3C(=O)N3CCN(C4CC4)c4ccccc43)cc2Cl)C1=O] | E | 686.26 | 687.4 [M +H]⁺ |
| 274 | 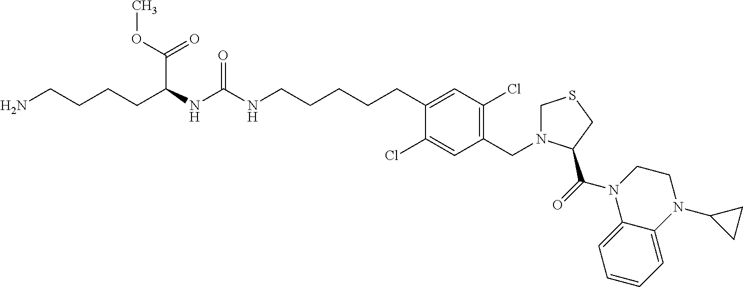 [SMILES: COC(=O)[C@H](CCCCN)NC(=O)NCCCCCc1cc(Cl)c(CN2CSC[C@H]2C(=O)N2CCN(C3CC3)c3ccccc32)cc1Cl] | E | 718.28 | 719.5 [M +H]⁺ |
| 275 | 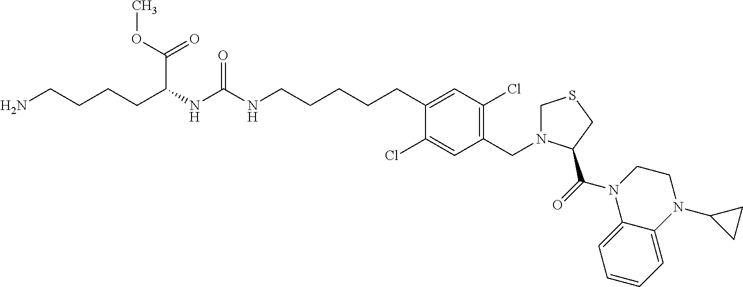 [SMILES: COC(=O)[C@@H](CCCCN)NC(=O)NCCCCCc1cc(Cl)c(CN2CSC[C@H]2C(=O)N2CCN(C3CC3)c3ccccc32)cc1Cl] | E | 718.28 | 719 [M +H]⁺ |
| 276 | 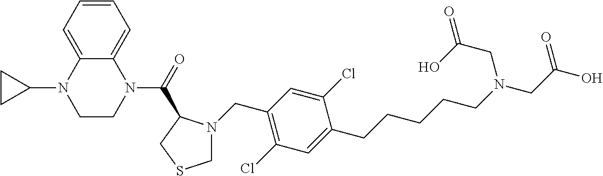 [SMILES: O=C(O)CN(CCCCCc1cc(Cl)c(CN2CSC[C@H]2C(=O)N2CCN(C3CC3)c3ccccc32)cc1Cl)CC(=O)O] | E | 648.19 | 649.4 [M +H]⁺ |
| 277 | 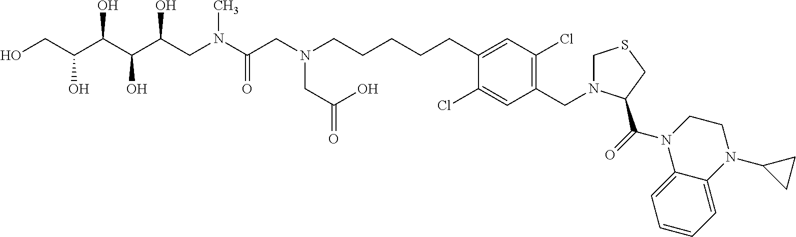 [SMILES: CN(C[C@H](O)[C@@H](O)[C@H](O)[C@H](O)CO)C(=O)CN(CCCCCc1cc(Cl)c(CN2CSC[C@H]2C(=O)N2CCN(C3CC3)c3ccccc32)cc1Cl)CC(=O)O] | E | 825.29 | 826 [M +H]⁺ |
| 278 | 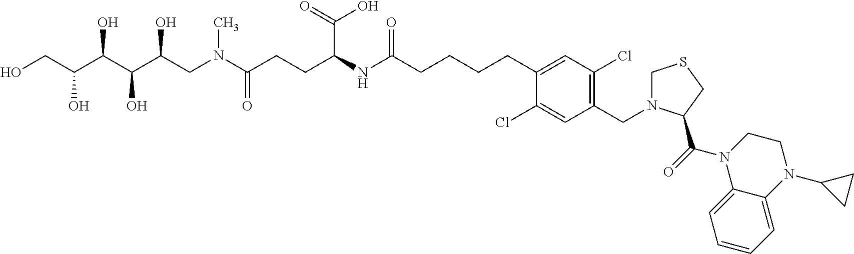 [SMILES: CN(C[C@H](O)[C@@H](O)[C@H](O)[C@H](O)CO)C(=O)CC[C@H](NC(=O)CCCCc1cc(Cl)c(CN2CSC[C@H]2C(=O)N2CCN(C3CC3)c3ccccc32)cc1Cl)C(=O)O] | E | 853.29 | 854 [M +H]⁺ |
| 279 | 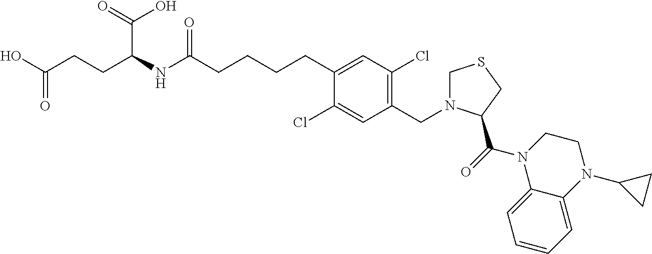 [SMILES: O=C(O)CC[C@H](NC(=O)CCCCc1cc(Cl)c(CN2CSC[C@H]2C(=O)N2CCN(C3CC3)c3ccccc32)cc1Cl)C(=O)O] | E | 676.19 | 677.1 [M +H]⁺ |
| 280 | 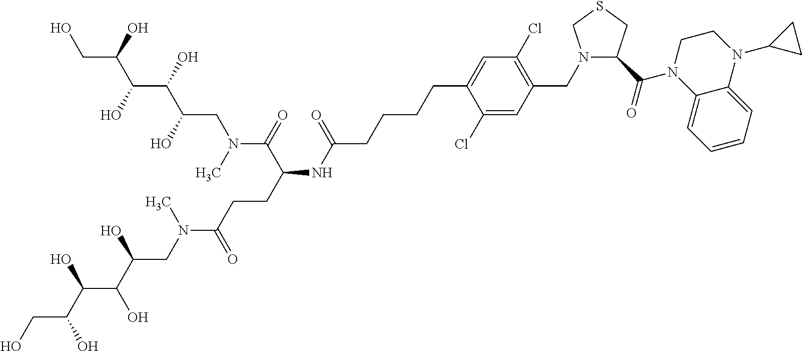 [SMILES: CN(C[C@H](O)C(O)[C@H](O)[C@H](O)CO)C(=O)CC[C@H](NC(=O)CCCCc1cc(Cl)c(CN2CSC[C@H]2C(=O)N2CCN(C3CC3)c3ccccc32)cc1Cl)C(=O)N(C)C[C@H](O)[C@@H](O)[C@H](O)[C@H](O)CO] | E | 1030.39 | 1031 [M +H]⁺ |
| 281 | 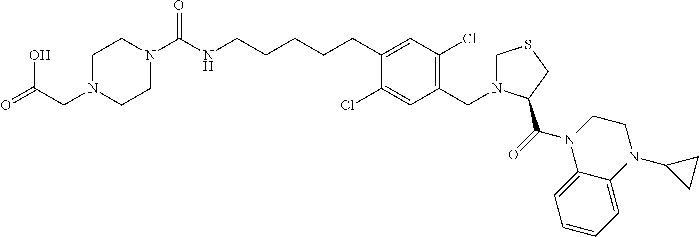 [SMILES: O=C(O)CN1CCN(C(=O)NCCCCCc2cc(Cl)c(CN3CSC[C@H]3C(=O)N3CCN(C4CC4)c4ccccc43)cc2Cl)CC1] | E | 702.25 | 703 [M +H]⁺ |
| 282 | 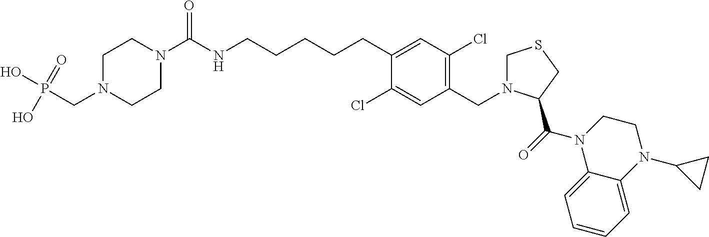 [SMILES: O=C(NCCCCCc1cc(Cl)c(CN2CSC[C@H]2C(=O)N2CCN(C3CC3)c3ccccc32)cc1Cl)N1CCN(CP(=O)(O)O)CC1] | E | 738.23 | 739 [M +H]⁺ |
| 283 | 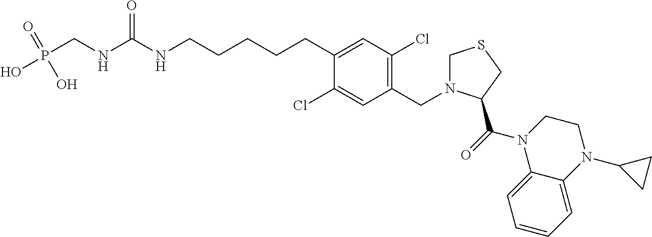 [SMILES: O=C(NCCCCCc1cc(Cl)c(CN2CSC[C@H]2C(=O)N2CCN(C3CC3)c3ccccc32)cc1Cl)NCP(=O)(O)O] | E | 669.17 | 670.3 [M +H]⁺ |
| 284 | 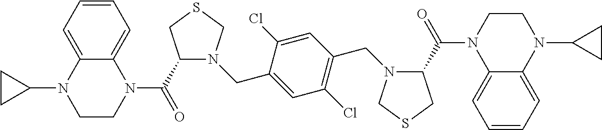 [SMILES: O=C([C@@H]1CSCN1Cc1cc(Cl)c(CN2CSC[C@H]2C(=O)N2CCN(C3CC3)c3ccccc32)cc1Cl)N1CCN(C2CC2)c2ccccc21] | Isolated as a side product from the synthesis of Example 159 | 748.22 | 749 [M +H]⁺ |
| 285 | 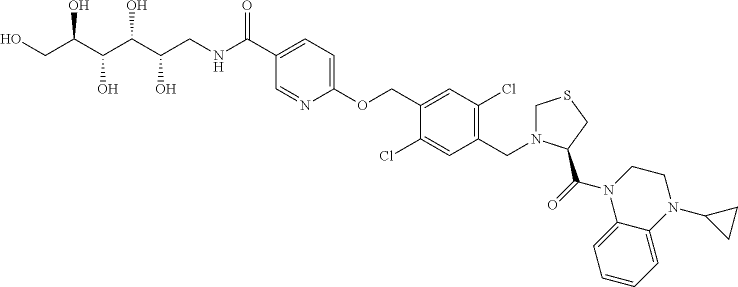 [SMILES: O=C(NC[C@H](O)[C@@H](O)[C@H](O)[C@H](O)CO)c1ccc(OCc2cc(Cl)c(CN3CSC[C@H]3C(=O)N3CCN(C4CC4)c4ccccc43)cc2Cl)nc1] | C | 761.21 | 762.4 [M +H]⁺ |
| 286 | 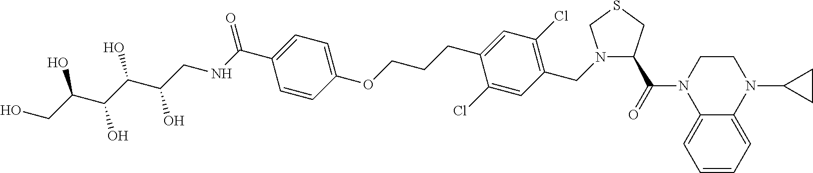 [SMILES: O=C(NC[C@H](O)[C@@H](O)[C@H](O)[C@H](O)CO)c1ccc(OCCCc2cc(Cl)c(CN3CSC[C@H]3C(=O)N3CCN(C4CC4)c4ccccc43)cc2Cl)cc1] | E | 788.24 | 789.2 [M +H]⁺ |
| 287 | 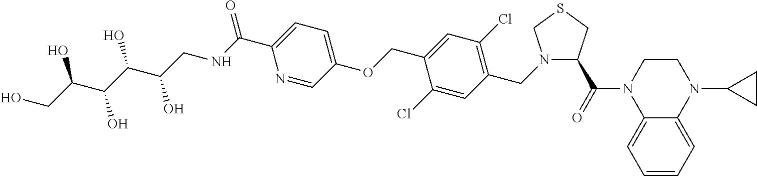 [SMILES: O=C(NC[C@H](O)[C@@H](O)[C@H](O)[C@H](O)CO)c1ccc(OCc2cc(Cl)c(CN3CSC[C@H]3C(=O)N3CCN(C4CC4)c4ccccc43)cc2Cl)cn1] | C | 761.21 | 764.2 [M +H]⁺ |
| 288 | 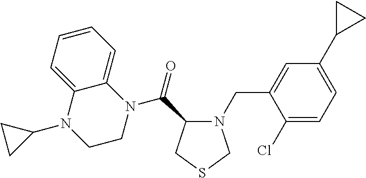 [SMILES: O=C([C@@H]1CSCN1Cc1cc(C2CC2)ccc1Cl)N1CCN(C2CC2)c2ccccc21] | Example 183 | 453.16 | 453 [M]⁺ |
| 289 | 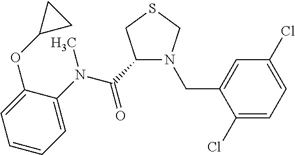 [SMILES: CN(C(=O)[C@@H]1CSCN1Cc1cc(Cl)ccc1Cl)c1ccccc1OC1CC1] | Example 82 | 436.08 | 437 [M +H]⁺ |
| 290 | 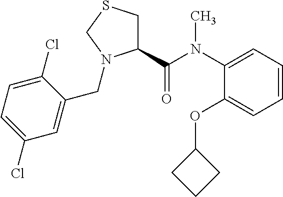 [SMILES: CN(C(=O)[C@@H]1CSCN1Cc1cc(Cl)ccc1Cl)c1ccccc1OC1CCC1] | Example 39 | 450.09 | 451 [M +H]⁺ |
| 291 | 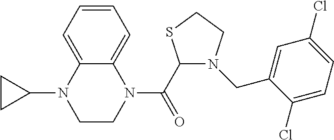 [SMILES: O=C(C1SCCN1Cc1cc(Cl)ccc1Cl)N1CCN(C2CC2)c2ccccc21] | Example 12 | 447.09 | 448.1 [M +H]⁺ |

### Example 292

### Cell-Based TGR5 Assays

Two primary cell based screens were performed. The first utilized HEK293 cells stably transfected to heterologously express human TGR5. The second screen used the human caecum carcinoma cell line NCI-H716 which endogenously expresses human TGR5. In both assays, cells are treated with candidate TGR5 activators and assessed for the increased intercellular levels of cAMP.

HEK293 cells were transfected with a vector that expresses a gene encoding human TGR5, and a stable cell line was isolated using drug selection following standard techniques. Cells were grown overnight at 37° C./5% CO₂ prior to assay. NCI-H716 were grown in culture dishes coated with Matrigel (Becton Dickinson) per the supplier's instructions and grown at 37° C./5% CO₂ for 48h prior to assay.

TGR5-mediated cAMP generation was measured using a homogeneous time resolved fluorescence (HTRF) detection method (Cisbio). Test compounds were dissolved in DMSO to a final concentration of 10 mM. Serial 3-fold dilutions of the stock solution were made in DMSO, and these solutions were diluted 100-fold into Hanks Balanced Salt Solution supplemented with 10 mM HEPES pH 7.4 and 0.5 mM isobutyl methylxanthine (IBMX). Prior to assay, culture medium was replaced with fresh medium, and test compounds diluted in HBSS/HEPES/IBMX were added to the cells and incubated at 37° C. for 30 minutes. Each compound was tested in duplicate at 12 concentrations ranging from 0.05 nM to 10 μM HEK293/hTGR5) or 22 nM to 50 μM (NCI H716).

Following incubation with test compounds, cAMP was detected through the successive addition of cAMP labeled with the modified allophyocyanin dye d2 (cAMP-d2) and cryptate-labeled anti-cAMP in lysis buffer, and reading HTRF per the manufacturer's instructions. A standard curve was used to convert the raw HTRF data into [cAMP]. The concentration of cAMP was plotted against log [test compound] and the resulting curves were fit to a 3-parameter logistical equation using GraphPad Prism to determine pEC₅₀ (the negative log of the EC₅₀) and the magnitude of the response. pEC₅₀ values are reported in the table below. The magnitude of the maximum response was typically between 50 and 200% of the maximum response elicited by a benchmark compound that had a maximum response similar to that elicited by lithocholic acid.

The results of this assay are set forth in Table 2.

TABLE 2
pEC₅₀ Values of Representative Compounds*

| | | pEC₅₀ endogenously |
|---|---|---|
| | | expressed TGR5 in |
| Example # | pEC₅₀ Human TGR5 | NCI H716 |
| 1 | B | B |
| 2 | B | B |
| 3 | A | B |
| 4 | C | E |
| 5 | A | B |
| 6 | B | C |
| 7 | B | C |
| 8 | A | B |
| 9 | A | B |
| 10 | C | C |
| 11 | C | C |
| 12 | B | B |
| 13 | C | C |
| 14 | C | B |
| 15 | C | C |
| 16 | C | C |
| 17 | B | C |
| 18 | C | C |
| 19 | C | C |
| 20 | B | B |
| 21 | A | B |
| 22 | B | C |
| 23 | B | C |
| 24 | C | C |
| 25 | C | C |
| 26 | A | A |
| 27 | B | C |
| 28 | C | C |
| 29 | A | B |
| 30 | C | C |
| 31 | A | B |
| 32 | A | B |
| 33 | C | C |
| 34 | B | C |
| 35 | C | C |
| 36 | A | B |
| 37 | B | C |
| 38 | A | A |
| 39 | D | C |
| 40 | A | B |
| 41 | A | B |
| 42 | B | B |
| 43 | A | A |
| 44 | C | C |
| 45 | B | C |
| 46 | A | B |
| 47 | D | C |
| 48 | B | C |
| 49 | B | C |
| 50 | B | C |
| 51 | B | C |
| 52 | B | C |
| 53 | C | C |
| 54 | B | C |
| 55 | B | C |
| 56 | A | A |
| 57 | C | C |
| 58 | C | C |
| 59 | B | C |
| 60 | B | B |
| 61 | B | B |
| 62 | C | C |
| 63 | C | C |
| 64 | C | C |
| 65 | C | C |
| 66 | C | C |
| 67 | C | C |
| 68 | C | C |
| 69 | C | C |
| 70 | C | C |
| 71 | B | B |
| 72 | A | A |
| 73 | A | A |
| 74 | B | E |
| 75 | B | B |
| 76 | A | B |
| 77 | A | C |
| 78 | A | B |
| 79 | B | C |
| 80 | A | B |
| 81 | B | C |
| 82 | B | C |
| 83 | B | C |
| 84 | A | B |
| 85 | B | C |
| 86 | D | E |
| 87 | A | B |
| 88 | A | A |
| 89 | B | C |
| 90 | A | B |
| 91 | B | B |
| 92 | C | C |
| 93 | B | B |
| 94 | C | C |
| 95 | A | C |
| 96 | A | A |
| 97 | A | A |
| 98 | A | B |
| 159 | A | |
| 160 | B | |
| 161 | B | |
| 162 | B | |
| 163 | A | |
| 164 | B | |
| 165 | A | |
| 166 | A | |
| 167 | B | |
| 168 | B | |
| 169 | A | |
| 170 | B | |
| 171 | B | |
| 172 | C | |
| 173 | B | |
| 174 | C | |
| 175 | A | |
| 176 | B | |
| 177 | A | |
| 178 | A | |
| 179 | A | B |
| 180 | A | B |
| 181 | A | |
| 182 | B | |
| 183 | A | |
| 184 | B | B |
| 185 | B | C |
| 186 | B | C |
| 187 | B | C |
| 188 | B | B |
| 189 | A | B |
| 190 | C | |
| 191 | A | B |
| 192 | A | |
| 193 | A | |
| 194 | A | |
| 195 | B | |
| 196 | B | |
| 197 | C | |
| 198 | A | |
| 199 | B | |
| 200 | B | |
| 201 | B | |
| 202 | B | |
| 203 | B | |
| 204 | B | |
| 205 | B | |
| 206 | A | |
| 207 | B | |
| 208 | A | |
| 209 | C | |
| 210 | A | |
| 211 | A | |
| 212 | C | C |
| 213 | B | C |
| 214 | B | B |
| 215 | B | C |
| 216 | B | C |
| 217 | B | C |
| 218 | A | A |
| 219 | A | A |
| 220 | A | A |
| 221 | B | |
| 222 | A | |
| 223 | B | |
| 224 | B | |
| 225 | A | |
| 226 | B | |
| 227 | A | |
| 228 | A | |
| 229 | B | |
| 230 | B | |
| 231 | B | |
| 232 | C | |
| 233 | B | |
| 234 | A | |
| 235 | B | |
| 236 | A | |
| 237 | A | B |
| 238 | A | |
| 239 | A | |
| 240 | A | |
| 241 | A | |
| 242 | B | E |
| 243 | B | C |
| 244 | C | C |
| 245 | C | C |
| 246 | B | C |
| 247 | C | C |
| 248 | C | C |
| 249 | C | C |
| 250 | B | |
| 251 | B | |
| 252 | B | |
| 253 | B | |
| 254 | A | A |
| 255 | A | A |
| 256 | B | B |
| 257 | A | B |
| 258 | A | |
| 259 | A | |
| 260 | B | |
| 261 | B | |
| 262 | C | |
| 263 | C | |
| 264 | B | |
| 265 | B | |
| 266 | C | |
| 267 | C | |
| 268 | A | B |
| 269 | C | |
| 270 | B | |
| 271 | C | |
| 272 | C | |
| 273 | B | |
| 274 | B | |
| 275 | B | |
| 276 | A | |
| 277 | B | |
| 278 | B | |
| 279 | A | |
| 280 | B | |
| 281 | A | |
| 282 | A | |
| 283 | B | |
| 284 | C | |
| 285 | A | |
| 286 | B | |
| 287 | B | |
| 288 | B | |
| 289 | B | |
| 290 | B | |
| 291 | C | |

*pEC₅₀ values are expressed as the following ranges: A is a pEC₅₀ of 7+, B is a pEC₅₀ of 6-6.9, C is a pEC₅₀ of 4.3-5.9, D is a pEC₅₀ of <5, E is a pEC₅₀ of <4.3

### Example 293

### In Vivo GLP-1 Secretion and Gallbladder Measurement

C57BL/6 male mice on regular chow had food removed in the morning and were dosed with vehicle (10% hydroxypropyl-β-cyclodextrin or 2% DMSO in 0.4% hydroxypropyl methylcellulose) or the test compound in vehicle to achieve a dose of 30 mg/kg. Eight hours later, each mouse was heavily anesthetized with isoflurane, the peritoneal cavity was opened and the gallbladder, with its entire contents, was carefully excised and weighed. Blood was collected from the left ventricle of the heart and processed to plasma in EDTA-coated tubes containing aprotinin and DPPIV inhibitor for measurement of total GLP-1 (K150FCC; Meso Scales Discovery, Gaithersburg Md.).

As shown in Tables 2 and 3, of the compounds tested, 18 significantly increased GLP-1 levels, and of these 18, 8 had no significant effects on gallbladder weight/body weight.

TABLE 3
GLP-1 Levels

GLP-1 fold
change over vehicle

| Example # | Mean | SEM | Significance |
|---|---|---|---|
| 26 | 1.6 | 0.2 | ** |
| 96 | 1.9 | 0.3 | * |
| 97 | 1.7 | 0.2 | n.s. |
| 98 | 1.8 | 0.3 | * |
| 165 | 1.2 | 0.1 | n.s. |
| 166 | 1.5 | 0.1 | n.s. |
| 167 | 1.3 | 0.1 | n.s. |
| 169 | 1.5 | 0.2 | n.s. |
| 175 | 2.2 | 0.1 | * |
| 176 | 2.3 | 0.1 | * |
| 177 | 2.5 | 0.5 | * |
| 178 | 3.2 | 0.5 | * |
| 191 | 1.5 | 0.1 | * |
| 193 | 0.9 | 0.1 | n.s. |
| 198 | 1.5 | 0.2 | * |
| 208 | 1.3 | 0.1 | n.s. |
| 218 | 1.4 | 0.1 | * |
| 227 | 1.9 | 0.1 | * |
| 228 | 1.6 | 0.2 | * |
| 237 | 2.3 | 0.2 | * |
| 238 | 3.1 | 0.3 | * |
| 239 | 3.1 | 0.4 | * |
| 240 | 1.9 | 0.2 | * |
| 241 | 1.1 | 0.1 | n.s. |
| 258 | 1.1 | 0.2 | n.s. |
| 259 | 1.4 | 0.2 | n.s. |
| 276 | 1.5 | 0.1 | * |
| 278 | 1.4 | 0.2 | n.s. |
| 279 | 3.0 | 0.4 | * |
| 282 | 0.8 | 0.1 | n.s. |
| 283 | 1.3 | 0.1 | n.s. |
| 285 | 1.5 | 0.2 | n.s. |

* p < 0.05 vs. vehicle; One-way ANOVA followed by Dunnett's test
n.s. = non significant

TABLE 4
Gallbladder Weight

Gallbladder weight/
body weight fold
change over vehicle

| Example # | Mean | SEM | Significance |
|---|---|---|---|
| 26 | 1.6 | 0.1 | * |
| 96 | 0.8 | 0.1 | n.s. |
| 97 | 1.0 | 0.1 | n.s. |
| 98 | 1.1 | 0.1 | n.s. |
| 165 | 1.5 | 0.1 | n.s. |
| 166 | 1.2 | 0.2 | n.s. |
| 167 | 1.3 | 0.1 | n.s. |
| 169 | 1.9 | 0.2 | ** |
| 175 | 1.4 | 0.1 | * |
| 176 | 1.1 | 0.1 | n.s. |
| 177 | 1.2 | 0.1 | n.s. |
| 178 | 1.2 | 0.1 | * |
| 191 | 1.0 | 0.0 | n.s. |
| 193 | 1.1 | 0.1 | n.s. |
| 198 | 1.9 | 0.2 | * |
| 208 | 1.2 | 0.1 | n.s. |
| 218 | 1.0 | 0.1 | n.s. |
| 227 | 1.4 | 0.1 | * |
| 228 | 1.5 | 0.1 | * |
| 237 | 1.3 | 0.1 | n.s. |
| 238 | 1.4 | 0.1 | * |
| 239 | 1.4 | 0.2 | n.s. |
| 240 | 1.2 | 0.1 | * |
| 241 | 1.2 | 0.1 | n.s. |
| 258 | 1.1 | 0.1 | n.s. |
| 259 | 0.9 | 0.1 | n.s. |
| 276 | 1.6 | 0.1 | * |
| 278 | 1.3 | 0.1 | * |
| 279 | 2.4 | 0.2 | * |
| 282 | 0.8 | 0.1 | n.s. |
| 283 | 1.3 | 0.0 | * |
| 285 | 1.1 | 0.1 | n.s. |

* p < 0.05 vs. vehicle; One-way ANOVA followed by Dunnett's test
n.s. = non significant

### Example 294

### Determination of Compound Concentration in Gall Bladder

Compound concentrations in the gall bladder were determined as follows: each mouse was heavily anesthetized with isoflurane, the peritoneal cavity was opened and the gallbladder, with its entire contents, was carefully excised. After harvest, gall bladders were homogenized in 100 μL water using a micro-homogenizer. Samples of homogenate were diluted 1:5 in water and precipitated with three volumes of neat acetonitrile. After centrifugation, supernatants were analyzed by LC-MS/MS. The level of test compound present in gall bladder samples was interpolated from a standard curve for each individual compound prepared in a matrix of gallbladder homogenate from vehicle treated animals. Table 5 summarizes data collected for selected example compounds and shows compound concentration in gallbladders collected from the mice in example 293.

TABLE 5
Example Compound Concentration in Gallbladder at 8 Hours Post Dose

| | | | Number of Mice |
|---|---|---|---|
| | | Mean Example | with |
| | | Gallbladder | Gallbladder |
| | LLOQ | Concentration | Concentration < |
| Example | (μM) | (μM) | LLOQ |
| 26 | 0.012 | 0.03 | 0/8 |
| 96 | 0.075 | 1.34 | 0/8 |
| 97 | 0.073 | 0.52 | 0/8 |
| 98 | 0.036 | 25.76 | 0/8 |
| 165 | 0.012 | 0.6 | 0/8 |
| 166 | 0.012 | — | 8/8 |
| 167 | 0.012 | 3.3 | 0/8 |
| 169 | 0.012 | 1.2 | 0/8 |
| 175 | 0.003 | 0.06 | 0/8 |
| 176 | 0.013 | 0.1 | 0/8 |
| 177 | 0.014 | 0.1 | 0/7 |
| 178 | 0.007 | 0.3 | 0/8 |
| 191 | 0.003 | 0.1 | 0/8 |
| 193 | 0.015 | 0.9 | 0/8 |
| 198 | 0.037 | 1.3 | 0/8 |
| 208 | 0.003 | 0.2 | 0/7 |
| 218 | 0.004 | 0.1 | 0/8 |
| 227 | 0.007 | 1.73 | 0/7 |
| 228 | 0.003 | 0.60 | 0/8 |
| 237 | 0.004 | 1.6 | 1/8 |
| 238 | 0.034 | 4.4 | 0/8 |
| 239 | 0.003 | — | 8/8 |
| 240 | 0.003 | 3.92 | 0/8 |
| 241 | 0.060 | — | 8/8 |
| 258 | 0.004 | 0.7 | 0/8 |
| 259 | 0.004 | 0.1 | 0/8 |
| 276 | 0.077 | 4.7 | 0/8 |
| 278 | 0.012 | 3.1 | 0/8 |
| 279 | 0.738 | 30.8 | 0/7 |
| 282 | 0.007 | 0.08 | 0/7 |
| 283 | 0.037 | 3.06 | 0/8 |
| 285 | 0.003 | 0.04 | 0/8 |

LLOQ = Lower Limit of Quantification

### Example 295

### Determination of Compound Plasma Concentration

Blood samples collected as described in Example 293 were processed to plasma by centrifugation. Plasma samples were treated with acetonitrile containing an internal standard, precipitated proteins removed by centrifugation. Supernatants were analyzed by LC-MS/MS and compound concentrations were determined by interpolated from a standard curve prepared in plasma. Table 6 summarizes data collected for selected example compounds for compound concentration in plasma collected in example 293.

TABLE 6
Example Compound Concentration in Plasma at 8 Hours Post Dose

| | | | Number of |
|---|---|---|---|
| | | Mean Example | Mice with |
| | | Plasma | Plasma |
| | LLOQ | Concentration | Concentration < |
| Example | (ng/mL) | (ng/mL) | LLOQ |
| 26 | 0.5 | 9.1 | 0/8 |
| 96 | 2 | — | 8/8 |
| 97 | 0.5 | 0.5 | 7/8 |
| 98 | 0.5 | 1 | 4/8 |
| 165 | 0.5 | 2.2 | 2/8 |
| 166 | 0.5 | 1.4 | 2/8 |
| 167 | 0.5 | 2.3 | 5/8 |
| 169 | 1 | 2.1 | 4/8 |
| 175 | 0.5 | 0.5 | 7/8 |
| 176 | 2 | — | 8/8 |
| 177 | 0.5 | 0.7 | 4/7 |
| 178 | 1 | — | 8/8 |
| 191 | 0.5 | — | 8/8 |
| 193 | 1 | 5.2 | 5/8 |
| 198 | 1 | 3.6 | 0/8 |
| 208 | 0.5 | — | 7/7 |
| 218 | 0.5 | 0.7 | 2/8 |
| 227 | 0.5 | 0.6 | 7/8 |
| 228 | 0.5 | — | 8/8 |
| 237 | 0.5 | 0.9 | 7/8 |
| 238 | 1 | 1.2 | 3/8 |
| 239 | 2 | 2.0 | 6/8 |
| 240 | 0.5 | 28.1 | 0/8 |
| 241 | 10 | — | 8/8 |
| 258 | 1 | 9.4 | 7/8 |
| 259 | 1 | 1.1 | 6/8 |
| 276 | 2 | 7.6 | 0/8 |
| 278 | 2 | 2.3 | 5/8 |
| 279 | 1 | 2.0 | 0/8 |
| 282 | 0.5 | 2.8 | 0/8 |
| 283 | 10 | 24.8 | 7/8 |
| 285 | 0.5 | 0.7 | 2/8 |

LLOQ = Lower Limit of Quantification

### Example 296

### Counter Screens Vs. Ileal Bile Acid Transporter (IBAT) and Farnesoid X Receptor (FXR)

IBAT: HEK293 cells were transfected with a vector that expresses a gene encoding human IBAT and inhibition of the uptake of [Taurine⁻³H]taurocholic acid was measured in a manner similar to that described by Craddock (Craddock_{—}1998). IBAT-transfected cells were overlayed with uptake buffer (10 mM HEPES, 116 mM sodium chloride, 5.3 mM KCl, 1.8 mM CaCl₂, 11 mM glucose, 1.1 mM KH₂PO₄, pH 7.4) containing 10 μM [³H]taurocholic acid (American Radiolabeled Chemicals, St. Louis, Mo.) and 0 to 26 uM test compound. Following a 40-min incubation, the solution was removed, and the cells were washed twice with uptake buffer. Cells were lysed by addition of 20 μL 0.1% Tween 80 followed by 100 μL scintillation fluid and counted using a TopCount (Perkin Elmer).

Taurocholate, deoxycholate, and chenodeoxycholic acid each inhibited uptake of [³H]taurocholic acid with potency similar to that reported by Craddock (Craddock_{—}1998); none of the test compounds inhibited [³H]taurocholic acid uptake (pIC50<4.6; Table 7).

FXR: The ability of test compounds to activate FXR (NR1H4) was measured using a cell-based assay kit obtained from Indigo Biosciences (State College, Pa.). Cells expressing human FXR and a FXR-responsive luciferase reporter gene were grown in duplicate in the presence of 0.4 to 50 μM test compound in buffer according to the manufacturer's instructions. The assay was benchmarked using GW 4064 and chenodeoxycholic acid, which showed pEC₅₀ values of 6.8 and ˜4-4.5, respectively, similar to literature reports (Maloney, 2000). None of the compounds tested showed any inhibition of human FXR (pEC50<4.3; Table 7).

### REFERENCES

Craddock, A. L., Love, M. W., Daniel, R. W., Kirby, L. C., Walters, H. C., Wong, M. H. and Dawson, P. A., 1998, Expression and transport properties of the human ileal and renal sodium-dependent bile acid transporter: Am. J. Gastrointest. Liver. Physiol. v.274, p. G157-69.
Maloney, P. R. et al., 2000, Identification of a chemical tools for the orphan nuclear receptor FXR: J. Med. Chem. v. 43, no. 16, p. 2971-4.

TABLE 7
pEC₅₀ Values of Representative Compounds**

| Example # | human IBAT | human FXR |
|---|---|---|
| 1 | G | |
| 2 | G | |
| 3 | G | |
| 4 | G | |
| 6 | G | |
| 8 | G | |
| 9 | G | E |
| 10 | G | |
| 11 | G | |
| 12 | G | |
| 13 | G | |
| 14 | G | |
| 15 | G | |
| 16 | G | |
| 17 | G | |
| 18 | G | |
| 19 | G | |
| 20 | G | |
| 21 | G | |
| 22 | G | |
| 23 | G | |
| 24 | G | |
| 25 | G | |
| 26 | G | E |
| 27 | G | |
| 28 | G | |
| 29 | G | |
| 31 | G | |
| 32 | | E |
| 34 | | E |
| 36 | G | |
| 37 | G | |
| 40 | G | |
| 56 | G | |
| 57 | G | |
| 61 | G | |
| 62 | G | |
| 63 | G | |
| 64 | G | |
| 65 | G | |
| 66 | G | |
| 73 | | E |
| 75 | | E |
| 80 | | E |
| 88 | | E |
| 175 | G | |
| 176 | G | E |
| 177 | G | E |
| 178 | G | E |
| 193 | G | |
| 198 | G | |
| 218 | G | |
| 228 | G | |
| 238 | G | |
| 240 | G | |
| 241 | G | |

**pEC₅₀ values are expressed as the following ranges: A is a pEC₅₀ of 7+, B is a pEC₅₀ of 6-6.9, C is a pEC₅₀ of 4.3-5.9, D is a pEC₅₀ of <5, E is a pEC₅₀ of <4.3, F is a pEC₅₀ of <5.5, G is a pEC₅₀ of <4.6.

### Example 297

### In Vivo Gallbladder Measurement 16 Hours Post-Dose

C57BL/6 male mice on regular chow had food removed (to prevent gallbladder emptying) and were dosed in the early evening with vehicle (2% DMSO in 0.4% hydroxypropyl methylcellulose) or 30 mg/kg of examples 176, 177, or 178 formulated in vehicle. The next morning (˜16 h post-dose), mice were heavily anesthetized with isoflurane, the peritoneal cavity was opened and gallbladders (with contents) were carefully excised and weighed. Compound levels in gallbladder were analyzed as described in Example 293.

Example compounds 176 and 178 had no significant effect on gallbladder weight/body weight, whereas example 177 did (Table 8).

TABLE 8
Gallbladder Weight 16 Hours Post Dose

Gallbladder weight/
body weight fold
change over vehicle

| Example # | Mean | SEM | Significance |
|---|---|---|---|
| 176 | 0.98 | 0.10 | n.s. |
| 177 | 1.85 | 0.16 | * |
| 178 | 0.94 | 0.08 | n.s. |

* p < 0.05 vs. vehicle; One-way ANOVA followed by Dunnett's test
n.s. = non significant

TABLE 9
Example Compound Concentration in Gallbladder at 16 Hours Post Dose

| | | Mean Example | Number of Mice |
|---|---|---|---|
| | | Gallbladder | with Gallbladder |
| | LLOQ | Concentration | Concentration < |
| Example | (μM) | (μM) | LLOQ |
| 83 | 0.013 | 0.03 | 0/6 |
| 85 | 0.004 | 0.20 | 0/6 |

LLOQ = Lower Limit of Quantification

### Example 298

### In Vivo Measurement of Gallbladder Emptying

CD-1 female mice on regular chow had food removed (to prevent gallbladder emptying) in the late evening. The next morning (˜16 h later), groups of mice were dosed orally with vehicle (10% hydroxypropyl-β-cyclodextrin; 2 groups), devazepide in water (1 mg/kg; a CCK receptor antagonist), or examples 176 and 178 (30 mg/kg in vehicle). One hour later, 1 group of vehicle-treated mice was dosed orally with saline, and all other groups were dosed orally with lyophilized egg yolk [0.75 mL; 30% (wt/vol) reconstituted in saline for induction of CCK-mediated gallbladder emptying]. Fifteen minutes later, mice were heavily anesthetized with isoflurane, the peritoneal cavity was opened and gallbladders (with contents) were carefully excised and weighed.

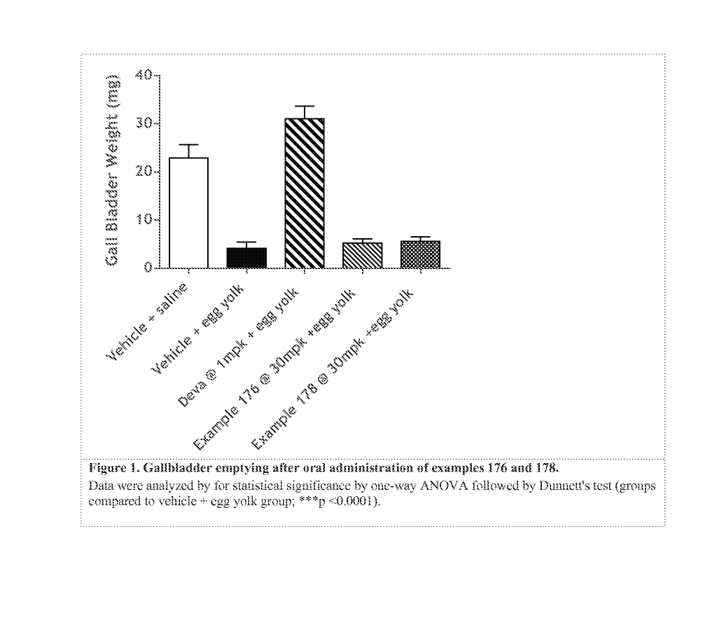

Examples 176 and 178 did not inhibit CCK-mediated gallbladder emptying, whereas devazepide, as expected, did (FIG. 1).

### Example 299

### Time Course of In Vivo Induction of GLP-1 and PYY

C57BL/6 male mice on regular chow received an oral dose of vehicle (2% DMSO in 0.4% hydroxypropyl methylcellulose) or examples 176 or 178 (at 30 mg/kg). Food was removed prior to/or at the time of dosing: 4, 8, 12, and 16 h groups fasted for 7, 8, 12 and 16 h, respectively. At the appropriate time after dosing, mice were heavily anesthetized with isoflurane, and blood was collected from the left ventricle of the heart and processed to plasma as described in Example 293 for measurement of total (t) GLP-1 and, additionally, total (t)PYY (N45ZA-1; Meso Scales Discovery, Gaithersburg Md.).

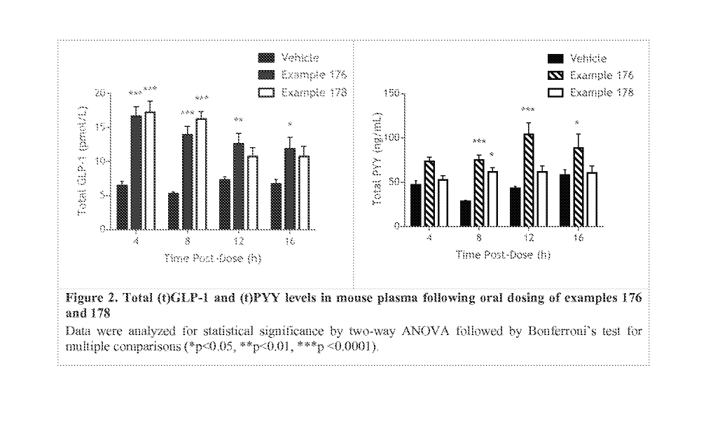

Examples 176 and 178 caused a sustained induction of tGLP-1 and tPYY levels following a single oral dose. tGLP-1 was significantly elevated at 4, 8, 12, and 16 h and 4 and 8 h for examples 176 and 178, respectively (FIG. 2, panel A), and tPYY levels were significantly elevated at 8, 12, and 16 h and 8 h for Examples 176 and 178, respectively (FIG. 2, panel B).

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, are incorporated herein by reference, in their entirety. Aspects of the embodiments can be modified, if necessary to employ concepts of the various patents, applications and publications to provide yet further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A compound having the following structure (XVIIa): 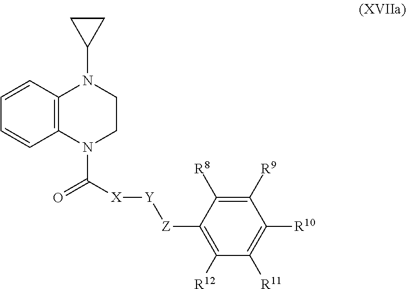
or a stereoisomer, tautomer, pharmaceutically acceptable salt or prodrug thereof, wherein:
X is CR⁵⁰R⁵¹ wherein:
R⁵⁰ and R⁵¹ are the same or different and independently selected from H and C₁₋₇-alkyl, or
R⁵⁰ and R⁵¹ taken together with the C atom to which they are attached form a cycloalkyl or heterocyclyl, wherein the cycloalkyl or heterocyclyl are optionally substituted by one or two groups selected from halogen, hydroxy, oxo, C₁₋₇-alkyl, C₁₋₇-haloalkyl, C₁₋₇-alkylcarbonyl, C₁₋₇-alkyloxycarbonyl, C₁₋₇-alkoxy, C₁₋₇-alkoxyalkyl, (R^{a})₂(R^{b})N— and C₁₋₇-alkyl-S(O)₀₋₂—, wherein each R^{a} is independently, at each occurrence, hydrogen or C₁₋₇-alkyl and R^{b} is an electron pair, hydrogen or C₁₋₇-alkyl;
Y is CR⁶⁰R⁶¹, O, NR⁶² or a direct bond, provided that when Y is O, Z is not O or S(O)₀₋₂, wherein:
R⁶⁰ and R⁶¹ are the same or different and independently selected from H and C₁₋₇-alkyl; and
R⁶² is selected from H, C₁₋₇-alkyl, C₁₋₇-alkylcarbonyl, aminocarbonyl, C₁₋₇-alkylaminocarbonyl, C₁₋₇-alkylsulfone, cycloalkylalkyl, cycloalkyl, aralkyl and aryl, wherein the C₁₋₇-alkyl, C₁₋₇-alkylcarbonyl, aminocarbonyl, C₁₋₇-alkylaminocarbonyl, C₁₋₇-alkylsulfone, cycloalkylalkyl, cycloalkyl, aralkyl and aryl are optionally substituted with one or more substitutents selected from halogen, hydroxy, oxo, C₁₋₇-alkyl, C₁₋₇-haloalkyl, C₁₋₇-alkylcarbonyl, C₁₋₇-alkyloxycarbonyl, C₁₋₇-alkoxy, C₁₋₇-alkoxyalkyl, (R^{a})₂(R^{b})N— and C₁₋₇-alkyl-S(O)₀₋₂—, wherein each R^{a} is independently, at each occurrence, hydrogen or C₁₋₇-alkyl and R^{b} is an electron pair, hydrogen or C₁₋₇-alkyl;
Z is CR⁷⁰R⁷¹, O, S(O)₀₋₂ or a direct bond, wherein:
R⁷⁰ and R⁷¹ are the same or different and independently selected from H or C₁₋₇-alkyl;
or R⁷⁰ and R⁷¹ taken together to form oxo (═O);
or Z and R⁸ or R¹² taken together form a cycloalkyl or heterocyclyl, wherein the cycloalkyl or heterocyclyl are optionally substituted by one or two groups selected from halogen, hydroxy, oxo, C₁₋₇-alkyl, C₁₋₇-haloalkyl, C₁₋₇-alkylcarbonyl, C₁₋₇-alkyloxycarbonyl, C₁₋₇-alkoxyalkyl, (R^{a})₂(R^{b})N— and C₁₋₇-alkyl-S(O)₀₋₂—, wherein each R^{a} is independently, at each occurrence, hydrogen or C₁₋₇-alkyl and R^{b} is an electron pair, hydrogen or C₁₋₇-alkyl;
R⁸, R⁹, R¹¹ and R¹² are the same or different and independently selected from: Q, hydrogen, C₁₋₇-alkyl, C₂₋₇-alkenyl, C₂₋₇-alkynyl, halogen, halogen-C₁₋₇-alkyl, C₁₋₇-alkoxy, halogen-C₁₋₇-alkoxy, hydroxy, hydroxy-C₁₋₇-alkoxy, hydroxy-C₁₋₇-alkyl, hydroxy-C₃₋₇-alkenyl, hydroxy-C₃₋₇-alkynyl, cyano, carboxyl, C₁₋₇-alkoxycarbonyl, amino carbonyl, carboxyl-C₁₋₇-alkyl, carboxyl-C₂₋₇-alkenyl, carboxyl-C₂₋₇-alkynyl, C₁₋₇-alkoxycarbonyl-C₁₋₇-alkyl, C₁₋₇-alkoxycarbonyl-C₂₋₇-alkenyl, C₁₋₇-alkoxycarbonyl-C₂₋₇-alkynyl, carboxyl-C₁₋₇-alkoxy, C₁₋₇-alkoxycarbonyl-C₁₋₇-alkoxy, carboxyl-C₁₋₇-alkyl-aminocarbonyl, carboxyl-C₁₋₇-alkyl-(C₁₋₇-alkylamino)-carbonyl, C₁₋₇-alkoxycarbonyl-C₁₋₇-alkyl-aminocarbonyl, C₁₋₇-alkoxycarbonyl-C₁₋₇-alkyl-(C₁₋₇-alkylamino)-carbonyl, carboxyl-C₁₋₇-alkyl-aminocarbonyl-C₁₋₇-alkyl, carboxyl-C₁₋₇-alkyl-(C₁₋₇-alkylamino)-carbonyl-C₁₋₇-alkyl, C₁₋₇-alkoxycarbonyl-C₁₋₇-alkyl-aminocarbonyl-C₁₋₇-alkyl, C₁₋₇-alkoxycarbonyl-C₁₋₇-alkyl-(C₁₋₇-alkylamino)-carbonyl-C₁₋₇-alkyl, hydroxy-C₁₋₇-alkyl-aminocarbonyl, di-(hydroxy-C₁₋₇-alkyl)aminocarbonyl, aminocarbonyl-C₁₋₇-alkyl-amino carbonyl, hydroxysulfonyl-C₁₋₇-alkyl-aminocarbonyl, hydroxysulfonyl-C₁₋₇-alkyl-(C₁₋₇-alkyl-amino)-carbonyl, di-(C₁₋₇-alkoxycarbonyl-C₁₋₇-alkyl)-methylaminocarbonyl, phenyl, wherein phenyl is unsubstituted or substituted by one to three groups selected from halogen, C₁₋₇-alkoxy, carboxyl or C₁₋₇-alkoxycarbonyl, phenyl-carbonyl, wherein phenyl is unsubstituted or substituted by one to three groups selected from halogen, C₁₋₇-alkoxy, carboxyl or C₁₋₇-alkoxycarbonyl, phenyl-aminocarbonyl, wherein phenyl is unsubstituted or substituted by one to three groups selected from halogen, C₁₋₇-alkoxy, carboxyl or C₁₋₇-alkoxycarbonyl, phenyl-C₁₋₇-alkyl, wherein phenyl is unsubstituted or substituted by one to three groups selected from halogen C₁₋₇-alkoxy, carboxyl or C₁₋₇-alkoxycarbonyl, phenyl-C₂₋₇-alkynyl, wherein phenyl is unsubstituted or substituted by one to three groups selected from halogen, C₁₋₇-alkoxy, carboxyl or C₁₋₇-alkoxycarbonyl, heteroaryl, wherein heteroaryl is unsubstituted or substituted by one to three groups selected from halogen C₁₋₇-alkoxy, carboxyl or C₁₋₇-alkoxycarbonyl, heteroaryl-carbonyl, wherein heteroaryl is unsubstituted or substituted by one to three groups selected from halogen, C₁₋₇-alkoxy, carboxyl or C₁₋₇-alkoxycarbonyl, heteroaryl-aminocarbonyl, wherein heteroaryl is unsubstituted or substituted by one to three groups selected from halogen, C₁₋₇-alkoxy, carboxyl or C₁₋₇-alkoxycarbonyl, heteroaryl-C₁₋₇-alkyl, wherein heteroaryl is unsubstituted or substituted by one to three groups selected from halogen, C₁₋₇-alkyl, C₁₋₇-alkoxy, carboxyl or C₁₋₇-alkoxycarbonyl, heteroaryl-C₁₋₇-alkyl-aminocarbonyl, wherein heteroaryl is unsubstituted or substituted by one to three groups selected from halogen, C₁₋₇-alkoxy, carboxyl or C₁₋₇-alkoxycarbonyl, heteroaryl-carbonyl-C₁₋₇-alkyl, wherein heteroaryl is unsubstituted or substituted by one to three groups selected from halogen, C₁₋₇-alkoxy, carboxyl or C₁₋₇-alkoxycarbonyl, and cycloalkyl, wherein cycloalkyl is unsubstituted or substituted by one to three groups selected from halogen, C₁₋₇-alkoxy, carboxyl or C₁₋₇-alkoxycarbonyl;
Q is: 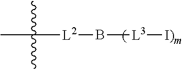
wherein:
L² and each L³ are either the same or different and independently absent, —O—, —NR⁸⁰—, —S—, —NR⁸⁰C(═O)—, —C(═O)NR⁸⁰—, —NR⁸⁰C(═O)NR⁸⁰—, —SO₂NR⁸⁰—, —NR⁸⁰SO₂—; —C₁₋₇alkylene-, —C₁₋₇alkylene-O—, —O—C₁₋₇alkylene-, —C₁₋₇ alkylene-NR⁸⁰—, —NR⁸⁰—C₁₋₇alkylene-, —C₁₋₇alkylene-S—, —S—C₁₋₇alkylene-, —C₁₋₇alkylene-NR⁸⁰C(═O)—, —C(═O)NR⁸⁰C₁₋₇alkylene-, —C₁₋₇alkylene-C(═O)NR⁸⁰—, —NR⁸⁰(═O)C₁₋₇alkylene-, —C₁₋₇alkylene-NR⁸⁰C(═O)NR⁸⁰—, —NR⁸⁰C(═O)NR⁸⁰C₁₋₇alkylene-, —C₁₋₇alkylene-SO₂NR⁸⁰—, —SO₂NR⁸⁰C₁₋₇alkylene-, —SO₂NR⁸⁰C(═O)—, —C(═O)NR80SO₂—, —NR⁸⁰SO₂NR⁸⁰C(═O)NR⁸⁰—, —NR⁸⁰C(═O)NR⁸⁰SO₂NR⁸⁰, —OC(═O)NR⁸⁰—, —NR⁸⁰C(═O)O—; —C₁₋₇alkylene-OC(═O)NR⁸⁰—, —NR⁸⁰C(═O)O—C₁₋₇alkylene-; —C₁₋₇alkylene-NR⁸⁰C(═O)O—, —OC(═O)NR⁸⁰—C₁₋₇alkylene-; —SO₂NR⁸⁰C₁₋₇alkylene- or —C₁₋₇alkylene-NR⁸⁰SO₂—;
B is optionally substituted C₁₋₇₀alkyl or C₁₋₇₀alkylene, wherein the C₁₋₇₀alkyl or C₁₋₇₀alkylene is optionally substituted with one or more functional groups selected from hydroxyl, oxo, carboxy, guanidino, amidino, —N(R⁸⁰)₂, —N(R⁸⁰)₃, phosphate, phosphonate, phospinate, sulfate, sulfonate and sulfinate, and wherein the C₁₋₇₀alkyl or C₁₋₇₀alkylene optionally, comprises one or more moieties selected from —NR⁸⁰—, —S—; —O—, —C₃₋₇cycloalkyl-, —C₃₋₇heterocyclyl-, —O₅₋₇heteroaryl-, —O₅₋₇aryl- and —SO₂—;
I is a compound of structure (XVIIa);
R⁸⁰ is independently, at each occurrence, hydrogen, C₁₋₇alkyl or —B-(L³-I)ₘ; and
m is an integer ranging from 0 to 10.

2-9. (canceled)

10. The compound of claim 1, wherein the compound has one of the following structures: 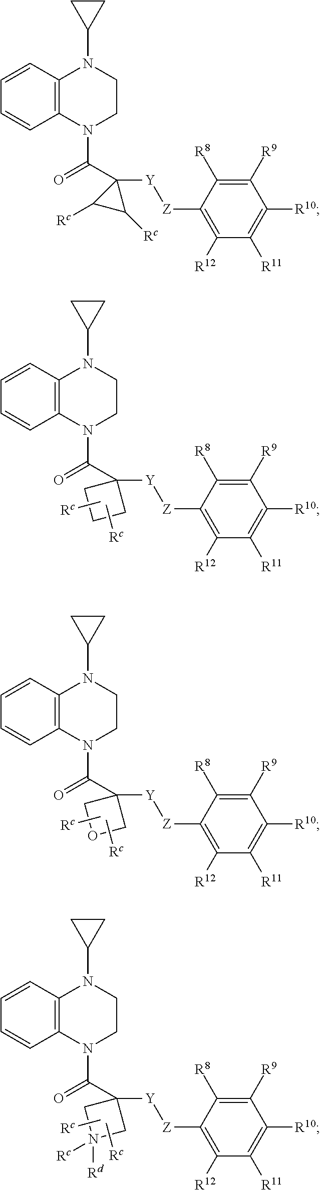 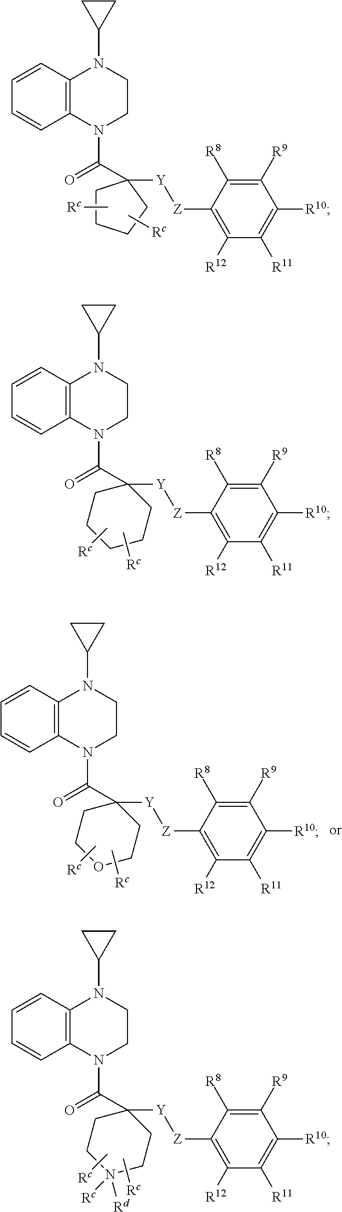
wherein:
R^{c} is independently, at each occurrence, hydrogen, halogen, hydroxy, oxo, C₁₋₇-alkyl, C₁₋₇-alkylcarbonyl, C₁₋₇-alkyloxycarbonyl, C₁₋₇-alkoxy, C₁₋₇-alkoxyalkyl or C₁₋₇-alkyl-S(O)₀₋₂—; and
R^{d} is independently, at each occurrence, an electron pair, hydrogen, C₁₋₇-alkyl, C₁₋₇-alkylcarbonyl, C₁₋₇-alkyloxycarbonyl, C₁₋₇-alkoxyalkyl or C₁₋₇-alkyl-S(O)₀₋₂—.

11. The compound of claim 10, wherein Y is O and Z is CR⁷⁰R⁷¹.

12. The compound of claim 10, wherein Y is NR⁶² and Z is CR⁷⁰R⁷¹.

13. The compound of claim 10, wherein Y is CR⁶⁰R⁶¹ and Z is O.

14-101. (canceled)

102. The compound of claim 1, wherein at least one of R⁸, R⁹, R¹⁰, R¹¹ or R¹² is halogen, C₁₋₇-alkyl, halogen-C₁₋₇-alkyl, C₁₋₇-alkoxy, halogen-C₁₋₇-alkoxy or cyano.

103. The compound of claim 102, wherein the halogen is chloro.

104-105. (canceled)

106. The compound of claim 1, wherein at least one of R⁸, R⁹, R¹⁰, R¹¹ or R¹² is Q.

107-109. (canceled)

110. The compound of claim 106, wherein L² is —O—, —C₁₋₇alkylene-; —C₁₋₇alkylene-NR⁸⁰—, —C₁₋₇alkylene-NR⁸⁰C(═O)—, —C₁₋₇alkylene-C(═O)NR⁸⁰— or —C₁₋₇alkylene-NR⁸⁰C(═O)NR⁸⁰—.

111. The compound of claim 106, wherein Q is -L²CR⁸¹R⁸²(CR⁸³R⁸⁴)ₘ₁G, wherein:
R⁸¹, R⁸², R⁸³ and R⁸⁴ are independently, at each occurrence, hydrogen or hydroxyl;
G is —CH₃, —CH₂OH, —CO₂H or -L³-I; and
m1 is an integer ranging from 1 to 21.

112. (canceled)

113. The compound of claim 111, wherein for each occurrence of R⁸³ and R⁸⁴, one of R⁸³ or R⁸⁴ is hydrogen and the other of R⁸³ or R⁸⁴ is hydroxyl.

114. The compound of claim 106, wherein Q has one of the following structures: 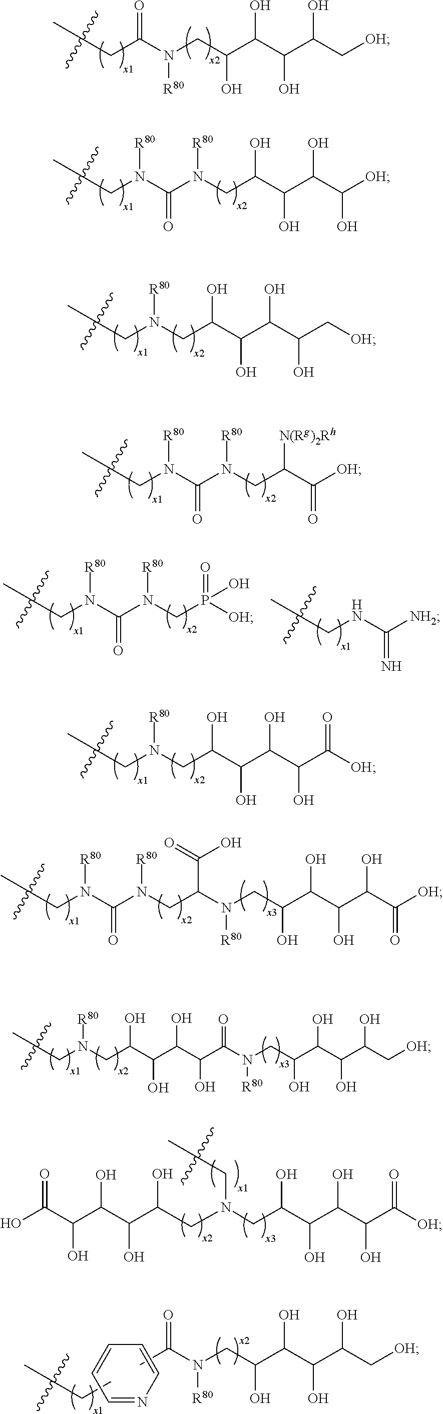 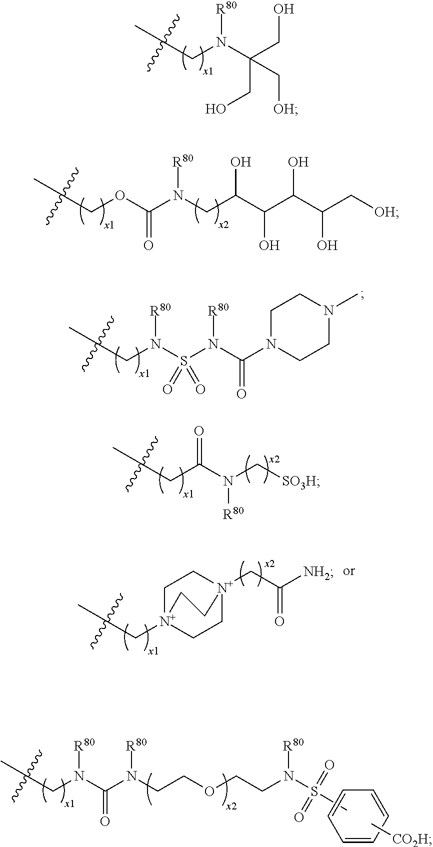
wherein:
R⁸⁰ is hydrogen or C₁₋₇alkyl;
R^{g} is independently, at each occurrence, hydrogen or C₁₋₇alkyl;
R^{h} is an electron pair, hydrogen or C₁₋₇alkyl; and
x1, x2 and x3 are each independently an integer ranging from 1 to 6.

115-116. (canceled)

117. The compound of claim 106, wherein Q is -L²[(CH₂)ₘ₂O]ₘ₃(CH₂)ₘ₂R⁸⁶, wherein m2 is 2 or 3, m3 is an integer ranging from 1 to 21 and R⁸⁶ is hydrogen, hydroxyl or L³-I.

118. (canceled)

119. The compound of claim 106, wherein Q has one of the following structures: 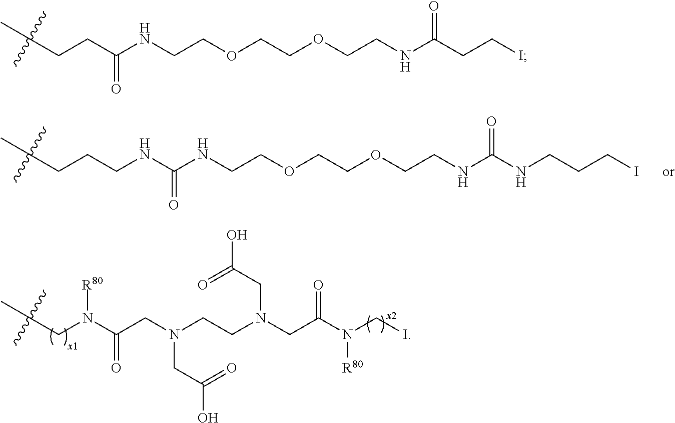
wherein I is a compound of structure (XVIIa).

120. The compound of claim 106, wherein B has the following structure:

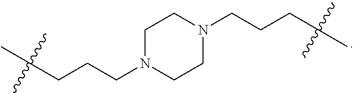 [SMILES: *CCCN1CCN(CCC*)CC1]

121-125. (canceled)

126. A compound of any one of Examples 1-291.

127. A pharmaceutical composition comprising a compound of claim 1 and a pharmaceutically acceptable carrier or adjuvant.

128. (canceled)

129. Use of a compound of claim 1 as a therapeutic active substance for the treatment of diseases which are associated with the modulation of TGR5 activity.

130. A method for the treatment of diseases which are associated with the modulation of TGR5 activity, wherein the diseases are selected from diabetes, Type II diabetes, gestational diabetes, impaired fasting glucose, impaired glucose tolerance, insulin resistance, hyperglycemia, obesity, metabolic syndrome, ischemia, myocardial infarction, retinopathy, vascular restenosis, hypercholesterolemia, hypertriglyceridemia, dyslipidemia or hyperlipidemia, lipid disorders such as low HDL cholesterol or high LDL cholesterol, high blood pressure, angina pectoris, coronary artery disease, atherosclerosis, cardiac hypertrophy, rheumatoid arthritis, asthma, chronic obstructive pulmonary disease (COPD), psoriasis, ulcerative colitis, Crohn's disease, disorders associated with parenteral nutrition especially during small bowel syndrome, irritable bowel syndrome (IBS), allergy diseases, fatty liver, non-alcoholic fatty liver disease (NAFLD), liver fibrosis, non-alcoholic steatohepatitis (NASH), primary sclerosing cholangitis (PSC), liver cirrhosis, primary biliary cirrhosis (PBC), kidney fibrosis, anorexia nervosa, bulimia nervosa and neurological disorders such as Alzheimer's disease, multiple sclerosis, schizophrenia and impaired cognition, the method comprising administering a therapeutically active amount of a compound of claim 1 or a pharmaceutical composition of claim 127 to a patient in need thereof.

131-190. (canceled)

191. The pharmaceutical composition of claim 127 further comprising one or more additional biologically active agents selected from dipeptidyl peptidase 4 (DPP-4) inhibitors, biguanidines, sulfonylureas, α-glucosidates inhibitors, thiazolidinediones, incretin mimetics, CB 1 antagonists, VPAC2 agonists, glucokinase activators, glucagon receptor antagonists, PEPCK inhibitors, SGLT1 inhibitors, SGLT2 inhibitors, IL-1 receptor antagonists, SIRT1 activators, SPPARMs and 11βHSD1 inhibitors.
